(19) United States
(12) Patent Application Publication
(10) Pub. No.: US 2023/0059141 A1
(43) Pub. Date: Feb. 23, 2023

(54) GENE EDITING SYSTEMS COMPRISING A NUCLEASE AND USES THEREOF

(71) Applicant: Arbor Biotechnologies, Inc., Cambridge, MA (US)

(72) Inventors: David A. Scott, San Francisco, CA (US); Quinton N. Wessells, Cambridge, MA (US); Lauren E. Alfonse, Boston, MA (US); Roy Ziblat, Newton, MA (US)

(21) Appl. No.: 17/815,846

(22) Filed: Jul. 28, 2022

Related U.S. Application Data
(60) Provisional application No. 63/227,625, filed on Jul. 30, 2021.

(51) Int. Cl. C12N 9/22; C12N 15/11; C12N 15/90

(52) U.S. Cl. CPC C12N 9/22; C12N 15/11; C12N 15/907; C12N 2310/20; C12N 2800/80

(57) ABSTRACT

The present invention relates to gene editing systems comprising nucleases or nucleic acids encoding the nucleases and RNA guides or nucleic acids encoding the RNA guides, processes for characterizing the gene editing systems, and methods or preparing and/or using the gene editing systems.

### CROSS-REFERENCE TO RELATED APPLICATION

The present application is entitled to priority under 35 U.S.C. § 119(e) to U.S. Provisional Patent Application No. 63/227,625, filed Jul. 30, 2021, which is hereby incorporated by reference in its entirety herein.

### REFERENCE TO AN ELECTRONIC SEQUENCE LISTING

The contents of the electronic sequence listing (sequencelisting.xml; Size: 565,519 bytes; and Date of Creation: Jul. 26, 2022) is herein incorporated by reference in its entirety.

### BACKGROUND

Clustered Regularly Interspaced Short Palindromic Repeats (CRISPR) and CRISPR-associated (Cas) genes, collectively known as CRISPR-Cas or CRISPR/Cas systems, are adaptive immune systems in archaea and bacteria that defend particular species against foreign genetic elements.

### SUMMARY OF THE INVENTION

It is against the above background that the present invention provides certain advantages and advancements over the prior art.

Although this invention disclosed herein is not limited to specific advantages or functionalities, the present invention provides, in one aspect, a gene editing system comprising:

(a) a nuclease or a nucleic acid encoding the nuclease, wherein the nuclease comprises an amino acid sequence with at least 80% identity to any one of SEQ ID NOs: 1-32; and

(b) an RNA guide or a nucleic acid encoding the RNA guide, wherein the RNA guide comprises a direct repeat sequence and a spacer sequence, wherein the nuclease binds to the RNA guide, and wherein the spacer sequence binds to a target nucleic acid.

In some embodiments, the nuclease comprises an amino acid sequence with at least 80% or at least 95% identity to SEQ ID NO: 26 or 27. In other embodiments, the nuclease comprises the amino acid sequence of SEQ ID NO: 26 or 27.

In another aspect, the present invention provides a cell comprising a gene editing system comprising:

(a) a nuclease or a nucleic acid encoding the nuclease, wherein the nuclease comprises an amino acid sequence with at least 80% identity to any one of SEQ ID NOs: 1-32; and

(b) an RNA guide or a nucleic acid encoding the RNA guide,

wherein the RNA guide comprises a direct repeat sequence and a spacer sequence, wherein the nuclease binds to the RNA guide, and wherein the spacer sequence binds to a target nucleic acid.

In some embodiments, the nuclease comprises an amino acid sequence with at least 80% or at least 95% identity to SEQ ID NO: 26 or 27. In other embodiments, the nuclease comprises the amino acid sequence of SEQ ID NO: 26 or 27.

In some aspects, the present invention provides a method of binding a gene editing system to a target nucleic acid in a cell, the method comprising:

(a) providing the gene editing system, wherein the gene editing system comprises a nuclease or a nucleic acid encoding the nuclease, wherein the nuclease comprises an amino acid sequence with at least 80% identity to any one of SEQ ID NOs: 1-32; and an RNA guide or a nucleic acid encoding the RNA guide, wherein the RNA guide comprises a direct repeat sequence and a spacer sequence, wherein the nuclease binds to the RNA guide, and wherein the spacer sequence binds to a target nucleic acid; and

(b) delivering the gene editing system to the cell,

wherein the cell comprises the target nucleic acid, wherein the nuclease binds to the RNA guide, and wherein the spacer sequence binds to the target nucleic acid.

In some embodiments, the nuclease comprises an amino acid sequence with at least 80% or at least 95% identity to SEQ ID NO: 26 or 27. In other embodiments, the nuclease comprises the amino acid sequence of SEQ ID NO: 26 or 27.

In other aspects, the present invention provides a method of introducing an indel into a target nucleic acid in a cell, the method comprising:

(a) providing a gene editing system comprising a nuclease or a nucleic acid encoding the nuclease, wherein the nuclease comprises an amino acid sequence with at least 80% identity to any one of SEQ ID NOs: 1-32; and an RNA guide or a nucleic acid encoding the RNA guide, wherein the RNA guide comprises a direct repeat sequence and a spacer sequence, wherein the nuclease binds to the RNA guide, and wherein the spacer sequence binds to a target nucleic acid; and

(b) delivering the gene editing system to the cell,

wherein recognition of the target nucleic acid by the gene editing system results in a modification of the target nucleic acid.

In some embodiments, the nuclease comprises an amino acid sequence with at least 80% or at least 95% identity to SEQ ID NO: 26 or 27. In other embodiments, the nuclease comprises the amino acid sequence of SEQ ID NO: 26 or 27.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

FIG. 1 is a plot showing the percentage of NGS reads comprising indels in HEK293 cells across AAVS1, EMX1, and VEGFA targets following transfection of the nuclease polypeptide of SEQ ID NO: 26.

FIG. 2 is a plot showing the percentage of NGS reads comprising indels in HEK293 cells across AAVS1, EMX1, and VEGFA targets following transfection of the nuclease polypeptide of SEQ ID NO: 27.

### DETAILED DESCRIPTION

In one aspect, the present invention provides novel nucleases and methods of use thereof. In some aspects, a gene editing system, kit, or cell comprising a nuclease of the present invention having one or more characteristics is described herein. In some aspects, a method of preparing a nuclease of the present invention is described. In some aspects, a method of delivering a gene editing system comprising a nuclease of the present invention is described.

### Definitions

The present invention will be described with respect to particular embodiments, but the invention is not limited thereto but only by the claims. Terms as set forth hereinafter are generally to be understood in their common sense unless indicated otherwise.

Unless otherwise defined, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

Generally, nomenclature used in connection with cell and tissue culture, molecular biology, immunology, microbiology, genetics, and protein and nucleic acid chemistry and hybridization described herein is well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

That the disclosure may be more readily understood, select terms are defined below.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, the term “activity” refers to a biological activity. In some embodiments, activity includes enzymatic activity, e.g., catalytic ability of a nuclease.

As used herein, the term “catalytic residue” refers to an amino acid that activates catalysis. A catalytic residue is an amino acid that is involved (e.g., directly involved) in catalysis.

As used herein, the term “complex” refers to a grouping of two or more molecules. In some embodiments, the complex comprises a polypeptide and a nucleic acid molecule interacting with (e.g., binding to, coming into contact with, adhering to) one another. For example, the term “complex” can refer to a grouping of an RNA guide and a nuclease polypeptide. Alternatively, the term “complex” can refer to a grouping of an RNA guide, a nuclease polypeptide, and the complementary region of a target sequence.

As used herein, the terms “domain” and “protein domain” refer to a distinct functional and/or structural unit of a polypeptide. In some embodiments, a domain may comprise a conserved amino acid sequence. As used herein, the term “RuvC domain” refers to a conserved domain or motif of amino acids having nuclease (e.g., endonuclease) activity. As used herein, a protein having a split RuvC domain refers to a protein having two or more RuvC motifs, at sequentially disparate sites within a sequence, that interact in a tertiary structure to form a RuvC domain.

As used herein, the term “nuclease” refers to an enzyme capable of cleaving a phosphodiester bond. A nuclease hydrolyzes phosphodiester bonds in a nucleic acid backbone. As used herein, the term “endonuclease” refers to an enzyme capable of cleaving a phosphodiester bond between nucleotides.

As used herein, the terms “parent,” “parent polypeptide,” and “parent sequence” refer to an original polypeptide (e.g., reference or starting polypeptide) to which an alteration is made to produce a variant polypeptide of the present invention.

The “percent identity” (a.k.a., sequence identity) of two nucleic acids or of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST nucleotide searches can be performed with the NBLAST program, score=100, wordlength-12 to obtain nucleotide sequences homologous to the nucleic acid molecules of the present disclosure. BLAST protein searches can be performed with the XBLAST program, score=50, word length=3 to obtain amino acid sequences homologous to the protein molecules of the present disclosure. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

As used herein, the term “protospacer adjacent motif” or “PAM” refers to a DNA sequence adjacent to a target sequence to which a complex comprising an RNA guide and a nuclease polypeptide binds. In a double-stranded DNA molecule, the strand containing the PAM motif is called the “PAM-strand” and the complementary strand is called the “non-PAM strand.” The RNA guide binds to a site in the non-PAM strand that is complementary to a target sequence disclosed herein. In some embodiments, the PAM strand is a coding (e.g., sense) strand. In other embodiments, the PAM strand is a non-coding (e.g., antisense strand). Since an RNA guide binds the non-PAM strand via base-pairing, the non-PAM strand is also known as the target strand, while the PAM strand is also known as the non-target strand.

As used herein, the term “adjacent to” refers to a nucleotide or amino acid sequence in close proximity to another nucleotide or amino acid sequence. In some embodiments, a nucleotide sequence is adjacent to another nucleotide sequence if no nucleotides separate the two sequences (i.e., immediately adjacent). In some embodiments, a nucleotide sequence is adjacent to another nucleotide sequence if a small number of nucleotides separate the two sequences (e.g., about 1, 2, 3, 4, 5, 6, 7, 8, 9, 10, 11, 12, 13, 14, 15, 16, 17, 18, 19, or 20 nucleotides).

As used herein, the terms “reference composition,” “reference sequence,” “reference gene editing system,” and “reference” refer to a control, such as a negative control or a parent (e.g., a parent sequence, a parent protein, a wild-type protein, or a complex comprising a parent sequence).

As used herein, the term “RNA guide” or “RNA guide sequence” refers to any RNA molecule or a modified RNA molecule that facilitates the targeting of a nuclease polypeptide described herein to a target sequence. For example, an RNA guide can be a molecule that is designed to include sequences that are complementary to a specific nucleic acid sequence. An RNA guide may comprise a DNA targeting sequence (i.e., a spacer sequence) and a direct repeat (DR) sequence. In some instances, the RNA guide can be a modified RNA molecule comprising one or more deoxyribonucleotides, for example, in a DNA-binding sequence contained in the RNA guide, which binds a sequence complementary to the target sequence. In some examples, the DNA-binding sequence may contain a DNA sequence or a DNA/RNA hybrid sequence. The terms CRISPR RNA (crRNA), pre-crRNA and mature crRNA are also used herein to refer to an RNA guide. The RNA guide can further comprise a tracrRNA sequence. In some embodiments, the tracrRNA sequence is fused to the direct repeat sequence of the RNA guide. In some embodiments, the RNA guide is a single molecule RNA guide (e.g., an sgRNA).

As used herein, the term “complementary” refers to a first polynucleotide (e.g., a spacer sequence of an RNA guide) that has a certain level of complementarity to a second polynucleotide (e.g., the complementary sequence of a target sequence) such that the first and second polynucleotides can form a double-stranded complex via base-pairing to permit an effector polypeptide that is complexed with the first polynucleotide to act on (e.g., cleave) the second polynucleotide. In some embodiments, the first polynucleotide may be substantially complementary to the second polynucleotide, i.e., having at least about 80%, 81%, 82%, 83%, 84%, 85%, 86%, 87%, 88%, 89%, 90%, 91%, 92%, 93%, 94%, 95%, 96%, 97%, 98%, or 99% complementarity to the second polynucleotide. In some embodiments, the first polynucleotide is completely complementary to the second polynucleotide, i.e., having 100% complementarity to the second polynucleotide.

As used herein, the terms “single molecule guide RNA,” “single molecule RNA guide,” “single guide RNA,” “sgRNA,” and the like are used to refer to an RNA guide (comprising a direct repeat sequence and a spacer sequence) fused to a tracrRNA. The RNA guide and tracrRNA can be transcribed together as a single transcript (e.g., with intervening linker nucleotides). The RNA guide and tracrRNA can be covalently linked (e.g., linked by intervening nucleotides). In some embodiments, the 3′ end of the RNA guide is linked to the 5′ end of the tracrRNA. In some cases, the 5′ end of the RNA guide is linked to the 3′ end of the tracrRNA. In some cases, the “end of the RNA guide is linked to the 5′ end of the tracrRNA. In some cases, the 3′ end of the RNA guide is linked to the 3′ end of the tracrRNA.

As used herein, the term “spacer” or “spacer sequence” is a portion in an RNA guide that is the RNA equivalent of the target sequence (a DNA sequence). The spacer contains a sequence capable of binding to the non-PAM strand via base-pairing at the site complementary to the target sequence (in the PAM strand). Such a spacer is also known as specific to the target sequence. In some instances, the spacer may be at least 75% identical to the target sequence (e.g., at least 80%, at least 85%, at least 90%, at least 95%, at least 98%, or at least 99%), except for the RNA-DNA sequence difference. In some instances, the spacer may be 100% identical to the target sequence except for the RNA-DNA sequence difference.

As used herein, the term “substantially identical” refers to a sequence, polynucleotide, or polypeptide, that has a certain degree of identity to a reference sequence.

As used herein, the term “target nucleic acid” refers to a double-stranded nucleic acid comprising a target sequence. As used herein, the term “target sequence” refers to a DNA fragment adjacent to a PAM motif (on the PAM strand). The complementary region of the target sequence is on the non-PAM strand. A target sequence may be immediately adjacent to the PAM motif. Alternatively, the target sequence and the PAM may be separately by a small sequence segment (e.g., up to 5 nucleotides, for example, up to 4, 3, 2, or 1 nucleotide). A target sequence may be located at the 3′ end of the PAM motif or at the 5′ end of the PAM motif, depending upon the CRISPR nuclease that recognizes the PAM motif, which is known in the art. For example, a target sequence is located at the 3′ end of a PAM motif for a nuclease polypeptide as described herein.

As used herein, the terms “trans-activating crRNA” and “tracrRNA” refer to an RNA molecule involved in or required for the binding of an RNA guide to a target nucleic acid.

### I. Gene Editing Systems

In some aspects, the invention described herein comprises gene editing systems comprising a nuclease. In some embodiments, a gene editing system of the invention includes a nuclease, and the gene editing system has nuclease activity. In some aspects, the invention described herein comprises gene editing systems comprising a nuclease and an RNA guide. In some embodiments, a gene editing system of the invention includes a nuclease and an RNA guide sequence, and the RNA guide sequence directs the nuclease activity to a site-specific target. In some embodiments, a nuclease of the gene editing system of the present invention is a recombinant nuclease.

In some embodiments, the gene editing system described herein comprises an RNA-guided nuclease (e.g., a nuclease comprising multiple components). In some embodiments, a nuclease of the present invention comprises enzyme activity (e.g., a protein comprising a RuvC domain or a split RuvC domain). In some embodiments, the gene editing system comprises an RNA guide. In some embodiments, the gene editing system comprises a ribonucleoprotein (RNP) comprising a nuclease and an RNA guide.

In some embodiments, the gene editing system of the present invention includes a nuclease polypeptide described herein.

A. Nuclease Polypeptide

In one embodiment, the nuclease is an isolated or purified nuclease.

A nucleic acid sequence encoding a nuclease described herein may be substantially identical to a reference nucleic acid sequence if the nucleic acid encoding the nuclease comprises a sequence having least about 60%, least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to the reference nucleic acid sequence. The percent identity between two such nucleic acids can be determined manually by inspection of the two optimally aligned nucleic acid sequences or by using software programs or algorithms (e.g., BLAST, ALIGN, CLUSTAL) using standard parameters. One indication that two nucleic acid sequences are substantially identical is that the two nucleic acid molecules hybridize to each other under stringent conditions (e.g., within a range of medium to high stringency).

In some embodiments, a nuclease described herein is encoded by a nucleic acid sequence having at least about 60%, least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to a reference nucleic acid sequence.

A nuclease described herein may substantially identical to a reference polypeptide if the nuclease comprises an amino acid sequence having at least about 60%, least about 65%, least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to the amino acid sequence of the reference polypeptide. The percent identity between two such polypeptides can be determined manually by inspection of the two optimally aligned polypeptide sequences or by using software programs or algorithms (e.g., BLAST, ALIGN, CLUSTAL) using standard parameters. One indication that two polypeptides are substantially identical is that the first polypeptide is immunologically cross-reactive with the second polypeptide. Typically, polypeptides that differ by conservative amino acid substitutions are immunologically cross-reactive. Thus, a polypeptide is substantially identical to a second polypeptide, for example, where the two peptides differ only by a conservative amino acid substitution or one or more conservative amino acid substitutions.

In some embodiments, a nuclease of the present invention comprises a polypeptide sequence having 50, 60, 65, 70, 75, 80, 85, 90, 91, 92, 93, 94, 95, 96, 97, 98, 99 or 100% identity to any one of SEQ ID NOs: 1-32. In some embodiments, a nuclease of the present invention comprises a polypeptide sequence having greater than 50, 60, 65, 70, 75, 80, 85, 90, 91, 92, 93, 94, 95, 96, 97, 98, 99 or 100% identity to any one of SEQ ID NOs: 1-32. The amino acid sequences corresponding to SEQ ID NOs: 1-32 are shown in Table 1. The corresponding nucleic acid sequences are set forth in SEQ ID NOs: 33-64.

TABLE 1
Amino acid and nucleic acid sequences of nucleases of SEQ ID NOs: 1-32.

| SEQ | | SEQ | |
|---|---|---|---|
| ID | | ID | |
| NO | Amino Acid Sequence | NO | Nucleic Acid Sequence |
| 1 | MDEQKEAVIPKVARFR | 33 | ATGGATGAACAGAAGGAGGCCGTAATCCCCAAGGTCGCGCGGTTCC |
| | ILKPVPPTTWQELGEL | | GGATTCTCAAGCCGGTCCCGCCCACCACGTGGCAGGAGCTTGGTGA |
| | LRAVRYRVFRLANLAV | | GCTGCTGCGGGCGGTGCGTTACCGCGTATTTCGCTTGGCAAACCTG |
| | SEKYLQFHLWRTGRTE | | GCGGTGAGCGAGAAATACCTGCAGTTCCACCTGTGGCGAACGGGCC |
| | SLDVRTVNQLNRDLRK | | GGACGGAAAGTCTGGACGTGCGCACCGTGAACCAACTGAATCGCGA |
| | VLEEEKEDEEAEEKKN | | CCTTCGCAAGGTGCTGGAGGAGGAGAAGGAGGACGAGGAGGCGGAG |
| | QHDVDPARLSKTGALP | | GAGAAGAAGAACCAGCACGACGTCGACCCGGCGCGACTTTCCAAAA |
| | DTVVAALSQYRIRPLT | | CAGGCGCATTGCCCGACACGGTCGTCGCGGCATTGAGCCAGTACAG |
| | TGSKWSQVIRGQTALP | | GATCCGGCCCCTGACGACGGGATCCAAGTGGAGCCAGGTAATACGG |
| | TFRLGMPIPIRCDKPS | | GGACAAACGGCCTTGCCGACGTTCAGGCTGGGCATGCCGATCCCCA |
| | HRRLERMQDGSVQLDL | | TCCGATGTGACAAGCCGAGCCATCGGCGTCTGGAGCGGATGCAGGA |
| | MVTRKPYPRVMLGTRN | | TGGGTCGGTACAGTTGGACCTGATGGTCACACGTAAGCCCTATCCC |
| | VGGGQAAVLERLLDNP | | CGTGTGATGTTGGGCACACGCAACGTGGGGGGTGGGCAGGCGGCGG |
| | VQDPSGYRQRCFEVKQ | | TGTTGGAGCGTCTGCTCGATAATCCAGTGCAGGACCCCTCGGGGTA |
| | DVQTGKWWLYVTYCFP | | TCGTCAGCGGTGTTTTGAGGTCAAGCAGGATGTGCAAACGGGCAAG |
| | AEATARSRDTVVGVDV | | TGGTGGCTGTATGTGACATACTGCTTTCCCGCCGAGGCTACCGCAC |
| | GVSVPLYAALSHGHAR | | GGAGTCGCGACACCGTGGTGGGTGTGGACGTGGGCGTGTCGGTTCC |
| | LGHQHFGPLGKQIRNL | | TCTGTACGCCGCGCTAAGCCACGGACATGCGCGACTGGGGCATCAG |
| | QNQVVARRRSIQRAGR | | CACTTCGGGCCCTTGGGCAAACAAATCCGAAACCTGCAAAATCAGG |
| | RGVVDKTARAGHGVRR | | TCGTTGCGCGGCGACGGTCGATCCAGCGGGCCGGGCGGCGGGGGGT |
| | MLGGTEKLRGRIDRAY | | CGTGGACAAGACGGCCCGCGCCGGTCATGGGGTCAGACGCATGCTG |
| | TTLNHQLSAAVVRFAR | | GGCGGGACGGAGAAGCTGCGCGGTCGGATCGACCGGGCGTATACGA |
| | NHGAGAIQVEDLSGLQ | | CGTTGAACCATCAGCTCAGCGCCGCCGTGGTCCGCTTCGCGCGGAA |
| | DTLRGTFLGGRWRYDQ | | TCACGGTGCCGGCGCGATCCAGGTGGAGGATTTGTCGGGCCTGCAA |
| | LQRFIEYKAKEAGIEY | | GACACTCTGCGCGGAACGTTTCTCGGCGGACGCTGGCGCTATGATC |
| | HKVNAAFTSRRCSECG | | AGTTGCAGCGATTCATCGAATACAAGGCCAAGGAAGCCGGGATCGA |
| | VIHEGFTRTFRDQHGT | | GTATCACAAGGTGAATGCAGCGTTCACGTCGCGGCGGTGCAGTGAA |
| | QGRSARFECPACGYKA | | TGCGGGGTCATCCATGAGGGGTTTACCCGGACGTTTCGCGACCAAC |
| | DADYNAARNLSVVDIE | | ACGGGACGCAGGGCCGGTCGGCGCGGTTTGAATGTCCGGCGTGTGG |
| | ERIRVQCAEQGLKAPT | | CTACAAAGCAGACGCGGATTATAACGCGGCGCGAAATCTATCGGTG |
| | SAGEVDTEPEDL | | GTTGACATCGAGGAACGCATACGGGTACAATGCGCCGAACAGGGGT |
| | | | TGAAGGCCCCAACGTCGGCCGGTGAGGTGGACACCGAGCCGGAGGA |
| | | | TCTTTGA |
| 2 | MGESVKAIKLKILDMF | 34 | ATGGGCGAATCGGTAAAAGCAATAAAATTAAAGATACTGGATATGT |
| | LDPECTKQDDNWRKDL | | TTTTAGACCCCGAATGCACAAAGCAGGATGATAACTGGCGCAAAGA |
| | STMSRFCAEAGNMCLR | | TTTGTCTACTATGTCCAGATTCTGCGCTGAAGCGGGGAATATGTGT |
| | DLYNYFSMPKEDRISS | | CTGCGCGACCTGTATAATTACTTTTCAATGCCCAAGGAAGACCGTA |
| | KDLYNAMYHKTKLLHP | | TTTCCTCAAAAGACTTATATAACGCTATGTATCATAAAACTAAACT |
| | ELPGKVANQIVNHAKD | | TCTCCATCCTGAATTACCAGGTAAGGTAGCGAACCAAATAGTAAAC |
| | VWKRNAKLIYRNQISM | | CACGCTAAAGATGTTTGGAAACGCAACGCTAAACTCATTTATCGGA |
| | PTYKITTAPIRLQNNI | | ACCAAATCTCAATGCCTACATATAAGATAACAACAGCACCAATCCG |
| | YKLIKNKNKYIIDVQL | | GCTGCAAAATAACATTTATAAATTAATAAAAAATAAGAACAAATAC |
| | YSKEYSKDSGKGTHRY | | ATAATAGACGTACAGTTATACTCCAAGGAATACTCCAAGGATAGTG |
| | FLVAVRDSSTRMIFDR | | GTAAAGGCACTCATAGGTATTTTCTGGTAGCAGTTAGAGACTCATC |
| | IMSKDHIDSSKSYTQG | | AACCCGTATGATATTCGACCGTATTATGAGTAAGGACCATATTGAC |
| | QLQIKKDHQGKWYCII | | AGTAGTAAATCATACACGCAAGGACAACTCCAAATCAAGAAAGACC |
| | PYTFPTHETVLDPDKV | | ACCAGGGGAAATGGTATTGCATCATACCCTATACATTCCCTACACA |
| | MGVDLGVAKAVYWAFN | | TGAGACAGTCCTCGACCCTGATAAAGTCATGGGAGTAGACCTTGGC |
| | SSYKRGCIDGGEIEHF | | GTTGCAAAAGCTGTTTACTGGGCGTTTAATAGTTCTTATAAAAGAG |
| | RKMIRARRVSIQNQIK | | GCTGTATCGACGGTGGGGAAATAGAACATTTCCGCAAAATGATACG |
| | HSGDARKGHGRKRALK | | AGCTCGCAGGGTGTCCATCCAAAATCAAATCAAACATTCAGGGGAC |
| | PIETLSEKEKNFRDTI | | GCCCGTAAAGGACATGGGCGCAAAAGGGCGTTAAAACCCATAGAAA |
| | NHRYANRIVEAAIKQG | | CATTGAGCGAGAAGGAAAAGAATTTTAGGGATACAATAAACCACCG |
| | CGTIQIENLEGIADTT | | CTATGCAAATCGAATTGTAGAAGCTGCTATTAAGCAAGGCTGTGGG |
| | GSKFLKNWPYYDLQTK | | ACAATCCAAATCGAAAACCTTGAAGGTATAGCTGACACAACAGGCA |
| | IVNKAKEHGITVVAIN | | GTAAATTTCTCAAGAACTGGCCTTATTACGACCTGCAGACAAAAAT |
| | PQYTSQRCSMCGYIEK | | TGTTAATAAAGCCAAGGAACATGGCATTACCGTTGTTGCAATAAAC |
| | TNRSSQAVFECKQCGY | | CCCCAATATACATCCCAAAGGTGTTCGATGTGCGGGTATATTGAAA |
| | GSRTICINCRHVQVSG | | AAACCAACCGTTCATCACAGGCAGTATTTGAATGTAAACAATGCGG |
| | DVCEECGGIVKKENVN | | TTACGGCAGTAGGACTATATGTATTAACTGCAGGCACGTCCAAGTA |
| | ADYNAAKNISTPYIDQ | | TCCGGGGATGTTTGTGAGGAATGTGGCGGCATAGTAAAAAAAGAAA |
| | IIMEKCLELGIPYRSI | | ACGTAAACGCAGACTACAATGCGGCAAAAAACATATCCACACCGTA |
| | TCKECGHIQASGNTCE | | CATCGACCAGATAATAATGGAGAAGTGTTTAGAACTAGGTATTCCT |
| | VCGSTNILKPKKIRKA | | TACCGCAGTATAACCTGTAAAGAATGTGGTCACATACAGGCTTCAG |
| | K | | GAAATACCTGCGAGGTTTGCGGAAGTACTAATATTTTGAAACCAAA |
| | | | GAAAATTAGAAAAGCAAAATAA |
| 3 | MNRIYQGRITGILDSK | 35 | ATGAACCGTATTTACCAAGGTCGGATTACCGGCATTCTTGATTCCA |
| | EDERGHPPPPDPKHNP | | AGGAGGACGAGCGGGGCCACCCTCCCCCACCCGATCCGAAACACAA |
| | FWRHHEVYQAAVNYYY | | TCCATTCTGGCGGCATCACGAGGTGTACCAGGCGGCGGTGAACTAC |
| | VAFAALGRAASDGMLR | | TACTATGTGGCCTTTGCGGCCTTGGGGCGCGCCGCATCGGACGGGA |
| | DLVQRVGESWETDPVH | | TGTTGCGCGACTTGGTGCAACGGGTCGGCGAATCCTGGGAGACCGA |
| | FRPEYGLRASLRQFLP | | TCCGGTTCACTTCCGACCCGAGTACGGGCTCAGAGCTTCGCTGAGA |
| | LSDTSTLEEAFERILE | | CAGTTTCTGCCTCTTTCCGACACCAGCACGCTCGAAGAGGCCTTCG |
| | KDHASPDTLLKAAMAV | | AACGAATTCTCGAAAAAGACCACGCTTCTCCCGACACCCTTCTCAA |
| | AKDLGGESSIQMKGRE | | AGCGGCCATGGCGGTGGCGAAAGATCTGGGGGGAGAGTCCAGCATC |
| | YLPRLCVPGYKGKFPR | | CAAATGAAAGGCCGGGAATATTTGCCCCGTCTCTGTGTGCCCGGAT |
| | EKNSLEKERMKVLLPV | | ACAAGGGGAAGTTTCCCCGCGAAAAAAACAGTCTGGAAAAGGAACG |
| | WLHAEDPASQPTLKKI | | AATGAAGGTGTTGCTGCCAGTGTGGCTCCACGCCGAGGACCCCGCC |
| | RFHHFANPTGTRLEVA | | TCGCAACCCACCTTGAAGAAAATCCGCTTCCACCACTTCGCCAACC |
| | ESKDLLGVAIDTLLRE | | CCACGGGAACCAGACTGGAAGTCGCTGAATCCAAAGACCTCCTCGG |
| | NKITAEEAGTLHDEID | | CGTCGCGATCGATACCCTCTTGCGGGAAAACAAAATCACCGCGGAG |
| | ALPDTFTLPAYAGGSV | | GAAGCGGGCACGCTCCATGACGAAATCGACGCCCTGCCCGACACCT |
| | NKDALKLRFHAYLITE | | TCACCCTGCCCGCCTACGCCGGGGGAAGCGTCAACAAAGACGCGCT |
| | HFWKNGRGLELLRSTY | | GAAACTCCGGTTCCACGCTTATTTGATTACCGAACATTTTTGGAAA |
| | PPPKGKPKKVGLSEEE | | AACGGCCGAGGCCTCGAATTGCTCCGCTCCACCTATCCTCCACCCA |
| | EALLVDGDDPVKVARG | | AAGGAAAACCAAAAAAGGTCGGACTTTCCGAGGAGGAGGAAGCCCT |
| | DRGYVFRAFTSLPAWG | | GCTGGTGGACGGAGACGATCCGGTCAAGGTGGCCCGGGGAGATCGA |
| | GRSAKDIAWKEFDIAA | | GGGTACGTCTTCCGCGCGTTCACCAGCCTTCCCGCCTGGGGCGGAC |
| | FKEALTTYNQFKDKTE | | GGAGCGCAAAGGACATCGCCTGGAAGGAATTCGACATCGCCGCCTT |
| | ERAARLRDVQARLARM | | CAAAGAGGCGCTGACCACCTACAATCAATTCAAAGATAAAACCGAG |
| | NGNEPEIPLAKEDKDD | | GAACGCGCGGCGCGGTTGCGGGACGTTCAGGCCCGACTCGCCCGAA |
| | IPRLENDPRVALLGEL | | TGAACGGCAACGAACCGGAAATTCCGCTGGCGAAGGAGGACAAGGA |
| | LHDSGVVAEGQTVDRG | | CGATATTCCCCGACTGGAGAACGATCCACGGGTGGCGCTTCTGGGA |
| | IYHRSLRHYRDLRSEW | | GAGCTTTTGCATGACTCGGGCGTCGTCGCGGAAGGCCAAACCGTCG |
| | NRVLSRASDGDSDSDI | | ACCGGGGAATTTACCACCGGTCCTTGCGGCATTACCGGGACCTTCG |
| | SKRLISVVNEMQAKYA | | ATCCGAGTGGAACCGTGTTCTCAGCCGGGCCTCTGACGGAGATTCG |
| | HTFGDVNLFRGLCSDP | | GACTCGGACATCTCAAAACGGCTGATTTCCGTCGTCAACGAAATGC |
| | KYWPVWKHPDAETKKR | | AGGCAAAGTACGCCCACACCTTCGGGGACGTGAACTTGTTCCGGGG |
| | IEKEGWAENVVEAYRD | | CTTGTGTTCCGATCCCAAATACTGGCCGGTGTGGAAACATCCCGAT |
| | FLELQAEEERYSEAIH | | GCCGAAACGAAAAAACGAATTGAAAAGGAGGGCTGGGCGGAAAACG |
| | FRPAHPEESSRYFRFS | | TCGTCGAGGCCTACCGAGACTTTCTCGAACTTCAGGCGGAGGAGGA |
| | DVVTKNQTLHEQPGER | | GCGCTATTCCGAAGCCATCCATTTCCGCCCGGCACACCCCGAGGAA |
| | LKLPIVERNDEGVYTK | | TCTTCGCGGTACTTCCGGTTTTCCGACGTCGTCACCAAGAATCAAA |
| | SSIRVRYSAPRLYRDA | | CCTTGCATGAACAACCCGGAGAGCGGCTCAAGCTTCCGATCGTCGA |
| | VCGNGNEKGHWLQPMV | | GAGGAACGACGAGGGCGTGTATACGAAATCGAGCATTCGCGTCCGC |
| | KALSLPEPPEADITNS | | TATTCGGCCCCCCGCCTTTATCGTGACGCCGTGTGTGGGAATGGAA |
| | AIVLNIKTDGENSSDK | | ACGAAAAGGGGCATTGGTTGCAGCCCATGGTCAAAGCGCTCTCACT |
| | RVRAYVDFPVELDTSP | | GCCCGAGCCTCCCGAGGCGGACATCACGAACAGCGCGATCGTGCTC |
| | LKEHLGHTRKWAKQFN | | AACATCAAAACCGACGGGGAAAACAGCTCGGACAAGCGTGTGCGGG |
| | GQYDRGVPYPSAGGGL | | CCTACGTCGATTTCCCCGTGGAACTGGACACCTCCCCTCTCAAGGA |
| | YWPDMQGLPSDPWYEN | | ACACCTGGGCCATACCCGGAAATGGGCCAAGCAATTCAACGGCCAA |
| | PAIQASGFQVLGVDLG | | TACGACCGCGGCGTCCCCTATCCCTCCGCCGGCGGCGGCCTCTACT |
| | QRTAECHALIEIRCDG | | GGCCCGACATGCAGGGGCTTCCCTCCGACCCTTGGTACGAAAACCC |
| | KFPQKKDGTSRDILAT | | GGCGATACAAGCCTCCGGATTTCAAGTGCTCGGAGTGGACCTCGGC |
| | VGHDGTRTWEAVLLRA | | CAACGGACCGCGGAATGCCATGCCCTGATCGAAATTCGGTGCGACG |
| | GTGRLPGENAHQMEQG | | GGAAGTTCCCTCAAAAGAAGGATGGCACGTCCCGGGATATCCTCGC |
| | RRVREKGGRRGRKTSD | | CACGGTGGGCCATGATGGAACACGGACGTGGGAAGCCGTGCTGTTG |
| | EDYRIAVEILEKIGLR | | CGGGCCGGAACCGGACGGTTGCCCGGCGAGAACGCCCACCAAATGG |
| | EIAESHRPLNDFKYAP | | AACAGGGACGCCGCGTACGCGAAAAAGGGGGGCGACGGGGCCGCAA |
| | ELWDFVLRYLKWIRNR | | AACCTCCGACGAGGATTATCGGATCGCCGTGGAAATCCTGGAAAAA |
| | LGRLFFAAADLRDDEL | | ATCGGCCTGCGCGAAATCGCGGAATCCCACCGTCCCTTGAATGATT |
| | RDKVATRLEEYPIPGI | | TTAAATACGCCCCCGAACTCTGGGATTTCGTACTGCGGTACCTGAA |
| | ELPSPLKERHEEAAEL | | ATGGATTCGAAATCGACTGGGCCGCCTGTTTTTCGCCGCCGCCGAT |
| | CLARYTELLETFRSAL | | TTGCGGGACGACGAGCTCCGTGACAAGGTGGCGACCCGGTTGGAGG |
| | LLLTDQILPLRGRRWT | | AGTATCCCATTCCCGGAATCGAACTCCCTTCCCCGTTGAAGGAGCG |
| | WAPHPQFPESTHHILM | | GCACGAGGAAGCCGCGGAGCTGTGCCTTGCCCGATACACGGAACTG |
| | ETPEPSTKGRKIRGQR | | CTCGAGACCTTCCGCTCCGCGCTACTCCTGTTGACGGATCAGATCC |
| | GLSFNRIEQIQELRKQ | | TTCCTCTGCGTGGCCGCCGATGGACCTGGGCACCCCACCCCCAGTT |
| | FQALNRMEGWDIQAPH | | CCCGGAGAGCACGCACCACATCCTCATGGAAACGCCGGAGCCTTCC |
| | RPGRDEIRQSLPECCQ | | ACAAAAGGCCGAAAAATCCGAGGGCAACGCGGGCTCTCTTTCAACC |
| | PLLEKLDNLRTQRVNQ | | GAATTGAACAAATTCAGGAACTCCGAAAGCAGTTCCAGGCCCTGAA |
| | TAHLILQEALGVELAP | | TCGCATGGAGGGATGGGATATTCAGGCCCCCCATCGACCGGGACGG |
| | GEKNATGDEHGVYRVK | | GACGAGATCCGCCAAAGCCTTCCCGAGTGCTGCCAACCTCTGCTGG |
| | HGRRPVDIIVLEALGD | | AAAAACTGGACAACCTGCGCACCCAACGCGTCAACCAAACGGCCCA |
| | FKTSQKRGRGTNRRLA | | CCTGATTCTACAGGAGGCCTTGGGAGTCGAGCTGGCGCCCGGCGAA |
| | SWAHRGISAKLRELAE | | AAAAACGCGACGGGGGACGAACACGGCGTCTATCGAGTAAAACACG |
| | PFGIPVVETPPHNTST | | GAAGAAGGCCTGTGGACATCATCGTGTTGGAGGCGCTGGGGGATTT |
| | FHAFTGRPGYRAREMS | | CAAAACGTCTCAAAAACGCGGGCGGGGCACGAACCGCCGCCTCGCC |
| | GIEMERIQTKLEEKKT | | TCCTGGGCCCACCGTGGGATTTCGGCCAAATTGCGTGAACTGGCGG |
| | PKTFRETLIKAGIDAM | | AACCCTTCGGCATCCCCGTGGTGGAAACGCCTCCCCACAACACGTC |
| | RESGTPPEKMTLLVPQ | | GACGTTCCATGCCTTCACGGGCCGCCCCGGATACAGGGCTCGGGAA |
| | QMGELFLPLTGPDEPA | | ATGTCCGGAATCGAAATGGAGCGAATCCAGACAAAGCTCGAAGAAA |
| | LPPIQSDINAAINIAL | | AGAAAACGCCAAAGACCTTCCGGGAGACATTGATCAAAGCAGGAAT |
| | KMVSAPEAVHLRHTVR | | CGACGCCATGCGGGAAAGCGGGACCCCGCCCGAAAAAATGACCCTG |
| | FETPAGKPVTPGKGSK | | TTGGTTCCGCAACAAATGGGGGAGCTGTTTCTTCCTCTGACCGGGC |
| | LEKALAKRKNVTFEIG | | CCGACGAACCCGCGCTTCCTCCCATCCAATCGGACATCAACGCCGC |
| | DEFHAPQARIDRANLL | | GATCAATATCGCGTTGAAAATGGTCTCCGCTCCGGAGGCGGTCCAT |
| | GTPAGTVHTPIGSFDF | | TTGCGGCACACGGTGCGATTTGAAACCCCGGCGGGGAAACCGGTCA |
| | MSRDAMRNLLSHYKDD | | CTCCCGGCAAGGGAAGCAAACTCGAAAAAGCCCTGGCCAAACGGAA |
| | MFIQANKRALEKRGIS | | AAACGTGACCTTCGAAATCGGTGACGAATTCCATGCGCCGCAGGCG |
| | FPMTPSNHPFSAGDFM | | CGTATCGACCGGGCGAATCTGCTGGGGACACCGGCGGGCACGGTCC |
| | DEHFPM | | ACACGCCCATCGGCTCCTTCGACTTTATGTCCCGTGACGCCATGAG |
| | | | AAACCTCCTTTCCCACTACAAAGACGACATGTTTATTCAGGCCAAC |
| | | | AAACGGGCACTCGAAAAGCGGGGAATTTCGTTTCCCATGACACCCT |
| | | | CCAACCACCCCTTCTCGGCGGGTGATTTCATGGACGAGCATTTTCC |
| | | | CATGTAA |
| 4 | MATRIYQGRIVQARFE | 36 | ATGGCTACTCGTATTTATCAGGGACGTATTGTGCAGGCTCGTTTTG |
| | DENLPQDKESALAALE | | AAGATGAGAACCTGCCGCAGGATAAGGAATCTGCATTAGCCGCATT |
| | RTNRLFQDAVNYHLVA | | GGAAAGAACGAATCGTCTTTTTCAAGATGCTGTCAACTACCACTTG |
| | LAGMAEDGKETLGSRF | | GTCGCGTTGGCAGGGATGGCGGAAGATGGGAAAGAGACTCTGGGAA |
| | KKQVRAIWEDLPRSKV | | GTAGATTCAAGAAACAGGTGAGGGCAATATGGGAAGATTTGCCTAG |
| | GACTLQRSIARTLSLA | | AAGTAAGGTTGGTGCTTGTACGTTGCAGCGCTCCATAGCACGTACT |
| | DGVTFDDAVAHIYEGC | | TTGAGTCTGGCAGACGGAGTTACGTTTGATGATGCCGTGGCACATA |
| | DRLDVLPYVEHYVIEQ | | TTTATGAAGGGTGCGATAGGCTGGATGTTTTGCCGTATGTCGAGCA |
| | TQKGEGAIQQQGRELL | | TTACGTGATTGAACAAACACAGAAGGGGGAGGGAGCTATTCAACAA |
| | PKLCNSDFEGNFDYSI | | CAAGGACGAGAGCTTCTTCCTAAATTGTGTAATTCGGATTTTGAAG |
| | KERKANSGKQKLLREL | | GTAATTTTGATTATAGTATCAAAGAACGTAAGGCGAATTCTGGGAA |
| | NRDDVSDEELYALAQE | | ACAGAAGCTGCTGAGGGAGCTTAATCGCGATGATGTTTCTGATGAG |
| | MDLSWVVKTIPNENHD | | GAGTTATACGCTTTGGCGCAGGAAATGGACTTATCGTGGGTTGTGA |
| | EPLYYDPEEQEEKVIT | | AAACAATACCTAATGAAAATCATGATGAGCCCTTGTATTATGATCC |
| | SIHKLLSSLDNGNLKI | | TGAAGAACAGGAGGAAAAGGTAATAACTTCTATTCATAAATTATTG |
| | ATLPKYVQIDGFREQM | | TCATCTTTAGATAATGGGAACTTGAAAATAGCAACTCTCCCCAAGT |
| | RQDLLNRMPLKGLRLA | | ATGTGCAAATTGACGGTTTTCGTGAGCAAATGCGGCAGGATCTTCT |
| | KSSRGISVDVEQAGIY | | TAATCGAATGCCTTTAAAAGGTCTTCGCTTGGCTAAGAGTAGTCGG |
| | FMFYPGRISAAHLASK | | GGAATTTCCGTAGATGTGGAACAGGCGGGTATATACTTCATGTTTT |
| | LGKEKPKKTDEVREYD | | ATCCTGGCCGCATTTCTGCTGCGCATCTGGCGTCTAAACTTGGAAA |
| | CFSLENDPLILARGKR | | AGAGAAGCCTAAAAAAACGGATGAAGTAAGAGAGTATGATTGCTTC |
| | GYVYKGFSALPNWESS | | AGCCTGGAAAATGACCCGCTCATATTGGCTCGCGGAAAACGAGGCT |
| | DNRMYSKEWDILAFKE | | ACGTTTACAAAGGTTTTTCGGCATTGCCTAACTGGGAATCATCGGA |
| | ALKTLHGFELKTKERD | | TAACCGCATGTATTCCAAAGAATGGGATATTCTTGCGTTTAAGGAA |
| | AERAKYVAQLEYVERG | | GCTTTGAAAACGCTGCATGGTTTTGAGCTTAAAACGAAAGAACGTG |
| | KKTKDYVEPDEEKTVA | | ATGCAGAGCGGGCGAAGTATGTTGCGCAGTTAGAATATGTTGAGAG |
| | VLGGDYRFELLKKLVA | | AGGTAAAAAAACGAAGGATTATGTGGAACCTGATGAGGAAAAGACT |
| | EISPDPVTEYHISSRT | | GTGGCTGTATTAGGGGGTGATTATCGGTTTGAGCTTCTCAAAAAAC |
| | LNDYEEVRALWLKREL | | TTGTAGCGGAAATTTCGCCTGACCCTGTTACTGAATACCACATTTC |
| | TGDCSSHDLSKIVREY | | CTCACGAACACTGAATGATTATGAGGAAGTGAGGGCTCTGTGGTTG |
| | QAKSKRFGSQVLFAAL | | AAAAGGGAGCTCACGGGGGACTGCAGCAGCCACGATTTATCCAAGA |
| | CQDEYRPIWHDYHALK | | TTGTTCGTGAATATCAGGCTAAAAGCAAAAGATTTGGCTCCCAAGT |
| | DEKLPRSKNILRDFSN | | GCTGTTTGCAGCGCTGTGTCAGGATGAGTATCGGCCTATCTGGCAT |
| | WQYLCGQVEKYSRDVR | | GATTATCATGCATTGAAAGATGAGAAACTTCCTAGGTCCAAGAATA |
| | VTAADVVASPRQMIYS | | TACTGCGTGATTTTTCCAACTGGCAATACTTGTGCGGGCAGGTTGA |
| | DLSNFGNGKGCEYIPG | | GAAATACAGCCGTGATGTCAGGGTGACTGCTGCGGATGTGGTAGCA |
| | CEGGLRMQVVVRNAKG | | TCTCCTCGCCAGATGATTTATTCCGATCTTTCCAATTTTGGAAATG |
| | HWETDSIRVTFSAPRF | | GAAAGGGGTGTGAGTATATTCCCGGTTGTGAGGGTGGTTTGCGTAT |
| | LRDEMGQDAGKWMVPK | | GCAGGTGGTCGTTCGGAATGCTAAAGGACATTGGGAAACGGACTCG |
| | KGENTTLPWLQPMMKA | | ATTCGTGTTACTTTCTCAGCTCCAAGATTTTTGAGGGATGAGATGG |
| | LGDDIAPVRLERTPAI | | GGCAGGACGCTGGCAAATGGATGGTTCCTAAAAAGGGAGAAAATAC |
| | GLQVWGQGSEACYYLN | | GACCCTGCCGTGGTTGCAACCGATGATGAAGGCTTTGGGGGATGAT |
| | FPVSLDVSALQKSLGK | | ATTGCACCTGTTCGCCTGGAACGTACGCCTGCCATTGGTTTGCAGG |
| | AARWAGQFLGGRDEKL | | TTTGGGGGCAGGGCTCGGAGGCTTGTTATTACCTGAACTTCCCCGT |
| | HLHWPATYKGKNRPWW | | GTCACTGGATGTGTCTGCTCTTCAGAAGTCTCTTGGCAAGGCTGCT |
| | EQEKEFTVLGIDLGLR | | CGTTGGGCCGGTCAATTCCTCGGAGGCAGGGATGAGAAACTTCATT |
| | SSVAWSLLRVSTCSTS | | TGCACTGGCCTGCTACCTACAAAGGTAAGAATCGCCCTTGGTGGGA |
| | SNSRGDELIGRLIGDS | | ACAGGAAAAGGAGTTTACAGTGTTGGGCATAGATTTAGGTCTTCGT |
| | SNAKWYGYVIKQGLSR | | AGCTCGGTTGCCTGGTCACTGTTGCGTGTCTCAACCTGCTCTACTT |
| | LPGEQPRRVGDKKIPA | | CTTCTAACTCCAGAGGTGATGAGCTAATAGGTCGCTTGATAGGGGA |
| | VSLASPEDRRIAKLII | | TAGCTCAAACGCTAAATGGTATGGCTATGTGATTAAGCAAGGTTTA |
| | EAAGAQFDENESEDVL | | TCTCGCCTTCCGGGGGAGCAACCTCGTCGAGTTGGCGATAAAAAGA |
| | RLGNRTLKSFKSLTSR | | TTCCAGCCGTAAGTCTTGCCTCCCCTGAAGATAGAAGAATTGCCAA |
| | LKTYLSFLSGLKDPGR | | GTTGATTATTGAAGCTGCTGGTGCTCAGTTTGATGAGAATGAATCT |
| | KPAVLKRMADYFAYAE | | GAAGATGTGCTTCGTCTGGGCAATAGAACGCTCAAAAGCTTTAAGA |
| | IIPGVCVLLEAHQEDE | | GTTTGACAAGTCGACTGAAAACATACTTGTCTTTCTTGTCCGGGCT |
| | VYERVLDAAEELRSML | | TAAGGATCCTGGTAGAAAACCTGCTGTGTTAAAGCGCATGGCGGAT |
| | PKYAERVTSLILPRKH | | TATTTTGCGTACGCAGAGATAATACCCGGAGTCTGTGTTCTTCTGG |
| | GSWKWEPERRKGWQGS | | AAGCGCACCAGGAAGATGAGGTTTATGAGAGGGTTCTGGATGCAGC |
| | GVMRLTEEDGIPHRPV | | TGAAGAGCTGCGCAGTATGTTGCCTAAGTATGCAGAACGAGTAACA |
| | FHRGGLSVARLTQLET | | AGCCTTATTTTGCCTCGCAAACACGGGTCTTGGAAATGGGAGCCGG |
| | LRQLLQSMGKVLSFVP | | AGCGACGCAAGGGCTGGCAGGGCTCCGGCGTGATGCGCTTAACAGA |
| | GESVTFGRRLKDEKVI | | AGAGGACGGTATACCGCATCGACCAGTTTTCCATCGCGGTGGTTTG |
| | DPCPEILEKIENMREQ | | TCTGTTGCCCGTTTGACTCAGCTTGAAACGCTCAGGCAGCTGTTGC |
| | RVNLIAHDIVAQALGV | | AGTCCATGGGGAAAGTGTTATCATTTGTTCCTGGTGAATCTGTTAC |
| | RLKSSRPHKNSGGLDV | | ATTTGGACGCAGGTTAAAAGATGAAAAAGTCATTGATCCATGTCCT |
| | IHGEYERIHGREPVDF | | GAGATTTTGGAAAAAATCGAGAATATGCGCGAGCAACGCGTCAATT |
| | VVMENLSRYLTSLDRA | | TGATTGCTCACGACATCGTTGCTCAGGCATTGGGCGTTCGTTTGAA |
| | PLENSGLMRWAHRQIV | | ATCCTCTCGTCCTCACAAGAATTCAGGTGGGCTGGATGTTATTCAT |
| | AKVTQLLEEIFGIPVV | | GGCGAATATGAGCGTATTCACGGGCGAGAGCCTGTAGATTTTGTCG |
| | FTHAHYTSKFDSMTSE | | TAATGGAGAATCTCAGCCGTTATCTGACTTCTCTGGACCGGGCTCC |
| | PGFRPVMLKPEYLKWL | | GCTTGAGAACTCAGGGTTGATGCGTTGGGCTCACCGGCAGATTGTG |
| | QRNGKGNECKAAAVYQ | | GCAAAGGTGACGCAGCTCCTTGAGGAAATATTCGGCATTCCGGTTG |
| | AIWDEVVNASKTKKVT | | TCTTTACCCATGCTCATTACACTTCCAAGTTTGATTCAATGACCTC |
| | LVLPHLTKGGELANGG | | GGAACCAGGTTTCCGTCCCGTGATGTTGAAGCCGGAGTATCTGAAG |
| | ELFLSQKGGKFTLRNA | | TGGTTGCAACGAAATGGAAAAGGAAACGAATGTAAGGCGGCAGCTG |
| | DMNAATNVAWRGLAAP | | TATATCAAGCCATATGGGATGAAGTTGTGAATGCTAGCAAGACGAA |
| | ESLHLLHRVRMEMKKA | | AAAGGTGACTTTGGTATTGCCGCATTTGACAAAAGGAGGAGAGCTG |
| | GFVPVCDNAREKSLKT | | GCAAATGGAGGTGAGCTGTTCTTATCTCAGAAAGGGGGGAAGTTTA |
| | GWSLTKLKSIQPEGNK | | CCTTGCGCAATGCTGATATGAATGCGGCTACGAATGTCGCTTGGCG |
| | ISAFAVSSEWKDEYFA | | AGGATTGGCTGCCCCGGAATCGCTGCACTTGCTGCACCGGGTCCGT |
| | AYGNAETRAYLAYGKT | | ATGGAGATGAAGAAGGCTGGTTTTGTGCCGGTGTGCGACAATGCTC |
| | LWGIMKRKQWQMCHLF | | GCGAGAAAAGCTTGAAGACAGGGTGGAGTTTGACCAAGTTGAAATC |
| | NIQQLKNAGIDARLVE | | CATTCAGCCCGAAGGAAATAAGATATCCGCATTTGCTGTTTCTTCC |
| | QLLHTDENDTSDDIPT | | GAGTGGAAAGATGAGTACTTTGCTGCCTATGGTAATGCAGAGACGA |
| | | | GGGCTTATCTGGCTTACGGGAAAACGCTATGGGGGATAATGAAGAG |
| | | | AAAACAGTGGCAGATGTGCCATTTGTTCAATATTCAGCAGTTGAAA |
| | | | AATGCAGGCATAGACGCGCGCTTGGTGGAACAATTGCTGCATACTG |
| | | | ATGAAAACGACACATCGGATGATATTCCTACATGA |
| 5 | MFKTARFKVHNPSRHK | 37 | ATGTTCAAAACCGCGCGCTTCAAGGTTCACAATCCGTCACGGCACA |
| | STMLWYAMTRYHNTLK | | AGAGCACGATGCTCTGGTACGCGATGACTCGCTATCACAACACCTT |
| | DVLEKTLAIPNLLEQV | | GAAGGATGTCCTCGAAAAGACACTGGCCATCCCAAACCTGCTTGAA |
| | SELDKKEKLRPNKFKL | | CAAGTCTCCGAACTCGACAAAAAAGAAAAACTTCGGCCCAACAAAT |
| | SKLLYTIAPQNWELAP | | TCAAACTCAGCAAATTGCTTTACACGATCGCACCACAAAATTGGGA |
| | LRDYLIGDASAMLMSH | | ACTTGCCCCTCTGCGTGACTATCTCATCGGCGATGCCTCTGCGATG |
| | LNKTYKGANESNPPTV | | CTGATGAGCCATCTGAACAAGACCTACAAGGGCGCGAACGAGTCTA |
| | SSLNAMTDAEFHKAYS | | ACCCACCCACTGTCTCCAGCTTAAATGCAATGACTGACGCTGAGTT |
| | DFTDPEAKLAIKPQHQ | | TCACAAGGCCTACAGCGACTTTACGGACCCGGAGGCAAAGCTCGCC |
| | EKIDKAAERGETNVAN | | ATCAAACCGCAGCATCAGGAAAAGATCGACAAGGCTGCCGAGCGAG |
| | RLSKIYANWAASRAAG | | GCGAGACAAACGTCGCAAATCGTCTGAGCAAAATCTACGCGAATTG |
| | QVLRKLEGELPHPIEF | | GGCCGCCTCCCGAGCTGCGGGTCAGGTGCTGCGTAAACTAGAAGGT |
| | TRNEIGRGCLLAYCDG | | GAACTGCCTCACCCCATCGAATTCACACGTAACGAGATTGGGCGGG |
| | NYYLLVRLFAQDHRYC | | GTTGCTTGCTTGCCTATTGCGACGGTAACTACTATCTGCTCGTCCG |
| | EKRVLKEGFINCKTKE | | TCTATTCGCGCAAGATCATCGCTATTGTGAAAAGCGCGTTCTCAAA |
| | HIEGKKYPGMILPLEL | | GAAGGATTCATCAATTGCAAAACAAAGGAACACATTGAAGGCAAAA |
| | GREFHEREYLTHGSIQ | | AATATCCTGGAATGATTCTCCCTCTGGAACTGGGCCGCGAATTCCA |
| | SAKLIVKRREKPNSQP | | TGAGCGGGAATATCTCACGCATGGTTCCATCCAAAGCGCCAAGCTG |
| | KALDSKPAPFNAEDYD | | ATTGTGAAGCGGCGGGAAAAGCCAAACTCACAGCCGAAAGCCCTGG |
| | FYVHAAFEFQPTQIET | | ACTCAAAACCCGCCCCTTTTAACGCGGAAGACTACGATTTTTATGT |
| | ETFLGIDRGAAKIGAA | | TCACGCGGCCTTTGAGTTTCAGCCGACGCAGATCGAAACGGAGACA |
| | TLIDRQGKPLETDLDL | | TTCCTTGGCATCGACCGCGGCGCTGCCAAGATCGGCGCTGCCACCT |
| | SGAAFAAEMRRFEQQI | | TGATTGACCGACAGGGAAAGCCCCTTGAAACCGATCTCGATCTTAG |
| | KRIQKQGKQKSRKFSL | | CGGCGCAGCCTTTGCGGCGGAGATGCGGCGCTTTGAACAGCAGATC |
| | RGKRADIILGEYANRI | | AAGCGAATTCAGAAACAGGGAAAACAGAAGTCACGCAAGTTTTCAC |
| | VAIAKENRSQIVIEAI | | TACGCGGCAAACGAGCAGATATCATTCTGGGAGAGTACGCCAACCG |
| | KGVTMGRFLKQSQFTK | | GATTGTTGCTATTGCCAAAGAGAATCGCTCACAGATTGTCATCGAG |
| | LKQMLTYKAEREGLPA | | GCCATCAAGGGCGTGACAATGGGCAGATTTTTGAAACAAAGCCAAT |
| | PIEVPAAFTSQTCARC | | TCACCAAGCTGAAGCAGATGCTCACCTACAAGGCGGAGCGAGAGGG |
| | GHKDPANRPKKDAAGK | | ACTGCCAGCTCCTATCGAAGTTCCTGCGGCGTTCACTTCACAGACC |
| | AIQDVFLCTACGHHAN | | TGCGCCAGATGCGGCCATAAAGATCCGGCCAATCGTCCAAAGAAAG |
| | ADSNASLIIALRGLHQ | | ATGCGGCTGGAAAAGCGATTCAAGATGTGTTTTTATGCACGGCTTG |
| | IENGGKFKKFDLFQQW | | CGGTCATCATGCCAACGCCGATTCCAATGCGAGCCTGATTATTGCG |
| | LKEIIGRDGSFAPGQV | | CTGCGTGGGCTGCATCAGATTGAAAATGGTGGTAAATTCAAGAAGT |
| | SP | | TCGATCTCTTTCAACAATGGTTGAAGGAAATTATCGGCCGGGACGG |
| | | | CTCCTTTGCCCCAGGGCAGGTGAGCCCGTAG |
| 6 | MFKTARFKIHNPSRHK | 38 | ATGTTTAAGACGGCGCGGTTCAAGATTCACAACCCGTCACGCCACA |
| | QAVLRYALSHYHLTLK | | AGCAGGCTGTGCTCCGCTACGCCCTCTCCCACTACCACCTCACGCT |
| | NVLEAALADPELQARV | | GAAGAACGTGCTTGAGGCCGCCCTCGCCGATCCTGAGCTGCAGGCG |
| | TVVGKNGKPRTDKAAL | | CGGGTGACGGTGGTGGGCAAGAATGGTAAGCCGCGAACCGATAAGG |
| | SRFLYALAPKGWPLAP | | CTGCTCTGAGCCGATTTCTCTACGCTCTGGCCCCAAAAGGCTGGCC |
| | LRDYLIGDASAMLLSH | | GCTGGCGCCCTTGCGCGACTACCTGATAGGCGACGCAAGCGCTATG |
| | YEKDLKGKNESNPPTL | | CTGCTCAGCCACTACGAGAAGGACCTGAAGGGCAAGAACGAATCCA |
| | GGLEGLTEERRREAFH | | ATCCCCCGACGCTCGGCGGACTCGAAGGTTTGACGGAGGAGCGCCG |
| | DFVMTEEFPLRADRAA | | CCGGGAGGCGTTCCATGACTTCGTCATGACCGAAGAGTTTCCTCTT |
| | EIEKARSLGQVHLAKR | | CGTGCGGACCGCGCCGCCGAGATTGAAAAGGCGCGGTCTCTCGGCC |
| | LGNIYASRASANAMRD | | AAGTGCACCTTGCCAAGCGCCTGGGGAACATTTATGCGTCGCGGGC |
| | LLRSLDAPIPRPIEFT | | GAGCGCAAATGCCATGCGGGATCTGCTGCGGAGCCTGGACGCGCCG |
| | RCEFGRGFLLARKGDK | | ATTCCCCGGCCGATCGAGTTCACCAGATGCGAGTTTGGACGCGGTT |
| | FYLLLRLFSKGNSYWQ | | TCCTGCTGGCGCGAAAGGGTGACAAGTTCTATCTCCTGCTGCGGCT |
| | QVMLDEGFVNWKTKET | | CTTCTCGAAGGGAAACTCCTACTGGCAACAGGTCATGTTGGACGAG |
| | IGGRKYPGVVLPLEFG | | GGGTTCGTCAACTGGAAAACGAAGGAGACCATTGGCGGCCGGAAAT |
| | RDFHESEYLEHGSPQS | | ATCCGGGCGTGGTCCTTCCGTTGGAGTTCGGACGCGACTTCCACGA |
| | AKLILKRSDEGLEEFC | | AAGCGAGTACCTGGAGCATGGGTCGCCGCAGAGCGCGAAGCTGATT |
| | AHIAFEFTPEPVTPET | | TTGAAACGGAGCGATGAGGGTCTGGAGGAGTTCTGTGCTCACATCG |
| | FLGIDRGAAMIGAATV | | CTTTCGAATTCACCCCTGAACCCGTCACCCCCGAGACGTTTCTCGG |
| | IDQAGGLVTRRLDLEG | | TATCGATCGCGGAGCCGCTATGATCGGGGCGGCAACCGTGATCGAT |
| | TAFNRELKRFEFRIAK | | CAGGCTGGTGGTTTGGTTACGCGGCGCTTGGACCTGGAGGGAACGG |
| | AQRKAQRRPRLFRVRR | | CGTTCAACCGCGAATTGAAGAGGTTTGAATTTCGTATTGCAAAGGC |
| | RWAAIVIGEYANRVVA | | ACAACGGAAGGCACAGCGCAGGCCTCGACTGTTCCGGGTGCGGCGC |
| | EAVKHRSQIVLEKIDA | | CGTTGGGCCGCAATCGTCATCGGCGAATACGCTAACAGGGTTGTTG |
| | RSMARFLSHSQFRKLH | | CAGAGGCCGTCAAGCATCGCTCGCAGATTGTACTGGAGAAGATAGA |
| | DAITYKAERMGLPRPI | | CGCTCGCTCCATGGCCCGGTTCCTGAGCCACAGCCAGTTCCGGAAG |
| | EVPAAYTSQTCARCGH | | CTGCACGATGCCATTACTTATAAGGCCGAGCGCATGGGCTTACCAA |
| | RDAANRPKKDSEGRPL | | GGCCCATCGAAGTCCCGGCGGCATACACGTCGCAGACTTGTGCCCG |
| | QAVFRCVRCGCEANAD | | GTGCGGCCACCGGGATGCGGCGAACCGGCCGAAGAAAGACAGCGAG |
| | GNASEVIALRGLHQAL | | GGCCGCCCACTACAGGCCGTGTTCCGGTGTGTGCGGTGCGGGTGCG |
| | NGGKFQKFPAFQEWLV | | AAGCCAATGCCGACGGGAACGCCAGCGAGGTTATCGCTCTGAGGGG |
| | GIRRRVGEPALIGR | | TTTGCATCAGGCGCTCAATGGTGGTAAATTTCAGAAGTTCCCAGCC |
| | | | TTCCAGGAATGGTTGGTCGGGATCAGACGCCGGGTTGGTGAGCCAG |
| | | | CTTTAATCGGCCGGTGA |
| 7 | MFKTARFKVHNPSRHK | 39 | ATGTTCAAGACCGCCCGCTTCAAGGTTCACAACCCGTCACGGCATA |
| | STMLWYAMTRYHETLK | | AGAGCACGATGCTCTGGTACGCCATGACTCGCTATCACGAGACGCT |
| | AVLEETLAIPDLLEQV | | GAAGGCCGTTCTCGAAGAGACGCTGGCGATTCCCGATCTGCTGGAA |
| | SELDKKEKLRPNKFRL | | CAAGTCTCCGAGTTGGACAAGAAGGAAAAGCTCCGGCCAAACAAGT |
| | SKLLYTIVPKSWELAP | | TCCGGCTAAGCAAACTGCTCTACACAATTGTGCCCAAAAGCTGGGA |
| | LRDYLIGDTSAMLMSH | | ACTTGCTCCGCTGCGCGATTATCTCATAGGCGATACATCCGCGATG |
| | LSKAYKGENESNPPTV | | CTTATGAGCCACCTCAGCAAGGCGTACAAGGGAGAGAACGAGTCTA |
| | SSLAAMTDEEFRKAYS | | ATCCTCCGACGGTCTCAAGCTTGGCCGCAATGACCGACGAGGAGTT |
| | EFTDPEAQLAVKPQHQ | | TCGCAAGGCATACAGCGAGTTTACAGACCCGGAGGCGCAGCTTGCC |
| | EKIDKASERGETRVAK | | GTCAAGCCGCAGCATCAGGAAAAGATCGACAAGGCGAGCGAACGAG |
| | RLSKIYANWAVSRAAG | | GCGAGACCCGCGTCGCCAAACGCCTGAGCAAAATCTACGCCAATTG |
| | QVLRRLEGALPHPIEF | | GGCCGTCTCTCGCGCCGCAGGGCAGGTGCTACGCAGGCTGGAAGGC |
| | TRNEFGRGCLLAFCDG | | GCGCTGCCCCATCCCATTGAGTTCACCCGCAACGAGTTCGGGCGGG |
| | NYYLLVRLFAQGHRYC | | GCTGCCTACTTGCTTTTTGCGACGGAAACTACTATCTGCTCGTCCG |
| | EKRVLKDGFIDCKTKE | | GCTCTTCGCCCAAGGCCACCGTTATTGCGAAAAGCGGGTACTGAAA |
| | RLDGKRYPGLILPLEL | | GACGGCTTCATCGACTGCAAGACAAAGGAGCGGCTCGACGGCAAGA |
| | GREFHEREYLTYGAIQ | | GATATCCCGGCCTGATTCTGCCGCTTGAGCTGGGCCGCGAGTTCCA |
| | SAKLVVKRREKPASGS | | TGAGCGGGAATACCTGACATACGGGGCCATCCAAAGCGCCAAACTG |
| | KQAAEKPAPEGGGGFN | | GTGGTGAAGCGGCGGGAAAAGCCTGCCTCAGGGTCAAAACAGGCTG |
| | PRIKPTEPKLALATEE | | CGGAGAAACCCGCTCCGGAGGGAGGCGGGGGGTTCAACCCCCGCAT |
| | RFPPIPPEIPSFSAAS | | AAAGCCGACAGAACCAAAGCTGGCTTTAGCCACGGAGGAACGCTTT |
| | KALALQPIPFNAGDYD | | CCCCCAATTCCACCCGAAATCCCAAGTTTTTCCGCGGCCTCTAAAG |
| | FYLHAAFEFNPPKVET | | CGCTGGCCCTTCAGCCCATCCCCTTCAACGCCGGAGACTACGACTT |
| | VTFLGIDRGAAKLGAA | | TTATCTCCATGCCGCCTTCGAGTTCAATCCTCCAAAGGTTGAGACT |
| | TLIDRMGKALETNLDL | | GTAACCTTCCTTGGCATCGACCGAGGCGCGGCCAAGCTGGGAGCGG |
| | DGDAFKAEMRQHEEQI | | CAACGCTGATCGACCGGATGGGCAAGGCCCTCGAAACCAATCTCGA |
| | IRLQKLGKQRSRKFSL | | CCTGGACGGCGACGCGTTCAAGGCGGAGATGAGACAACATGAAGAG |
| | RGKRADMILGEYANRI | | CAAATCATTCGGTTGCAAAAACTCGGAAAGCAACGCTCCCGCAAGT |
| | VAIAKQNRSQIVIEAI | | TCTCTCTGCGCGGCAAGCGCGCCGACATGATCCTGGGCGAGTATGC |
| | KGTTMNLFLKQSQFTK | | CAACCGCATCGTGGCCATCGCCAAACAAAACCGCTCCCAGATCGTC |
| | LKQMLTYKAEREGLPA | | ATCGAGGCCATCAAGGGCACGACGATGAATCTCTTCCTGAAGCAAA |
| | PVEIPAARTSQTCAKC | | GCCAGTTCACCAAGCTGAAGCAGATGCTCACCTACAAAGCCGAGCG |
| | GHWDRANRPKKDAAGK | | GGAGGGTCTTCCTGCCCCGGTCGAGATTCCTGCGGCGCGAACCTCC |
| | AIQDVFLCTACGHRAN | | CAGACCTGCGCGAAGTGTGGACATTGGGATCGCGCGAACCGACCCA |
| | ADSNASLIIALRGLHQ | | AGAAGGACGCGGCGGGAAAGGCGATTCAGGACGTTTTTTTGTGCAC |
| | MENGGKFKKFDLFQQW | | CGCTTGCGGCCACCGCGCCAATGCGGATTCCAATGCCAGCCTGATT |
| | LKELIGRDGSSAFGQG | | ATTGCCCTGCGGGGGCTGCATCAGATGGAAAATGGTGGTAAGTTCA |
| | NQ | | AGAAGTTCGATCTCTTTCAACAGTGGTTGAAGGAGCTTATCGGCCG |
| | | | GGACGGTTCCTCTGCCTTCGGGCAAGGGAACCAGTAG |
| 8 | MFKTARFKVHNPSRHK | 40 | ATGTTCAAGACCGCGCGCTTCAAGGTTCACAACCCGTCAAGACACA |
| | STMLWYAMTRYHETLK | | AGAGCACGATGCTCTGGTACGCCATGACCCGCTATCACGAGACGTT |
| | AVLEETLAIPDLLEQV | | GAAGGCCGTACTCGAAGAGACGCTGGCGATTCCCGATCTGCTGGAA |
| | SELDKKEKRRPNKFKL | | CAAGTCTCCGAGTTGGACAAGAAGGAAAAACGTAGGCCCAACAAAT |
| | SKLLYTIVPKSWELAP | | TCAAGTTAAGCAAACTGCTCTACACAATTGTGCCCAAAAGCTGGGA |
| | LRDYLIGDASAMLMSH | | ACTGGCTCCGCTGCGCGACTATCTCATAGGCGACGCATCCGCGATG |
| | LSKAYKGENESNPPTV | | CTCATGAGCCACCTCAGCAAGGCGTACAAGGGAGAGAACGAGTCTA |
| | SSLAAMTDEEFRKAYS | | ATCCGCCGACGGTCTCAAGCTTGGCCGCAATGACCGACGAGGAGTT |
| | EFSDPEAILAVKPQHQ | | TCGCAAGGCATACAGCGAGTTTTCAGACCCGGAGGCGATACTCGCC |
| | EKIDKASERGETRVAK | | GTCAAGCCGCAGCATCAGGAAAAGATCGACAAGGCGAGCGAACGAG |
| | RLTKIYANWAVSRAAG | | GGGAGACCCGCGTCGCCAAGCGTCTGACCAAAATTTACGCCAACTG |
| | QVLRKLEGALPHPIEF | | GGCCGTCTCACGGGCCGCAGGGCAGGTGCTGCGCAAGCTGGAGGGC |
| | TRNEFGRGCLLAFCDG | | GCGCTGCCCCATCCCATTGAGTTCACCCGCAACGAGTTCGGGCGGG |
| | NYYLLVRLFAQGHRYC | | GCTGCCTGCTTGCTTTTTGCGACGGAAACTACTATCTGCTTGTCCG |
| | KKLVLKDGFIDCKTKA | | GCTTTTCGCCCAAGGCCACCGTTATTGCAAAAAGCTGGTTCTCAAA |
| | PLGGKKYPGLILPLEL | | GACGGCTTTATCGATTGCAAGACGAAGGCGCCACTTGGCGGGAAGA |
| | GREFHEREYLTYGSIQ | | AATACCCCGGCCTGATTCTGCCGCTGGAATTGGGCCGCGAATTCCA |
| | SAKLVVKRREKPASRA | | TGAGCGGGAGTATCTGACTTACGGTTCCATCCAAAGCGCCAAGCTG |
| | KARAAGDALKGGQPVP | | GTGGTGAAGCGGCGGGAAAAGCCTGCATCAAGGGCTAAGGCCCGCG |
| | LNAEDYDFYLHAAFEF | | CCGCTGGCGATGCATTGAAGGGCGGACAGCCCGTCCCCTTAAACGC |
| | NPAKVETETFLGIDRG | | CGAAGATTACGACTTTTATCTCCATGCGGCCTTCGAGTTTAATCCT |
| | AAKLGAATLIDRMGKA | | GCGAAGGTCGAGACGGAGACCTTCCTCGGCATCGACCGTGGCGCGG |
| | LETNLDLDGDAFKLEM | | CCAAGCTGGGCGCGGCAACGCTGATCGACCGGATGGGCAAGGCCCT |
| | RHHEAQIIRLQKLGKQ | | CGAAACCAATCTCGACCTGGATGGCGACGCGTTCAAGTTGGAGATG |
| | RSRRFSLRGKRGEIIL | | AGACACCATGAAGCTCAAATCATTCGGTTGCAAAAACTCGGAAAGC |
| | GEYANRIVSIAKQNRS | | AGCGGTCGCGCAGGTTTTCGCTGCGCGGCAAACGCGGGGAGATCAT |
| | QIVIEAIRGVTMGRFL | | TCTGGGCGAATACGCCAACCGGATAGTGTCGATCGCCAAACAGAAC |
| | KQSQFAKLKQMLTYKA | | CGCTCCCAGATCGTGATCGAGGCCATTCGTGGTGTGACCATGGGCC |
| | EREGLPTPVEVPAAYT | | GGTTCCTGAAGCAAAGCCAGTTCGCCAAGCTCAAGCAGATGCTCAC |
| | SQTCAKCGHKDPANRP | | CTATAAGGCAGAGCGGGAGGGCCTGCCAACCCCGGTCGAGGTTCCT |
| | KKDAAGKAIQDVFKCV | | GCGGCCTATACCTCGCAAACTTGCGCAAAATGCGGTCACAAGGACC |
| | ACGHTANADSNASVII | | CTGCCAACCGTCCCAAGAAAGACGCGGCGGGAAAAGCGATTCAAGA |
| | ALRGLHQVENGGKFKK | | CGTGTTCAAGTGCGTAGCTTGCGGCCACACCGCCAATGCGGACTCC |
| | FDLFQQWLKELIGRDG | | AACGCCAGCGTGATTATTGCGCTGCGGGGGTTGCATCAGGTAGAAA |
| | SVALGQGNQ | | ATGGCGGTAAGTTCAAGAAGTTCGATCTCTTTCAGCAATGGTTGAA |
| | | | GGAGCTTATCGGCCGGGACGGTTCTGTTGCCCTCGGGCAGGGGAAC |
| | | | CAGTAG |
| 9 | MFKTARFKVHNPSRHK | 41 | ATGTTCAAGACTGCGCGTTTCAAGGTCCACAATCCGTCACGGCACA |
| | STMLWYAMTRYHETLK | | AGAGCACAATGCTCTGGTACGCGATGACCCGCTATCACGAGACCTT |
| | DVLEKTLAIPDLLEQV | | GAAGGACGTGCTCGAAAAGACACTGGCGATTCCCGATCTGCTAGAG |
| | SELDKKEKLRPNKYTL | | CAAGTCTCCGAACTGGATAAAAAAGAAAAACTGAGGCCTAACAAAT |
| | AKLVRTIVPKNTGLAS | | ATACTCTTGCTAAACTTGTCCGCACGATAGTCCCCAAGAACACGGG |
| | TVRDYLIGDASAMLMS | | ACTCGCCTCGACTGTGCGCGACTATCTCATCGGCGACGCATCCGCA |
| | HLNKVYKGANESNPPT | | ATGCTGATGAGCCACCTAAACAAAGTCTATAAGGGCGCGAACGAAT |
| | VSSLAAMTDEEFRRAY | | CAAATCCGCCGACGGTCTCTAGCCTCGCTGCAATGACAGATGAGGA |
| | SNFTDPEAKLIIKPQH | | GTTTCGTAGGGCATACAGCAATTTTACGGACCCAGAGGCCAAGCTC |
| | QEKIDKAMEQGETRVA | | ATCATCAAACCGCAGCATCAAGAAAAGATTGACAAGGCAATGGAAC |
| | ERLSKIYANWAISRAA | | AAGGCGAGACACGCGTCGCCGAACGGTTGAGCAAAATCTATGCCAA |
| | GQVLRKLEGTLPHPIE | | TTGGGCCATCTCACGCGCTGCCGGACAAGTGTTGAGAAAGTTAGAA |
| | FTHTEFKRGCLLAFCD | | GGCACTCTGCCCCATCCTATAGAATTCACCCACACCGAATTTAAGC |
| | GKYYALIRLFADKHRY | | GCGGTTGTTTACTCGCATTCTGCGACGGGAAATATTATGCGCTTAT |
| | KQKNFLNSGFIDCKTK | | ACGGCTTTTCGCCGATAAGCATCGTTACAAACAGAAGAATTTTCTC |
| | ESLGGKEYPGLILPLE | | AATTCCGGCTTTATCGATTGCAAGACAAAAGAGTCGCTTGGGGGGA |
| | LGREFHEREYLTHGSI | | AGGAATATCCAGGACTGATTCTGCCGCTTGAACTGGGCCGCGAGTT |
| | QSAKLLVKRRVYSNSQ | | TCATGAGCGTGAGTATCTGACGCACGGATCCATCCAAAGCGCCAAG |
| | TKAPDSKPTTFNAEDY | | TTGTTGGTGAAACGGCGTGTATATTCAAATTCACAGACCAAAGCCC |
| | DFYIHAAFEFQPTKVE | | CGGACTCCAAGCCCACCACCTTCAACGCCGAAGACTATGATTTTTA |
| | TETFLGIDRGAAKIGA | | CATCCACGCTGCCTTCGAGTTTCAACCTACAAAAGTCGAGACAGAA |
| | ATLINLQGKLLETNLD | | ACCTTTCTTGGCATCGACCGGGGCGCTGCGAAGATCGGCGCGGCAA |
| | LEGSAFASEMRRFDEQ | | CCCTGATAAACCTTCAAGGCAAGCTCCTTGAAACCAATCTCGACTT |
| | IKRIQKTGKQRSRKFS | | GGAAGGTTCTGCATTTGCTTCAGAGATGCGGCGCTTTGATGAACAA |
| | IRGKRADIILGEYANR | | ATCAAAAGGATTCAGAAAACAGGGAAACAGCGTTCGCGGAAGTTCT |
| | IVAIAKQYRSQIVIEA | | CAATACGCGGAAAGCGTGCAGACATCATTCTGGGTGAATACGCAAA |
| | IRGVTMGRFLKQSQFT | | TCGCATTGTGGCCATCGCCAAACAATACCGTTCCCAGATTGTCATA |
| | KLKQMLTYKAEREGLP | | GAGGCCATTCGTGGCGTGACGATGGGCCGGTTTCTGAAGCAAAGCC |
| | APVEVPAAYTSQTCAR | | AGTTCACCAAATTGAAGCAGATGCTCACCTACAAGGCAGAGCGCGA |
| | CGHKDAANRPKKDAAG | | GGGTTTGCCAGCCCCTGTTGAAGTTCCTGCTGCTTATACATCGCAG |
| | KAIQDVFLCMACGHKA | | ACCTGCGCACGATGCGGACATAAGGACGCTGCTAATCGCCCCAAGA |
| | NADSNASLIIALRGMH | | AAGATGCGGCAGGAAAAGCGATTCAAGACGTATTCCTGTGTATGGC |
| | QKENGGKLYKKFDLFQ | | TTGCGGTCACAAAGCCAATGCGGATTCCAACGCTAGCCTGATTATT |
| | QWLKELIGRDGSAAPG | | GCTTTGCGGGGTATGCATCAAAAAGAAAATGGTGGTAAGTTATATA |
| | | | AGAAGTTCGATCTCTTTCAACAGTGGTTGAAGGAGCTTATCGGCCG |
| | | | GGACGGTTCCGCTGCCCCAGGGTAG |
| 10 | MFKTARFKVHNPSRHK | 42 | ATGTTCAAAACGGCGCGTTTCAAGGTTCACAATCCGTCACGGCACA |
| | STMLWYAMTRYHETLK | | AGAGCACTATGCTCTGGTATGCCATGACCCGCTATCACGAGACTTT |
| | DVLEKTLAIPDLLEQI | | GAAGGACGTACTCGAAAAGACACTGGCGATTCCAGATCTGCTAGAA |
| | SELDKKEKLRPNQYKL | | CAAATCTCAGAACTGGATAAGAAAGAAAAACTACGTCCTAACCAGT |
| | RMLLRKILPRGWELAP | | ACAAGCTCAGGATGCTCCTCCGAAAAATTCTTCCTAGAGGTTGGGA |
| | LRGYLELDASAMLMSH | | GCTTGCACCGCTACGCGGATATCTTGAACTTGATGCGTCAGCGATG |
| | FNKAYKGANGSNPPTV | | CTGATGAGTCACTTCAATAAAGCATACAAGGGGGCAAACGGATCCA |
| | SSLDPMTEKEYHEAYK | | ATCCGCCGACAGTTTCCAGTCTTGACCCTATGACCGAGAAAGAATA |
| | EFTNPEASLSIKPQQQ | | TCACGAGGCTTACAAAGAATTTACAAACCCGGAAGCTAGTCTTTCC |
| | EKINAASERGETRVAK | | ATTAAGCCACAGCAACAGGAAAAGATTAACGCGGCAAGTGAACGAG |
| | RLSKIYANWAISRAAG | | GTGAAACTCGCGTCGCCAAACGCCTGAGCAAAATCTACGCCAATTG |
| | LVLRKLEGTLPHPIEF | | GGCCATCTCCCGCGCCGCAGGTCTTGTTCTCAGAAAGTTAGAAGGC |
| | TNNEFGRGCLLAYCDG | | ACTCTACCACATCCCATCGAATTTACCAACAACGAGTTTGGGCGGG |
| | NYYLLVRLFAQGNHYC | | GCTGCCTACTCGCCTATTGCGACGGAAACTACTATCTGCTCGTCCG |
| | KKLVLKDGFIDCKTKE | | ACTTTTCGCACAAGGCAACCATTATTGTAAGAAACTTGTACTGAAA |
| | LLAGKKYPGLILPLEL | | GATGGCTTCATTGATTGCAAGACGAAAGAGCTACTTGCTGGAAAGA |
| | GREFHEQEYLTYGSIQ | | AATATCCGGGGCTGATTCTACCGCTAGAACTGGGACGCGAATTCCA |
| | SAKLIVKRREKPTSAA | | TGAGCAGGAATATCTTACCTACGGTTCTATCCAGAGCGCCAAGCTG |
| | KARIHNTGLAARLKSC | | ATTGTGAAACGGCGAGAGAAGCCTACCTCAGCGGCTAAAGCCCGCA |
| | PDTKQTASFNAEDYDF | | TTCATAACACAGGATTGGCGGCACGATTGAAGTCGTGCCCTGATAC |
| | YVHAAFEFQPTQIETE | | AAAGCAAACAGCCTCCTTCAACGCGGAAGACTACGATTTTTATGTT |
| | TFLGIDRGAAKIGAAT | | CATGCGGCCTTCGAGTTTCAACCGACACAGATCGAGACAGAAACCT |
| | LIDREGKPIETNLDLE | | TCCTTGGCATTGATCGCGGCGCGGCGAAGATAGGCGCGGCAACTTT |
| | GSAFASEMRRYEEQIK | | GATTGACCGTGAAGGCAAGCCGATTGAAACTAATCTCGACCTTGAA |
| | RIQKTGKQRSRKFSLR | | GGCTCGGCATTTGCTTCTGAAATGCGGCGCTATGAAGAACAAATCA |
| | GKRADIILGEYANRIV | | AGCGGATACAGAAGACAGGCAAGCAACGATCTCGCAAGTTTTCACT |
| | AIAKEYRSQIVIEAIR | | GCGCGGAAAGCGCGCGGACATCATTCTGGGTGAATACGCGAATCGG |
| | GVTMGRFLKQSQFAKL | | ATTGTGGCTATCGCCAAGGAATACCGCTCCCAGATTGTGATTGAGG |
| | KQMLTYKAEREGLPAP | | CCATTCGAGGCGTGACGATGGGACGGTTTCTGAAGCAAAGCCAGTT |
| | VEVPAARTSQTCARCG | | CGCCAAGCTGAAACAAATGCTCACCTACAAAGCAGAGCGCGAGGGA |
| | HWDRANRPKKDAAGKA | | TTACCTGCGCCGGTCGAAGTTCCGGCGGCGCGCACCTCGCAAACCT |
| | IQDVFLCVACGHKANA | | GCGCACGATGCGGACATTGGGACAGAGCCAATCGACCCAAGAAGGA |
| | DSNASLIIALRGMHQK | | CGCGGCGGGAAAAGCGATTCAGGACGTGTTTCTGTGCGTGGCCTGC |
| | ENGGKFKKFDLFQLWL | | GGTCACAAGGCCAATGCGGATTCCAATGCGAGCCTGATTATTGCCT |
| | KELIGRDGSIASGQGN | | TGCGAGGGATGCATCAAAAAGAAAATGGTGGTAAGTTCAAGAAGTT |
| | Q | | CGATCTCTTTCAACTGTGGTTGAAGGAGCTTATCGGCCGGGACGGT |
| | | | TCCATTGCCTCAGGGCAGGGGAACCAGTAG |
| 11 | MFKTARFKVHNPSRHK | 43 | ATGTTCAAAACCGCGCGCTTCAAAGTTCACAATCCGTCCCGACACA |
| | STMLWYAMTHYHDALK | | AGAGCACAATGCTCTGGTACGCCATGACCCACTATCACGATGCGTT |
| | AVLEKTLAVPDLLEQI | | GAAGGCCGTGCTCGAAAAGACGCTGGCTGTTCCCGATCTGCTGGAG |
| | SELDKKEKLRPNQYKL | | CAAATCTCAGAACTTGACAAAAAAGAAAAACTTCGCCCTAACCAGT |
| | RKLLRTILPRGWELAP | | ACAAGCTCAGGAAGCTGCTTCGTACGATTCTTCCGAGGGGTTGGGA |
| | LTRYLELDAAAMLMSH | | ACTTGCACCTTTGACACGCTATCTCGAATTGGATGCAGCTGCGATG |
| | FNKSYKGANESNPPTV | | CTGATGAGCCATTTCAATAAGTCGTACAAGGGAGCGAACGAGTCCA |
| | SSLDAMTDAEFRKAYS | | ATCCGCCCACAGTCTCCAGCCTGGATGCGATGACAGACGCGGAGTT |
| | EFTDPEAKLTVKPQHQ | | TCGCAAGGCATACAGCGAGTTCACAGACCCGGAGGCAAAGCTCACC |
| | EKIDKAKERGETRVAK | | GTCAAGCCGCAGCATCAGGAGAAGATCGACAAAGCAAAGGAACGAG |
| | RLSKIYASWAVSRAAG | | GCGAAACTCGCGTCGCCAAACGTCTGAGCAAAATCTACGCCAGCTG |
| | QVLKKLEGALPHPIEF | | GGCCGTCTCCCGCGCCGCAGGACAAGTTCTGAAAAAGTTAGAAGGC |
| | TNNEFGQGCLLAYCDG | | GCTCTGCCCCACCCCATCGAATTCACCAACAATGAGTTCGGGCAAG |
| | NYYLLVRLFAKKHRYK | | GCTGCTTACTCGCCTATTGCGATGGAAACTACTATCTGCTCGTCAG |
| | QNNVLKDGFISCKTKE | | GCTCTTCGCCAAAAAACACAGGTACAAGCAGAATAATGTACTCAAG |
| | PLGGKKYPGLILPLEL | | GACGGCTTCATCAGCTGTAAAACGAAAGAGCCTCTTGGCGGGAAGA |
| | GREFHEQEYLTYGSIQ | | AATACCCCGGCCTGATTCTGCCTCTTGAATTGGGTCGAGAATTCCA |
| | SAKLVVKRREKPASRA | | TGAACAGGAGTACCTGACTTACGGCTCCATCCAAAGCGCCAAGCTG |
| | KARTSSRISTDKQKPI | | GTGGTGAAGCGGCGGGAAAAGCCTGCATCGAGGGCTAAGGCCCGCA |
| | PFNAEDYDFYVHAAFE | | CCTCTTCTCGCATCTCAACGGACAAGCAGAAGCCCATCCCCTTCAA |
| | FNPPKVETMTFLGIDR | | TGCTGAAGACTACGATTTTTACGTTCATGCGGCCTTCGAGTTTAAC |
| | GAAKLGAATLIDQHGK | | CCACCGAAGGTTGAAACTATGACTTTCCTCGGTATCGACCGCGGAG |
| | RLETNLDLDGSAFAAE | | CTGCAAAACTAGGAGCAGCGACGCTGATCGACCAGCACGGCAAGCG |
| | MRRYEQQIKRIQQQGK | | TCTGGAAACCAATCTCGATCTGGACGGCTCAGCATTCGCTGCGGAG |
| | QKSRKFSLRGKRAEII | | ATGCGGCGCTACGAACAGCAGATCAAGCGAATTCAGCAACAGGGAA |
| | LGEYANRIVAIAKENR | | AACAGAAGTCGCGCAAGTTTTCCCTGCGCGGCAAACGAGCAGAAAT |
| | SQIVIEAIKGVTMGRF | | CATTCTGGGAGAGTACGCCAACCGGATTGTTGCTATTGCCAAAGAG |
| | LKQSQFTKLKQMLTYK | | AATCGCTCACAAATTGTCATCGAAGCCATCAAGGGCGTGACGATGG |
| | AEREGLPAPIEVPAAF | | GCCGATTCCTCAAGCAAAGCCAGTTCACCAAATTGAAGCAGATGCT |
| | TSQTCARCSHKDPANR | | TACCTACAAGGCGGAGCGAGAGGGATTGCCAGCCCCTATCGAAGTT |
| | PKKDAAGKSIQDVFLC | | CCTGCGGCGTTCACTTCACAGACCTGCGCCAGATGCAGCCATAAAG |
| | TACGHHANADSNASLI | | ATCCAGCCAATCGTCCCAAGAAAGATGCGGCTGGAAAGTCGATTCA |
| | IALRGMHQIENGGKFK | | GGATGTGTTTTTGTGCACGGCTTGCGGTCATCACGCCAACGCAGAT |
| | KFDIFQQWLKELIGRD | | TCCAATGCCAGTCTGATTATTGCATTGCGGGGGATGCATCAGATTG |
| | GSSAPGQLNP | | AAAATGGTGGTAAATTCAAGAAGTTCGATATCTTTCAACAGTGGTT |
| | | | GAAGGAGCTTATCGGCCGGGACGGTTCCTCTGCCCCAGGGCAGTTG |
| | | | AACCCGTAG |
| 12 | MKDIKHSLISGQKAQK | 44 | ATGAAAGATATCAAACATTCACTTATAAGTGGACAAAAAGCTCAAA |
| | FRKHLAMSSASASVKK | | AGTTCAGAAAGCATTTGGCAATGTCATCTGCCAGCGCATCAGTCAA |
| | NKTSILTYKFRLDLNE | | GAAAAATAAAACATCGATTTTGACATATAAATTCAGACTTGATTTA |
| | NMSEIQDRIPKFSDYI | | AATGAAAATATGTCAGAAATACAAGACAGAATACCTAAGTTTTCAG |
| | KLYNKIEGVEPGTLTH | | ACTACATAAAATTATATAATAAAATTGAAGGTGTAGAGCCTGGTAC |
| | YLCTFVLAGFRLFSNA | | ATTAACACATTATTTATGCACCTTTGTCCTGGCTGGATTTAGACTT |
| | KSAFEFIKSQNNPCLE | | TTTTCCAATGCAAAATGAGCATTTGAATTTATAAAATCACAAAATA |
| | HLSSHKLLKSSAVAFD | | ACCCATGTTTAGAGCACTTATCCTCACATAAACTTTTAAAATCCAG |
| | LTANLAISEPGYEPYL | | CGCTGTTGCTTTTGATCTTACAGCAAATCTTGCTATCTCAGAGCCT |
| | AIARILERYTDPDKKI | | GGCTATGAGCCATATTTGGCAATTGCCAGAATTCTTGAGAGATACA |
| | NSFVKDNFTTYNNNAL | | CCGATCCGGACAAGAAAATTAACAGCTTTGTAAAAGACAACTTTAC |
| | SWLLGKGHKFFKESTA | | AACATATAACAACAATGCTCTATCCTGGCTTTTGGGTAAAGGTCAT |
| | QEIALYYGIPDYKFDC | | AAATTTTTTAAAGAATCTACAGCGCAAGAAATAGCTTTGTATTATG |
| | AKAIKNAADKLEFNSS | | GCATACCAGATTATAAGTTTGACTGTGCCAAGGCCATTAAAAATGC |
| | LFSNDMRLSQFRSCFG | | AGCTGACAAATTAGAGTTCAACAGTTCACTATTTTCAAATGACATG |
| | GHIDSWATNYIKRLLE | | CGTCTTTCTCAATTCAGATCATGCTTTGGAGGACATATTGACAGCT |
| | LEKIIANISYEIKIPK | | GGGCAACTAACTATATAAAAAGGCTTTTGGAGCTTGAAAAAATCAT |
| | AFISSSNDFLTHCNLN | | AGCAAACATTAGCTATGAAATCAAAATACCTAAAGCTTTTATCTCC |
| | RDDIEELISNIKSSST | | TCTTCCAATGACTTTTTAACACATTGTAATCTTAACCGCGACGATA |
| | ITDVKDALSTLLGHKQ | | TTGAAGAATTAATTTCTAATATAAAAAGCAGTTCAACCATTACTGA |
| | GASSADIKAIRDYSEL | | TGTCAAAGATGCTCTTAGCACATTACTGGGACACAAACAAGGCGCA |
| | INRLCAYKEQIFNTID | | TCATCTGCCGATATAAAGGCAATAAGAGATTACTCAGAGCTAATCA |
| | QAAEDKNSLWHDIRRQ | | ACAGACTTTGTGCTTATAAAGAGCAGATATTTAATACAATAGATCA |
| | TKDELQTWEKLEKLPK | | GGCCGCTGAAGACAAAAATTCATTGTGGCATGACATAAGACGCCAA |
| | LNDLSGGVPQAENELN | | ACAAAAGATGAATTGCAGACTTGGGAGAAATTAGAAAAACTGCCAA |
| | AKLMQLKLVTEAQNNH | | AACTAAATGATTTAAGTGGCGGAGTTCCGCAGGCTGAGAATGAATT |
| | FAKIMQWVHSNIKDFS | | AAACGCAAAACTAATGCAGCTTAAACTTGTAACAGAGGCACAGAAC |
| | PFNHIVQTEQEKLDNR | | AATCATTTTGCAAAGATTATGCAGTGGGTGCATAGCAATATTAAAG |
| | PKENTTACDLAVRMFL | | ACTTTTCTCCCTTTAATCATATAGTGCAGACTGAGCAGGAAAAATT |
| | HKVGRIAREDNNNLCK | | AGATAACAGACCAAAGGAAAATACAACTGCCTGCGATCTTGCCGTG |
| | ELQQWFLDNKVFDNKT | | AGAATGTTCCTGCATAAAGTCGGACGTATAGCCCGCGAAGATAACA |
| | DFNKYFHNKLGSIYIS | | ACAATCTATGTAAAGAGTTGCAGCAATGGTTTTTAGATAATAAAGT |
| | PYSTQKNAGYKINKEV | | TTTTGATAATAAGACTGATTTTAATAAATACTTTCATAATAAGCTT |
| | LNFGEKIVLLFTDKLQ | | GGCTCTATATACATATCCCCTTACAGCACTCAGAAAAATGCAGGAT |
| | EINKRYEGNSIAEKSE | | ATAAAATAAATAAAGAAGTGTTAAACTTTGGTGAAAAAATTGTCTT |
| | LNSLLKLNYFYYNFFI | | GCTTTTCACTGACAAATTACAAGAGATTAATAAGAGGTATGAAGGT |
| | SGINKAVPVSIVKPLL | | AATTCTATTGCTGAAAAATCAGAGTTAAATTCACTTTTAAAATTAA |
| | PDDMLEQSLSATHKIR | | ACTATTTTTACTATAACTTCTTTATATCTGGCATAAATAAAGCAGT |
| | LKSNEVDPSSLSSIFN | | TCCCGTTTCTATTGTCAAACCGCTACTGCCAGATGATATGCTAGAG |
| | IYKSLISGCYTVLNRE | | CAATCTCTATCTGCAACACACAAAATAAGATTAAAAAGCAATGAAG |
| | TFFLRTKFSWIENFTL | | TGGATCCTTCTTCCCTAAGCTCCATTTTTAATATTTATAAATCATT |
| | FYVPKADASWIMPKRY | | AATAAGTGGTTGCTATACAGTATTAAATCGTGAAACTTTCTTCTTA |
| | LKNTRWQQYIEEEVLV | | AGAACAAAGTTTTCCTGGATTGAAAACTTCACCCTGTTCTATGTTC |
| | FENDKYKVDITQTFNN | | CAAAGGCAGATGCCTCGTGGATAATGCCTAAGAGATACCTAAAAAA |
| | ICSAPADYAELLVQLP | | TACACGCTGGCAGCAGTATATAGAAGAGGAGGTTTTAGTTTTTGAA |
| | HDWFYQLPYECAKEDN | | AATGATAAATATAAGGTAGATATAACTCAGACATTTAATAATATAT |
| | YVQALAICKDKGFPKQ | | GCTCTGCGCCTGCTGACTATGCTGAGCTTCTTGTTCAATTACCTCA |
| | SRLNTHISGRLIGPSS | | TGACTGGTTTTATCAATTGCCATATGAATGTGCCAAGGAAGACAAT |
| | FKSKLDSVLIYNGDVT | | TATGTACAGGCCTTAGCAATATGTAAAGATAAAGGATTCCCTAAGC |
| | ISDMTLLVEQRVSQQL | | AGAGCAGATTAAATACGCACATATCTGGCAGACTTATAGGTCCATC |
| | KPDESLELKKYDPEFT | | ATCATTTAAGTCAAAACTAGACTCTGTACTTATATATAACGGCGAT |
| | LAIPINDARSQSTNYS | | GTCACTATTTCTGATATGACGCTGCTTGTTGAGCAGAGAGTCAGTC |
| | FKHIIAIDQGEIGPSY | | AGCAGCTAAAGCCCGATGAGAGTCTTGAGCTTAAAAAGTATGATCC |
| | AVFNLSDAGNANAEPI | | TGAATTTACACTTGCAATCCCTATTAATGATGCAAGATCTCAGAGC |
| | ATGSIRIPSIRRLIKS | | ACCAATTACAGCTTTAAACACATAATTGCAATTGATCAAGGAGAGA |
| | VSSFRKKKSTTQKFNQ | | TAGGACCTTCATATGCAGTGTTTAATTTAAGCGATGCCGGCAATGC |
| | RFDSTMFNIRENVTGD | | CAATGCAGAGCCTATTGCAACTGGCTCAATAAGAATACCTTCAATC |
| | ICSVIVGLMQKYNAFP | | AGGCGCCTTATAAAATCAGTTTCTTCTTTTAGAAAGAAAAAGAGTA |
| | VLEREVSNLESGSKQL | | CAACGCAGAAATTTAATCAGCGCTTTGATTCTACAATGTTTAACAT |
| | SLVYKAVNSMFLYSDV | | CAGAGAAAATGTCACAGGCGATATCTGCAGTGTCATTGTAGGTCTT |
| | EMQNTNRKSWWKNADH | | ATGCAAAAATATAATGCTTTTCCAGTGCTGGAACGAGAAGTTTCTA |
| | WQTNILRLIRGENKTS | | ACCTTGAGAGCGGTTCAAAGCAATTATCATTAGTATATAAAGCTGT |
| | KSVKLNGQNYKELKIY | | AAACTCAATGTTCCTATACTCTGATGTAGAAATGCAAAATACAAAT |
| | PGVSVSAYMTSRICSC | | CGTAAATCATGGTGGAAAAATGCAGATCACTGGCAAACTAATATTT |
| | CGRNIFELIKNDELED | | TAAGGCTTATAAGGGGTGAGAATAAAACCTCAAAATCTGTAAAACT |
| | KHKKYQVNAQGEINIR | | CAATGGTCAGAACTATAAAGAATTAAAAATCTACCCTGGAGTGAGT |
| | GEVIKLYQKSDSHKTL | | GTCAGTGCATATATGACCAGCAGAATCTGCTCATGCTGTGGCAGAA |
| | VPGLKSKKTYNAINQR | | ATATTTTTGAATTAATAAAAAATGATGAGCTTGAGGATAAACATAA |
| | APMVTPYPEGIIDIEQ | | AAAATATCAGGTTAACGCACAGGGCGAGATCAATATAAGAGGTGAA |
| | LKKIIRFNLRRAPASR | | GTAATTAAACTCTATCAAAAATCAGACAGTCACAAAACTTTAGTAC |
| | MSKDSTQSRYFCVFKN | | CTGGACTTAAAAGTAAAAAAACCTATAACGCCATCAATCAAAGGGC |
| | CKNHQVEKHADINAAI | | CCCTATGGTTACCCCATACCCTGAGGGCATTATAGATATTGAGCAG |
| | NIGRRFLTDIIIHN | | CTAAAAAAAATAATTCGCTTTAATTTAAGGCGCGCTCCGGCAAGTC |
| | | | GCATGTCTAAAGATAGTACTCAAAGCAGATATTTCTGTGTGTTTAA |
| | | | GAACTGTAAAAATCATCAGGTTGAAAAACATGCAGATATTAATGCA |
| | | | GCGATAAATATCGGCAGAAGATTTTTAACTGATATTATTATTCATA |
| | | | ATTAA |
| 13 | MLTTKFKLELPAGCPL | 45 | ATGCTGACGACCAAGTTCAAACTCGAGCTTCCCGCGGGATGTCCGC |
| | REDAATFDECRKLYDV | | TTCGTGAGGATGCCGCCACGTTTGACGAGTGCCGGAAACTCTATGA |
| | VEGCGNGTLTGFLFSV | | CGTGGTGGAAGGTTGCGGAAACGGTACGCTCACCGGTTTTCTTTTT |
| | ILSGFRIFPDGKMAEI | | TCCGTCATTCTTTCCGGCTTTCGGATTTTTCCGGACGGCAAGATGG |
| | FANRSVYDEDEFRSAL | | CCGAGATTTTCGCCAACCGTTCCGTCTACGACGAGGACGAATTCCG |
| | VEAVGAPLPRFTVKAL | | GAGTGCTCTGGTCGAGGCGGTGGGTGCGCCTTTGCCCCGATTTACC |
| | IKRLQMEVRARGNKDN | | GTCAAGGCGCTCATCAAGCGACTTCAGATGGAGGTGCGGGCTCGCG |
| | RFVAEVMMKEYRQTLC | | GCAACAAGGACAACCGCTTCGTTGCCGAAGTCATGATGAAGGAATA |
| | GKTLPKGVDESYVDRL | | TCGGCAGACCCTCTGCGGTAAGACCCTCCCCAAAGGAGTCGATGAA |
| | FEEMARELTSRYRSWN | | TCCTACGTCGACAGGCTCTTTGAAGAAATGGCCCGGGAGCTGACTT |
| | ELKGDLLGACKAVDAA | | CGCGCTACCGCTCCTGGAACGAACTCAAGGGCGATCTGCTCGGCGC |
| | LRGFGDFPSLATMVTR | | TTGTAAAGCGGTGGACGCGGCGCTCCGAGGCTTCGGAGATTTTCCT |
| | AAARRLPKDSTIVFDP | | TCTCTGGCAACGATGGTGACGCGGGCCGCTGCACGTAGATTGCCGA |
| | QSPFIDVQTIGVDAMP | | AGGATTCGACAATCGTCTTCGATCCTCAATCGCCTTTCATTGACGT |
| | YAAVATLLSYPESVGE | | GCAAACGATCGGGGTCGATGCCATGCCCTACGCGGCAGTGGCGACA |
| | KRRDFVQNHLTTPSAA | | CTTCTTTCGTATCCCGAAAGTGTCGGTGAAAAGAGACGGGATTTCG |
| | GLSWLFNRGLELFSEE | | TCCAAAATCACCTTACGACGCCCTCGGCGGCGGGCCTGAGTTGGCT |
| | SVEELCRLFHVPEDQR | | ATTCAATCGGGGACTGGAACTTTTTTCGGAGGAAAGCGTCGAAGAA |
| | TRIVQIQNAARATPRQ | | CTTTGTCGGCTCTTTCATGTGCCGGAAGATCAACGGACCCGCATAG |
| | SFFLKKGGAPLGYHDF | | TTCAAATTCAGAATGCGGCCCGAGCGACCCCGAGGCAAAGCTTTTT |
| | RSAFAGRINSWTANYL | | CCTGAAGAAGGGCGGCGCTCCCCTCGGATACCACGATTTTCGAAGC |
| | NRLEELQGLLHDLTDE | | GCCTTTGCCGGCCGCATCAACAGTTGGACGGCAAACTACCTCAACC |
| | LRLPDLVRNGEDFLAT | | GTCTTGAAGAACTGCAGGGACTTCTTCACGATCTGACGGACGAACT |
| | TDCRREEVEILCRSFS | | TCGGTTGCCCGACCTTGTCCGAAACGGCGAGGACTTTCTCGCAACA |
| | RERDRAQTAVEHLIGA | | ACCGACTGCCGCCGGGAAGAGGTGGAGATTTTGTGCCGAAGCTTTT |
| | DPLQVVSDVAAIEEYS | | CTCGGGAGCGCGATCGTGCGCAGACGGCTGTGGAGCATCTCATCGG |
| | RIVNRLCAIKEQIVNS | | CGCCGATCCCTTGCAGGTGGTGAGCGACGTTGCGGCGATCGAGGAA |
| | LRQAEDDKASRWTTLW | | TACAGTCGAATCGTCAACCGCCTCTGCGCCATCAAGGAGCAGATCG |
| | SEVKNEFQPWEKLIRL | | TCAATTCGCTCCGACAGGCCGAGGACGACAAGGCTTCCCGTTGGAC |
| | PKLNGMSGGVPPAQDE | | TACGCTCTGGTCCGAGGTCAAAAACGAATTTCAACCGTGGGAGAAA |
| | LETILARYSDVVRGAT | | CTTATCCGTCTTCCTAAACTCAACGGAATGTCGGGCGGAGTGCCTC |
| | EHFDVVMEWAAKTGAE | | CTGCGCAGGACGAACTCGAGACGATTCTTGCCCGCTATTCTGACGT |
| | GDILKKFAETEQQRAD | | AGTCCGAGGTGCAACCGAACATTTTGATGTCGTCATGGAGTGGGCG |
| | QRAPGKYDGRELALRL | | GCCAAAACGGGCGCCGAGGGCGATATCCTCAAAAAGTTTGCCGAGA |
| | VLQRVARVVRDRSDVC | | CGGAACAGCAGCGTGCCGATCAACGCGCTCCGGGCAAATACGACGG |
| | AENVRQWFLKENIFAE | | TCGGGAGCTGGCGTTGCGTCTCGTCCTGCAGCGCGTCGCCCGTGTC |
| | RKDFNKFFFNRLGSLY | | GTGCGCGATCGTTCCGACGTCTGCGCCGAGAATGTTCGGCAGTGGT |
| | VSPFSNRRHVGYKLSD | | TCCTGAAGGAAAACATCTTTGCGGAGCGGAAGGACTTCAACAAGTT |
| | GLVERSGAVWRELLAL | | TTTCTTCAATCGTCTTGGAAGCCTCTATGTTTCGCCCTTCAGCAAC |
| | VKEKRGAYEAFSEAGE | | CGCCGTCACGTCGGCTACAAATTGTCCGACGGACTCGTGGAGCGGT |
| | TFLRLENLLMVMRIGA | | CCGGCGCCGTTTGGCGCGAACTGCTCGCCTTGGTGAAGGAGAAGAG |
| | LTENVPAEVAALRLDE | | GGGAGCCTATGAAGCGTTCTCGGAAGCGGGCGAGACGTTCCTGCGT |
| | ETALESVSEGLKLQLQ | | CTTGAAAACCTCTTGATGGTTATGAGAATCGGCGCTCTCACGGAGA |
| | QAEVPPSVLAKAFNVY | | ATGTTCCTGCGGAAGTCGCCGCTCTTCGTCTCGACGAAGAGACAGC |
| | VSLLSGCLIALRRERF | | GCTCGAGAGCGTTTCCGAAGGTCTGAAGCTTCAGCTCCAACAAGCC |
| | FLRTKFSFVGNTALVY | | GAAGTGCCGCCCTCGGTACTTGCCAAGGCCTTCAACGTCTATGTCA |
| | VPKEKSWPMPSRYEAS | | GTCTGCTGAGCGGTTGCTTGATCGCCCTGCGGCGCGAACGCTTTTT |
| | PSWTPIFENDVLVRLS | | CCTTCGCACCAAGTTCTCCTTCGTCGGCAACACGGCGCTCGTTTAC |
| | TGEVEVAETFRRAAAL | | GTTCCGAAGGAAAAGAGCTGGCCGATGCCCTCGCGCTACGAAGCAT |
| | WGRTTDPVLKKALREL | | CGCCCTCCTGGACTCCGATTTTCGAAAACGACGTGTTGGTGCGGCT |
| | FHQLPHDWCCQVSVRS | | CTCGACCGGGGAAGTGGAGGTTGCCGAAACCTTCCGTCGCGCCGCT |
| | SGDMTPAKRKEDDRDV | | GCGCTCTGGGGACGAACGACGGATCCCGTCCTGAAGAAAGCTTTGC |
| | LIVEKKGKYDSTIISK | | GGGAACTCTTTCATCAGTTGCCGCACGACTGGTGTTGTCAGGTCTC |
| | KIAATALVRLVGPSTH | | TGTTCGGAGTTCCGGCGACATGACGCCGGCAAAGCGGAAAGAGGAT |
| | KERLNRLLLDVGEVAC | | GATCGGGACGTGTTGATCGTGGAGAAGAAAGGAAAGTACGACTCTA |
| | DMTLLADQEILQKVED | | CGATCATCTCGAAAAAGATTGCGGCCACGGCTCTTGTGCGCCTTGT |
| | GRVILSLGKLQFSLSV | | CGGACCGAGCACCCATAAAGAACGGCTCAACCGTTTGCTGTTGGAT |
| | PISTPAEQSEDEVKSE | | GTTGGAGAAGTGGCCTGCGACATGACGTTGCTTGCCGACCAGGAGA |
| | RKSTHFRRIVAIDQGE | | TCCTTCAGAAAGTGGAGGACGGTCGTGTAATTTTGTCTTTGGGAAA |
| | RGFAFAVFRLEDAGKE | | ACTGCAGTTTTCACTTTCCGTGCCGATTTCAACTCCGGCCGAACAA |
| | GAQPIAQGFVNIPSIR | | AGCGAGGACGAAGTGAAGAGCGAGAGAAAATCGACGCACTTCCGAA |
| | RLIARVHSYRKGKQSV | | GAATCGTCGCCATCGACCAAGGGGAACGAGGATTTGCCTTCGCCGT |
| | QKFSQRFDSTMFTLRE | | ATTCCGATTGGAAGACGCGGGAAAGGAGGGTGCGCAGCCGATTGCT |
| | NVAGDVCGAIAGLMCR | | CAGGGATTCGTGAACATTCCGTCCATTCGCCGTCTTATCGCCCGCG |
| | YRAIPVLERQVSNLAS | | TGCACTCCTACCGCAAGGGCAAGCAGTCCGTACAAAAGTTCAGTCA |
| | GGKQLELVYKMVNARF | | GCGCTTTGACTCCACGATGTTTACCCTGCGCGAAAACGTGGCGGGC |
| | LDDRIPMHSLERTSWW | | GACGTGTGCGGAGCCATCGCCGGTCTCATGTGCCGCTACCGCGCCA |
| | CGTSDWVIPDLWVEVP | | TTCCGGTGCTCGAACGGCAAGTGAGCAATCTCGCGAGCGGCGGCAA |
| | ESYAVKAKKDEILKKD | | GCAGCTTGAACTCGTCTACAAGATGGTCAACGCCCGTTTCCTGGAC |
| | GKFYRTLRITPGVGVN | | GACCGCATTCCGATGCACAGTCTTGAGCGCACTTCCTGGTGGTGCG |
| | AKWTSRICSQCGGNAM | | GAACCTCCGATTGGGTCATCCCTGATTTGTGGGTCGAAGTTCCCGA |
| | ELIEKAREEKVKTVTL | | AAGTTATGCCGTCAAGGCAAAGAAGGATGAGATTCTCAAGAAGGAC |
| | DANGEVTLFGRTVRLY | | GGGAAGTTTTACCGAACCCTGCGGATTACGCCGGGGGTGGGCGTGA |
| | KRPSEERSREARRRNE | | ACGCCAAGTGGACGAGCCGCATCTGTTCGCAGTGCGGCGGCAATGC |
| | RAPWTEPRANVKLSLD | | GATGGAACTGATCGAGAAGGCTCGTGAAGAAAAGGTAAAGACCGTA |
| | DFRRAVAENMRRQPKS | | ACGCTCGACGCCAACGGAGAAGTCACGCTCTTTGGCAGGACAGTGC |
| | LQSRDTSQSRYFCVFT | | GTCTTTATAAGAGGCCTTCGGAAGAACGAAGCAGGGAAGCCAGGCG |
| | DCRCHNKEQHADINAA | | CCGCAACGAGCGTGCCCCCTGGACGGAGCCGCGTGCAAACGTCAAG |
| | VNIGRRFLESLLRE | | CTTTCTCTCGACGACTTCCGAAGAGCTGTCGCCGAAAACATGCGTC |
| | | | GCCAGCCCAAGAGCCTTCAGAGCCGAGACACGTCGCAGAGTCGCTA |
| | | | CTTCTGTGTTTTCACAGATTGCCGCTGCCATAACAAGGAACAGCAC |
| | | | GCGGACATCAACGCAGCAGTCAACATCGGACGCCGTTTTCTGGAGA |
| | | | GCCTGTTGCGCGAGTGA |
| 14 | MQTKKTHLHLISAKAS | 46 | ATGCAAACAAAGAAAACACACCTTCATCTAATTTCAGCCAAGGCAT |
| | RKYRRTIACLSDTAKK | | CAAGAAAATATCGAAGAACGATTGCATGCTTATCTGATACAGCAAA |
| | DLERRKQSGAADPAQE | | AAAAGATCTGGAACGACGTAAGCAGTCGGGAGCGGCTGATCCCGCT |
| | LSCLKTIKFKLEVPEG | | CAGGAACTCAGTTGCTTGAAAACAATTAAATTTAAGTTAGAGGTTC |
| | SKLPSFDRISQIYNAL | | CTGAAGGGTCGAAGCTGCCGTCTTTTGACAGAATTTCGCAAATTTA |
| | ETIEKGSLSYLLFALI | | CAATGCCCTCGAGACAATAGAGAAGGGCTCCCTGTCATATCTTCTT |
| | LSGFRIFPNSSAAKTF | | TTTGCGTTAATTCTCTCTGGATTCAGAATTTTTCCAAACTCGTCTG |
| | ASSSCYKNDQFASQIK | | CAGCAAAGACGTTTGCAAGCTCCTCTTGTTATAAAAACGATCAATT |
| | EIFGEMVKNFIPSELE | | TGCATCCCAGATAAAAGAAATTTTCGGGGAAATGGTGAAAAATTTT |
| | SILKKGRRKNNKDWTE | | ATTCCTTCGGAACTTGAGAGTATTCTCAAAAAAGGCCGCAGAAAAA |
| | ENIKRVLNSEFGRKNS | | ACAATAAAGATTGGACAGAGGAGAATATTAAGAGAGTCCTGAATAG |
| | EGSSALFDSFLSKFSQ | | CGAATTTGGTAGGAAAAATTCGGAAGGGTCTTCGGCCTTGTTTGAT |
| | ELFRKFDSWNEVNKKY | | TCCTTTTTAAGCAAATTTTCACAAGAATTGTTCCGGAAATTTGATT |
| | LEAAELLDSMLASYGP | | CATGGAACGAGGTCAATAAGAAATATCTGGAGGCTGCGGAATTGCT |
| | FDSVCKMIGDSDSRNS | | GGATAGCATGCTCGCCTCATACGGACCATTTGATTCCGTTTGCAAG |
| | LPDKSTIAFTNNAEIT | | ATGATTGGAGACTCTGATTCTAGAAACTCGTTGCCAGACAAAAGTA |
| | VDIESSVMPYMAIAAL | | CGATTGCTTTTACAAACAATGCTGAGATCACAGTAGACATCGAATC |
| | LREYRQSKSKAAPVAY | | ATCAGTAATGCCCTATATGGCAATTGCGGCTTTGTTAAGAGAATAC |
| | VQSHLTTTNGNGLSWE | | CGTCAAAGCAAATCAAAAGCAGCACCGGTTGCGTATGTTCAAAGCC |
| | FKFGLDLIRKAPVSSK | | ACTTAACAACGACGAATGGCAATGGCTTGTCATGGTTCTTTAAATT |
| | QSTSDGSKSLQELFSV | | CGGCCTCGATCTCATCAGAAAGGCTCCGGTATCTTCAAAACAATCA |
| | PDDKLDGLKFIKEACE | | ACTTCAGATGGCTCGAAGTCTCTTCAAGAGCTATTTTCTGTGCCGG |
| | ALPEASLLCGEKGELL | | ACGATAAATTAGATGGGCTAAAGTTTATTAAGGAGGCTTGTGAAGC |
| | GYQDFRTSFAGHIDSW | | CCTTCCAGAAGCTTCTTTGCTTTGTGGAGAAAAAGGAGAACTGCTG |
| | VANYVNRLFELIELVN | | GGGTATCAAGACTTCAGAACTTCTTTTGCCGGTCATATAGACAGTT |
| | QLPESIKLPSILTQKN | | GGGTTGCTAACTACGTAAATCGCCTTTTTGAGCTCATAGAATTAGT |
| | HNLVASLGLQEAEVSH | | AAATCAATTACCGGAGTCAATTAAGCTTCCTTCAATACTTACGCAG |
| | SLELFEGLVKNVRQTL | | AAGAATCATAATCTTGTTGCATCCCTGGGACTTCAGGAAGCAGAAG |
| | KKLAGIDISSSPNEQD | | TTAGCCATTCGTTGGAGCTGTTTGAAGGTCTTGTTAAAAATGTTCG |
| | IKEFYAFSDVLNRLGS | | TCAAACTTTAAAAAAATTGGCAGGCATAGACATCTCGAGTTCTCCG |
| | IRNQIENAVQTAKKDK | | AATGAACAAGATATAAAAGAGTTCTATGCATTTTCAGATGTGCTTA |
| | IDLESAIEWKEWKKLK | | ATCGGCTGGGTTCAATCCGAAATCAAATTGAAAATGCTGTTCAAAC |
| | KLPKLNGLGGGVPKQQ | | AGCCAAGAAGGATAAAATTGACCTTGAGTCGGCAATAGAGTGGAAA |
| | ELLDKALESVKQIRHY | | GAGTGGAAAAAGTTAAAAAAACTCCCGAAATTAAATGGTCTTGGCG |
| | QRIDFERVIQWAVNEH | | GAGGAGTTCCAAAACAACAGGAATTACTTGATAAAGCGCTGGAGAG |
| | CLETVPKFLVDAEKKK | | CGTAAAACAGATAAGACATTACCAACGGATCGATTTCGAGAGAGTA |
| | INKESSTDFAAKENAV | | ATCCAGTGGGCGGTAAACGAACATTGCCTGGAAACGGTTCCTAAAT |
| | RFLLEGIGAAARGKTD | | TTTTGGTTGATGCCGAAAAAAAGAAGATTAATAAAGAATCTTCTAC |
| | SVSKAAYNWFVVNNFL | | GGATTTTGCTGCAAAAGAAAATGCGGTTCGCTTCCTACTTGAGGGG |
| | AKKDLNRYFINCQGCI | | ATCGGAGCAGCTGCTAGGGGAAAAACTGACTCTGTAAGCAAAGCTG |
| | YKPPYSKRRSLAFALR | | CTTACAATTGGTTTGTTGTAAATAATTTCCTCGCCAAGAAAGACCT |
| | SDNKDTIEVVWEKFET | | GAATCGCTATTTCATTAATTGTCAAGGATGTATTTATAAACCACCT |
| | FYKEISKEIEKFNIFS | | TACTCGAAACGTAGGAGTTTGGCGTTTGCTTTAAGAAGCGACAATA |
| | QEFQTFLHLENLRMKL | | AAGACACGATTGAAGTTGTTTGGGAAAAATTTGAGACTTTCTATAA |
| | LLRRIQKPIPAEIAFF | | AGAAATTTCAAAAGAAATAGAGAAATTCAATATTTTTAGCCAGGAA |
| | SLPQEYYDSLPPNVAF | | TTCCAAACATTTCTTCACTTAGAAAATCTGCGTATGAAACTTCTTT |
| | LALNQEITPSEYITQF | | TGAGAAGAATACAAAAGCCGATACCAGCGGAGATAGCGTTTTTTTC |
| | NLYSSFLNGNLILLRR | | TCTTCCACAGGAGTACTATGACTCCCTTCCTCCTAACGTAGCATTT |
| | SRSYLRAKFSWVGNSK | | CTTGCTTTGAATCAGGAAATTACACCCTCGGAGTATATAACCCAAT |
| | LIYAAKEARLWKIPNA | | TTAATCTATATTCGAGTTTCCTGAACGGTAATCTTATTCTGCTGCG |
| | YWKSDEWKMILDSNVL | | AAGAAGCAGGTCTTATCTGCGAGCTAAATTTAGTTGGGTGGGTAAT |
| | VFDKAGNVLPAPTLKK | | AGTAAACTTATCTATGCGGCAAAAGAAGCTAGATTATGGAAAATTC |
| | VCEREGDLRLFYPLLR | | CCAATGCATATTGGAAATCGGACGAATGGAAGATGATCCTAGATTC |
| | QLPHDWCYRNPFVKSV | | TAATGTGCTGGTGTTTGATAAAGCCGGCAATGTTCTTCCGGCACCG |
| | GREKNVIEVNKEGEPK | | ACCCTGAAAAAGGTGTGTGAACGTGAAGGTGATCTGAGGCTTTTCT |
| | VASALPGSLFRLIGPA | | ACCCACTTTTAAGACAACTCCCCCATGATTGGTGCTACAGAAATCC |
| | PFKSLLDDCFFNPLDK | | CTTTGTGAAGAGCGTGGGCAGAGAAAAAAACGTCATTGAAGTTAAC |
| | DLRECMLIVDQEISQK | | AAAGAAGGGGAACCTAAAGTTGCTTCGGCTTTACCGGGATCTTTGT |
| | VEAQKVEASLESCTYS | | TCCGACTGATAGGTCCTGCACCTTTTAAATCTCTGCTGGATGATTG |
| | IAVPIRYHLEEPKVSN | | TTTCTTTAATCCTTTAGATAAGGACCTACGGGAATGCATGCTTATT |
| | QFENVLAIDQGEAGLA | | GTCGATCAGGAAATAAGCCAGAAGGTAGAAGCTCAGAAAGTGGAGG |
| | YAVFSLKSIGEAETKP | | CCTCATTGGAATCATGTACTTATTCAATCGCAGTCCCGATTAGATA |
| | IAVGTIRIPSIRRLIH | | CCACTTAGAGGAACCGAAGGTATCCAATCAATTTGAAAATGTTTTG |
| | SVSTYRKKKQRLQNFK | | GCTATTGATCAGGGAGAAGCCGGACTTGCATATGCGGTGTTCTCTC |
| | QNYDSTAFIMRENVTG | | TTAAGAGTATTGGTGAAGCGGAAACCAAGCCAATTGCGGTCGGTAC |
| | DVCAKIVGLMKEFNAF | | AATCAGAATTCCGTCCATTAGGCGATTGATTCATTCTGTCTCGACT |
| | PVLEYDVKNLESGSRQ | | TACAGAAAAAAGAAGCAGCGACTGCAAAACTTTAAGCAAAATTACG |
| | LSAVYKAVNSHFLYFK | | ATTCAACAGCCTTCATTATGCGTGAGAACGTGACAGGAGATGTATG |
| | EPGRDALRKQLWYGGD | | CGCAAAGATTGTTGGTTTAATGAAGGAATTCAATGCGTTTCCAGTT |
| | SWTIDGIEIVTRERKE | | CTGGAATATGACGTTAAAAATTTAGAGTCCGGAAGCAGACAGCTGT |
| | DGKEGVEKIVPLKVFP | | CAGCAGTCTATAAGGCTGTGAACTCCCACTTCTTGTACTTTAAGGA |
| | GRSVSARFTSKTCSCC | | GCCTGGCAGAGATGCATTAAGAAAGCAGCTTTGGTACGGCGGAGAT |
| | GRNVFDWLFTEKKAKT | | TCTTGGACTATTGACGGAATTGAGATTGTGACAAGGGAGAGGAAAG |
| | NKKFNVNSKGELTTAD | | AGGACGGAAAAGAAGGGGTGGAAAAGATTGTTCCGCTGAAAGTTTT |
| | GVIQLFEADRSKGPKF | | CCCCGGGCGTTCCGTTTCTGCTCGATTCACGAGCAAGACCTGTTCC |
| | YARRKERTPLTKPIAK | | TGCTGCGGGAGGAATGTTTTTGATTGGCTCTTCACTGAAAAGAAAG |
| | GSYSLEEIERRVRTNL | | CGAAGACAAATAAGAAGTTTAATGTAAACAGTAAAGGAGAACTTAC |
| | RRAPKSKQSRDTSQSQ | | GACTGCTGACGGCGTCATTCAGCTGTTTGAAGCAGATCGATCCAAA |
| | YFCVYKDCALHFSGMQ | | GGTCCTAAGTTTTATGCGAGAAGAAAGGAGAGGACGCCCCTAACAA |
| | ADENAAINIGRRFLTA | | AGCCAATAGCAAAGGGCTCTTACTCTCTGGAAGAGATTGAAAGGCG |
| | LRKNRRSDFPSNVKIS | | TGTGCGCACAAATCTAAGAAGAGCCCCTAAATCCAAGCAATCGAGA |
| | DRLLDN | | GATACGAGTCAGAGTCAATATTTTTGCGTTTATAAAGATTGTGCGC |
| | | | TTCATTTTTCCGGCATGCAGGCAGACGAAAACGCTGCAATTAATAT |
| | | | TGGGCGCCGGTTCCTTACTGCATTGAGAAAAAATCGCAGGAGTGAT |
| | | | TTTCCTTCGAACGTAAAGATATCTGACAGATTGTTAGATAATTAA |
| 15 | MKTENRGLMYSAPFIK | 47 | ATGAAAACAGAGAATAGGGGGCTGATGTACTCAGCCCCCTTTATTA |
| | LVEGNLMTSNETTTQS | | AACTAGTGGAAGGGAACTTAATGACTTCAAACGAAACAACTACACA |
| | KDFKNYRHPRFNPKKD | | AAGTAAAGATTTCAAGAACTACAGACATCCCAGATTTAATCCTAAA |
| | GSLKKVIQIVKDGVPT | | AAAGACGGATCTTTAAAAAAAGTAATACAAATCGTTAAAGATGGTG |
| | YGDFNPHEQKKDKTPN | | TGCCTACGTATGGGGACTTCAACCCTCATGAACAAAAGAAGGATAA |
| | RRGHLLSRGESRKLKR | | AACCCCTAATAGGAGAGGACATCTTCTTAGTAGGGGAGAAAGTCGA |
| | SIRALSDSQKKGEGAF | | AAATTAAAACGAAGTATAAGGGCTCTCAGCGATTCTCAAAAAAAAG |
| | EQTRTIVCKTKGDVRP | | GAGAAGGGGCCTTTGAGCAAACTCGAACAATAGTATGTAAAACTAA |
| | EANFELMRETYNTLNA | | GGGAGATGTCCGACCAGAAGCTAATTTCGAGTTAATGCGGGAGACT |
| | LDFGSLKFSFLALPFL | | TATAACACTTTGAACGCACTTGATTTCGGATCTCTAAAATTCTCTT |
| | GMIASPKTAQCVINEN | | TCTTAGCTCTTCCTTTTCTAGGAATGATAGCGTCCCCTAAAACCGC |
| | REHFVPDGLFESIVKD | | CCAATGTGTGATAAATGAAAATCGTGAGCATTTTGTTCCCGATGGA |
| | LGVLGEGMTPETIALA | | TTATTCGAGAGTATCGTAAAGGACCTGGGAGTTCTAGGGGAGGGTA |
| | VQAEPGKKCCPDKGRV | | TGACCCCGGAGACCATAGCATTGGCTGTCCAGGCTGAACCTGGGAA |
| | EFKKVLFRAFPKSMGF | | AAAATGTTGCCCGGATAAAGGAAGGGTAGAGTTTAAAAAAGTCCTG |
| | SLEYVINQIKGLTAEL | | TTTCGGGCATTCCCTAAATCAATGGGATTTTCTTTAGAGTACGTCA |
| | PKLESEVAALKSEVAQ | | TCAATCAAATAAAAGGCTTAACCGCTGAACTACCAAAATTAGAGTC |
| | IEKDLQELSEESKKYQ | | CGAAGTAGCTGCTCTAAAGTCTGAGGTCGCTCAGATTGAAAAAGAC |
| | RTEKSLLKKESDLKTK | | TTACAAGAGTTATCAGAAGAGTCTAAAAAGTACCAAAGGACTGAGA |
| | ELKLAEYEQKLTNYKA | | AGAGCTTATTGAAAAAAGAATCGGATTTAAAAACAAAGGAATTAAA |
| | ERDSFFKVDDFIQEVL | | GCTTGCTGAATATGAGCAAAAGTTAACAAATTATAAAGCAGAACGG |
| | DNVVACSEVKDRADFL | | GACTCTTTCTTTAAAGTCGATGACTTCATTCAAGAAGTTTTAGATA |
| | NLDKKVYVYECFDLAL | | ATGTGGTTGCCTGTAGTGAAGTCAAAGATAGAGCCGATTTTCTGAA |
| | KKLNPEYSGRLTSLAS | | CTTAGACAAAAAAGTCTACGTTTATGAGTGTTTTGATCTTGCTCTG |
| | FFKNHKPKGRTIAFVP | | AAGAAACTTAATCCAGAATACTCGGGTCGATTAACTTCACTAGCCT |
| | DLEYKGMDFLKDNADI | | CATTTTTCAAAAATCACAAGCCTAAGGGAAGAACAATAGCTTTCGT |
| | LPYLNFSALMNRLVSL | | TCCTGATCTTGAGTACAAAGGGATGGACTTCCTAAAAGATAATGCT |
| | GLLKRGEFSPKVITTF | | GATATTCTTCCTTACTTAAATTTCTCAGCCCTCATGAATCGGTTAG |
| | NDLVLSPNNDALNSFL | | TTAGCCTCGGTCTTTTAAAAAGAGGCGAGTTTAGTCCTAAGGTGAT |
| | GVGFEKIRTSSLEELR | | TACTACCTTTAATGATCTCGTCCTATCCCCAAACAATGACGCTCTT |
| | DYFNVESDKDDTIKAL | | AATAGTTTTTTAGGAGTGGGGTTTGAAAAAATAAGAACTTCCAGTC |
| | QELLTLAVEDHVFGKK | | TGGAAGAGCTTAGGGATTACTTTAACGTAGAATCCGACAAAGACGA |
| | SYSEFRVEVGSQIKSF | | CACTATAAAAGCTCTTCAAGAACTACTCACCCTGGCTGTAGAAGAC |
| | YSNHGARCIAFYNASI | | CATGTCTTTGGTAAAAAAAGCTATTCTGAATTTAGAGTAGAAGTCG |
| | DPSPIEVSGDLWNLSN | | GTTCTCAAATTAAATCCTTTTACAGCAATCACGGAGCTCGATGTAT |
| | AYLFRKTYANPEFLKN | | TGCGTTTTATAACGCTTCAATCGATCCTTCTCCAATCGAAGTCTCT |
| | QIESVNSSGEVLKSRL | | GGGGATTTATGGAATCTTTCGAATGCCTATTTATTTCGAAAGACTT |
| | FRIMGYSEELPSREDI | | ATGCCAACCCTGAATTTCTTAAAAATCAAATCGAGAGCGTGAATTC |
| | EFFKDFSRLVDRTFSD | | TTCTGGAGAAGTTCTTAAAAGTAGATTATTCCGTATAATGGGTTAT |
| | IERINTSLKEEIKTYD | | TCCGAAGAACTTCCTTCTAGAGAAGATATTGAGTTCTTTAAGGATT |
| | PKTDKKIIKSLEDLII | | TCAGCCGTTTAGTAGACCGAACTTTTTCAGATATAGAACGCATCAA |
| | ITPKWAKDIKKLAGVG | | CACCTCTTTAAAAGAGGAAATAAAGACTTATGATCCAAAGACAGAT |
| | GGKKSAKEEAQDLIDR | | AAAAAAATAATCAAGAGTCTAGAGGACCTCATAATCATAACCCCGA |
| | FNLIRSRLNTQVDAVV | | AATGGGCGAAAGATATTAAAAAGCTAGCTGGAGTAGGCGGAGGTAA |
| | VTAGNLETMEVHKQVS | | GAAGTCTGCAAAAGAAGAGGCTCAGGATCTGATTGATCGTTTCAAC |
| | LDALKSNLDYDSTVDY | | TTAATACGCTCAAGACTCAATACTCAAGTGGACGCTGTAGTAGTTA |
| | DELYYRETFDSLIRLI | | CAGCAGGCAATCTTGAGACTATGGAAGTACATAAACAAGTTTCTCT |
| | RDTNCPPLIKKFRDDV | | AGACGCTCTTAAATCAAACCTGGATTACGATTCAACCGTGGATTAC |
| | VSYGLVRGSGIKPLTV | | GACGAGCTCTACTACAGAGAAACCTTCGACTCCTTAATAAGATTGA |
| | YINSGKGRVFVHPKSN | | TTCGAGATACTAATTGCCCTCCATTGATTAAAAAATTTAGGGACGA |
| | YKHSALNIELSILEKF | | TGTGGTATCTTACGGCCTAGTTCGAGGTAGTGGCATAAAGCCGCTG |
| | NPVAYLEGLLEYLDSI | | ACCGTGTACATCAACTCTGGGAAAGGAAGAGTGTTTGTCCACCCTA |
| | PRLKPGRLFCLKETIR | | AAAGTAATTACAAACATAGTGCTTTGAACATCGAACTGAGTATCCT |
| | LEIIKFKIFNIPDTVP | | TGAAAAGTTCAATCCTGTTGCTTACCTTGAAGGCCTTTTAGAGTAC |
| | VSSINQDYFDLIEGRT | | TTAGATTCAATCCCCAGGTTAAAACCAGGACGGCTGTTTTGTTTGA |
| | FLSDITRDKDEILNSE | | AAGAAACTATTAGGTTGGAGATCATTAAATTTAAGATCTTCAATAT |
| | FRTIINCYITTLRSII | | TCCAGACACCGTTCCCGTATCTTCGATTAATCAAGATTATTTTGAT |
| | PDVTQEGVSLRLTFRK | | TTGATCGAGGGTAGAACTTTTTTAAGCGATATCACGAGAGATAAAG |
| | KGTSTVMGVPKHEEVT | | ATGAGATATTGAACTCCGAGTTCAGGACGATCATAAACTGCTACAT |
| | TNEKGETESIFKFTLP | | AACAACTTTGAGATCTATCATACCTGACGTTACCCAAGAAGGGGTG |
| | ASLEYSKGPLSTLISE | | TCCTTGAGGCTTACTTTTAGGAAAAAAGGAACCAGTACCGTGATGG |
| | LKNPSSVFKVESKVTK | | GAGTTCCTAAACACGAAGAAGTCACTACTAATGAGAAAGGTGAAAC |
| | ESFPKIEVSVGKGHQV | | GGAGTCTATCTTCAAGTTCACTTTACCTGCAAGTTTAGAGTATTCT |
| | VKLSERSIKKLTSPEL | | AAGGGTCCTTTAAGCACTCTGATCTCAGAATTAAAGAATCCCTCAT |
| | VASDGKKIPNPYLEGV | | CAGTGTTTAAAGTCGAAAGTAAAGTAACCAAAGAGAGTTTCCCTAA |
| | SQLLNYIPQNLCIETG | | AATCGAGGTCTCGGTGGGTAAGGGCCATCAAGTTGTAAAACTATCG |
| | WKVSKSDKDTRGITFG | | GAGCGAAGTATTAAAAAACTAACTAGCCCGGAGTTAGTCGCAAGCG |
| | KKKLGFKSAPGVLKLV | | ACGGTAAAAAGATCCCCAATCCATACTTAGAAGGAGTTTCACAACT |
| | SGATQLQTVQQSLIDE | | TCTAAACTACATACCGCAAAATCTTTGCATTGAAACCGGATGGAAG |
| | DINLGDVEYVFEQKYK | | GTTTCAAAAAGCGATAAGGACACAAGAGGGATTACTTTCGGCAAAA |
| | KKLDFRGDQVFLRNVK | | AGAAACTGGGTTTCAAGTCTGCTCCAGGTGTTCTTAAACTGGTGTC |
| | IEHSKNKPEVEVFLNI | | CGGAGCCACGCAACTGCAGACGGTGCAACAATCTTTAATAGATGAG |
| | PVTEKRLIKKSAISPF | | GACATTAACCTAGGCGATGTGGAGTATGTTTTTGAGCAGAAGTATA |
| | NHTADIGFDLGEYGLA | | AAAAGAAGCTTGATTTTAGAGGAGATCAGGTTTTTTTAAGAAATGT |
| | YAVLDIRTGEIKATGF | | GAAGATTGAACATTCTAAGAATAAACCCGAGGTCGAAGTTTTTCTC |
| | VPIKMFRKLINVVNSY | | AACATCCCGGTTACTGAGAAGAGGCTTATTAAGAAATCCGCGATCT |
| | RKHNQPRRDYSKFSDS | | CTCCTTTTAACCACACCGCCGATATTGGATTTGATCTGGGGGAGTA |
| | KLQNMKEAATAEICTI | | TGGTCTGGCTTATGCGGTTTTGGATATTAGAACAGGTGAAATCAAA |
| | IWSLMDLHNALPVFEN | | GCTACTGGATTCGTTCCAATCAAGATGTTTCGAAAACTGATAAATG |
| | NVSGLSRGKNAVRNIY | | TCGTTAACTCGTATCGAAAGCACAACCAGCCCCGGAGAGACTACAG |
| | ANVVDYFVRNSSNAAS | | TAAATTCAGTGATTCTAAACTTCAGAATATGAAAGAAGCAGCTACC |
| | QSRLKHSFYGDIKITR | | GCTGAGATTTGTACGATTATTTGGAGTTTGATGGATCTTCATAACG |
| | TDGKGKKIFYSPGRVV | | CTCTTCCCGTATTTGAGAATAATGTTTCAGGATTGTCCAGAGGTAA |
| | SGAYTSQECSCCGKNP | | GAACGCAGTTCGGAATATTTACGCTAACGTAGTAGATTACTTCGTC |
| | VRMVRYSDVEEYSIDS | | AGAAATTCGAGCAACGCGGCATCCCAGTCTAGACTCAAGCATTCTT |
| | EGAVILDGEFKYYLKA | | TCTATGGAGACATTAAGATTACTCGGACAGATGGAAAGGGAAAGAA |
| | TAKNNTSKHTRADFTA | | GATATTTTATTCCCCAGGAAGAGTTGTTTCTGGAGCCTACACAAGC |
| | AFKPGGKIRKKDLISR | | CAAGAATGTAGTTGTTGTGGTAAGAATCCTGTTCGAATGGTTAGAT |
| | IKLQMRRAPVDKRTKN | | ACTCTGATGTTGAGGAGTACTCCATCGATTCCGAGGGGGCGGTCAT |
| | SSQSRYVCLFDDCSLV | | TCTAGACGGAGAGTTTAAGTATTATTTGAAGGCAACTGCTAAAAAT |
| | EMSADTNAAINIVKR | | AACACTTCAAAGCATACCCGTGCAGACTTCACCGCAGCGTTTAAAC |
| | | | CAGGAGGCAAGATCCGCAAAAAAGACCTCATCTCTAGGATTAAATT |
| | | | GCAAATGCGAAGGGCTCCCGTAGATAAAAGAACGAAGAACTCCTCC |
| | | | CAGTCGAGGTATGTCTGCTTGTTTGATGATTGTTCATTGGTCGAAA |
| | | | TGAGTGCTGACACTAATGCAGCAATAAATATTGTAAAAAGATGA |
| 16 | MIKTFKTAVFNVKFSK | 48 | ATGATCAAAACATTTAAGACCGCGGTTTTCAATGTTAAGTTTAGTA |
| | RKGNIIDTQMRLAENA | | AGAGAAAAGGAAATATAATAGATACACAGATGCGTTTAGCCGAAAA |
| | FYDVIERLAHHVEPLI | | CGCATTCTATGATGTTATAGAACGGCTGGCACATCATGTTGAGCCC |
| | KLNKEQRKDMLTRLKK | | CTTATTAAGCTAAATAAAGAACAAAGAAAGGACATGTTAACACGGC |
| | EASQLIKPHPLSNASK | | TTAAAAAAGAGGCTAGTCAACTTATAAAACCACATCCTTTATCCAA |
| | SGVVADAIAQISSTVE | | CGCCTCTAAATCAGGTGTCGTCGCAGATGCAATCGCACAGATTAGT |
| | LRLTGQDAKLPTRNNR | | TCAACTGTTGAGCTTCGTTTAACGGGGCAAGATGCAAAATTACCCA |
| | DIDTYDIGMDMLVGSL | | CACGAAACAATAGAGACATCGACACTTATGATATTGGAATGGATAT |
| | DLESQDLAKQLIYSKP | | GTTAGTTGGTTCGTTAGATCTTGAATCTCAAGATTTGGCTAAGCAA |
| | YDGMPRPLLWLRTRPS | | TTAATATATTCAAAGCCTTATGATGGTATGCCAAGACCTTTACTAT |
| | DGAMLLRDGLGRYFVY | | GGCTTAGGACTCGGCCATCGGATGGGGCTATGTTGTTGAGAGATGG |
| | INSHSSKSKFSKAKVV | | TTTAGGCAGATATTTTGTTTATATTAACAGTCATAGTTCTAAGTCG |
| | INDLVNVRTGETENFS | | AAGTTTTCGAAAGCAAAAGTTGTTATTAATGATTTGGTGAATGTTC |
| | SSTGLLLPIQLSKWHQ | | GAACAGGGGAAACCGAAAACTTCTCTAGTTCCACTGGTTTATTGTT |
| | SEFLAKGKPKSYRLIK | | GCCCATACAACTCTCTAAGTGGCATCAATCTGAATTTTTGGCTAAA |
| | KADGYILAVTFEFKAE | | GGTAAACCTAAATCTTATCGATTAATAAAAAAAGCAGACGGGTACA |
| | KIEPATYLGVDRGIDK | | TATTGGCAGTCACTTTTGAATTTAAAGCTGAAAAAATTGAGCCTGC |
| | IAAFAVTSKKEVLKKD | | TACCTATTTGGGGGTAGATCGCGGTATAGACAAAATTGCGGCTTTT |
| | FCDGNELRDYQKECET | | GCTGTTACTTCAAAAAAAGAAGTTTTAAAGAAAGATTTTTGTGATG |
| | NARKKQTKGNAKYIRW | | GCAATGAACTAAGAGACTATCAAAAAGAATGTGAGACAAACGCTAG |
| | RGYTDLIMHKIANEIV | | AAAAAAACAAACAAAAGGCAATGCCAAGTATATCCGATGGCGTGGT |
| | NTALKYRSQVVLEDLT | | TAGACTGATTTAATAATGCATAAAATTGCAAATGAGATCGTCAATA |
| | NIANGHHHRRARFARK | | CAGCGCTAAAGTATAGGTCACAAGTAGTTTTGGAAGATTTGACAAA |
| | TNFNKVLSRQQYQKLQ | | TATTGCGAACGGACACCATCACAGGCGAGCTAGGTTTGCACGAAAA |
| | HLLNYKLSYVGLPTPL | | ACTAATTTCAATAAAGTTTTAAGTCGTCAGCAATATCAAAAATTAG |
| | FVRAAGTSITCNRCGN | | AGCATTTATTAAATTATAAGCTGTCCTACGTTGGGCTACCAACACC |
| | YDSKNRDLNERSLFLC | | GCTATTTGTTAGAGCTGCAGGTACATCAATTACTTGCAATAGGTGT |
| | KSCNYQDNADVNAAVT | | GGGAACTATGATTCTAAAAATCGCGACCTAAATGAGCGATCGTTGT |
| | ISMKGEWLTTQFDKEH | | TTTTGTGTAAAAGCTGTAATTATCAAGATAACGCTGATGTTAATGC |
| | KKMKNRFSDWIPLPS | | AGCAGTTACAATTTCAATGAAAGGTGAATGGTTGACTACTCAGTTC |
| | | | GATAAAGAACATAAAAAAATGAAAAATAGGTTCTCAGACTGGATCC |
| | | | CTTTACCATCATAG |
| 17 | MSDVVTSFLTVKYKLH | 49 | ATGTCAGATGTCGTCACATCGTTTCTGACGGTGAAGTACAAACTTC |
| | NPSKRRRAMLLDAMRR | | ACAATCCATCCAAACGCCGCAGAGCGATGTTGCTGGATGCGATGCG |
| | AHLGYDKLLKRVREDV | | TCGCGCCCATTTGGGATACGACAAGCTGCTGAAGCGGGTTCGTGAA |
| | EAIVDITERQERTDAE | | GATGTTGAGGCGATTGTCGACATCACTGAACGCCAGGAACGCACGG |
| | KELTKKLQALAKPLPL | | ACGCCGAGAAGGAGCTAACCAAGAAACTCCAGGCGCTTGCCAAGCC |
| | GNGPKQAIIADALAQS | | CCTCCCCCTTGGGAATGGCCCAAAACAGGCGATCATCGCAGATGCA |
| | KSYVELKKADPNTSYP | | TTGGCCCAATCTAAAAGTTATGTCGAGTTGAAGAAGGCCGACCCAA |
| | TTPRLKVDQVDYDAAV | | ATACCTCATACCCAACCACACCTAGGTTGAAAGTCGACCAAGTAGA |
| | DGIANSQSILEENEYR | | CTATGACGCAGCGGTGGATGGGATAGCAAACTCGCAGTCCATCCTT |
| | DLLAKLSRPGLPRPLN | | GAAGAAAATGAATATCGAGACCTACTGGCAAAACTGTCGCGTCCTG |
| | ILKNRIGDGALLLQDD | | GCCTGCCTCGTCCACTGAACATCCTGAAGAACCGGATCGGGGATGG |
| | NGRLFVFINLLPKTAK | | CGCGCTGCTTTTGCAAGACGACAATGGTCGCCTGTTCGTTTTCATC |
| | RKRKVDLTGLIDTRTG | | AACCTGCTGCCGAAGACGGCTAAACGCAAACGAAAGGTCGACCTGA |
| | EIMQKSTSSGDIFPLE | | CGGGTCTGATCGATACCCGTACAGGCGAGATTATGCAGAAGTCGAC |
| | CGKWHDEKFLKQGTLQ | | ATCAAGCGGCGATATTTTCCCCTTGGAATGTGGGAAGTGGCATGAC |
| | SSRLIYDGKDFYFAAT | | GAGAAGTTCTTAAAACAAGGAACGCTCCAGTCCAGTCGGCTGATAT |
| | FQFEAPLREPTNYIGV | | ATGACGGTAAAGACTTCTACTTCGCCGCGACGTTCCAGTTTGAGGC |
| | DRGIELLAAWSVIDDK | | ACCTCTACGGGAGCCGACGAATTACATTGGTGTAGATCGTGGCATT |
| | GRKLDAGYHGGERLRS | | GAGCTGCTGGCAGCTTGGTCAGTGATCGACGACAAGGGCCGCAAGC |
| | FQRRQEQDQKDTQRRG | | TAGATGCGGGATACCATGGCGGCGAACGCCTCAGGAGTTTTCAACG |
| | KIYTSRTRRAVADEEV | | CAGACAAGAGCAAGATCAGAAAGACACGCAGCGTCGGGGAAAGATT |
| | HIVANKIVDMAAKHNA | | TACACCAGCCGAACCCGCCGTGCAGTCGCCGATGAGGAAGTCCACA |
| | VVVLEDLKTITMGPHQ | | TCGTCGCAAATAAGATCGTGGACATGGCGGCAAAACATAACGCTGT |
| | KRPKGARKSGFRRMLT | | CGTCGTCCTGGAAGACCTGAAGACCATTACGATGGGGCCACATCAG |
| | RAQYAKLKHCVDYRLK | | AAACGGCCCAAGGGAGCCAGGAAGAGTGGCTTCCGACGAATGCTAA |
| | MEGFAPLRRNSPSYME | | CCCGCGCCCAATATGCCAAGCTGAAGCACTGCGTCGACTACCGGCT |
| | IHPAYTSLTCAKCAHQ | | AAAGATGGAAGGGTTCGCGCCTCTACGTCGTAACAGCCCCAGCTAT |
| | DKESRQSQAVFVCTKC | | ATGGAAATTCATCCGGCCTACACCAGTCTGACCTGCGCCAAGTGTG |
| | GHKDNADENAAVNVAA | | CGCACCAAGACAAGGAAAGCAGACAATCACAGGCAGTCTTCGTCTG |
| | KGIHFDQIVKGRKKGQ | | CACCAAGTGTGGACACAAAGACAACGCCGATGAAAACGCTGCGGTG |
| | KLKDHEQFSAWYADLK | | AATGTCGCGGCAAAAGGCATCCACTTCGATCAGATCGTGAAAGGGA |
| | NGGGGHADGP | | GGAAGAAGGGACAGAAACTCAAAGACCATGAACAGTTTTCGGCGTG |
| | | | GTACGCCGATCTGAAAAACGGGGGTGGAGGCCATGCAGATGGTCCT |
| | | | TAG |
| 18 | MKATTITKSLRFWIDE | 50 | TTGAAGGCAACGACCATCACAAAGAGCCTCCGCTTCTGGATCGACG |
| | PPERCRLLYGVSDELT | | AACCGCCTGAGCGGTGCCGTCTCCTCTATGGCGTCAGCGATGAACT |
| | DAYNAILEYWETDVRK | | CACCGATGCATACAACGCTATCCTCGAGTACTGGGAGACCGATGTC |
| | VAVEATEAAIEAYHVW | | CGCAAGGTAGCCGTTGAGGCCACCGAGGCGGCAATCGAGGCATACC |
| | KDAPKETRGDKPKWWS | | ACGTATGGAAGGACGCTCCCAAGGAGACGCGCGGAGACAAGCCCAA |
| | ADTATRAAFLRVGVAA | | GTGGTGGTCAGCGGACACGGCAACCAGAGCGGCGTTCCTGCGAGTC |
| | TSTIRTRLVDNMVQGE | | GGAGTCGCCGCAACATCGACCATCCGCACTCGGCTAGTAGACAACA |
| | WLRDVKTYMSRRFKDH | | TGGTGCAGGGCGAGTGGCTGCGGGACGTGAAGACCTACATGAGCCG |
| | IAAGIRHRAKGVVISL | | CCGGTTCAAGGACCACATCGCCGCCGGCATCCGGCACCGCGCGAAG |
| | DQSTMKPDVQLGDDGR | | GGCGTTGTCATCTCTCTCGACCAGAGCACAATGAAGCCGGACGTGC |
| | WTIRCALWAQGTEESA | | AGCTCGGTGACGATGGGCGCTGGACGATCCGGTGCGCGCTATGGGC |
| | RNNDRWLLSPYDRKRQ | | GCAGGGCACCGAGGAGTCGGCACGCAACAACGACCGATGGCTCCTC |
| | TWTIANLLEADQHGEV | | TCGCCATATGACCGGAAGCGGCAGACGTGGACGATCGCGAACCTGC |
| | RGVKLVPPKPGAPAGK | | TCGAAGCCGACCAGCACGGCGAGGTCCGGGGCGTCAAGCTAGTCCC |
| | RRWSAMITVTLPVESS | | GCCGAAGCCGGGCGCACCGGCAGGCAAGCGCCGATGGTCGGCCATG |
| | VLDEERPNVAGVDMGL | | ATCACCGTGACGCTGCCCGTGGAGTCCAGCGTGCTAGACGAGGAGC |
| | THFAVYSCPARNTFEF | | GCCCGAACGTGGCGGGCGTGGACATGGGCCTCACGCACTTCGCGGT |
| | VSSRELQAAMEKARRR | | GTATAGCTGCCCGGCCAGGAACACATTCGAGTTCGTCTCATCGAGA |
| | RRGVPRKRSTALGQKL | | GAGTTGCAGGCAGCGATGGAGAAGGCGCGCCGCAGGCGGCGGGGTG |
| | GRRQDALCRLTARRLI | | TCCCTCGCAAGCGGAGTACGGCACTCGGCCAGAAGTTGGGCAGGCG |
| | DCCRRDRVGTLRVEDL | | TCAGGATGCGCTCTGCCGACTGACCGCTAGGCGGCTCATTGATTGC |
| | TGIRDQGSDDADRNFA | | TGCCGGCGTGATCGGGTCGGGACCCTGCGCGTGGAAGACCTGACAG |
| | LGARFPYYKLQTYLEQ | | GCATCCGCGACCAGGGCAGCGATGATGCCGACCGGAACTTCGCGCT |
| | AAASAGVRLEKVQPAG | | CGGTGCGCGGTTCCCGTACTACAAGCTACAGACGTATCTAGAGCAA |
| | TSQTCSRCAVRDPESR | | GCCGCTGCGTCGGCCGGGGTGCGGCTGGAGAAGGTTCAGCCTGCCG |
| | DGKRFVCRHCGYKGDA | | GCACGAGTCAGACGTGTTCGCGGTGCGCGGTGCGCGATCCCGAGTC |
| | DLNAANNIASGRFRRS | | TCGGGACGGCAAGCGGTTCGTTTGCCGGCACTGCGGTTACAAAGGC |
| | IRPSTQPAGRIPSAAP | | GATGCCGACCTGAACGCTGCGAACAACATAGCGTCAGGTCGCTTCC |
| | DCTGEAVKASDVDAEL | | GGCGGTCGATCCGACCGTCTACACAGCCGGCGGGACGTATCCCGTC |
| | QPARTPAACGGDPYAG | | GGCCGCGCCCGACTGCACGGGCGAGGCAGTGAAGGCGTCAGACGTT |
| | E | | GACGCTGAGCTGCAACCTGCGCGCACGCCGGCAGCTTGTGGAGGTG |
| | | | ATCCATATGCCGGCGAGTGA |
| 19 | MLKAHVIRLNPTEEQA | 51 | ATGTTAAAAGCGCATGTGATCCGTCTCAACCCAACCGAAGAACAGG |
| | SYFWRCAGIARFTWNW | | CCAGCTACTTCTGGCGCTGCGCGGGGATTGCTCGCTTCACCTGGAA |
| | ALAELNAAYDRGERPA | | TTGGGCGCTGGCTGAGTTGAACGCCGCCTATGACCGGGGTGAACGG |
| | IGSLKLAFNRLRKEEG | | CCTGCGATTGGAAGTCTAAAGCTAGCGTTCAACCGGCTGCGCAAGG |
| | FAPFVGEVQSYAYQQA | | AGGAGGGTTTTGCGCCGTTCGTGGGAGAGGTTCAGAGCTATGCCTA |
| | FTDLQKALSRYHDFRK | | CCAGCAGGCGTTCACCGACCTGCAAAAAGCCCTGAGCCGTTACCAC |
| | RGLLKPPAGWKGRKDH | | GACTTCCGTAAGCGCGGTTTGTTGAAGCCTCCGGCCGGTTGGAAGG |
| | KPFGWPRFKARNRSTP | | GGCGCAAGGATCACAAGCCCTTTGGGTGGCCCCGCTTCAAGGCCCG |
| | AFYLANNGGLRLQGHQ | | CAACCGCAGCACGCCCGCCTTCTATCTGGCGAACAACGGCGGGTTG |
| | VTIQRCPGGPVNMAEQ | | CGGTTGCAGGGCCACCAGGTCACGATCCAGCGCTGTCCGGGCGGCC |
| | LRFAGRVMGGRVRYRA | | CGGTCAACATGGCCGAGCAGTTGCGCTTTGCTGGCAGGGTCATGGG |
| | GHWYLTVQVDVPVEPV | | CGGCCGCGTGCGCTATCGGGCCGGGCACTGGTATCTGACCGTCCAG |
| | PAHTGPAVGLDVGIKT | | GTCGATGTACCGGTGGAACCGGTGCCAGCGCACACCGGGCCGGCTG |
| | LAVTSDGEIYDNPKAL | | TCGGGCTGGACGTAGGCATCAAGACGCTGGCAGTCACCAGCGACGG |
| | GRHQRKLRLLQRSLAR | | CGAGATCTACGACAACCCCAAGGCTCTGGGGCGCCACCAGCGCAAG |
| | QTRGGSNYRKTQAKIA | | CTGCGCCTGCTGCAACGGTCTCTGGCCAGACAGACGCGCGGCGGGT |
| | RLHERIANIRKHTLHQ | | CCAACTACCGCAAGACACAGGCCAAGATCGCCCGGCTCCACGAGCG |
| | ISHEITRDYGLIGLED | | GATCGCCAACATCCGCAAGCACACCTTGCACCAGATTAGCCACGAG |
| | LNVAGMLKNGKLARSI | | ATCACCCGTGACTATGGGCTGATTGGGCTGGAAGACTTGAACGTCG |
| | SDVAFGELRRQIGYKS | | CCGGGATGCTCAAGAACGGCAAGTTGGCAAGGTCGATTTCGGACGT |
| | EWRGSRVVIVSRWFPS | | CGCTTTCGGCGAGTTGCGCCGGCAGATCGGGTATAAAAGCGAGTGG |
| | SKTCNECGHVMADMPL | | CGCGGGTCGCGGGTAGTAATTGTTTCCCGTTGGTTCCCATCCAGCA |
| | SVRWWQCPTCGAEHDR | | AGACCTGCAATGAGTGCGGCCACGTCATGGCCGACATGCCGCTATC |
| | DGNAAVNIRNEAVKMA | | GGTGCGCTGGTGGCAGTGCCCGACCTGCGGCGCAGAACATGATCGG |
| | GAA | | GACGGCAACGCAGCGGTCAACATCCGCAACGAGGCCGTGAAGATGG |
| | | | CAGGCGCTGCCTAG |
| 20 | MLKAHVIRLNPTEEQA | 52 | ATGTTAAAAGCGCATGTGATCCGTCTCAACCCAACCGAAGAACAGG |
| | SYFWRCAGIARFTWNW | | CCAGCTACTTCTGGCGCTGCGCGGGGATTGCTCGCTTCACCTGGAA |
| | ALAELNAAYDRGERPA | | TTGGGCGCTGGCTGAGTTGAACGCCGCCTATGACCGGGGTGAACGG |
| | IGSLKLAFNRLRKEEG | | CCTGCGATTGGAAGTCTAAAGCTAGCGTTCAACCGGCTGCGCAAGG |
| | FAPFVGEVQSYAYQQA | | AGGAGGGTTTTGCGCCGTTCGTGGGAGAGGTTCAGAGCTATGCCTA |
| | FTDLQKALSRYHDFRK | | CCAGCAGGCGTTCACCGACCTGCAAAAAGCCCTGAGCCGTTACCAC |
| | RGLLKPPAGWKGRKDH | | GACTTCCGTAAGCGCGGTTTGTTGAAGCCTCCGGCCGGTTGGAAGG |
| | KPFGWPRFKARNRSTP | | GGCGCAAGGATCACAAGCCCTTTGGGTGGCCCCGCTTCAAGGCCCG |
| | AFYLANNGGLRLQGHQ | | CAACCGCAGCACGCCCGCCTTCTATCTGGCGAACAACGGCGGGTTG |
| | VTIQRCPGGPVNMAEQ | | CGGTTGCAGGGCCACCAGGTCACGATCCAGCGCTGTCCGGGCGGCC |
| | LRFAGRVMGGRVRYRA | | CGGTCAACATGGCCGAGCAGTTGCGCTTTGCTGGCAGGGTCATGGG |
| | GHWYLTVQVDVPVEPV | | CGGCCGCGTGCGCTATCGGGCCGGGCACTGGTATCTGACCGTCCAG |
| | PAHTGPAVGLDVGIKA | | GTCGATGTACCGGTGGAACCGGTGCCAGCGCACACCGGGCCGGCTG |
| | LAVTSDGEIYDNPKAL | | TCGGGCTGGACGTAGGCATCAAGGCGCTGGCAGTCACCAGCGACGG |
| | GRHQRKLRLLQRSLAR | | CGAGATCTACGACAACCCCAAGGCTCTGGGGCGCCACCAGCGCAAG |
| | QTRGGSNYRKTQAKIA | | CTGCGCCTGCTGCAACGGTCTCTGGCCAGACAGACGCGCGGCGGGT |
| | RLHERIANIRKHTLHQ | | CCAACTACCGCAAGACACAGGCCAAGATCGCCCGGCTCCACGAGCG |
| | ISHEITRDYGLIGLED | | GATCGCCAACATCCGCAAGCACACCTTGCACCAGATTAGCCACGAG |
| | LNVAGMLENGKLARSI | | ATCACCCGTGACTATGGGCTGATTGGGCTGGAAGACTTGAACGTCG |
| | SDVAFGELRRQIGYKS | | CCGGGATGCTCGAGAACGGTAAGTTGGCAAGGTCGATTTCGGACGT |
| | EWRGSRVVIVSRWFPS | | CGCTTTCGGCGAGTTGCGCCGGCAGATCGGGTATAAAAGCGAGTGG |
| | SKTCNECGHVMADMPL | | CGCGGGTCGCGGGTAGTAATTGTTTCCCGTTGGTTCCCATCCAGCA |
| | SVRWWQCPTCGAEHDR | | AGACCTGCAATGAGTGCGGCCACGTCATGGCCGACATGCCGCTATC |
| | DGNAAVNIRNEAVKMA | | GGTGCGCTGGTGGCAGTGCCCGACCTGCGGCGCAGAACATGATCGG |
| | GAA | | GACGGCAACGCAGCGGTCAACATCCGCAACGAGGCCGTGAAGATGG |
| | | | CAGGCGCTGCCTAG |
| 21 | MLKAHVIRLNPTKEQE | 53 | ATGCTAAAAGCTCACGTGATTCGCCTCAACCCAACCAAAGAACAGG |
| | TYFWRCAGVARFTWNW | | AAACGTATTTCTGGCGCTGCGCGGGGGTTGCCCGCTTCACTTGGAA |
| | ALAELNAAYEKGERPA | | TTGGGCGCTGGCCGAGTTAAACGCAGCTTACGAGAAAGGCGAGCGG |
| | VGSLKLEFNRLRNEEG | | CCTGCCGTTGGAAGTCTTAAGCTGGAGTTCAACCGGCTGCGTAATG |
| | FAPFVGEVQSYAYQQA | | AAGAAGGCTTTGCGCCGTTTGTTGGAGAGGTTCAAAGTTATGCCTA |
| | FGDLQKALSSYHDFRK | | CCAGCAGGCGTTCGGCGACCTGCAAAAAGCCCTGAGCAGCTACCAC |
| | RGMLKPPTSWKGRKDH | | GACTTCCGCAAGCGCGGCATGTTGAAACCTCCTACCAGTTGGAAGG |
| | KPFGWPRFKVRNRSTP | | GACGCAAAGACCACAAGCCATTTGGCTGGCCCCGCTTCAAGGTCCG |
| | AFYLANNGGLRMNGHQ | | CAATCGCAGCACGCCTGCATTCTATCTGGCAAACAATGGCGGATTG |
| | VTIQRCPGGPVNMAEP | | CGAATGAACGGCCATCAGGTGACAATCCAGCGCTGCCCCGGTGGCC |
| | LRFTGKVMGGRVRYRA | | CGGTCAACATGGCGGAACCATTGCGCTTTACCGGCAAGGTCATGGG |
| | GHWYLTVQVDVPVEPD | | CGGCCGCGTGCGCTATCGGGCCGGTCACTGGTATCTGACTGTCCAA |
| | PAHTGPAVGLDVGIKV | | GTCGATGTACCAGTGGAACCGGACCCTGCACACACCGGGCCGGCTG |
| | LAFTSDGVIYDNPKAL | | TTGGGCTGGACGTAGGCATCAAGGTTCTGGCATTTACCAGCGACGG |
| | AHYQRKLRLLQRSLSR | | CGTGATCTACGACAACCCTAAGGCCCTGGCGCACTACCAGCGCAAG |
| | QTRGGSNYRKTQAKIA | | TTACGTTTGTTGCAACGGTCTCTGTCTAGACAGACGCGCGGCGGGT |
| | RLHDRIANIRKHALHQ | | CCAACTATCGCAAGACGCAGGCCAAGATTGCCCGGCTCCATGATCG |
| | VSHEITRDYGLIGLED | | GATCGCCAACATTCGCAAGCACGCCTTGCACCAAGTCAGTCACGAG |
| | LNVSGMLKNGKLARSI | | ATCACGCGCGACTATGGACTGATCGGGCTGGAAGACCTGAACGTTT |
| | SDVALGELRRQIEYKA | | CCGGGATGCTCAAGAATGGCAAACTGGCCCGGTCGATCTCAGACGT |
| | DWRGSRVMIVSRWFPS | | GGCTCTTGGTGAGTTGCGCCGACAGATCGAGTACAAGGCAGACTGG |
| | SKTCNDCGYVMADMLL | | CGCGGGTCGCGGGTAATGATCGTTAGCCGCTGGTTCCCGTCCAGTA |
| | SVRWWQCPACGAEHDR | | AGACATGCAACGACTGCGGCTACGTGATGGCTGACATGCTGCTCTC |
| | DGNAAVNIRNEAVKMA | | GGTGCGCTGGTGGCAGTGCCCGGCCTGCGGTGCAGAACATGATCGG |
| | GAA | | GACGGCAACGCGGCGGTCAACATCCGCAACGAAGCCGTAAAGATGG |
| | | | CAGGCGCTGCCTAG |
| 22 | MYGDSRIEEGNMDALG | 54 | ATGTACGGCGATTCTCGAATCGAGGAAGGAAACATGGACGCGCTCG |
| | KSSRDGTNQHPPCDAA | | GTAAATCGTCCCGAGATGGGACCAATCAGCATCCGCCTTGCGACGC |
| | STAVCETAVIVRATAR | | TGCCTCAACCGCCGTTTGCGAAACGGCCGTCATCGTTCGTGCGACT |
| | MTIDFMAMEEPERIRA | | GCTCGCATGACGATTGACTTCATGGCCATGGAGGAGCCGGAGCGCA |
| | RQLLYEARRNSAAAAN | | TCCGCGCCCGGCAGCTTCTTTACGAGGCACGCAGGAACTCGGCTGC |
| | AVLRAFWRADGDALDG | | TGCCGCGAATGCGGTGCTTCGAGCGTTCTGGCGTGCTGACGGCGAC |
| | YMVEHGHGPKKAVDWP | | GCGCTCGATGGCTACATGGTCGAGCATGGCCACGGGCCGAAGAAGG |
| | MPKIQSYGLARLVAPM | | CGGTCGACTGGCCCATGCCAAAGATCCAGAGCTACGGGCTCGCCCG |
| | LPSGIGSAVARMAETK | | TCTCGTCGCTCCGATGCTTCCGAGCGGCATTGGGTCAGCGGTCGCT |
| | WRQTRFEALVRNTEKP | | CGCATGGCCGAGACCAAGTGGCGCCAGACTCGGTTCGAGGCCCTGG |
| | AHYREANPIPIRQQDY | | TCCGAAACACCGAAAAGCCCGCGCACTACCGCGAGGCCAATCCGAT |
| | SLTKEDGRWCLSMMLR | | CCCGATTCGGCAGCAGGACTACTCGTTGACGAAAGAAGACGGTCGC |
| | SGEPRVKLPLDIRDSF | | TGGTGCCTCTCCATGATGCTCCGGTCGGGCGAGCCGCGCGTGAAGC |
| | QRSILENVSEWKGSRA | | TCCCGCTTGACATTCGCGACTCGTTCCAGCGTTCCATCTTGGAGAA |
| | LARKKGESREAYKQRV | | CGTCTCCGAATGGAAGGGCAGCCGCGCGCTCGCGCGCAAGAAGGGC |
| | ASLEAAQRGWEAGELR | | GAGAGCCGCGAGGCGTACAAGCAGCGCGTCGCTTCGCTGGAGGCGG |
| | IEQDSKRKARWYVRMA | | CTCAGCGTGGCTGGGAGGCTGGCGAGCTTCGGATCGAGCAAGACTC |
| | YKRIVDKQADGKRASL | | GAAGCGCAAGGCGCGCTGGTACGTCCGCATGGCCTACAAGCGAATC |
| | HRGIKNFLVCVTDDGQ | | GTCGACAAGCAGGCAGACGGCAAGCGGGCTTCCTTACATCGAGGAA |
| | EWKYEGADIEAFLAQM | | TCAAAAACTTCCTCGTCTGCGTGACCGACGATGGCCAAGAGTGGAA |
| | QARRRQYQRNSLASSR | | GTACGAGGGCGCGGACATCGAGGCGTTCCTCGCGCAGATGCAGGCG |
| | SGHGRRVILRPIDKLQ | | CGTCGCCGTCAGTACCAGCGCAACAGCCTCGCGTCGTCGCGAAGCG |
| | GKADRWRRTKNQTLAR | | GCCACGGCAGGCGCGTCATCTTGCGACCCATCGACAAACTGCAAGG |
| | RLAEWLRDRGVSVLFI | | CAAGGCTGACCGCTGGCGCAGGACCAAGAACCAGACCTTGGCGCGC |
| | EDLSGIRSGEPEKLEG | | AGGCTTGCCGAGTGGCTGCGAGACCGCGGCGTCTCGGTGCTTTTCA |
| | GERIYKRVQEWPFYDA | | TCGAAGACCTCTCTGGGATTCGCTCCGGCGAGCCCGAGAAGCTTGA |
| | GQRIHSCCEELGINVK | | GGGCGGCGAGCGTATCTACAAGCGCGTTCAGGAGTGGCCCTTTTAC |
| | TVSPAYDSQRCPVCGA | | GATGCGGGTCAGCGCATTCACTCCTGCTGCGAAGAGCTAGGGATCA |
| | IDPEHKDLRYWKLSCK | | ACGTCAAGACGGTGAGCCCAGCCTACGACTCGCAGCGTTGCCCTGT |
| | SCGARRDLDVAAAYNV | | CTGCGGCGCCATTGACCCAGAGCACAAGGACCTCCGCTACTGGAAG |
| | LARGLAVHGGKGEDYK | | CTCTCGTGCAAGTCTTGCGGCGCTCGTCGCGACCTCGACGTCGCCG |
| | DIGRAKRAAKGKKNAG | | CCGCCTACAACGTCCTCGCGCGCGGGCTTGCGGTCCACGGGGGCAA |
| | | | GGGAGAGGATTACAAGGACATTGGCCGCGCCAAGCGCGCTGCCAAG |
| | | | GGCAAGAAGAATGCTGGTTGA |
| 23 | MYGDSRIEEGNMDALG | 55 | ATGTACGGCGATTCTCGAATCGAGGAAGGAAACATGGACGCGCTCG |
| | KSSRDGTNQHPPCDAA | | GTAAATCGTCCCGAGATGGGACCAATCAGCATCCGCCTTGCGACGC |
| | STAVCETAVIVRATAR | | TGCCTCAACCGCCGTTTGCGAAACGGCCGTCATCGTTCGTGCGACT |
| | MTIDFMAMEEPERIRV | | GCTCGCATGACGATTGACTTCATGGCCATGGAGGAGCCGGAGCGCA |
| | RQLLYEARRNSAAAAN | | TCCGCGTCCGGCAGCTTCTTTACGAGGCACGCAGGAACTCGGCTGC |
| | AVLRAFWRADGDALDG | | TGCCGCGAATGCGGTGCTTCGAGCGTTCTGGCGTGCTGACGGCGAC |
| | YMVEHGHGPKKAVDWP | | GCGCTCGATGGCTACATGGTCGAGCATGGCCACGGGCCGAAGAAGG |
| | MPKIQSYGLARLVAPM | | CGGTCGACTGGCCCATGCCAAAGATCCAGAGCTACGGGCTCGCCCG |
| | LPSGIGSAVARMAETK | | TCTCGTCGCTCCGATGCTTCCGAGCGGCATTGGGTCAGCGGTCGCT |
| | WRQTRFEALVRNTEKP | | CGCATGGCCGAGACCAAGTGGCGCCAGACTCGGTTCGAGGCCCTGG |
| | AHYREANPIPIRQQDY | | TCCGAAACACCGAAAAGCCCGCGCACTACCGCGAGGCCAATCCGAT |
| | SLTKEDGRWCLSMMLR | | CCCGATTCGGCAGCAGGACTACTCGTTGACGAAAGAAGACGGTCGC |
| | SGEPRVKLPLDIRDSF | | TGGTGCCTCTCCATGATGCTCCGGTCGGGCGAGCCGCGCGTGAAGC |
| | QRSILENVSEWKGSRA | | TCCCGCTTGACATTCGCGACTCGTTCCAGCGTTCCATCTTGGAGAA |
| | LARKKGESREAYKQRV | | CGTCTCCGAATGGAAGGGCAGCCGCGCGCTCGCGCGCAAGAAGGGC |
| | ASLEAAQRGWEAGELR | | GAGAGCCGCGAGGCGTACAAGCAGCGCGTCGCTTCGCTGGAGGCGG |
| | IEQDSKRKARWYVRMA | | CTCAGCGTGGCTGGGAGGCTGGCGAGCTTCGGATCGAGCAAGACTC |
| | YKRIVDKQADGKRASL | | GAAGCGCAAGGCGCGCTGGTACGTCCGCATGGCCTACAAGCGAATC |
| | HRGIKNFLVCVTDDGQ | | GTCGACAAGCAGGCAGACGGCAAGCGGGCTTCCTTACATCGAGGAA |
| | EWKYEGADIEAFLAQM | | TCAAAAACTTCCTCGTCTGCGTGACCGACGATGGCCAAGAGTGGAA |
| | QARRRQYQRNSLASSR | | GTACGAGGGCGCGGACATCGAGGCGTTCCTCGCGCAGATGCAGGCG |
| | SGHGRRVILRPIDKLQ | | CGTCGCCGTCAGTACCAGCGCAACAGCCTCGCGTCGTCGCGAAGCG |
| | GKADRWRRTKNQTLAR | | GCCACGGCAGGCGCGTCATCTTGCGACCCATCGACAAACTGCAAGG |
| | RLAEWLRDRGVSVLFI | | CAAGGCTGACCGCTGGCGCAGGACCAAGAACCAGACCTTGGCGCGC |
| | EDLSGIRSGEPEKLEG | | AGGCTTGCCGAGTGGCTGCGAGACCGCGGCGTCTCGGTGCTTTTCA |
| | GERIYKRVQEWPFYDA | | TCGAAGACCTCTCTGGGATTCGCTCCGGCGAGCCCGAGAAGCTTGA |
| | GQRIHSCCEELGINVK | | GGGCGGCGAGCGTATCTACAAGCGCGTTCAGGAGTGGCCCTTTTAC |
| | TVSPAYDSQRCPVCGA | | GATGCGGGTCAGCGCATTCACTCCTGCTGCGAAGAGCTAGGGATCA |
| | IDPEHKDLRYWKLSCK | | ACGTCAAGACGGTGAGCCCAGCCTACGACTCGCAGCGTTGCCCTGT |
| | SCGARRDLDVAAAYNV | | CTGCGGCGCCATTGACCCAGAGCACAAGGACCTCCGCTACTGGAAG |
| | LARGLAVHGGKGEDYK | | CTCTCGTGCAAGTCTTGCGGCGCTCGTCGCGACCTCGACGTCGCCG |
| | DIGRAKRAAKGKKNAG | | CCGCCTACAACGTCCTCGCGCGCGGGCTTGCGGTCCACGGGGGCAA |
| | | | GGGAGAGGATTACAAGGACATTGGCCGCGCCAAGCGCGCTGCCAAG |
| | | | GGCAAGAAGAATGCTGGTTGA |
| 24 | MKHQYKPKKCKFIEHR | 56 | ATGAAACACCAGTACAAACCCAAGAAATGCAAGTTCATCGAACACC |
| | AVKFDRETGNPKLDAS | | GTGCAGTAAAGTTCGACCGGGAAACCGGCAATCCGAAACTGGATGC |
| | GAEIPFTENRTAVCKI | | AAGCGGGGCCGAAATTCCGTTCACCGAAAACCGTACCGCGGTGTGC |
| | NPKSVDPRLLETFDAS | | AAGATTAACCCGAAGTCCGTCGATCCGAGACTCCTGGAAACCTTCG |
| | KETINDILANMSEHWF | | ATGCCTCCAAGGAAACAATCAACGACATCCTCGCCAACATGTCCGA |
| | DVYTVESGVKNDMKKF | | ACACTGGTTCGATGTCTACACGGTCGAATCCGGTGTCAAGAACGAC |
| | TIMDLYAGAVPGDILK | | ATGAAGAAGTTCACCATCATGGACCTCTATGCCGGCGCAGTCCCTG |
| | GEFTLVHGRKRVLVKK | | GGGACATCCTGAAAGGCGAATTCACCCTCGTCCACGGAAGGAAGCG |
| | TITGYVTRELMAPQED | | CGTACTGGTGAAGAAGACGATTACCGGGTATGTCACCCGTGAACTC |
| | DGFILCDREQFINSLN | | ATGGCGCCCCAGGAGGACGACGGCTTCATCCTGTGCGACCGAGAAC |
| | RKTDKIFGEETSIPAK | | AGTTCATCAACTCCCTCAACCGGAAGACGGACAAGATTTTCGGCGA |
| | WWCDTICGDLDTMLKG | | GGAAACCTCCATTCCGGCAAAGTGGTGGTGCGATACCATCTGCGGC |
| | YAQCVLGMSDTDDGKW | | GACCTTGACACGATGTTGAAGGGTTATGCCCAGTGCGTACTCGGCA |
| | RTAVREVSESIYGNEF | | TGAGCGATACCGACGATGGCAAGTGGAGGACCGCTGTCCGCGAAGT |
| | SRKHAERTIIKLGPQH | | GTCCGAAAGCATCTACGGCAACGAATTCTCCCGGAAACATGCCGAG |
| | LRHVNGLMPDTSVIQW | | CGAACCATCATCAAGCTCGGCCCTCAGCACCTCAGGCACGTAAACG |
| | PISCKICGENATITEP | | GCCTGATGCCGGACACTTCCGTCATCCAATGGCCCATCTCGTGCAA |
| | DFAKEPKLKRLYLASM | | GATCTGCGGTGAGAATGCCACAATCACCGAACCCGACTTCGCCAAG |
| | KAFERIVKESFPKKNV | | GAACCTAAACTCAAACGACTGTACCTGGCCTCCATGAAGGCATTCG |
| | FKPNIPMLPRDSVKKL | | AGCGCATCGTGAAGGAATCGTTCCCAAAGAAGAACGTGTTCAAGCC |
| | DGYYNYSAELLYIPGP | | GAACATCCCGATGCTGCCCAGGGATTCCGTCAAGAAACTGGACGGC |
| | KKASRFRVEFRAKSDR | | TACTACAACTATTCCGCCGAACTCCTCTATATCCCCGGTCCCAAGA |
| | TGNDYYPKDLFKYTSE | | AGGCAAGTCGCTTCCGTGTCGAATTCCGGGCAAAGTCCGACCGTAC |
| | CIIPRFSMLKSTGAMT | | CGGGAACGACTACTACCCGAAGGACCTGTTCAAGTACACCTCCGAG |
| | LNIPYTVPCQKPFMSQ | | TGCATAATCCCGCGCTTCTCCATGCTGAAATCCACCGGGGCCATGA |
| | DAEINWDAGLGIDLGY | | CACTCAACATACCGTACACCGTCCCGTGCCAGAAGCCCTTCATGTC |
| | ARFAMVLSKPASKYPG | | CCAGGATGCCGAAATCAACTGGGACGCCGGCCTCGGCATCGACCTC |
| | MVNWNEALDWFSKKYG | | GGATATGCAAGGTTCGCCATGGTGCTCTCGAAACCCGCTTCCAAGT |
| | LDVLNAHCSKATRKEI | | ATCCCGGAATGGTCAACTGGAACGAAGCCCTTGACTGGTTCTCCAA |
| | EDMIAEERDGKATMGA | | GAAGTATGGCCTCGATGTCCTCAATGCCCACTGCTCCAAGGCGACC |
| | IFLLGVRDGNPPDIQH | | CGGAAGGAAATCGAAGACATGATTGCCGAGGAACGGGATGGAAAGG |
| | DWRPSHDPMATLFTRM | | CCACCATGGGCGCCATCTTCCTCCTCGGGGTACGCGACGGGAACCC |
| | ERRTDKDGSPFYSEQQ | | TCCAGACATCCAGCACGACTGGCGCCCGTCCCATGACCCCATGGCC |
| | LAIIGHTKTFRIQMRQ | | ACCCTGTTCACCAGGATGGAACGCAGGACCGACAAGGACGGCTCCC |
| | IFANRIEYYHRQSEWD | | CGTTCTACTCCGAACAGCAGCTCGCCATCATCGGCCACACCAAGAC |
| | LNHSEEQVFARESEVA | | CTTCCGCATCCAGATGCGCCAGATCTTCGCCAACCGCATCGAATAC |
| | KALAARYDFLNESIRC | | TACCACCGCCAGTCCGAATGGGACCTCAACCATTCCGAGGAACAGG |
| | ITQRFISDILTSDGAF | | TGTTCGCCAGGGAATCCGAGGTCGCCAAGGCCCTTGCCGCAAGGTA |
| | RPAFIAMEDLNLNELE | | CGACTTCCTTAACGAATCCATCCGCTGCATTACCCAGAGGTTCATT |
| | KDSSFKSLYMTITGDW | | TCCGACATCCTGACATCTGATGGGGCGTTCAGGCCGGCGTTCATCG |
| | GIDPRQDYKVSVRKGR | | CCATGGAGGACCTGAACCTCAACGAGCTGGAGAAGGACAGCTCCTT |
| | TVAEITYPEGKKPPRP | | CAAGTCCCTATACATGACAATCACGGGAGACTGGGGCATCGATCCC |
| | AQFPKVFPATEHWNTP | | CGCCAGGATTACAAGGTCTCCGTCCGGAAGGGACGCACCGTCGCAG |
| | ARISAKGQTIVIACTP | | AAATCACATATCCCGAGGGCAAGAAGCCCCCCAGGCCCGCGCAGTT |
| | TSKGTVAMARDSIECY | | CCCCAAGGTGTTCCCGGCTACCGAGCACTGGAACACCCCCGCTAGG |
| | TKKALHIALIKHDVER | | ATCTCCGCCAAGGGACAGACCATCGTCATCGCGTGCACCCCTACCA |
| | LCTHMGILFREVSAKF | | GCAAGGGAACGGTGGCCATGGCACGCGACAGCATCGAGTGCTACAC |
| | TSQTCDCCGNAKAVSH | | CAAGAAGGCGCTCCATATCGCCCTCATCAAGCACGATGTCGAGCGC |
| | DPSENGFDPCASMRAM | | CTGTGCACCCACATGGGCATCCTGTTCCGCGAGGTATCCGCCAAGT |
| | KEGKNFRFKRTFICGN | | TCACATCCCAGACATGCGACTGCTGCGGAAACGCCAAGGCGGTATC |
| | PACPMCQVSVNADSNA | | CCATGACCCGTCTGAAAATGGTTTCGACCCCTGTGCCTCGATGCGG |
| | ASVICHMVRNGKSDYF | | GCCATGAAGGAAGGGAAGAACTTCCGCTTCAAGCGTACCTTCATCT |
| | KDKRAKFKAPKVQKET | | GCGGCAACCCGGCGTGCCCGATGTGCCAGGTCTCCGTCAATGCCGA |
| | KKSSKSKKDK | | CAGCAACGCGGCATCCGTCATCTGCCACATGGTCAGGAACGGGAAA |
| | | | TCCGACTATTTCAAGGACAAGCGTGCCAAGTTCAAGGCACCGAAGG |
| | | | TCCAAAAGGAGACAAAGAAATCATCTAAGTCCAAGAAGGACAAGTA |
| | | | G |
| 25 | MAKGTKNTDILYRSEK | 57 | ATGGCCAAAGGTACCAAGAATACTGATATTCTCTATCGGTCCGAAA |
| | FELFWNRRPVCAPTAE | | AGTTTGAATTGTTTTGGAATCGTAGGCCGGTTTGTGCGCCAACCGC |
| | ELALLTITSENLRTVW | | AGAAGAGCTGGCCTTGCTCACAATCACCAGTGAAAACTTGCGGACA |
| | NEAWRARMDAYENFFK | | GTATGGAACGAAGCCTGGCGTGCCCGCATGGATGCTTACGAAAATT |
| | PIYEKIGTAKKLQDEA | | TCTTCAAGCCTATTTACGAAAAAATCGGGACAGCAAAAAAGCTTCA |
| | LVKGLYGELRDAFKQH | | AGACGAGGCTTTGGTAAAAGGGCTTTATGGAGAACTTCGCGATGCC |
| | GVSLYDQINALTPRRK | | TTCAAGCAGCATGGTGTGAGTTTGTACGACCAGATTAACGCACTAA |
| | ADPAFASIPRNWQEET | | CTCCGCGCCGTAAGGCTGACCCTGCCTTTGCCAGTATCCCGCGTAA |
| | LDCLDASFKSFFALRK | | CTGGCAGGAAGAAACGCTGGATTGCCTGGATGCTTCGTTCAAATCG |
| | NGDADAKQPFARETPG | | TTTTTTGCCCTTCGCAAAAACGGCGATGCGGATGCCAAGCAACCTT |
| | FFCKIPGRYGFSFDGE | | TTGCCCGTGAAACGCCCGGATTTTTCTGTAAAATCCCGGGGCGTTA |
| | NITISFAGLGQRIVCQ | | CGGATTTTCGTTTGATGGTGAAAACATCACCATCAGTTTTGCGGGC |
| | VPDHQRERFGNSLRLK | | TTGGGTCAGCGAATTGTTTGTCAGGTACCCGACCATCAGCGTGAAA |
| | KFEIYRDERDLSKPGS | | GGTTTGGAAATTCCCTGCGTTTGAAAAAGTTTGAGATTTACCGCGA |
| | FWISVAYEIPKPPEKP | | TGAGCGCGACCTTTCAAAACCAGGGAGCTTCTGGATTTCCGTTGCC |
| | VTPDNTVYLALGASYL | | TACGAAATCCCAAAGCCGCCCGAAAAGCCTGTTACTCCTGATAACA |
| | GMVCPKGEFYFRLPRP | | CGGTGTACTTGGCTCTTGGTGCATCTTACCTTGGCATGGTTTGCCC |
| | DFHWKPLVDQVQERLK | | GAAGGGTGAGTTTTACTTCCGCTTGCCTCGTCCGGATTTTCACTGG |
| | NVTKGSRKWKKRISAR | | AAGCCATTAGTTGACCAAGTGCAGGAACGGCTGAAAAACGTCACTA |
| | WRMFDILGKQQKQGQY | | AGGGTTCCCGGAAATGGAAAAAGAGGATATCAGCCCGGTGGCGGAT |
| | ELIQEELLSQGVHFVI | | GTTTGACATTCTGGGCAAACAGCAAAAACAAGGCCAGTACGAACTG |
| | TDLVVRSKTGALADAS | | ATCCAGGAAGAACTGCTTTCACAGGGTGTCCATTTTGTCATCACCG |
| | KPERGGAPTGANWSAQ | | ACTTGGTGGTGCGCAGTAAAACTGGAGCTTTGGCCGATGCTTCCAA |
| | NTGWIANLVAKLAEKA | | ACCGGAGCGAGGCGGAGCGCCAACCGGTGCAAATTGGTCAGCCCAA |
| | KEHGGIVVKREPSQLS | | AATACGGGCTGGATTGCCAATTTGGTAGCCAAGCTGGCCGAAAAAG |
| | PQERKMHPGERKIIIA | | CCAAGGAGCACGGCGGTATTGTTGTTAAGCGCGAGCCATCGCAGCT |
| | RRMREAFLADQQ | | TTCTCCCCAAGAACGGAAAATGCACCCCGGCGAACGCAAAATCATC |
| | | | ATTGCCCGAAGGATGCGAGAAGCGTTTCTTGCCGATCAACAGTAA |
| 26 | METQYYKVAAYPMKIR | 58 | ATGGAAACACAATATTATAAGGTTGCAGCATACCCGATGAAAATCC |
| | LYPTKEQAKTIDSWLL | | GGCTGTACCCCACAAAGGAACAGGCGAAGACCATAGACAGCTGGCT |
| | GLQKAYNMTLYALKEG | | TCTGGGCTTGCAGAAGGCATATAATATGACGCTGTACGCGCTGAAA |
| | VPELRQKSKDGSTEFP | | GAGGGCGTGCCGGAACTTCGACAAAAGTCAAAGGACGGTTCAACAG |
| | NWKYIGSKAWLDSLRE | | AATTTCCAAATTGGAAGTACATCGGCAGTAAAGCATGGCTGGATTC |
| | RSSYVASVPGGCLSSS | | TTTGCGGGAAAGAAGCAGTTATGTTGCAAGTGTCCCCGGTGGCTGT |
| | VGGALGADIKKAWESQ | | CTTTCTTCTAGCGTTGGCGGCGCGTTGGGGGCGGATATTAAAAAAG |
| | GKLPVDAWFKATDAKG | | CTTGGGAAAGTCAGGGAAAGCTCCCGGTTGACGCATGGTTCAAAGC |
| | HSIIRWYSDSRPRKSC | | GACGGATGCAAAAGGACATTCCATTATTCGCTGGTATTCGGATAGC |
| | FFQIEANRFTRTNQSV | | AGACCCCGAAAAAGTTGTTTTTTCCAAATTGAGGCAAACAGGTTCA |
| | YITLRKDFTIKARGWN | | CAAGAACAAATCAGAGTGTGTATATAACACTGCGGAAGGATTTCAC |
| | DKIRFAADSTESFFER | | AATCAAAGCAAGAGGCTGGAACGACAAGATACGTTTTGCAGCGGAT |
| | YRDGNDVFSFRISRDN | | TCAACGGAAAGCTTTTTTGAAAGATACCGGGACGGCAATGATGTTT |
| | CGDYYAVITLKDVYRP | | TCAGTTTCAGAATTAGTAGAGATAACTGTGGCGATTATTATGCAGT |
| | FNVEPERRGIGIDAGV | | GATTAGACTGAAGGATGTTTACCGACCGTTTAACGTGGAACCGGAA |
| | NAMATDSDGVSYENPR | | CGGAGGGGCATTGGAATTGATGCTGGCGTAAATGCCATGGCAACAG |
| | IKKKNETLKAEFGRQM | | ATTCTGATGGCGTTTCTTATGAGAACCCTCGTATCAAAAAGAAAAA |
| | ARRYGIKNEQFRKERK | | TGAGACGTTAAAGGCGGAATTTGGGAGGCAAATGGCCCGACGGTAT |
| | ETRKYNTLHNEEIANQ | | GGCATCAAAAATGAACAATTCCGAAAAGAGCGAAAAGAGACTCGAA |
| | TVEPRCISPSKRYLKA | | AGTATAATACATTGCACAACGAGGAGATTGCGAATCAAACGGTTGA |
| | QVKLSELERKVRRQRD | | GCCAAGATGTATTTCTCCATCAAAACGCTACTTGAAAGCACAGGTA |
| | LIQHTYTARIIAKANL | | AAGCTATCTGAGCTGGAACGTAAGGTAAGACGACAGCGGGACCTTA |
| | VAIENLNVKGMMGNSN | | TTCAGCATACCTACACTGCGCGGATTATAGCGAAAGCAAATCTAGT |
| | LADSLSDAAMSEFLRK | | AGCGATTGAAAATCTGAATGTGAAGGGTATGATGGGCAATTCCAAT |
| | LKYKAQWSGGEYHAIG | | TTGGCAGATAGCCTGTCGGATGCGGCAATGTCAGAGTTCTTGAGGA |
| | TFTASTERCAQCGYVL | | AGCTGAAATATAAGGCGCAGTGGTCAGGCGGAGAATATCATGCAAT |
| | QGTEKLTLADRIFTCP | | CGGGACATTTACCGCTTCCACAGAGCGCTGTGCCCAATGCGGCTAT |
| | ICGNTDDRDANAAKSI | | GTCTTGCAGGGAACGGAAAAGCTCACGCTTGCCGACCGTATTTTTA |
| | LEIAEEEIKEGIPSAD | | CCTGCCCGATTTGCGGAAACACGGATGACCGGGATGCCAATGCAGC |
| | TVKKPKEKKKKTYPDK | | AAAATCCATTTTGGAAATCGCGGAAGAAGAAATCAAAGAGGGCATC |
| | PIGKNYPDVFTHFSEE | | CCATCTGCGGATACCGTCAAAAAGCCAAAGGAAAAGAAAAAGAAAA |
| | LVQQHKNPFVIVNDRQ | | CCTACCCGGATAAGCCAATCGGGAAAAATTATCCTGATGTATTTAC |
| | EVLDDAQGYGYSDRQS | | GCATTTCTCAGAAGAACTTGTTCAACAGCATAAAAATCCGTTTGTA |
| | AQKFWTHKMKTQQKQ | | ATTGTAAATGATAGGCAAGAAGTATTGGATGATGCCCAGGGATATG |
| | | | GGTACAGTGACAGACAGAGTGCCCAGAAATTCTGGACACATAAAAT |
| | | | GAAAACACAACAAAAACAGTAA |
| 27 | METQYYKVAAYPMKIR | 59 | ATGGAAACACAATATTATAAGGTTGCAGCATACCCGATGAAAATCC |
| | LYPTKEQAKTIDSWLL | | GGCTGTACCCCACAAAGGAACAGGCGAAGACCATAGACAGTTGGCT |
| | GLQKAYNMTLYALKEG | | TCTGGGCTTGCAGAAGGCATATAATATGACGCTGTACGCACTGAAA |
| | VPELRQKSKDGSTEFP | | GAGGGCGTGCCGGAACTTCGACAAAAGTCAAAGGACGGTTCAACAG |
| | NWKYIGSKAWLDSLRE | | AATTTCCAAATTGGAAGTACATCGGCAGTAAAGCGTGGCTGGATTC |
| | RSSYVASVPGGCLSSS | | TTTGCGGGAAAGAAGCAGTTATGTTGCAAGTGTCCCCGGTGGCTGT |
| | VGGALGADIKKAWESQ | | CTTTCTTCTAGCGTTGGCGGCGCGTTGGGGGCGGATATTAAAAAAG |
| | GKLPVDAWFKATDAKG | | CTTGGGAAAGTCAGGGAAAGCTCCCGGTTGACGCATGGTTCAAAGC |
| | HSIIRWYSDSRPRKSC | | GACGGATGCAAAAGGACATTCCATTATTCGCTGGTATTCGGATAGC |
| | FFQIEANRFTRTNQSV | | AGACCCCGAAAAAGTTGTTTTTTCCAAATTGAGGCAAACAGGTTCA |
| | YITLRKDFTIKARGWN | | CAAGAACAAATCAGAGTGTGTATATAACACTGCGGAAGGATTTCAC |
| | DKIRFAADSTESFFER | | AATCAAAGCAAGAGGCTGGAACGACAAGATACGTTTTGCAGCGGAT |
| | YRDGNDVFSFRISRDN | | TCAACGGAAAGCTTTTTTGAAAGATACCGGGACGGCAATGATGTTT |
| | CGDYYAVITLKDVYRP | | TCAGTTTCAGAATTAGTAGAGATAACTGTGGCGATTATTATGCAGT |
| | FNVEPERRGIGIDAGV | | GATTAGACTGAAGGATGTTTACCGACCGTTTAACGTGGAACCGGAA |
| | NAMATDSDGVSYENPR | | CGGAGGGGCATTGGAATTGATGCTGGCGTAAATGCCATGGCAACAG |
| | IKKKNETLKAEFGRQM | | ATTCCGATGGCGTTTCTTATGAGAACCCTCGTATCAAAAAGAAAAA |
| | ARRYGIKNEQFRKERK | | TGAGACGTTAAAGGCGGAATTTGGGAGGCAAATGGCCCGACGGTAT |
| | EARKYNTLHNEEIANQ | | GGCATCAAAAATGAACAATTCCGAAAAGAACGAAAAGAGGCTCGAA |
| | TAEPRYISPSKRYLKA | | AGTATAATACATTGCATAATGAGGAGATTGCGAATCAAACGGCTGA |
| | QVKLSELERKVRRQRD | | GCCAAGATATATTTCTCCATCAAAACGCTACTTGAAAGCACAGGTA |
| | LVQHTYTARIIAKANL | | AAGCTATCTGAGCTGGAACGTAAGGTAAGACGACAGCGGGACCTTG |
| | VAIENLNVKGMMGNSN | | TTCAGCATACCTACACTGCGCGGATTATAGCGAAAGCAAATCTAGT |
| | LADSLSDAAMSEFLRK | | GGCGATTGAAAATCTGAATGTGAAGGGTATGATGGGCAATTCCAAT |
| | LKYKAQWSGGEYHAIG | | TTGGCAGATAGCCTGTCAGATGCGGCAATGTCAGAGTTCTTGAGGA |
| | TFTASTERCAQCGYVL | | AGCTGAAATATAAGGCGCAGTGGTCAGGCGGAGAATATCATGCAAT |
| | QGTEKLTLADRIFTCP | | CGGGACATTTACCGCTTCCACAGAGCGCTGTGCCCAATGCGGCTAT |
| | ICGNTDDRDANAAKTI | | GTCTTGCAGGGAACGGAAAAGCTCACGCTTGCCGACCGTATTTTTA |
| | LEIAEEEIKEGIPSAD | | CCTGCCCGATTTGCGGGAACACGGATGACCGGGATGCCAATGCAGC |
| | TVKKPKEKKKKTYPDK | | AAAAACCATTTTGGAAATCGCGGAAGAAGAAATCAAAGAGGGCATC |
| | PIGKNYPDVFTHFSEE | | CCATCTGCGGATACCGTCAAAAAGCCAAAGGAAAAGAAAAAGAAAA |
| | LVQQHKNPFVIVNDRQ | | CCTACCCGGATAAGCCAATCGGGAAAAATTATCCTGATGTATTTAC |
| | EVLDDAQGYGYSDRQS | | GCATTTCTCAGAAGAACTTGTTCAACAGCATAAAAATCCGTTTGTA |
| | AQKFWTHKMKTQQKQ | | ATTGTAAATGATAGGCAAGAAGTATTGGATGATGCCCAGGGATATG |
| | | | GGTACAGTGACAGACAGAGTGCCCAGAAATTCTGGACACATAAAAT |
| | | | GAAAACACAACAAAAACAGTAA |
| 28 | MEVQYNKIDVYPMKIR | 60 | GTGGAAGTTCAGTACAATAAAATTGATGTTTACCCGATGAAAATAA |
| | LYPTKEQAKTIDGWLM | | GGCTTTACCCTACAAAAGAACAGGCAAAAACAATCGATGGGTGGCT |
| | GLQKAYNMTLYALKEG | | AATGGGATTGCAAAAGGCGTATAACATGACGCTGTATGCCCTGAAA |
| | VPELRQKSKSGDAEFP | | GAGGGGGTGCCGGAGCTTCGCCAAAAATCTAAGAGTGGAGATGCAG |
| | NWKYIGKREWLDELRK | | AATTCCCCAACTGGAAGTATATTGGAAAACGGGAGTGGCTGGATGA |
| | NSPCVANVPGGCLSST | | GCTGAGAAAGAATAGCCCGTGTGTGGCTAATGTGCCTGGGGGGTGC |
| | VGGALGADMRKAWESQ | | TTGTCATCTACGGTTGGCGGCGCACTGGGGGCAGATATGCGGAAGG |
| | GKLPVDAWFRATDANG | | CATGGGAGAGCCAGGGAAAACTGCCTGTGGATGCTTGGTTTCGTGC |
| | RHIVRWYSDAKPRKSC | | AACTGACGCAAATGGGCGACATATTGTACGCTGGTATTCGGACGCT |
| | FFQIEAGKISREKQSV | | AAACCAAGAAAAAGTTGCTTTTTCCAGATTGAGGCGGGGAAGATAA |
| | YITLRKDFRVKARGWN | | GCAGAGAAAAGCAAAGTGTGTATATTACACTACGTAAGGATTTTCG |
| | DKIRFSEESDEGFFEK | | AGTAAAAGCGCGGGGGTGGAATGACAAAATCCGTTTTTCAGAGGAG |
| | YRDSKKVLSLRVSRDN | | TCAGATGAAGGCTTTTTTGAGAAATACCGGGACAGTAAGAAAGTGC |
| | CGDYFATITLKDVYRP | | TTAGTTTGAGAGTGAGTAGGGATAATTGCGGAGATTATTTTGCCAC |
| | TKVEAERKGVGIDVGV | | TATCACATTAAAGGATGTATATCGACCGACAAAGGTTGAAGCTGAA |
| | RAMATDSDGRVYENPR | | AGAAAAGGAGTCGGAATTGATGTTGGGGTTCGCGCAATGGCAACGG |
| | IKQKFEDKKTELGRQL | | ATTCGGATGGGAGGGTTTACGAGAACCCCCGAATAAAGCAAAAATT |
| | SRRYGAKNKQFRQDCK | | TGAGGATAAAAAAACGGAGTTGGGGCGACAGCTGTCCCGACGCTAT |
| | EARSFNRLHESEINEK | | GGGGCGAAGAACAAGCAATTCCGGCAGGATTGCAAAGAGGCGCGAA |
| | LVVPKTVLPSRRYNQA | | GCTTTAATAGGTTGCACGAGTCAGAAATCAATGAAAAGCTTGTAGT |
| | QLKLSKLERKMRRKRD | | TCCAAAGACGGTTTTGCCATCGAGGCGTTATAATCAAGCACAATTG |
| | MAQHVYSAEVVRKASL | | AAGCTGTCTAAATTGGAACGTAAAATGCGGCGAAAACGAGATATGG |
| | VAIENLNVKGMMADSN | | CGCAGCATGTCTATTCAGCAGAAGTTGTCAGGAAGGCAAGTTTGGT |
| | LADRLSDAAMSELLRK | | TGCCATTGAAAACTTGAATGTGAAGGGAATGATGGCCGATTCCAAT |
| | LKYKAEWSGASYHAIG | | CTCGCAGACAGATTGTCCGATGCGGCTATGTCTGAGCTTTTGCGCA |
| | TFTASTQRCAKCGYIL | | AACTCAAATACAAAGCGGAGTGGTCGGGAGCCAGTTACCATGCGAT |
| | RGENKLARDDSVFVCP | | TGGTACATTTACTGCATCAACACAGCGGTGTGCGAAATGTGGATAT |
| | VCRNVDGRDANAAKSI | | ATCCTGCGGGGAGAAAATAAATTGGCGCGTGATGATTCCGTTTTTG |
| | LQVAQKEISEGVPSAD | | TATGCCCGGTCTGCAGAAATGTGGATGGCCGGGACGCTAACGCAGC |
| | LIKKLEVKKTKTYPDK | | AAAATCGATTTTGCAAGTTGCACAGAAGGAAATTTCCGAGGGGGTT |
| | PIGKAFPNVFTHFSEE | | CCTTCGGCGGATTTGATAAAGAAATTGGAGGTGAAGAAAACAAAAA |
| | YKKQKRNPFVIVDENK | | CCTACCCGGATAAGCCTATTGGTAAGGCTTTTCCAAATGTTTTTAC |
| | NVLNDAQGYGYRNRQS | | CCATTTTAGCGAGGAGTATAAGAAGCAAAAGAGAAATCCATTTGTA |
| | AQKFWAHKMNQ | | ATTGTTGATGAGAATAAAAATGTCCTGAATGATGCCCAAGGATATG |
| | | | GGTATCGTAACAGACAGAGTGCGCAGAAATTCTGGGCACATAAAAT |
| | | | GAACCAATAA |
| 29 | METQYYKVAAYPMKIR | 61 | ATGGAAACACAATATTATAAGGTTGCAGCATACCCGATGAAAATCC |
| | LYPTKEQAKTIDSWLL | | GGCTGTACCCCACAAAGGAACAGGCGAAGACCATAGACAGCTGGCT |
| | GLQKAYNMTLYALKEG | | TCTGGGCTTGCAGAAGGCATATAATATGACGCTGTACGCACTGAAA |
| | VPEEPEEPEEPGEKKP | | GAGGGTGTGCCGGAAGAACCGGAAGAACCGGAAGAACCGGGCGAGA |
| | ELRRKSEDGSTEFPNW | | AGAAGCCAGAACTGCGCCGAAAATCAGAGGATGGTTCAACGGAGTT |
| | KYIGSGDWIERLERKN | | CCCCAACTGGAAGTATATCGGAAGTGGGGACTGGATTGAGCGGCTG |
| | QSLKGIPHDAYYNAVG | | GAAAGGAAAAACCAGTCTCTCAAAGGAATCCCGCATGATGCGTACT |
| | GMLSVDMKKAWESQGK | | ATAATGCCGTCGGCGGAATGCTTTCGGTTGACATGAAGAAGGCGTG |
| | LPVDKWFQARDEKGHL | | GGAAAGTCAGGGGAAATTACCTGTTGATAAATGGTTTCAGGCCAGG |
| | IVRWYNKGKTRSSAYL | | GATGAGAAGGGCCACTTGATTGTACGATGGTATAACAAGGGGAAGA |
| | QVEARKIVQQGKSVFI | | CGAGAAGTTCCGCCTACCTTCAGGTTGAAGCCAGAAAAATTGTTCA |
| | TLQKGFSVKARGWNEK | | ACAGGGCAAGAGCGTTTTTATAACCCTGCAAAAAGGGTTCAGCGTC |
| | IRFSEDLTQSFFDKYQ | | AAGGCGAGGGGCTGGAATGAAAAAATTCGGTTTTCAGAGGATTTGA |
| | GDKRKVGVRISRDNCG | | CGCAAAGCTTTTTTGACAAATATCAGGGGGACAAAAGAAAAGTAGG |
| | DYYAVISLKDVYRPVK | | TGTGAGAATCAGCAGGGATAATTGCGGTGACTATTACGCTGTAATT |
| | VEAERRSVGVDAGTRV | | TCACTGAAAGATGTTTATCGCCCTGTTAAAGTAGAAGCAGAGAGAA |
| | MATDSDGMTYENPRIK | | GAAGCGTCGGTGTTGATGCGGGAACAAGAGTAATGGCGACAGATTC |
| | KRNEAEKAELDRQLAR | | CGACGGCATGACTTATGAGAATCCGCGGATAAAGAAAAGAAATGAA |
| | RFGARNEQFRAECKAA | | GCTGAAAAGGCGGAACTGGACCGACAGCTGGCACGGCGATTTGGAG |
| | RKYNKSHQEEIAEKVV | | CCAGAAACGAACAGTTCCGTGCGGAATGTAAAGCAGCTCGCAAATA |
| | ELKRVLPSKRYLKAQL | | CAACAAGTCGCATCAAGAAGAAATTGCAGAAAAAGTGGTTGAGCTC |
| | KLSKLERKAARQRDMV | | AAGAGAGTTTTACCATCGAAGCGCTACTTAAAGGCACAGTTAAAAT |
| | QHIHTAQIVAKANLVA | | TGTCCAAATTGGAGCGGAAGGCGGCACGACAGCGGGATATGGTTCA |
| | IEHLNVEGMKKDSNSA | | GCATATCCATACAGCGCAAATTGTGGCAAAAGCCAATCTCGTTGCC |
| | SSVEDAAMSGLLQKIK | | ATCGAGCACTTAAATGTCGAAGGTATGAAAAAGGATTCAAATTCTG |
| | YKAQWSGGNYHSIGTF | | CAAGCAGTGTAGAAGATGCAGCAATGTCGGGACTTCTGCAAAAAAT |
| | DPSTQRCAQCGYVLKD | | AAAGTACAAAGCGCAGTGGTCTGGTGGCAACTATCATTCAATAGGG |
| | EEKLKRGDDTFVCPRC | | ACATTTGACCCCTCTACACAGCGGTGTGCGCAATGTGGGTATGTTT |
| | GNVDGRDENAAKSILI | | TGAAGGATGAAGAAAAACTAAAGCGAGGGGACGATACGTTTGTTTG |
| | VAKERIERGLPSADTI | | TCCGCGCTGCGGCAATGTGGATGGCCGAGACGAAAACGCAGCAAAA |
| | KKPKEKKKKTYPDKPI | | TCTATACTGATAGTTGCGAAAGAACGTATTGAAAGAGGACTTCCAT |
| | GKNYPGVFTHFSEEFR | | CTGCGGACACCATCAAGAAGCCAAAGGAAAAGAAAAAGAAAACCTA |
| | QQYKNPFVIVNDKQEV | | CCCGGATAAGCCAATCGGGAAAAACTATCCTGGTGTATTCACGCAT |
| | LDDAQGYGYSDRQSAQ | | TTTTCAGAGGAATTTAGACAACAATATAAAAATCCGTTTGTAATTG |
| | KFWTHKMKTQQKQ | | TAAATGATAAGCAGGAAGTATTGGATGATGCCCAGGGGTATGGGTA |
| | | | CAGTGATAGACAGAGTGCCCAGAAATTCTGGACACATAAAATGAAA |
| | | | ACACAACAAAAACAGTAA |
| 30 | MKTIKIKIKLTTDQVQ | 62 | ATGAAAACAATCAAAATCAAAATCAAACTCACCACTGATCAGGTGC |
| | LCDRYLEELTWLWNLT | | AACTGTGCGATCGCTATTTGGAAGAATTGACATGGCTGTGGAATTT |
| | LSNQLHNHCVTWYDWA | | AACACTTTCTAATCAACTACATAATCACTGTGTGACGTGGTACGAT |
| | AKLSANLDKATEKLDK | | TGGGCGGCTAAACTCAGTGCTAATTTGGATAAAGCTACTGAGAAAT |
| | LKPEQQQLIKDYYRTK | | TAGATAAACTTAAACCAGAACAACAGCAGTTAATAAAGGATTATTA |
| | DKPKLSKKEQELVAKF | | CAGGACTAAGGATAAGCCTAAGCTATCTAAAAAAGAACAGGAATTA |
| | DIFARWNSFSLDGIIP | | GTGGCTAAATTTGACATATTTGCTAGATGGAATTCATTTAGTCTAG |
| | VPLRLGNSGYEGLSCQ | | ACGGGATTATTCCCGTTCCTTTACGGCTGGGGAATAGCGGTTATGA |
| | IATGGNYWKRDENINI | | AGGTTTATCCTGTCAGATAGCTACGGGTGGTAACTACTGGAAGAGA |
| | PINTKKGIVHVKGYKL | | GATGAAAATATCAATATCCCAATTAATACCAAGAAAGGTATTGTTC |
| | VKGDKPWQRIEIVPHK | | ACGTTAAAGGATACAAATTAGTTAAAGGTGATAAACCTTGGCAACG |
| | YRTFPGGKFEGRELTT | | AATAGAAATAGTACCCCATAAGTACCGTACATTCCCCGGTGGAAAG |
| | LEKLDNVNGLNTLRAF | | TTTGAAGGTAGGGAACTGACTACTTTAGAGAAACTTGATAACGTGA |
| | QNLPDLQVSSHYIGGL | | ATGGACTGAACACTTTAAGGGCATTCCAAAATTTACCAGACCTACA |
| | LAFFKESWSAFLDPKR | | AGTTTCCTCCCACTATATAGGCGGACTACTGGCATTTTTTAAAGAA |
| | MNSRKPKFKKDSDKIT | | TCATGGTCGGCATTTTTAGACCCTAAAAGGATGAACAGTAGAAAGC |
| | TLSNNQCAPNRIDVNK | | CTAAATTCAAAAAAGACAGTGACAAGATTACAACTTTATCTAACAA |
| | NIVTVTGFSPIAIIDK | | TCAGTGTGCGCCTAACAGAATTGACGTTAATAAAAACATAGTTACT |
| | NWVKRLNLSEVLPRTY | | GTCACTGGGTTTAGTCCTATCGCCATTATTGATAAAAATTGGGTAA |
| | MLTQNPSGYYINIVIA | | AAAGATTGAACTTGTCTGAAGTTCTACCACGCACATATATGTTAAC |
| | HPLHEEKTALVKKLPK | | CCAAAATCCATCAGGGTACTACATTAATATTGTCATTGCTCATCCA |
| | VKKEFGEDSQEYEDIK | | TTACATGAAGAGAAAACCGCGCTGGTAAAGAAATTACCCAAAGTTA |
| | SKIKFLEQQIKEASIV | | AAAAAGAGTTTGGAGAAGATAGTCAAGAGTATGAAGATATCAAGTC |
| | KGKDLSVGIDPGVQAV | | AAAAATCAAATTTCTTGAACAACAAATCAAGGAAGCATCAATAGTT |
| | VSTDHGALFLPNLTRE | | AAAGGCAAAGATTTAAGTGTGGGCATTGACCCCGGAGTACAGGCTG |
| | RVSIHIEELQSRLDNI | | TTGTGTCCACAGATCATGGTGCTTTATTCCTTCCTAACCTTACCAG |
| | ELINDKKWKSLGNKTP | | AGAGCGGGTTTCAATTCATATTGAGGAATTACAATCACGCTTAGAC |
| | RIKTKNETKLQEKISR | | AATATTGAATTAATTAACGATAAAAAGTGGAAAAGCTTGGGCAACA |
| | LHERGANSSNAFNHKL | | AGACCCCTAGAATCAAGACAAAGAATGAAACTAAGTTACAGGAAAA |
| | STRLSRTYEHIAWEDT | | AATAAGCCGTCTACACGAACGTGGGGCTAATTCATCCAATGCTTTT |
| | QINNLLKQVEPKALPE | | AATCATAAGCTATCCACAAGATTATCTCGTACTTATGAACATATTG |
| | GIGYAHNGASAKRGLN | | CCTGGGAAGACACGCAGATTAATAATCTACTAAAACAAGTAGAGCC |
| | WIMRQRCLSDLKAKTK | | AAAAGCATTACCAGAAGGTATAGGATATGCTCACAATGGTGCATCT |
| | QKTENRGGNFHEPPAN | | GCTAAACGCGGTTTGAATTGGATTATGAGACAAAGATGTTTGAGTG |
| | YSSQTCHCCKQKGERR | | ATTTGAAAGCTAAAACCAAACAGAAAACAGAAAATAGAGGTGGCAA |
| | SQHEFICKNSDCKLFD | | CTTCCATGAACCACCTGCTAACTATAGTTCTCAAACTTGTCATTGC |
| | IPQQADTNAARNHKQN | | TGTAAACAAAAGGGAGAACGGCGATCGCAACATGAATTTATCTGCA |
| | GGFELGEVKYHNVKLV | | AAAACTCTGACTGTAAATTATTTGATATTCCCCAGCAAGCTGATAC |
| | YQKPKRFKKKRLTNQ | | TAACGCTGCTAGAAATCATAAGCAGAATGGTGGTTTTGAACTGGGA |
| | | | GAGGTCAAGTATCATAATGTTAAGCTAGTTTATCAAAAGCCTAAAA |
| | | | GATTTAAGAAAAAACGCTTGACAAACCAATAG |
| 31 | MTKAERLRDVAKNRLV | 63 | ATGACCAAGGCTGAGCGCCTTCGCGACGTAGCGAAGAACCGGCTCG |
| | RLERRAQKIGEHLKRK | | TCCGGCTGGAGCGACGTGCGCAAAAGATTGGCGAACATCTAAAGCG |
| | PGDLQAMHHLLHQIEV | | GAAACCGGGCGACCTGCAAGCGATGCACCATCTCCTGCATCAAATC |
| | EYHDVSRNLKDPDWVP | | GAAGTCGAGTACCACGACGTCTCGCGCAATCTAAAGGATCCTGACT |
| | KPKRKREKRNIESTDH | | GGGTTCCTAAGCCAAAGCGCAAGCGAGAGAAGCGCAATATCGAGAG |
| | IPPPTKGDPGVPKHYS | | TACCGACCATATCCCGCCCCCCACGAAGGGCGACCCTGGCGTGCCA |
| | IPKPVPLPVDRIPEDQ | | AAGCACTATTCAATACCGAAGCCCGTGCCTCTGCCGGTTGACCGAA |
| | LKMGWKSSGRSWCSPP | | TCCCGGAAGACCAGCTCAAGATGGGATGGAAGAGCAGCGGACGCTC |
| | FVEVALPPGKEHVVID | | TTGGTGTTCTCCCCCGTTCGTAGAGGTTGCCCTACCGCCCGGGAAA |
| | HLSKFKIDDDREVVRA | | GAGCACGTCGTTATTGACCATCTGTCAAAGTTCAAGATTGACGACG |
| | WAEKEFGSIAVAKEAL | | ATAGGGAAGTCGTGCGTGCCTGGGCCGAGAAAGAGTTTGGGTCGAT |
| | KVGATLSVDAGVWRGL | | CGCGGTTGCTAAAGAAGCCCTTAAGGTTGGCGCTACGCTTTCGGTT |
| | IEQAGKSERFSDLTGE | | GATGCTGGCGTTTGGCGCGGCTTGATTGAGCAGGCGGGTAAGTCAG |
| | ELLADASARRIAMAWH | | AGCGCTTCAGTGACCTCACCGGCGAAGAACTGCTTGCAGACGCCTC |
| | QFEWVKQTAKSITDSA | | TGCGCGGAGGATCGCGATGGCCTGGCACCAATTCGAATGGGTGAAG |
| | PKGVSKDALASKTRAH | | CAAACGGCAAAATCAATCACCGACTCTGCGCCGAAAGGAGTCTCGA |
| | LKSFHTAVNSFKQVTN | | AGGATGCTTTGGCGTCGAAAACAAGGGCACACCTCAAATCGTTTCA |
| | KETGAVELVNKHTNPQ | | CACAGCGGTGAACTCGTTCAAGCAGGTTACGAACAAGGAGACGGGC |
| | FSYLSMEKPVVDADTV | | GCTGTCGAACTTGTGAACAAGCACACCAACCCGCAGTTTTCGTACC |
| | SEQVVDWLSLPVDERF | | TGAGCATGGAAAAGCCGGTAGTCGATGCGGACACCGTAAGCGAGCA |
| | TKDENDPKKRGRITVL | | AGTCGTCGACTGGCTCTCACTCCCGGTCGACGAACGCTTCACCAAA |
| | QKELGAAKRSKHWRGR | | GACGAAAATGATCCGAAGAAGCGGGGGCGAATCACCGTCTTGCAAA |
| | EQTQPWAGKPHWKGTL | | AGGAACTGGGCGCTGCAAAGCGGTCAAAGCACTGGCGAGGCCGCGA |
| | IRKRDALLIYDCCKNG | | ACAGACGCAGCCTTGGGCAGGCAAGCCTCATTGGAAGGGCACGCTC |
| | LALVLCTGGGVRVDVE | | ATCAGGAAGCGCGACGCATTGCTGATTTATGACTGTTGCAAGAATG |
| | SLLNMDGTDLRSDRQL | | GCCTCGCCCTCGTGCTTTGCACAGGCGGGGGAGTGCGTGTCGACGT |
| | LTPNGTGKSAFVLPLV | | GGAATCACTGCTGAATATGGATGGCACTGACCTCCGGTCGGATCGT |
| | PKHDFHRWYAKHVEND | | CAACTCCTTACCCCAAACGGTACCGGAAAGTCGGCGTTCGTGTTGC |
| | NADAPLTKRCIHNTTQ | | CCTTGGTCCCTAAGCATGATTTTCACCGTTGGTATGCGAAGCACGT |
| | FVVIPEHKGHPPQLFI | | CGAAAATGACAATGCAGACGCACCGCTGACCAAGCGATGTATCCAC |
| | RPVLKFYDPGKEIPDT | | AACACCACTCAGTTCGTGGTCATTCCCGAGCACAAAGGGCATCCGC |
| | HAWGAKPQCRYLIGVD | | CGCAACTATTTATTCGACCGGTCCTCAAATTCTACGATCCCGGAAA |
| | RGINSPYFAAVYDTER | | GGAGATTCCCGACACCCACGCTTGGGGAGCGAAACCTCAGTGTCGG |
| | NAIVAIRQGRGRKDEW | | TATCTCATTGGCGTGGACCGCGGCATCAACTCACCCTATTTTGCTG |
| | KNLRNELALAQREHNE | | CTGTCTATGACACGGAACGGAACGCCATCGTAGCCATACGACAGGG |
| | LRNKRGKAKQLAKAMA | | TCGCGGACGAAAGGACGAATGGAAGAATCTTCGAAACGAGCTCGCT |
| | NIRALRKKERGLNKVE | | CTGGCACAGCGCGAACACAACGAACTTCGCAATAAGCGAGGCAAAG |
| | TVESIAELANWAEKEL | | CGAAGCAGCTGGCAAAGGCGATGGCCAACATTCGTGCATTGCGAAA |
| | GACNYCFVIEELQQMN | | GAAGGAGCGAGGGCTGAATAAAGTAGAGACCGTGGAGTCGATTGCC |
| | LRRNNRVKNIAAIKDA | | GAACTCGCGAACTGGGCCGAAAAAGAATTGGGCGCATGCAACTACT |
| | LVNQMRKKGYKYKEKS | | GTTTTGTGATTGAGGAGCTTCAGCAGATGAACCTCAGGCGGAATAA |
| | GKVDGVREESPWHTSA | | TCGGGTTAAGAATATTGCGGCAATCAAGGACGCCCTTGTCAATCAA |
| | VSPFGWWAKSEDVDKD | | ATGCGAAAGAAGGGCTACAAATACAAGGAGAAAAGTGGGAAGGTAG |
| | KRFIGRRVGGHYCRDA | | ACGGCGTTCGCGAAGAATCACCGTGGCATACGAGCGCTGTTTCGCC |
| | EDGRYIRGMYKKPGGK | | CTTTGGTTGGTGGGCGAAGTCAGAAGATGTCGATAAAGACAAGCGG |
| | YGRKVFTLSEDDLRTG | | TTCATCGGGCGTCGCGTCGGCGGACATTACTGCCGCGACGCTGAGG |
| | IRRRSFGSELFWDPHR | | ATGGGCGCTACATCCGAGGAATGTATAAGAAACCAGGCGGCAAGTA |
| | TEFRGKPFPNGVVLNA | | CGGACGAAAAGTCTTCACTCTCTCTGAGGACGATTTGAGAACCGGG |
| | DFVGAFNIAVRPVVKD | | ATTCGACGCCGCTCATTCGGCTCGGAGCTTTTCTGGGACCCGCATC |
| | GKGKGFTAKNMAEVHT | | GGACCGAGTTTCGTGGCAAGCCGTTTCCCAACGGTGTTGTCCTGAA |
| | EFNPTVAIECEIPLYE | | CGCTGACTTTGTCGGGGCGTTCAACATTGCCGTGCGCCCCGTGGTG |
| | FTEVDGDPLGALREW | | AAGGACGGCAAGGGTAAGGGCTTTACTGCGAAGAACATGGCGGAGG |
| | V | | TACACACCGAGTTCAATCCAACGGTGGCTATCGAGTGTGAGATACC |
| | | | CCTATACGAGTTCACCGAAGTGGACGGCGATCCCCTTGGCGCCCTT |
| | | | CGCGAAGTGGTTGTATAA |
| 32 | MSPLERSLRKVGENRL | 64 | TTGAGCCCGCTCGAACGGTCCTTGCGGAAGGTCGGTGAGAATCGCC |
| | ERLRVREEKIRKHIEQ | | TTGAGCGGCTGCGGGTGCGAGAGGAGAAGATTAGGAAGCACATAGA |
| | HPRGKNDHQALHFLLH | | ACAGCACCCCCGCGGTAAGAACGATCATCAGGCTCTCCACTTCTTA |
| | QIEVERNDLYRNLKDP | | TTGCACCAAATCGAGGTCGAGCGTAACGACCTGTACCGAAACCTCA |
| | EYVPKPAKQRRERRQI | | AAGACCCCGAGTACGTGCCCAAACCAGCGAAACAGCGGCGCGAAAG |
| | NVAKPPTRPKKEKGPQ | | ACGGCAGATCAACGTCGCCAAACCCCCGACTCGACCAAAGAAGGAA |
| | PESTKYVIRPPVPGKN | | AAGGGGCCTCAACCAGAGTCGACGAAGTACGTGATCCGTCCACCAG |
| | LPAFASKYEARDTRDD | | TCCCTGGGAAAAACCTTCCTGCCTTTGCTAGCAAGTACGAGGCGCG |
| | SYQDGRSWTSAPYVEV | | AGACACGCGGGACGATTCCTACCAGGACGGTCGCTCATGGACCTCC |
| | ELPILGADKVIQKLMK | | GCACCATATGTTGAAGTCGAACTTCCCATCCTTGGTGCAGACAAAG |
| | FVQKDERSIVRDWATK | | TCATCCAGAAACTGATGAAGTTCGTGCAGAAGGACGAGCGGTCGAT |
| | TYSSIEAAREALLVGA | | CGTGCGCGACTGGGCGACAAAGACGTATAGCTCGATCGAAGCCGCA |
| | QVSEDVSVWRGLLAET | | AGAGAAGCACTCCTTGTCGGGGCACAAGTCTCGGAAGACGTTTCGG |
| | KNAQNFAALSDDQIEA | | TCTGGCGCGGACTCCTCGCAGAAACGAAGAACGCACAGAACTTCGC |
| | AMSKEAKGADLRPRRA | | CGCCCTCTCCGACGATCAGATCGAAGCAGCGATGTCGAAGGAGGCG |
| | ALLVAQRHWVDQTVKA | | AAGGGCGCGGACTTGCGTCCGAGGCGCGCCGCACTGCTGGTCGCAC |
| | IKESAPSGVDKDTLDR | | AGCGCCACTGGGTGGATCAGACCGTCAAAGCAATCAAGGAGTCCGC |
| | RLRAGLRGFHTAANSG | | ACCGTCCGGCGTCGACAAGGACACTCTCGATCGCCGTCTGCGCGCA |
| | KHTNPQFPYLTAEKPV | | GGTCTGAGGGGGTTTCATACTGCGGCCAACTCAGGCAAGCACACGA |
| | VPMESVVQSVLAFLDD | | ACCCGCAGTTCCCATACCTCACCGCAGAGAAGCCGGTAGTCCCGAT |
| | PDDQRYTKDKEDDKKR | | GGAGTCTGTTGTTCAGAGCGTATTGGCCTTTCTCGACGATCCAGAC |
| | HRVTVLQKELGKARPR | | GATCAAAGGTACACGAAGGACAAAGAAGACGACAAGAAGCGCCACC |
| | KRLELQTPKWAGRPTV | | GCGTCACTGTCTTGCAGAAGGAGCTCGGAAAGGCGAGGCCACGAAA |
| | KGTISKRRDAALVWDT | | ACGGTTAGAACTCCAAACGCCGAAATGGGCCGGCAGGCCCACGGTA |
| | SKEANGLCLALPIGGM | | AAAGGAACCATCAGCAAACGGCGCGACGCAGCGCTCGTCTGGGACA |
| | PKIDVEQFIYQDGTSL | | CAAGCAAAGAAGCGAACGGGCTTTGTCTCGCGCTCCCAATCGGGGG |
| | LSDCQIASKTTKKGAA | | CATGCCGAAGATAGACGTCGAGCAGTTCATCTACCAGGATGGGACG |
| | CAVLPLKPKHDFLRWF | | TCGCTCCTGTCCGATTGCCAGATCGCATCGAAAACGACCAAGAAGG |
| | TKHVENHNPDAPLERR | | GCGCGGCTTGCGCAGTCTTGCCGCTCAAGCCCAAGCATGACTTCCT |
| | CLHNTTQFVIVDPEGP | | GCGCTGGTTCACCAAGCACGTCGAGAACCACAATCCCGACGCTCCA |
| | RPRLFVRPVFKFYDPG | | CTGGAACGCAGGTGCCTCCACAACACGACCCAGTTCGTCATAGTCG |
| | KTVPNTHETWKKPDCR | | ACCCAGAAGGGCCGCGCCCACGTCTCTTCGTCCGGCCCGTCTTCAA |
| | YLVGIDRGINYVLRAV | | GTTCTACGACCCCGGCAAGACGGTGCCGAACACGCATGAAACTTGG |
| | VVDTEEKKVIADIGLP | | AAAAAGCCCGACTGCCGCTACCTGGTTGGAATCGACCGAGGCATCA |
| | GRKHEWRMIRDEIAYH | | ATTACGTTCTGCGAGCCGTCGTCGTCGATACTGAAGAGAAGAAGGT |
| | QQMRDLARNTGKHASV | | TATCGCCGATATCGGCTTGCCGGGCAGGAAGCACGAATGGAGGATG |
| | VAKHVRALALARKKDR | | ATCCGTGACGAGATCGCCTACCACCAACAGATGCGTGATCTTGCCC |
| | ALGKFATVEAVAELVK | | GCAACACTGGCAAACACGCGAGCGTCGTGGCCAAGCACGTCCGCGC |
| | KCEQDYGSGNYCFVLE | | CCTCGCGCTCGCGCGCAAGAAGGACCGCGCGCTCGGCAAGTTCGCA |
| | DLDMGAMNLKRNNRVK | | ACAGTCGAAGCCGTCGCAGAACTTGTCAAGAAGTGTGAACAGGACT |
| | HMAVMEEALVNQMRKQ | | ATGGTAGCGGCAACTACTGTTTCGTGCTCGAAGACCTCGACATGGG |
| | GYAYDGRRGRVDGVRH | | GGCGATGAATCTCAAGCGAAACAACAGAGTCAAACACATGGCGGTC |
| | EGAWYTSQVSPFGWWA | | ATGGAGGAGGCCCTCGTCAATCAAATGCGCAAGCAGGGCTATGCCT |
| | KRDEVEEAWKRDKTRP | | ATGACGGGCGTCGCGGTCGGGTGGACGGCGTGAGGCACGAGGGCGC |
| | IGRKVGNWYEMPEPGQ | | TTGGTACACGAGCCAGGTCTCGCCCTTTGGCTGGTGGGCCAAGCGC |
| | DGDRPDTYRKGYWSKP | | GACGAAGTCGAGGAGGCGTGGAAGAGGGACAAGACTCGCCCCATCG |
| | KNAEGKPYGRNRFSVE | | GGCGCAAGGTCGGCAACTGGTACGAGATGCCCGAGCCAGGCCAAGA |
| | PGDEKPDAERRFCWGS | | CGGAGACCGGCCCGACACGTATCGGAAGGGCTACTGGTCGAAACCG |
| | ELFWDPNVKSFKGKEF | | AAGAACGCGGAGGGCAAGCCGTATGGGCGCAACCGCTTCAGCGTCG |
| | PEGVVLDADFVGALNI | | AGCCTGGCGACGAGAAGCCGGACGCTGAGCGGCGCTTCTGCTGGGG |
| | ALRPLVNDGQGKGFKA | | CAGCGAGCTGTTCTGGGATCCGAACGTGAAGTCCTTCAAGGGCAAG |
| | EDMAREHTILNPQFKI | | GAGTTTCCCGAGGGCGTCGTGCTGGACGCCGACTTCGTAGGAGCCC |
| | ACQIPVYEFVEEDGDK | | TCAACATCGCTCTCCGCCCGTTGGTCAACGACGGCCAGGGTAAAGG |
| | WAALRRIML | | CTTCAAGGCCGAGGACATGGCGAGGGAGCACACGATACTAAACCCG |
| | | | CAGTTCAAGATCGCCTGCCAGATACCAGTTTACGAGTTCGTCGAAG |
| | | | AGGACGGCGACAAGTGGGCAGCTCTGCGCCGGATCATGCTATAG |

In some embodiments, a nuclease of the present invention is a nuclease having a specified degree of amino acid sequence identity to one or more reference polypeptides, e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99% sequence identity to the amino acid sequence of any one of SEQ ID NOs: 1-32. Homology or identity can be determined by amino acid sequence alignment, e.g., using a program such as BLAST, ALIGN, or CLUSTAL, as described herein. In some embodiments, a nuclease having a specified degree of amino acid sequence identity to one or more reference polypeptides retains one or more characteristics, e.g., nuclease activity, as the one or more reference polypeptides.

In some embodiments, a nuclease of the present invention comprises a protein with an amino acid sequence with at least about 60%, least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to the reference amino acid sequence. In some embodiments, a nuclease having a specified degree of amino acid sequence identity to one or more reference polypeptides retains one or more characteristics, e.g., nuclease activity, as the reference amino acid sequence.

Also provided is a nuclease of the present invention having enzymatic activity, e.g., nuclease activity, and comprising an amino acid sequence which differs from the amino acid sequences of any one of any one of SEQ ID NOs: 1-32 by no more than 50, no more than 40, no more than 35, no more than 30, no more than 25, no more than 20, no more than 19, no more than 18, no more than 17, no more than 16, no more than 15, no more than 14, no more than 13, no more than 12, no more than 11, no more than 10, no more than 9, no more than 8, no more than 7, no more than 6, no more than 5, no more than 4, no more than 3, no more than 2, or no more than 1 amino acid residue(s), when aligned using any of the previously described alignment methods.

In some embodiments, a nuclease of the present invention comprises a RuvC domain. In some embodiments, a nuclease of the present invention comprises a split RuvC domain or two or more partial RuvC domains. For example, a nuclease comprises RuvC motifs that are not contiguous with respect to the primary amino acid sequence of the nuclease but form a RuvC domain once the protein folds. In some embodiments, the catalytic residue of a RuvC motif is a glutamic acid residue and/or an aspartic acid residue.

In some embodiments, the invention includes an isolated, recombinant, substantially pure, or non-naturally occurring nuclease comprising a RuvC domain, wherein the nuclease has enzymatic activity, e.g., nuclease activity, wherein the nuclease comprises an amino acid sequence having at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to any one of SEQ ID NOs: 1-32.

In some embodiments, a nuclease of the present invention forms a dimer. In some embodiments, the dimer is a homodimer (e.g., a homodimer comprising two identical RuvC domains). In some embodiments, the dimer is a heterodimer (e.g., a heterodimer comprising two non-identical RuvC domains). For example, in some embodiments, a first nuclease polypeptide of SEQ ID NO: 1 forms a homodimer with a second nuclease polypeptide of SEQ ID NO: 1. In other embodiments, a first nuclease polypeptide of SEQ ID NO: 1 forms a heterodimer with a second nuclease polypeptide of any one of SEQ ID NOs: 2-32. In some embodiments, a dimer of the present invention (e.g., a dimer comprising two RuvC domains) is capable of cleaving two target nucleic acid molecules. In some embodiments, a dimer of the present invention (e.g., a dimer comprising two RuvC domains) is capable of cleaving two sites within a single nucleic acid target. In some embodiments, a dimer of the present invention (e.g., a dimer comprising two RuvC domains) is capable of editing two sites within a single nucleic acid target. In some embodiments, a dimer of the present invention (e.g., a dimer comprising two RuvC domains) is capable of introducing an indel at two sites within a single nucleic acid target.

Variants

In some embodiments, the present invention includes variants of a nuclease described herein. In some embodiments, a nuclease described herein can be mutated at one or more amino acid residues to modify one or more functional activities. For example, in some embodiments, a nuclease of the present invention is mutated at one or more amino acid residues to modify its nuclease activity (e.g., cleavage activity). For example, in some embodiments, a nuclease may comprise one or more mutations that increase the ability of the nuclease to cleave a target nucleic acid. In some embodiments, a nuclease is mutated at one or more amino acid residues to modify its ability to functionally associate with an RNA guide. In some embodiments, a nuclease is mutated at one or more amino acid residues to modify its ability to functionally associate with a target nucleic acid.

In some embodiments, a variant nuclease has a conservative or non-conservative amino acid substitution, deletion or addition. In some embodiments, the variant nuclease has a silent substitution, deletion or addition, or a conservative substitution, none of which alter the polypeptide activity of the present invention. Typical examples of the conservative substitution include substitution whereby one amino acid is exchanged for another, such as exchange among aliphatic amino acids Ala, Val, Leu and Ile, exchange between hydroxyl residues Ser and Thr, exchange between acidic residues Asp and Glu, substitution between amide residues Asn and Gln, exchange between basic residues Lys and Arg, and substitution between aromatic residues Phe and Tyr. In some embodiments, one or more residues of a nuclease disclosed herein are mutated to an Arg residue. In some embodiments, one or more residues of a nuclease disclosed herein are mutated to a Gly residue.

A variety of methods are known in the art that are suitable for generating modified polynucleotides that encode variant nucleases of the invention, including, but not limited to, for example, site-saturation mutagenesis, scanning mutagenesis, insertional mutagenesis, deletion mutagenesis, random mutagenesis, site-directed mutagenesis, and directed-evolution, as well as various other recombinatorial approaches. Methods for making modified polynucleotides and proteins (e.g., nucleases) include DNA shuffling methodologies, methods based on non-homologous recombination of genes, such as ITCHY (See, Ostermeier et al., 7:2139-44 [1999]), SCRACHY (See, Lutz et al. 98:11248-53 [2001]), SHIPREC (See, Sieber et al., 19:456-60 [2001]), and NRR (See, Bittker et al., 20:1024-9 [2001]; Bittker et al., 101:7011-6) [2004], and methods that rely on the use of oligonucleotides to insert random and targeted mutations, deletions and/or insertions (See, Ness et al., 20:1251-5 [2002]; Coco et al., 20:1246-50 [2002]; Zha et al., 4:34-9 [2003]; Glaser et al., 149:3903-13 [1992]).

In some embodiments, a nuclease of the present invention comprises an alteration at one or more (e.g., several) amino acids in the nuclease, wherein at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 162, 164, 164, 165, 166, 167, 168, 169, 170, 171, 172, 173, 174, 175, 176, 177, 178, 179, 180, 181, 182, 183, 184, 185, 186, 187, 188, 189, 190, 191, 193, 194, 195, 196, 197, 198, 199, 200, or more amino acids are altered. In one embodiment, the alteration is relative to a parent polypeptide, wherein the alteration comprises one or more substitutions, insertions, deletions, and/or additions in the nuclease relative to the parent polypeptide.

As used herein, a “biologically active portion” is a portion that maintains the function (e.g. completely, partially, minimally) of a nuclease (e.g., a “minimal” or “core” domain). In some embodiments, a nuclease fusion protein is useful in the methods described herein. Accordingly, in some embodiments, a nucleic acid encoding the fusion nuclease is described herein. In some embodiments, all or a portion of one or more components of the nuclease fusion protein are encoded in a single nucleic acid sequence.

Although the changes described herein may be one or more amino acid changes, changes to a nuclease may also be of a substantive nature, such as fusion of polypeptides as amino- and/or carboxyl-terminal extensions. For example, nuclease may contain additional peptides, e.g., one or more peptides. Examples of additional peptides may include epitope peptides for labelling, such as a polyhistidine tag (His-tag), Myc, and FLAG. In some embodiments, a nuclease described herein can be fused to a detectable moiety such as a fluorescent protein (e.g., green fluorescent protein (GFP) or yellow fluorescent protein (YFP)).

A nuclease described herein can be modified to have diminished nuclease activity, e.g., nuclease inactivation of at least 50%, at least 60%, at least 70%, at least 80%, at least 90%, at least 95%, at least 97%, or 100%, as compared to a reference nuclease. Nuclease activity can be diminished by several methods known in the art, e.g., introducing mutations into the RuvC domain (e.g, one or more catalytic residues of the RuvC domain).

In some embodiments, the nuclease described herein can be self-inactivating. See, Epstein et al., “Engineering a Self-Inactivating CRISPR System for AAV Vectors,” Mol. Ther., 24 (2016): S50, which is incorporated by reference in its entirety.

Nucleic acid molecules encoding the nucleases described herein can further be codon-optimized. The nucleic acid can be codon-optimized for use in a particular host cell, such as a bacterial cell or a mammalian cell.

Described herein are gene editing systems and methods relating to a nuclease of the present invention. The gene editing systems and methods are based, in part, on the observation that cloned and expressed polypeptides of the present invention have nuclease activity. In some embodiments, the nuclease described herein is analyzed using one or more assays. In some embodiments, the biochemical characteristics of a nuclease of the present invention are analyzed in bacterial cells, as described in Example 1. In some embodiments, the biochemical characteristics of a nuclease of the present invention are analyzed in mammalian cells, as described in Example 2 and Example 3.

In some embodiments, a nuclease of the present invention has enzymatic activity, e.g., nuclease activity, over a broad range of pH conditions. In some embodiments, the nuclease has enzymatic activity, e.g., nuclease activity, at a pH of from about 3.0 to about 12.0. In some embodiments, the nuclease has enzymatic activity at a pH of from about 4.0 to about 10.5. In some embodiments, the nuclease has enzymatic activity at a pH of from about 5.5 to about 8.5. In some embodiments, the nuclease has enzymatic activity at a pH of from about 6.0 to about 8.0. In some embodiments, the nuclease has enzymatic activity at a pH of about 7.0.

In some embodiments, a nuclease of the present invention has enzymatic activity, e.g., nuclease activity, at a temperature range of from about 10° C. to about 100° C. In some embodiments, a nuclease of the present invention has enzymatic activity at a temperature range from about 20° C. to about 90° C. In some embodiments, a nuclease of the present invention has enzymatic activity at a temperature of about 20° C. to about 25° C. or at a temperature of about 37° C.

In some embodiments wherein a nuclease of the present invention induces double-stranded breaks or single-stranded breaks in a target nucleic acid, (e.g. genomic DNA), the double-stranded break can stimulate cellular endogenous DNA-repair pathways, including Homology Directed Recombination (HDR), Non-Homologous End Joining (NHEJ), or Alternative Non-Homologues End-Joining (A-NHEJ). NHEJ can repair cleaved target nucleic acid without the need for a homologous template. This can result in deletion or insertion of one or more nucleotides at the target locus. HDR can occur with a homologous template, such as the donor DNA. The homologous template can comprise sequences that are homologous to sequences flanking the target nucleic acid cleavage site. In some cases, HDR can insert an exogenous polynucleotide sequence into the cleave target locus. The modifications of the target DNA due to NHEJ and/or HDR can lead to, for example, mutations, deletions, alterations, integrations, gene correction, gene replacement, gene tagging, transgene knock-in, gene disruption, and/or gene knock-outs.

In some embodiments, binding of a nuclease/RNA guide complex to a target locus in a cell recruits one or more endogenous cellular molecules or pathways other than DNA repair pathways to modify the target nucleic acid. In some embodiments, binding of a nuclease/RNA guide complex blocks access of one or more endogenous cellular molecules or pathways to the target nucleic acid, thereby modifying the target nucleic acid. For example, binding of a nuclease/RNA guide complex may block endogenous transcription or translation machinery to decrease the expression of the target nucleic acid.

B. RNA Guide

In some embodiments, the gene editing system described herein comprises an RNA guide.

The RNA guide may be substantially identical to a reference nucleic acid sequence if the RNA guide comprises a sequence having least about 60%, least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to the reference nucleic acid sequence. The percent identity between two such nucleic acids can be determined manually by inspection of the two optimally aligned nucleic acid sequences or by using software programs or algorithms (e.g., BLAST, ALIGN, CLUSTAL) using standard parameters. One indication that two nucleic acid sequences are substantially identical is that the two nucleic acid molecules hybridize to each other under stringent conditions (e.g., within a range of medium to high stringency).

In some embodiments, the RNA guide has at least about 60%, least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to the reference nucleic acid sequence.

In some embodiments, the RNA guide sequence directs a nuclease described herein to a particular nucleic acid sequence. Those skilled in the art reading the below examples of particular kinds of RNA guide sequences will understand that, in some embodiments, an RNA guide sequence is site-specific. That is, in some embodiments, an RNA guide sequence associates specifically with one or more target nucleic acid sequences (e.g., specific DNA or genomic DNA sequences) and not to non-targeted nucleic acid sequences (e.g., non-specific DNA or random sequences).

In some embodiments, the gene editing system as described herein comprises an RNA guide sequence that associates with a nuclease described herein and directs a nuclease to a target nucleic acid sequence (e.g., DNA). The RNA guide sequence may associate with a nucleic acid sequence and alter functionality of a nuclease (e.g., alters affinity of the nuclease to a molecule, e.g., at least 10%, 15%, 20%, 25%, 30%, 35%, 40%, 45%, 50%, 55%, 60%, 65%, 70%, 75%, 80%, 85%, 90%, 95%, or more).

The RNA guide sequence may target (e.g., associate with, be directed to, contact, or bind) one or more nucleotides of a sequence, e.g., a site-specific sequence or a site-specific target. In some embodiments, a nuclease (e.g., a nuclease plus an RNA guide) is activated upon binding to a nucleic acid substrate that is complementary to a spacer sequence in the RNA guide (e.g., a sequence-specific substrate or target nucleic acid).

In some embodiments, an RNA guide sequence comprises a spacer sequence. In some embodiments, the spacer sequence of the RNA guide sequence may be generally designed to have a length of between 15 and 50 nucleotides and be complementary to a specific nucleic acid sequence. In some embodiments, the spacer is about 15-20 nucleotides in length, about 20-25 nucleotides in length, about 25-30 nucleotides in length, about 30-35 nucleotides in length, about 35-40 nucleotides in length, about 40-45 nucleotides in length, or about 45-50 nucleotides in length. In some particular embodiments, the RNA guide sequence may be designed to be complementary to a specific DNA strand, e.g., of a genomic locus. In some embodiments, the spacer sequence is designed to be complementary to a specific DNA strand, e.g., of a genomic locus.

In certain embodiments, the RNA guide sequence comprises a direct repeat sequence linked to a sequence or spacer sequence. In some embodiments, the RNA guide sequence includes a direct repeat sequence and a spacer sequence or a direct repeat-spacer-direct repeat sequence. In some embodiments, the RNA guide sequence includes a truncated direct repeat sequence and a spacer sequence, which is typical of processed or mature crRNA. In some embodiments, a nuclease forms a complex with the RNA guide sequence, and the RNA guide sequence directs the complex to associate with site-specific target nucleic acid that is complementary to at least a portion of the RNA guide sequence.

In some embodiments, the RNA guide sequence comprises a sequence, e.g., RNA sequence, at least 80%, at least 90%, at least 95%, at least 96%, at least 97%, at least 98%, at least 99% complementary to a target nucleic acid sequence. In some embodiments, the RNA guide sequence comprises a sequence at least 80%, at least 90%, at least 95%, at least 96%, at least 97%, at least 98%, at least 99% complementary to a DNA sequence. In some embodiments, the RNA guide sequence comprises a sequence at least 80%, at least 90%, at least 95%, at least 96%, at least 97%, at least 98%, at least 99% complementary to a target nucleic acid sequence. In some embodiments, the RNA guide sequence comprises a sequence at least 80%, at least 90%, at least 95%, at least 96%, at least 97%, at least 98%, at least 99% complementary to a genomic sequence. In some embodiments, the RNA guide sequence comprises a sequence complementary to or a sequence comprising at least 80%, at least 90%, at least 95%, at least 96%, at least 97%, at least 98%, at least 99% complementarity to a genomic sequence.

In some embodiments, the RNA guide binds to a first strand of the target (i.e., the non-PAM strand) and a PAM sequence as described herein is present in the second, complementary strand (i.e., the PAM strand) adjacent to the target sequence. In some embodiments, the PAM comprises a nucleotide sequence set forth in Table 5.

In some embodiments, a nuclease described herein includes one or more (e.g., two, three, four, five, six, seven, eight, or more) RNA guide sequences, e.g., RNA guides.

In some embodiments, the RNA guide has an architecture similar to, for example International Publication Nos. WO 2014/093622 and WO 2015/070083, the entire contents of each of which are incorporated herein by reference.

In some embodiments, an RNA guide sequence of the present invention comprises a direct repeat sequence having 80, 85, 90, 91, 92, 93, 94, 95, 96, 97, 98, 99 or 100% identity the direct repeat sequences of Table 2. In some embodiments, an RNA guide of the present invention comprises a direct repeat sequence having greater than 80, 85, 90, 91, 92, 93, 94, 95, 96, 97, 98, 99 or 100% identity to the direct repeat sequences of Table 2.

TABLE 2
Direct repeat sequences.

| Nuclease | Full- | |
|---|---|---|
| polypeptide | Length Direct Repeat Sequence | Mature Direct Repeat Sequence |
| SEQ ID NO: 1 | GGUGCAAUCGCCCGGAUUUCGCGACCUG | AUUUCGCGACCUGCUUACAGG (SEQ ID |
| | CUUACAGG (SEQ ID NO: 65) | NO: 66) |
| SEQ ID NO: 2 | GUCGCAUCUUGCGUAAGCGCGUGGAUUG | CUUGCGUAAGCGCGUGGAUUGAAAC (SEQ |
| | AAAC (SEQ ID NO: 67) | ID NO: 68) |
| SEQ ID NO: 3 | GCCGCAGAGCUCGGAAAGUCAGAGAAGG | AGGUAUGGCGG (SEQ ID NO: 70) |
| | UAUGGCGG (SEQ ID NO: 69) | |
| SEQ ID NO: 4 | GCGCCACUCGCUGAUGCUUGUGUUACUG | UUACUGAAUGGCGG (SEQ ID NO: 72) |
| | AAUGGCGG (SEQ ID NO: 71) | |
| SEQ ID NO: 5 | GCUGUAGAAGGGCGUCCAUUCACGGCUG | GCUGUAGAAGGGCGUCCAUUCACGGCUGACG |
| | ACGGAAAC (SEQ ID NO: 73) | GAAAC (SEQ ID NO: 74) |
| SEQ ID NO: 6 | GUUGCAACGGCUUCCGGAGUGCUGGUGG | GUUGCAACGGCUUCCGGAGUGCUGGUGGGAA |
| | GAAUGAAAC (SEQ ID NO: 75) | UGAAAC (SEQ ID NO: 76) |
| SEQ ID NO: 7 | GCUGUAGAGGGGCGUCCAUUCACGGCUG | GCUGUAGAGGGGCGUCCAUUCACGGCUGACG |
| | ACGGAAAC (SEQ ID NO: 77) | GAAAC (SEQ ID NO: 78) |
| SEQ ID NO: 8 | UGUAUAAGGGCGUCCAUUCACGGCUGAC | UGUAUAAGGGCGUCCAUUCACGGCUGACGGA |
| | GGAAAC (SEQ ID NO: 79) | AAC (SEQ ID NO: 80) |
| SEQ ID NO: 9 | GCUGUAGAGGGGCGUCCAUUCACGGCUG | GCUGUAGAGGGGCGUCCAUUCACGGCUGACG |
| | ACGGAAAC (SEQ ID NO: 81) | GAAAC (SEQ ID NO: 82) |
| SEQ ID NO: 10 | GCUGUAGAGGGGCGUCCAUUCACGGCUG | CUGACGGAAAC (SEQ ID NO: 84) |
| | ACGGAAAC (SEQ ID NO: 83) | |
| SEQ ID NO: 11 | UGUAGAAGGGCGUCCAUUCACGGCUGAC | UGUAGAAGGGCGUCCAUUCACGGCUGACGGA |
| | GGAAAC (SEQ ID NO: 85) | AAC (SEQ ID NO: 86) |
| SEQ ID NO: 12 | CCUCAUCAAUCCUAUCAAUAAUGAG | CCUCAUCAAUCCUAUCAAUAAUGAG (S EQ |
| | (SEQ ID NO: 87) | ID NO: 88) |
| SEQ ID NO: 13 | CCUUCAAAACCCUGUCACAUCUGGA | CCUUCAAAACCCUGUCACAUCUGGA (SEQ |
| | (SEQ ID NO: 89) | ID NO: 90) |
| SEQ ID NO: 14 | CCUUAUAAACCCUUCCAAUAAUGGG | CCUUAUAAACCCUUCCAAUAAUGGG (SEQ |
| | (SEQ ID NO: 91) | ID NO: 92) |
| SEQ ID NO: 15 | CUUAGAAUCUUAUGACGAACUGAGG | AUCUUAUGACGAACUGAGG (SEQ ID NO: |
| | (SEQ ID NO: 93) | 94) |
| SEQ ID NO: 16 | GUUCACGGUUACGUAGGUGAUAUGGAAG | GUUCACGGUUACGUAGGUGAUAUGGAAG |
| | (SEQ ID NO: 95) | (SEQ ID NO: 96) |
| SEQ ID NO: 17 | GGUUUGGUCACCGGCGAUUUGUGGGGUG | UGUGGGGUGACUGUGACA (SEQ ID NO: |
| | ACUGUGACA (SEQ ID NO: 97) | 98) |
| SEQ ID NO: 18 | CUCUCCACGCGCGCGCGGGAUGCGGG | CUCUCCACGCGCGCGCGGGAUGCGGG (SEQ |
| | (SEQ ID NO: 99) | ID NO: 100) |
| SEQ ID NO: 19 | GUUCACCCCACAGGCGCGUGGAGUGAUG | GUUCACCCCACAGGCGCGUGGAGUGAUGG |
| | G (SEQ ID NO: 101) | (SEQ ID NO: 102) |
| SEQ ID NO: 20 | GUUCACCCCACAGGCGCGUGGAGUGAUG | GUUCACCCCACAGGCGCGUGGAGUGAUGG |
| | G (SEQ ID NO: 103) | (SEQ ID NO: 104) |
| SEQ ID NO: 21 | GUUCACCCCACGGGUGCGUGGAGUGAUG | GUUCACCCCACGGGUGCGUGGAGUGAUGG |
| | G (SEQ ID NO: 105) | (SEQ ID NO: 106) |
| SEQ ID NO: 22 | GUCGCAACGGAUGAAUGGAUGCUGAUUG | AUGAAUGGAUGCUGAUUGAUGGAAGG (SEQ |
| | AUGGAAGG (SEQ ID NO: 107) | ID NO: 108) |
| SEQ ID NO: 23 | GUCGCAACGGAUGAAUGGAUGCUGAUUG | AUGAAUGGAUGCUGAUUGAUGGAAGG (SEQ |
| | AUGGAAGG (SEQ ID NO: 109) | ID NO: 110) |
| SEQ ID NO: 24 | UAUGGUAGAGGUGCCACCGGUUUACAUG | UAUGGUAGAGGUGCCACCGGUUUACAUGGCG |
| | GCGCCGAUACC (SEQ ID NO: 111) | CCGAUACC (SEQ ID NO: 112) |
| SEQ ID NO: 25 | UGGAACGGCCUCCUCAAAGCCAACCGAG | UGGAACGGCCUCCUCAAAGCCAACCGAGGGG |
| | GGGGUAGGCUAC (SEQ ID NO: | GUAGGCUAC (SEQ ID NO: 114) |
| | 113) | |
| SEQ ID NO: 26 | UAACAAUCUGCGGAUGGAUGUGAACUGC | AUCUGCGGAUGGAUGUGAACUGCAAG (SEQ |
| | AAG (SEQ ID NO: 115) | ID NO: 116) |
| SEQ ID NO: 27 | GUAACAAUCUGCGGAUGGAUGUGAACUG | CUGCGGAUGGAUGUGAACUGCAAG (SEQ |
| | CAAG (SEQ ID NO: 117) | ID NO: 118) |
| SEQ ID NO: 28 | GUCACAACCUAUGUGUGGUUAUGAACUG | UAUGUGUGGUUAUGAACUGCAAG (SEQ ID |
| | CAAG (SEQ ID NO: 119) | NO: 120) |
| SEQ ID NO: 29 | GGUAACAAUCUGCGGAUGGAUGUGAACU | CUGCGGAUGGAUGUGAACUGCAAG (SEQ |
| | GCAAG (SEQ ID NO: 121) | ID NO: 122) |
| SEQ ID NO: 30 | CUUGCAACUGGGCUUGGGGACUGAGGAU | CUUGCAACUGGGCUUGGGGACUGAGGAUAGU |
| | AGUUGAAAC (SEQ ID NO: 123) | UGAAAC (SEQ ID NO: 124) |
| SEQ ID NO: 31 | GCCUCAGGGGGAUAUAAGACACUCUAAA | AAAGGAAUGAAAG (SEQ ID NO: 126) |
| | GGAAUGAAAG (SEQ ID NO: 125) | |
| SEQ ID NO: 32 | GUCGCAGGGGAUCAAGAACGCUCUUAGG | ACGCUCUUAGGGAAUGAAAG (SEQ ID |
| | GAAUGAAAG (SEQ ID NO: 127) | NO: 128) |

In some embodiments, a nuclease and an RNA guide (e.g., an RNA guide comprising a direct repeat and a spacer) form a complex. In some embodiments, a nuclease and an RNA guide (e.g., an RNA guide comprising direct repeat-spacer-direct repeat sequence or pre-crRNA) form a complex. In some embodiments, the complex binds a target nucleic acid.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 1, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 65 or SEQ ID NO: 66 or a portion of the nucleotide sequence of SEQ ID NO: 65 or SEQ ID NO: 66.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 2, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 65 or SEQ ID NO: 66 or a portion of the nucleotide sequence of SEQ ID NO: 67 or SEQ ID NO: 68.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 3, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 65 or SEQ ID NO: 66 or a portion of the nucleotide sequence of SEQ ID NO: 69 or SEQ ID NO: 70.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 4, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 65 or SEQ ID NO: 66 or a portion of the nucleotide sequence of SEQ ID NO: 71 or SEQ ID NO: 72.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 5, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 65 or SEQ ID NO: 66 or a portion of the nucleotide sequence of SEQ ID NO: 73 or SEQ ID NO: 74.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 6, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 65 or SEQ ID NO: 66 or a portion of the nucleotide sequence of SEQ ID NO: 75 or SEQ ID NO: 76.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 7, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 65 or SEQ ID NO: 66 or a portion of the nucleotide sequence of SEQ ID NO: 77 or SEQ ID NO: 78.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 8, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 65 or SEQ ID NO: 66 or a portion of the nucleotide sequence of SEQ ID NO: 79 or SEQ ID NO: 80.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 9, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 65 or SEQ ID NO: 66 or a portion of the nucleotide sequence of SEQ ID NO: 81 or SEQ ID NO: 82.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 10, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 65 or SEQ ID NO: 66 or a portion of the nucleotide sequence of SEQ ID NO: 83 or SEQ ID NO: 84.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 11, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 65 or SEQ ID NO: 66 or a portion of the nucleotide sequence of SEQ ID NO: 85 or SEQ ID NO: 86.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 12, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 65 or SEQ ID NO: 66 or a portion of the nucleotide sequence of SEQ ID NO: 87 or SEQ ID NO: 88.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 13, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 65 or SEQ ID NO: 66 or a portion of the nucleotide sequence of SEQ ID NO: 89 or SEQ ID NO: 90.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 14, and the direct repeat sequence sequence of SEQ ID NO: 65 or SEQ ID NO: 66 or a portion of the nucleotide sequence of SEQ ID NO: 91 or SEQ ID NO: 92.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 15, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 65 or SEQ ID NO: 66 or a portion of the nucleotide sequence of SEQ ID NO: 93 or SEQ ID NO: 94.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 16, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 65 or SEQ ID NO: 66 or a portion of the nucleotide sequence of SEQ ID NO: 95 or SEQ ID NO: 96.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 17, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 65 or SEQ ID NO: 66 or a portion of the nucleotide sequence of SEQ ID NO: 97 or SEQ ID NO: 98.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 18, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 65 or SEQ ID NO: 66 or a portion of the nucleotide sequence of SEQ ID NO: 99 or SEQ ID NO: 100.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 19, and the direct repeat sequence 89%, 90%, 91%, 92%, 93%, 94%, 95%, 96%, 97%, 98%, 99% or 100%) identical to the nucleotide sequence of SEQ ID NO: 65 or SEQ ID NO: 66 or a portion of the nucleotide sequence of SEQ ID NO: 101 or SEQ ID NO: 102.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 20, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 65 or SEQ ID NO: 66 or a portion of the nucleotide sequence of SEQ ID NO: 103 or SEQ ID NO: 104.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 21, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 65 or SEQ ID NO: 66 or a portion of the nucleotide sequence of SEQ ID NO: 105 or SEQ ID NO: 106.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 22, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 65 or SEQ ID NO: 66 or a portion of the nucleotide sequence of SEQ ID NO: 107 or SEQ ID NO: 108.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 23, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 65 or SEQ ID NO: 66 or a portion of the nucleotide sequence of SEQ ID NO: 109 or SEQ ID NO: 110.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 24, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 65 or SEQ ID NO: 66 or a portion of the nucleotide sequence of SEQ ID NO: 111 or SEQ ID NO: 112.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 25, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 65 or SEQ ID NO: 66 or a portion of the nucleotide sequence of SEQ ID NO: 113 or SEQ ID NO: 114.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 26, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 65 or SEQ ID NO: 66 or a portion of the nucleotide sequence of SEQ ID NO: 115 or SEQ ID NO: 116.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 27, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 65 or SEQ ID NO: 66 or a portion of the nucleotide sequence of SEQ ID NO: 117 or SEQ ID NO: 118.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 28, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 65 or SEQ ID NO: 66 or a portion of the nucleotide sequence of SEQ ID NO: 119 or SEQ ID NO: 120.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 29, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 65 or SEQ ID NO: 66 or a portion of the nucleotide sequence of SEQ ID NO: 121 or SEQ ID NO: 122.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 30, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 65 or SEQ ID NO: 66 or a portion of the nucleotide sequence of SEQ ID NO: 123 or SEQ ID NO: 124.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 31, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 65 or SEQ ID NO: 66 or a portion of the nucleotide sequence of SEQ ID NO: 125 or SEQ ID NO: 126.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 32, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 65 or SEQ ID NO: 66 or a portion of the nucleotide sequence of SEQ ID NO: 127 or SEQ ID NO: 128.

In some embodiments, the RNA guide further comprises a trans-activating RNA (tracrRNA). TracrRNAs are set forth in Table 3. In some embodiments, the RNA guide forms a complex (e.g., a duplex) with the tracrRNA. In some embodiments, an RNA guide is fused to a tracrRNA. The term single-guide RNA (sgRNA) is used herein to refer to an RNA guide-tracrRNA fusion. sgRNA sequences are set forth in Table 4. In some embodiments, the RNA guide-tracrRNA duplex or sgRNA binds to a nuclease.

TABLE 3
TracrRNA sequences.

| Nuclease | |
|---|---|
| polypeptide | TracrRNAs |
| SEQ ID NO: 1 | UUUCGUUCGAGGCCGGGAGCAACGGACCGCUAGCCCAAGGGCAACCGCGGCCGCCCGGUCG |
| | AAGGCCGACCUGUACGGCCUGAAGGUUGAGAAGGCACGAAUAAGCGGAAAAACUCGUUUCC |
| | CUUCGUGUUCGCUCACCGAGCAGACGUCGCUU (SEQ ID NO: 129) |
| SEQ ID NO: 2 | GAGAGGGGUAUUCUGUUGAUGGGCGAAUCGGUAAAAGCAAUAAAAUUAAAGAUACUGGAUA |
| | UGUUUUUAGACCCCGAAUGCACAAAGCAGGAUGAUAACUGGCGCAAAGAUUUGUCUACUAU |
| | GUCCAGAUUCUGCGCUGAAGC (SEQ ID NO: 130) |
| | GAGAGGGGUAUUCUGUUGAUGGGCGAAUCGGUAAAAGCAAUAAAAUUAAAGAUACUGGAUA |
| | UGUUUUUAGACCCCGAAUGCACAAAGCAGGAUGAUAACUGGCGCAAAGAUUUGUCUACUAU |
| | GUCCAGAUUCUGCGCUGAAGC (SEQ ID NO: 131) |
| | GAGAGGGGUAUUCUGUUGAUGGGCGAAUCGGUAAAAGCAAUAAAAUUAAAGAUACUGGAUA |
| | UGUUUUUAGACCCCGAAUGCACAAAGCAGGAUGAUAACUGGCGCAAAGAUUUGUCUACUAU |
| | GUCCAGAUUCUGCGCUGAAGCGGGGAAUAUGUGUCUGCGCGACCUG (SEQ ID NO: |
| | 132) |
| | GAGAGGGGUAUUCUGUUGAUGGGCGAAUCGGUAAAAGCAAUAAAAUUAAAGAUACUGGAUA |
| | UGUUUUUAGACCCCGAAUGCACAAAGCAGGAUGAUAACUGGCGCAAAGAUUUGUCUACUAU |
| | GUCCAGAUUCUGCGCUGAAGCGGGGAAUAUGUGUCUGCGCGACCUG (SEQ ID NO: |
| | 133) |
| | UUUACUCUGUUUCGCGCGCCAGGGCAGUUAGGUGCCCUAAAAGAGCGAAGUGGCCGAAAGG |
| | AAAGGCUAACGCUUCUCUAACGCUACGGCGACCUUGGCGAAAUGCCAUCAAUACCACGCGG |
| | CCCGAAAGGGUUCGCGCGAAACUGAGU (SEQ ID NO: 134) |
| | UUUACUCUGUUUCGCGCGCCAGGGCAGUUAGGUGCCCUAAAAGAGCGAAGUGGCCGAAAGG |
| | AAAGGCUAACGCUUCUCUAACGCUACGGCGACCUUGGCGAAAUGCCAUCAAUACCACGCGG |
| | CCCGAAAGGGUUCGCGCGAAACUGAGU (SEQ ID NO: 135) |
| | UUUACUCUGUUUCGCGCGCCAGGGCAGUUAGGUGCCCUAAAAGAGCGAAGUGGCCGAAAGG |
| | AAAGGCUAACGCUUCUCUAACGCUACGGCGACCUUGGCGAAAUGCCAUCAAUACCACGCG |
| | (SEQ ID NO: 136) |
| | UUUACUCUGUUUCGCGCGCCAGGGCAGUUAGGUGCCCUAAAAGAGCGAAGUGGCCGAAAGG |
| | AAAGGCUAACGCUUCUCUAACGCUACGGCGACCUUGGCGAAAUGCCAUCAAUACCACGCG |
| | (SEQ ID NO: 137) |
| SEQ ID NO: 3 | GUCGCUAUCAGCGGCAAUGCAAUUUCCUCCGCCUUUGGAAAUUUACGUUCUAUGACUUUG |
| | (SEQ ID NO: 138) |
| | GUCGCUAUCAGCGGCAAUGCAAUUUCCUCCGCCUUUGGAAAUUUACGUUCUAUGACUUUG |
| | (SEQ ID NO: 139) |
| | GUCGCUAUCAGCGGCAAUGCAAUUUCCUCCGCCUUUGGAAAUUUACGUUCUAUGACUUUGC |
| | GAGAGCUGCGGUGCCCCGUUGCGGCCGGG (SEQ ID NO: 140) |
| | GUCGCUAUCAGCGGCAAUGCAAUUUCCUCCGCCUUUGGAAAUUUACGUUCUAUGACUUUGC |
| | GAGAGCUGCGGUGCCCCGUUGCGGCCGGG (SEQ ID NO: 141) |
| | AUACGUCGCGACAUUCCAGCAUGUAACCCGUCGGGAGGAUUGCCCCAUGAACCGUAUUUAC |
| | CAAGGUCGGAUUACCGGCAUUCUUGAUUCCAAGGAGGACGAGCGGGGCCACCCUCCCCCAC |
| | C (SEQ ID NO: 142) |
| | AUACGUCGCGACAUUCCAGCAUGUAACCCGUCGGGAGGAUUGCCCCAUGAACCGUAUUUAC |
| | CAAGGUCGGAUUACCGGCAUUCUUGAUUCCAAGGAGGACGAGCGGGGCCACCCUCCCCCAC |
| | C (SEQ ID NO: 143) |
| | AUACGUCGCGACAUUCCAGCAUGUAACCCGUCGGGAGGAUUGCCCCAUGAACCGUAUUUAC |
| | CAAGGUCGGAUUACCGGCAUUCUUGAUUCCAAGGAGGACGAGCGGGGCCACCCUCCCCCAC |
| | CCGAUCCGAAACACAAUCCAUUCUGGCGGCA (SEQ ID NO: 144) |
| | AUACGUCGCGACAUUCCAGCAUGUAACCCGUCGGGAGGAUUGCCCCAUGAACCGUAUUUAC |
| | CAAGGUCGGAUUACCGGCAUUCUUGAUUCCAAGGAGGACGAGCGGGGCCACCCUCCCCCAC |
| | CCGAUCCGAAACACAAUCCAUUCUGGCGGCA (SEQ ID NO: 145) |
| | CUUGACAUGGCUUAAGAAUGCGCCAUGUAUGAAGGAGAAGUAACCUGCUUCCUUCCUCGCU |
| | GUCACCGCCUUCGUCAGCGAUUUCAUCGGCGGAACAUAAUAAUUUUACUUUUUUCU (SEQ |
| | ID NO: 146) |
| | CUUGACAUGGCUUAAGAAUGCGCCAUGUAUGAAGGAGAAGUAACCUGCUUCCUUCCUCGCU |
| | GUCACCGCCUUCGUCAGCGAUUUCAUCGGCGGAACAUAAUAAUUUUACUUUUUUCU (SEQ |
| | ID NO: 147) |
| SEQ ID NO: 4 | GGUAUAUCCGUUUUGUUCUGCUCUCUAUCGCAUUCGGGGGGCAAUGCGUCAAAGAUUAGUG |
| | ACGUGUUUGCGAAAUAGACGUCUAUUGAUA (SEQ ID NO: 148) |
| | GGUAUAUCCGUUUUGUUCUGCUCUCUAUCGCAUUCGGGGGGCAAUGCGUCAAAGAUUAGUG |
| | ACGUGUUUGCGAAAUAGACGUCUAUUGAUA (SEQ ID NO: 149) |
| SEQ ID NO: 5 | AAAGUGUACGUCUUUUUGUACAACCUCUUCGGCCUUCUGGCUACGGGUGCCCUGGAUCACG |
| | CC (SEQ ID NO: 150) |
| | AAAGUGUACGUCUUUUUGUACAACCUCUUCGGCCUUCUGGCUACGGGUGCCCUGGAUCACG |
| | CC (SEQ ID NO: 151) |
| | AAAGUGUACGUCUUUUUGUACAACCUCUUCGGCCUUCUGGCUACGGGUGCCCUGGAUCACG |
| | CCUUUGGGACCUGGGAAACCACAACCUUUCCC (SEQ ID NO: 152) |
| | AAAGUGUACGUCUUUUUGUACAACCUCUUCGGCCUUCUGGCUACGGGUGCCCUGGAUCACG |
| | CCUUUGGGACCUGGGAAACCACAACCUUUCCC (SEQ ID NO: 153) |
| | AAAUCUCGGUCGAUGGAAAUGUUGCCCUCUUUGGUGAUGUGAUA (SEQ ID NO: 154) |
| | AAAUCUCGGUCGAUGGAAAUGUUGCCCUCUUUGGUGAUGUGAUA (SEQ ID NO: 155) |
| | CGAUUCCACCUAACUGUCUGACAGGAAACUGCUUCGCCGCAUUGAUGCUUCGA (SEQ ID |
| | NO: 156) |
| | CGAUUCCACCUAACUGUCUGACAGGAAACUGCUUCGCCGCAUUGAUGCUUCGA (SEQ ID |
| | NO: 157) |
| | AAAUUAUCGGCCGGGACGGCUCCUUUGCCCCAGGGCAGGUGAGCCCGUAGUUUCGCG |
| | (SEQ ID NO: 158) |
| | AAAUUAUCGGCCGGGACGGCUCCUUUGCCCCAGGGCAGGUGAGCCCGUAGUUUCGCG |
| | (SEQ ID NO: 159) |
| | AAAUUAUCGGCCGGGACGGCUCCUUUGCCCCAGGGCAGGUGAGCCCGUAGUUUCGCGGCCG |
| | GGUACUGGACGGAUAGGCGAAUGACG (SEQ ID NO: 160) |
| | AAAUUAUCGGCCGGGACGGCUCCUUUGCCCCAGGGCAGGUGAGCCCGUAGUUUCGCGGCCG |
| | GGUACUGGACGGAUAGGCGAAUGACG (SEQ ID NO: 161) |
| | GUUCGAUCUCUUUCAACAAUGGUUGAAGGAAAUUAUCGGCCGGGACGGCUCCUUUGCCCCA |
| | GGGCAGGUGAGCCCGUAGUUUCGCGGCCGGGUACUGGACGG (SEQ ID NO: 162) |
| | GUUCGAUCUCUUUCAACAAUGGUUGAAGGAAAUUAUCGGCCGGGACGGCUCCUUUGCCCCA |
| | GGGCAGGUGAGCCCGUAGUUUCGCGGCCGGGUACUGGACGG (SEQ ID NO: 163) |
| SEQ ID NO: 6 | GUUCCCAGCCUUCCAGGAAUGGUUGGUCGGGAUCAGACGCCGGGUUGGUGAGCCAGCUU |
| | (SEQ ID NO: 164) |
| | GUUCCCAGCCUUCCAGGAAUGGUUGGUCGGGAUCAGACGCCGGGUUGGUGAGCCAGCUU |
| | (SEQ ID NO: 165) |
| | GUUCCCAGCCUUCCAGGAAUGGUUGGUCGGGAUCAGACGCCGGGUUGGUGAGCCAGCUUUA |
| | AUCGGCCGGUGAGCCAGGUAGUUUCAUC (SEQ ID NO: 166) |
| | GUUCCCAGCCUUCCAGGAAUGGUUGGUCGGGAUCAGACGCCGGGUUGGUGAGCCAGCUUUA |
| | AUCGGCCGGUGAGCCAGGUAGUUUCAUC (SEQ ID NO: 167) |
| | AGCUUAGGCUGAAGGGAGGCGCUGCAUUCUGAGUCGGGUCCGGAGCUGUUGCAACGGCUUC |
| | CGGAGUGCUGGUGGGAAUGAAACAUGAUCUCUUUUCUCC (SEQ ID NO: 168) |
| | AGCUUAGGCUGAAGGGAGGCGCUGCAUUCUGAGUCGGGUCCGGAGCUGUUGCAACGGCUUC |
| | CGGAGUGCUGGUGGGAAUGAAACAUGAUCUCUUUUCUCC (SEQ ID NO: 169) |
| SEQ ID NO: 7 | GUUCGAUCUCUUUCAACAGUGGUUGAAGGAGCUUAUCGGCCGGGACGGUUCCUCUGCCUUC |
| | GGGCAAGGGAACCAGUAGUUUCGCGGCCGGGUACUGGGCGGAUAGGCGAAUGACGGCAGCC |
| | UCACGGCUGGCGC (SEQ ID NO: 170) |
| | GUUCGAUCUCUUUCAACAGUGGUUGAAGGAGCUUAUCGGCCGGGACGGUUCCUCUGCCUUC |
| | GGGCAAGGGAACCAGUAGUUUCGCGGCCGGGUACUGGGCGGAUAGGCGAAUGACGGCAGCC |
| | UCACGGCUGGCGC (SEQ ID NO: 171) |
| | GUUCGAUCUCUUUCAACAGUGGUUGAAGGAGCUUAUCGGCCGGGACGGUUCCUCUGCCUUC |
| | GGGCAAGGGAACCAGUAGUUUCGCGGCCGGGUACUGGGCGGAUAGGCGAAUGACGGCAGCC |
| | UCACGGCUGGCGCGUAACCGACCAUCCCCUGGUCCGAACAGCC (SEQ ID NO: 172) |
| | GUUCGAUCUCUUUCAACAGUGGUUGAAGGAGCUUAUCGGCCGGGACGGUUCCUCUGCCUUC |
| | GGGCAAGGGAACCAGUAGUUUCGCGGCCGGGUACUGGGCGGAUAGGCGAAUGACGGCAGCC |
| | UCACGGCUGGCGCGUAACCGACCAUCCCCUGGUCCGAACAGCC (SEQ ID NO: 173) |
| | GUUCGAUCUCUUUCAACAGUGGUUGAAGGAGCUUAUCGGCCGGGACGGUUCCUCUGCCUUC |
| | GGGCAAGGGAACCAGUAGUUUCGCGGCCGGGUACUGGGC (SEQ ID NO: 174) |
| | GUUCGAUCUCUUUCAACAGUGGUUGAAGGAGCUUAUCGGCCGGGACGGUUCCUCUGCCUUC |
| | GGGCAAGGGAACCAGUAGUUUCGCGGCCGGGUACUGGGC (SEQ ID NO: 175) |
| SEQ ID NO: 8 | GUUCGAUCUCUUUCAGCAAUGGUUGAAGGAGCUUAUCGGCCGGGACGGUUCUGUUGCCCUC |
| | GGGCAGGGGAACCAGUAGUUUCGCGGCCGGGUACUGGGC (SEQ ID NO: 176) |
| | GUUCGAUCUCUUUCAGCAAUGGUUGAAGGAGCUUAUCGGCCGGGACGGUUCUGUUGCCCUC |
| | GGGCAGGGGAACCAGUAGUUUCGCGGCCGGGUACUGGGC (SEQ ID NO: 177) |
| SEQ ID NO: 9 | GAAGUUCGAUCUCUUUCAACAGUGGUUGAAGGAGCUUAUCGGCCGGGACGGUUCCGCUGCC |
| | CCAGGGUAGUGGAACCAGUAGUUUCGCGGCCGGGUACUGGGCGGAUAGGCGAAUGACGGCA |
| | UCCUCACGGUUGCCGC (SEQ ID NO: 178) |
| | GAAGUUCGAUCUCUUUCAACAGUGGUUGAAGGAGCUUAUCGGCCGGGACGGUUCCGCUGCC |
| | CCAGGGUAGUGGAACCAGUAGUUUCGCGGCCGGGUACUGGGCGGAUAGGCGAAUGACGGCA |
| | UCCUCACGGUUGCCGC (SEQ ID NO: 179) |
| | GAAGUUCGAUCUCUUUCAACAGUGGUUGAAGGAGCUUAUCGGCCGGGACGGUUCCGCUGCC |
| | CCAGGGUAGUGGAACCAGUAGUUUCGCGGCCGGGUACUGGGCGGAUAGGCGAAUGACGGCA |
| | UCCUCACGGUUGCCGCGUAACCGACCAUCCCCUGGUCCGAACAGCC (SEQ ID NO: |
| | 180) |
| | GAAGUUCGAUCUCUUUCAACAGUGGUUGAAGGAGCUUAUCGGCCGGGACGGUUCCGCUGCC |
| | CCAGGGUAGUGGAACCAGUAGUUUCGCGGCCGGGUACUGGGCGGAUAGGCGAAUGACGGCA |
| | UCCUCACGGUUGCCGCGUAACCGACCAUCCCCUGGUCCGAACAGCC (SEQ ID NO: |
| | 181) |
| | GAAGUUCGAUCUCUUUCAACAGUGGUUGAAGGAGCUUAUCGGCCGGGACGGUUCCGCUGCC |
| | CCAGGGUAGUGGAACCAGUAGUUUCGCGGCCGGGUACUGGGC (SEQ ID NO: 182) |
| | GAAGUUCGAUCUCUUUCAACAGUGGUUGAAGGAGCUUAUCGGCCGGGACGGUUCCGCUGCC |
| | CCAGGGUAGUGGAACCAGUAGUUUCGCGGCCGGGUACUGGGC (SEQ ID NO: 183) |
| SEQ ID NO: 10 | GUUCGAUCUCUUUCAACUGUGGUUGAAGGAGCUUAUCGGCCGGGACGGUUCCAUUGCCUCA |
| | GGGCAGGGGAACCAGUAGUUUCGCGGCCGGGUACUGGGCGGAUAGGCGAAUGAUGGCAUCC |
| | UCACGGUUGCCGC (SEQ ID NO: 184) |
| | GUUCGAUCUCUUUCAACUGUGGUUGAAGGAGCUUAUCGGCCGGGACGGUUCCAUUGCCUCA |
| | GGGCAGGGGAACCAGUAGUUUCGCGGCCGGGUACUGGGCGGAUAGGCGAAUGAUGGCAUCC |
| | UCACGGUUGCCGC (SEQ ID NO: 185) |
| | GUUCGAUCUCUUUCAACUGUGGUUGAAGGAGCUUAUCGGCCGGGACGGUUCCAUUGCCUCA |
| | GGGCAGGGGAACCAGUAGUUUCGCGGCCGGGUACUGGGCGGAUAGGCGAAUGAUGGCAUCC |
| | UCACGGUUGCCGCGUAACCGACCAUCCCCUGGUCCGAACAGCC (SEQ ID NO: 186) |
| | GUUCGAUCUCUUUCAACUGUGGUUGAAGGAGCUUAUCGGCCGGGACGGUUCCAUUGCCUCA |
| | GGGCAGGGGAACCAGUAGUUUCGCGGCCGGGUACUGGGCGGAUAGGCGAAUGAUGGCAUCC |
| | UCACGGUUGCCGCGUAACCGACCAUCCCCUGGUCCGAACAGCC (SEQ ID NO: 187) |
| | AUAUCUCAAGCCAUGUUCAAAACGGCGCGUUUCAAGGUUCACAAUCCGUCACGGCACAAGA |
| | GCACUAUGCUCUGGUAUGCCAUGACCCGCUAUCACGAGACUUUGAAGGACGUACUCGAAAA |
| | GACACUGGCGAUUCCAGAUCUGCUAGAACA (SEQ ID NO: 188) |
| | AUAUCUCAAGCCAUGUUCAAAACGGCGCGUUUCAAGGUUCACAAUCCGUCACGGCACAAGA |
| | GCACUAUGCUCUGGUAUGCCAUGACCCGCUAUCACGAGACUUUGAAGGACGUACUCGAAAA |
| | GACACUGGCGAUUCCAGAUCUGCUAGAACA (SEQ ID NO: 189) |
| | AUAUCUCAAGCCAUGUUCAAAACGGCGCGUUUCAAGGUUCACAAUCCGUCACGGCACAAGA |
| | GCACUAUGCUCUGGUAUGCCAUGACCCGCUAUCACGAGACUUUGAAGGACGUACUCGAAAA |
| | GACACUGGCGAUUCCAGAUCUGCUAGAACAAAUCUCAGAA (SEQ ID NO: 190) |
| | AUAUCUCAAGCCAUGUUCAAAACGGCGCGUUUCAAGGUUCACAAUCCGUCACGGCACAAGA |
| | GCACUAUGCUCUGGUAUGCCAUGACCCGCUAUCACGAGACUUUGAAGGACGUACUCGAAAA |
| | GACACUGGCGAUUCCAGAUCUGCUAGAACAAAUCUCAGAA (SEQ ID NO: 191) |
| | GUUCGAUCUCUUUCAACUGUGGUUGAAGGAGCUUAUCGGCCGGGACGGUUCCAUUGCCUCA |
| | GGGCAGGGGAACCAGUAGUUUCGCGGCCGGGUACUGGG (SEQ ID NO: 192) |
| | GUUCGAUCUCUUUCAACUGUGGUUGAAGGAGCUUAUCGGCCGGGACGGUUCCAUUGCCUCA |
| | GGGCAGGGGAACCAGUAGUUUCGCGGCCGGGUACUGGGC (SEQ ID NO: 193) |
| SEQ ID NO: 11 | CAAAUUCGGUGCAAGGGCGAAGCCCAACCGGUAGACUCAUAUCUUCCGGUAACGCACCUCG |
| | GCUGUUUAGACC (SEQ ID NO: 194) |
| | CAAAUUCGGUGCAAGGGCGAAGCCCAACCGGUAGACUCAUAUCUUCCGGUAACGCACCUCG |
| | GCUGUUUAGACC (SEQ ID NO: 195) |
| | CAAAUUCGGUGCAAGGGCGAAGCCCAACCGGUAGACUCAUAUCUUCCGGUAACGCACCUCG |
| | GCUGUUUAGACCAGAGGAUGGUCGGUUACGCGGCAACCGUGA (SEQ ID NO: 196) |
| | CAAAUUCGGUGCAAGGGCGAAGCCCAACCGGUAGACUCAUAUCUUCCGGUAACGCACCUCG |
| | GCUGUUUAGACCAGAGGAUGGUCGGUUACGCGGCAACCGUGA (SEQ ID NO: 197) |
| | GUUCGAUAUCUUUCAACAGUGGUUGAAGGAGCUUAUCGGCCGGGACGGUUCCUCUGCCCCA |
| | GGGCAGUUGAACCCGUAGUUUCGCGGCCGGGUACUGGGC (SEQ ID NO: 198) |
| | GUUCGAUAUCUUUCAACAGUGGUUGAAGGAGCUUAUCGGCCGGGACGGUUCCUCUGCCCCA |
| | GGGCAGUUGAACCCGUAGUUUCGCGGCCGGGUACUGGGC (SEQ ID NO: 199) |
| SEQ ID NO: 12 | AUUGAUGAGGCUGUCUGACUUGUGCAGGCUCAUUAUUGAUAGGAUUGAUGAGGAUGACUUA |
| | AAACGCGAAA (SEQ ID NO: 200) |
| | AUUGAUGAGGCUGUCUGACUUGUGCAGGCUCAUUAUUGAUAGGAUUGAUGAGGAUGACUUA |
| | AAACGCGAAA (SEQ ID NO: 201) |
| | AUUGAUGAGGCUGUCUGACUUGUGCAGGCUCAUUAUUGAUAGGAUUGAUGAGGAUGACUUA |
| | AAACGCGAAACUCAUUAUUGAUAGGAUUGAUGAGGAUUUU (SEQ ID NO: 202) |
| | AUUGAUGAGGCUGUCUGACUUGUGCAGGCUCAUUAUUGAUAGGAUUGAUGAGGAUGACUUA |
| | AAACGCGAAACUCAUUAUUGAUAGGAUUGAUGAGGAUUUU (SEQ ID NO: 203) |
| | ACAAUCCCUCUUGAGCAUUGGAUGCAGAUUCUUCUGUCUCCGUACCUCGCUCAUAAGGGCG |
| | CUGUCGGGAUAGCAGUGCCUAUCAAAAC (SEQ ID NO: 204) |
| | ACAAUCCCUCUUGAGCAUUGGAUGCAGAUUCUUCUGUCUCCGUACCUCGCUCAUAAGGGCG |
| | CUGUCGGGAUAGCAGUGCCUAUCAAAAC (SEQ ID NO: 205) |
| | ACAAUCCCUCUUGAGCAUUGGAUGCAGAUUCUUCUGUCUCCGUACCUCGCUCAUAAGGGCG |
| | CUGUCGGGAUAGCAGUGCCUAUCAAAACUAUCCCACUAUAAUGCUUGUUUCUUUACCU |
| | (SEQ ID NO: 206) |
| | ACAAUCCCUCUUGAGCAUUGGAUGCAGAUUCUUCUGUCUCCGUACCUCGCUCAUAAGGGCG |
| | CUGUCGGGAUAGCAGUGCCUAUCAAAACUAUCCCACUAUAAUGCUUGUUUCUUUACCU |
| | (SEQ ID NO: 207) |
| | UAUUGAUAGGAUUGAUGAGGUCGAGCAUCGAAAAGAUU (SEQ ID NO: 208) |
| | UAUUGAUAGGAUUGAUGAGGUCGAGCAUCGAAAAGAUU (SEQ ID NO: 209) |
| | UAUUGAUAGGAUUGAUGAGGUCGAGCAUCGAAAAGAUUCUCAUUAUUGAUAGGAUUGAUGA |
| | GGCUUAA (SEQ ID NO: 210) |
| | UAUUGAUAGGAUUGAUGAGGUCGAGCAUCGAAAAGAUUCUCAUUAUUGAUAGGAUUGAUGA |
| | GGCUUAA (SEQ ID NO: 211) |
| SEQ ID NO: 13 | CUCAAACUCCUUCCGGCUUUCGGACCGUCUGCUUGCAGACCUCCUUACCUUGGCCGGAGGG |
| | ACAUCGCCGGGGGUCGAUGCCUGUCACUUACCCCCAUU (SEQ ID NO: 212) |
| | CUCAAACUCCUUCCGGCUUUCGGACCGUCUGCUUGCAGACCUCCUUACCUUGGCCGGAGGG |
| | ACAUCGCCGGGGGUCGAUGCCUGUCACUUACCCCCAUU (SEQ ID NO: 213) |
| | CUCAAACUCCUUCCGGCUUUCGGACCGUCUGCUUGCAGACCUCCUUACCUUGGCCGGAGGG |
| | ACAUCGCCGGGGGUCGAUGCCUGUCACUUACCCCCAUUUCACCCACUUCUUGGCUUGGUUU |
| | CUCCGGG (SEQ ID NO: 214) |
| | CUCAAACUCCUUCCGGCUUUCGGACCGUCUGCUUGCAGACCUCCUUACCUUGGCCGGAGGG |
| | ACAUCGCCGGGGGUCGAUGCCUGUCACUUACCCCCAUUUCACCCACUUCUUGGCUUGGUUU |
| | CUCCGGG (SEQ ID NO: 215) |
| | GCACUGACACGCCCAGAAAUGACAGGGUUCUAAUCGGCUGAUCCCGCCUUGAAUUGACCAG |
| | AGGCCGUAAGGAUUCAAAAGGACUUCAAUGCGGCCUCAUGUCUCU (SEQ ID NO: |
| | 216) |
| | GCACUGACACGCCCAGAAAUGACAGGGUUCUAAUCGGCUGAUCCCGCCUUGAAUUGACCAG |
| | AGGCCGUAAGGAUUCAAAAGGACUUCAAUGCGGCCUCAUGUCUCU (SEQ ID NO: |
| | 217) |
| | GCACUGACACGCCCAGAAAUGACAGGGUUCUAAUCGGCUGAUCCCGCCUUGAAUUGACCAG |
| | AGGCCGUAAGGAUUCAAAAGGACUUCAAUGCGGCCUCAUGUCUCUCUCCGGUUCGACCGGC |
| | UACGGGAAGGUUCU (SEQ ID NO: 218) |
| | GCACUGACACGCCCAGAAAUGACAGGGUUCUAAUCGGCUGAUCCCGCCUUGAAUUGACCAG |
| | AGGCCGUAAGGAUUCAAAAGGACUUCAAUGCGGCCUCAUGUCUCUCUCCGGUUCGACCGGC |
| | UACGGGAAGGUUCU (SEQ ID NO: 219) |
| SEQ ID NO: 14 | UCAGAAACUCCCGACUUCCUCAGGAUGAAGUUGAUUCUUUAUCCAUACCUUGGUGCCGGGA |
| | CGCCGAUUGAGGAAUGGGCGGCGCCUUCCAAAUUCUCAUUCCUCACU (SEQ ID NO: |
| | 220) |
| | UCAGAAACUCCCGACUUCCUCAGGAUGAAGUUGAUUCUUUAUCCAUACCUUGGUGCCGGGA |
| | CGCCGAUUGAGGAAUGGGCGGCGCCUUCCAAAUUCUCAUUCCUCACU (SEQ ID NO: |
| | 221) |
| | UCAGAAACUCCCGACUUCCUCAGGAUGAAGUUGAUUCUUUAUCCAUACCUUGGUGCCGGGA |
| | CGCCGAUUGAGGAAUGGGCGGCGCCUUCCAAAUUCUCAUUCCUCACUUUCCUCCUGCGAAA |
| | CUUCAUCGAAGCAAAU (SEQ ID NO: 222) |
| | UCAGAAACUCCCGACUUCCUCAGGAUGAAGUUGAUUCUUUAUCCAUACCUUGGUGCCGGGA |
| | CGCCGAUUGAGGAAUGGGCGGCGCCUUCCAAAUUCUCAUUCCUCACUUUCCUCCUGCGAAA |
| | CUUCAUCGAAGCAAAU (SEQ ID NO: 223) |
| | CAGAAACUCCCGACUUCCUCAGGAUGAAGUUGAUUCUUUAUCCAUACCUUGGUGCCGGGAC |
| | GCCGAUUGAGGAAUGGGCGGCGCCUUCCAAAUUCUCAUUCCUCACUU (SEQ ID NO: |
| | 224) |
| | CAGAAACUCCCGACUUCCUCAGGAUGAAGUUGAUUCUUUAUCCAUACCUUGGUGCCGGGAC |
| | GCCGAUUGAGGAAUGGGCGGCGCCUUCCAAAUUCUCAUUCCUCACUU (SEQ ID NO: |
| | 225) |
| | CAGAAACUCCCGACUUCCUCAGGAUGAAGUUGAUUCUUUAUCCAUACCUUGGUGCCGGGAC |
| | GCCGAUUGAGGAAUGGGCGGCGCCUUCCAAAUUCUCAUUCCUCACUUUCCUCCUGCGAAAC |
| | UUCAUCGAAGCAAAUU (SEQ ID NO: 226) |
| | CAGAAACUCCCGACUUCCUCAGGAUGAAGUUGAUUCUUUAUCCAUACCUUGGUGCCGGGAC |
| | GCCGAUUGAGGAAUGGGCGGCGCCUUCCAAAUUCUCAUUCCUCACUUUCCUCCUGCGAAAC |
| | UUCAUCGAAGCAAAUU (SEQ ID NO: 227) |
| SEQ ID NO: 15 | ACUAGUUAAAGAGGAGAAUAGAUUAUGGAACUAACAGAAGAACAGCACCAAGAUUUCAUUU |
| | GGACUUGGCCCUCGGUCCACUCCAGAGGUAAAGCCAGUCUCUGGUUACCUUAUUUUCAAGG |
| | UUUAGAGAAGUU (SEQ ID NO: 228) |
| | ACUAGUUAAAGAGGAGAAUAGAUUAUGGAACUAACAGAAGAACAGCACCAAGAUUUCAUUU |
| | GGACUUGGCCCUCGGUCCACUCCAGAGGUAAAGCCAGUCUCUGGUUACCUUAUUUUCAAGG |
| | UUUAGAGAAGUU (SEQ ID NO: 229) |
| | ACUAGUUAAAGAGGAGAAUAGAUUAUGGAACUAACAGAAGAACAGCACCAAGAUUUCAUUU |
| | GGACUUGGCCCUCGGUCCACUCCAGAGGUAAAGCCAGUCUCUGGUUACCUUAUUUUCAAGG |
| | UUUAGAGAAGUUGGGUCGCGGUAAGCCUUACCAGUAUAAGUU (SEQ ID NO: 230) |
| | ACUAGUUAAAGAGGAGAAUAGAUUAUGGAACUAACAGAAGAACAGCACCAAGAUUUCAUUU |
| | GGACUUGGCCCUCGGUCCACUCCAGAGGUAAAGCCAGUCUCUGGUUACCUUAUUUUCAAGG |
| | UUUAGAGAAGUUGGGUCGCGGUAAGCCUUACCAGUAUAAGUU (SEQ ID NO: 231) |
| | AGUUCGAAGGUACUUGUUGAUUGAUCGGGUCAAACCAAUAAUCAGCCUAGGAACAAUUUUG |
| | AAU (SEQ ID NO: 232) |
| | AGUUCGAAGGUACUUGUUGAUUGAUCGGGUCAAACCAAUAAUCAGCCUAGGAACAAUUUUG |
| | AAU (SEQ ID NO: 233) |
| SEQ ID NO: 16 | AUGGGGUUUGGUAUAUCUUCCGCUCCAUUAUCUUGGUGUAAAGUGUAUGAGCCU (SEQ |
| | ID NO: 234) |
| | AUGGGGUUUGGUAUAUCUUCCGCUCCAUUAUCUUGGUGUAAAGUGUAUGAGCCU (SEQ |
| | ID NO: 235) |
| | AUGGGGUUUGGUAUAUCUUCCGCUCCAUUAUCUUGGUGUAAAGUGUAUGAGCCUAUCUUGG |
| | CCUUGCUCUACUCUAGGUUAGAAAGUGCUCUCAACCU (SEQ ID NO: 236) |
| | AUGGGGUUUGGUAUAUCUUCCGCUCCAUUAUCUUGGUGUAAAGUGUAUGAGCCUAUCUUGG |
| | CCUUGCUCUACUCUAGGUUAGAAAGUGCUCUCAACCU (SEQ ID NO: 237) |
| SEQ ID NO: 17 | AGAAGGGACAGAAACUCAAAGACCAUGAACAGUUUUCGGCGUGGUACGCCGAUCUGAAAAA |
| | CGGGGGUGGAGGCCAUGCAGAUGGUCCUUAGUUACAGCCUCCGUGGACU (SEQ ID NO: |
| | 238) |
| | AGAAGGGACAGAAACUCAAAGACCAUGAACAGUUUUCGGCGUGGUACGCCGAUCUGAAAAA |
| | CGGGGGUGGAGGCCAUGCAGAUGGUCCUUAGUUACAGCCUCCGUGGACU (SEQ ID NO: |
| | 239) |
| | AGAAGGGACAGAAACUCAAAGACCAUGAACAGUUUUCGGCGUGGUACGCCGAUCUGAAAAA |
| | CGGGGGUGGAGGCCAUGCAGAUGGUCCUUAGUUACAGCCUCCGUGGACUGCGUUGCGGGUG |
| | UAUGACCGGAAGUUUCUU (SEQ ID NO: 240) |
| | AGAAGGGACAGAAACUCAAAGACCAUGAACAGUUUUCGGCGUGGUACGCCGAUCUGAAAAA |
| | CGGGGGUGGAGGCCAUGCAGAUGGUCCUUAGUUACAGCCUCCGUGGACUGCGUUGCGGGUG |
| | UAUGACCGGAAGUUUCUU (SEQ ID NO: 241) |
| | AGAAGGGACAGAAACUCAAAGACCAUGAACAGUUUUCGGCGUGGUACGCCGAUCUGAAAAA |
| | CGGGGGU (SEQ ID NO: 242) |
| | AGAAGGGACAGAAACUCAAAGACCAUGAACAGUUUUCGGCGUGGUACGCCGAUCUGAAAAA |
| | CGGGGGU (SEQ ID NO: 243) |
| SEQ ID NO: 18 | CUACACAGCCGGCGGGACGUAUCCCGUCGGCCGCGCCCGACUGCACGGGCGAGGCAGUGAA |
| | GGCGUCAGACGU (SEQ ID NO: 244) |
| | CUACACAGCCGGCGGGACGUAUCCCGUCGGCCGCGCCCGACUGCACGGGCGAGGCAGUGAA |
| | GGCGUCAGACGU (SEQ ID NO: 245) |
| | CUACACAGCCGGCGGGACGUAUCCCGUCGGCCGCGCCCGACUGCA (SEQ ID NO: |
| | 246) |
| | CUACACAGCCGGCGGGACGUAUCCCGUCGGCCGCGCCCGACUGCA (SEQ ID NO: |
| | 247) |
| SEQ ID NO: 19 | AUGGCAGGCGCUGCCUAGUGCGCCCGUCGCGGUAGUAAUCACGUCGAGACGCAAAACGCCU |
| | GGGGACGGUGUAGGUAGCAAACCGGAUGAACCAGGGAUU (SEQ ID NO: 248) |
| SEQ ID NO: 20 | AUGGCAGGCGCUGCCUAGUGCGCCCGUCGCGGUAGUAAUCACGUCGAGACGCAAAACGCCU |
| | GGGGACGGUGUAGGUAGCAAACCGGAUGAACCAGGA (SEQ ID NO: 249) |
| | AUGGCAGGCGCUGCCUAGUGCGCCCGUCGCGGUAGUAAUCACGUCGAGACGCAAAACGCCU |
| | GGGGACGGUGUAGGUAGCAAACCGGAUGAACCAGGA (SEQ ID NO: 250) |
| | GCAGUGCCCGACCUGCGGCGCAGAACAUGAUCG (SEQ ID NO: 251) |
| | GCAGUGCCCGACCUGCGGCGCAGAACAUGAUCG (SEQ ID NO: 252) |
| | GCAGUGCCCGACCUGCGGCGCAGAACAUGAUCGGGACGGCAACGCAGCGGUCAACAUCCGC |
| | AA (SEQ ID NO: 253) |
| | GCAGUGCCCGACCUGCGGCGCAGAACAUGAUCGGGACGGCAACGCAGCGGUCAACAUCCGC |
| | AA (SEQ ID NO: 254) |
| SEQ ID NO: 21 | AUGGCGAAACCACGAACCAAACAAGAGUACGGCCCGUUUAGCGUGCGUCUGCCUGUUGAGA |
| | UGCGCCAGCAGAUCGAAACGUUGGCAGAACAAGAGAUGCGGUCGCUUCACUCGAUG (SEQ |
| | ID NO: 255) |
| | AUGGCGAAACCACGAACCAAACAAGAGUACGGCCCGUUUAGCGUGCGUCUGCCUGUUGAGA |
| | UGCGCCAGCAGAUCGAAACGUUGGCAGAACAAGAGAUGCGGUCGCUUCACUCGAUG (SEQ |
| | ID NO: 256) |
| SEQ ID NO: 22 | GCAGCGUCGCGGGGUUUUAAUGGGCCGACUUCCCGGCCUGAAGCCGAGGUAGCAGCCAUGU |
| | ACGGCGAUUCUCGAAUCGAGGAAGGAAACAUGGACGCGCUCGGUAAAUC (SEQ ID NO: |
| | 257) |
| | GCAGCGUCGCGGGGUUUUAAUGGGCCGACUUCCCGGCCUGAAGCCGAGGUAGCAGCCAUGU |
| | ACGGCGAUUCUCGAAUCGAGGAAGGAAACAUGGACGCGCUCGGUAAAUC (SEQ ID NO: |
| | 258) |
| | UCGAGGAAGGAAACAUGGACGCGCUCGGUAAAUCGUCCCGAGAUGGGACCAAUCAGCAUCC |
| | GCCUUGCGACGCUGCC (SEQ ID NO: 259) |
| | UCGAGGAAGGAAACAUGGACGCGCUCGGUAAAUCGUCCCGAGAUGGGACCAAUCAGCAUCC |
| | GCCUUGCGACGCUGCC (SEQ ID NO: 260) |
| SEQ ID NO: 23 | AAUGGGCCGACUUCCCGGCCUGAAGCCGAGGUAGCAGCCAUGUACGGCGAUUCUCGAAUCG |
| | AGGAAGGAAACAUGGACGCGCUCGGUAAAUCGUCCCGAGAUGGGACCAAUCAGC (SEQ |
| | ID NO: 261) |
| | AAUGGGCCGACUUCCCGGCCUGAAGCCGAGGUAGCAGCCAUGUACGGCGAUUCUCGAAUCG |
| | AGGAAGGAAACAUGGACGCGCUCGGUAAAUCGUCCCGAGAUGGGACCAAUCAGC (SEQ |
| | ID NO: 262) |
| | GCAGCGUCGCGGGGUUUUAAUGGGCCGACUUCCCGGCCUGAAGCCGAGGUAGCAGCCAUGU |
| | ACGGCGAUUCUCGAAUCGAGGAAGGAAACAUGGACGCGCUCGGUAAAUCGUCCCGAGAUGG |
| | GACCAAUC (SEQ ID NO: 263) |
| | GCAGCGUCGCGGGGUUUUAAUGGGCCGACUUCCCGGCCUGAAGCCGAGGUAGCAGCCAUGU |
| | ACGGCGAUUCUCGAAUCGAGGAAGGAAACAUGGACGCGCUCGGUAAAUCGUCCCGAGAUGG |
| | GACCAAUC (SEQ ID NO: 264) |
| SEQ ID NO: 24 | GAAACCAUAGGUAGAGGCGCCACCACCUUACAUGGUGCCGAUACCGCUCCGUUGGUGCAGU |
| | GUGGACUGUAAUGGUAGAGGCU (SEQ ID NO: 265) |
| | GAAACCAUAGGUAGAGGCGCCACCACCUUACAUGGUGCCGAUACCGCUCCGUUGGUGCAGU |
| | GUGGACUGUAAUGGUAGAGGCU (SEQ ID NO: 266) |
| | UUAAUAAUCUGAUUACGGCUGAUUGCCGCCGGUAGAGGUGCCACCGCCUUACAUGACACUG |
| | AUACCUUAUAUCCAGCCGUAUU (SEQ ID NO: 267) |
| | UUAAUAAUCUGAUUACGGCUGAUUGCCGCCGGUAGAGGUGCCACCGCCUUACAUGACACUG |
| | AUACCUUAUAUCCAGCCGUAUU (SEQ ID NO: 268) |
| SEQ ID NO: 25 | AAGAAUACUGAUAUUCUCUAUCGGUCCGAAAAGUUUGAAUUGUUUUGGAAUCGUAGGCCGG |
| | UUUGUGCGCCAACCGCAGAAGAGCUGGCCUUGCUCACAAUCACCAGUGAAAACUUGCGGAC |
| | AGUAUGGAACGAA (SEQ ID NO: 269) |
| | AAGAAUACUGAUAUUCUCUAUCGGUCCGAAAAGUUUGAAUUGUUUUGGAAUCGUAGGCCGG |
| | UUUGUGCGCCAACCGCAGAAGAGCUGGCCUUGCUCACAAUCACCAGUGAAAACUUGCGGAC |
| | AGUAUGGAACGAA (SEQ ID NO: 270) |
| | UAUGCUCAUUGAAAACAAGGCAUGAGAAAAAUACCCCCGGUUUUGGGCCGGAAUGGAAUGU |
| | UUUUCUCACUGCCAUACCGUUUUUAUGAGGCAACCCUUUAGGGCACUGCAUUGGGCAGUGU |
| | UUAUGCGCUGU (SEQ ID NO: 271) |
| | UAUGCUCAUUGAAAACAAGGCAUGAGAAAAAUACCCCCGGUUUUGGGCCGGAAUGGAAUGU |
| | UUUUCUCACUGCCAUACCGUUUUUAUGAGGCAACCCUUUAGGGCACUGCAUUGGGCAGUGU |
| | UUAUGCGCUGU (SEQ ID NO: 272) |
| SEQ ID NO: 26 | CUUAUCGUGAGGUGCAGCCACGAUGUGCGAAUCUAAAGUGGACAGAAAUGGGAACAGACGU |
| | UCGCACGAAUGUUGGCGGAUUUCUUCGGAAAUCGAGCCAAUCAUCAUAUCCAUAUU (SEQ |
| | ID NO: 273) |
| SEQ ID NO: 27 | CUUAUCGUGAGGUGCAGCCACGAUGUGCGAAUCUAAAGUGAACAGAAAUGGGAACAGACGU |
| | UCGCACGAAUGUUGGCGGAUUUCUUCGGAAAUCGAGCCAAUAAUCAUAUCCAUAUUACCGG |
| | GUUG (SEQ ID NO: 274) |
| SEQ ID NO: 28 | GUGAGGUGCAGCCACGAGGUGCGAAUAGGAAGUACGCAGCAAUGUGCUGAAUCGUUCGCAC |
| | GAAAAUUGGCAUUUUUGAAAACCAAAGCCAAUAAUCAUAUCCAUAAA (SEQ ID NO: |
| | 275) |
| SEQ ID NO: 29 | CUUAUCGUGAGGUGCAGCCACGAUGUGCGAAUCUAAAGUGAACAGAAAUGGGAACAGACGU |
| | UCGCACGAAUGUUGGCGGAUUUCUUCGGAAAUCGAGCCAAUAAUCAUAUCCAUAUUACCGG |
| | GUUGCUCUAUAGAUAGUGUUGGGUACACUUGCUGAACACA (SEQ ID NO: 276) |
| | CUUAUCGUGAGGUGCAGCCACGAUGUGCGAAUCUAAAGUGAACAGAAAUGGGAACAGACGU |
| | UCGCACGAAUGUUGGCGGAUUUCUUCGGAAAUCGAGCCAAUAAUCAUAUCCAUAUUACCGG |
| | GUUGCUCUAUAGAUAGUGUUGGGUACACUUGCUGAACACA (SEQ ID NO: 277) |
| | CUUAUCGUGAGGUGCAGCCACGAUGUGCGAAUCUAAAGUGAACAGAAAUGGGAACAGACGU |
| | UCGCACGAAUGUUGGCGGAUUUCUUCGGAAAUCGAGCCAAUAAUCAUAUCCAUAUUACCGG |
| | GUUGCUCUAUAGAUAGUGUUGGGUACACUUGCUGAACACAAGAUAUUGC (SEQ ID NO: |
| | 278) |
| | CUUAUCGUGAGGUGCAGCCACGAUGUGCGAAUCUAAAGUGAACAGAAAUGGGAACAGACGU |
| | UCGCACGAAUGUUGGCGGAUUUCUUCGGAAAUCGAGCCAAUAAUCAUAUCCAUAUUACCGG |
| | GUUGCUCUAUAGAUAGUGUUGGGUACACUUGCUGAACACAAGAUAUUGC (SEQ ID NO: |
| | 279) |
| | CUUAUCGUGAGGUGCAGCCACGAUGUGCGAAUCUAAAGUGAACAGAAAUGGGAACAGACGU |
| | UCGCACGAAUGUUGGCGGAUUUCUUCGGAAAUCGAGCCAAUAAUCAUAUCCAUAUUACCGG |
| | GUUGCUCU (SEQ ID NO: 280) |
| | CUUAUCGUGAGGUGCAGCCACGAUGUGCGAAUCUAAAGUGAACAGAAAUGGGAACAGACGU |
| | UCGCACGAAUGUUGGCGGAUUUCUUCGGAAAUCGAGCCAAUAAUCAUAUCCAUAUUACCGG |
| | GUUGCUCU (SEQ ID NO: 281) |
| SEQ ID NO: 30 | UACCGCAGUUUGGGCGGGUUGCUAAUAAUAUUAGCUUAUGACUUCUAAGGCUUUUGCCUAA |
| | AAAGUAAGGGGGAAGGCGACAACCCCCAAAGACCAGCCGGAACUAUGGCUGGCAACUAUCU |
| | CAUCUUUUUGGCAUAUCAAAGCUAGGGCAAAAACCCCAGACAUUCGCCAAAAGCCCAGAAC |
| | CAUGACAUUGCAAGAGUUUCGCCCAGUUUCUUUUAAAGACCCAAGCUGAUUUAAGCGGCUG |
| | AAAUGAGAU (SEQ ID NO: 282) |
| | UACCGCAGUUUGGGCGGGUUGCUAAUAAUAUUAGCUUAUGACUUCUAAGGCUUUUGCCUAA |
| | AAAGUAAGGGGGAAGGCGACAACCCCCAAAGACCAGCCGGAACUAUGGCUGGCAACUAUCU |
| | CAUCUUUUUGGCAUAUCAAAGCUAGGGCAAAAACCCCAGACAUUCGCCAAAAGCCCAGAAC |
| | CAUGACAUUGCAAGAGUUUCGCCCAGUUUCUUUUAAAGACCCAAGCUGAUUUAAGCGGCUG |
| | AAAUGAGAU (SEQ ID NO: 283) |
| | UACCGCAGUUUGGGCGGGUUGCUAAUAAUAUUAGCUUAUGACUUCUAAGGCUUUUGCCUAA |
| | AAAGUAAGGGGGAAGGCGACAACCCCCAAAGACCAGCCGGAACUAUGGCUGGCAACUAUCU |
| | CAUCUUUUUGGCAUAUCAAAGCUAGGGCAAAAACCCCAGACAUUCGCCAAAAGCCCAGAAC |
| | CAUGACAUUGCAAGAGUUUCGCCCAGUUUCUUUUAAAGACCCAAGCUGAUUUAAGCGGCUG |
| | AAAUGAGAUUUUUUAACAGGUCCGCCAAAAUCGCUUCUG (SEQ ID NO: 284) |
| | UACCGCAGUUUGGGCGGGUUGCUAAUAAUAUUAGCUUAUGACUUCUAAGGCUUUUGCCUAA |
| | AAAGUAAGGGGGAAGGCGACAACCCCCAAAGACCAGCCGGAACUAUGGCUGGCAACUAUCU |
| | CAUCUUUUUGGCAUAUCAAAGCUAGGGCAAAAACCCCAGACAUUCGCCAAAAGCCCAGAAC |
| | CAUGACAUUGCAAGAGUUUCGCCCAGUUUCUUUUAAAGACCCAAGCUGAUUUAAGCGGCUG |
| | AAAUGAGAUUUUUUAACAGGUCCGCCAAAAUCGCUUCUG (SEQ ID NO: 285) |
| | AUAAGAUGGACUAUAUUUAUAAACCAGGAAACAUAGUUAUUACAAUUGCAUUGUAGGUUUC |
| | AACCUGAUACCCACUAAAGCGUGUUAGUGGGUAUUUUUU (SEQ ID NO: 286) |
| | AUAAGAUGGACUAUAUUUAUAAACCAGGAAACAUAGUUAUUACAAUUGCAUUGUAGGUUUC |
| | AACCUGAUACCCACUAAAGCGUGUUAGUGGGUAUUUUUU (SEQ ID NO: 287) |
| | CUUGACAGUAAACAAAAAAUAAGAUGGACUAUAUUUAUAAACCAGGAAACAUAGUUAUUAC |
| | AAUUGCAUUGUAGGUUUCAACCUGAUACCCACUAAAGCGUGUUAGUGGGUAUUUUUUAU |
| | (SEQ ID NO: 288) |
| | CUUGACAGUAAACAAAAAAUAAGAUGGACUAUAUUUAUAAACCAGGAAACAUAGUUAUUAC |
| | AAUUGCAUUGUAGGUUUCAACCUGAUACCCACUAAAGCGUGUUAGUGGGUAUUUUUUAU |
| | (SEQ ID NO: 289) |
| SEQ ID NO: 31 | AGCCGCACGGAACCUGAGCCGAUGGCGUAGCCCUUGGACCUAUAUGGAACGCGGCAUAAGC |
| | CCUGCGAGUUCGCAAGAGCCCAAGGCGGCAUGACAAGCCUCUUUCAGGCGACAGAGUCUUU |
| | UGGAGUGUCGAGGCUCCCUGCAUUCCUUGGGAGCCUCCC (SEQ ID NO: 290) |
| | AGCCGCACGGAACCUGAGCCGAUGGCGUAGCCCUUGGACCUAUAUGGAACGCGGCAUAAGC |
| | CCUGCGAGUUCGCAAGAGCCCAAGGCGGCAUGACAAGCCUCUUUCAGGCGACAGAGUCUUU |
| | UGGAGUGUCGAGGCUCCCUGCAUUCCUUGGGAGCCUCCC (SEQ ID NO: 291) |
| | AGCCGCACGGAACCUGAGCCGAUGGCGUAGCCCUUGGACCUAUAUGGAACGCGGCAUAAGC |
| | CCUGCGAGUUCGCAAGAGCCCAAGGCGGCAUG (SEQ ID NO: 292) |
| | AGCCGCACGGAACCUGAGCCGAUGGCGUAGCCCUUGGACCUAUAUGGAACGCGGCAUAAGC |
| | CCUGCGAGUUCGCAAGAGCCCAAGGCGGCAUG (SEQ ID NO: 293) |
| SEQ ID NO: 32 | GUUCCGUCUCGACUAUGCCGUACCACUAGACCGAGCCUACACGGCACGCGGUCAUAGCGUU |
| | AACCAAGGCGUGGUGACAAGCCUCUUUCAGGCGUCGGACACUUAAGAGCGUUAGGCGGGCG |
| | GUCCCUAAGCCGCCCGCCCCC (SEQ ID NO: 294) |
| | GUUCCGUCUCGACUAUGCCGUACCACUAGACCGAGCCUACACGGCACGCGGUCAUAGCGUU |
| | AACCAAGGCGUGGUGACAAGCCUCUUUCAGGCGUCGGACACUUAAGAGCGUUAGGCGGGCG |
| | GUCCCUAAGCCGCCCGCCCCC (SEQ ID NO: 295) |
| | GUUCCGUCUCGACUAUGCCGUACCACUAGACCGAGCCUACACGGCACGCGGUCAUAGCGUU |
| | AACCAAGGCGUGGUGACAAGCCUCUUUCAGGCGUCGGACACUUAAGAGCGUUAGGCGGGCG |
| | GUCCCUAAGCCGCCCGCCCCCUUAUUUGCACGUUUUCCCCGAACCCCGUAA (SEQ ID |
| | NO: 296) |
| | GUUCCGUCUCGACUAUGCCGUACCACUAGACCGAGCCUACACGGCACGCGGUCAUAGCGUU |
| | AACCAAGGCGUGGUGACAAGCCUCUUUCAGGCGUCGGACACUUAAGAGCGUUAGGCGGGCG |
| | GUCCCUAAGCCGCCCGCCCCCUUAUUUGCACGUUUUCCCCGAACCCCGUAA (SEQ ID |
| | NO: 297) |
| | GUUCCGUCUCGACUAUGCCGUACCACUAGACCGAGCCUACACGGCACGCGGUCAUAGCGUU |
| | AACCAAGGCGUGGUGACAAGCCUCUUUCAGGCGUCGGACACUUAAGAGCGUUA (SEQ ID |
| | NO: 298) |
| | GUUCCGUCUCGACUAUGCCGUACCACUAGACCGAGCCUACACGGCACGCGGUCAUAGCGUU |
| | AACCAAGGCGUGGUGACAAGCCUCUUUCAGGCGUCGGACACUUAAGAGCGUUA (SEQ ID |
| | NO: 299) |

TABLE 4
sgRNA sequences.

| Nuclease | |
|---|---|
| polypeptide | sgRNA Sequences |
| SEQ ID NO: 1 | UUUCGUUCGAGGCCGGGAGCAACGGACCGCUAGCCCAAGGGCAACCGCGGCCGCCCGGUCG |
| | AAGGCCGACCUGUACGGCCUGAAGGUUGAGAAGGCACGAAUAAGCGGAAAAACUCGUUUCC |
| | CUUCGUGUUCGCUCACCGAGCAGACGUCGCAAAGGCGACCUGCUUACAGG[spacer] |
| | [spacer] (SEQ ID NO: 300) |
| SEQ ID NO: 2 | GAGAGGGGUAUUCUGUUGAUGGGCGAAUCGGUAAAAGCAAUAAAAUUAAAGAUACUGGAUA |
| | UGUUUUUAGACCCCGAAUGCACAAAGCAGGAUGAUAACUGGCGCAAAGAUUUGUCUACUAU |
| | GUCCAGAUUCUGCGCUGAAGCCUUGCGUAAGCGCGUGGAUUGAAAC[spacer] (SEQ |
| | ID NO: 301) |
| | GAGAGGGGUAUUCUGUUGAUGGGCGAAUCGGUAAAAGCAAUAAAAUUAAAGAUACUGGAUA |
| | UGUUUUUAGACCCCGAAUGCACAAAGCAGGAUGAUAACUGGCGCAAAGAUUUGUCUACUAU |
| | GUCCAGAUUCUGCGCUGAAGCCUUGCGAAAGCGCGUGGAUUGAAAC[spacer] (SEQ |
| | ID NO: 302) |
| | GAGAGGGGUAUUCUGUUGAUGGGCGAAUCGGUAAAAGCAAUAAAAUUAAAGAUACUGGAUA |
| | UGUUUUUAGACCCCGAAUGCACAAAGCAGGAUGAUAACUGGCGCAAAGAUUUGUCUACUAU |
| | GUCCAGAUUCUGCGCUGAAGCGGGGAAUAUGUGUCUGCGCGACCUGCUUGCGUAAGCGCGU |
| | GGAUUGAAAC[spacer] (SEQ ID NO: 303) |
| | GAGAGGGGUAUUCUGUUGAUGGGCGAAUCGGUAAAAGCAAUAAAAUUAAAGAUACUGGAUA |
| | UGUUUUUAGACCCCGAAUGCACAAAGCAGGAUGAUAACUGGCGCAAAGAUUUGUCUACUAU |
| | GUCCAGAUUCUGCGCUGAAGCGGGGAAUAUGUGUCUGCGCGAAAAGUUGCGUAAGCGCGUG |
| | GAUUGAAAC[spacer] (SEQ ID NO: 304) |
| | UUUACUCUGUUUCGCGCGCCAGGGCAGUUAGGUGCCCUAAAAGAGCGAAGUGGCCGAAAGG |
| | AAAGGCUAACGCUUCUCUAACGCUACGGCGACCUUGGCGAAAUGCCAUCAAUACCACGCGG |
| | CCCGAAAGGGUUCGCGCGAAACUGAGUCUUGCGUAAGCGCGUGGAUUGAAAC [spacer] |
| | (SEQ ID NO: 305) |
| | UUUACUCUGUUUCGCGCGCCAGGGCAGUUAGGUGCCCUAAAAGAGCGAAGUGGCCGAAAGG |
| | AAAGGCUAACGCUUCUCUAACGCUACGGCGACCUUGGCGAAAUGCCAUCAAUACCACGCGG |
| | CCCGAAAGGGUUCGCGCGAAACUGAGUCUUGCGAAAGCGCGUGGAUUGAAAC[spacer] |
| | (SEQ ID NO: 306) |
| | UUUACUCUGUUUCGCGCGCCAGGGCAGUUAGGUGCCCUAAAAGAGCGAAGUGGCCGAAAGG |
| | AAAGGCUAACGCUUCUCUAACGCUACGGCGACCUUGGCGAAAUGCCAUCAAUACCACGCGC |
| | UUGCGUAAGCGCGUGGAUUGAAAC[spacer] (SEQ ID NO: 307) |
| | UUUACUCUGUUUCGCGCGCCAGGGCAGUUAGGUGCCCUAAAAGAGCGAAGUGGCCGAAAGG |
| | AAAGGCUAACGCUUCUCUAACGCUACGGCGACCUUGGCGAAAUGCCAUCAAUACCACGCGC |
| | UUAAAGAAGCGCGUGGAUUGAAAC[spacer] (SEQ ID NO: 308) |
| SEQ ID NO: 3 | GUCGCUAUCAGCGGCAAUGCAAUUUCCUCCGCCUUUGGAAAUUUACGUUCUAUGACUUUGA |
| | GGUAUGGCGG[spacer] (SEQ ID NO: 309) |
| | GUCGCUAUCAGCGGCAAUGCAAUUUCCUCCGCCUUUGGAAAAAGUUCUAUGACUUUGAGGU |
| | AUGGCGG[spacer] (SEQ ID NO: 310) |
| | GUCGCUAUCAGCGGCAAUGCAAUUUCCUCCGCCUUUGGAAAUUUACGUUCUAUGACUUUGC |
| | GAGAGCUGCGGUGCCCCGUUGCGGCCGGGAGGUAUGGCGG[spacer] (SEQ ID NO: |
| | 311) |
| | GUCGCUAUCAGCGGCAAUGCAAUUUCCUCCGCCUUUGGAAAUUUACGUUCUAUGACUUUGC |
| | GAGAGCUGCGGAAAGCCGUUGCGGCCGGGAGGUAUGGCGG[spacer] (SEQ ID NO: |
| | 312) |
| | AUACGUCGCGACAUUCCAGCAUGUAACCCGUCGGGAGGAUUGCCCCAUGAACCGUAUUUAC |
| | CAAGGUCGGAUUACCGGCAUUCUUGAUUCCAAGGAGGACGAGCGGGGCCACCCUCCCCCAC |
| | CAGGUAUGGCGG[spacer] (SEQ ID NO: 313) |
| | AUACGUCGCGACAUUCCAGCAUGUAACCCGUCGGGAGGAUUGCCCCAUGAACCGUAUUUAC |
| | CAAGGUCGGAUUACCGGCAUUCUUGAUUCCAAGGAGGACGAGCGGGGCCACCCUCCCCCAA |
| | AGGGUAUGGCGG[spacer] (SEQ ID NO: 314) |
| | AUACGUCGCGACAUUCCAGCAUGUAACCCGUCGGGAGGAUUGCCCCAUGAACCGUAUUUAC |
| | CAAGGUCGGAUUACCGGCAUUCUUGAUUCCAAGGAGGACGAGCGGGGCCACCCUCCCCCAC |
| | CCGAUCCGAAACACAAUCCAUUCUGGCGGCAAGGUAUGGCGG[spacer] (SEQ ID |
| | NO: 315) |
| | AUACGUCGCGACAUUCCAGCAUGUAACCCGUCGGGAGGAUUGCCCCAUGAACCGUAUUUAC |
| | CAAGGUCGGAUUACCGGCAUUCUUGAUUCCAAGGAGGACGAGCGGGGAAAGCCCCACCCGA |
| | UCCGAAACACAAUCCAUUCUGGCGGCAAGGUAUGGCGG[spacer] (SEQ ID NO: |
| | 316) |
| | CUUGACAUGGCUUAAGAAUGCGCCAUGUAUGAAGGAGAAGUAACCUGCUUCCUUCCUCGCU |
| | GUCACCGCCUUCGUCAGCGAUUUCAUCGGCGGAACAUAAUAAUUUUACUUUUUUCUAGGUA |
| | UGGCGG[spacer] (SEQ ID NO: 317) |
| | CUUGACAUGGCUUAAGAAUGCGCCAUGUAUGAAGGAGAAGUAACCUGCUUCCUUCCUCGCU |
| | GUCACCGCCUUCGUCAGCGAUUUCAUCGGCGGAACAUAAUAAUUUUACUUAAAGAGGUAUG |
| | GCGG[spacer] (SEQ ID NO: 318) |
| SEQ ID NO: 4 | GGUAUAUCCGUUUUGUUCUGCUCUCUAUCGCAUUCGGGGGGCAAUGCGUCAAAGAUUAGUG |
| | ACGUGUUUGCGAAAUAGACGUCUAUUGAUAUUACUGAAUGGCGG[spacer] (SEQ ID |
| | NO: 319) |
| | GGUAUAUCCGUUUUGUUCUGCUCUCUAUCGCAUUCGGGGGGCAAUGCGUCAAAGAUUAGUG |
| | ACGUGUUUGCGAAAUAGAAAGCUAUUGAUAUUACUGAAUGGCGG spacer] (SEQ ID |
| | NO: 320) |
| SEQ ID NO: 5 | AAAGUGUACGUCUUUUUGUACAACCUCUUCGGCCUUCUGGCUACGGGUGCCCUGGAUCACG |
| | CCGCUGUAGAAGGGCGUCCAUUCACGGCUGACGGAAAC[spacer] (SEQ ID NO: |
| | 321) |
| | AAAGUGUACGUCUUUUUGUACAACCUCUUCGGCCUUCUGGCUACGGGUGCCCUGGAUCACG |
| | CCGCUAAAGAGGGCGUCCAUUCACGGCUGACGGAAAC[spacer] (SEQ ID NO: |
| | 322) |
| | AAAGUGUACGUCUUUUUGUACAACCUCUUCGGCCUUCUGGCUACGGGUGCCCUGGAUCACG |
| | CCUUUGGGACCUGGGAAACCACAACCUUUCCCGCUGUAGAAGGGCGUCCAUUCACGGCUGA |
| | CGGAAAC[spacer] (SEQ ID NO: 323) |
| | AAAGUGUACGUCUUUUUGUACAACCUCUUCGGCCUUCUGGCUACGGGUGCCCUGGAUCACG |
| | CCUUUGGGACCUGGGAAAAAAGUUUCCCGCUGUAGAAGGGCGUCCAUUCACGGCUGACGGA |
| | AAC[spacer] (SEQ ID NO: 324) |
| | AAAUCUCGGUCGAUGGAAAUGUUGCCCUCUUUGGUGAUGUGAUAGCUGUAGAAGGGCGUCC |
| | AUUCACGGCUGACGGAAAC[spacer] (SEQ ID NO: 325) |
| | AAAUCUCGGUCGAUGGAAAUGUUGCCCUCUUUGGUAAAGGCUGUAGAAGGGCGUCCAUUCA |
| | CGGCUGACGGAAAC[spacer] (SEQ ID NO: 326) |
| | CGAUUCCACCUAACUGUCUGACAGGAAACUGCUUCGCCGCAUUGAUGCUUCGAGCUGUAGA |
| | AGGGCGUCCAUUCACGGCUGACGGAAAC[spacer] (SEQ ID NO: 327) |
| | CGAUUCCACCUAACUGUCUGACAGGAAACUGCUUCGCCGCAUUGAUGCUUCGAGCUGUAGA |
| | AGGAAAGCCAUUCACGGCUGACGGAAAC[spacer] (SEQ ID NO: 328) |
| | AAAUUAUCGGCCGGGACGGCUCCUUUGCCCCAGGGCAGGUGAGCCCGUAGUUUCGCGGCUG |
| | UAGAAGGGCGUCCAUUCACGGCUGACGGAAAC[spacer] (SEQ ID NO: 329) |
| | AAAUUAUCGGCCGGGACGGCUCCUUUGCCCCAGGGCAGGUGAGCCCGUAGUUUCGCGGCUA |
| | AAGGGCGUCCAUUCACGGCUGACGGAAAC[spacer] (SEQ ID NO: 330) |
| | AAAUUAUCGGCCGGGACGGCUCCUUUGCCCCAGGGCAGGUGAGCCCGUAGUUUCGCGGCCG |
| | GGUACUGGACGGAUAGGCGAAUGACGGCUGUAGAAGGGCGUCCAUUCACGGCUGACGGAAA |
| | C[spacer] (SEQ ID NO: 331) |
| | AAAUUAUCGGCCGGGACGGCUCCUUUGCCCCAGGGCAGGUGAGCCCGUAGUUUCGCGGCCG |
| | GGUACUGGACGGAUAGGCGAAUGACGGCUAAAGGGGCGUCCAUUCACGGCUGACGGAAAC |
| | [spacer] (SEQ ID NO: 332) |
| | GUUCGAUCUCUUUCAACAAUGGUUGAAGGAAAUUAUCGGCCGGGACGGCUCCUUUGCCCCA |
| | GGGCAGGUGAGCCCGUAGUUUCGCGGCCGGGUACUGGACGGGCUGUAGAAGGGCGUCCAUU |
| | CACGGCUGAOGGAAAC[spacer] (SEQ ID NO: 333) |
| | GUUCGAUCUCUUUCAACAAUGGUUGAAGGAAAUUAUCGGCCGGGACGGCUCCUUUGCCCCA |
| | GGGCAGGUGAGCCCGUAGUUUCGCGGCCGGAAAGCGGGCUGUAGAAGGGCGUCCAUUCACG |
| | GCUGACGGAAAC[spacer] (SEQ ID NO: 334) |
| SEQ ID NO: 6 | GUUCCCAGCCUUCCAGGAAUGGUUGGUCGGGAUCAGACGCCGGGUUGGUGAGCCAGCUUGU |
| | UGCAACGGCUUCCGGAGUGCUGGUGGGAAUGAAAC[spacer] (SEQ ID NO: 335) |
| | GUUCCCAGCCUUCCAGGAAUGGUUGGUCGGGAUCAGACGCCGGGUUGGUGAGCCAGCAAAG |
| | GCAACGGCUUCCGGAGUGCUGGUGGGAAUGAAAC [spacer] (SEQ ID NO: 336) |
| | GUUCCCAGCCUUCCAGGAAUGGUUGGUCGGGAUCAGACGCCGGGUUGGUGAGCCAGCUUUA |
| | AUCGGCCGGUGAGCCAGGUAGUUUCAUCGUUGCAACGGCUUCCGGAGUGCUGGUGGGAAUG |
| | AAAC[spacer] (SEQ ID NO: 337) |
| | GUUCCCAGCCUUCCAGGAAUGGUUGGUCGGGAUCAGACGCCGGGUUGGUGAGCCAGCUAAA |
| | GGGCCGGUGAGCCAGGUAGUUUCAUCGUUGCAACGGCUUCCGGAGUGCUGGUGGGAAUGAA |
| | AC[spacer] (SEQ ID NO: 338) |
| | AGCUUAGGCUGAAGGGAGGCGCUGCAUUCUGAGUCGGGUCCGGAGCUGUUGCAACGGCUUC |
| | CGGAGUGCUGGUGGGAAUGAAACAUGAUCUCUUUUCUCCGUUGCAACGGCUUCCGGAGUGC |
| | UGGUGGGAAUGAAAC[spacer] (SEQ ID NO: 339) |
| | AGCUUAGGCUGAAGGGAGGCGCUGCAUUCUGAGUCGGGUCCGGAGCUGUUGCAACGGCUUC |
| | CGGAGUGCUGGUAAAGACAUGAUCUCUUUUCUCCGUUGCAACGGCUUCCGGAGUGCUGGUG |
| | GGAAUGAAAC[spacer] (SEQ ID NO: 340) |
| SEQ ID NO: 7 | GUUCGAUCUCUUUCAACAGUGGUUGAAGGAGCUUAUCGGCCGGGACGGUUCCUCUGCCUUC |
| | GGGCAAGGGAACCAGUAGUUUCGCGGCCGGGUACUGGGCGGAUAGGCGAAUGACGGCAGCC |
| | UCACGGCUGGCGCGCUGUAGAGGGGCGUCCAUUCACGGCUGACGGAAAC[spacer] |
| | (SEQ ID NO: 341) |
| | GUUCGAUCUCUUUCAACAGUGGUUGAAGGAGCUUAUCGGCCGGGACGGUUCCUCUGCCUUC |
| | GGGCAAGGGAACCAGUAGUUUCGCGGCCGGGUACUGGGCGGAUAGGCGAAUGACGGCAGCC |
| | AAAGGGCUGGCGCGCUGUAGAGGGGCGUCCAUUCACGGCUGACGGAAAC[spacer] |
| | (SEQ ID NO: 342) |
| | GUUCGAUCUCUUUCAACAGUGGUUGAAGGAGCUUAUCGGCCGGGACGGUUCCUCUGCCUUC |
| | GGGCAAGGGAACCAGUAGUUUCGCGGCCGGGUACUGGGCGGAUAGGCGAAUGACGGCAGCC |
| | UCACGGCUGGCGCGUAACCGACCAUCCCCUGGUCCGAACAGCCGCUGUAGAGGGGCGUCCA |
| | UUCACGGCUGACGGAAAC[spacer] (SEQ ID NO: 343) |
| | GUUCGAUCUCUUUCAACAGUGGUUGAAGGAGCUUAUCGGCCGGGACGGUUCCUCUGCCUUC |
| | GGGCAAGGGAACCAGUAGUUUCGCGGCCGGGUACUGGGCGGAUAGGCGAAUGACGGCAGCC |
| | UCACGGCUGGCGCGUAACCGACCAUCCCCUGGUCCGAACAGCCGCUGUAGAGGAAAGCCAU |
| | UCACGGCUGACGGAAAC[spacer] (SEQ ID NO: 344) |
| | GUUCGAUCUCUUUCAACAGUGGUUGAAGGAGCUUAUCGGCCGGGACGGUUCCUCUGCCUUC |
| | GGGCAAGGGAACCAGUAGUUUCGCGGCCGGGUACUGGGCGCUGUAGAGGGGCGUCCAUUCA |
| | CGGCUGACGGAAAC[spacer] (SEQ ID NO: 345) |
| | GUUCGAUCUCUUUCAACAGUGGUUGAAGGAGCUUAUCGGCCGGGACGGUUCCUCUGCCUUC |
| | GGGCAAGGGAACCAGUAGUUUCGCGGCCGGGUACUGGGCGCUAAAGGGCGUCCAUUCACGG |
| | CUGAOGGAAAC[spacer] (SEQ ID NO: 346) |
| SEQ ID NO: 8 | GUUCGAUCUCUUUCAGCAAUGGUUGAAGGAGCUUAUCGGCCGGGACGGUUCUGUUGCCCUC |
| | GGGCAGGGGAACCAGUAGUUUCGCGGCCGGGUACUGGGCUGUAUAAGGGCGUCCAUUCACG |
| | GCUGACGGAAAC[spacer] (SEQ ID NO: 347) |
| | GUUCGAUCUCUUUCAGCAAUGGUUGAAGGAGCUUAUCGGCCGGGACGGUUCUGUUGCCCUC |
| | GGGCAGGGGAACCAGUAGUUUCGCGGCCGGGUACUGGGCUGUAAAGGCGUCCAUUCACGGC |
| | UGACGGAAAC[spacer] (SEQ ID NO: 348) |
| SEQ ID NO: 9 | GAAGUUCGAUCUCUUUCAACAGUGGUUGAAGGAGCUUAUCGGCCGGGACGGUUCCGCUGCC |
| | CCAGGGUAGUGGAACCAGUAGUUUCGCGGCCGGGUACUGGGCGGAUAGGCGAAUGACGGCA |
| | UCCUCACGGUUGCCGCGCUGUAGAGGGGCGUCCAUUCACGGCUGACGGAAAC[spacer] |
| | (SEQ DI NO: 349) |
| | GAAGUUCGAUCUCUUUCAACAGUGGUUGAAGGAGCUUAUCGGCCGGGACGGUUCCGCUGCC |
| | CCAGGGUAGUGGAACCAGUAGUUUCGCGGCCGGGUACUGGGCGGAUAGGCGAAAGCGGCAU |
| | CCUCACGGUUGCCGCGCUGUAGAGGGGCGUCCAUUCACGGCUGACGGAAAC[spacer] |
| | (SEQ ID NO: 350) |
| | GAAGUUCGAUCUCUUUCAACAGUGGUUGAAGGAGCUUAUCGGCCGGGACGGUUCCGCUGCC |
| | CCAGGGUAGUGGAACCAGUAGUUUCGCGGCCGGGUACUGGGCGGAUAGGCGAAUGACGGCA |
| | UCCUCACGGUUGCCGCGUAACCGACCAUCCCCUGGUCCGAACAGCCGCUGUAGAGGGGCGU |
| | CCAUUCACGGCUGACGGAAAC[spacer] (SEQ ID NO: 351) |
| | GAAGUUCGAUCUCUUUCAACAGUGGUUGAAGGAGCUUAUCGGCCGGGACGGUUCCGCUGCC |
| | CCAGGGUAGUGGAACCAGUAGUUUCGCGGCCGGGUACUGGGCGGAUAGGCGAAUGACGGCA |
| | UCCUCACGGUUGCCGCGUAACCGACCAUCCCCUGGUCCGAACAGCCGCUGUAGAGGAAAGC |
| | CAUUCACGGCUGACGGAAAC[spacer] (SEQ ID NO: 352) |
| | GAAGUUCGAUCUCUUUCAACAGUGGUUGAAGGAGCUUAUCGGCCGGGACGGUUCCGCUGCC |
| | CCAGGGUAGUGGAACCAGUAGUUUCGCGGCCGGGUACUGGGCGCUGUAGAGGGGCGUCCAU |
| | UCACGGCUGACGGAAAC[spacer] (SEQ ID NO: 353) |
| | GAAGUUCGAUCUCUUUCAACAGUGGUUGAAGGAGCUUAUCGGCCGGGACGGUUCCGCUGCC |
| | CCAGGGUAGUGGAACCAGUAGUUUCGCGGCCGGGUACUGGGCGCUAAAGGGCGUCCAUUCA |
| | CGGCUGAOGGAAAC[spacer] (SEQ ID NO: 354) |
| SEQ ID NO: 10 | GUUCGAUCUCUUUCAACUGUGGUUGAAGGAGCUUAUCGGCCGGGACGGUUCCAUUGCCUCA |
| | GGGCAGGGGAACCAGUAGUUUCGCGGCCGGGUACUGGGCGGAUAGGCGAAUGAUGGCAUCC |
| | UCACGGUUGCCGCCUGACGGAAAC[spacer] (SEQ ID NO: 355) |
| | GUUCGAUCUCUUUCAACUGUGGUUGAAGGAGCUUAUCGGCCGGGACGGUUCCAUUGCCUCA |
| | GGGCAGGGGAACCAGUAGUUUCGCGGCCGGGUACUGGGCGGAUAGGCGAAUGAUGGAAAGC |
| | CUCACGGUUGCCGCCUGACGGAAAC[spacer] (SEQ ID NO: 356) |
| | GUUCGAUCUCUUUCAACUGUGGUUGAAGGAGCUUAUCGGCCGGGACGGUUCCAUUGCCUCA |
| | GGGCAGGGGAACCAGUAGUUUCGCGGCCGGGUACUGGGCGGAUAGGCGAAUGAUGGCAUCC |
| | UCACGGUUGCCGCGUAACCGACCAUCCCCUGGUCCGAACAGCCCUGACGGAAAC[spacer] |
| | (SEQ ID NO: 357) |
| | GUUCGAUCUCUUUCAACUGUGGUUGAAGGAGCUUAUCGGCCGGGACGGUUCCAUUGCCUCA |
| | GGGCAGGGGAACCAGUAGUUUCGCGGCCGGGUACUGGGCGGAUAGGCGAAUGAUGGCAUCC |
| | UCACGGUUGCCGCGUAACCGACCAUCCCCUGGUCCGAACAGCCCAAAGGGAAAC[spacer] |
| | (SEQ ID NO: 358) |
| | AUAUCUCAAGCCAUGUUCAAAACGGCGCGUUUCAAGGUUCACAAUCCGUCACGGCACAAGA |
| | GCACUAUGCUCUGGUAUGCCAUGACCCGCUAUCACGAGACUUUGAAGGACGUACUCGAAAA |
| | GACACUGGCGAUUCCAGAUCUGCUAGAACACUGACGGAAAC[spacer] (SEQ ID NO: |
| | 359) |
| | AUAUCUCAAGCCAUGUUCAAAACGGCGCGUUUCAAGGUUCACAAUCCGUCACGGCACAAGA |
| | GCACUAUGCUCUGGUAUGCCAUGACCCGCUAUCACGAGACUUUGAAGGACGUACUCGAAAA |
| | GACACUGGCGAUUCCAGAUCUGCUAGAAAGCUGACGGAAAC[spacer] (SEQ ID NO: |
| | 360) |
| | AUAUCUCAAGCCAUGUUCAAAACGGCGCGUUUCAAGGUUCACAAUCCGUCACGGCACAAGA |
| | GCACUAUGCUCUGGUAUGCCAUGACCCGCUAUCACGAGACUUUGAAGGACGUACUCGAAAA |
| | GACACUGGCGAUUCCAGAUCUGCUAGAACAAAUCUCAGAACUGACGGAAAC[spacer] |
| | (SEQ ID NO: 361) |
| | AUAUCUCAAGCCAUGUUCAAAACGGCGCGUUUCAAGGUUCACAAUCCGUCACGGCACAAGA |
| | GCACUAUGCUCUGGUAUGCCAUGACCCGCUAUCACGAGACUUUGAAGGACGUACUCGAAAA |
| | GACACUGGCGAUUCCAGAUCUGCUAGAAAAGUCUCAGAACUGACGGAAAC[spacer] |
| | (SEQ ID NO: 362) |
| | GUUCGAUCUCUUUCAACUGUGGUUGAAGGAGCUUAUCGGCCGGGACGGUUCCAUUGCCUCA |
| | GGGCAGGGGAACCAGUAGUUUCGCGGCCGGGUACUGGGCCUGACGGAAAC[spacer] |
| | (SEQ ID NO: 363) |
| | GUUCGAUCUCUUUCAACUGUGGUUGAAGGAGCUUAUCGGCCGGGACGGUUCCAUUGCCUCA |
| | GGGCAGGGGAACCAGUAGUUUCGCGGCCGGGUAAAGGCCUGACGGAAAC[spacer] |
| | (SEQ ID NO: 364) |
| SEQ ID NO: 11 | CAAAUUCGGUGCAAGGGCGAAGCCCAACCGGUAGACUCAUAUCUUCCGGUAACGCACCUCG |
| | GCUGUUUAGACCUGUAGAAGGGCGUCCAUUCACGGCUGACGGAAAC[spacer] (SEQ |
| | ID NO: 365) |
| | CAAAUUCGGUGCAAGGGCGAAGCCCAACCGGUAGACUCAUAUCUUCCGGUAACGCACCUCG |
| | GCUGUUUAGACCUAAAGGGCGUCCAUUCACGGCUGACGGAAAC[spacer] (SEQ ID |
| | NO: 366) |
| | CAAAUUCGGUGCAAGGGCGAAGCCCAACCGGUAGACUCAUAUCUUCCGGUAACGCACCUCG |
| | GCUGUUUAGACCAGAGGAUGGUCGGUUACGCGGCAACCGUGAUGUAGAAGGGCGUCCAUUC |
| | ACGGCUGACGGAAAC[spacer] (SEQ ID NO: 367) |
| | CAAAUUCGGUGCAAGGGCGAAGCCCAACCGGUAGACUCAUAUCUUCCGGUAACGCACCUCG |
| | GCUGUUUAGACCAGAGGAUGGUCGGUUACGCGGCAACCGUGAUGUAGAAGGAAAGCCAUUC |
| | ACGGCUGACGGAAAC[spacer] (SEQ ID NO: 368) |
| | GUUCGAUAUCUUUCAACAGUGGUUGAAGGAGCUUAUCGGCCGGGACGGUUCCUCUGCCCCA |
| | GGGCAGUUGAACCCGUAGUUUCGCGGCCGGGUACUGGGCUGUAGAAGGGCGUCCAUUCACG |
| | GCUGACGGAAAC[spacer] (SEQ ID NO: 369) |
| | GUUCGAUAUCUUUCAACAGUGGUUGAAGGAGCUUAUCGGCCGGGACGGUUCCUCUGCCCCA |
| | GGGCAGUUGAACCCGUAGUUUCGCGGCCGGGUACUGGGCUGUAAAGGCGUCCAUUCACGGC |
| | UGACGGAAAC[spacer] (SEQ ID NO: 370) |
| SEQ ID NO: 12 | AUUGAUGAGGCUGUCUGACUUGUGCAGGCUCAUUAUUGAUAGGAUUGAUGAGGAUGACUUA |
| | AAACGCGAAACCUCAUCAAUCCUAUCAAUAAUGAG[spacer] (SEQ ID NO: 371) |
| | AUUGAUGAGGCUGUCUGACUUGUGCAGGCUCAUUAUUGAUAGGAUUGAUGAGGAAAGCCUC |
| | AUCAAUCCUAUCAAUAAUGAG[spacer] (SEQ ID NO: 372) |
| | AUUGAUGAGGCUGUCUGACUUGUGCAGGCUCAUUAUUGAUAGGAUUGAUGAGGAUGACUUA |
| | AAACGCGAAACUCAUUAUUGAUAGGAUUGAUGAGGAUUUUCCUCAUCAAUCCUAUCAAUAA |
| | UGAG[spacer] (SEQ ID NO: 373) |
| | AUUGAUGAGGCUGUCUGACUUGUGCAGGCUCAUUAUUGAUAGGAUUGAUGAGGAUGACUUA |
| | AAACGCGAAACUCAUUAUUGAUAGGAUUGAUGAGGAAAAGUCCUCAUCAAUCCUAUCAAUA |
| | AUGAG[spacer] (SEQ ID NO: 374) |
| | ACAAUCCCUCUUGAGCAUUGGAUGCAGAUUCUUCUGUCUCCGUACCUCGCUCAUAAGGGCG |
| | CUGUCGGGAUAGCAGUGCCUAUCAAAACCCUCAUCAAUCCUAUCAAUAAUGAG[spacer] |
| | (SEQ ID NO: 375) |
| | ACAAUCCCUCUUGAGCAUUGGAUGCAGAUUCUUCUGUCUCCGUACCUCGCUCAUAAGGGCG |
| | CUGUCGGGAUAGCAGUGCCUAUCAAAACCCUCAUAAAGAUGAG[spacer] (SEQ ID |
| | NO: 376) |
| | ACAAUCCCUCUUGAGCAUUGGAUGCAGAUUCUUCUGUCUCCGUACCUCGCUCAUAAGGGCG |
| | CUGUCGGGAUAGCAGUGCCUAUCAAAACUAUCCCACUAUAAUGCUUGUUUCUUUACCUCCU |
| | CAUCAAUCCUAUCAAUAAUGAG[spacer] (SEQ ID NO: 377) |
| | ACAAUCCCUCUUGAGCAUUGGAUGCAGAUUCUUCUGUCUCCGUACCUCGCUCAUAAGGGCG |
| | CUGUCGGGAUAGCAGUGCCUAUCAAAACUAUCCCACUAUAAUGCUUGUUUCUUUACCUCCU |
| | CAUAAAGAUGAG[spacer] (SEQ ID NO: 378) |
| | UAUUGAUAGGAUUGAUGAGGUCGAGCAUCGAAAAGAUUCCUCAUCAAUCCUAUCAAUAAUG |
| | AG[spacer] (SEQ ID NO: 379) |
| | UAUUGAUAGGAUUGAUGAGGUCGAAAAGUCGAAAAGAUUCCUCAUCAAUCCUAUCAAUAAU |
| | GAG[spacer] (SEQ ID NO: 380) |
| | UAUUGAUAGGAUUGAUGAGGUCGAGCAUCGAAAAGAUUCUCAUUAUUGAUAGGAUUGAUGA |
| | GGCUUAACCUCAUCAAUCCUAUCAAUAAUGAG[spacer] (SEQ ID NO: 381) |
| | UAUUGAUAGGAUUGAUGAGGUCGAGCAUCGAAAAGAUUCUCAUUAUUGAUAGGAUUGAUGA |
| | GGAAAGCCUCAUCAAUCCUAUCAAUAAUGAG[spacer] (SEQ ID NO: 382) |
| SEQ ID NO: 13 | CUCAAACUCCUUCCGGCUUUCGGACCGUCUGCUUGCAGACCUCCUUACCUUGGCCGGAGGG |
| | ACAUCGCCGGGGGUCGAUGCCUGUCACUUACCCCCAUUCCUUCAAAACCCUGUCACAUCUG |
| | GA[spacer] (SEQ ID NO: 383) |
| | CUCAAACUCCUUCCGGCUUUCGGACCGUCUGCUUGCAGACCUCCUUACCUUGGCCGGAGGG |
| | ACAUCGCCGGGGGUCGAUGCCUGUCACUUACCCCCAUUCCAAAGGGA[spacer] (SEQ |
| | ID NO: 384) |
| | CUCAAACUCCUUCCGGCUUUCGGACCGUCUGCUUGCAGACCUCCUUACCUUGGCCGGAGGG |
| | ACAUCGCCGGGGGUCGAUGCCUGUCACUUACCCCCAUUUCACCCACUUCUUGGCUUGGUUU |
| | CUCCGGGCCUUCAAAACCCUGUCACAUCUGGA[spacer] (SEQ ID NO: 385) |
| | CUCAAACUCCUUCCGGCUUUCGGACCGUCUGCUUGCAGACCUCCUUACCUUGGCCGGAGGG |
| | ACAUCGCCGGGGGUCGAUGCCUGUCACUUACCCCCAUUUCACCCACUUCUUGGCUUGGUUU |
| | CUCCGGGCCUUCAAAACCCUGAAAGCUGGA[spacer] (SEQ ID NO: 386) |
| | GCACUGACACGCCCAGAAAUGACAGGGUUCUAAUCGGCUGAUCCCGCCUUGAAUUGACCAG |
| | AGGCCGUAAGGAUUCAAAAGGACUUCAAUGCGGCCUCAUGUCUCUCCUUCAAAACCCUGUC |
| | ACAUCUGGA[spacer] (SEQ ID NO: 387) |
| | GCACUGACACGCCCAGAAAUGACAGGGUUCUAAUCGGCUGAUCCCGCCUUGAAUUGACCAG |
| | AGGCCGUAAGGAUUCAAAGGACUUCAAUGCGGCCUCAUGUCUCUCCUUCAAAACCCUGUCA |
| | CAUCUGGA[spacer] (SEQ ID NO: 388) |
| | GCACUGACACGCCCAGAAAUGACAGGGUUCUAAUCGGCUGAUCCCGCCUUGAAUUGACCAG |
| | AGGCCGUAAGGAUUCAAAAGGACUUCAAUGCGGCCUCAUGUCUCUCUCCGGUUCGACCGGC |
| | UACGGGAAGGUUCUCCUUCAAAACCCUGUCACAUCUGGA[spacer] (SEQ ID NO: |
| | 389) |
| | GCACUGACACGCCCAGAAAUGACAGGGUUCUAAUCGGCUGAUCCCGCCUUGAAUUGACCAG |
| | AGGCCGUAAGGAUUCAAAAGGACUUCAAUGCGGCCUCAUGUCUCUCUCCGGUUCGACCGGC |
| | UACGGGAAGGAAAGCCUUCAAAACCCUGUCACAUCUGGA[spacer] (SEQ ID NO: |
| | 390) |
| SEQ ID NO: 14 | UCAGAAACUCCCGACUUCCUCAGGAUGAAGUUGAUUCUUUAUCCAUACCUUGGUGCCGGGA |
| | CGCCGAUUGAGGAAUGGGCGGCGCCUUCCAAAUUCUCAUUCCUCACUCCUUAUAAACCCUU |
| | CCAAUAAUGGG[spacer] (SEQ ID NO: 391) |
| | UCAGAAACUCCCGACUUCCUCAGGAUGAAGUUGAUUCUUUAUCCAUACCUUGGUGCCGGGA |
| | CGCCGAUUGAGGAAUGGGCGGCGCCUUCCAAAUUCUCAUUCCUCACUCCUUAUAAACCCAA |
| | AGGGG[spacer] (SEQ ID NO: 392) |
| | UCAGAAACUCCCGACUUCCUCAGGAUGAAGUUGAUUCUUUAUCCAUACCUUGGUGCCGGGA |
| | CGCCGAUUGAGGAAUGGGCGGCGCCUUCCAAAUUCUCAUUCCUCACUUUCCUCCUGCGAAA |
| | CUUCAUCGAAGCAAAUCCUUAUAAACCCUUCCAAUAAUGGG[spacer] (SEQ ID NO: |
| | 393) |
| | UCAGAAACUCCCGACUUCCUCAGGAUGAAGUUGAUUCUUUAUCCAUACCUUGGUGCCGGGA |
| | CGCCGAUUGAGGAAUGGGCGGCGCCUUCCAAAUUCUCAUUCCUCACUUUCCUCCUGCGAAA |
| | CUUCAUCGAAGCAAAUCCUUAUAAACCCAAAGGGG[spacer] (SEQ ID NO: 394) |
| | CAGAAACUCCCGACUUCCUCAGGAUGAAGUUGAUUCUUUAUCCAUACCUUGGUGCCGGGAC |
| | GCCGAUUGAGGAAUGGGCGGCGCCUUCCAAAUUCUCAUUCCUCACUUCCUUAUAAACCCUU |
| | CCAAUAAUGGG[spacer] (SEQ ID NO: 395) |
| | CAGAAACUCCCGACUUCCUCAGGAUGAAGUUGAUUCUUUAUCCAUACCUUGGUGCCGGGAC |
| | GCCGAUUGAGGAAUGGGCGGCGCCUUCCAAAUUCUCAUUCCUCACUUCCUUAUAAACCCAA |
| | AGGGG[spacer] (SEQ ID NO: 396) |
| | CAGAAACUCCCGACUUCCUCAGGAUGAAGUUGAUUCUUUAUCCAUACCUUGGUGCCGGGAC |
| | GCCGAUUGAGGAAUGGGCGGCGCCUUCCAAAUUCUCAUUCCUCACUUUCCUCCUGCGAAAC |
| | UUCAUCGAAGCAAAUUCCUUAUAAACCCUUCCAAUAAUGGG[spacer] (SEQ ID NO: |
| | 397) |
| | CAGAAACUCCCGACUUCCUCAGGAUGAAGUUGAUUCUUUAUCCAUACCUUGGUGCCGGGAC |
| | GCCGAUUGAGGAAUGGGCGGCGCCUUCCAAAUUCUCAUUCCUCACUUUCCUCCUGCGAAAC |
| | UUCAUCGAAGCAAAUUCCUUAUAAACCCAAAGGGG[spacer] (SEQ ID NO: 398) |
| SEQ ID NO: 15 | ACUAGUUAAAGAGGAGAAUAGAUUAUGGAACUAACAGAAGAACAGCACCAAGAUUUCAUUU |
| | GGACUUGGCCCUCGGUCCACUCCAGAGGUAAAGCCAGUCUCUGGUUACCUUAUUUUCAAGG |
| | UUUAGAGAAGUUAUCUUAUGACGAACUGAGG[spacer] (SEQ ID NO: 399) |
| | ACUAGUUAAAGAGGAGAAUAGAUUAUGGAACUAACAGAAGAACAGCACCAAGAUUUCAUUU |
| | GGACUUGGCCCUCGGUCCACUCCAGAGGUAAAGCCAGUCUCUGGUUACCUUAAAGAAGGUU |
| | UAGAGAAGUUAUCUUAUGACGAACUGAGG[spacer] (SEQ ID NO: 400) |
| | ACUAGUUAAAGAGGAGAAUAGAUUAUGGAACUAACAGAAGAACAGCACCAAGAUUUCAUUU |
| | GGACUUGGCCCUCGGUCCACUCCAGAGGUAAAGCCAGUCUCUGGUUACCUUAUUUUCAAGG |
| | UUUAGAGAAGUUGGGUCGCGGUAAGCCUUACCAGUAUAAGUUAUCUUAUGACGAACUGAGG |
| | [spacer] (SEQ ID NO: 401) |
| | ACUAGUUAAAGAGGAGAAUAGAUUAUGGAACUAACAGAAGAACAGCACCAAGAUUUCAUUU |
| | GGACUUGGCCCUCGGUCCACUCCAGAGGUAAAGCCAGUCUCUGGUUACCUUAUUUUCAAGG |
| | UUUAGAGAAGUUGGGUCGCGGUAAAAAGUUACCAGUAUAAGUUAUCUUAUGAGGAACUGAG |
| | G[spacer] (SEQ ID NO: 402) |
| | AGUUCGAAGGUAGUUGUUGAUUGAUCGGGUCAAACCAAUAAUCAGCCUAGGAACAAUUUUG |
| | AAUAUCUUAUGACGAACUGAGG[spacer] (SEQ ID NO: 403) |
| | AGUUCGAAGGUACUUGUUGAUUGAUCGGGUCAAACCAAUAAUCAGCCUAGGAACAAUUUUG |
| | AAUAUCUUAAAAGUGAGG[spacer] (SEQ ID NO: 404) |
| SEQ ID NO: 16 | AUGGGGUUUGGUAUAUCUUCCGCUCCAUUAUCUUGGUGUAAAGUGUAUGAGCCUGUUCACG |
| | GUUACGUAGGUGAUAUGGAAG[spacer] (SEQ ID NO: 405) |
| | AUGGGGUUUGGUAUAUCUUCCGCUCCAUUAUCUUGGUGUAAAGUGUAUGAGCCUGUUCACG |
| | GUUACAAAGGUGAUAUGGAAG[spacer] (SEQ ID NO: 406) |
| | AUGGGGUUUGGUAUAUCUUCCGCUCCAUUAUCUUGGUGUAAAGUGUAUGAGCCUAUCUUGG |
| | CCUUGCUCUACUCUAGGUUAGAAAGUGCUCUCAACCUGUUCACGGUUACGUAGGUGAUAUG |
| | GAAG[spacer] (SEQ ID NO: 407) |
| | AUGGGGUUUGGUAUAUCUUCCGCUCCAUUAUCUUGGUGUAAAGUGUAUGAGCCUAUCUUGG |
| | CCUUGCUCUACUCUAGGUUAGAAAGUGCUCUCAACCUGUUCACGGUUACAAAGGUGAUAUG |
| | GAAG[spacer] (SEQ ID NO: 408) |
| SEQ ID NO: 17 | AGAAGGGACAGAAACUCAAAGACCAUGAACAGUUUUCGGCGUGGUACGCCGAUCUGAAAAA |
| | CGGGGGUGGAGGCCAUGCAGAUGGUCCUUAGUUACAGCCUCCGUGGACUUGUGGGGUGACU |
| | GUGACA[spacer] (SEQ ID NO: 409) |
| | AGAAGGGACAGAAACUCAAAGACCAUGAACAGUUUUCGGCGUGGUACGCCGAUCUGAAAAA |
| | CGGGGGUGGAGGCCAUAAAGAUGGUCCUUAGUUACAGCCUCCGUGGACUUGUGGGGUGACU |
| | GUGACA[spacer] (SEQ ID NO: 410) |
| | AGAAGGGACAGAAACUCAAAGACCAUGAACAGUUUUCGGCGUGGUACGCCGAUCUGAAAAA |
| | CGGGGGUGGAGGCCAUGCAGAUGGUCCUUAGUUACAGCCUCCGUGGACUGCGUUGCGGGUG |
| | UAUGACCGGAAGUUUCUUUGUGGGGUGACUGUGACA[spacer] (SEQ ID NO: 411) |
| | AGAAGGGACAGAAACUCAAAGACCAUGAACAGUUUUCGGCGUGGUACGCCGAUCUGAAAAA |
| | CGGGGGUGGAGGCCAUGCAGAUGGUCCUUAGUUACAGCCUCCGUGGACUGCGUUGCGGAAA |
| | GCCGGAAGUUUCUUUGUGGGGUGACUGUGACA[spacer] (SEQ ID NO: 412) |
| | AGAAGGGACAGAAACUCAAAGACCAUGAACAGUUUUCGGCGUGGUACGCCGAUCUGAAAAA |
| | CGGGGGUUGUGGGGUGACUGUGACA[spacer] (SEQ ID NO: 413) |
| | AGAAGGGACAGAAACUCAAAGACCAUGAACAGUUUUCGGCGUAAAGACGCCGAUCUGAAAA |
| | ACGGGGGUUGUGGGGUGACUGUGACA[spacer] (SEQ ID NO: 414) |
| SEQ ID NO: 18 | CUACACAGCCGGCGGGACGUAUCCCGUCGGCCGCGCCCGACUGCACGGGCGAGGCAGUGAA |
| | GGCGUCAGACGUCUCUCCACGCGCGCGCGGGAUGCGGG[spacer] (SEQ ID NO: |
| | 415) |
| | CUACACAGCCGGCGGGACGUAUCCCGUCGGCCGCGCCCGACUGCACGGGCGAGGCAGUGAA |
| | AAGUCAGACGUCUCUCCACGCGCGCGCGGGAUGCGGG[spacer] (SEQ ID NO: |
| | 416) |
| | CUACACAGCCGGCGGGACGUAUCCCGUCGGCCGCGCCCGACUGCACUCUCCACGCGCGCGC |
| | GGGAUGCGGG[spacer] (SEQ ID NO: 417) |
| | CUACACAGCCGGCGGGACGUAUCCCGUCGGCCGCGCCCGACUGCACAAAGGCGCGCGCGGG |
| | AUGCGGG[spacer] (SEQ ID NO: 418) |
| SEQ ID NO: 19 | AUGGCAGGCGCUGCCUAGUGCGCCCGUCGCGGUAGUAAUCACGUCGAGACGCAAAACGCCU |
| | GGGGACGGUGUAGGUAGCAAACCGGAUGAACCAGGGAUUCGCGUGGAGUGAUGG[spacer] |
| | (SEQ ID NO: 419) |
| SEQ ID NO: 20 | AUGGCAGGCGCUGCCUAGUGCGCCCGUCGCGGUAGUAAUCACGUCGAGACGCAAAACGCCU |
| | GGGGACGGUGUAGGUAGCAAACCGGAUGAACCAGGAGUUCACCCCACAGGCGCGUGGAGUG |
| | AUGG[spacer] (SEQ ID NO: 420) |
| | AUGGCAGGCGCUGCCUAGUGCGCCCGUCGCGGUAGUAAUCACGUCGAGACGCAAAACGCCU |
| | GGGGACGGUGUAGGUAGCAAACCGGAUGAACAAAGGUUCACCCCACAGGCGCGUGGAGUGA |
| | UGG[spacer] (SEQ ID NO: 421) |
| | GCAGUGCCCGACCUGCGGCGCAGAACAUGAUCGGUUCACCCCACAGGCGCGUGGAGUGAUG |
| | G[spacer] (SEQ ID NO: 422) |
| | GCAGUGCCCGACCUGCGGCGCAGAACAUGAUCGGUUCACCCCACAAAGGUGGAGUGAUGG |
| | [spacer] (SEQ ID NO: 423) |
| | GCAGUGCCCGACCUGCGGCGCAGAACAUGAUCGGGACGGCAACGCAGCGGUCAACAUCCGC |
| | AAGUUCACCCCACAGGCGCGUGGAGUGAUGG[spacer] (SEQ ID NO: 424) |
| | GCAGUGCCCGACCUGCGGCGCAGAACAUGAUCGGGACGGCAACGCAGCGGUCAACAUCCGC |
| | AAGUUCACCCCACAAAGGUGGAGUGAUGG[spacer] (SEQ ID NO: 425) |
| SEQ ID NO: 21 | AUGGCGAAACCACGAACCAAACAAGAGUACGGCCCGUUUAGCGUGCGUCUGCCUGUUGAGA |
| | UGCGCCAGCAGAUCGAAACGUUGGCAGAACAAGAGAUGCGGUCGCUUCACUCGAUGGUUCA |
| | CCCCACGGGUGCGUGGAGUGAUGG[spacer] (SEQ ID NO: 426) |
| | AUGGCGAAACCACGAACCAAACAAGAGUACGGCCCGUUUAGCGUGCGUCUGCCUGUUGAGA |
| | UGCGCCAGCAGAUCGAAACGUUGGCAGAACAAGAGAUGCGGUCGCUUCACUCGAUGGUUCA |
| | CCCCACGAAAGCGUGGAGUGAUGG[spacer] (SEQ ID NO: 427) |
| SEQ ID NO: 22 | GCAGCGUCGCGGGGUUUUAAUGGGCCGACUUCCCGGCCUGAAGCCGAGGUAGCAGCCAUGU |
| | ACGGCGAUUCUCGAAUCGAGGAAGGAAACAUGGACGCGCUCGGUAAAUCAUGAAUGGAUGC |
| | UGAUUGAUGGAAGG[spacer] (SEQ ID NO: 428) |
| | GCAGCGUCGCGGGGUUUUAAUGGGCCGACUUCCCGGCCUGAAGCCGAGGUAGCAGCCAUGU |
| | ACGGCGAUUCUCGAAAGCGAGGAAGGAAACAUGGACGCGCUCGGUAAAUCAUGAAUGGAUG |
| | CUGAUUGAUGGAAGG[spacer] (SEQ ID NO: 429) |
| | UCGAGGAAGGAAACAUGGACGCGCUCGGUAAAUCGUCCCGAGAUGGGACCAAUCAGCAUCC |
| | GCCUUGCGACGCUGCCAUGAAUGGAUGCUGAUUGAUGGAAGG[spacer] (SEQ ID |
| | NO: 430) |
| | UCGAGGAAGGAAACAUGGACGCGCUCGGUAAAUCGUCCCGAGAUGGGACCAAUCAGCAUCC |
| | GCCUUGCGAAAGUGCCAUGAAUGGAUGCUGAUUGAUGGAAGG[spacer] (SEQ ID |
| | NO: 431) |
| SEQ ID NO: 23 | AAUGGGCCGACUUCCCGGCCUGAAGCCGAGGUAGCAGCCAUGUACGGCGAUUCUCGAAUCG |
| | AGGAAGGAAACAUGGACGCGCUCGGUAAAUCGUCCCGAGAUGGGACCAAUCAGCAUGAAUG |
| | GAUGCUGAUUGAUGGAAGG[spacer] (SEQ ID NO: 432) |
| | AAUGGGCCGACUUCCCGGCCUGAAGCCGAGGUAGCAGCCAUGUACGGCGAUUCUCGAAUCG |
| | AGGAAGGAAACAUGGACGCGCUCGGUAAAUCGUCCCGAGAUGGGACCAAUCAGCAUAAAGA |
| | UGCUGAUUGAUGGAAGG[spacer] (SEQ ID NO: 433) |
| | GCAGCGUCGCGGGGUUUUAAUGGGCCGACUUCCCGGCCUGAAGCCGAGGUAGCAGCCAUGU |
| | ACGGCGAUUCUCGAAUCGAGGAAGGAAACAUGGACGCGCUCGGUAAAUCGUCCCGAGAUGG |
| | GACCAAUCAUGAAUGGAUGCUGAUUGAUGGAAGG[spacer] (SEQ ID NO: 434) |
| | GCAGCGUCGCGGGGUUUUAAUGGGCCGACUUCCCGGCCUGAAGCCGAGGUAGCAGCCAUGU |
| | ACGGCGAUUCUCGAAUCGAGGAAGGAAACAUGGACGCGCUCGGUAAAUCGUCCCAAAGGGG |
| | ACCAAUCAUGAAUGGAUGCUGAUUGAUGGAAGG[spacer] (SEQ ID NO: 435) |
| SEQ ID NO: 24 | GAAACCAUAGGUAGAGGCGCCACCACCUUACAUGGUGCCGAUACCGCUCCGUUGGUGCAGU |
| | GUGGACUGUAAUGGUAGAGGCUUAUGGUAGAGGUGCCACCGGUUUACAUGGCGCCGAUACC |
| | [spacer] (SEQ ID NO: 436) |
| | GAAACCAUAGGUAGAGGCGCCACCACCUUACAUGGUGCCGAUACCGCUCCGUUGGUGCAGU |
| | GUGGACUGUAAUGGUAGAGGCUAAAGAGAGGUGCCACCGGUUUACAUGGCGCCGAUACC |
| | [spacer] (SEQ ID NO: 437) |
| | UUAAUAAUCUGAUUACGGCUGAUUGCCGCCGGUAGAGGUGCCACCGCCUUACAUGACACUG |
| | AUACCUUAUAUCCAGCCGUAUUUAUGGUAGAGGUGCCACCGGUUUACAUGGCGCCGAUACC |
| | [spacer] (SEQ ID NO: 438) |
| | UUAAUAAUCUGAUUACGGCUGAUUGCCGCCGGUAGAGGUGCCACCGCCUUACAUGACACUG |
| | AUACCUUAUAUCCAGCCGUAUUUAUGGUAGAGGUGCCACCGAAAGCAUGGCGCCGAUACC |
| | [spacer] (SEQ ID NO: 439) |
| SEQ ID NO: 25 | AAGAAUACUGAUAUUCUCUAUCGGUCCGAAAAGUUUGAAUUGUUUUGGAAUCGUAGGCCGG |
| | UUUGUGCGCCAACCGCAGAAGAGCUGGCCUUGCUCACAAUCACCAGUGAAAACUUGCGGAC |
| | AGUAUGGAACGAAUGGAACGGCCUCCUCAAAGCCAACCGAGGGGGUAGGCUAC[spacer] |
| | (SEQ ID NO: 440) |
| | AAGAAUACUGAUAUUCUCUAUCGGUCCGAAAAGUUUGAAUUGUUUUGGAAUCGUAGGCCGG |
| | UUUGUGCGCCAACCGCAGAAGAGCUGGCCUUGCUCACAAUCACCAGUGAAAACUUGCGGAC |
| | AGUAUGGAACGAAUGGAACGGCCUCCUCAAAGGAGGGGGUAGGCUAC[spacer] (SEQ |
| | ID NO: 441) |
| | UAUGCUCAUUGAAAACAAGGCAUGAGAAAAAUACCCCCGGUUUUGGGCCGGAAUGGAAUGU |
| | UUUUCUCACUGCCAUACCGUUUUUAUGAGGCAACCCUUUAGGGCACUGCAUUGGGCAGUGU |
| | UUAUGCGCUGUUGGAACGGCCUCCUCAAAGCCAACCGAGGGGGUAGGCUAC[spacer] |
| | (SEQ ID NO: 442) |
| | UAUGCUCAUUGAAAACAAGGCAUGAGAAAAAUACCCCCGGUUUUGGGCCGGAAUGGAAUGU |
| | UUUUCUCACUGCCAUACCGUUUUUAUGAGGCAACCCUUUAGGGCACUGCAUUGGGCAGUGU |
| | UUAUGCGCUGUUGGAACGGCCUCCUCAAAGGAGGGGGUAGGCUAC[spacer] (SEQ ID |
| | NO: 443) |
| SEQ ID NO: 26 | CUUAUCGUGAGGUGCAGCCACGAUGUGCGAAUCUAAAGUGGACAGAAAUGGGAACAGACGU |
| | UCGCACGAAUGUUGGCGGAUUUCUUCGGAAAUCGAGCCAAUCAUCAUAUCCAUAUUAUCUG |
| | CGGAUGGAUGUGAACUGCAAG[spacer] (SEQ ID NO: 444) |
| SEQ ID NO: 27 | CUUAUCGUGAGGUGCAGCCACGAUGUGCGAAUCUAAAGUGAACAGAAAUGGGAACAGACGU |
| | UCGCACGAAUGUUGGCGGAUUUCUUCGGAAAUCGAGCCAAUAAUCAUAUCCAUAUUACCGG |
| | GUUGCUGCGGAUGGAUGUGAACUGCAAG[spacer] (SEQ ID NO: 445) |
| SEQ ID NO: 28 | GUGAGGUGCAGCCACGAGGUGCGAAUAGGAAGUACGCAGCAAUGUGCUGAAUCGUUCGCAC |
| | GAAAAUUGGCAUUUUUGAAAACCAAAGCCAAUAAUCAUAUCCAUAAAUAUGUGUGGUUAUG |
| | AACUGCAAG[spacer] (SEQ ID NO: 446) |
| SEQ ID NO: 29 | CUUAUCGUGAGGUGCAGCCACGAUGUGCGAAUCUAAAGUGAACAGAAAUGGGAACAGACGU |
| | UCGCACGAAUGUUGGCGGAUUUCUUCGGAAAUCGAGCCAAUAAUCAUAUCCAUAUUACCGG |
| | GUUGCUCUAUAGAUAGUGUUGGGUACACUUGCUGAACACACUGCGGAUGGAUGUGAACUGC |
| | AAG[spacer] (SEQ ID NO: 447) |
| | CUUAUCGUGAGGUGCAGCCACGAUGUGCGAAUCUAAAGUGAACAGAAAUGGGAACAGACGU |
| | UCGCACGAAUGUUGGCGGAUUUCUUCGGAAAUCGAGCCAAUAAUCAUAUCCAUAUUACCGG |
| | GUUGCUCUAUAGAUAGUGUAAAGACACUUGCUGAACACACUGCGGAUGGAUGUGAACUGCA |
| | AG[spacer] (SEQ ID NO: 448) |
| | CUUAUCGUGAGGUGCAGCCACGAUGUGCGAAUCUAAAGUGAACAGAAAUGGGAACAGACGU |
| | UCGCACGAAUGUUGGCGGAUUUCUUCGGAAAUCGAGCCAAUAAUCAUAUCCAUAUUACCGG |
| | GUUGCUCUAUAGAUAGUGUUGGGUACACUUGCUGAACACAAGAUAUUGCCUGCGGAUGGAU |
| | GUGAACUGCAAG[spacer] (SEQ ID NO: 449) |
| | CUUAUCGUGAGGUGCAGCCACGAUGUGCGAAUCUAAAGUGAACAGAAAUGGGAACAGACGU |
| | UCGCACGAAUGUUGGCGGAUUUCUUCGGAAAUCGAGCCAAUAAUCAUAUCCAUAUUACCGG |
| | GUUGCUCUAUAGAUAGUGUUGGGUAAAAGUGCUGAACACAAGAUAUUGCCUGCGGAUGGAU |
| | GUGAACUGCAAG[spacer] (SEQ ID NO: 450) |
| | CUUAUCGUGAGGUGCAGCCACGAUGUGCGAAUCUAAAGUGAACAGAAAUGGGAACAGACGU |
| | UCGCACGAAUGUUGGCGGAUUUCUUCGGAAAUCGAGCCAAUAAUCAUAUCCAUAUUACCGG |
| | GUUGCUCUCUGCGGAUGGAUGUGAACUGCAAG spacer] (SEQ ID NO: 451) |
| | CUUAUCGUGAGGUGCAGCCACGAUGUGCGAAUCUAAAGUGAACAGAAAUGGGAACAGACGU |
| | UCGCACGAAUGUUGGCGGAUUUCUUCGGAAAUCGAGCCAAUAAUCAUAUCCAUAUUACCGG |
| | GAAAGUGCGGAUGGAUGUGAACUGCAAG[spacer] (SEQ ID NO: 452) |
| SEQ ID NO: 30 | UACCGCAGUUUGGGCGGGUUGCUAAUAAUAUUAGCUUAUGACUUCUAAGGCUUUUGCCUAA |
| | AAAGUAAGGGGGAAGGCGACAACCCCCAAAGACCAGCCGGAACUAUGGCUGGCAACUAUCU |
| | CAUCUUUUUGGCAUAUCAAAGCUAGGGCAAAAACCCCAGACAUUCGCCAAAAGCCCAGAAC |
| | CAUGACAUUGCAAGAGUUUCGCCCAGUUUCUUUUAAAGACCCAAGCUGAUUUAAGCGGCUG |
| | AAAUGAGAUCUUGCAACUGGGCUUGGGGACUGAGGAUAGUUGAAAC[spacer] (SEQ |
| | ID NO: 453) |
| | UACCGCAGUUUGGGCGGGUUGCUAAUAAUAUUAGCUUAUGACUUCUAAGGCUUUUGCCUAA |
| | AAAGUAAGGGGGAAGGCGACAACCCCCAAAGACCAGCCGGAACUAUGGCUGGCAACUAUCU |
| | CAUCUUUUUGGCAUAUCAAAGCUAGGGCAAAAACCCCAGACAUUCGCCAAAAGCCCAGAAC |
| | CAUGACAUUGCAAGAGUUUCGCCCAGUUUCUUAAAGAAGACCCAAGCUGAUUUAAGCGGCU |
| | GAAAUGAGAUCUUGCAACUGGGCUUGGGGACUGAGGAUAGUUGAAAC[spacer] (SEQ |
| | ID NO: 454) |
| | UACCGCAGUUUGGGCGGGUUGCUAAUAAUAUUAGCUUAUGACUUCUAAGGCUUUUGCCUAA |
| | AAAGUAAGGGGGAAGGCGACAACCCCCAAAGACCAGCCGGAACUAUGGCUGGCAACUAUCU |
| | CAUCUUUUUGGCAUAUCAAAGCUAGGGCAAAAACCCCAGACAUUCGCCAAAAGCCCAGAAC |
| | CAUGACAUUGCAAGAGUUUCGCCCAGUUUCUUUUAAAGACCCAAGCUGAUUUAAGCGGCUG |
| | AAAUGAGAUUUUUUAACAGGUCCGCCAAAAUCGCUUCUGCUUGCAACUGGGCUUGGGGACU |
| | GAGGAUAGUUGAAAC[spacer] (SEQ ID NO: 455) |
| | UACCGCAGUUUGGGCGGGUUGCUAAUAAUAUUAGCUUAUGACUUCUAAGGCUUUUGCCUAA |
| | AAAGUAAGGGGGAAGGCGACAACCCCCAAAGACCAGCCGGAACUAUGGCUGGCAACUAUCU |
| | CAUCUUUUUGGCAUAUCAAAGCUAGGGCAAAAACCCCAGACAUUCGCCAAAAGCCCAGAAC |
| | CAUGACAUUGCAAGAGUUUCGCCCAGUUUCUUUUAAAGACCCAAGCUGAUUUAAGCGGCUG |
| | AAAUGAGAAAGUUUAACAGGUCCGCCAAAAUCGCUUCUGCUUGCAACUGGGCUUGGGGACU |
| | GAGGAUAGUUGAAAC[spacer] (SEQ ID NO: 456) |
| | AUAAGAUGGACUAUAUUUAUAAACCAGGAAACAUAGUUAUUACAAUUGCAUUGUAGGUUUC |
| | AACCUGAUACCCACUAAAGCGUGUUAGUGGGUAUUUUUUCUUGCAACUGGGCUUGGGGACU |
| | GAGGAUAGUUGAAAC[spacer] (SEQ ID NO: 457) |
| | AUAAGAUGGACUAUAUUUAUAAACCAGGAAACAUAGUUAUUACAAUUGCAUUGUAGGUUUC |
| | AACCUGAUACCCACUAAAAAGUUAGUGGGUAUUUUUUCUUGCAACUGGGCUUGGGGACUGA |
| | GGAUAGUUGAAAC[spacer] (SEQ ID NO: 458) |
| | CUUGACAGUAAACAAAAAAUAAGAUGGACUAUAUUUAUAAACCAGGAAACAUAGUUAUUAC |
| | AAUUGCAUUGUAGGUUUCAACCUGAUACCCACUAAAGCGUGUUAGUGGGUAUUUUUUAUCU |
| | UGCAACUGGGCUUGGGGACUGAGGAUAGUUGAAAC[spacer] (SEQ ID NO: 459) |
| | CUUGACAGUAAACAAAAAAUAAGAUGGACUAUAUUUAUAAACCAGGAAACAUAGUUAUUAC |
| | AAUUGCAUUGUAGGUUUCAACCUGAUACCCACUAAAGCGUGUUAGUGGGUAUUUUUUAUCU |
| | UGCAACUGGGCUUGGAAAGCUGAGGAUAGUUGAAAC[spacer] (SEQ ID NO: 460) |
| SEQ ID NO: 31 | AGCCGCACGGAACCUGAGCCGAUGGCGUAGCCCUUGGACCUAUAUGGAACGCGGCAUAAGC |
| | CCUGCGAGUUCGCAAGAGCCCAAGGCGGCAUGACAAGCCUCUUUCAGGCGACAGAGUCUUU |
| | UGGAGUGUCGAGGCUCCCUGCAUUCCUUGGGAGCCUCCCAAAGGAAUGAAAG[spacer] |
| | (SEQ ID NO: 461) |
| | AGCCGCACGGAACCUGAGCCGAUGGCGUAGCCCUUGGACCUAUAUGGAACGCGGCAUAAGC |
| | CCUGCGAGUUCGCAAGAGCCCAAGGCGGCAUGACAAGCCUCUUUCAGGCGACAGAGUCUUU |
| | UGGAGUGUCGAGGCUCCCAAAGGGGAGCCUCCCAAAGGAAUGAAAG [spacer] (SEQ |
| | ID NO: 462) |
| | AGCCGCACGGAACCUGAGCCGAUGGCGUAGCCCUUGGACCUAUAUGGAACGCGGCAUAAGC |
| | CCUGCGAGUUCGCAAGAGCCCAAGGCGGCAUGAAAGGAAUGAAAG[spacer] (SEQ ID |
| | NO: 463) |
| | AGCCGCACGGAACCUGAGCCGAUGGCGUAGCCCUUGGACCUAUAUGGAACGCGGCAUAAGC |
| | CCUGCGAGUUCAAAGGAGCCCAAGGCGGCAUGAAAGGAAUGAAAG[spacer] (SEQ ID |
| | NO: 464) |
| SEQ ID NO: 32 | GUUCCGUCUCGACUAUGCCGUACCACUAGACCGAGCCUACACGGCACGCGGUCAUAGCGUU |
| | AACCAAGGCGUGGUGACAAGCCUCUUUCAGGCGUCGGACACUUAAGAGCGUUAGGCGGGCG |
| | GUCCCUAAGCCGCCCGCCCCCACGCUCUUAGGGAAUGAAAG[spacer] (SEQ ID NO: |
| | 465) |
| | GUUCCGUCUCGACUAUGCCGUACCACUAGACCGAGCCUACACGGCACGCGGUCAUAGCGUU |
| | AACCAAGGCGUGGUGACAAGCCUCUUUCAGGCGUCGGACACUUAAGAGCGUUAGGCGGGCG |
| | GUAAAGGCCGCCCGCCCCCACGCUCUUAGGGAAUGAAAG[spacer] (SEQ ID NO: |
| | 466) |
| | GUUCCGUCUCGACUAUGCCGUACCACUAGACCGAGCCUACACGGCACGCGGUCAUAGCGUU |
| | AACCAAGGCGUGGUGACAAGCCUCUUUCAGGCGUCGGACACUUAAGAGCGUUAGGCGGGCG |
| | GUCCCUAAGCCGCCCGCCCCCUUAUUUGCACGUUUUCCCCGAACCCCGUAAACGCUCUUAG |
| | GGAAUGAAAG[spacer] (SEQ ID NO: 467) |
| | GUUCCGUCUCGACUAUGCCGUACCACUAGACCGAGCCUACACGGCACGCGGUCAUAGCGUU |
| | AACCAAGGCGUGGUGACAAGCCUCUUUCAGGCGUCGGACACUUAAGAGCGUUAGGCGGGCG |
| | GUCCCUAAGCCGCCCGCCCCCUUAUUUGCACGUUUAAAGGAACCCCGUAAACGCUCUUAGG |
| | GAAUGAAAG[spacer] (SEQ ID NO: 468) |
| | GUUCCGUCUCGACUAUGCCGUACCACUAGACCGAGCCUACACGGCACGCGGUCAUAGCGUU |
| | AACCAAGGCGUGGUGACAAGCCUCUUUCAGGCGUCGGACACUUAAGAGCGUUAACGCUCUU |
| | AGGGAAUGAAAGGUUCCGUCUCGACUAUGCCGUACCACUAGACCGAGCCUACACGGCACGC |
| | GGUCAUAGCGUUAACCAAGGCGUGGUGACAAGCCUCUUUCAGGCGUCGGACACUUAAGAGC |
| | GAAAGCGCUCUUAGGGAAUGAAAG[spacer] (SEQ ID NO: 469) |

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 1, and the tracrRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 129 or a portion of the nucleotide sequence of SEQ ID NOs: 129. In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 1, and the sgRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 300 or a portion of the nucleotide sequence of SEQ ID NO: 300.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 2, and the tracrRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of any one of SEQ ID NOs: 130-137 or a portion of the nucleotide sequence of any one of SEQ ID NOs: 130-137. In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 2, and the sgRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of any one of SEQ ID NOs: 301-308 or a portion of the nucleotide sequence of any one of SEQ ID NOs: 301-308. In other embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 2, and the sgRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of any one of SEQ ID NOs: 305-308 or a portion of the nucleotide sequence of any one of SEQ ID NOs: 305-308.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 3, and the tracrRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of any one of SEQ ID NOs: 138-147 or a portion of the nucleotide sequence of any one of SEQ ID NOs: 138-147. In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 3, and the sgRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of any one of SEQ ID NOs: 309-318 or a portion of the nucleotide sequence of any one of SEQ ID NOs: 309-318. In other embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 3, and the sgRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of any one of SEQ ID NOs: 310-312 or a portion of the nucleotide sequence of any one of SEQ ID NOs: 310-312.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 4, and the tracrRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of any one of SEQ ID NOs: 148-149 or a portion of the nucleotide sequence of any one of SEQ ID NOs: 148-149. In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 4, and the sgRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of any one of SEQ ID NOs: 319-320 or a portion of the nucleotide sequence of any one of SEQ ID NOs: 319-320.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 5, and the tracrRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of any one of SEQ ID NOs: 150-161 or a portion of the nucleotide sequence of any one of SEQ ID NOs: 150-161. In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 5, and the sgRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of any one of SEQ ID NOs: 321-334 or a portion of the nucleotide sequence of any one of SEQ ID NOs: 321-334.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 6, and the tracrRNA sequence 89%, 90%, 91%, 92%, 93%, 94%, 95%, 96%, 97%, 98%, 99% or 100%) identical to the nucleotide sequence of any one of SEQ ID NOs: 164-169 or a portion of the nucleotide sequence of any one of SEQ ID NOs: 164-169. In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 6, and the sgRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of any one of SEQ ID NOs: 335-342 or a portion of the nucleotide sequence of any one of SEQ ID NOs: 335-342.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 7, and the tracrRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of any one of SEQ ID NOs: 170-175 or a portion of the nucleotide sequence of any one of SEQ ID NOs: 170-175. In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 7, and the sgRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of any one of SEQ ID NOs: 341-346 or a portion of the nucleotide sequence of any one of SEQ ID NOs: 341-346.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 8, and the tracrRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of any one of SEQ ID NOs: 176-177 or a portion of the nucleotide sequence of any one of SEQ ID NOs: 176-177. In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 8, and the sgRNA sequence sequence of any one of SEQ ID NOs: 347-348 or a portion of the nucleotide sequence of any one of SEQ ID NOs: 347-348.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 9, and the tracrRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of any one of SEQ ID NOs: 178-183 or a portion of the nucleotide sequence of any one of SEQ ID NOs: 178-183. In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 9, and the sgRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of any one of SEQ ID NOs: 349-354 or a portion of the nucleotide sequence of any one of SEQ ID NOs: 349-354.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 10, and the tracrRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of any one of SEQ ID NOs: 184-193 or a portion of the nucleotide sequence of any one of SEQ ID NOs: 184-193. In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 10, and the sgRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of any one of SEQ ID NOs: 355-364 or a portion of the nucleotide sequence of any one of SEQ ID NOs: 355-364.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 11, and the tracrRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of any one of SEQ ID NOs: 194-199 or a portion of the nucleotide sequence of any one of SEQ ID NOs: 194-199. In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 11, and the sgRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of any one of SEQ ID NOs: 365-370 or a portion of the nucleotide sequence of any one of SEQ ID NOs: 365-370.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 12, and the tracrRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of any one of SEQ ID NOs: 200-211 or a portion of the nucleotide sequence of any one of SEQ ID NOs: 200-211. In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 12, and the sgRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of any one of SEQ ID NOs: 371-382 or a portion of the nucleotide sequence of any one of SEQ ID NOs: 371-382.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 13, and the tracrRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of any one of SEQ ID NOs: 212-219 or a portion of the nucleotide sequence of any one of SEQ ID NOs: 212-219. In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 13, and the sgRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of any one of SEQ ID NOs: 383-390 or a portion of the nucleotide sequence of any one of SEQ ID NOs 383-390.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 14, and the tracrRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of any one of SEQ ID NOs: 220-227 or a portion of the nucleotide sequence of any one of SEQ ID NOs: 220-227. In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 14, and the sgRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of any one of SEQ ID NOs: 391-398 or a portion of the nucleotide sequence of any one of SEQ ID NOs: 391-398.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 15, and the tracrRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of any one of SEQ ID NOs: 228-233 or a portion of the nucleotide sequence of any one of SEQ ID NOs: 228-233. In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 15, and the sgRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of any one of SEQ ID NOs: 399-404 or a portion of the nucleotide sequence of any one of SEQ ID NOs: 399-404.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 16, and the tracrRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of any one of SEQ ID NOs: 234-237 or a portion of the nucleotide sequence of any one of SEQ ID NOs: 243-237. In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 16, and the sgRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of any one of SEQ ID NOs: 405-408 or a portion of the nucleotide sequence of any one of SEQ ID NOs: 405-408.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 17, and the tracrRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of any one of SEQ ID NOs: 238-243 or a portion of the nucleotide sequence of any one of SEQ ID NOs: 238-243. In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 17, and the sgRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of any one of SEQ ID NOs: 409-414 or a portion of the nucleotide sequence of any one of SEQ ID NOs: 409-414.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 18, and the tracrRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of any one of SEQ ID NOs: 244-247 or a portion of the nucleotide sequence of any one of SEQ ID NOs: 244-247. In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 18, and the sgRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of any one of SEQ ID NOs: 415-418 or a portion of the nucleotide sequence of any one of SEQ ID NOs: 415-418. In other embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 18, and the sgRNA sequence 89%, 90%, 91%, 92%, 93%, 94%, 95%, 96%, 97%, 98%, 99% or 100%) identical to the nucleotide sequence of SEQ ID NO: 416 or a portion of the nucleotide sequence of SEQ ID NO: 416.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 19, and the tracrRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 248 or a portion of the nucleotide sequence of SEQ ID NO: 248. In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 19, and the sgRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 419 or a portion of the nucleotide sequence of SEQ ID NO: 419.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 20, and the tracrRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of any one of SEQ ID NOs: 249-254 or a portion of the nucleotide sequence of any one of SEQ ID NOs: 249-254. In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 20, and the sgRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of any one of SEQ ID NOs: 420-425 or a portion of the nucleotide sequence of any one of SEQ ID NOs: 420-425. In other embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 20, and the sgRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 420 or a portion of the nucleotide sequence of SEQ ID NO: 420.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 21, and the tracrRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of any one of SEQ ID NOs: 255-256 or a portion of the nucleotide sequence of any one of SEQ ID NOs: 255-256. In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 21, and the sgRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of any one of SEQ ID NOs: 426-427 or a portion of the nucleotide sequence of any one of SEQ ID NOs: 426-427.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 22, and the tracrRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of any one of SEQ ID NOs: 257-260 or a portion of the nucleotide sequence of any one of SEQ ID NOs: 257-260. In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 22, and the sgRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of any one of SEQ ID NOs: 428-431 or a portion of the nucleotide sequence of any one of SEQ ID NOs: 428-431. In other embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 22, and the sgRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 428 or 429 or a portion of the nucleotide sequence of SEQ ID NO: 428 or 429.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 23, and the tracrRNA sequence sequence of any one of SEQ ID NOs: 261-264 or a portion of the nucleotide sequence of any one of SEQ ID NOs: 261-264. In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 23, and the sgRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of any one of SEQ ID NOs: 432-435 or a portion of the nucleotide sequence of any one of SEQ ID NOs: 432-435.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 24, and the tracrRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of any one of SEQ ID NOs: 265-268 or a portion of the nucleotide sequence of any one of SEQ ID NOs: 265-268. In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 24, and the sgRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of any one of SEQ ID NOs: 436-439 or a portion of the nucleotide sequence of any one of SEQ ID NOs: 436-439.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 25, and the tracrRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of any one of SEQ ID NOs: 269-272 or a portion of the nucleotide sequence of any one of SEQ ID NOs: 269-272. In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 25, and the sgRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of any one of SEQ ID NOs: 440-443 or a portion of the nucleotide sequence of any one of SEQ

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 26, and the tracrRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 273 or a portion of the nucleotide sequence of SEQ ID NO: 273. In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 26, and the sgRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 444 or a portion of the nucleotide sequence of SEQ ID NO: 444.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 27, and the tracrRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 274 or a portion of the nucleotide sequence of SEQ ID NO: 274. In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 27, and the sgRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 445 or a portion of the nucleotide sequence of SEQ ID NO: 445.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 28, and the tracrRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 275 or a portion of the nucleotide sequence of SEQ ID NO: 275. In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 28, and the sgRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 446 or a portion of the nucleotide sequence of SEQ ID NO: 446.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 29, and the tracrRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of any one of SEQ ID NOs: 276-281 or a portion of the nucleotide sequence of any one of SEQ ID NOs: 276-281. In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 29, and the sgRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of any one of SEQ ID NOs: 447-452 or a portion of the nucleotide sequence of any one of SEQ ID NOs: 447-452.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 30, and the tracrRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of any one of SEQ ID NOs: 282-289 or a portion of the nucleotide sequence of any one of SEQ ID NOs: 282-289. In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 30, and the sgRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of any one of SEQ ID NOs: 453-460 or a portion of the nucleotide sequence of any one of SEQ ID NOs: 453-460.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 31, and the tracrRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of any one of SEQ ID NOs: 290-293 or a portion of the nucleotide sequence of any one of SEQ ID NOs: 290-293. In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 31, and the sgRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of any one of SEQ ID NOs: 461-464 or a portion of the nucleotide sequence of any one of SEQ ID NOs: 461-464.

In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 32, and the tracrRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of any one of SEQ ID NOs: 294-299 or a portion of the nucleotide sequence of any one of SEQ ID NOs: 294-299. In some embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 32, and the sgRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of any one of SEQ ID NOs: 465-469 or a portion of the nucleotide sequence of any one of SEQ ID NOs: 465-469. In other embodiments, the nuclease comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 32, and the sgRNA sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 469 or a portion of the nucleotide sequence of SEQ ID NO: 469.

In some embodiments, the tracrRNA sequences disclosed herein (SEQ ID NOs: 219-299) are capable of binding to any one or more of the nuclease polypeptides disclosed herein. In some embodiments, the sgRNA sequences disclosed herein (SEQ ID NOs: 300-469) are capable of binding to any one or more of the nuclease polypeptides disclosed herein.

In some embodiments wherein a nuclease of the present invention forms a dimer, the dimer forms a complex with one or more RNA guide sequences. In some embodiments wherein a nuclease of the present invention forms a dimer, the dimer forms a complex with one or more tracrRNA sequences. In some embodiments, the dimer forms a complex with one tracrRNA sequence and one RNA guide sequence. In some embodiments, the dimer forms a complex with one tracrRNA sequence and two RNA guide sequences. In some embodiments, the dimer forms a complex with two tracrRNA sequences and one RNA guide sequence. In some embodiments, the dimer forms a complex with two tracrRNA sequences and two RNA guide sequences. In some embodiments, the dimer forms a complex with one sgRNA sequence. In some embodiments, the dimer forms a complex with two sgRNA sequences.

In some embodiments, a homodimer comprising two identical RuvC domains forms a complex with one tracrRNA sequence and one RNA guide sequence. In some embodiments, a heterodimer comprising two non-identical RuvC domains forms a complex with one tracrRNA sequence and one RNA guide sequence. In some embodiments, a homodimer comprising two identical RuvC domains forms a complex with one tracrRNA sequence and two RNA guide sequences. In some embodiments, a heterodimer comprising two non-identical RuvC domains forms a complex with one tracrRNA sequence and two RNA guide sequences. In some embodiments, a homodimer comprising two identical RuvC domains forms a complex with two tracrRNA sequences and one RNA guide sequence. In some embodiments, a heterodimer comprising two non-identical RuvC domains forms a complex with two tracrRNA sequences and one RNA guide sequence. In some embodiments, a homodimer comprising two identical RuvC domains forms a complex with two tracrRNA sequences and two RNA guide sequences. In some embodiments, a heterodimer comprising two non-identical RuvC domains forms a complex with two tracrRNA sequences and two RNA guide sequences. In some embodiments, a homodimer comprising two identical RuvC domains forms a complex with one sgRNA sequence. In some embodiments, a heterodimer comprising two non-identical RuvC domains forms a complex with one sgRNA sequence. In some embodiments, a homodimer comprising two identical RuvC domains forms a complex with two sgRNA sequences. In some embodiments, a heterodimer comprising two non-identical RuvC domains forms a complex with two sgRNA sequences.

Unless otherwise noted, all gene editing systems and nucleases provided herein are made in reference to the active level of that gene editing system or nuclease, and are exclusive of impurities, for example, residual solvents or by-products, which may be present in commercially available sources. Nuclease component weights are based on total active protein. All percentages and ratios are calculated by weight unless otherwise indicated. All percentages and ratios are calculated based on the total gene editing system unless otherwise indicated. In the exemplified gene editing system, the nuclease levels are expressed by pure enzyme by weight of the total gene editing system and unless otherwise specified, the ingredients are expressed by weight of the total gene editing systems.

Modifications

The RNA guide sequence, tracrRNA sequence, sgRNA sequence, or any of the nucleic acid sequences encoding a nuclease may include one or more covalent modifications with respect to a reference sequence, in particular the parent polyribonucleotide, which are included within the scope of this invention.

Exemplary modifications can include any modification to the sugar, the nucleobase, the internucleoside linkage (e.g. to a linking phosphate/to a phosphodiester linkage/to the phosphodiester backbone), and any combination thereof. Some of the exemplary modifications provided herein are described in detail below.

The RNA guide sequence, tracrRNA sequence, sgRNA sequence, or any of the nucleic acid sequences encoding components of a nuclease may include any useful modification, such as to the sugar, the nucleobase, or the internucleoside linkage (e.g. to a linking phosphate/to a phosphodiester linkage/to the phosphodiester backbone). One or more atoms of a pyrimidine nucleobase may be replaced or substituted with optionally substituted amino, optionally substituted thiol, optionally substituted alkyl (e.g., methyl or ethyl), or halo (e.g., chloro or fluoro). In certain embodiments, modifications (e.g., one or more modifications) are present in each of the sugar and the internucleoside linkage. Modifications may be modifications of ribonucleic acids (RNAs) to deoxyribonucleic acids (DNAs), threose nucleic acids (TNAs), glycol nucleic acids (GNAs), peptide nucleic acids (PNAs), locked nucleic acids (LNAs) or hybrids thereof). Additional modifications are described herein.

In some embodiments, the modification may include a chemical or cellular induced modification. For example, some nonlimiting examples of intracellular RNA modifications are described by Lewis and Pan in “RNA modifications and structures cooperate to guide RNA-protein interactions” from Nat Reviews Mol Cell Biol, 2017, 18:202-210.

Different sugar modifications, nucleotide modifications, and/or internucleoside linkages (e.g., backbone structures) may exist at various positions in the sequence. One of ordinary skill in the art will appreciate that the nucleotide analogs or other modification(s) may be located at any position(s) of the sequence, such that the function of the sequence is not substantially decreased. The sequence may include from about 1% to about 100% modified nucleotides (either in relation to overall nucleotide content, or in relation to one or more types of nucleotide, i.e. any one or more of A, G, U or C) or any intervening percentage (e.g., from 1% to 20%>, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%).

In some embodiments, sugar modifications (e.g., at the 2′ position or 4′ position) or replacement of the sugar at one or more ribonucleotides of the sequence may, as well as backbone modifications, include modification or replacement of the phosphodiester linkages. Specific examples of a sequence include, but are not limited to, sequences including modified backbones or no natural internucleoside linkages such as internucleoside modifications, including modification or replacement of the phosphodiester linkages. Sequences having modified backbones include, among others, those that do not have a phosphorus atom in the backbone. For the purposes of this application, and as sometimes referenced in the art, modified RNAs that do not have a phosphorus atom in their internucleoside backbone can also be considered to be oligonucleosides. In particular embodiments, a sequence will include ribonucleotides with a phosphorus atom in its internucleoside backbone.

Modified sequence backbones may include, for example, phosphorothioates, chiral phosphorothioates, phosphorodithioates, phosphotriesters, aminoalkylphosphotriesters, methyl and other alkyl phosphonates such as 3′-alkylene phosphonates and chiral phosphonates, phosphinates, phosphoramidates such as 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included. In some embodiments, the sequence may be negatively or positively charged.

The modified nucleotides, which may be incorporated into the sequence, can be modified on the internucleoside linkage (e.g., phosphate backbone). Herein, in the context of the polynucleotide backbone, the phrases “phosphate” and “phosphodiester” are used interchangeably. Backbone phosphate groups can be modified by replacing one or more of the oxygen atoms with a different substituent. Further, the modified nucleosides and nucleotides can include the wholesale replacement of an unmodified phosphate moiety with another internucleoside linkage as described herein. Examples of modified phosphate groups include, but are not limited to, phosphorothioate, phosphoroselenates, boranophosphates, boranophosphate esters, hydrogen phosphonates, phosphoramidates, phosphorodiamidates, alkyl or aryl phosphonates, and phosphotriesters. Phosphorodithioates have both non-linking oxygens replaced by sulfur. The phosphate linker can also be modified by the replacement of a linking oxygen with nitrogen (bridged phosphoramidates), sulfur (bridged phosphorothioates), and carbon (bridged methylene-phosphonates).

The α-thio substituted phosphate moiety is provided to confer stability to RNA and DNA polymers through the unnatural phosphorothioate backbone linkages. Phosphorothioate DNA and RNA have increased nuclease resistance and subsequently a longer half-life in a cellular environment.

In specific embodiments, a modified nucleoside includes an alpha-thio-nucleoside (e.g., 5′-O-(1-thiophosphate)-adenosine, 5′-O-(1-thiophosphate)-cytidine (a-thio-cytidine), 5′-O-(1-thiophosphate)-guanosine, 5′-O-(1-thiophosphate)-uridine, or 5′-O-(1-thiophosphate)-pseudouridine).

Other internucleoside linkages that may be employed according to the present invention, including internucleoside linkages which do not contain a phosphorous atom, are described herein.

In some embodiments, the sequence may include one or more cytotoxic nucleosides. For example, cytotoxic nucleosides may be incorporated into sequence, such as bifunctional modification. Cytotoxic nucleoside may include, but are not limited to, adenosine arabinoside, 5-azacytidine, 4′-thio-aracytidine, cyclopentenylcytosine, cladribine, clofarabine, cytarabine, cytosine arabinoside, 1-(2-C-cyano-2-deoxy-beta-D-arabino-pentofuranosyl)-cytosine, decitabine, 5-fluorouracil, fludarabine, floxuridine, gemcitabine, a combination of tegafur and uracil, tegafur ((RS)-5-fluoro-1-(tetrahydrofuran-2-yl)pyrimidine-2,4(1H,3H)-dione), troxacitabine, tezacitabine, 2′-deoxy-2′-methylidenecytidine (DMDC), and 6-mercaptopurine. Additional examples include fludarabine phosphate, N4-behenoyl-1-beta-D-arabinofuranosylcytosine, N4-octadecyl-1-beta-D-arabinofuranosylcytosine, N4-palmitoyl-1-(2-C-cyano-2-deoxy-beta-D-arabino-pentofuranosyl) cytosine, and P-4055 (cytarabine 5′-elaidic acid ester).

In some embodiments, the sequence includes one or more post-transcriptional modifications (e.g., capping, cleavage, polyadenylation, splicing, poly-A sequence, methylation, acylation, phosphorylation, methylation of lysine and arginine residues, acetylation, and nitrosylation of thiol groups and tyrosine residues, etc.). The one or more post-transcriptional modifications can be any post-transcriptional modification, such as any of the more than one hundred different nucleoside modifications that have been identified in RNA (Rozenski, J, Crain, P, and McCloskey, J. (1999). The RNA Modification Database: 1999 update. Nucl Acids Res 27: 196-197) In some embodiments, the first isolated nucleic acid comprises messenger RNA (mRNA). In some embodiments, the mRNA comprises at least one nucleoside selected from the group consisting of pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine. In some embodiments, the mRNA comprises at least one nucleoside selected from the group consisting of 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine. In some embodiments, the mRNA comprises at least one nucleoside selected from the group consisting of 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine. In some embodiments, mRNA comprises at least one nucleoside selected from the group consisting of inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

The sequence may or may not be uniformly modified along the entire length of the molecule. For example, one or more or all types of nucleotide (e.g., naturally-occurring nucleotides, purine or pyrimidine, or any one or more or all of A, G, U, C, I, pU) may or may not be uniformly modified in the sequence, or in a given predetermined sequence region thereof. In some embodiments, the sequence includes a pseudouridine. In some embodiments, the sequence includes an inosine, which may aid in the immune system characterizing the sequence as endogenous versus viral RNAs. The incorporation of inosine may also mediate improved RNA stability/reduced degradation. See for example, Yu, Z. et al. (2015) RNA editing by ADAR1 marks dsRNA as “self”. Cell Res. 25, 1283-1284, which is incorporated by reference in its entirety.

### II. Preparation of Gene Editing System Components

The present disclosure provides methods for production of components of the gene editing systems disclosed herein, e.g., the RNA guide, methods for production of the nuclease polypeptide, and methods for complexing the RNA guide and nuclease polypeptide.

A. Nuclease Polypeptide

In some embodiments, a nuclease of the present invention can be prepared by (I) culturing bacteria which produce a nuclease of the present invention, isolating the nuclease, and optionally, purifying the nuclease. The nuclease can be also prepared by (II) a known genetic engineering technique, specifically, by isolating a gene encoding a nuclease of the present invention from bacteria, constructing a recombinant expression vector, and then transferring the vector into an appropriate host cell for expression of a recombinant protein. Alternatively, a nuclease can be prepared by (III) an in vitro coupled transcription-translation system. Bacteria that can be used for preparation of a nuclease of the present invention are not particularly limited as long as they can produce a nuclease of the present invention. Some non-limiting examples of the bacteria include E. coli cells described herein.

In some embodiments, a host cell described herein is used to express a nuclease. The host cell is not particularly limited, and various known cells can be preferably used. Specific examples of the host cell include bacteria such as E. coli, yeasts (budding yeast, Saccharomyces cerevisiae, and fission yeast, Schizosaccharomyces pombe), nematodes (Caenorhabditis elegans), Xenopus laevis oocytes, and animal cells (for example, CHO cells, COS cells and HEK293 cells). The method for transferring the expression vector described above into host cells, i.e., the transformation method, is not particularly limited, and known methods such as electroporation, the calcium phosphate method, the liposome method and the DEAE dextran method can be used.

After a host is transformed with the expression vector, the host cells may be cultured, cultivated or bred, for production of a nuclease. After expression of the nuclease, the host cells can be collected and nuclease purified from the cultures etc. according to conventional methods (for example, filtration, centrifugation, cell disruption, gel filtration chromatography, ion exchange chromatography, etc.).

In some embodiments, the methods for nuclease expression comprises translation of at least 5 amino acids, at least 10 amino acids, at least 15 amino acids, at least 20 amino acids, at least 50 amino acids, at least 100 amino acids, at least 150 amino acids, at least 200 amino acids, at least 250 amino acids, at least 300 amino acids, at least 400 amino acids, at least 500 amino acids, at least 600 amino acids, at least 700 amino acids, at least 800 amino acids, at least 900 amino acids, or at least 1000 amino acids of a nuclease. In some embodiments, the methods for protein expression comprises translation of about 5 amino acids, about 10 amino acids, about 15 amino acids, about 20 amino acids, about 50 amino acids, about 100 amino acids, about 150 amino acids, about 200 amino acids, about 250 amino acids, about 300 amino acids, about 400 amino acids, about 500 amino acids, about 600 amino acids, about 700 amino acids, about 800 amino acids, about 900 amino acids, about 1000 amino acids or more of a nuclease.

A variety of methods can be used to determine the level of production of a mature nuclease in a host cell. Such methods include, but are not limited to, for example, methods that utilize either polyclonal or monoclonal antibodies specific for a nuclease. Exemplary methods include, but are not limited to, enzyme-linked immunosorbent assays (ELISA), radioimmunoassays (MA), fluorescent immunoassays (FIA), and fluorescent activated cell sorting (FACS). These and other assays are well known in the art (See, e.g., Maddox et al., J. Exp. Med. 158:1211 [1983]).

The present disclosure provides methods of in vivo expression of the nuclease polypeptide in a cell, comprising providing a polyribonucleotide encoding the nuclease polypeptide to a host cell wherein the polyribonucleotide encodes the nuclease polypeptide, expressing the nuclease polypeptide in the cell, and obtaining the nuclease polypeptide from the cell.

The present disclosure further provides methods of in vivo expression of a nuclease polypeptide in a cell, comprising providing a polyribonucleotide encoding the nuclease polypeptide to a host cell wherein the polyribonucleotide encodes the nuclease polypeptide and expressing the nuclease polypeptide in the cell. In some embodiments, the polyribonucleotide encoding the nuclease polypeptide is delivered to the cell with an RNA guide and, once expressed in the cell, the nuclease polypeptide and the RNA guide form a complex. In some embodiments, the polyribonucleotide encoding the nuclease polypeptide and the RNA guide are delivered to the cell within a single composition. In some embodiments, the polyribonucleotide encoding the nuclease polypeptide and the RNA guide are comprised within separate compositions. In some embodiments, the host cell is present in a subject, e.g., a human patient.

Vectors

The present invention provides a vector for expressing a nuclease described herein or nucleic acids encoding a nuclease described herein may be incorporated into a vector. In some embodiments, a vector of the invention includes a nucleotide sequence encoding a nuclease described herein. In some embodiments, a vector of the invention includes a nucleotide sequence encoding a nuclease described herein.

The present invention also provides a vector that may be used for preparation of a nuclease described herein or gene editing systems comprising a nuclease described herein. In some embodiments, the invention includes the gene editing system or vector described herein in a cell. In some embodiments, the invention includes a method of expressing the gene editing system comprising a nuclease of the present invention, or vector or nucleic acid encoding the nuclease, in a cell. The method may comprise the steps of providing the gene editing system, e.g., vector or nucleic acid, and delivering the gene editing system to the cell.

Expression of natural or synthetic polynucleotides is typically achieved by operably linking a polynucleotide encoding the gene of interest, e.g., nucleotide sequence encoding a nuclease of the present invention, to a promoter and incorporating the construct into an expression vector. The expression vector is not particularly limited as long as it includes a polynucleotide encoding a nuclease of the present invention and can be suitable for replication and integration in eukaryotic cells.

Typical expression vectors include transcription and translation terminators, initiation sequences, and promoters useful for expression of the desired polynucleotide. For example, plasmid vectors carrying a recognition sequence for RNA polymerase (pSP64, pBluescript, etc.). may be used. Vectors including those derived from retroviruses such as lentivirus are suitable tools to achieve long-term gene transfer since they allow long-term, stable integration of a transgene and its propagation in daughter cells. Examples of vectors include expression vectors, replication vectors, probe generation vectors, and sequencing vectors. The expression vector may be provided to a cell in the form of a viral vector.

Viral vector technology is well known in the art and described in a variety of virology and molecular biology manuals. Viruses which are useful as vectors include, but are not limited to phage viruses, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers.

The kind of the vector is not particularly limited, and a vector that can be expressed in host cells can be appropriately selected. To be more specific, depending on the kind of the host cell, a promoter sequence to ensure the expression of a nuclease of the present invention from a polynucleotide is appropriately selected, and this promoter sequence and the polynucleotide are inserted into any of various plasmids etc. for preparation of the expression vector.

Additional promoter elements, e.g., enhancing sequences, regulate the frequency of transcriptional initiation. Typically, these are located in the region 30-110 bp upstream of the start site, although a number of promoters have recently been shown to contain functional elements downstream of the start site as well. Depending on the promoter, it appears that individual elements can function either cooperatively or independently to activate transcription.

Further, the disclosure should not be limited to the use of constitutive promoters. Inducible promoters are also contemplated as part of the disclosure. The use of an inducible promoter provides a molecular switch capable of turning on expression of the polynucleotide sequence which it is operatively linked when such expression is desired or turning off the expression when expression is not desired. Examples of inducible promoters include, but are not limited to a metallothionine promoter, a glucocorticoid promoter, a progesterone promoter, and a tetracycline promoter.

The expression vector to be introduced can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate transcriptional control sequences to enable expression in the host cells. Examples of such a marker include a dihydrofolate reductase gene and a neomycin resistance gene for eukaryotic cell culture; and a tetracycline resistance gene and an ampicillin resistance gene for culture of E. coli and other bacteria. By use of such a selection marker, it can be confirmed whether the polynucleotide encoding a nuclease of the present invention has been transferred into the host cells and then expressed without fail.

The preparation method for recombinant expression vectors is not particularly limited, and examples thereof include methods using a plasmid, a phage or a cosmid.

B. RNA Guide

In some embodiments, the RNA guide is made by in vitro transcription of a DNA template. Thus, for example, in some embodiments, the RNA guide is generated by in vitro transcription of a DNA template encoding the RNA guide using an upstream promoter sequence (e.g., a T7 polymerase promoter sequence).

In some embodiments, the DNA template encodes multiple RNA guides or the in vitro transcription reaction includes multiple different DNA templates, each encoding a different RNA guide. In some embodiments, the RNA guide is made using chemical synthetic methods. In some embodiments, the RNA guide is made by expressing the RNA guide sequence in cells transfected with a plasmid including sequences that encode the RNA guide. In some embodiments, the plasmid encodes multiple different RNA guides. In some embodiments, multiple different plasmids, each encoding a different RNA guide, are transfected into the cells. In some embodiments, the RNA guide is expressed from a plasmid that encodes the RNA guide and also encodes a nuclease polypeptide. In some embodiments, the RNA guide is expressed from a plasmid that expresses the RNA guide but not a nuclease polypeptide. In some embodiments, the RNA guide is purchased from a commercial vendor. In some embodiments, the RNA guide is synthesized using one or more modified nucleotide, e.g., as described above.

C. Complexing

In some embodiments, an RNA guide is complexed with a nuclease polypeptide to form a ribonucleoprotein. In some embodiments, complexation of the RNA guide and nuclease polypeptide occurs at a temperature lower than about any one of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 50° C., or 55° C. In some embodiments, the RNA guide does not dissociate from the nuclease polypeptide at about 37° C. over an incubation period of at least about any one of 10 mins, 15 mins, 20 mins, 25 mins, 30 mins, 35 mins, 40 mins, 45 mins, 50 mins, 55 mins, 1 hr, 2 hr, 3 hr, 4 hr, or more hours.

In some embodiments, the RNA guide and nuclease polypeptide are complexed in a complexation buffer. In some embodiments, the nuclease polypeptide is stored in a buffer that is replaced with a complexation buffer to form a complex with the RNA guide. In some embodiments, the nuclease polypeptide is stored in a complexation buffer.

In some embodiments, the complexation buffer has a pH in a range of about 7.3 to 8.6. In one embodiment, the pH of the complexation buffer is about 7.3. In one embodiment, the pH of the complexation buffer is about 7.4. In one embodiment, the pH of the complexation buffer is about 7.5. In one embodiment, the pH of the complexation buffer is about 7.6. In one embodiment, the pH of the complexation buffer is about 7.7. In one embodiment, the pH of the complexation buffer is about 7.8. In one embodiment, the pH of the complexation buffer is about 7.9. In one embodiment, the pH of the complexation buffer is about 8.0. In one embodiment, the pH of the complexation buffer is about 8.1. In one embodiment, the pH of the complexation buffer is about 8.2. In one embodiment, the pH of the complexation buffer is about 8.3. In one embodiment, the pH of the complexation buffer is about 8.4. In one embodiment, the pH of the complexation buffer is about 8.5. In one embodiment, the pH of the complexation buffer is about 8.6.

In some embodiments, the nuclease polypeptide can be overexpressed and complexed with the RNA guide in a host cell prior to purification as described herein. In some embodiments, mRNA or DNA encoding the nuclease polypeptide is introduced into a cell so that the nuclease polypeptide is expressed in the cell. In some embodiments, the RNA guide is also introduced into the cell, whether simultaneously, separately, or sequentially from a single mRNA or DNA construct, such that the ribonucleoprotein complex is formed in the cell.

### III. Gene Editing Methods

The disclosure also provides methods of modifying a target site. In some embodiments, the methods comprise introducing a nuclease polypeptide and an RNA guide into a cell. The nuclease polypeptide and RNA guide can be introduced as a ribonucleoprotein complex into a cell. The nuclease polypeptide and RNA guide can be introduced on a nucleic acid vector. The nuclease polypeptide can be introduced as an mRNA. The RNA guide can be introduced directly into the cell. In some embodiments, the gene editing system described herein is delivered to a cell/tissue/person to reduce gene expression in the cell/tissue/person. In some embodiments, the gene editing system described herein is delivered to a cell/tissue/person to reduce protein levels in the cell/tissue/person.

A. Target Sequence

In some embodiments, the target nucleic acid is present in a cell. In some embodiments, the target nucleic acid is present in the nucleus of the cell. In some embodiments, the target nucleic acid is endogenous to the cell. In some embodiments, the target nucleic acid is a genomic DNA. In some embodiments, the target nucleic acid is a chromosomal DNA. In one embodiment, the target nucleic acid is an extrachromosomal nucleic acid. In some embodiments, the target nucleic acid is a protein-coding gene or a functional region thereof, such as a coding region, or a regulatory element, such as a promoter, enhancer, a 5′ or 3′ untranslated region, etc. In some embodiments, the target nucleic acid is a non-coding gene, such as transposon, miRNA, tRNA, ribosomal RNA, ribozyme, or lincRNA. In some embodiments, the target nucleic acid is a plasmid.

In some embodiments, the target nucleic acid is exogenous to a cell. In some embodiments, the target nucleic acid is a viral nucleic acid, such as viral DNA or viral RNA. In some embodiments, the target nucleic acid is a horizontally transferred plasmid. In some embodiments, the target nucleic acid is integrated in the genome of the cell. In some embodiments, the target nucleic acid is not integrated in the genome of the cell. In some embodiments, the target nucleic acid is a plasmid in the cell. In some embodiments, the target nucleic acid is present in an extrachromosomal array.

In some embodiments, the target nucleic acid is an isolated nucleic acid, such as an isolated DNA or an isolated RNA. In some embodiments, the target nucleic acid is present in a cell-free environment. In some embodiments, the target nucleic acid is an isolated vector, such as a plasmid. In some embodiments, the target nucleic acid is an ultrapure plasmid.

In some embodiments, the complex becomes activated upon binding to the target substrate. In some embodiments, the activated complex exhibits “multiple turnover” activity, whereby upon acting on (e.g., cleaving) the target nucleic acid, the activated complex remains in an activated state. In some embodiments, the activated complex exhibits “single turnover” activity, whereby upon acting on the target nucleic acid, the complex reverts to an inactive state.

In some embodiments, a nuclease described herein binds to a target nucleic acid at a sequence defined by the region of complementarity between the RNA guide and the target nucleic acid. In some embodiments, the PAM sequence of a nuclease described herein is located directly upstream of the target sequence of the target nucleic acid (e.g., directly 5′ of the target sequence). In some embodiments, the PAM sequence of a nuclease described herein is located directly 5′ of the non-complementary strand (e.g., non-target strand) of the target nucleic acid. As used herein, the “complementary strand” hybridizes to the RNA guide. As used herein, the “non-complementary strand” does not directly hybridize to the RNA.

In some embodiments, a nuclease of the present invention targets a target nucleic acid comprising a target sequence adjacent to a PAM sequence. In some embodiments, the PAM sequences corresponding to SEQ ID NOs: 1-32 are shown in Table 5.

TABLE 5
PAM Sequences.

| Nuclease polypeptide | PAM Sequence |
|---|---|
| SEQ ID NO: 1 | 5′-CC-3′ |
| | 5′-NCC-3′ |
| SEQ ID NO: 2 | 5′-TTC-3′ |
| | 5′-NTTC-3′ |
| SEQ ID NO: 3 | 5′-TY-3′ |
| | 5′-NTY-3′ |
| SEQ ID NO: 4 | 5′-ATC-3′ |
| | 5′-NATC-3′ |
| SEQ ID NO: 5 | 5′-CCN-3′ |
| | 5′-NCCN-3′ |
| SEQ ID NO: 6 | 5′-CCN-3′ |
| | 5′-NCCN-3′ |
| SEQ ID NO: 7 | 5′-CCN-3′ |
| | 5′-NCCN-3′ |
| SEQ ID NO: 8 | 5′-CCN-3′ |
| | 5′-NCCN-3′ |
| SEQ ID NO: 9 | 5′-CYN-3′ |
| | 5′-NCYN-3′ |
| SEQ ID NO: 10 | 5′-CCG-3′ |
| | 5′-NCCG-3′ |
| SEQ ID NO: 11 | 5′-CG-3′ |
| | 5′-NCG-3′ |
| SEQ ID NO: 12 | 5′-A-3′ |
| | 5′-NA-3′ |
| | 5′-NAA-3′ |
| SEQ ID NO: 13 | 5′-TA-3′ |
| | 5′-NTA-3′ |
| SEQ ID NO: 14 | 5′-TG-3′ |
| | 5′-NTG-3′ |
| SEQ ID NO: 15 | 5′-C-3′ |
| | 5′-NC-3′ |
| | 5′-NNC-3′ |
| SEQ ID NO: 16 | 5′-CC-3′ |
| | 5′-NCC-3′ |
| SEQ ID NO: 17 | 5′-CCNA-3′ |
| SEQ ID NO: 18 | 5′-CG-3′ |
| | 5′-NCG-3′ |
| SEQ ID NO: 19 | 5′-AAN-3′ |
| | 5′-NAAN-3′ |
| SEQ ID NO: 20 | 5′-AAG-3′ |
| | 5′-NAAG-3′ |
| SEQ ID NO: 21 | 5′-AAG-3′ |
| | 5′-NAAG-3′ |
| SEQ ID NO: 22 | 5′-TIN-3′ |
| | 5′-NTTN-3′ |
| SEQ ID NO: 23 | 5′-TIN-3′ |
| | 5′-NTTN-3′ |
| SEQ ID NO: 24 | 5′-GN-3′ |
| | 5′-NGN-3′ |
| SEQ ID NO: 25 | 5′-CT-3′ |
| | 5′-NCT-3′ |
| SEQ ID NO: 26 | 5′-TY-3′ |
| | 5′-NTY-3′ |
| SEQ ID NO: 27 | 5′-TY-3′ |
| | 5′-NTY-3′ |
| SEQ ID NO: 28 | 5′-TTC-3′ |
| | 5′-NTTC-3′ |
| SEQ ID NO: 29 | 5′-TY-3′ |
| | 5′-NTY-3′ |
| SEQ ID NO: 30 | 5′-GTN-3′ |
| | 5′-NGTN-3′ |
| SEQ ID NO: 31 | 5′-CCN-3′ |
| | 5′-NCCN-3′ |
| SEQ ID NO: 32 | 5′-CCY-3′ |
| | 5′-NCCY-3′ |

B. Delivery

Nucleases, RNA guides, tracrRNA sequences, sgRNA sequences, and/or gene editing systems described herein may be formulated, for example, including a carrier, such as a carrier and/or a polymeric carrier, e.g., a liposome, and delivered by known methods to a cell (e.g., a prokaryotic, eukaryotic, plant, mammalian, etc.). Such methods include, but not limited to, transfection (e.g., lipid-mediated, cationic polymers, calcium phosphate, dendrimers); electroporation or other methods of membrane disruption (e.g., nucleofection), viral delivery (e.g., lentivirus, retrovirus, adenovirus, AAV), microinjection, microprojectile bombardment (“gene gun”), fugene, direct sonic loading, cell squeezing, optical transfection, protoplast fusion, impalefection, magnetofection, exosome-mediated transfer, lipid nanoparticle-mediated transfer, and any combination thereof.

In some embodiments, the method comprises delivering one or more nucleic acids (e.g., nucleic acids encoding a nuclease, RNA guide, donor DNA, etc.), one or more transcripts thereof, and/or a pre-formed nuclease/RNA guide complex to a cell. Exemplary intracellular delivery methods, include, but are not limited to: viruses or virus-like agents; chemical-based transfection methods, such as those using calcium phosphate, dendrimers, liposomes, or cationic polymers (e.g., DEAE-dextran or polyethylenimine); non-chemical methods, such as microinjection, electroporation, cell squeezing, sonoporation, optical transfection, impalefection, protoplast fusion, bacterial conjugation, delivery of plasmids or transposons; particle-based methods, such as using a gene gun, magnectofection or magnet assisted transfection, particle bombardment; and hybrid methods, such as nucleofection. In some embodiments, the present application further provides cells produced by such methods, and organisms (such as animals, plants, or fungi) comprising or produced from such cells.

C. Genetically Modified Cells

The nucleases described herein can be introduced into a variety of cells. In some embodiments, the cell is an isolated cell. In some embodiments the cell is in cell culture. In some embodiments, the cell is ex vivo. In some embodiments, the cell is obtained from a living organism, and maintained in a cell culture. In some embodiments, the cell is a single-cellular organism.

In some embodiments, the cell is a prokaryotic cell. In some embodiments, the cell is a bacterial cell or derived from a bacterial cell. In some embodiments, the cell is an archaeal cell or derived from an archaeal cell.

In some embodiments, the cell is a eukaryotic cell. In some embodiments, the cell is a plant cell or derived from a plant cell. In some embodiments, the cell is a fungal cell or derived from a fungal cell. In some embodiments, the cell is an animal cell or derived from an animal cell. In some embodiments, the cell is an invertebrate cell or derived from an invertebrate cell. In some embodiments, the cell is a vertebrate cell or derived from a vertebrate cell. In some embodiments, the cell is a mammalian cell or derived from a mammalian cell. In some embodiments, the cell is a human cell. In some embodiments, the cell is a zebra fish cell. In some embodiments, the cell is a rodent cell. In some embodiments, the cell is synthetically made, sometimes termed an artificial cell.

In some embodiments, the cell is derived from a cell line. A wide variety of cell lines for tissue culture are known in the art. Examples of cell lines include, but are not limited to, 293T, MF7, K562, HeLa, and transgenic varieties thereof. Cell lines are available from a variety of sources known to those with skill in the art (see, e.g., the American Type Culture Collection (ATCC) (Manassas, Va.)). In some embodiments, a cell transfected with one or more nucleic acids (such as nuclease polypeptide encoding vector and RNA guide) is used to establish a new cell line comprising one or more vector-derived sequences to establish a new cell line comprising modification to the target nucleic acid or target locus. In some embodiments, the cell is an immortal or immortalized cell.

In some embodiments, the method comprises introducing into a host cell one or more nucleic acids comprising nucleotide sequences encoding a DNA-targeting RNA (e.g., RNA guide) and/or the nuclease. In one embodiment, a cell comprising a target DNA is in vitro, in vivo, or ex vivo. In other embodiments, nucleic acids comprising nucleotide sequences encoding a DNA-targeting RNA (e.g., RNA guide) and/or the nuclease include recombinant expression vectors e.g., including but not limited to adeno-associated virus constructs, recombinant adenoviral constructs, recombinant lentiviral constructs, recombinant retroviral constructs, and the like.

In some embodiments, the cell is a primary cell. In some embodiments, the cell is a stem cell such as a totipotent stem cell (e.g., omnipotent), a pluripotent stem cell, a multipotent stem cell, an oligopotent stem cell, or an unipotent stem cell. In some embodiments, the cell is an induced pluripotent stem cell (iPSC) or derived from an iPSC. In some embodiments, the cell is a differentiated cell. For example, in some embodiments, the differentiated cell is a muscle cell (e.g., a myocyte), a fat cell (e.g., an adipocyte), a bone cell (e.g., an osteoblast, osteocyte, osteoclast), a blood cell (e.g., a monocyte, a lymphocyte, a neutrophil, an eosinophil, a basophil, a macrophage, a erythrocyte, or a platelet), a nerve cell (e.g., a neuron), an epithelial cell, an immune cell (e.g., a lymphocyte, a neutrophil, a monocyte, or a macrophage), a liver cell (e.g., a hepatocyte), a fibroblast, or a sex cell. In some embodiments, the cell is a terminally differentiated cell. For example, in some embodiments, the terminally differentiated cell is a neuronal cell, an adipocyte, a cardiomyocyte, a skeletal muscle cell, an epidermal cell, or a gut cell. In some embodiments, the cell is a mammalian cell, e.g., a human cell or a murine cell. In some embodiments, the murine cell is derived from a wild-type mouse, an immunosuppressed mouse, or a disease-specific mouse model.

### IV. Kits and Uses Thereof

The invention also provides kits that can be used, for example, to carry out a method described herein. In some embodiments, the kits include a nuclease of the present invention. In some embodiments, the kits include a polynucleotide that encodes such a nuclease, and optionally the polynucleotide is comprised within a vector, e.g., as described herein. The kits also can optionally include an RNA guide, e.g., as described herein. The RNA guide of the kits of the invention can be designed to target a sequence of interest, as is known in the art. The nuclease and the RNA guide can be packaged within the same vial or other vessel within a kit or can be packaged in separate vials or other vessels, the contents of which can be mixed prior to use. The kits can additionally include, optionally, a buffer and/or instructions for use of the nuclease and/or RNA guide.

Gene editing systems, vectors, nucleic acids, RNA guides and cells disclosed herein may be used in therapy. Gene editing systems, vectors, nucleic acids, RNA guides and cells disclosed herein may be used in methods of treating a disease or condition in a subject. Any suitable delivery or administration method known in the art may be used to deliver the gene editing systems, vectors, nucleic acids, RNA guides and cells disclosed herein. Such methods may involve contacting a target sequence with a gene editing system, vector, nucleic acid, or RNA guide disclosed herein. In some embodiments, a cell engineered using an RNA guide disclosed herein is used for ex vivo gene therapy.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds.(1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; and B. Perbal, A practical Guide To Molecular Cloning (1984); F. M. Ausubel et al. (eds.).

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

The following examples are provided to further illustrate some embodiments of the present invention but are not intended to limit the scope of the invention; it will be understood by their exemplary nature that other procedures, methodologies, or techniques known to those skilled in the art may alternatively be used.

### Example 1—Expression of Nuclease Polypeptides in E. coli

In this Example, a system individually comprising a nuclease of any one of SEQ ID NOs: 1-32 is engineered and introduced into E. coli.

For each nuclease, a polynucleotide encoding the nuclease is E. coli codon-optimized, synthesized (Genscript), and individually cloned into a custom expression system derived from pET-28a(+) (EMD-Millipore). The vector includes a polynucleotide encoding each nuclease under the control of a lac promoter and an E. coli ribosome binding sequence. The vector also includes sites for a tracrRNA (Table 3) and an RNA guide (with a direct repeat of Table 2) or a sgRNA (Table 4) following the open reading frame for the nuclease. Plasmid configurations are shown in Table 6. The spacers are designed to target sequences of a pACYC184 plasmid and E. coli essential genes.

TABLE 6
Bacterial Plasmids.

| Configuration | Activity |
|---|---|
| Nuclease-TracrRNA-full length DR-spacer- | Expect activity |
| full length DR | |
| Nuclease-TracrRNA-mature DR-spacer | |
| Nuclease-TracrRNA-full length DR-spacer- | Do not expect activity |
| full length DR | |
| Nuclease-TracrRNA-mature DR-spacer | |
| Nuclease-TracrRNA-sgRNA | Expect activity |
| Nuclease-sgRNA | Expect activity |

The plasmids described in Table 6 are electroporated into E. Cloni electrocompetent E. coli (Lucigen). The plasmids are either co-transformed with purified pACYC184 plasmid or directly transformed into pACYC184-containing E. Cloni electrocompetent E. coli (Lucigen), plated onto agar containing the proper antibiotics, and incubated for 10-12 hours at 37° C.

A proxy for activity of the engineered nuclease systems in E. coli is investigated, wherein bacterial cell death is used as the proxy for system activity. An active nuclease associated with an RNA guide and tracrRNA or with an sgRNA can disrupt expression of a spacer sequence target, e.g., a pACYC184 plasmid sequence or an E. coli essential gene, resulting in cell death. Using this proxy, the activity of the nucleases disclosed herein can be determined in E. coli.

### Example 2—Expression of Nuclease Polypeptides in Mammalian Cells

This Example describes an indel assessment on mammalian targets by the nuclease of SEQ ID NOs: 1-32 introduced into mammalian cells by transient transfection.

The nucleases of SEQ ID NOs: 1-32 are individually cloned into a pcda3.1 backbone (Invitrogen™). The plasmids are then maxi-prepped and diluted. The sgRNA sequences set forth in Table 4 are further individually cloned into a pUC19 backbone (New England Biolabs®) under a U6 promoter, purified, and diluted. Targets are selected to be adjacent to the PAM sequences set forth in Table 5.

Approximately 16 hours prior to transfection, 25,000 HEK293T cells in DMEM/10% FBS+Pen/Strep are plated into each well of a 96-well plate. On the day of transfection, the cells are 70-90% confluent. For each well to be transfected, a mixture of Lipofectamine™ 2000 (ThermoFisher®) and Opti-MEM™ (ThermoFisher®) is prepared and then incubated at room temperature for 5-20 minutes (Solution 1). After incubation, the Lipofectamine™:OptiMEM™ mixture is added to a separate mixture containing nuclease plasmid, sgRNA, and water (Solution 2). In the case of negative controls, the sgRNA is not included in Solution 2. The solution 1 and solution 2 mixtures are mixed by pipetting up and down and then incubated at room temperature. Following incubation, Solution 1 and Solution 2 mixture are added dropwise to each well of a 96 well plate containing the cells. 72 hours post transfection, cells are trypsinized by adding TrypLE™ (ThermoFisher®) to the center of each well and incubated for approximately 5 minutes. D10 media is then added to each well and mixed to resuspend cells. The cells are then spun down for 10 minutes, and the supernatant is discarded. QuickExtract™ extraction reagent (Biosearch™ Technologies) is added to ⅕ the amount of the original cell suspension volume. The resuspended cell solution is incubated at 65° C. for 15 minutes, 68° C. for 15 minutes, and 98° C. for 10 minutes.

Samples for Next Generation Sequencing are prepared by two rounds of PCR. The first round (PCR1) is used to amplify specific genomic regions depending on the target. PCR1 products are purified by column purification. Round 2 PCR (PCR2) is done to add Illumina adapters and indexes. Reactions are then pooled and purified by column purification. Sequencing runs are done with a 150 cycle NextSeq v2.5 mid or high output kit.

Presence of indels at the analyzed targets, as determined by NGS, is indicative of mammalian activity of the nucleases of SEQ ID NOs: 1-32 with the sgRNA sequences of Table 4.

### Example 3—Activity of Nucleases of SEQ ID NO: 26 and SEQ ID NO: 27 in Mammalian Cells

This Example describes an indel assessment on mammalian targets by the nucleases of SEQ ID NO: 26 and SEQ ID NO: 27 introduced into mammalian cells by transient transfection.

The nucleic acid of SEQ ID NO: 58, which encodes the nuclease of SEQ ID NO: 26, and the nucleic acid of SEQ ID NO: 59, which encodes the nuclease of SEQ ID NO: 27 were individually cloned into pcda3.1 backbones (Invitrogen™). The plasmids were then maxi-prepped and diluted. The sgRNA sequences set forth in Table 7 were further individually cloned into a pUC19 backbone (New England Biolabs®) under a U6 promoter, purified, and diluted. The target and PAM sequences are also shown in Table 7.

TABLE 7
sgRNA and Target Sequences

| Target | PAM | sgRNA for nuclease | sgRNA for nuclease |
|---|---|---|---|
| sequence | sequence | of SEQ ID NO: 26 | of SEQ ID NO: 27 |
| AAVS1_T1 | 5′-CTTC-3′ | CUUAUCGUGAGGUGCAGC | CUUAUCGUGAGGUGCAGC |
| CAGCTGACT | | CACGAUGUGCGAAUCUAA | CACGAUGUGCGAAUCUAA |
| TGGATGCTG | | AGUGGACAGAAAUGGGAA | AGUGAACAGAAAUGGGAA |
| GA (SEQ ID | | CAGACGUUCGCACGAAUG | CAGACGUUCGCACGAAUG |
| NO: 470) | | UUGGCGGAUUUCUUCGGA | UUGGCGGAUUUCUUCGGA |
| | | AAUCGAGCCAAUCAUCAU | AAUCGAGCCAAUAAUCAU |
| | | AUCCAUAUUAUCUGCGGA | AUCCAUAUUACCGGGUUG |
| | | UGGAUGUGAACUGCAAGC | CUGCGGAUGGAUGUGAAC |
| | | AGCUGACUUGGAUGCUGG | UGCAAGCAGCUGACUUGG |
| | | A (SEQ ID NO: 479) | AUGCUGGA (SEQ ID NO: |
| | | | 480) |
| AAVS1_T2 | 5′-GTTC-3′ | CUUAUCGUGAGGUGCAGC | CUUAUCGUGAGGUGCAGC |
| CTTCTCTCT | | CACGAUGUGCGAAUCUAA | CACGAUGUGCGAAUCUAA |
| GTCCCTCCC | | AGUGGACAGAAAUGGGAA | AGUGAACAGAAAUGGGAA |
| TT (SEQ ID | | CAGACGUUCGCACGAAUG | CAGACGUUCGCACGAAUG |
| NO: 471) | | UUGGCGGAUUUCUUCGGA | UUGGCGGAUUUCUUCGGA |
| | | AAUCGAGCCAAUCAUCAU | AAUCGAGCCAAUAAUCAU |
| | | AUCCAUAUUAUCUGCGGA | AUCCAUAUUACCGGGUUG |
| | | UGGAUGUGAACUGCAAGC | CUGCGGAUGGAUGUGAAC |
| | | UUCUCUCUGUCCCUCCCUU | UGCAAGCUUCUCUCUGUC |
| | | (SEQ ID NO: 481) | CCUCCCUU (SEQ ID NO: |
| | | | 482) |
| AAVS1_T3 | 5′-TTTC-3′ | CUUAUCGUGAGGUGCAGC | CUUAUCGUGAGGUGCAGC |
| CACCCAGTT | | CACGAUGUGCGAAUCUAA | CACGAUGUGCGAAUCUAA |
| GTCATGGCG | | AGUGGACAGAAAUGGGAA | AGUGAACAGAAAUGGGAA |
| AT (SEQ ID | | CAGACGUUCGCACGAAUG | CAGACGUUCGCACGAAUG |
| NO: 472) | | UUGGCGGAUUUCUUCGGA | UUGGCGGAUUUCUUCGGA |
| | | AAUCGAGCCAAUCAUCAU | AAUCGAGCCAAUAAUCAU |
| | | AUCCAUAUUAUCUGCGGA | AUCCAUAUUACCGGGUUG |
| | | UGGAUGUGAACUGCAAGC | CUGCGGAUGGAUGUGAAC |
| | | ACCCAGUUGUCAUGGCGA | UGCAAGCACCCAGUUGUC |
| | | U (SEQ ID NO: 483) | AUGGCGAU (SEQ ID NO: |
| | | | 484) |
| EMX1_T1 | 5′-CTTC-3′ | CUUAUCGUGAGGUGCAGC | CUUAUCGUGAGGUGCAGC |
| CCAGGTGGG | | CACGAUGUGCGAAUCUAA | CACGAUGUGCGAAUCUAA |
| CAAACACGA | | AGUGGACAGAAAUGGGAA | AGUGAACAGAAAUGGGAA |
| TT (SEQ ID | | CAGACGUUCGCACGAAUG | CAGACGUUCGCACGAAUG |
| NO: 473) | | UUGGCGGAUUUCUUCGGA | UUGGCGGAUUUCUUCGGA |
| | | AAUCGAGCCAAUCAUCAU | AAUCGAGCCAAUAAUCAU |
| | | AUCCAUAUUAUCUGCGGA | AUCCAUAUUACCGGGUUG |
| | | UGGAUGUGAACUGCAAGC | CUGCGGAUGGAUGUGAAC |
| | | CAGGUGGGCAAACACGAU | UGCAAGCCAGGUGGGCAA |
| | | U (SEQ ID NO: 485) | ACACGAUU (SEQ ID NO: |
| | | | 486) |
| EMX1_T2 | 5′-GTTC-3′ | CUUAUCGUGAGGUGCAGC | CUUAUCGUGAGGUGCAGC |
| TTGAGGGAA | | CACGAUGUGCGAAUCUAA | CACGAUGUGCGAAUCUAA |
| AAATCCAAC | | AGUGGACAGAAAUGGGAA | AGUGAACAGAAAUGGGAA |
| TG (SEQ ID | | CAGACGUUCGCACGAAUG | CAGACGUUCGCACGAAUG |
| NO: 474) | | UUGGCGGAUUUCUUCGGA | UUGGCGGAUUUCUUCGGA |
| | | AAUCGAGCCAAUCAUCAU | AAUCGAGCCAAUAAUCAU |
| | | AUCCAUAUUAUCUGCGGA | AUCCAUAUUACCGGGUUG |
| | | UGGAUGUGAACUGCAAGU | CUGCGGAUGGAUGUGAAC |
| | | UGAGGGAAAAAUCCAACU | UGCAAGUUGAGGGAAAAA |
| | | G (SEQ ID NO: 487) | UCCAACUG (SEQ ID NO: |
| | | | 488) |
| EMX1_T3 | 5′-TTTC-3′ | CUUAUCGUGAGGUGCAGC | CUUAUCGUGAGGUGCAGC |
| TGTTGCCCT | | CACGAUGUGCGAAUCUAA | CACGAUGUGCGAAUCUAA |
| CATAACTTA | | AGUGGACAGAAAUGGGAA | AGUGAACAGAAAUGGGAA |
| TC (SEQ ID | | CAGACGUUCGCACGAAUG | CAGACGUUCGCACGAAUG |
| NO: 475) | | UUGGCGGAUUUCUUCGGA | UUGGCGGAUUUCUUCGGA |
| | | AAUCGAGCCAAUCAUCAU | AAUCGAGCCAAUAAUCAU |
| | | AUCCAUAUUAUCUGCGGA | AUCCAUAUUACCGGGUUG |
| | | UGGAUGUGAACUGCAAGU | CUGCGGAUGGAUGUGAAC |
| | | GUUGCCCUCAUAACUUAU | UGCAAGUGUUGCCCUCAU |
| | | C (SEQ ID NO: 489) | AACUUAUC (SEQ ID NO: |
| | | | 490) |
| VEGFA_T1 | 5′-CTTC-3′ | CUUAUCGUGAGGUGCAGC | CUUAUCGUGAGGUGCAGC |
| TCCTGCTGA | | CACGAUGUGCGAAUCUAA | CACGAUGUGCGAAUCUAA |
| CATGACAAA | | AGUGGACAGAAAUGGGAA | AGUGAACAGAAAUGGGAA |
| TA (SEQ ID | | CAGACGUUCGCACGAAUG | CAGACGUUCGCACGAAUG |
| NO: 476) | | UUGGCGGAUUUCUUCGGA | UUGGCGGAUUUCUUCGGA |
| | | AAUCGAGCCAAUCAUCAU | AAUCGAGCCAAUAAUCAU |
| | | AUCCAUAUUAUCUGCGGA | AUCCAUAUUACCGGGUUG |
| | | UGGAUGUGAACUGCAAGU | CUGCGGAUGGAUGUGAAC |
| | | CCUGCUGACAUGACAAAU | UGCAAGUCCUGCUGACAU |
| | | A (SEQ ID NO: 491) | GACAAAUA (SEQ ID NO: |
| | | | 492) |
| VEGFA_T2 | 5′-GTTC-3′ | CUUAUCGUGAGGUGCAGC | CUUAUCGUGAGGUGCAGC |
| TGTCGCAGT | | CACGAUGUGCGAAUCUAA | CACGAUGUGCGAAUCUAA |
| TGCAAATGA | | AGUGGACAGAAAUGGGAA | AGUGAACAGAAAUGGGAA |
| AG (SEQ ID | | CAGACGUUCGCACGAAUG | CAGACGUUCGCACGAAUG |
| NO: 477) | | UUGGCGGAUUUCUUCGGA | UUGGCGGAUUUCUUCGGA |
| | | AAUCGAGCCAAUCAUCAU | AAUCGAGCCAAUAAUCAU |
| | | AUCCAUAUUAUCUGCGGA | AUCCAUAUUACCGGGUUG |
| | | UGGAUGUGAACUGCAAGU | CUGCGGAUGGAUGUGAAC |
| | | GUCGCAGUUGCAAAUGAA | UGCAAGUGUCGCAGUUGC |
| | | G (SEQ ID NO: 493) | AAAUGAAG (SEQ ID NO: |
| | | | 494) |
| VEGFA_T3 | 5′-TTTC-3′ | CUUAUCGUGAGGUGCAGC | CUUAUCGUGAGGUGCAGC |
| TAGAAGCTG | | CACGAUGUGCGAAUCUAA | CACGAUGUGCGAAUCUAA |
| ATCTGAAGG | | AGUGGACAGAAAUGGGAA | AGUGAACAGAAAUGGGAA |
| GA (SEQ ID | | CAGACGUUCGCACGAAUG | CAGACGUUCGCACGAAUG |
| NO: 478) | | UUGGCGGAUUUCUUCGGA | UUGGCGGAUUUCUUCGGA |
| | | AAUCGAGCCAAUCAUCAU | AAUCGAGCCAAUAAUCAU |
| | | AUCCAUAUUAUCUGCGGA | AUCCAUAUUACCGGGUUG |
| | | UGGAUGUGAACUGCAAGU | CUGCGGAUGGAUGUGAAC |
| | | AGAAGCUGAUCUGAAGGG | UGCAAGUAGAAGCUGAUC |
| | | A (SEQ ID NO: 495) | UGAAGGGA (SEQ ID NO: |
| | | | 496) |

Approximately 16 hours prior to transfection, 25,000 HEK293T cells in DMEM/10% FBS+Pen/Strep were plated into each well of a 96-well plate. On the day of transfection, the cells were 70-90% confluent. For each well to be transfected, a mixture of Lipofectamine™ 2000 (ThermoFisher®) and Opti-MEM™ (ThermoFisher®) was prepared and then incubated at room temperature for 5-20 minutes (Solution 1). After incubation, the Lipofectamine™:OptiMEM™ mixture was added to a separate mixture containing nuclease plasmid, sgRNA, and water (Solution 2). In the case of negative controls, the sgRNA was not included in Solution 2. The solution 1 and solution 2 mixtures were mixed by pipetting up and down and then incubated at room temperature for 25 minutes. Following incubation, Solution 1 and Solution 2 mixture were added dropwise to each well of a 96 well plate containing the cells. 72 hours post transfection, cells were trypsinized by adding TrypLE™ (ThermoFisher®) to the center of each well and incubated for approximately 5 minutes. D10 media was then added to each well and mixed to resuspend cells. The cells were then spun down for 10 minutes, and the supernatant was discarded. QuickExtract™ extraction reagent (Biosearch™ Technologies) was added to ⅕ the amount of the original cell suspension volume. The resuspended cell solution was incubated at 65° C. for 15 minutes, 68° C. for 15 minutes, and 98° C. for 10 minutes.

NGS samples were prepared by two rounds of PCR. The first round (PCR1) was used to amplify specific genomic regions depending on the target. PCR1 products were purified by column purification. Round 2 PCR (PCR2) was done to add Illumina adapters and indexes. Reactions were then pooled and purified by column purification. Sequencing runs were done with a 150 cycle NextSeq v2.5 mid or high output kit.

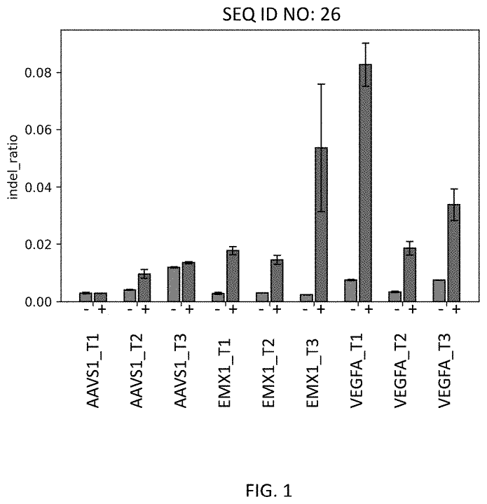

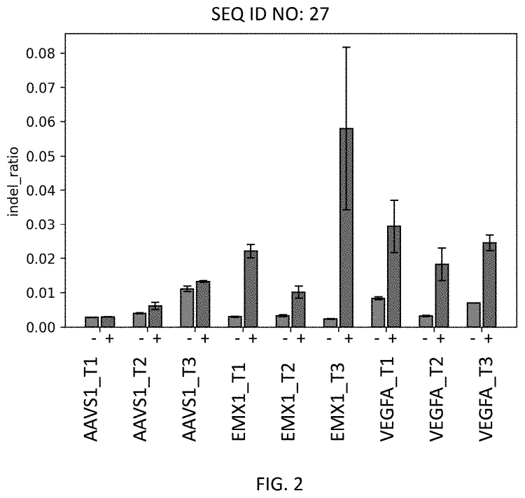

FIG. 1 and FIG. 2 show indel activity of SEQ ID NO: 26 and SEQ ID NO: 27, respectively, across nine mammalian targets. Indel ratio, calculated as the fraction of NGS reads comprising an indel, is shown on the y-axis. For the nucleases of SEQ ID NO: 26 and SEQ ID NO: 27, experimental (“+”) samples exhibited higher indel ratios than those of the negative control (“−”) samples at most of the analyzed target sites. As shown in FIG. 1, the nuclease of SEQ ID NO: 26 induced indels in up to about 8% of NGS reads, and as shown in FIG. 2, the nuclease of SEQ ID NO: 27 induced indels in up to about 6% of NGS reads. Additionally, the nucleases of SEQ ID NO: 26 and SEQ ID NO: 27 were capable of recognizing the PAM sequences of 5′-CTTC-3′, 5 ‘-GTTC-3’, and 5′-TTTC-3′.

Therefore, this Example shows that the polypeptides of SEQ ID NO: 26 and SEQ ID NO: 27 are active nucleases in mammalian cells.

### Enumerated Embodiments

The following enumerated embodiments are provided, the numbering of which is not to be construed as designating levels of importance.

Embodiment 1 provides a gene editing system comprising:
(a) a nuclease or a first nucleic acid encoding the nuclease, wherein the nuclease comprises an amino acid sequence with at least 80% identity to any one of SEQ ID NOs: 1-32; and
(b) an RNA guide or a second nucleic acid encoding the RNA guide, wherein the RNA guide comprises a direct repeat sequence and a spacer sequence,
wherein the nuclease binds to the RNA guide, and wherein the spacer sequence is specific to a target sequence within a target nucleic acid.

Embodiment 2 provides the gene editing system of embodiment 1, wherein the nuclease comprises a RuvC domain or a split RuvC domain.

Embodiment 3 provides the gene editing system of embodiment 1 or 2, wherein the nuclease comprises a catalytic residue (e.g., aspartic acid or glutamic acid).

Embodiment 4 provides the gene editing system of any one of embodiments 1-3, wherein the nuclease comprises an amino acid sequence with at least 95% identity to any one of SEQ ID NOs: 1-32.

Embodiment 5 provides the gene editing system of any one of embodiments 1-4, wherein:
(a) the nuclease comprises an amino acid sequence with at least 95% identity to SEQ ID NO: 1 and the direct repeat sequence comprises a nucleotide sequence with at least 90% identity to SEQ ID NO: 65 or SEQ ID NO: 66,
(b) the nuclease comprises an amino acid sequence with at least 95% identity to SEQ ID NO: 2 and the direct repeat sequence comprises a nucleotide sequence with at least 90% identity to SEQ ID NO: 67 or SEQ ID NO: 68,
(c) the nuclease comprises an amino acid sequence with at least 95% identity to SEQ ID NO: 3 and the direct repeat sequence comprises a nucleotide sequence with at least 90% identity to SEQ ID NO: 69 or SEQ ID NO: 70,
(d) the nuclease comprises an amino acid sequence with at least 95% identity to SEQ ID NO: 4 and the direct repeat sequence comprises a nucleotide sequence with at least 90% identity to SEQ ID NO: 71 or SEQ ID NO: 72,
(e) the nuclease comprises an amino acid sequence with at least 95% identity to SEQ ID NO: 5 and the direct repeat sequence comprises a nucleotide sequence with at least 90% identity to SEQ ID NO: 73 or SEQ ID NO: 74,
(f) the nuclease comprises an amino acid sequence with at least 95% identity to SEQ ID NO: 6 and the direct repeat sequence comprises a nucleotide sequence with at least 90% identity to SEQ ID NO: 75 or SEQ ID NO: 76,
(g) the nuclease comprises an amino acid sequence with at least 95% identity to SEQ ID NO: 7 and the direct repeat sequence comprises a nucleotide sequence with at least 90% identity to SEQ ID NO: 77 or SEQ ID NO: 78,
(h) the nuclease comprises an amino acid sequence with at least 95% identity to SEQ ID NO: 8 and the direct repeat sequence comprises a nucleotide sequence with at least 90% identity to SEQ ID NO: 79 or SEQ ID NO: 80,
(i) the nuclease comprises an amino acid sequence with at least 95% identity to SEQ ID NO: 9 and the direct repeat sequence comprises a nucleotide sequence with at least 90% identity to SEQ ID NO: 81 or SEQ ID NO: 82,
(j) the nuclease comprises an amino acid sequence with at least 95% identity to SEQ ID NO: 10 and the direct repeat sequence comprises a nucleotide sequence with at least 90% identity to SEQ ID NO: 83 or SEQ ID NO: 84,
(k) the nuclease comprises an amino acid sequence with at least 95% identity to SEQ ID NO: 11 and the direct repeat sequence comprises a nucleotide sequence with at least 90% identity to SEQ ID NO: 85 or SEQ ID NO: 86,
(l) the nuclease comprises an amino acid sequence with at least 95% identity to SEQ ID NO: 12 and the direct repeat sequence comprises a nucleotide sequence with at least 90% identity to SEQ ID NO: 87 or SEQ ID NO: 88,
(m) the nuclease comprises an amino acid sequence with at least 95% identity to SEQ ID NO: 13 and the direct repeat sequence comprises a nucleotide sequence with at least 90% identity to SEQ ID NO: 89 or SEQ ID NO: 90,
(n) the nuclease comprises an amino acid sequence with at least 95% identity to SEQ ID NO: 14 and the direct repeat sequence comprises a nucleotide sequence with at least 90% identity to SEQ ID NO: 91 or SEQ ID NO: 92,
(o) the nuclease comprises an amino acid sequence with at least 95% identity to SEQ ID NO: 15 and the direct repeat sequence comprises a nucleotide sequence with at least 90% identity to SEQ ID NO: 93 or SEQ ID NO: 94,
(p) the nuclease comprises an amino acid sequence with at least 95% identity to SEQ ID NO: 16 and the direct repeat sequence comprises a nucleotide sequence with at least 90% identity to SEQ ID NO: 95 or SEQ ID NO: 96,
(q) the nuclease comprises an amino acid sequence with at least 95% identity to SEQ ID NO: 17 and the direct repeat sequence comprises a nucleotide sequence with at least 90% identity to SEQ ID NO: 97 or SEQ ID NO: 98,
(r) the nuclease comprises an amino acid sequence with at least 95% identity to SEQ ID NO: 18 and the direct repeat sequence comprises a nucleotide sequence with at least 90% identity to SEQ ID NO: 99 or SEQ ID NO: 100,
(s) the nuclease comprises an amino acid sequence with at least 95% identity to SEQ ID NO: 19 and the direct repeat sequence comprises a nucleotide sequence with at least 90% identity to SEQ ID NO: 101 or SEQ ID NO: 102,
(t) the nuclease comprises an amino acid sequence with at least 95% identity to SEQ ID NO: 20 and the direct repeat sequence comprises a nucleotide sequence with at least 90% identity to SEQ ID NO: 103 or SEQ ID NO: 104,
(u) the nuclease comprises an amino acid sequence with at least 95% identity to SEQ ID NO: 21 and the direct repeat sequence comprises a nucleotide sequence with at least 90% identity to SEQ ID NO: 105 or SEQ ID NO: 106,
(v) the nuclease comprises an amino acid sequence with at least 95% identity to SEQ ID NO: 22 and the direct repeat sequence comprises a nucleotide sequence with at least 90% identity to SEQ ID NO: 107 or SEQ ID NO: 108,
(w) the nuclease comprises an amino acid sequence with at least 95% identity to SEQ ID NO: 23 and the direct repeat sequence comprises a nucleotide sequence with at least 90% identity to SEQ ID NO: 109 or SEQ ID NO: 110,
(x) the nuclease comprises an amino acid sequence with at least 95% identity to SEQ ID NO: 24 and the direct repeat sequence comprises a nucleotide sequence with at least 90% identity to SEQ ID NO: 111 or SEQ ID NO: 112,
(y) the nuclease comprises an amino acid sequence with at least 95% identity to SEQ ID NO: 25 and the direct repeat sequence comprises a nucleotide sequence with at least 90% identity to SEQ ID NO: 113 or SEQ ID NO: 114,
(z) the nuclease comprises an amino acid sequence with at least 95% identity to SEQ ID NO: 26 and the direct repeat sequence comprises a nucleotide sequence with at least 90% identity to SEQ ID NO: 115 or SEQ ID NO: 116,
(aa) the nuclease comprises an amino acid sequence with at least 95% identity to SEQ ID NO: 27 and the direct repeat sequence comprises a nucleotide sequence with at least 90% identity to SEQ ID NO: 117 or SEQ ID NO: 118,
(bb) the nuclease comprises an amino acid sequence with at least 95% identity to SEQ ID NO: 28 and the direct repeat sequence comprises a nucleotide sequence with at least 90% identity to SEQ ID NO: 119 or SEQ ID NO: 120,
(cc) the nuclease comprises an amino acid sequence with at least 95% identity to SEQ ID NO: 29 and the direct repeat sequence comprises a nucleotide sequence with at least 90% identity to SEQ ID NO: 121 or SEQ ID NO: 122,
(dd) the nuclease comprises an amino acid sequence with at least 95% identity to SEQ ID NO: 30 and the direct repeat sequence comprises a nucleotide sequence with at least 90% identity to SEQ ID NO: 123 or SEQ ID NO: 124,
(ee) the nuclease comprises an amino acid sequence with at least 95% identity to SEQ ID NO: 31 and the direct repeat sequence comprises a nucleotide sequence with at least 90% identity to SEQ ID NO: 125 or SEQ ID NO: 126; or
(ff) the nuclease comprises an amino acid sequence with at least 95% identity to SEQ ID NO: 32 and the direct repeat sequence comprises a nucleotide sequence with at least 90% identity to SEQ ID NO: 127 or SEQ ID NO: 128.

Embodiment 6. The gene editing system of any one of embodiments 1-5, wherein:
(a) the nuclease comprises an amino acid sequence with at least 95% identity to SEQ ID NO: 1 and the direct repeat sequence comprises a nucleotide sequence with at least 95% identity to SEQ ID NO: 65 or SEQ ID NO: 66,
(b) the nuclease comprises an amino acid sequence with at least 95% identity to SEQ ID NO: 2 and the direct repeat sequence comprises a nucleotide sequence with at least 95% identity to SEQ ID NO: 67 or SEQ ID NO: 68,
(c) the nuclease comprises an amino acid sequence with at least 95% identity to SEQ ID NO: 3 and the direct repeat sequence comprises a nucleotide sequence with at least 95% identity to SEQ ID NO: 69 or SEQ ID NO: 70,
(d) the nuclease comprises an amino acid sequence with at least 95% identity to SEQ ID NO: 4 and the direct repeat sequence comprises a nucleotide sequence with at least 95% identity to SEQ ID NO: 71 or SEQ ID NO: 72,
(e) the nuclease comprises an amino acid sequence with at least 95% identity to SEQ ID NO: 5 and the direct repeat sequence comprises a nucleotide sequence with at least 95% identity to SEQ ID NO: 73 or SEQ ID NO: 74,
(f) the nuclease comprises an amino acid sequence with at least 95% identity to SEQ ID NO: 6 and the direct repeat sequence comprises a nucleotide sequence with at least 95% identity to SEQ ID NO: 75 or SEQ ID NO: 76,
(g) the nuclease comprises an amino acid sequence with at least 95% identity to SEQ ID NO: 7 and the direct repeat sequence comprises a nucleotide sequence with at least 95% identity to SEQ ID NO: 77 or SEQ ID NO: 78,
(h) the nuclease comprises an amino acid sequence with at least 95% identity to SEQ ID NO: 8 and the direct repeat sequence comprises a nucleotide sequence with at least 95% identity to SEQ ID NO: 79 or SEQ ID NO: 80,
(i) the nuclease comprises an amino acid sequence with at least 95% identity to SEQ ID NO: 9 and the direct repeat sequence comprises a nucleotide sequence with at least 95% identity to SEQ ID NO: 81 or SEQ ID NO: 82,
(j) the nuclease comprises an amino acid sequence with at least 95% identity to SEQ ID NO: 10 and the direct repeat sequence comprises a nucleotide sequence with at least 95% identity to SEQ ID NO: 83 or SEQ ID NO: 84,
(k) the nuclease comprises an amino acid sequence with at least 95% identity to SEQ ID NO: 11 and the direct repeat sequence comprises a nucleotide sequence with at least 95% identity to SEQ ID NO: 85 or SEQ ID NO: 86,
(l) the nuclease comprises an amino acid sequence with at least 95% identity to SEQ ID NO: 12 and the direct repeat sequence comprises a nucleotide sequence with at least 95% identity to SEQ ID NO: 87 or SEQ ID NO: 88,
(m) the nuclease comprises an amino acid sequence with at least 95% identity to SEQ ID NO: 13 and the direct repeat sequence comprises a nucleotide sequence with at least 95% identity to SEQ ID NO: 89 or SEQ ID NO: 90,
(n) the nuclease comprises an amino acid sequence with at least 95% identity to SEQ ID NO: 14 and the direct repeat sequence comprises a nucleotide sequence with at least 95% identity to SEQ ID NO: 91 or SEQ ID NO: 92,
(o) the nuclease comprises an amino acid sequence with at least 95% identity to SEQ ID NO: 15 and the direct repeat sequence comprises a nucleotide sequence with at least 95% identity to SEQ ID NO: 93 or SEQ ID NO: 94,
(p) the nuclease comprises an amino acid sequence with at least 95% identity to SEQ ID NO: 16 and the direct repeat sequence comprises a nucleotide sequence with at least 95% identity to SEQ ID NO: 95 or SEQ ID NO: 96,
(q) the nuclease comprises an amino acid sequence with at least 95% identity to SEQ ID NO: 17 and the direct repeat sequence comprises a nucleotide sequence with at least 95% identity to SEQ ID NO: 97 or SEQ ID NO: 98,
(r) the nuclease comprises an amino acid sequence with at least 95% identity to SEQ ID NO: 18 and the direct repeat sequence comprises a nucleotide sequence with at least 95% identity to SEQ ID NO: 99 or SEQ ID NO: 100,
(s) the nuclease comprises an amino acid sequence with at least 95% identity to SEQ ID NO: 19 and the direct repeat sequence comprises a nucleotide sequence with at least 95% identity to SEQ ID NO: 101 or SEQ ID NO: 102,
(t) the nuclease comprises an amino acid sequence with at least 95% identity to SEQ ID NO: 20 and the direct repeat sequence comprises a nucleotide sequence with at least 95% identity to SEQ ID NO: 103 or SEQ ID NO: 104,
(u) the nuclease comprises an amino acid sequence with at least 95% identity to SEQ ID NO: 21 and the direct repeat sequence comprises a nucleotide sequence with at least 95% identity to SEQ ID NO: 105 or SEQ ID NO: 106,
(v) the nuclease comprises an amino acid sequence with at least 95% identity to SEQ ID NO: 22 and the direct repeat sequence comprises a nucleotide sequence with at least 95% identity to SEQ ID NO: 107 or SEQ ID NO: 108,
(w) the nuclease comprises an amino acid sequence with at least 95% identity to SEQ ID NO: 23 and the direct repeat sequence comprises a nucleotide sequence with at least 95% identity to SEQ ID NO: 109 or SEQ ID NO: 110,
(x) the nuclease comprises an amino acid sequence with at least 95% identity to SEQ ID NO: 24 and the direct repeat sequence comprises a nucleotide sequence with at least 95% identity to SEQ ID NO: 111 or SEQ ID NO: 112,
(y) the nuclease comprises an amino acid sequence with at least 95% identity to SEQ ID NO: 25 and the direct repeat sequence comprises a nucleotide sequence with at least 95% identity to SEQ ID NO: 113 or SEQ ID NO: 114,
(z) the nuclease comprises an amino acid sequence with at least 95% identity to SEQ ID NO: 26 and the direct repeat sequence comprises a nucleotide sequence with at least 95% identity to SEQ ID NO: 115 or SEQ ID NO: 116,
(aa) the nuclease comprises an amino acid sequence with at least 95% identity to SEQ ID NO: 27 and the direct repeat sequence comprises a nucleotide sequence with at least 95% identity to SEQ ID NO: 117 or SEQ ID NO: 118,
(bb) the nuclease comprises an amino acid sequence with at least 95% identity to SEQ ID NO: 28 and the direct repeat sequence comprises a nucleotide sequence with at least 95% identity to SEQ ID NO: 119 or SEQ ID NO: 120,
(cc) the nuclease comprises an amino acid sequence with at least 95% identity to SEQ ID NO: 29 and the direct repeat sequence comprises a nucleotide sequence with at least 95% identity to SEQ ID NO: 121 or SEQ ID NO: 122,
(dd) the nuclease comprises an amino acid sequence with at least 95% identity to SEQ ID NO: 30 and the direct repeat sequence comprises a nucleotide sequence with at least 95% identity to SEQ ID NO: 123 or SEQ ID NO: 124,
(ee) the nuclease comprises an amino acid sequence with at least 95% identity to SEQ ID NO: 31 and the direct repeat sequence comprises a nucleotide sequence with at least 95% identity to SEQ ID NO: 125 or SEQ ID NO: 126; or
(ff) the nuclease comprises an amino acid sequence with at least 95% identity to SEQ ID NO: 32 and the direct repeat sequence comprises a nucleotide sequence with at least 95% identity to SEQ ID NO: 127 or SEQ ID NO: 128.

Embodiment 7. The gene editing system of any one of embodiments 1-6, wherein:
(a) the nuclease comprises an amino acid sequence with at least 95% identity to SEQ ID NO: 1 and the direct repeat sequence comprises the nucleotide sequence set forth in SEQ ID NO: 65 or SEQ ID NO: 66,
(b) the nuclease comprises an amino acid sequence with at least 95% identity to SEQ ID NO: 2 and the direct repeat sequence comprises the nucleotide sequence set forth in SEQ ID NO: 67 or SEQ ID NO: 68,
(c) the nuclease comprises an amino acid sequence with at least 95% identity to SEQ ID NO: 3 and the direct repeat sequence comprises the nucleotide sequence set forth in SEQ ID NO: 69 or SEQ ID NO: 70,
(d) the nuclease comprises an amino acid sequence with at least 95% identity to SEQ ID NO: 4 and the direct repeat sequence comprises the nucleotide sequence set forth in SEQ ID NO: 71 or SEQ ID NO: 72,
(e) the nuclease comprises an amino acid sequence with at least 95% identity to SEQ ID NO: 5 and the direct repeat sequence comprises the nucleotide sequence set forth in SEQ ID NO: 73 or SEQ ID NO: 74,
(f) the nuclease comprises an amino acid sequence with at least 95% identity to SEQ ID NO: 6 and the direct repeat sequence comprises the nucleotide sequence set forth in SEQ ID NO: 75 or SEQ ID NO: 76,
(g) the nuclease comprises an amino acid sequence with at least 95% identity to SEQ ID NO: 7 and the direct repeat sequence comprises the nucleotide sequence set forth in SEQ ID NO: 77 or SEQ ID NO: 78,
(h) the nuclease comprises an amino acid sequence with at least 95% identity to SEQ ID NO: 8 and the direct repeat sequence comprises the nucleotide sequence set forth in SEQ ID NO: 79 or SEQ ID NO: 80,
(i) the nuclease comprises an amino acid sequence with at least 95% identity to SEQ ID NO: 9 and the direct repeat sequence comprises the nucleotide sequence set forth in SEQ ID NO: 81 or SEQ ID NO: 82,
(j) the nuclease comprises an amino acid sequence with at least 95% identity to SEQ ID NO: 10 and the direct repeat sequence comprises the nucleotide sequence set forth in SEQ ID NO: 83 or SEQ ID NO: 84,
(k) the nuclease comprises an amino acid sequence with at least 95% identity to SEQ ID NO: 11 and the direct repeat sequence comprises the nucleotide sequence set forth in SEQ ID NO: 85 or SEQ ID NO: 86,
(l) the nuclease comprises an amino acid sequence with at least 95% identity to SEQ ID NO: 12 and the direct repeat sequence comprises the nucleotide sequence set forth in SEQ ID NO: 87 or SEQ ID NO: 88,
(m) the nuclease comprises an amino acid sequence with at least 95% identity to SEQ ID NO: 13 and the direct repeat sequence comprises the nucleotide sequence set forth in SEQ ID NO: 89 or SEQ ID NO: 90,
(n) the nuclease comprises an amino acid sequence with at least 95% identity to SEQ ID NO: 14 and the direct repeat sequence comprises the nucleotide sequence set forth in SEQ ID NO: 91 or SEQ ID NO: 92,
(o) the nuclease comprises an amino acid sequence with at least 95% identity to SEQ ID NO: 15 and the direct repeat sequence comprises the nucleotide sequence set forth in SEQ ID NO: 93 or SEQ ID NO: 94,
(p) the nuclease comprises an amino acid sequence with at least 95% identity to SEQ ID NO: 16 and the direct repeat sequence comprises the nucleotide sequence set forth in SEQ ID NO: 95 or SEQ ID NO: 96,
(q) the nuclease comprises an amino acid sequence with at least 95% identity to SEQ ID NO: 17 and the direct repeat sequence comprises the nucleotide sequence set forth in SEQ ID NO: 97 or SEQ ID NO: 98,
(r) the nuclease comprises an amino acid sequence with at least 95% identity to SEQ ID NO: 18 and the direct repeat sequence comprises the nucleotide sequence set forth in SEQ ID NO: 99 or SEQ ID NO: 100,
(s) the nuclease comprises an amino acid sequence with at least 95% identity to SEQ ID NO: 19 and the direct repeat sequence comprises the nucleotide sequence set forth in SEQ ID NO: 101 or SEQ ID NO: 102,
(t) the nuclease comprises an amino acid sequence with at least 95% identity to SEQ ID NO: 20 and the direct repeat sequence comprises the nucleotide sequence set forth in SEQ ID NO: 103 or SEQ ID NO: 104,
(u) the nuclease comprises an amino acid sequence with at least 95% identity to SEQ ID NO: 21 and the direct repeat sequence comprises the nucleotide sequence set forth in SEQ ID NO: 105 or SEQ ID NO: 106,
(v) the nuclease comprises an amino acid sequence with at least 95% identity to SEQ ID NO: 22 and the direct repeat sequence comprises the nucleotide sequence set forth in SEQ ID NO: 107 or SEQ ID NO: 108,
(w) the nuclease comprises an amino acid sequence with at least 95% identity to SEQ ID NO: 23 and the direct repeat sequence comprises the nucleotide sequence set forth in SEQ ID NO: 109 or SEQ ID NO: 110,
(x) the nuclease comprises an amino acid sequence with at least 95% identity to SEQ ID NO: 24 and the direct repeat sequence comprises the nucleotide sequence set forth in SEQ ID NO: 111 or SEQ ID NO: 112,
(y) the nuclease comprises an amino acid sequence with at least 95% identity to SEQ ID NO: 25 and the direct repeat sequence comprises the nucleotide sequence set forth in SEQ ID NO: 113 or SEQ ID NO: 114,
(z) the nuclease comprises an amino acid sequence with at least 95% identity to SEQ ID NO: 26 and the direct repeat sequence comprises the nucleotide sequence set forth in SEQ ID NO: 115 or SEQ ID NO: 116,
(aa) the nuclease comprises an amino acid sequence with at least 95% identity to SEQ ID NO: 27 and the direct repeat sequence comprises the nucleotide sequence set forth in SEQ ID NO: 117 or SEQ ID NO: 118,
(bb) the nuclease comprises an amino acid sequence with at least 95% identity to SEQ ID NO: 28 and the direct repeat sequence comprises the nucleotide sequence set forth in SEQ ID NO: 119 or SEQ ID NO: 120,
(cc) the nuclease comprises an amino acid sequence with at least 95% identity to SEQ ID NO: 29 and the direct repeat sequence comprises the nucleotide sequence set forth in SEQ ID NO: 121 or SEQ ID NO: 122,
(dd) the nuclease comprises an amino acid sequence with at least 95% identity to SEQ ID NO: 30 and the direct repeat sequence comprises the nucleotide sequence set forth in SEQ ID NO: 123 or SEQ ID NO: 124,
(ee) the nuclease comprises an amino acid sequence with at least 95% identity to SEQ ID NO: 31 and the direct repeat sequence comprises the nucleotide sequence set forth in SEQ ID NO: 125 or SEQ ID NO: 126; or
(ff) the nuclease comprises an amino acid sequence with at least 95% identity to SEQ ID NO: 32 and the direct repeat sequence comprises the nucleotide sequence set forth in SEQ ID NO: 127 or SEQ ID NO: 128.

Embodiment 8 provides the gene editing system of any one of embodiments 1-7, wherein the RNA guide further comprises a trans-activating crRNA (tracrRNA) sequence.

Embodiment 9 provides the gene editing system of embodiment 8, wherein:
(a) the nuclease comprises an amino acid sequence with at least 95% identity to SEQ ID NO: 1 and the tracrRNA sequence comprises a nucleotide sequence with at least 90% identity to SEQ ID NO: 129,
(b) the nuclease comprises an amino acid sequence with at least 95% identity to SEQ ID NO: 2 and the tracrRNA sequence comprises a nucleotide sequence with at least 90% identity to any one of SEQ ID NOs: 130-137,
(c) the nuclease comprises an amino acid sequence with at least 95% identity to SEQ ID NO: 3 and the tracrRNA sequence comprises a nucleotide sequence with at least 90% identity to any one of SEQ ID NOs: 138-147,
(d) the nuclease comprises an amino acid sequence with at least 95% identity to SEQ ID NO: 4 and the tracrRNA sequence comprises a nucleotide sequence with at least 90% identity to any one of SEQ ID NOs: 148-149,
(e) the nuclease comprises an amino acid sequence with at least 95% identity to SEQ ID NO: 5 and the tracrRNA sequence comprises a nucleotide sequence with at least 90% identity to any one of SEQ ID NOs: 150-163,
(f) the nuclease comprises an amino acid sequence with at least 95% identity to SEQ ID NO: 6 and the tracrRNA sequence comprises a nucleotide sequence with at least 90% identity to any one of SEQ ID NOs: 164-169,
(g) the nuclease comprises an amino acid sequence with at least 95% identity to SEQ ID NO: 7 and the tracrRNA sequence comprises a nucleotide sequence with at least 90% identity to any one of SEQ ID NOs: 170-175,
(h) the nuclease comprises an amino acid sequence with at least 95% identity to SEQ ID NO: 8 and the tracrRNA sequence comprises a nucleotide sequence with at least 90% identity to any one of SEQ ID NOs: 176-177,
(i) the nuclease comprises an amino acid sequence with at least 95% identity to SEQ ID NO: 9 and the tracrRNA sequence comprises a nucleotide sequence with at least 90% identity to any one of SEQ ID NOs: 178-183,
(j) the nuclease comprises an amino acid sequence with at least 95% identity to SEQ ID NO: 10 and the tracrRNA sequence comprises a nucleotide sequence with at least 90% identity to any one of SEQ ID NOs: 184-193,
(k) the nuclease comprises an amino acid sequence with at least 95% identity to SEQ ID NO: 11 and the tracrRNA sequence comprises a nucleotide sequence with at least 90% identity to any one of SEQ ID NOs: 194-199,
(l) the nuclease comprises an amino acid sequence with at least 95% identity to SEQ ID NO: 12 and the tracrRNA sequence comprises a nucleotide sequence with at least 90% identity to any one of SEQ ID NOs: 200-211,
(m) the nuclease comprises an amino acid sequence with at least 95% identity to SEQ ID NO: 13 and the tracrRNA sequence comprises a nucleotide sequence with at least 90% identity to any one of SEQ ID NOs: 212-219,
(n) the nuclease comprises an amino acid sequence with at least 95% identity to SEQ ID NO: 14 and the tracrRNA sequence comprises a nucleotide sequence with at least 90% identity to any one of SEQ ID NOs: 220-227,
(o) the nuclease comprises an amino acid sequence with at least 95% identity to SEQ ID NO: 15 and the tracrRNA sequence comprises a nucleotide sequence with at least 90% identity to any one of SEQ ID NOs: 228-233,
(p) the nuclease comprises an amino acid sequence with at least 95% identity to SEQ ID NO: 16 and the tracrRNA sequence comprises a nucleotide sequence with at least 90% identity to any one of SEQ ID NOs: 234-237,
(q) the nuclease comprises an amino acid sequence with at least 95% identity to SEQ ID NO: 17 and the tracrRNA sequence comprises a nucleotide sequence with at least 90% identity to any one of SEQ ID NOs: 238-243,
(r) the nuclease comprises an amino acid sequence with at least 95% identity to SEQ ID NO: 18 and the tracrRNA sequence comprises a nucleotide sequence with at least 90% identity to any one of SEQ ID NOs: 244-247,
(s) the nuclease comprises an amino acid sequence with at least 95% identity to SEQ ID NO: 19 and the tracrRNA sequence comprises a nucleotide sequence with at least 90% identity to SEQ ID NO: 248,
(t) the nuclease comprises an amino acid sequence with at least 95% identity to SEQ ID NO: 20 and the tracrRNA sequence comprises a nucleotide sequence with at least 90% identity to any one of SEQ ID NOs: 249-254,
(u) the nuclease comprises an amino acid sequence with at least 95% identity to SEQ ID NO: 21 and the tracrRNA sequence comprises a nucleotide sequence with at least 90% identity to any one of SEQ ID NOs: 255-256,
(v) the nuclease comprises an amino acid sequence with at least 95% identity to SEQ ID NO: 22 and the tracrRNA sequence comprises a nucleotide sequence with at least 90% identity to any one of SEQ ID NOs: 257-260,
(w) the nuclease comprises an amino acid sequence with at least 95% identity to SEQ ID NO: 23 and the tracrRNA sequence comprises a nucleotide sequence with at least 90% identity to any one of SEQ ID NOs: 261-264,
(x) the nuclease comprises an amino acid sequence with at least 95% identity to SEQ ID NO: 24 and the tracrRNA sequence comprises a nucleotide sequence with at least 90% identity to any one of SEQ ID NOs: 265-268,
(y) the nuclease comprises an amino acid sequence with at least 95% identity to SEQ ID NO: 25 and the tracrRNA sequence comprises a nucleotide sequence with at least 90% identity to any one of SEQ ID NOs: 269-272,
(z) the nuclease comprises an amino acid sequence with at least 95% identity to SEQ ID NO: 26 and the tracrRNA sequence comprises a nucleotide sequence with at least 90% identity to SEQ ID NO: 273,
(aa) the nuclease comprises an amino acid sequence with at least 95% identity to SEQ ID NO: 27 and the tracrRNA sequence comprises a nucleotide sequence with at least 90% identity to SEQ ID NO: 274,
(bb) the nuclease comprises an amino acid sequence with at least 95% identity to SEQ ID NO: 28 and the tracrRNA sequence comprises a nucleotide sequence with at least 90% identity to SEQ ID NO: 275,
(cc) the nuclease comprises an amino acid sequence with at least 95% identity to SEQ ID NO: 29 and the tracrRNA sequence comprises a nucleotide sequence with at least 90% identity to any one of SEQ ID NOs: 276-281,
(dd) the nuclease comprises an amino acid sequence with at least 95% identity to SEQ ID NO: 30 and the tracrRNA sequence comprises a nucleotide sequence with at least 90% identity to any one of SEQ ID NOs: 282-289,
(ee) the nuclease comprises an amino acid sequence with at least 95% identity to SEQ ID NO: 31 and the tracrRNA sequence comprises a nucleotide sequence with at least 90% identity to any one of SEQ ID NOs: 290-293; or
(ff) the nuclease comprises an amino acid sequence with at least 95% identity to SEQ ID NO: 32 and the tracrRNA sequence comprises a nucleotide sequence with at least 90% identity to any one of SEQ ID NOs: 294-299.

Embodiment 10 provides the gene editing system of embodiment 8 or 9, wherein:
(a) the nuclease comprises an amino acid sequence with at least 95% identity to SEQ ID NO: 1 and the tracrRNA sequence comprises a nucleotide sequence with at least 95% identity to SEQ ID NO: 129,
(b) the nuclease comprises an amino acid sequence with at least 95% identity to SEQ ID NO: 2 and the tracrRNA sequence comprises a nucleotide sequence with at least 95% identity to any one of SEQ ID NOs: 130-137,
(c) the nuclease comprises an amino acid sequence with at least 95% identity to SEQ ID NO: 3 and the tracrRNA sequence comprises a nucleotide sequence with at least 95% identity to any one of SEQ ID NOs: 138-147,
(d) the nuclease comprises an amino acid sequence with at least 95% identity to SEQ ID NO: 4 and the tracrRNA sequence comprises a nucleotide sequence with at least 95% identity to any one of SEQ ID NOs: 148-149,
(e) the nuclease comprises an amino acid sequence with at least 95% identity to SEQ ID NO: 5 and the tracrRNA sequence comprises a nucleotide sequence with at least 95% identity to any one of SEQ ID NOs: 150-163,
(f) the nuclease comprises an amino acid sequence with at least 95% identity to SEQ ID NO: 6 and the tracrRNA sequence comprises a nucleotide sequence with at least 95% identity to any one of SEQ ID NOs: 164-169,
(g) the nuclease comprises an amino acid sequence with at least 95% identity to SEQ ID NO: 7 and the tracrRNA sequence comprises a nucleotide sequence with at least 95% identity to any one of SEQ ID NOs: 170-175,
(h) the nuclease comprises an amino acid sequence with at least 95% identity to SEQ ID NO: 8 and the tracrRNA sequence comprises a nucleotide sequence with at least 95% identity to any one of SEQ ID NOs: 176-177,
(i) the nuclease comprises an amino acid sequence with at least 95% identity to SEQ ID NO: 9 and the tracrRNA sequence comprises a nucleotide sequence with at least 95% identity to any one of SEQ ID NOs: 178-183,
(j) the nuclease comprises an amino acid sequence with at least 95% identity to SEQ ID NO: 10 and the tracrRNA sequence comprises a nucleotide sequence with at least 95% identity to any one of SEQ ID NOs: 184-193,
(k) the nuclease comprises an amino acid sequence with at least 95% identity to SEQ ID NO: 11 and the tracrRNA sequence comprises a nucleotide sequence with at least 95% identity to any one of SEQ ID NOs: 194-199,
(l) the nuclease comprises an amino acid sequence with at least 95% identity to SEQ ID NO: 12 and the tracrRNA sequence comprises a nucleotide sequence with at least 95% identity to any one of SEQ ID NOs: 200-211,
(m) the nuclease comprises an amino acid sequence with at least 95% identity to SEQ ID NO: 13 and the tracrRNA sequence comprises a nucleotide sequence with at least 95% identity to any one of SEQ ID NOs: 212-219,
(n) the nuclease comprises an amino acid sequence with at least 95% identity to SEQ ID NO: 14 and the tracrRNA sequence comprises a nucleotide sequence with at least 95% identity to any one of SEQ ID NOs: 220-227,
(o) the nuclease comprises an amino acid sequence with at least 95% identity to SEQ ID NO: 15 and the tracrRNA sequence comprises a nucleotide sequence with at least 95% identity to any one of SEQ ID NOs: 228-233,
(p) the nuclease comprises an amino acid sequence with at least 95% identity to SEQ ID NO: 16 and the tracrRNA sequence comprises a nucleotide sequence with at least 95% identity to any one of SEQ ID NOs: 234-237,
(q) the nuclease comprises an amino acid sequence with at least 95% identity to SEQ ID NO: 17 and the tracrRNA sequence comprises a nucleotide sequence with at least 95% identity to any one of SEQ ID NOs: 238-243,
(r) the nuclease comprises an amino acid sequence with at least 95% identity to SEQ ID NO: 18 and the tracrRNA sequence comprises a nucleotide sequence with at least 95% identity to any one of SEQ ID NOs: 244-247,
(s) the nuclease comprises an amino acid sequence with at least 95% identity to SEQ ID NO: 19 and the tracrRNA sequence comprises a nucleotide sequence with at least 95% identity to SEQ ID NO: 248,
(t) the nuclease comprises an amino acid sequence with at least 95% identity to SEQ ID NO: 20 and the tracrRNA sequence comprises a nucleotide sequence with at least 95% identity to any one of SEQ ID NOs: 249-254,
(u) the nuclease comprises an amino acid sequence with at least 95% identity to SEQ ID NO: 21 and the tracrRNA sequence comprises a nucleotide sequence with at least 95% identity to any one of SEQ ID NOs: 255-256,
(v) the nuclease comprises an amino acid sequence with at least 95% identity to SEQ ID NO: 22 and the tracrRNA sequence comprises a nucleotide sequence with at least 95% identity to any one of SEQ ID NOs: 257-260,
(w) the nuclease comprises an amino acid sequence with at least 95% identity to SEQ ID NO: 23 and the tracrRNA sequence comprises a nucleotide sequence with at least 95% identity to any one of SEQ ID NOs: 261-264,
(x) the nuclease comprises an amino acid sequence with at least 95% identity to SEQ ID NO: 24 and the tracrRNA sequence comprises a nucleotide sequence with at least 95% identity to any one of SEQ ID NOs: 265-268,
(y) the nuclease comprises an amino acid sequence with at least 95% identity to SEQ ID NO: 25 and the tracrRNA sequence comprises a nucleotide sequence with at least 95% identity to any one of SEQ ID NOs: 269-272,
(z) the nuclease comprises an amino acid sequence with at least 95% identity to SEQ ID NO: 26 and the tracrRNA sequence comprises a nucleotide sequence with at least 95% identity to SEQ ID NO: 273,
(aa) the nuclease comprises an amino acid sequence with at least 95% identity to SEQ ID NO: 27 and the tracrRNA sequence comprises a nucleotide sequence with at least 95% identity to SEQ ID NO: 274,
(bb) the nuclease comprises an amino acid sequence with at least 95% identity to SEQ ID NO: 28 and the tracrRNA sequence comprises a nucleotide sequence with at least 95% identity to SEQ ID NO: 275,
(cc) the nuclease comprises an amino acid sequence with at least 95% identity to SEQ ID NO: 29 and the tracrRNA sequence comprises a nucleotide sequence with at least 95% identity to any one of SEQ ID NOs: 276-281,
(dd) the nuclease comprises an amino acid sequence with at least 95% identity to SEQ ID NO: 30 and the tracrRNA sequence comprises a nucleotide sequence with at least 95% identity to any one of SEQ ID NOs: 282-289,
(ee) the nuclease comprises an amino acid sequence with at least 95% identity to SEQ ID NO: 31 and the tracrRNA sequence comprises a nucleotide sequence with at least 95% identity to any one of SEQ ID NOs: 290-293; or
(ff) the nuclease comprises an amino acid sequence with at least 95% identity to SEQ ID NO: 32 and the tracrRNA sequence comprises a nucleotide sequence with at least 95% identity to any one of SEQ ID NOs: 294-299.

Embodiment 11 provides the gene editing system of any one of embodiments 8-10, wherein:
(a) the nuclease comprises an amino acid sequence with at least 95% identity to SEQ ID NO: 1 and the tracrRNA sequence comprises a nucleotide sequence set forth in SEQ ID NO: 129,
(b) the nuclease comprises an amino acid sequence with at least 95% identity to SEQ ID NO: 2 and the tracrRNA sequence comprises the nucleotide sequence set forth in any one of SEQ ID NOs: 130-137,
(c) the nuclease comprises an amino acid sequence with at least 95% identity to SEQ ID NO: 3 and the tracrRNA sequence comprises the nucleotide sequence set forth in any one of SEQ ID NOs: 138-147,
(d) the nuclease comprises an amino acid sequence with at least 95% identity to SEQ ID NO: 4 and the tracrRNA sequence comprises the nucleotide sequence set forth in any one of SEQ ID NOs: 148-149,
(e) the nuclease comprises an amino acid sequence with at least 95% identity to SEQ ID NO: 5 and the tracrRNA sequence comprises the nucleotide sequence set forth in any one of SEQ ID NOs: 150-163,
(f) the nuclease comprises an amino acid sequence with at least 95% identity to SEQ ID NO: 6 and the tracrRNA sequence comprises the nucleotide sequence set forth in any one of SEQ ID NOs: 164-169,
(g) the nuclease comprises an amino acid sequence with at least 95% identity to SEQ ID NO: 7 and the tracrRNA sequence comprises the nucleotide sequence set forth in any one of SEQ ID NOs: 170-175,
(h) the nuclease comprises an amino acid sequence with at least 95% identity to SEQ ID NO: 8 and the tracrRNA sequence comprises the nucleotide sequence set forth in any one of SEQ ID NOs: 176-177,
(i) the nuclease comprises an amino acid sequence with at least 95% identity to SEQ ID NO: 9 and the tracrRNA sequence comprises the nucleotide sequence set forth in any one of SEQ ID NOs: 178-183,
(j) the nuclease comprises an amino acid sequence with at least 95% identity to SEQ ID NO: 10 and the tracrRNA sequence comprises the nucleotide sequence set forth in any one of SEQ ID NOs: 184-193,
(k) the nuclease comprises an amino acid sequence with at least 95% identity to SEQ ID NO: 11 and the tracrRNA sequence comprises the nucleotide sequence set forth in any one of SEQ ID NOs: 194-199,
(l) the nuclease comprises an amino acid sequence with at least 95% identity to SEQ ID NO: 12 and the tracrRNA sequence comprises the nucleotide sequence set forth in any one of SEQ ID NOs: 200-211,
(m) the nuclease comprises an amino acid sequence with at least 95% identity to SEQ ID NO: 13 and the tracrRNA sequence comprises the nucleotide sequence set forth in any one of SEQ ID NOs: 212-219,
(n) the nuclease comprises an amino acid sequence with at least 95% identity to SEQ ID NO: 14 and the tracrRNA sequence comprises the nucleotide sequence set forth in any one of SEQ ID NOs: 220-227,
(o) the nuclease comprises an amino acid sequence with at least 95% identity to SEQ ID NO: 15 and the tracrRNA sequence comprises the nucleotide sequence set forth in any one of SEQ ID NOs: 228-233,
(p) the nuclease comprises an amino acid sequence with at least 95% identity to SEQ ID NO: 16 and the tracrRNA sequence comprises the nucleotide sequence set forth in any one of SEQ ID NOs: 234-237,
(q) the nuclease comprises an amino acid sequence with at least 95% identity to SEQ ID NO: 17 and the tracrRNA sequence comprises the nucleotide sequence set forth in any one of SEQ ID NOs: 238-243,
(r) the nuclease comprises an amino acid sequence with at least 95% identity to SEQ ID NO: 18 and the tracrRNA sequence comprises the nucleotide sequence set forth in any one of SEQ ID NOs: 244-247,
(s) the nuclease comprises an amino acid sequence with at least 95% identity to SEQ ID NO: 19 and the tracrRNA sequence comprises the nucleotide sequence set forth in SEQ ID NO: 248,
(t) the nuclease comprises an amino acid sequence with at least 95% identity to SEQ ID NO: 20 and the tracrRNA sequence comprises the nucleotide sequence set forth in any one of SEQ ID NOs: 249-254,
(u) the nuclease comprises an amino acid sequence with at least 95% identity to SEQ ID NO: 21 and the tracrRNA sequence comprises the nucleotide sequence set forth in any one of SEQ ID NOs: 255-256,
(v) the nuclease comprises an amino acid sequence with at least 95% identity to SEQ ID NO: 22 and the tracrRNA sequence comprises the nucleotide sequence set forth in any one of SEQ ID NOs: 257-260,
(w) the nuclease comprises an amino acid sequence with at least 95% identity to SEQ ID NO: 23 and the tracrRNA sequence comprises the nucleotide sequence set forth in any one of SEQ ID NOs: 261-264,
(x) the nuclease comprises an amino acid sequence with at least 95% identity to SEQ ID NO: 24 and the tracrRNA sequence comprises the nucleotide sequence set forth in any one of SEQ ID NOs: 265-268,
(y) the nuclease comprises an amino acid sequence with at least 95% identity to SEQ ID NO: 25 and the tracrRNA sequence comprises the nucleotide sequence set forth in any one of SEQ ID NOs: 269-272,
(z) the nuclease comprises an amino acid sequence with at least 95% identity to SEQ ID NO: 26 and the tracrRNA sequence comprises the nucleotide sequence set forth in SEQ ID NO: 273,
(aa) the nuclease comprises an amino acid sequence with at least 95% identity to SEQ ID NO: 27 and the tracrRNA sequence comprises the nucleotide sequence set forth in SEQ ID NO: 274,
(bb) the nuclease comprises an amino acid sequence with at least 95% identity to SEQ ID NO: 28 and the tracrRNA sequence comprises the nucleotide sequence set forth in SEQ ID NO: 275,
(cc) the nuclease comprises an amino acid sequence with at least 95% identity to SEQ ID NO: 29 and the tracrRNA sequence comprises the nucleotide sequence set forth in any one of SEQ ID NOs: 276-281,
(dd) the nuclease comprises an amino acid sequence with at least 95% identity to SEQ ID NO: 30 and the tracrRNA sequence comprises the nucleotide sequence set forth in any one of SEQ ID NOs: 282-289,
(ee) the nuclease comprises an amino acid sequence with at least 95% identity to SEQ ID NO: 31 and the tracrRNA sequence comprises the nucleotide sequence set forth in any one of SEQ ID NOs: 290-293; or
(ff) the nuclease comprises an amino acid sequence with at least 95% identity to SEQ ID NO: 32 and the tracrRNA sequence comprises the nucleotide sequence set forth in any one of SEQ ID NOs: 294-299.

Embodiment 12 provides the gene editing system of any one of embodiments 8-11, wherein the tracrRNA sequence is fused to the direct repeat sequence.

Embodiment 13 provides the gene editing system of any one of embodiments 1-12, wherein the RNA guide is a single molecule RNA guide (sgRNA).

Embodiment 14 provides the gene editing system of embodiment 13, wherein:
(a) the nuclease comprises an amino acid sequence with at least 95% identity to SEQ ID NO: 1 and the sgRNA comprises a nucleotide sequence with at least 90% identity to SEQ ID NO: 300,
(b) the nuclease comprises an amino acid sequence with at least 95% identity to SEQ ID NO: 2 and the sgRNA comprises a nucleotide sequence with at least 90% identity to any one of SEQ ID NOs: 301-308,
(c) the nuclease comprises an amino acid sequence with at least 95% identity to SEQ ID NO: 3 and the sgRNA comprises a nucleotide sequence with at least 90% identity to any one of SEQ ID NOs: 309-318,
(d) the nuclease comprises an amino acid sequence with at least 95% identity to SEQ ID NO: 4 and the sgRNA comprises a nucleotide sequence with at least 90% identity to any one of SEQ ID NOs: 319-320,
(e) the nuclease comprises an amino acid sequence with at least 95% identity to SEQ ID NO: 5 and the sgRNA comprises a nucleotide sequence with at least 90% identity to any one of SEQ ID NOs: 321-334,
(f) the nuclease comprises an amino acid sequence with at least 95% identity to SEQ ID NO: 6 and the sgRNA comprises a nucleotide sequence with at least 90% identity to any one of SEQ ID NOs: 335-340,
(g) the nuclease comprises an amino acid sequence with at least 95% identity to SEQ ID NO: 7 and the sgRNA comprises a nucleotide sequence with at least 90% identity to any one of SEQ ID NOs: 341-346,
(h) the nuclease comprises an amino acid sequence with at least 95% identity to SEQ ID NO: 8 and the sgRNA comprises a nucleotide sequence with at least 90% identity to any one of SEQ ID NOs: 347-348,
(i) the nuclease comprises an amino acid sequence with at least 95% identity to SEQ ID NO: 9 and the sgRNA comprises a nucleotide sequence with at least 90% identity to any one of SEQ ID NOs: 349-354,
(j) the nuclease comprises an amino acid sequence with at least 95% identity to SEQ ID NO: 10 and the sgRNA comprises a nucleotide sequence with at least 90% identity to any one of SEQ ID NOs: 355-364,
(k) the nuclease comprises an amino acid sequence with at least 95% identity to SEQ ID NO: 11 and the sgRNA comprises a nucleotide sequence with at least 90% identity to any one of SEQ ID NOs: 365-370,
(l) the nuclease comprises an amino acid sequence with at least 95% identity to SEQ ID NO: 12 and the sgRNA comprises a nucleotide sequence with at least 90% identity to any one of SEQ ID NOs: 371-382,
(m) the nuclease comprises an amino acid sequence with at least 95% identity to SEQ ID NO: 13 and the sgRNA comprises a nucleotide sequence with at least 90% identity to any one of SEQ ID NOs: 383-390,
(n) the nuclease comprises an amino acid sequence with at least 95% identity to SEQ ID NO: 14 and the sgRNA comprises a nucleotide sequence with at least 90% identity to any one of SEQ ID NOs: 391-398,
(o) the nuclease comprises an amino acid sequence with at least 95% identity to SEQ ID NO: 15 and the sgRNA comprises a nucleotide sequence with at least 90% identity to any one of SEQ ID NOs: 399-404,
(p) the nuclease comprises an amino acid sequence with at least 95% identity to SEQ ID NO: 16 and the sgRNA comprises a nucleotide sequence with at least 90% identity to any one of SEQ ID NOs: 405-408,
(q) the nuclease comprises an amino acid sequence with at least 95% identity to SEQ ID NO: 17 and the sgRNA comprises a nucleotide sequence with at least 90% identity to any one of SEQ ID NOs: 409-414,
(r) the nuclease comprises an amino acid sequence with at least 95% identity to SEQ ID NO: 18 and the sgRNA comprises a nucleotide sequence with at least 90% identity to any one of SEQ ID NOs: 415-418,
(s) the nuclease comprises an amino acid sequence with at least 95% identity to SEQ ID NO: 19 and the sgRNA comprises a nucleotide sequence with at least 90% identity to SEQ ID NO: 419,
(t) the nuclease comprises an amino acid sequence with at least 95% identity to SEQ ID NO: 20 and the sgRNA comprises a nucleotide sequence with at least 90% identity to any one of SEQ ID NOs: 420-425,
(u) the nuclease comprises an amino acid sequence with at least 95% identity to SEQ ID NO: 21 and the sgRNA comprises a nucleotide sequence with at least 90% identity to any one of SEQ ID NOs: 426-427,
(v) the nuclease comprises an amino acid sequence with at least 95% identity to SEQ ID NO: 22 and the sgRNA comprises a nucleotide sequence with at least 90% identity to any one of SEQ ID NOs: 428-431,
(w) the nuclease comprises an amino acid sequence with at least 95% identity to SEQ ID NO: 23 and the sgRNA comprises a nucleotide sequence with at least 90% identity to any one of SEQ ID NOs: 432-435,
(x) the nuclease comprises an amino acid sequence with at least 95% identity to SEQ ID NO: 24 and the sgRNA comprises a nucleotide sequence with at least 90% identity to any one of SEQ ID NOs: 436-439,
(y) the nuclease comprises an amino acid sequence with at least 95% identity to SEQ ID NO: 25 and the sgRNA comprises a nucleotide sequence with at least 90% identity to any one of SEQ ID NOs: 440-443,
(z) the nuclease comprises an amino acid sequence with at least 95% identity to SEQ ID NO: 26 and the sgRNA comprises a nucleotide sequence with at least 90% identity to SEQ ID NO: 444,
(aa) the nuclease comprises an amino acid sequence with at least 95% identity to SEQ ID NO: 27 and the sgRNA comprises a nucleotide sequence with at least 90% identity to SEQ ID NO: 445,
(bb) the nuclease comprises an amino acid sequence with at least 95% identity to SEQ ID NO: 28 and the sgRNA comprises a nucleotide sequence with at least 90% identity to SEQ ID NO: 446,
(cc) the nuclease comprises an amino acid sequence with at least 95% identity to SEQ ID NO: 29 and the sgRNA comprises a nucleotide sequence with at least 90% identity to any one of SEQ ID NOs: 447-452,
(dd) the nuclease comprises an amino acid sequence with at least 95% identity to SEQ ID NO: 30 and the sgRNA comprises a nucleotide sequence with at least 90% identity to any one of SEQ ID NOs: 453-460,
(ee) the nuclease comprises an amino acid sequence with at least 95% identity to SEQ ID NO: 31 and the sgRNA comprises a nucleotide sequence with at least 90% identity to any one of SEQ ID NOs: 461-464; or
(ff) the nuclease comprises an amino acid sequence with at least 95% identity to SEQ ID NO: 32 and the sgRNA comprises a nucleotide sequence with at least 90% identity to any one of SEQ ID NOs: 465-469.

Embodiment 15 provides the gene editing system of embodiment 13 or 14, wherein:
(a) the nuclease comprises an amino acid sequence with at least 95% identity to SEQ ID NO: 1 and the sgRNA comprises a nucleotide sequence with at least 95% identity to SEQ ID NO: 300,
(b) the nuclease comprises an amino acid sequence with at least 95% identity to SEQ ID NO: 2 and the sgRNA comprises a nucleotide sequence with at least 95% identity to any one of SEQ ID NOs: 301-308,
(c) the nuclease comprises an amino acid sequence with at least 95% identity to SEQ ID NO: 3 and the sgRNA comprises a nucleotide sequence with at least 95% identity to any one of SEQ ID NOs: 309-318,
(d) the nuclease comprises an amino acid sequence with at least 95% identity to SEQ ID NO: 4 and the sgRNA comprises a nucleotide sequence with at least 95% identity to any one of SEQ ID NOs: 319-320,
(e) the nuclease comprises an amino acid sequence with at least 95% identity to SEQ ID NO: 5 and the sgRNA comprises a nucleotide sequence with at least 95% identity to any one of SEQ ID NOs: 321-334,
(f) the nuclease comprises an amino acid sequence with at least 95% identity to SEQ ID NO: 6 and the sgRNA comprises a nucleotide sequence with at least 95% identity to any one of SEQ ID NOs: 335-340,
(g) the nuclease comprises an amino acid sequence with at least 95% identity to SEQ ID NO: 7 and the sgRNA comprises a nucleotide sequence with at least 95% identity to any one of SEQ ID NOs: 341-346,
(h) the nuclease comprises an amino acid sequence with at least 95% identity to SEQ ID NO: 8 and the sgRNA comprises a nucleotide sequence with at least 95% identity to any one of SEQ ID NOs: 347-348,
(i) the nuclease comprises an amino acid sequence with at least 95% identity to SEQ ID NO: 9 and the sgRNA comprises a nucleotide sequence with at least 95% identity to any one of SEQ ID NOs: 349-354, the nuclease comprises an amino acid sequence with at least 95% identity to SEQ ID NO: 10 and the sgRNA comprises a nucleotide sequence with at least 95% identity to any one of SEQ ID NOs: 355-364,
(k) the nuclease comprises an amino acid sequence with at least 95% identity to SEQ ID NO: 11 and the sgRNA comprises a nucleotide sequence with at least 95% identity to any one of SEQ ID NOs: 365-370,
(l) the nuclease comprises an amino acid sequence with at least 95% identity to SEQ ID NO: 12 and the sgRNA comprises a nucleotide sequence with at least 95% identity to any one of SEQ ID NOs: 371-382,
(m) the nuclease comprises an amino acid sequence with at least 95% identity to SEQ ID NO: 13 and the sgRNA comprises a nucleotide sequence with at least 95% identity to any one of SEQ ID NOs: 383-390,
(n) the nuclease comprises an amino acid sequence with at least 95% identity to SEQ ID NO: 14 and the sgRNA comprises a nucleotide sequence with at least 95% identity to any one of SEQ ID NOs: 391-398,
(o) the nuclease comprises an amino acid sequence with at least 95% identity to SEQ ID NO: 15 and the sgRNA comprises a nucleotide sequence with at least 95% identity to any one of SEQ ID NOs: 399-404,
(p) the nuclease comprises an amino acid sequence with at least 95% identity to SEQ ID NO: 16 and the sgRNA comprises a nucleotide sequence with at least 95% identity to any one of SEQ ID NOs: 405-408,
(q) the nuclease comprises an amino acid sequence with at least 95% identity to SEQ ID NO: 17 and the sgRNA comprises a nucleotide sequence with at least 95% identity to any one of SEQ ID NOs: 409-414,
(r) the nuclease comprises an amino acid sequence with at least 95% identity to SEQ ID NO: 18 and the sgRNA comprises a nucleotide sequence with at least 95% identity to any one of SEQ ID NOs: 415-418,
(s) the nuclease comprises an amino acid sequence with at least 95% identity to SEQ ID NO: 19 and the sgRNA comprises a nucleotide sequence with at least 95% identity to SEQ ID NO: 419,
(t) the nuclease comprises an amino acid sequence with at least 95% identity to SEQ ID NO: 20 and the sgRNA comprises a nucleotide sequence with at least 95% identity to any one of SEQ ID NOs: 420-425,
(u) the nuclease comprises an amino acid sequence with at least 95% identity to SEQ ID NO: 21 and the sgRNA comprises a nucleotide sequence with at least 95% identity to any one of SEQ ID NOs: 426-427,
(v) the nuclease comprises an amino acid sequence with at least 95% identity to SEQ ID NO: 22 and the sgRNA comprises a nucleotide sequence with at least 95% identity to any one of SEQ ID NOs: 428-431,
(w) the nuclease comprises an amino acid sequence with at least 95% identity to SEQ ID NO: 23 and the sgRNA comprises a nucleotide sequence with at least 95% identity to any one of SEQ ID NOs: 432-435,
(x) the nuclease comprises an amino acid sequence with at least 95% identity to SEQ ID NO: 24 and the sgRNA comprises a nucleotide sequence with at least 95% identity to any one of SEQ ID NOs: 436-439,
(y) the nuclease comprises an amino acid sequence with at least 95% identity to SEQ ID NO: 25 and the sgRNA comprises a nucleotide sequence with at least 95% identity to any one of SEQ ID NOs: 440-443,
(z) the nuclease comprises an amino acid sequence with at least 95% identity to SEQ ID NO: 26 and the sgRNA comprises a nucleotide sequence with at least 95% identity to SEQ ID NO: 444,
(aa) the nuclease comprises an amino acid sequence with at least 95% identity to SEQ ID NO: 27 and the sgRNA comprises a nucleotide sequence with at least 95% identity to SEQ ID NO: 445,
(bb) the nuclease comprises an amino acid sequence with at least 95% identity to SEQ ID NO: 28 and the sgRNA comprises a nucleotide sequence with at least 95% identity to SEQ ID NO: 446,
(cc) the nuclease comprises an amino acid sequence with at least 95% identity to SEQ ID NO: 29 and the sgRNA comprises a nucleotide sequence with at least 95% identity to any one of SEQ ID NOs: 447-452,
(dd) the nuclease comprises an amino acid sequence with at least 95% identity to SEQ ID NO: 30 and the sgRNA comprises a nucleotide sequence with at least 95% identity to any one of SEQ ID NOs: 453-460,
(ee) the nuclease comprises an amino acid sequence with at least 95% identity to SEQ ID NO: 31 and the sgRNA comprises a nucleotide sequence with at least 95% identity to any one of SEQ ID NOs: 461-464; or (ff) the nuclease comprises an amino acid sequence with at least 95% identity to SEQ ID NO: 32 and the sgRNA comprises a nucleotide sequence with at least 95% identity to any one of SEQ ID NOs: 465-469.

Embodiment 16 provides the gene editing system of any one of embodiments 13-15, wherein:
(a) the nuclease comprises an amino acid sequence with at least 95% identity to SEQ ID NO: 1 and the sgRNA comprises the nucleotide sequence set forth in SEQ ID NO: 300,
(b) the nuclease comprises an amino acid sequence with at least 95% identity to SEQ ID NO: 2 and the sgRNA comprises the nucleotide sequence set forth in any one of SEQ ID NOs: 301-308,
(c) the nuclease comprises an amino acid sequence with at least 95% identity to SEQ ID NO: 3 and the sgRNA comprises the nucleotide sequence set forth in any one of SEQ ID NOs: 309-318,
(d) the nuclease comprises an amino acid sequence with at least 95% identity to SEQ ID NO: 4 and the sgRNA comprises the nucleotide sequence set forth in any one of SEQ ID NOs: 319-320,
(e) the nuclease comprises an amino acid sequence with at least 95% identity to SEQ ID NO: 5 and the sgRNA comprises the nucleotide sequence set forth in any one of SEQ ID NOs: 321-334,
(f) the nuclease comprises an amino acid sequence with at least 95% identity to SEQ ID NO: 6 and the sgRNA comprises the nucleotide sequence set forth in any one of SEQ ID NOs: 335-340,
(g) the nuclease comprises an amino acid sequence with at least 95% identity to SEQ ID NO: 7 and the sgRNA comprises the nucleotide sequence set forth in any one of SEQ ID NOs: 341-346,
(h) the nuclease comprises an amino acid sequence with at least 95% identity to SEQ ID NO: 8 and the sgRNA comprises the nucleotide sequence set forth in any one of SEQ ID NOs: 347-348,
(i) the nuclease comprises an amino acid sequence with at least 95% identity to SEQ ID NO: 9 and the sgRNA comprises the nucleotide sequence set forth in any one of SEQ ID NOs: 349-354,
(j) the nuclease comprises an amino acid sequence with at least 95% identity to SEQ ID NO: 10 and the sgRNA comprises the nucleotide sequence set forth in any one of SEQ ID NOs: 355-364,
(k) the nuclease comprises an amino acid sequence with at least 95% identity to SEQ ID NO: 11 and the sgRNA comprises the nucleotide sequence set forth in any one of SEQ ID NOs: 365-370,
(l) the nuclease comprises an amino acid sequence with at least 95% identity to SEQ ID NO: 12 and the sgRNA comprises the nucleotide sequence set forth in any one of SEQ ID NOs: 371-382,
(m) the nuclease comprises an amino acid sequence with at least 95% identity to SEQ ID NO: 13 and the sgRNA comprises the nucleotide sequence set forth in any one of SEQ ID NOs: 383-390,
(n) the nuclease comprises an amino acid sequence with at least 95% identity to SEQ ID NO: 14 and the sgRNA comprises the nucleotide sequence set forth in any one of SEQ ID NOs: 391-398,
(o) the nuclease comprises an amino acid sequence with at least 95% identity to SEQ ID NO: 15 and the sgRNA comprises the nucleotide sequence set forth in any one of SEQ ID NOs: 399-404,
(p) the nuclease comprises an amino acid sequence with at least 95% identity to SEQ ID NO: 16 and the sgRNA comprises the nucleotide sequence set forth in any one of SEQ ID NOs: 405-408,
(q) the nuclease comprises an amino acid sequence with at least 95% identity to SEQ ID NO: 17 and the sgRNA comprises the nucleotide sequence set forth in any one of SEQ ID NOs: 409-414,
(r) the nuclease comprises an amino acid sequence with at least 95% identity to SEQ ID NO: 18 and the sgRNA comprises the nucleotide sequence set forth in any one of SEQ ID NOs: 415-418,
(s) the nuclease comprises an amino acid sequence with at least 95% identity to SEQ ID NO: 19 and the sgRNA comprises the nucleotide sequence set forth in SEQ ID NO: 419,
(t) the nuclease comprises an amino acid sequence with at least 95% identity to SEQ ID NO: 20 and the sgRNA comprises the nucleotide sequence set forth in any one of SEQ ID NOs: 420-425,
(u) the nuclease comprises an amino acid sequence with at least 95% identity to SEQ ID NO: 21 and the sgRNA comprises the nucleotide sequence set forth in any one of SEQ ID NOs: 426-427,
(v) the nuclease comprises an amino acid sequence with at least 95% identity to SEQ ID NO: 22 and the sgRNA comprises the nucleotide sequence set forth in any one of SEQ ID NOs: 428-431,
(w) the nuclease comprises an amino acid sequence with at least 95% identity to SEQ ID NO: 23 and the sgRNA comprises the nucleotide sequence set forth in any one of SEQ ID NOs: 432-435,
(x) the nuclease comprises an amino acid sequence with at least 95% identity to SEQ ID NO: 24 and the sgRNA comprises the nucleotide sequence set forth in any one of SEQ ID NOs: 436-439,
(y) the nuclease comprises an amino acid sequence with at least 95% identity to SEQ ID NO: 25 and the sgRNA comprises the nucleotide sequence set forth in any one of SEQ ID NOs: 440-443,
(z) the nuclease comprises an amino acid sequence with at least 95% identity to SEQ ID NO: 26 and the sgRNA comprises the nucleotide sequence set forth in SEQ ID NO: 444,
(aa) the nuclease comprises an amino acid sequence with at least 95% identity to SEQ ID NO: 27 and the sgRNA comprises the nucleotide sequence set forth in SEQ ID NO: 445,
(bb) the nuclease comprises an amino acid sequence with at least 95% identity to SEQ ID NO: 28 and the sgRNA comprises the nucleotide sequence set forth in SEQ ID NO: 446,
(cc) the nuclease comprises an amino acid sequence with at least 95% identity to SEQ ID NO: 29 and the sgRNA comprises the nucleotide sequence set forth in any one of SEQ ID NOs: 447-452,
(dd) the nuclease comprises an amino acid sequence with at least 95% identity to SEQ ID NO: 30 and the sgRNA comprises the nucleotide sequence set forth in any one of SEQ ID NOs: 453-460,
(ee) the nuclease comprises an amino acid sequence with at least 95% identity to SEQ ID NO: 31 and the sgRNA comprises the nucleotide sequence set forth in any one of SEQ ID NOs: 461-464; or
(ff) the nuclease comprises an amino acid sequence with at least 95% identity to SEQ ID NO: 32 and the sgRNA comprises the nucleotide sequence set forth in any one of SEQ ID NOs: 465-469.

Embodiment 17 provides the gene editing system of any one of embodiments 1-16, wherein the spacer sequence comprises between about 15 nucleotides and about 50 nucleotides in length.

Embodiment 18 provides the gene editing system of any one of embodiments 1-17, wherein the spacer sequence comprises between about 10 nucleotides and about 35 nucleotides in length.

Embodiment 19 provides the gene editing system of any one of embodiments 1-18, wherein the spacer sequence comprises between about 20 nucleotides and about 25 nucleotides in length.

Embodiment 20 provides the gene editing system of any one of embodiments 1-19, wherein the target sequence is adjacent to a protospacer adjacent motif (PAM) sequence.

Embodiment 21 provides the gene editing system of embodiment 20, wherein:
(a) the nuclease comprises an amino acid sequence with at least 95% identity to SEQ ID NO: 1 and the PAM sequence comprises 5′-CC-3′ or 5′-NCC-3′,
(b) the nuclease comprises an amino acid sequence with at least 95% identity to SEQ ID NO: 2 and the PAM sequence comprises 5′-TTC-3′ or 5′-NTTC-3′,
(c) the nuclease comprises an amino acid sequence with at least 95% identity to SEQ ID NO: 3 and the PAM sequence comprises 5′-TY-3′ or 5′-NTY-3′,
(d) the nuclease comprises an amino acid sequence with at least 95% identity to SEQ ID NO: 4 and the PAM sequence comprises 5′-ATC-3′ or 5′-NATC-3′,
(e) the nuclease comprises an amino acid sequence with at least 95% identity to SEQ ID NO: 5 and the PAM sequence comprises 5′-CCN-3′ or 5′-NCCN-3′,
(f) the nuclease comprises an amino acid sequence with at least 95% identity to SEQ ID NO: 6 and the PAM sequence comprises 5′-CCN-3′ or 5′-NCCN-3′,
(g) the nuclease comprises an amino acid sequence with at least 95% identity to SEQ ID NO: 7 and the PAM sequence comprises 5′-CCN-3′ or 5′-NCCN-3′,
(h) the nuclease comprises an amino acid sequence with at least 95% identity to SEQ ID NO: 8 and the PAM sequence comprises 5′-CCN-3′ or 5′-NCCN-3′,
(i) the nuclease comprises an amino acid sequence with at least 95% identity to SEQ ID NO: 9 and the PAM sequence comprises 5′-CYN-3′ or 5′-NCYN-3′,
(j) the nuclease comprises an amino acid sequence with at least 95% identity to SEQ ID NO: 10 and the PAM sequence comprises 5′-CCG-3′ or 5′-NCCG-3′,
(k) the nuclease comprises an amino acid sequence with at least 95% identity to SEQ ID NO: 11 and the PAM sequence comprises 5′-CG-3′ or 5′-NCG-3′,
(l) the nuclease comprises an amino acid sequence with at least 95% identity to SEQ ID NO: 12 and the PAM sequence comprises 5′-A-3′, 5′-NA-3′, or 5′-NAA-3′,
(m) the nuclease comprises an amino acid sequence with at least 95% identity to SEQ ID NO: 13 and the PAM sequence comprises 5′-TA-3′ or 5′-NTA-3′,
(n) the nuclease comprises an amino acid sequence with at least 95% identity to SEQ ID NO: 14 and the PAM sequence comprises 5′-TG-3′ or 5′-NTG-3′,
(o) the nuclease comprises an amino acid sequence with at least 95% identity to SEQ ID NO: 15 and the PAM sequence comprises 5′-C-3′, 5′-NC-3′, or 5′-NNC-3′,
(p) the nuclease comprises an amino acid sequence with at least 95% identity to SEQ ID NO: 16 and the PAM sequence comprises 5′-CC-3′ or 5′-NCC-3′,
(q) the nuclease comprises an amino acid sequence with at least 95% identity to SEQ ID NO: 17 and the PAM sequence comprises 5′-CCNA-3′,
(r) the nuclease comprises an amino acid sequence with at least 95% identity to SEQ ID NO: 18 and the PAM sequence comprises 5′-CG-3′ or 5′-NCG-3′,
(s) the nuclease comprises an amino acid sequence with at least 95% identity to SEQ ID NO: 19 and the PAM sequence comprises 5′-AAN-3′ or 5′-NAAN-3′,
(t) the nuclease comprises an amino acid sequence with at least 95% identity to SEQ ID NO: 20 and the PAM sequence comprises 5′-AAG-3′ or 5′-NAAG-3′,
(u) the nuclease comprises an amino acid sequence with at least 95% identity to SEQ ID NO: 21 and the PAM sequence comprises 5′-AAG-3′ or 5′-NAAG-3′,
(v) the nuclease comprises an amino acid sequence with at least 95% identity to SEQ ID NO: 22 and the PAM sequence comprises 5′-TTN-3′ or 5′-NTTN-3′,
(w) the nuclease comprises an amino acid sequence with at least 95% identity to SEQ ID NO: 23 and the PAM sequence comprises 5′-TTN-3′ or 5′-NTTN-3′,
(x) the nuclease comprises an amino acid sequence with at least 95% identity to SEQ ID NO: 24 and the PAM sequence comprises 5′-GN-3′ or 5′-NGN-3′,
(y) the nuclease comprises an amino acid sequence with at least 95% identity to SEQ ID NO: 25 and the PAM sequence comprises 5′-CT-3′ or 5′-NCT-3′,
(z) the nuclease comprises an amino acid sequence with at least 95% identity to SEQ ID NO: 26 and the PAM sequence comprises 5′-TY-3′ or 5′-NTY-3′,
(aa) the nuclease comprises an amino acid sequence with at least 95% identity to SEQ ID NO: 27 and the PAM sequence comprises 5′-TY-3′ or 5′-NTY-3′,
(bb) the nuclease comprises an amino acid sequence with at least 95% identity to SEQ ID NO: 28 and the PAM sequence comprises 5′-TTC-3′ or 5′-NTTC-3′,
(cc) the nuclease comprises an amino acid sequence with at least 95% identity to SEQ ID NO: 29 and the PAM sequence comprises 5′-TY-3′ or 5′-NTY-3′,
(dd) the nuclease comprises an amino acid sequence with at least 95% identity to SEQ ID NO: 30 and the PAM sequence comprises 5′-GTN-3′ or 5′-NGTN-3′,
(ee) the nuclease comprises an amino acid sequence with at least 95% identity to SEQ ID NO: 31 and the PAM sequence comprises 5′-CCN-3′ or 5′-NCCN-3′,
(ff) the nuclease comprises an amino acid sequence with at least 95% identity to SEQ ID NO: 32 and the PAM sequence comprises 5′-CCY-3′ or 5′-NCCY-3′, wherein N is any nucleotide and Y is cytosine or thymine.

Embodiment 22 provides the gene editing system of any one of embodiments 1-21, wherein the nuclease comprises the amino acid sequence set forth in any one of SEQ ID NOs: 1-32.

Embodiment 23 provides the gene editing system of any one of embodiments 1-22, wherein the nuclease further comprises a peptide tag, a fluorescent protein, a base-editing domain, a DNA methylation domain, a histone residue modification domain, a localization factor, a transcription modification factor, a light-gated control factor, a chemically inducible factor, or a chromatin visualization factor.

Embodiment 24 provides the gene editing system of any one of embodiments 1-23, which comprises the first nucleic acid encoding the nuclease polypeptide.

Embodiment 25 provides the gene editing system of embodiment 24, wherein the first nucleic acid is codon-optimized for expression in a cell.

Embodiment 26 provides the gene editing system of embodiment 24 or 25, wherein the first nucleic acid is a messenger RNA (mRNA).

Embodiment 27 provides the gene editing system of any one of embodiments 24-26, wherein the first nucleic acid is included in a vector.

Embodiment 28 provides the gene editing system of any one of embodiments 1-27, wherein the system comprises the second nucleic acid encoding the RNA guide.

Embodiment 29 provides the gene editing system of embodiment 28, wherein the nucleic acid encoding the RNA guide is located in a vector.

Embodiment 30 provides the gene editing system of any one of embodiments 27-29, wherein the vector comprises the both the first nucleic acid encoding the nuclease polypeptide and the second nucleic acid encoding the RNA guide.

Embodiment 31 provides the gene editing system of any one of embodiments 1-30, wherein the system comprises the first nucleic acid encoding the nuclease polypeptide, which is located on a first vector, and wherein the system comprises the second nucleic acid encoding the RNA guide, which is located on a second vector.

Embodiment 32 provides the gene editing system of embodiment 31, wherein the first and second vector are the same vector.

Embodiment 33 provides the gene editing system of any one of embodiments 27-32, wherein the vector comprises a retroviral vector, a lentiviral vector, a phage vector, an adenoviral vector, an adeno-associated vector, or a herpes simplex vector.

Embodiment 34 provides the gene editing system of any one of embodiments 1-33, wherein the gene editing system is present in a delivery gene editing system comprising a nanoparticle, a liposome, an exosome, a microvesicle, or a gene-gun.

Embodiment 35 provides a cell comprising the gene editing system of any one of embodiments 1-34.

Embodiment 36 provides the cell of embodiment 35, wherein the cell is a eukaryotic cell.

Embodiment 37 provides the cell of embodiment 35 or 36, wherein the cell is a mammalian cell or a plant cell.

Embodiment 38 provides the cell of any one of embodiments 35-37, wherein the cell is a human cell.

Embodiment 39 provides a method of introducing an indel into a target nucleic acid in a cell comprising:
(a) providing the gene editing system of any one of embodiments 1-38; and
(b) delivering the gene editing system to the cell,
wherein recognition of the target sequence by the gene editing system results in a modification of the target nucleic acid.

Embodiment 40 provides the method of embodiment 39, wherein delivering the gene editing system to the cell is by transfection.

Embodiment 41 provides the method of embodiment 39 or 40, wherein the cell is a eukaryotic cell.

Embodiment 42 provides the method of any one of embodiments 39-41, wherein the cell is a human cell.

Embodiment 43 provides the gene editing system of embodiment 1, wherein the nuclease comprises an amino acid sequence with at least 80% identity to SEQ ID NO: 26 or 27.

Embodiment 44 provides the gene editing system of embodiment 43, wherein the nuclease comprises a RuvC domain or a split RuvC domain.

Embodiment 45 provides the gene editing system of embodiment 43 or 44, wherein the nuclease comprises a catalytic residue (e.g., aspartic acid or glutamic acid).

Embodiment 46 provides the gene editing system of any one of embodiments 43-45, wherein the nuclease comprises an amino acid sequence with at least 95% identity to SEQ ID NO: 26 or 27.

Embodiment 47 provides the gene editing system of any one of embodiments 43-46, wherein:
(z) the nuclease comprises an amino acid sequence with at least 95% identity to SEQ ID NO: 26 and the direct repeat sequence comprises a nucleotide sequence with at least 90% identity to SEQ ID NO: 115 or SEQ ID NO: 116, or
(aa) the nuclease comprises an amino acid sequence with at least 95% identity to SEQ ID NO: 27 and the direct repeat sequence comprises a nucleotide sequence with at least 90% identity to SEQ ID NO: 117 or SEQ ID NO: 118.

Embodiment 48 provides the gene editing system of any one of embodiments 43-47, wherein:
(z) the nuclease comprises an amino acid sequence with at least 95% identity to SEQ ID NO: 26 and the direct repeat sequence comprises a nucleotide sequence with at least 95% identity to SEQ ID NO: 115 or SEQ ID NO: 116, or
(aa) the nuclease comprises an amino acid sequence with at least 95% identity to SEQ ID NO: 27 and the direct repeat sequence comprises a nucleotide sequence with at least 95% identity to SEQ ID NO: 117 or SEQ ID NO: 118.

Embodiment 49 provides the gene editing system of any one of embodiments 43-48, wherein:
(z) the nuclease comprises an amino acid sequence with at least 95% identity to SEQ ID NO: 26 and the direct repeat sequence comprises the nucleotide sequence set forth in SEQ ID NO: 115 or SEQ ID NO: 116, or
(aa) the nuclease comprises an amino acid sequence with at least 95% identity to SEQ ID NO: 27 and the direct repeat sequence comprises the nucleotide sequence set forth in SEQ ID NO: 117 or SEQ ID NO: 118.

Embodiment 50 provides the gene editing system of any one of embodiments 43-49, wherein the RNA guide further comprises a trans-activating crRNA (tracrRNA) sequence.

Embodiment 51 provides the gene editing system of embodiment 50, wherein:
(z) the nuclease comprises an amino acid sequence with at least 95% identity to SEQ ID NO: 26 and the tracrRNA sequence comprises a nucleotide sequence with at least 90% identity to SEQ ID NO: 273, or
(aa) the nuclease comprises an amino acid sequence with at least 95% identity to SEQ ID NO: 27 and the tracrRNA sequence comprises a nucleotide sequence with at least 90% identity to SEQ ID NO: 274.

Embodiment 52 provides the gene editing system of embodiment 50 or 51, wherein:
(z) the nuclease comprises an amino acid sequence with at least 95% identity to SEQ ID NO: 26 and the tracrRNA sequence comprises a nucleotide sequence with at least 95% identity to SEQ ID NO: 273, or
(aa) the nuclease comprises an amino acid sequence with at least 95% identity to SEQ ID NO: 27 and the tracrRNA sequence comprises a nucleotide sequence with at least 95% identity to SEQ ID NO: 274.

Embodiment 53 provides the gene editing system of any one of embodiments 50-52, wherein:
(z) the nuclease comprises an amino acid sequence with at least 95% identity to SEQ ID NO: 26 and the tracrRNA sequence comprises the nucleotide sequence set forth in SEQ ID NO: 273, or
(aa) the nuclease comprises an amino acid sequence with at least 95% identity to SEQ ID NO: 27 and the tracrRNA sequence comprises the nucleotide sequence set forth in SEQ ID NO: 274.

Embodiment 54 provides the gene editing system of any one of embodiments 50-53, wherein the tracrRNA sequence is fused to the direct repeat sequence.

Embodiment provides the gene editing system of any one of embodiments 43-54, wherein the RNA guide is a single molecule RNA guide (sgRNA).

Embodiment 56 provides the gene editing system of embodiment 55, wherein:
(z) the nuclease comprises an amino acid sequence with at least 95% identity to SEQ ID NO: 26 and the sgRNA comprises a nucleotide sequence with at least 90% identity to SEQ ID NO: 444, or
(aa) the nuclease comprises an amino acid sequence with at least 95% identity to SEQ ID NO: 27 and the sgRNA comprises a nucleotide sequence with at least 90% identity to SEQ ID NO: 445.

Embodiment 57 provides the gene editing system of embodiment 55 or 56, wherein:
(z) the nuclease comprises an amino acid sequence with at least 95% identity to SEQ ID NO: 26 and the sgRNA comprises a nucleotide sequence with at least 95% identity to SEQ ID NO: 444, or
(aa) the nuclease comprises an amino acid sequence with at least 95% identity to SEQ ID NO: 27 and the sgRNA comprises a nucleotide sequence with at least 95% identity to SEQ ID NO: 445.

Embodiment 58 provides the gene editing system of any one of embodiments 55-57, wherein:
(z) the nuclease comprises an amino acid sequence with at least 95% identity to SEQ ID NO: 26 and the sgRNA comprises the nucleotide sequence set forth in SEQ ID NO: 444, or
(aa) the nuclease comprises an amino acid sequence with at least 95% identity to SEQ ID NO: 27 and the sgRNA comprises the nucleotide sequence set forth in SEQ ID NO: 445.

Embodiment 59 provides the gene editing system of any one of embodiments 43-58, wherein the spacer sequence comprises between about 15 nucleotides and about 50 nucleotides in length.

Embodiment 60 provides the gene editing system of any one of embodiments 43-59, wherein the spacer sequence comprises between about 10 nucleotides and about 35 nucleotides in length.

Embodiment 61 provides the gene editing system of any one of embodiments 43-60, wherein the spacer sequence comprises between about 20 nucleotides and about 25 nucleotides in length.

Embodiment 62 provides the gene editing system of any one of embodiments 43-61, wherein the target sequence is adjacent to a protospacer adjacent motif (PAM) sequence.

Embodiment 63 provides the gene editing system of embodiment 62, wherein:
(z) the nuclease comprises an amino acid sequence with at least 95% identity to SEQ ID NO: 26 and the PAM sequence comprises 5′-TY-3′ or 5′-NTY-3′, or
(aa) the nuclease comprises an amino acid sequence with at least 95% identity to SEQ ID NO: 27 and the PAM sequence comprises 5′-TY-3′ or 5′-NTY-3′.

Embodiment 64 provides the gene editing system of any one of embodiments 43-63, wherein the nuclease comprises the amino acid sequence set forth in SEQ ID NO: 26 or 27.

Embodiment 65 provides the gene editing system of any one of embodiments 43-64, wherein the nuclease further comprises a peptide tag, a fluorescent protein, a base-editing domain, a DNA methylation domain, a histone residue modification domain, a localization factor, a transcription modification factor, a light-gated control factor, a chemically inducible factor, or a chromatin visualization factor.

Embodiment 66 provides the gene editing system of any one of embodiments 43-65, which comprises the first nucleic acid encoding the nuclease polypeptide.

Embodiment 67 provides the gene editing system of embodiment 66, wherein the first nucleic acid is codon-optimized for expression in a cell.

Embodiment 68 provides the gene editing system of embodiment 66 or 67, wherein the first nucleic acid is a messenger RNA (mRNA).

Embodiment 69 provides the gene editing system of any one of embodiments 66-68, wherein the first nucleic acid is included in a vector.

Embodiment 70 provides the gene editing system of any one of embodiments 43-69, wherein the system comprises the second nucleic acid encoding the RNA guide.

Embodiment 71 provides the gene editing system of embodiment 70, wherein the nucleic acid encoding the RNA guide is located in a vector.

Embodiment 72 provides the gene editing system of any one of embodiments 69-71, wherein the vector comprises the both the first nucleic acid encoding the nuclease polypeptide and the second nucleic acid encoding the RNA guide.

Embodiment 73 provides the gene editing system of any one of embodiments 43-72, wherein the system comprises the first nucleic acid encoding the nuclease polypeptide, which is located on a first vector, and wherein the system comprises the second nucleic acid encoding the RNA guide, which is located on a second vector.

Embodiment 74 provides the gene editing system of embodiment 73, wherein the first and second vector are the same vector.

Embodiment 75 provides the gene editing system of any one of embodiments 69-74, wherein the vector comprises a retroviral vector, a lentiviral vector, a phage vector, an adenoviral vector, an adeno-associated vector, or a herpes simplex vector.

Embodiment 76 provides the gene editing system of any one of embodiments 43-75, wherein the gene editing system is present in a delivery gene editing system comprising a nanoparticle, a liposome, an exosome, a microvesicle, or a gene-gun.

Embodiment 77 provides a cell comprising the gene editing system of any one of embodiments 43-76.

Embodiment 78 provides the cell of embodiment 77, wherein the cell is a eukaryotic cell.

Embodiment 79 provides the cell of embodiment 77 or 78, wherein the cell is a mammalian cell or a plant cell.

Embodiment 80 provides the cell of any one of embodiments 77-79, wherein the cell is a human cell.

Embodiment 81 provides a method of introducing an indel into a target nucleic acid in a cell comprising:
(a) providing the gene editing system of any one of embodiments 43-80; and
(b) delivering the gene editing system to the cell, wherein recognition of the target sequence by the gene editing system results in a modification of the target nucleic acid.

Embodiment 82 provides the method of embodiment 81, wherein delivering the gene editing system to the cell is by transfection.

Embodiment 83 provides the method of embodiment 81 or 82, wherein the cell is a eukaryotic cell.

Embodiment 84 provides the method of any one of embodiments 81-83, wherein the cell is a human cell.

1. A gene editing system comprising:
(a) a nuclease or a first nucleic acid encoding the nuclease, wherein the nuclease comprises an amino acid sequence with at least 80% identity to any one of SEQ ID NOs: 1-32; and
(b) an RNA guide or a second nucleic acid encoding the RNA guide, wherein the RNA guide comprises a direct repeat sequence and a spacer sequence,
wherein the nuclease binds to the RNA guide, and wherein the spacer sequence is specific to a target sequence within a target nucleic acid.

2. The gene editing system of claim 1, wherein the nuclease comprises a RuvC domain or a split RuvC domain.

3. The gene editing system of claim 1, wherein the nuclease comprises a catalytic residue (e.g., aspartic acid or glutamic acid).

4. The gene editing system of claim 1, wherein the nuclease comprises an amino acid sequence with at least 95% identity to any one of SEQ ID NOs: 1-32.

5. The gene editing system of claim 1, wherein:
(a) the nuclease comprises an amino acid sequence with at least 95% identity to SEQ ID NO: 1 and the direct repeat sequence comprises a nucleotide sequence with at least 90% identity to SEQ ID NO: 65 or SEQ ID NO: 66,
(b) the nuclease comprises an amino acid sequence with at least 95% identity to SEQ ID NO: 2 and the direct repeat sequence comprises a nucleotide sequence with at least 90% identity to SEQ ID NO: 67 or SEQ ID NO: 68,
(c) the nuclease comprises an amino acid sequence with at least 95% identity to SEQ ID NO: 3 and the direct repeat sequence comprises a nucleotide sequence with at least 90% identity to SEQ ID NO: 69 or SEQ ID NO: 70,
(d) the nuclease comprises an amino acid sequence with at least 95% identity to SEQ ID NO: 4 and the direct repeat sequence comprises a nucleotide sequence with at least 90% identity to SEQ ID NO: 71 or SEQ ID NO: 72,
(e) the nuclease comprises an amino acid sequence with at least 95% identity to SEQ ID NO: 5 and the direct repeat sequence comprises a nucleotide sequence with at least 90% identity to SEQ ID NO: 73 or SEQ ID NO: 74,
(f) the nuclease comprises an amino acid sequence with at least 95% identity to SEQ ID NO: 6 and the direct repeat sequence comprises a nucleotide sequence with at least 90% identity to SEQ ID NO: 75 or SEQ ID NO: 76,
(g) the nuclease comprises an amino acid sequence with at least 95% identity to SEQ ID NO: 7 and the direct repeat sequence comprises a nucleotide sequence with at least 90% identity to SEQ ID NO: 77 or SEQ ID NO: 78,
(h) the nuclease comprises an amino acid sequence with at least 95% identity to SEQ ID NO: 8 and the direct repeat sequence comprises a nucleotide sequence with at least 90% identity to SEQ ID NO: 79 or SEQ ID NO: 80,
(i) the nuclease comprises an amino acid sequence with at least 95% identity to SEQ ID NO: 9 and the direct repeat sequence comprises a nucleotide sequence with at least 90% identity to SEQ ID NO: 81 or SEQ ID NO: 82,
(j) the nuclease comprises an amino acid sequence with at least 95% identity to SEQ ID NO: 10 and the direct repeat sequence comprises a nucleotide sequence with at least 90% identity to SEQ ID NO: 83 or SEQ ID NO: 84,
(k) the nuclease comprises an amino acid sequence with at least 95% identity to SEQ ID NO: 11 and the direct repeat sequence comprises a nucleotide sequence with at least 90% identity to SEQ ID NO: 85 or SEQ ID NO: 86,
(l) the nuclease comprises an amino acid sequence with at least 95% identity to SEQ ID NO: 12 and the direct repeat sequence comprises a nucleotide sequence with at least 90% identity to SEQ ID NO: 87 or SEQ ID NO: 88,
(m) the nuclease comprises an amino acid sequence with at least 95% identity to SEQ ID NO: 13 and the direct repeat sequence comprises a nucleotide sequence with at least 90% identity to SEQ ID NO: 89 or SEQ ID NO: 90,
(n) the nuclease comprises an amino acid sequence with at least 95% identity to SEQ ID NO: 14 and the direct repeat sequence comprises a nucleotide sequence with at least 90% identity to SEQ ID NO: 91 or SEQ ID NO: 92,
(o) the nuclease comprises an amino acid sequence with at least 95% identity to SEQ ID NO: 15 and the direct repeat sequence comprises a nucleotide sequence with at least 90% identity to SEQ ID NO: 93 or SEQ ID NO: 94,
(p) the nuclease comprises an amino acid sequence with at least 95% identity to SEQ ID NO: 16 and the direct repeat sequence comprises a nucleotide sequence with at least 90% identity to SEQ ID NO: 95 or SEQ ID NO: 96,
(q) the nuclease comprises an amino acid sequence with at least 95% identity to SEQ ID NO: 17 and the direct repeat sequence comprises a nucleotide sequence with at least 90% identity to SEQ ID NO: 97 or SEQ ID NO: 98,
(r) the nuclease comprises an amino acid sequence with at least 95% identity to SEQ ID NO: 18 and the direct repeat sequence comprises a nucleotide sequence with at least 90% identity to SEQ ID NO: 99 or SEQ ID NO: 100,
(s) the nuclease comprises an amino acid sequence with at least 95% identity to SEQ ID NO: 19 and the direct repeat sequence comprises a nucleotide sequence with at least 90% identity to SEQ ID NO: 101 or SEQ ID NO: 102,
(t) the nuclease comprises an amino acid sequence with at least 95% identity to SEQ ID NO: 20 and the direct repeat sequence comprises a nucleotide sequence with at least 90% identity to SEQ ID NO: 103 or SEQ ID NO: 104,
(u) the nuclease comprises an amino acid sequence with at least 95% identity to SEQ ID NO: 21 and the direct repeat sequence comprises a nucleotide sequence with at least 90% identity to SEQ ID NO: 105 or SEQ ID NO: 106,
(v) the nuclease comprises an amino acid sequence with at least 95% identity to SEQ ID NO: 22 and the direct repeat sequence comprises a nucleotide sequence with at least 90% identity to SEQ ID NO: 107 or SEQ ID NO: 108,
(w) the nuclease comprises an amino acid sequence with at least 95% identity to SEQ ID NO: 23 and the direct repeat sequence comprises a nucleotide sequence with at least 90% identity to SEQ ID NO: 109 or SEQ ID NO: 110,
(x) the nuclease comprises an amino acid sequence with at least 95% identity to SEQ ID NO: 24 and the direct repeat sequence comprises a nucleotide sequence with at least 90% identity to SEQ ID NO: 111 or SEQ ID NO: 112,
(y) the nuclease comprises an amino acid sequence with at least 95% identity to SEQ ID NO: 25 and the direct repeat sequence comprises a nucleotide sequence with at least 90% identity to SEQ ID NO: 113 or SEQ ID NO: 114,
(z) the nuclease comprises an amino acid sequence with at least 95% identity to SEQ ID NO: 26 and the direct repeat sequence comprises a nucleotide sequence with at least 90% identity to SEQ ID NO: 115 or SEQ ID NO: 116,
(aa) the nuclease comprises an amino acid sequence with at least 95% identity to SEQ ID NO: 27 and the direct repeat sequence comprises a nucleotide sequence with at least 90% identity to SEQ ID NO: 117 or SEQ ID NO: 118,
(bb) the nuclease comprises an amino acid sequence with at least 95% identity to SEQ ID NO: 28 and the direct repeat sequence comprises a nucleotide sequence with at least 90% identity to SEQ ID NO: 119 or SEQ ID NO: 120,
(cc) the nuclease comprises an amino acid sequence with at least 95% identity to SEQ ID NO: 29 and the direct repeat sequence comprises a nucleotide sequence with at least 90% identity to SEQ ID NO: 121 or SEQ ID NO: 122,
(dd) the nuclease comprises an amino acid sequence with at least 95% identity to SEQ ID NO: 30 and the direct repeat sequence comprises a nucleotide sequence with at least 90% identity to SEQ ID NO: 123 or SEQ ID NO: 124,
(ee) the nuclease comprises an amino acid sequence with at least 95% identity to SEQ ID NO: 31 and the direct repeat sequence comprises a nucleotide sequence with at least 90% identity to SEQ ID NO: 125 or SEQ ID NO: 126; or
(ff) the nuclease comprises an amino acid sequence with at least 95% identity to SEQ ID NO: 32 and the direct repeat sequence comprises a nucleotide sequence with at least 90% identity to SEQ ID NO: 127 or SEQ ID NO: 128.

6. The gene editing system of claim 1, wherein:
(a) the nuclease comprises an amino acid sequence with at least 95% identity to SEQ ID NO: 1 and the direct repeat sequence comprises a nucleotide sequence with at least 95% identity to SEQ ID NO: 65 or SEQ ID NO: 66,
(b) the nuclease comprises an amino acid sequence with at least 95% identity to SEQ ID NO: 2 and the direct repeat sequence comprises a nucleotide sequence with at least 95% identity to SEQ ID NO: 67 or SEQ ID NO: 68,
(c) the nuclease comprises an amino acid sequence with at least 95% identity to SEQ ID NO: 3 and the direct repeat sequence comprises a nucleotide sequence with at least 95% identity to SEQ ID NO: 69 or SEQ ID NO: 70,
(d) the nuclease comprises an amino acid sequence with at least 95% identity to SEQ ID NO: 4 and the direct repeat sequence comprises a nucleotide sequence with at least 95% identity to SEQ ID NO: 71 or SEQ ID NO: 72,
(e) the nuclease comprises an amino acid sequence with at least 95% identity to SEQ ID NO: 5 and the direct repeat sequence comprises a nucleotide sequence with at least 95% identity to SEQ ID NO: 73 or SEQ ID NO: 74,
(f) the nuclease comprises an amino acid sequence with at least 95% identity to SEQ ID NO: 6 and the direct repeat sequence comprises a nucleotide sequence with at least 95% identity to SEQ ID NO: 75 or SEQ ID NO: 76,
(g) the nuclease comprises an amino acid sequence with at least 95% identity to SEQ ID NO: 7 and the direct repeat sequence comprises a nucleotide sequence with at least 95% identity to SEQ ID NO: 77 or SEQ ID NO: 78,
(h) the nuclease comprises an amino acid sequence with at least 95% identity to SEQ ID NO: 8 and the direct repeat sequence comprises a nucleotide sequence with at least 95% identity to SEQ ID NO: 79 or SEQ ID NO: 80,
(i) the nuclease comprises an amino acid sequence with at least 95% identity to SEQ ID NO: 9 and the direct repeat sequence comprises a nucleotide sequence with at least 95% identity to SEQ ID NO: 81 or SEQ ID NO: 82,
(j) the nuclease comprises an amino acid sequence with at least 95% identity to SEQ ID NO: 10 and the direct repeat sequence comprises a nucleotide sequence with at least 95% identity to SEQ ID NO: 83 or SEQ ID NO: 84,
(k) the nuclease comprises an amino acid sequence with at least 95% identity to SEQ ID NO: 11 and the direct repeat sequence comprises a nucleotide sequence with at least 95% identity to SEQ ID NO: 85 or SEQ ID NO: 86,
(l) the nuclease comprises an amino acid sequence with at least 95% identity to SEQ ID NO: 12 and the direct repeat sequence comprises a nucleotide sequence with at least 95% identity to SEQ ID NO: 87 or SEQ ID NO: 88,
(m) the nuclease comprises an amino acid sequence with at least 95% identity to SEQ ID NO: 13 and the direct repeat sequence comprises a nucleotide sequence with at least 95% identity to SEQ ID NO: 89 or SEQ ID NO: 90,
(n) the nuclease comprises an amino acid sequence with at least 95% identity to SEQ ID NO: 14 and the direct repeat sequence comprises a nucleotide sequence with at least 95% identity to SEQ ID NO: 91 or SEQ ID NO: 92,
(o) the nuclease comprises an amino acid sequence with at least 95% identity to SEQ ID NO: 15 and the direct repeat sequence comprises a nucleotide sequence with at least 95% identity to SEQ ID NO: 93 or SEQ ID NO: 94,
(p) the nuclease comprises an amino acid sequence with at least 95% identity to SEQ ID NO: 16 and the direct repeat sequence comprises a nucleotide sequence with at least 95% identity to SEQ ID NO: 95 or SEQ ID NO: 96,
(q) the nuclease comprises an amino acid sequence with at least 95% identity to SEQ ID NO: 17 and the direct repeat sequence comprises a nucleotide sequence with at least 95% identity to SEQ ID NO: 97 or SEQ ID NO: 98,
(r) the nuclease comprises an amino acid sequence with at least 95% identity to SEQ ID NO: 18 and the direct repeat sequence comprises a nucleotide sequence with at least 95% identity to SEQ ID NO: 99 or SEQ ID NO: 100,
(s) the nuclease comprises an amino acid sequence with at least 95% identity to SEQ ID NO: 19 and the direct repeat sequence comprises a nucleotide sequence with at least 95% identity to SEQ ID NO: 101 or SEQ ID NO: 102,
(t) the nuclease comprises an amino acid sequence with at least 95% identity to SEQ ID NO: 20 and the direct repeat sequence comprises a nucleotide sequence with at least 95% identity to SEQ ID NO: 103 or SEQ ID NO: 104,
(u) the nuclease comprises an amino acid sequence with at least 95% identity to SEQ ID NO: 21 and the direct repeat sequence comprises a nucleotide sequence with at least 95% identity to SEQ ID NO: 105 or SEQ ID NO: 106,
(v) the nuclease comprises an amino acid sequence with at least 95% identity to SEQ ID NO: 22 and the direct repeat sequence comprises a nucleotide sequence with at least 95% identity to SEQ ID NO: 107 or SEQ ID NO: 108,
(w) the nuclease comprises an amino acid sequence with at least 95% identity to SEQ ID NO: 23 and the direct repeat sequence comprises a nucleotide sequence with at least 95% identity to SEQ ID NO: 109 or SEQ ID NO: 110,
(x) the nuclease comprises an amino acid sequence with at least 95% identity to SEQ ID NO: 24 and the direct repeat sequence comprises a nucleotide sequence with at least 95% identity to SEQ ID NO: 111 or SEQ ID NO: 112,
(y) the nuclease comprises an amino acid sequence with at least 95% identity to SEQ ID NO: 25 and the direct repeat sequence comprises a nucleotide sequence with at least 95% identity to SEQ ID NO: 113 or SEQ ID NO: 114,
(z) the nuclease comprises an amino acid sequence with at least 95% identity to SEQ ID NO: 26 and the direct repeat sequence comprises a nucleotide sequence with at least 95% identity to SEQ ID NO: 115 or SEQ ID NO: 116,
(aa) the nuclease comprises an amino acid sequence with at least 95% identity to SEQ ID NO: 27 and the direct repeat sequence comprises a nucleotide sequence with at least 95% identity to SEQ ID NO: 117 or SEQ ID NO: 118,
(bb) the nuclease comprises an amino acid sequence with at least 95% identity to SEQ ID NO: 28 and the direct repeat sequence comprises a nucleotide sequence with at least 95% identity to SEQ ID NO: 119 or SEQ ID NO: 120,
(cc) the nuclease comprises an amino acid sequence with at least 95% identity to SEQ ID NO: 29 and the direct repeat sequence comprises a nucleotide sequence with at least 95% identity to SEQ ID NO: 121 or SEQ ID NO: 122,
(dd) the nuclease comprises an amino acid sequence with at least 95% identity to SEQ ID NO: 30 and the direct repeat sequence comprises a nucleotide sequence with at least 95% identity to SEQ ID NO: 123 or SEQ ID NO: 124,
(ee) the nuclease comprises an amino acid sequence with at least 95% identity to SEQ ID NO: 31 and the direct repeat sequence comprises a nucleotide sequence with at least 95% identity to SEQ ID NO: 125 or SEQ ID NO: 126; or
(ff) the nuclease comprises an amino acid sequence with at least 95% identity to SEQ ID NO: 32 and the direct repeat sequence comprises a nucleotide sequence with at least 95% identity to SEQ ID NO: 127 or SEQ ID NO: 128.

7. The gene editing system of claim 1, wherein:
(a) the nuclease comprises an amino acid sequence with at least 95% identity to SEQ ID NO: 1 and the direct repeat sequence comprises the nucleotide sequence set forth in SEQ ID NO: 65 or SEQ ID NO: 66,
(b) the nuclease comprises an amino acid sequence with at least 95% identity to SEQ ID NO: 2 and the direct repeat sequence comprises the nucleotide sequence set forth in SEQ ID NO: 67 or SEQ ID NO: 68,
(c) the nuclease comprises an amino acid sequence with at least 95% identity to SEQ ID NO: 3 and the direct repeat sequence comprises the nucleotide sequence set forth in SEQ ID NO: 69 or SEQ ID NO: 70,
(d) the nuclease comprises an amino acid sequence with at least 95% identity to SEQ ID NO: 4 and the direct repeat sequence comprises the nucleotide sequence set forth in SEQ ID NO: 71 or SEQ ID NO: 72,
(e) the nuclease comprises an amino acid sequence with at least 95% identity to SEQ ID NO: 5 and the direct repeat sequence comprises the nucleotide sequence set forth in SEQ ID NO: 73 or SEQ ID NO: 74,
(f) the nuclease comprises an amino acid sequence with at least 95% identity to SEQ ID NO: 6 and the direct repeat sequence comprises the nucleotide sequence set forth in SEQ ID NO: 75 or SEQ ID NO: 76,
(g) the nuclease comprises an amino acid sequence with at least 95% identity to SEQ ID NO: 7 and the direct repeat sequence comprises the nucleotide sequence set forth in SEQ ID NO: 77 or SEQ ID NO: 78,
(h) the nuclease comprises an amino acid sequence with at least 95% identity to SEQ ID NO: 8 and the direct repeat sequence comprises the nucleotide sequence set forth in SEQ ID NO: 79 or SEQ ID NO: 80,
(i) the nuclease comprises an amino acid sequence with at least 95% identity to SEQ ID NO: 9 and the direct repeat sequence comprises the nucleotide sequence set forth in SEQ ID NO: 81 or SEQ ID NO: 82,
(j) the nuclease comprises an amino acid sequence with at least 95% identity to SEQ ID NO: 10 and the direct repeat sequence comprises the nucleotide sequence set forth in SEQ ID NO: 83 or SEQ ID NO: 84,
(k) the nuclease comprises an amino acid sequence with at least 95% identity to SEQ ID NO: 11 and the direct repeat sequence comprises the nucleotide sequence set forth in SEQ ID NO: 85 or SEQ ID NO: 86,
(l) the nuclease comprises an amino acid sequence with at least 95% identity to SEQ ID NO: 12 and the direct repeat sequence comprises the nucleotide sequence set forth in SEQ ID NO: 87 or SEQ ID NO: 88,
(m) the nuclease comprises an amino acid sequence with at least 95% identity to SEQ ID NO: 13 and the direct repeat sequence comprises the nucleotide sequence set forth in SEQ ID NO: 89 or SEQ ID NO: 90,
(n) the nuclease comprises an amino acid sequence with at least 95% identity to SEQ ID NO: 14 and the direct repeat sequence comprises the nucleotide sequence set forth in SEQ ID NO: 91 or SEQ ID NO: 92,
(o) the nuclease comprises an amino acid sequence with at least 95% identity to SEQ ID NO: 15 and the direct repeat sequence comprises the nucleotide sequence set forth in SEQ ID NO: 93 or SEQ ID NO: 94,
(p) the nuclease comprises an amino acid sequence with at least 95% identity to SEQ ID NO: 16 and the direct repeat sequence comprises the nucleotide sequence set forth in SEQ ID NO: 95 or SEQ ID NO: 96,
(q) the nuclease comprises an amino acid sequence with at least 95% identity to SEQ ID NO: 17 and the direct repeat sequence comprises the nucleotide sequence set forth in SEQ ID NO: 97 or SEQ ID NO: 98,
(r) the nuclease comprises an amino acid sequence with at least 95% identity to SEQ ID NO: 18 and the direct repeat sequence comprises the nucleotide sequence set forth in SEQ ID NO: 99 or SEQ ID NO: 100,
(s) the nuclease comprises an amino acid sequence with at least 95% identity to SEQ ID NO: 19 and the direct repeat sequence comprises the nucleotide sequence set forth in SEQ ID NO: 101 or SEQ ID NO: 102,
(t) the nuclease comprises an amino acid sequence with at least 95% identity to SEQ ID NO: 20 and the direct repeat sequence comprises the nucleotide sequence set forth in SEQ ID NO: 103 or SEQ ID NO: 104,
(u) the nuclease comprises an amino acid sequence with at least 95% identity to SEQ ID NO: 21 and the direct repeat sequence comprises the nucleotide sequence set forth in SEQ ID NO: 105 or SEQ ID NO: 106,
(v) the nuclease comprises an amino acid sequence with at least 95% identity to SEQ ID NO: 22 and the direct repeat sequence comprises the nucleotide sequence set forth in SEQ ID NO: 107 or SEQ ID NO: 108,
(w) the nuclease comprises an amino acid sequence with at least 95% identity to SEQ ID NO: 23 and the direct repeat sequence comprises the nucleotide sequence set forth in SEQ ID NO: 109 or SEQ ID NO: 110,
(x) the nuclease comprises an amino acid sequence with at least 95% identity to SEQ ID NO: 24 and the direct repeat sequence comprises the nucleotide sequence set forth in SEQ ID NO: 111 or SEQ ID NO: 112,
(y) the nuclease comprises an amino acid sequence with at least 95% identity to SEQ ID NO: 25 and the direct repeat sequence comprises the nucleotide sequence set forth in SEQ ID NO: 113 or SEQ ID NO: 114,
(z) the nuclease comprises an amino acid sequence with at least 95% identity to SEQ ID NO: 26 and the direct repeat sequence comprises the nucleotide sequence set forth in SEQ ID NO: 115 or SEQ ID NO: 116,
(aa) the nuclease comprises an amino acid sequence with at least 95% identity to SEQ ID NO: 27 and the direct repeat sequence comprises the nucleotide sequence set forth in SEQ ID NO: 117 or SEQ ID NO: 118,
(bb) the nuclease comprises an amino acid sequence with at least 95% identity to SEQ ID NO: 28 and the direct repeat sequence comprises the nucleotide sequence set forth in SEQ ID NO: 119 or SEQ ID NO: 120,
(cc) the nuclease comprises an amino acid sequence with at least 95% identity to SEQ ID NO: 29 and the direct repeat sequence comprises the nucleotide sequence set forth in SEQ ID NO: 121 or SEQ ID NO: 122,
(dd) the nuclease comprises an amino acid sequence with at least 95% identity to SEQ ID NO: 30 and the direct repeat sequence comprises the nucleotide sequence set forth in SEQ ID NO: 123 or SEQ ID NO: 124,
(ee) the nuclease comprises an amino acid sequence with at least 95% identity to SEQ ID NO: 31 and the direct repeat sequence comprises the nucleotide sequence set forth in SEQ ID NO: 125 or SEQ ID NO: 126; or
(ff) the nuclease comprises an amino acid sequence with at least 95% identity to SEQ ID NO: 32 and the direct repeat sequence comprises the nucleotide sequence set forth in SEQ ID NO: 127 or SEQ ID NO: 128.

8. The gene editing system of claim 1, wherein the RNA guide further comprises a trans-activating crRNA (tracrRNA) sequence.

9. The gene editing system of claim 8, wherein:
(a) the nuclease comprises an amino acid sequence with at least 95% identity to SEQ ID NO: 1 and the tracrRNA sequence comprises a nucleotide sequence with at least 90% identity to SEQ ID NO: 129,
(b) the nuclease comprises an amino acid sequence with at least 95% identity to SEQ ID NO: 2 and the tracrRNA sequence comprises a nucleotide sequence with at least 90% identity to any one of SEQ ID NOs: 130-137,
(c) the nuclease comprises an amino acid sequence with at least 95% identity to SEQ ID NO: 3 and the tracrRNA sequence comprises a nucleotide sequence with at least 90% identity to any one of SEQ ID NOs: 138-147,
(d) the nuclease comprises an amino acid sequence with at least 95% identity to SEQ ID NO: 4 and the tracrRNA sequence comprises a nucleotide sequence with at least 90% identity to any one of SEQ ID NOs: 148-149,
(e) the nuclease comprises an amino acid sequence with at least 95% identity to SEQ ID NO: 5 and the tracrRNA sequence comprises a nucleotide sequence with at least 90% identity to any one of SEQ ID NOs: 150-163,
(f) the nuclease comprises an amino acid sequence with at least 95% identity to SEQ ID NO: 6 and the tracrRNA sequence comprises a nucleotide sequence with at least 90% identity to any one of SEQ ID NOs: 164-169,
(g) the nuclease comprises an amino acid sequence with at least 95% identity to SEQ ID NO: 7 and the tracrRNA sequence comprises a nucleotide sequence with at least 90% identity to any one of SEQ ID NOs: 170-175,
(h) the nuclease comprises an amino acid sequence with at least 95% identity to SEQ ID NO: 8 and the tracrRNA sequence comprises a nucleotide sequence with at least 90% identity to any one of SEQ ID NOs: 176-177,
(i) the nuclease comprises an amino acid sequence with at least 95% identity to SEQ ID NO: 9 and the tracrRNA sequence comprises a nucleotide sequence with at least 90% identity to any one of SEQ ID NOs: 178-183,
(j) the nuclease comprises an amino acid sequence with at least 95% identity to SEQ ID NO: 10 and the tracrRNA sequence comprises a nucleotide sequence with at least 90% identity to any one of SEQ ID NOs: 184-193,
(k) the nuclease comprises an amino acid sequence with at least 95% identity to SEQ ID NO: 11 and the tracrRNA sequence comprises a nucleotide sequence with at least 90% identity to any one of SEQ ID NOs: 194-199,
(l) the nuclease comprises an amino acid sequence with at least 95% identity to SEQ ID NO: 12 and the tracrRNA sequence comprises a nucleotide sequence with at least 90% identity to any one of SEQ ID NOs: 200-211,
(m) the nuclease comprises an amino acid sequence with at least 95% identity to SEQ ID NO: 13 and the tracrRNA sequence comprises a nucleotide sequence with at least 90% identity to any one of SEQ ID NOs: 212-219,
(n) the nuclease comprises an amino acid sequence with at least 95% identity to SEQ ID NO: 14 and the tracrRNA sequence comprises a nucleotide sequence with at least 90% identity to any one of SEQ ID NOs: 220-227,
(o) the nuclease comprises an amino acid sequence with at least 95% identity to SEQ ID NO: 15 and the tracrRNA sequence comprises a nucleotide sequence with at least 90% identity to any one of SEQ ID NOs: 228-233,
(p) the nuclease comprises an amino acid sequence with at least 95% identity to SEQ ID NO: 16 and the tracrRNA sequence comprises a nucleotide sequence with at least 90% identity to any one of SEQ ID NOs: 234-237,
(q) the nuclease comprises an amino acid sequence with at least 95% identity to SEQ ID NO: 17 and the tracrRNA sequence comprises a nucleotide sequence with at least 90% identity to any one of SEQ ID NOs: 238-243,
(r) the nuclease comprises an amino acid sequence with at least 95% identity to SEQ ID NO: 18 and the tracrRNA sequence comprises a nucleotide sequence with at least 90% identity to any one of SEQ ID NOs: 244-247,
(s) the nuclease comprises an amino acid sequence with at least 95% identity to SEQ ID NO: 19 and the tracrRNA sequence comprises a nucleotide sequence with at least 90% identity to SEQ ID NO: 248,
(t) the nuclease comprises an amino acid sequence with at least 95% identity to SEQ ID NO: 20 and the tracrRNA sequence comprises a nucleotide sequence with at least 90% identity to any one of SEQ ID NOs: 249-254,
(u) the nuclease comprises an amino acid sequence with at least 95% identity to SEQ ID NO: 21 and the tracrRNA sequence comprises a nucleotide sequence with at least 90% identity to any one of SEQ ID NOs: 255-256,
(v) the nuclease comprises an amino acid sequence with at least 95% identity to SEQ ID NO: 22 and the tracrRNA sequence comprises a nucleotide sequence with at least 90% identity to any one of SEQ ID NOs: 257-260,
(w) the nuclease comprises an amino acid sequence with at least 95% identity to SEQ ID NO: 23 and the tracrRNA sequence comprises a nucleotide sequence with at least 90% identity to any one of SEQ ID NOs: 261-264,
(x) the nuclease comprises an amino acid sequence with at least 95% identity to SEQ ID NO: 24 and the tracrRNA sequence comprises a nucleotide sequence with at least 90% identity to any one of SEQ ID NOs: 265-268,
(y) the nuclease comprises an amino acid sequence with at least 95% identity to SEQ ID NO: 25 and the tracrRNA sequence comprises a nucleotide sequence with at least 90% identity to any one of SEQ ID NOs: 269-272,
(z) the nuclease comprises an amino acid sequence with at least 95% identity to SEQ ID NO: 26 and the tracrRNA sequence comprises a nucleotide sequence with at least 90% identity to SEQ ID NO: 273,
(aa) the nuclease comprises an amino acid sequence with at least 95% identity to SEQ ID NO: 27 and the tracrRNA sequence comprises a nucleotide sequence with at least 90% identity to SEQ ID NO: 274,
(bb) the nuclease comprises an amino acid sequence with at least 95% identity to SEQ ID NO: 28 and the tracrRNA sequence comprises a nucleotide sequence with at least 90% identity to SEQ ID NO: 275,
(cc) the nuclease comprises an amino acid sequence with at least 95% identity to SEQ ID NO: 29 and the tracrRNA sequence comprises a nucleotide sequence with at least 90% identity to any one of SEQ ID NOs: 276-281,
(dd) the nuclease comprises an amino acid sequence with at least 95% identity to SEQ ID NO: 30 and the tracrRNA sequence comprises a nucleotide sequence with at least 90% identity to any one of SEQ ID NOs: 282-289,
(ee) the nuclease comprises an amino acid sequence with at least 95% identity to SEQ ID NO: 31 and the tracrRNA sequence comprises a nucleotide sequence with at least 90% identity to any one of SEQ ID NOs: 290-293; or
(ff) the nuclease comprises an amino acid sequence with at least 95% identity to SEQ ID NO: 32 and the tracrRNA sequence comprises a nucleotide sequence with at least 90% identity to any one of SEQ ID NOs: 294-299.

10. The gene editing system of claim 8, wherein:
(a) the nuclease comprises an amino acid sequence with at least 95% identity to SEQ ID NO: 1 and the tracrRNA sequence comprises a nucleotide sequence with at least 95% identity to SEQ ID NO: 129,
(b) the nuclease comprises an amino acid sequence with at least 95% identity to SEQ ID NO: 2 and the tracrRNA sequence comprises a nucleotide sequence with at least 95% identity to any one of SEQ ID NOs: 130-137,
(c) the nuclease comprises an amino acid sequence with at least 95% identity to SEQ ID NO: 3 and the tracrRNA sequence comprises a nucleotide sequence with at least 95% identity to any one of SEQ ID NOs: 138-147,
(d) the nuclease comprises an amino acid sequence with at least 95% identity to SEQ ID NO: 4 and the tracrRNA sequence comprises a nucleotide sequence with at least 95% identity to any one of SEQ ID NOs: 148-149,
(e) the nuclease comprises an amino acid sequence with at least 95% identity to SEQ ID NO: 5 and the tracrRNA sequence comprises a nucleotide sequence with at least 95% identity to any one of SEQ ID NOs: 150-163,
(f) the nuclease comprises an amino acid sequence with at least 95% identity to SEQ ID NO: 6 and the tracrRNA sequence comprises a nucleotide sequence with at least 95% identity to any one of SEQ ID NOs: 164-169,
(g) the nuclease comprises an amino acid sequence with at least 95% identity to SEQ ID NO: 7 and the tracrRNA sequence comprises a nucleotide sequence with at least 95% identity to any one of SEQ ID NOs: 170-175,
(h) the nuclease comprises an amino acid sequence with at least 95% identity to SEQ ID NO: 8 and the tracrRNA sequence comprises a nucleotide sequence with at least 95% identity to any one of SEQ ID NOs: 176-177,
(i) the nuclease comprises an amino acid sequence with at least 95% identity to SEQ ID NO: 9 and the tracrRNA sequence comprises a nucleotide sequence with at least 95% identity to any one of SEQ ID NOs: 178-183,
(j) the nuclease comprises an amino acid sequence with at least 95% identity to SEQ ID NO: 10 and the tracrRNA sequence comprises a nucleotide sequence with at least 95% identity to any one of SEQ ID NOs: 184-193,
(k) the nuclease comprises an amino acid sequence with at least 95% identity to SEQ ID NO: 11 and the tracrRNA sequence comprises a nucleotide sequence with at least 95% identity to any one of SEQ ID NOs: 194-199,
(l) the nuclease comprises an amino acid sequence with at least 95% identity to SEQ ID NO: 12 and the tracrRNA sequence comprises a nucleotide sequence with at least 95% identity to any one of SEQ ID NOs: 200-211,
(m) the nuclease comprises an amino acid sequence with at least 95% identity to SEQ ID NO: 13 and the tracrRNA sequence comprises a nucleotide sequence with at least 95% identity to any one of SEQ ID NOs: 212-219,
(n) the nuclease comprises an amino acid sequence with at least 95% identity to SEQ ID NO: 14 and the tracrRNA sequence comprises a nucleotide sequence with at least 95% identity to any one of SEQ ID NOs: 220-227,
(o) the nuclease comprises an amino acid sequence with at least 95% identity to SEQ ID NO: 15 and the tracrRNA sequence comprises a nucleotide sequence with at least 95% identity to any one of SEQ ID NOs: 228-233,
(p) the nuclease comprises an amino acid sequence with at least 95% identity to SEQ ID NO: 16 and the tracrRNA sequence comprises a nucleotide sequence with at least 95% identity to any one of SEQ ID NOs: 234-237,
(q) the nuclease comprises an amino acid sequence with at least 95% identity to SEQ ID NO: 17 and the tracrRNA sequence comprises a nucleotide sequence with at least 95% identity to any one of SEQ ID NOs: 238-243,
(r) the nuclease comprises an amino acid sequence with at least 95% identity to SEQ ID NO: 18 and the tracrRNA sequence comprises a nucleotide sequence with at least 95% identity to any one of SEQ ID NOs: 244-247,
(s) the nuclease comprises an amino acid sequence with at least 95% identity to SEQ ID NO: 19 and the tracrRNA sequence comprises a nucleotide sequence with at least 95% identity to SEQ ID NO: 248,
(t) the nuclease comprises an amino acid sequence with at least 95% identity to SEQ ID NO: 20 and the tracrRNA sequence comprises a nucleotide sequence with at least 95% identity to any one of SEQ ID NOs: 249-254,
(u) the nuclease comprises an amino acid sequence with at least 95% identity to SEQ ID NO: 21 and the tracrRNA sequence comprises a nucleotide sequence with at least 95% identity to any one of SEQ ID NOs: 255-256,
(v) the nuclease comprises an amino acid sequence with at least 95% identity to SEQ ID NO: 22 and the tracrRNA sequence comprises a nucleotide sequence with at least 95% identity to any one of SEQ ID NOs: 257-260,
(w) the nuclease comprises an amino acid sequence with at least 95% identity to SEQ ID NO: 23 and the tracrRNA sequence comprises a nucleotide sequence with at least 95% identity to any one of SEQ ID NOs: 261-264,
(x) the nuclease comprises an amino acid sequence with at least 95% identity to SEQ ID NO: 24 and the tracrRNA sequence comprises a nucleotide sequence with at least 95% identity to any one of SEQ ID NOs: 265-268,
(y) the nuclease comprises an amino acid sequence with at least 95% identity to SEQ ID NO: 25 and the tracrRNA sequence comprises a nucleotide sequence with at least 95% identity to any one of SEQ ID NOs: 269-272,
(z) the nuclease comprises an amino acid sequence with at least 95% identity to SEQ ID NO: 26 and the tracrRNA sequence comprises a nucleotide sequence with at least 95% identity to SEQ ID NO: 273,
(aa) the nuclease comprises an amino acid sequence with at least 95% identity to SEQ ID NO: 27 and the tracrRNA sequence comprises a nucleotide sequence with at least 95% identity to SEQ ID NO: 274,
(bb) the nuclease comprises an amino acid sequence with at least 95% identity to SEQ ID NO: 28 and the tracrRNA sequence comprises a nucleotide sequence with at least 95% identity to SEQ ID NO: 275,
(cc) the nuclease comprises an amino acid sequence with at least 95% identity to SEQ ID NO: 29 and the tracrRNA sequence comprises a nucleotide sequence with at least 95% identity to any one of SEQ ID NOs: 276-281,
(dd) the nuclease comprises an amino acid sequence with at least 95% identity to SEQ ID NO: 30 and the tracrRNA sequence comprises a nucleotide sequence with at least 95% identity to any one of SEQ ID NOs: 282-289,
(ee) the nuclease comprises an amino acid sequence with at least 95% identity to SEQ ID NO: 31 and the tracrRNA sequence comprises a nucleotide sequence with at least 95% identity to any one of SEQ ID NOs: 290-293; or
(ff) the nuclease comprises an amino acid sequence with at least 95% identity to SEQ ID NO: 32 and the tracrRNA sequence comprises a nucleotide sequence with at least 95% identity to any one of SEQ ID NOs: 294-299.

11. The gene editing system of claim 8, wherein:
(a) the nuclease comprises an amino acid sequence with at least 95% identity to SEQ ID NO: 1 and the tracrRNA sequence comprises a nucleotide sequence set forth in SEQ ID NO: 129,
(b) the nuclease comprises an amino acid sequence with at least 95% identity to SEQ ID NO: 2 and the tracrRNA sequence comprises the nucleotide sequence set forth in any one of SEQ ID NOs: 130-137,
(c) the nuclease comprises an amino acid sequence with at least 95% identity to SEQ ID NO: 3 and the tracrRNA sequence comprises the nucleotide sequence set forth in any one of SEQ ID NOs: 138-147,
(d) the nuclease comprises an amino acid sequence with at least 95% identity to SEQ ID NO: 4 and the tracrRNA sequence comprises the nucleotide sequence set forth in any one of SEQ ID NOs: 148-149,
(e) the nuclease comprises an amino acid sequence with at least 95% identity to SEQ ID NO: 5 and the tracrRNA sequence comprises the nucleotide sequence set forth in any one of SEQ ID NOs: 150-163,
(f) the nuclease comprises an amino acid sequence with at least 95% identity to SEQ ID NO: 6 and the tracrRNA sequence comprises the nucleotide sequence set forth in any one of SEQ ID NOs: 164-169,
(g) the nuclease comprises an amino acid sequence with at least 95% identity to SEQ ID NO: 7 and the tracrRNA sequence comprises the nucleotide sequence set forth in any one of SEQ ID NOs: 170-175,
(h) the nuclease comprises an amino acid sequence with at least 95% identity to SEQ ID NO: 8 and the tracrRNA sequence comprises the nucleotide sequence set forth in any one of SEQ ID NOs: 176-177,
(i) the nuclease comprises an amino acid sequence with at least 95% identity to SEQ ID NO: 9 and the tracrRNA sequence comprises the nucleotide sequence set forth in any one of SEQ ID NOs: 178-183,
(j) the nuclease comprises an amino acid sequence with at least 95% identity to SEQ ID NO: 10 and the tracrRNA sequence comprises the nucleotide sequence set forth in any one of SEQ ID NOs: 184-193,
(k) the nuclease comprises an amino acid sequence with at least 95% identity to SEQ ID NO: 11 and the tracrRNA sequence comprises the nucleotide sequence set forth in any one of SEQ ID NOs: 194-199,
(l) the nuclease comprises an amino acid sequence with at least 95% identity to SEQ ID NO: 12 and the tracrRNA sequence comprises the nucleotide sequence set forth in any one of SEQ ID NOs: 200-211,
(m) the nuclease comprises an amino acid sequence with at least 95% identity to SEQ ID NO: 13 and the tracrRNA sequence comprises the nucleotide sequence set forth in any one of SEQ ID NOs: 212-219,
(n) the nuclease comprises an amino acid sequence with at least 95% identity to SEQ ID NO: 14 and the tracrRNA sequence comprises the nucleotide sequence set forth in any one of SEQ ID NOs: 220-227,
(o) the nuclease comprises an amino acid sequence with at least 95% identity to SEQ ID NO: 15 and the tracrRNA sequence comprises the nucleotide sequence set forth in any one of SEQ ID NOs: 228-233,
(p) the nuclease comprises an amino acid sequence with at least 95% identity to SEQ ID NO: 16 and the tracrRNA sequence comprises the nucleotide sequence set forth in any one of SEQ ID NOs: 234-237,
(q) the nuclease comprises an amino acid sequence with at least 95% identity to SEQ ID NO: 17 and the tracrRNA sequence comprises the nucleotide sequence set forth in any one of SEQ ID NOs: 238-243,
(r) the nuclease comprises an amino acid sequence with at least 95% identity to SEQ ID NO: 18 and the tracrRNA sequence comprises the nucleotide sequence set forth in any one of SEQ ID NOs: 244-247,
(s) the nuclease comprises an amino acid sequence with at least 95% identity to SEQ ID NO: 19 and the tracrRNA sequence comprises the nucleotide sequence set forth in SEQ ID NO: 248,
(t) the nuclease comprises an amino acid sequence with at least 95% identity to SEQ ID NO: 20 and the tracrRNA sequence comprises the nucleotide sequence set forth in any one of SEQ ID NOs: 249-254,
(u) the nuclease comprises an amino acid sequence with at least 95% identity to SEQ ID NO: 21 and the tracrRNA sequence comprises the nucleotide sequence set forth in any one of SEQ ID NOs: 255-256,
(v) the nuclease comprises an amino acid sequence with at least 95% identity to SEQ ID NO: 22 and the tracrRNA sequence comprises the nucleotide sequence set forth in any one of SEQ ID NOs: 257-260,
(w) the nuclease comprises an amino acid sequence with at least 95% identity to SEQ ID NO: 23 and the tracrRNA sequence comprises the nucleotide sequence set forth in any one of SEQ ID NOs: 261-264,
(x) the nuclease comprises an amino acid sequence with at least 95% identity to SEQ ID NO: 24 and the tracrRNA sequence comprises the nucleotide sequence set forth in any one of SEQ ID NOs: 265-268,
(y) the nuclease comprises an amino acid sequence with at least 95% identity to SEQ ID NO: 25 and the tracrRNA sequence comprises the nucleotide sequence set forth in any one of SEQ ID NOs: 269-272,
(z) the nuclease comprises an amino acid sequence with at least 95% identity to SEQ ID NO: 26 and the tracrRNA sequence comprises the nucleotide sequence set forth in SEQ ID NO: 273,
(aa) the nuclease comprises an amino acid sequence with at least 95% identity to SEQ ID NO: 27 and the tracrRNA sequence comprises the nucleotide sequence set forth in SEQ ID NO: 274,
(bb) the nuclease comprises an amino acid sequence with at least 95% identity to SEQ ID NO: 28 and the tracrRNA sequence comprises the nucleotide sequence set forth in SEQ ID NO: 275,
(cc) the nuclease comprises an amino acid sequence with at least 95% identity to SEQ ID NO: 29 and the tracrRNA sequence comprises the nucleotide sequence set forth in any one of SEQ ID NOs: 276-281,
(dd) the nuclease comprises an amino acid sequence with at least 95% identity to SEQ ID NO: 30 and the tracrRNA sequence comprises the nucleotide sequence set forth in any one of SEQ ID NOs: 282-289,
(ee) the nuclease comprises an amino acid sequence with at least 95% identity to SEQ ID NO: 31 and the tracrRNA sequence comprises the nucleotide sequence set forth in any one of SEQ ID NOs: 290-293; or
(ff) the nuclease comprises an amino acid sequence with at least 95% identity to SEQ ID NO: 32 and the tracrRNA sequence comprises the nucleotide sequence set forth in any one of SEQ ID NOs: 294-299.

12. The gene editing system of claim 8, wherein the tracrRNA sequence is fused to the direct repeat sequence.

13. The gene editing system of claim 1, wherein the RNA guide is a single molecule RNA guide (sgRNA).

14. The gene editing system of claim 13, wherein:
(a) the nuclease comprises an amino acid sequence with at least 95% identity to SEQ ID NO: 1 and the sgRNA comprises a nucleotide sequence with at least 90% identity to SEQ ID NO: 300,
(b) the nuclease comprises an amino acid sequence with at least 95% identity to SEQ ID NO: 2 and the sgRNA comprises a nucleotide sequence with at least 90% identity to any one of SEQ ID NOs: 301-308,
(c) the nuclease comprises an amino acid sequence with at least 95% identity to SEQ ID NO: 3 and the sgRNA comprises a nucleotide sequence with at least 90% identity to any one of SEQ ID NOs: 309-318,
(d) the nuclease comprises an amino acid sequence with at least 95% identity to SEQ ID NO: 4 and the sgRNA comprises a nucleotide sequence with at least 90% identity to any one of SEQ ID NOs: 319-320,
(e) the nuclease comprises an amino acid sequence with at least 95% identity to SEQ ID NO: 5 and the sgRNA comprises a nucleotide sequence with at least 90% identity to any one of SEQ ID NOs: 321-334,
(f) the nuclease comprises an amino acid sequence with at least 95% identity to SEQ ID NO: 6 and the sgRNA comprises a nucleotide sequence with at least 90% identity to any one of SEQ ID NOs: 335-340,
(g) the nuclease comprises an amino acid sequence with at least 95% identity to SEQ ID NO: 7 and the sgRNA comprises a nucleotide sequence with at least 90% identity to any one of SEQ ID NOs: 341-346,
(h) the nuclease comprises an amino acid sequence with at least 95% identity to SEQ ID NO: 8 and the sgRNA comprises a nucleotide sequence with at least 90% identity to any one of SEQ ID NOs: 347-348,
(i) the nuclease comprises an amino acid sequence with at least 95% identity to SEQ ID NO: 9 and the sgRNA comprises a nucleotide sequence with at least 90% identity to any one of SEQ ID NOs: 349-354,
(j) the nuclease comprises an amino acid sequence with at least 95% identity to SEQ ID NO: 10 and the sgRNA comprises a nucleotide sequence with at least 90% identity to any one of SEQ ID NOs: 355-364,
(k) the nuclease comprises an amino acid sequence with at least 95% identity to SEQ ID NO: 11 and the sgRNA comprises a nucleotide sequence with at least 90% identity to any one of SEQ ID NOs: 365-370,
(l) the nuclease comprises an amino acid sequence with at least 95% identity to SEQ ID NO: 12 and the sgRNA comprises a nucleotide sequence with at least 90% identity to any one of SEQ ID NOs: 371-382,
(m) the nuclease comprises an amino acid sequence with at least 95% identity to SEQ ID NO: 13 and the sgRNA comprises a nucleotide sequence with at least 90% identity to any one of SEQ ID NOs: 383-390,
(n) the nuclease comprises an amino acid sequence with at least 95% identity to SEQ ID NO: 14 and the sgRNA comprises a nucleotide sequence with at least 90% identity to any one of SEQ ID NOs: 391-398,
(o) the nuclease comprises an amino acid sequence with at least 95% identity to SEQ ID NO: 15 and the sgRNA comprises a nucleotide sequence with at least 90% identity to any one of SEQ ID NOs: 399-404,
(p) the nuclease comprises an amino acid sequence with at least 95% identity to SEQ ID NO: 16 and the sgRNA comprises a nucleotide sequence with at least 90% identity to any one of SEQ ID NOs: 405-408,
(q) the nuclease comprises an amino acid sequence with at least 95% identity to SEQ ID NO: 17 and the sgRNA comprises a nucleotide sequence with at least 90% identity to any one of SEQ ID NOs: 409-414,
(r) the nuclease comprises an amino acid sequence with at least 95% identity to SEQ ID NO: 18 and the sgRNA comprises a nucleotide sequence with at least 90% identity to any one of SEQ ID NOs: 415-418,
(s) the nuclease comprises an amino acid sequence with at least 95% identity to SEQ ID NO: 19 and the sgRNA comprises a nucleotide sequence with at least 90% identity to SEQ ID NO: 419,
(t) the nuclease comprises an amino acid sequence with at least 95% identity to SEQ ID NO: 20 and the sgRNA comprises a nucleotide sequence with at least 90% identity to any one of SEQ ID NOs: 420-425,
(u) the nuclease comprises an amino acid sequence with at least 95% identity to SEQ ID NO: 21 and the sgRNA comprises a nucleotide sequence with at least 90% identity to any one of SEQ ID NOs: 426-427,
(v) the nuclease comprises an amino acid sequence with at least 95% identity to SEQ ID NO: 22 and the sgRNA comprises a nucleotide sequence with at least 90% identity to any one of SEQ ID NOs: 428-431,
(w) the nuclease comprises an amino acid sequence with at least 95% identity to SEQ ID NO: 23 and the sgRNA comprises a nucleotide sequence with at least 90% identity to any one of SEQ ID NOs: 432-435,
(x) the nuclease comprises an amino acid sequence with at least 95% identity to SEQ ID NO: 24 and the sgRNA comprises a nucleotide sequence with at least 90% identity to any one of SEQ ID NOs: 436-439,
(y) the nuclease comprises an amino acid sequence with at least 95% identity to SEQ ID NO: 25 and the sgRNA comprises a nucleotide sequence with at least 90% identity to any one of SEQ ID NOs: 440-443,
(z) the nuclease comprises an amino acid sequence with at least 95% identity to SEQ ID NO: 26 and the sgRNA comprises a nucleotide sequence with at least 90% identity to SEQ ID NO: 444,
(aa) the nuclease comprises an amino acid sequence with at least 95% identity to SEQ ID NO: 27 and the sgRNA comprises a nucleotide sequence with at least 90% identity to SEQ ID NO: 445,
(bb) the nuclease comprises an amino acid sequence with at least 95% identity to SEQ ID NO: 28 and the sgRNA comprises a nucleotide sequence with at least 90% identity to SEQ ID NO: 446,
(cc) the nuclease comprises an amino acid sequence with at least 95% identity to SEQ ID NO: 29 and the sgRNA comprises a nucleotide sequence with at least 90% identity to any one of SEQ ID NOs: 447-452,
(dd) the nuclease comprises an amino acid sequence with at least 95% identity to SEQ ID NO: 30 and the sgRNA comprises a nucleotide sequence with at least 90% identity to any one of SEQ ID NOs: 453-460,
(ee) the nuclease comprises an amino acid sequence with at least 95% identity to SEQ ID NO: 31 and the sgRNA comprises a nucleotide sequence with at least 90% identity to any one of SEQ ID NOs: 461-464; or
(ff) the nuclease comprises an amino acid sequence with at least 95% identity to SEQ ID NO: 32 and the sgRNA comprises a nucleotide sequence with at least 90% identity to any one of SEQ ID NOs: 465-469.

15. The gene editing system of claim 13, wherein:
(a) the nuclease comprises an amino acid sequence with at least 95% identity to SEQ ID NO: 1 and the sgRNA comprises a nucleotide sequence with at least 95% identity to SEQ ID NO: 300,
(b) the nuclease comprises an amino acid sequence with at least 95% identity to SEQ ID NO: 2 and the sgRNA comprises a nucleotide sequence with at least 95% identity to any one of SEQ ID NOs: 301-308,
(c) the nuclease comprises an amino acid sequence with at least 95% identity to SEQ ID NO: 3 and the sgRNA comprises a nucleotide sequence with at least 95% identity to any one of SEQ ID NOs: 309-318,
(d) the nuclease comprises an amino acid sequence with at least 95% identity to SEQ ID NO: 4 and the sgRNA comprises a nucleotide sequence with at least 95% identity to any one of SEQ ID NOs: 319-320,
(e) the nuclease comprises an amino acid sequence with at least 95% identity to SEQ ID NO: 5 and the sgRNA comprises a nucleotide sequence with at least 95% identity to any one of SEQ ID NOs: 321-334,
(f) the nuclease comprises an amino acid sequence with at least 95% identity to SEQ ID NO: 6 and the sgRNA comprises a nucleotide sequence with at least 95% identity to any one of SEQ ID NOs: 335-340,
(g) the nuclease comprises an amino acid sequence with at least 95% identity to SEQ ID NO: 7 and the sgRNA comprises a nucleotide sequence with at least 95% identity to any one of SEQ ID NOs: 341-346,
(h) the nuclease comprises an amino acid sequence with at least 95% identity to SEQ ID NO: 8 and the sgRNA comprises a nucleotide sequence with at least 95% identity to any one of SEQ ID NOs: 347-348,
(i) the nuclease comprises an amino acid sequence with at least 95% identity to SEQ ID NO: 9 and the sgRNA comprises a nucleotide sequence with at least 95% identity to any one of SEQ ID NOs: 349-354,
(j) the nuclease comprises an amino acid sequence with at least 95% identity to SEQ ID NO: 10 and the sgRNA comprises a nucleotide sequence with at least 95% identity to any one of SEQ ID NOs: 355-364,
(k) the nuclease comprises an amino acid sequence with at least 95% identity to SEQ ID NO: 11 and the sgRNA comprises a nucleotide sequence with at least 95% identity to any one of SEQ ID NOs: 365-370,
(l) the nuclease comprises an amino acid sequence with at least 95% identity to SEQ ID NO: 12 and the sgRNA comprises a nucleotide sequence with at least 95% identity to any one of SEQ ID NOs: 371-382,
(m) the nuclease comprises an amino acid sequence with at least 95% identity to SEQ ID NO: 13 and the sgRNA comprises a nucleotide sequence with at least 95% identity to any one of SEQ ID NOs: 383-390,
(n) the nuclease comprises an amino acid sequence with at least 95% identity to SEQ ID NO: 14 and the sgRNA comprises a nucleotide sequence with at least 95% identity to any one of SEQ ID NOs: 391-398,
(o) the nuclease comprises an amino acid sequence with at least 95% identity to SEQ ID NO: 15 and the sgRNA comprises a nucleotide sequence with at least 95% identity to any one of SEQ ID NOs: 399-404,
(p) the nuclease comprises an amino acid sequence with at least 95% identity to SEQ ID NO: 16 and the sgRNA comprises a nucleotide sequence with at least 95% identity to any one of SEQ ID NOs: 405-408,
(q) the nuclease comprises an amino acid sequence with at least 95% identity to SEQ ID NO: 17 and the sgRNA comprises a nucleotide sequence with at least 95% identity to any one of SEQ ID NOs: 409-414,
(r) the nuclease comprises an amino acid sequence with at least 95% identity to SEQ ID NO: 18 and the sgRNA comprises a nucleotide sequence with at least 95% identity to any one of SEQ ID NOs: 415-418,
(s) the nuclease comprises an amino acid sequence with at least 95% identity to SEQ ID NO: 19 and the sgRNA comprises a nucleotide sequence with at least 95% identity to SEQ ID NO: 419,
(t) the nuclease comprises an amino acid sequence with at least 95% identity to SEQ ID NO: 20 and the sgRNA comprises a nucleotide sequence with at least 95% identity to any one of SEQ ID NOs: 420-425,
(u) the nuclease comprises an amino acid sequence with at least 95% identity to SEQ ID NO: 21 and the sgRNA comprises a nucleotide sequence with at least 95% identity to any one of SEQ ID NOs: 426-427,
(v) the nuclease comprises an amino acid sequence with at least 95% identity to SEQ ID NO: 22 and the sgRNA comprises a nucleotide sequence with at least 95% identity to any one of SEQ ID NOs: 428-431,
(w) the nuclease comprises an amino acid sequence with at least 95% identity to SEQ ID NO: 23 and the sgRNA comprises a nucleotide sequence with at least 95% identity to any one of SEQ ID NOs: 432-435,
(x) the nuclease comprises an amino acid sequence with at least 95% identity to SEQ ID NO: 24 and the sgRNA comprises a nucleotide sequence with at least 95% identity to any one of SEQ ID NOs: 436-439,
(y) the nuclease comprises an amino acid sequence with at least 95% identity to SEQ ID NO: 25 and the sgRNA comprises a nucleotide sequence with at least 95% identity to any one of SEQ ID NOs: 440-443,
(z) the nuclease comprises an amino acid sequence with at least 95% identity to SEQ ID NO: 26 and the sgRNA comprises a nucleotide sequence with at least 95% identity to SEQ ID NO: 444,
(aa) the nuclease comprises an amino acid sequence with at least 95% identity to SEQ ID NO: 27 and the sgRNA comprises a nucleotide sequence with at least 95% identity to SEQ ID NO: 445,
(bb) the nuclease comprises an amino acid sequence with at least 95% identity to SEQ ID NO: 28 and the sgRNA comprises a nucleotide sequence with at least 95% identity to SEQ ID NO: 446,
(cc) the nuclease comprises an amino acid sequence with at least 95% identity to SEQ ID NO: 29 and the sgRNA comprises a nucleotide sequence with at least 95% identity to any one of SEQ ID NOs: 447-452,
(dd) the nuclease comprises an amino acid sequence with at least 95% identity to SEQ ID NO: 30 and the sgRNA comprises a nucleotide sequence with at least 95% identity to any one of SEQ ID NOs: 453-460,
(ee) the nuclease comprises an amino acid sequence with at least 95% identity to SEQ ID NO: 31 and the sgRNA comprises a nucleotide sequence with at least 95% identity to any one of SEQ ID NOs: 461-464; or
(ff) the nuclease comprises an amino acid sequence with at least 95% identity to SEQ ID NO: 32 and the sgRNA comprises a nucleotide sequence with at least 95% identity to any one of SEQ ID NOs: 465-469.

16. The gene editing system of claim 13, wherein:
(a) the nuclease comprises an amino acid sequence with at least 95% identity to SEQ ID NO: 1 and the sgRNA comprises the nucleotide sequence set forth in SEQ ID NO: 300,
(b) the nuclease comprises an amino acid sequence with at least 95% identity to SEQ ID NO: 2 and the sgRNA comprises the nucleotide sequence set forth in any one of SEQ ID NOs: 301-308,
(c) the nuclease comprises an amino acid sequence with at least 95% identity to SEQ ID NO: 3 and the sgRNA comprises the nucleotide sequence set forth in any one of SEQ ID NOs: 309-318,
(d) the nuclease comprises an amino acid sequence with at least 95% identity to SEQ ID NO: 4 and the sgRNA comprises the nucleotide sequence set forth in any one of SEQ ID NOs: 319-320,
(e) the nuclease comprises an amino acid sequence with at least 95% identity to SEQ ID NO: 5 and the sgRNA comprises the nucleotide sequence set forth in any one of SEQ ID NOs: 321-334,
(f) the nuclease comprises an amino acid sequence with at least 95% identity to SEQ ID NO: 6 and the sgRNA comprises the nucleotide sequence set forth in any one of SEQ ID NOs: 335-340,
(g) the nuclease comprises an amino acid sequence with at least 95% identity to SEQ ID NO: 7 and the sgRNA comprises the nucleotide sequence set forth in any one of SEQ ID NOs: 341-346,
(h) the nuclease comprises an amino acid sequence with at least 95% identity to SEQ ID NO: 8 and the sgRNA comprises the nucleotide sequence set forth in any one of SEQ ID NOs: 347-348,
(i) the nuclease comprises an amino acid sequence with at least 95% identity to SEQ ID NO: 9 and the sgRNA comprises the nucleotide sequence set forth in any one of SEQ ID NOs: 349-354,
(j) the nuclease comprises an amino acid sequence with at least 95% identity to SEQ ID NO: 10 and the sgRNA comprises the nucleotide sequence set forth in any one of SEQ ID NOs: 355-364,
(k) the nuclease comprises an amino acid sequence with at least 95% identity to SEQ ID NO: 11 and the sgRNA comprises the nucleotide sequence set forth in any one of SEQ ID NOs: 365-370,
(l) the nuclease comprises an amino acid sequence with at least 95% identity to SEQ ID NO: 12 and the sgRNA comprises the nucleotide sequence set forth in any one of SEQ ID NOs: 371-382,
(m) the nuclease comprises an amino acid sequence with at least 95% identity to SEQ ID NO: 13 and the sgRNA comprises the nucleotide sequence set forth in any one of SEQ ID NOs: 383-390,
(n) the nuclease comprises an amino acid sequence with at least 95% identity to SEQ ID NO: 14 and the sgRNA comprises the nucleotide sequence set forth in any one of SEQ ID NOs: 391-398,
(o) the nuclease comprises an amino acid sequence with at least 95% identity to SEQ ID NO: 15 and the sgRNA comprises the nucleotide sequence set forth in any one of SEQ ID NOs: 399-404,
(p) the nuclease comprises an amino acid sequence with at least 95% identity to SEQ ID NO: 16 and the sgRNA comprises the nucleotide sequence set forth in any one of SEQ ID NOs: 405-408,
(q) the nuclease comprises an amino acid sequence with at least 95% identity to SEQ ID NO: 17 and the sgRNA comprises the nucleotide sequence set forth in any one of SEQ ID NOs: 409-414,
(r) the nuclease comprises an amino acid sequence with at least 95% identity to SEQ ID NO: 18 and the sgRNA comprises the nucleotide sequence set forth in any one of SEQ ID NOs: 415-418,
(s) the nuclease comprises an amino acid sequence with at least 95% identity to SEQ ID NO: 19 and the sgRNA comprises the nucleotide sequence set forth in SEQ ID NO: 419,
(t) the nuclease comprises an amino acid sequence with at least 95% identity to SEQ ID NO: 20 and the sgRNA comprises the nucleotide sequence set forth in any one of SEQ ID NOs: 420-425,
(u) the nuclease comprises an amino acid sequence with at least 95% identity to SEQ ID NO: 21 and the sgRNA comprises the nucleotide sequence set forth in any one of SEQ ID NOs: 426-427,
(v) the nuclease comprises an amino acid sequence with at least 95% identity to SEQ ID NO: 22 and the sgRNA comprises the nucleotide sequence set forth in any one of SEQ ID NOs: 428-431,
(w) the nuclease comprises an amino acid sequence with at least 95% identity to SEQ ID NO: 23 and the sgRNA comprises the nucleotide sequence set forth in any one of SEQ ID NOs: 432-435,
(x) the nuclease comprises an amino acid sequence with at least 95% identity to SEQ ID NO: 24 and the sgRNA comprises the nucleotide sequence set forth in any one of SEQ ID NOs: 436-439,
(y) the nuclease comprises an amino acid sequence with at least 95% identity to SEQ ID NO: 25 and the sgRNA comprises the nucleotide sequence set forth in any one of SEQ ID NOs: 440-443,
(z) the nuclease comprises an amino acid sequence with at least 95% identity to SEQ ID NO: 26 and the sgRNA comprises the nucleotide sequence set forth in SEQ ID NO: 444,
(aa) the nuclease comprises an amino acid sequence with at least 95% identity to SEQ ID NO: 27 and the sgRNA comprises the nucleotide sequence set forth in SEQ ID NO: 445,
(bb) the nuclease comprises an amino acid sequence with at least 95% identity to SEQ ID NO: 28 and the sgRNA comprises the nucleotide sequence set forth in SEQ ID NO: 446,
(cc) the nuclease comprises an amino acid sequence with at least 95% identity to SEQ ID NO: 29 and the sgRNA comprises the nucleotide sequence set forth in any one of SEQ ID NOs: 447-452,
(dd) the nuclease comprises an amino acid sequence with at least 95% identity to SEQ ID NO: 30 and the sgRNA comprises the nucleotide sequence set forth in any one of SEQ ID NOs: 453-460,
(ee) the nuclease comprises an amino acid sequence with at least 95% identity to SEQ ID NO: 31 and the sgRNA comprises the nucleotide sequence set forth in any one of SEQ ID NOs: 461-464; or
(ff) the nuclease comprises an amino acid sequence with at least 95% identity to SEQ ID NO: 32 and the sgRNA comprises the nucleotide sequence set forth in any one of SEQ ID NOs: 465-469.

17. The gene editing system of claim 1, wherein the spacer sequence comprises between about 15 nucleotides and about 50 nucleotides in length.

18. The gene editing system of claim 1, wherein the spacer sequence comprises between about 10 nucleotides and about 35 nucleotides in length.

19. The gene editing system of claim 1, wherein the spacer sequence comprises between about 20 nucleotides and about 25 nucleotides in length.

20. The gene editing system of claim 1, wherein the target sequence is adjacent to a protospacer adjacent motif (PAM) sequence.

21. The gene editing system of claim 20, wherein:
(a) the nuclease comprises an amino acid sequence with at least 95% identity to SEQ ID NO: 1 and the PAM sequence comprises 5′-CC-3′ or 5′-NCC-3′,
(b) the nuclease comprises an amino acid sequence with at least 95% identity to SEQ ID NO: 2 and the PAM sequence comprises 5′-TTC-3′ or 5′-NTTC-3′,
(c) the nuclease comprises an amino acid sequence with at least 95% identity to SEQ ID NO: 3 and the PAM sequence comprises 5′-TY-3′ or 5′-NTY-3′,
(d) the nuclease comprises an amino acid sequence with at least 95% identity to SEQ ID NO: 4 and the PAM sequence comprises 5′-ATC-3′ or 5′-NATC-3′,
(e) the nuclease comprises an amino acid sequence with at least 95% identity to SEQ ID NO: 5 and the PAM sequence comprises 5′-CCN-3′ or 5′-NCCN-3′,
(f) the nuclease comprises an amino acid sequence with at least 95% identity to SEQ ID NO: 6 and the PAM sequence comprises 5′-CCN-3′ or 5′-NCCN-3′,
(g) the nuclease comprises an amino acid sequence with at least 95% identity to SEQ ID NO: 7 and the PAM sequence comprises 5′-CCN-3′ or 5′-NCCN-3′,
(h) the nuclease comprises an amino acid sequence with at least 95% identity to SEQ ID NO: 8 and the PAM sequence comprises 5′-CCN-3′ or 5′-NCCN-3′,
(i) the nuclease comprises an amino acid sequence with at least 95% identity to SEQ ID NO: 9 and the PAM sequence comprises 5′-CYN-3′ or 5′-NCYN-3′,
(j) the nuclease comprises an amino acid sequence with at least 95% identity to SEQ ID NO: 10 and the PAM sequence comprises 5′-CCG-3′ or 5′-NCCG-3′,
(k) the nuclease comprises an amino acid sequence with at least 95% identity to SEQ ID NO: 11 and the PAM sequence comprises 5′-CG-3′ or 5′-NCG-3′,
(l) the nuclease comprises an amino acid sequence with at least 95% identity to SEQ ID NO: 12 and the PAM sequence comprises 5′-A-3′, 5′-NA-3′, or 5′-NAA-3′,
(m) the nuclease comprises an amino acid sequence with at least 95% identity to SEQ ID NO: 13 and the PAM sequence comprises 5′-TA-3′ or 5′-NTA-3′,
(n) the nuclease comprises an amino acid sequence with at least 95% identity to SEQ ID NO: 14 and the PAM sequence comprises 5′-TG-3′ or 5′-NTG-3′,
(o) the nuclease comprises an amino acid sequence with at least 95% identity to SEQ ID NO: 15 and the PAM sequence comprises 5′-C-3′, 5′-NC-3′, or 5′-NNC-3′,
(p) the nuclease comprises an amino acid sequence with at least 95% identity to SEQ ID NO: 16 and the PAM sequence comprises 5′-CC-3′ or 5′-NCC-3′,
(q) the nuclease comprises an amino acid sequence with at least 95% identity to SEQ ID NO: 17 and the PAM sequence comprises 5′-CCNA-3′,
(r) the nuclease comprises an amino acid sequence with at least 95% identity to SEQ ID NO: 18 and the PAM sequence comprises 5′-CG-3′ or 5′-NCG-3′,
(s) the nuclease comprises an amino acid sequence with at least 95% identity to SEQ ID NO: 19 and the PAM sequence comprises 5′-AAN-3′ or 5′-NAAN-3′,
(t) the nuclease comprises an amino acid sequence with at least 95% identity to SEQ ID NO: 20 and the PAM sequence comprises 5′-AAG-3′ or 5′-NAAG-3′,
(u) the nuclease comprises an amino acid sequence with at least 95% identity to SEQ ID NO: 21 and the PAM sequence comprises 5′-AAG-3′ or 5′-NAAG-3′,
(v) the nuclease comprises an amino acid sequence with at least 95% identity to SEQ ID NO: 22 and the PAM sequence comprises 5′-TTN-3′ or 5′-NTTN-3′,
(w) the nuclease comprises an amino acid sequence with at least 95% identity to SEQ ID NO: 23 and the PAM sequence comprises 5′-TTN-3′ or 5′-NTTN-3′,
(x) the nuclease comprises an amino acid sequence with at least 95% identity to SEQ ID NO: 24 and the PAM sequence comprises 5′-GN-3′ or 5′-NGN-3′,
(y) the nuclease comprises an amino acid sequence with at least 95% identity to SEQ ID NO: 25 and the PAM sequence comprises 5′-CT-3′ or 5′-NCT-3′,
(z) the nuclease comprises an amino acid sequence with at least 95% identity to SEQ ID NO: 26 and the PAM sequence comprises 5′-TY-3′ or 5′-NTY-3′,
(aa) the nuclease comprises an amino acid sequence with at least 95% identity to SEQ ID NO: 27 and the PAM sequence comprises 5′-TY-3′ or 5′-NTY-3′,
(bb) the nuclease comprises an amino acid sequence with at least 95% identity to SEQ ID NO: 28 and the PAM sequence comprises 5′-TTC-3′ or 5′-NTTC-3′,
(cc) the nuclease comprises an amino acid sequence with at least 95% identity to SEQ ID NO: 29 and the PAM sequence comprises 5′-TY-3′ or 5′-NTY-3′,
(dd) the nuclease comprises an amino acid sequence with at least 95% identity to SEQ ID NO: 30 and the PAM sequence comprises 5′-GTN-3′ or 5′-NGTN-3′,
(ee) the nuclease comprises an amino acid sequence with at least 95% identity to SEQ ID NO: 31 and the PAM sequence comprises 5′-CCN-3′ or 5′-NCCN-3′,
(ff) the nuclease comprises an amino acid sequence with at least 95% identity to SEQ ID NO: 32 and the PAM sequence comprises 5′-CCY-3′ or 5′-NCCY-3′, wherein N is any nucleotide and Y is cytosine or thymine.

22. The gene editing system of claim 1, wherein the nuclease comprises the amino acid sequence set forth in any one of SEQ ID NOs: 1-32.

23. The gene editing system of claim 1, wherein the nuclease further comprises a peptide tag, a fluorescent protein, a base-editing domain, a DNA methylation domain, a histone residue modification domain, a localization factor, a transcription modification factor, a light-gated control factor, a chemically inducible factor, or a chromatin visualization factor.

24. The gene editing system of claim 1, which comprises the first nucleic acid encoding the nuclease polypeptide.

25. The gene editing system of claim 24, wherein the first nucleic acid is codon-optimized for expression in a cell.

26. The gene editing system of claim 24, wherein the first nucleic acid is a messenger RNA (mRNA).

27. The gene editing system of claim 24, wherein the first nucleic acid is included in a vector.

28. The gene editing system of claim 1, wherein the system comprises the second nucleic acid encoding the RNA guide.

29. The gene editing system of claim 28, wherein the nucleic acid encoding the RNA guide is located in a vector.

30. The gene editing system of claim 27, wherein the vector comprises the both the first nucleic acid encoding the nuclease polypeptide and the second nucleic acid encoding the RNA guide.

31. The gene editing system of claim 1, wherein the system comprises the first nucleic acid encoding the nuclease polypeptide, which is located on a first vector, and wherein the system comprises the second nucleic acid encoding the RNA guide, which is located on a second vector.

32. The gene editing system of claim 31, wherein the first and second vector are the same vector.

33. The gene editing system of claim 27, wherein the vector comprises a retroviral vector, a lentiviral vector, a phage vector, an adenoviral vector, an adeno-associated vector, or a herpes simplex vector.

34. The gene editing system of claim 1, wherein the gene editing system is present in a delivery gene editing system comprising a nanoparticle, a liposome, an exosome, a microvesicle, or a gene-gun.

35. A cell comprising the gene editing system of claim 1.

36. The cell of claim 35, wherein the cell is a eukaryotic cell.

37. The cell of claim 35, wherein the cell is a mammalian cell or a plant cell.

38. The cell of claim 35, wherein the cell is a human cell.

39. A method of introducing an indel into a target nucleic acid in a cell comprising:
(a) providing the gene editing system of claim 1; and
(b) delivering the gene editing system to the cell,
wherein recognition of the target sequence by the gene editing system results in a modification of the target nucleic acid.

40. The method of claim 39, wherein delivering the gene editing system to the cell is by transfection.

41. The method of claim 39, wherein the cell is a eukaryotic cell.

42. The method of claim 39, wherein the cell is a human cell.

43. The gene editing system of claim 1, wherein the nuclease comprises an amino acid sequence with at least 80% identity to SEQ ID NO: 26 or 27.

44-46. (canceled)

47. The gene editing system of claim 43, wherein:
(z) the nuclease comprises an amino acid sequence with at least 95% identity to SEQ ID NO: 26 and the direct repeat sequence comprises a nucleotide sequence with at least 90% identity to SEQ ID NO: 115 or SEQ ID NO: 116, or
(aa) the nuclease comprises an amino acid sequence with at least 95% identity to SEQ ID NO: 27 and the direct repeat sequence comprises a nucleotide sequence with at least 90% identity to SEQ ID NO: 117 or SEQ ID NO: 118.

48. The gene editing system of claim 43, wherein:
(z) the nuclease comprises an amino acid sequence with at least 95% identity to SEQ ID NO: 26 and the direct repeat sequence comprises a nucleotide sequence with at least 95% identity to SEQ ID NO: 115 or SEQ ID NO: 116, or
(aa) the nuclease comprises an amino acid sequence with at least 95% identity to SEQ ID NO: 27 and the direct repeat sequence comprises a nucleotide sequence with at least 95% identity to SEQ ID NO: 117 or SEQ ID NO: 118.

49. The gene editing system of claim 43, wherein:
(z) the nuclease comprises an amino acid sequence with at least 95% identity to SEQ ID NO: 26 and the direct repeat sequence comprises the nucleotide sequence set forth in SEQ ID NO: 115 or SEQ ID NO: 116, or
(aa) the nuclease comprises an amino acid sequence with at least 95% identity to SEQ ID NO: 27 and the direct repeat sequence comprises the nucleotide sequence set forth in SEQ ID NO: 117 or SEQ ID NO: 118.

50. The gene editing system of claim 43, wherein the RNA guide further comprises a trans-activating crRNA (tracrRNA) sequence.

51. The gene editing system of claim 50, wherein:
(z) the nuclease comprises an amino acid sequence with at least 95% identity to SEQ ID NO: 26 and the tracrRNA sequence comprises a nucleotide sequence with at least 90% identity to SEQ ID NO: 273, or
(aa) the nuclease comprises an amino acid sequence with at least 95% identity to SEQ ID NO: 27 and the tracrRNA sequence comprises a nucleotide sequence with at least 90% identity to SEQ ID NO: 274.

52. The gene editing system of claim 50, wherein:
(z) the nuclease comprises an amino acid sequence with at least 95% identity to SEQ ID NO: 26 and the tracrRNA sequence comprises a nucleotide sequence with at least 95% identity to SEQ ID NO: 273, or
(aa) the nuclease comprises an amino acid sequence with at least 95% identity to SEQ ID NO: 27 and the tracrRNA sequence comprises a nucleotide sequence with at least 95% identity to SEQ ID NO: 274.

53. The gene editing system of claim 50, wherein:
(z) the nuclease comprises an amino acid sequence with at least 95% identity to SEQ ID NO: 26 and the tracrRNA sequence comprises the nucleotide sequence set forth in SEQ ID NO: 273, or
(aa) the nuclease comprises an amino acid sequence with at least 95% identity to SEQ ID NO: 27 and the tracrRNA sequence comprises the nucleotide sequence set forth in SEQ ID NO: 274.

54. The gene editing system of claim 50, wherein the tracrRNA sequence is fused to the direct repeat sequence.

55. The gene editing system of claim 43, wherein the RNA guide is a single molecule RNA guide (sgRNA).

56. The gene editing system of claim 55, wherein:
(z) the nuclease comprises an amino acid sequence with at least 95% identity to SEQ ID NO: 26 and the sgRNA comprises a nucleotide sequence with at least 90% identity to SEQ ID NO: 444, or
(aa) the nuclease comprises an amino acid sequence with at least 95% identity to SEQ ID NO: 27 and the sgRNA comprises a nucleotide sequence with at least 90% identity to SEQ ID NO: 445.

57. The gene editing system of claim 55, wherein:
(z) the nuclease comprises an amino acid sequence with at least 95% identity to SEQ ID NO: 26 and the sgRNA comprises a nucleotide sequence with at least 95% identity to SEQ ID NO: 444, or
(aa) the nuclease comprises an amino acid sequence with at least 95% identity to SEQ ID NO: 27 and the sgRNA comprises a nucleotide sequence with at least 95% identity to SEQ ID NO: 445.

58. The gene editing system of claim 55, wherein:
(z) the nuclease comprises an amino acid sequence with at least 95% identity to SEQ ID NO: 26 and the sgRNA comprises the nucleotide sequence set forth in SEQ ID NO: 444, or
(aa) the nuclease comprises an amino acid sequence with at least 95% identity to SEQ ID NO: 27 and the sgRNA comprises the nucleotide sequence set forth in SEQ ID NO: 445.

59-61. (canceled)

62. The gene editing system of claim 43, wherein the target sequence is adjacent to a protospacer adjacent motif (PAM) sequence.

63. The gene editing system of claim 62, wherein:
(z) the nuclease comprises an amino acid sequence with at least 95% identity to SEQ ID NO: 26 and the PAM sequence comprises 5′-TY-3′ or 5′-NTY-3′, or
(aa) the nuclease comprises an amino acid sequence with at least 95% identity to SEQ ID NO: 27 and the PAM sequence comprises 5′-TY-3′ or 5′-NTY-3′.

64. The gene editing system of claim 43, wherein the nuclease comprises the amino acid sequence set forth in SEQ ID NO: 26 or 27.

65-84. (canceled)


SEQUENCE LISTING (496 sequences)

SEQ ID NO: 1 - protein, 492 aa, synthetic construct
MDEQKEAVIPKVARFRILKPVPPTTWQELGELLRAVRYRVFRLANLAVSEKYLQFHLWRTGRTESLDVRTVNQLNRDLRKVLEEEKEDEEAEEKKNQHDVDPARLSKTGALPDTVVAALSQYRIRPLTTGSKWSQVIRGQTALPTFRLGMPIPIRCDKPSHRRLERMQDGSVQLDLMVTRKPYPRVMLGTRNVGGGQAAVLERLLDNPVQDPSGYRQRCFEVKQDVQTGKWWLYVTYCFPAEATARSRDTVVGVDVGVSVPLYAALSHGHARLGHQHFGPLGKQIRNLQNQVVARRRSIQRAGRRGVVDKTARAGHGVRRMLGGTEKLRGRIDRAYTTLNHQLSAAVVRFARNHGAGAIQVEDLSGLQDTLRGTFLGGRWRYDQLQRFIEYKAKEAGIEYHKVNAAFTSRRCSECGVIHEGFTRTFRDQHGTQGRSARFECPACGYKADADYNAARNLSVVDIEERIRVQCAEQGLKAPTSAGEVDTEPEDL

SEQ ID NO: 2 - protein, 497 aa, synthetic construct
MGESVKAIKLKILDMFLDPECTKQDDNWRKDLSTMSRFCAEAGNMCLRDLYNYFSMPKEDRISSKDLYNAMYHKTKLLHPELPGKVANQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYTQGQLQIKKDHQGKWYCIIPYTFPTHETVLDPDKVMGVDLGVAKAVYWAFNSSYKRGCIDGGEIEHFRKMIRARRVSIQNQIKHSGDARKGHGRKRALKPIETLSEKEKNFRDTINHRYANRIVEAAIKQGCGTIQIENLEGIADTTGSKFLKNWPYYDLQTKIVNKAKEHGITVVAINPQYTSQRCSMCGYIEKTNRSSQAVFECKQCGYGSRTICINCRHVQVSGDVCEECGGIVKKENVNADYNAAKNISTPYIDQIIMEKCLELGIPYRSITCKECGHIQASGNTCEVCGSTNILKPKKIRKAK

SEQ ID NO: 3 - protein, 1366 aa, synthetic construct
MNRIYQGRITGILDSKEDERGHPPPPDPKHNPFWRHHEVYQAAVNYYYVAFAALGRAASDGMLRDLVQRVGESWETDPVHFRPEYGLRASLRQFLPLSDTSTLEEAFERILEKDHASPDTLLKAAMAVAKDLGGESSIQMKGREYLPRLCVPGYKGKFPREKNSLEKERMKVLLPVWLHAEDPASQPTLKKIRFHHFANPTGTRLEVAESKDLLGVAIDTLLRENKITAEEAGTLHDEIDALPDTFTLPAYAGGSVNKDALKLRFHAYLITEHFWKNGRGLELLRSTYPPPKGKPKKVGLSEEEEALLVDGDDPVKVARGDRGYVFRAFTSLPAWGGRSAKDIAWKEFDIAAFKEALTTYNQFKDKTEERAARLRDVQARLARMNGNEPEIPLAKEDKDDIPRLENDPRVALLGELLHDSGVVAEGQTVDRGIYHRSLRHYRDLRSEWNRVLSRASDGDSDSDISKRLISVVNEMQAKYAHTFGDVNLFRGLCSDPKYWPVWKHPDAETKKRIEKEGWAENVVEAYRDFLELQAEEERYSEAIHFRPAHPEESSRYFRFSDVVTKNQTLHEQPGERLKLPIVERNDEGVYTKSSIRVRYSAPRLYRDAVCGNGNEKGHWLQPMVKALSLPEPPEADITNSAIVLNIKTDGENSSDKRVRAYVDFPVELDTSPLKEHLGHTRKWAKQFNGQYDRGVPYPSAGGGLYWPDMQGLPSDPWYENPAIQASGFQVLGVDLGQRTAECHALIEIRCDGKFPQKKDGTSRDILATVGHDGTRTWEAVLLRAGTGRLPGENAHQMEQGRRVREKGGRRGRKTSDEDYRIAVEILEKIGLREIAESHRPLNDFKYAPELWDFVLRYLKWIRNRLGRLFFAAADLRDDELRDKVATRLEEYPIPGIELPSPLKERHEEAAELCLARYTELLETFRSALLLLTDQILPLRGRRWTWAPHPQFPESTHHILMETPEPSTKGRKIRGQRGLSFNRIEQIQELRKQFQALNRMEGWDIQAPHRPGRDEIRQSLPECCQPLLEKLDNLRTQRVNQTAHLILQEALGVELAPGEKNATGDEHGVYRVKHGRRPVDIIVLEALGDFKTSQKRGRGTNRRLASWAHRGISAKLRELAEPFGIPVVETPPHNTSTFHAFTGRPGYRAREMSGIEMERIQTKLEEKKTPKTFRETLIKAGIDAMRESGTPPEKMTLLVPQQMGELFLPLTGPDEPALPPIQSDINAAINIALKMVSAPEAVHLRHTVRFETPAGKPVTPGKGSKLEKALAKRKNVTFEIGDEFHAPQARIDRANLLGTPAGTVHTPIGSFDFMSRDAMRNLLSHYKDDMFIQANKRALEKRGISFPMTPSNHPFSAGDFMDEHFPM

SEQ ID NO: 4 - protein, 1360 aa, synthetic construct
MATRIYQGRIVQARFEDENLPQDKESALAALERTNRLFQDAVNYHLVALAGMAEDGKETLGSRFKKQVRAIWEDLPRSKVGACTLQRSIARTLSLADGVTFDDAVAHIYEGCDRLDVLPYVEHYVIEQTQKGEGAIQQQGRELLPKLCNSDFEGNFDYSIKERKANSGKQKLLRELNRDDVSDEELYALAQEMDLSWVVKTIPNENHDEPLYYDPEEQEEKVITSIHKLLSSLDNGNLKIATLPKYVQIDGFREQMRQDLLNRMPLKGLRLAKSSRGISVDVEQAGIYFMFYPGRISAAHLASKLGKEKPKKTDEVREYDCFSLENDPLILARGKRGYVYKGFSALPNWESSDNRMYSKEWDILAFKEALKTLHGFELKTKERDAERAKYVAQLEYVERGKKTKDYVEPDEEKTVAVLGGDYRFELLKKLVAEISPDPVTEYHISSRTLNDYEEVRALWLKRELTGDCSSHDLSKIVREYQAKSKRFGSQVLFAALCQDEYRPIWHDYHALKDEKLPRSKNILRDFSNWQYLCGQVEKYSRDVRVTAADVVASPRQMIYSDLSNFGNGKGCEYIPGCEGGLRMQVVVRNAKGHWETDSIRVTFSAPRFLRDEMGQDAGKWMVPKKGENTTLPWLQPMMKALGDDIAPVRLERTPAIGLQVWGQGSEACYYLNFPVSLDVSALQKSLGKAARWAGQFLGGRDEKLHLHWPATYKGKNRPWWEQEKEFTVLGIDLGLRSSVAWSLLRVSTCSTSSNSRGDELIGRLIGDSSNAKWYGYVIKQGLSRLPGEQPRRVGDKKIPAVSLASPEDRRIAKLIIEAAGAQFDENESEDVLRLGNRTLKSFKSLTSRLKTYLSFLSGLKDPGRKPAVLKRMADYFAYAEIIPGVCVLLEAHQEDEVYERVLDAAEELRSMLPKYAERVTSLILPRKHGSWKWEPERRKGWQGSGVMRLTEEDGIPHRPVFHRGGLSVARLTQLETLRQLLQSMGKVLSFVPGESVTFGRRLKDEKVIDPCPEILEKIENMREQRVNLIAHDIVAQALGVRLKSSRPHKNSGGLDVIHGEYERIHGREPVDFVVMENLSRYLTSLDRAPLENSGLMRWAHRQIVAKVTQLLEEIFGIPVVFTHAHYTSKFDSMTSEPGFRPVMLKPEYLKWLQRNGKGNECKAAAVYQAIWDEVVNASKTKKVTLVLPHLTKGGELANGGELFLSQKGGKFTLRNADMNAATNVAWRGLAAPESLHLLHRVRMEMKKAGFVPVCDNAREKSLKTGWSLTKLKSIQPEGNKISAFAVSSEWKDEYFAAYGNAETRAYLAYGKTLWGIMKRKQWQMCHLFNIQQLKNAGIDARLVEQLLHTDENDTSDDIPT

SEQ ID NO: 5 - protein, 546 aa, synthetic construct
MFKTARFKVHNPSRHKSTMLWYAMTRYHNTLKDVLEKTLAIPNLLEQVSELDKKEKLRPNKFKLSKLLYTIAPQNWELAPLRDYLIGDASAMLMSHLNKTYKGANESNPPTVSSLNAMTDAEFHKAYSDFTDPEAKLAIKPQHQEKIDKAAERGETNVANRLSKIYANWAASRAAGQVLRKLEGELPHPIEFTRNEIGRGCLLAYCDGNYYLLVRLFAQDHRYCEKRVLKEGFINCKTKEHIEGKKYPGMILPLELGREFHEREYLTHGSIQSAKLIVKRREKPNSQPKALDSKPAPFNAEDYDFYVHAAFEFQPTQIETETFLGIDRGAAKIGAATLIDRQGKPLETDLDLSGAAFAAEMRRFEQQIKRIQKQGKQKSRKFSLRGKRADIILGEYANRIVAIAKENRSQIVIEAIKGVTMGRFLKQSQFTKLKQMLTYKAEREGLPAPIEVPAAFTSQTCARCGHKDPANRPKKDAAGKAIQDVFLCTACGHHANADSNASLIIALRGLHQIENGGKFKKFDLFQQWLKEIIGRDGSFAPGQVSP

SEQ ID NO: 6 - protein, 526 aa, synthetic construct
MFKTARFKIHNPSRHKQAVLRYALSHYHLTLKNVLEAALADPELQARVTVVGKNGKPRTDKAALSRFLYALAPKGWPLAPLRDYLIGDASAMLLSHYEKDLKGKNESNPPTLGGLEGLTEERRREAFHDFVMTEEFPLRADRAAEIEKARSLGQVHLAKRLGNIYASRASANAMRDLLRSLDAPIPRPIEFTRCEFGRGFLLARKGDKFYLLLRLFSKGNSYWQQVMLDEGFVNWKTKETIGGRKYPGVVLPLEFGRDFHESEYLEHGSPQSAKLILKRSDEGLEEFCAHIAFEFTPEPVTPETFLGIDRGAAMIGAATVIDQAGGLVTRRLDLEGTAFNRELKRFEFRIAKAQRKAQRRPRLFRVRRRWAAIVIGEYANRVVAEAVKHRSQIVLEKIDARSMARFLSHSQFRKLHDAITYKAERMGLPRPIEVPAAYTSQTCARCGHRDAANRPKKDSEGRPLQAVFRCVRCGCEANADGNASEVIALRGLHQALNGGKFQKFPAFQEWLVGIRRRVGEPALIGR

SEQ ID NO: 7 - protein, 594 aa, synthetic construct
MFKTARFKVHNPSRHKSTMLWYAMTRYHETLKAVLEETLAIPDLLEQVSELDKKEKLRPNKFRLSKLLYTIVPKSWELAPLRDYLIGDTSAMLMSHLSKAYKGENESNPPTVSSLAAMTDEEFRKAYSEFTDPEAQLAVKPQHQEKIDKASERGETRVAKRLSKIYANWAVSRAAGQVLRRLEGALPHPIEFTRNEFGRGCLLAFCDGNYYLLVRLFAQGHRYCEKRVLKDGFIDCKTKERLDGKRYPGLILPLELGREFHEREYLTYGAIQSAKLVVKRREKPASGSKQAAEKPAPEGGGGFNPRIKPTEPKLALATEERFPPIPPEIPSFSAASKALALQPIPFNAGDYDFYLHAAFEFNPPKVETVTFLGIDRGAAKLGAATLIDRMGKALETNLDLDGDAFKAEMRQHEEQIIRLQKLGKQRSRKFSLRGKRADMILGEYANRIVAIAKQNRSQIVIEAIKGTTMNLFLKQSQFTKLKQMLTYKAEREGLPAPVEIPAARTSQTCAKCGHWDRANRPKKDAAGKAIQDVFLCTACGHRANADSNASLIIALRGLHQMENGGKFKKFDLFQQWLKELIGRDGSSAFGQGNQ

SEQ ID NO: 8 - protein, 553 aa, synthetic construct
MFKTARFKVHNPSRHKSTMLWYAMTRYHETLKAVLEETLAIPDLLEQVSELDKKEKRRPNKFKLSKLLYTIVPKSWELAPLRDYLIGDASAMLMSHLSKAYKGENESNPPTVSSLAAMTDEEFRKAYSEFSDPEAILAVKPQHQEKIDKASERGETRVAKRLTKIYANWAVSRAAGQVLRKLEGALPHPIEFTRNEFGRGCLLAFCDGNYYLLVRLFAQGHRYCKKLVLKDGFIDCKTKAPLGGKKYPGLILPLELGREFHEREYLTYGSIQSAKLVVKRREKPASRAKARAAGDALKGGQPVPLNAEDYDFYLHAAFEFNPAKVETETFLGIDRGAAKLGAATLIDRMGKALETNLDLDGDAFKLEMRHHEAQIIRLQKLGKQRSRRFSLRGKRGEIILGEYANRIVSIAKQNRSQIVIEAIRGVTMGRFLKQSQFAKLKQMLTYKAEREGLPTPVEVPAAYTSQTCAKCGHKDPANRPKKDAAGKAIQDVFKCVACGHTANADSNASVIIALRGLHQVENGGKFKKFDLFQQWLKELIGRDGSVALGQGNQ

SEQ ID NO: 9 - protein, 544 aa, synthetic construct
MFKTARFKVHNPSRHKSTMLWYAMTRYHETLKDVLEKTLAIPDLLEQVSELDKKEKLRPNKYTLAKLVRTIVPKNTGLASTVRDYLIGDASAMLMSHLNKVYKGANESNPPTVSSLAAMTDEEFRRAYSNFTDPEAKLIIKPQHQEKIDKAMEQGETRVAERLSKIYANWAISRAAGQVLRKLEGTLPHPIEFTHTEFKRGCLLAFCDGKYYALIRLFADKHRYKQKNFLNSGFIDCKTKESLGGKEYPGLILPLELGREFHEREYLTHGSIQSAKLLVKRRVYSNSQTKAPDSKPTTFNAEDYDFYIHAAFEFQPTKVETETFLGIDRGAAKIGAATLINLQGKLLETNLDLEGSAFASEMRRFDEQIKRIQKTGKQRSRKFSIRGKRADIILGEYANRIVAIAKQYRSQIVIEAIRGVTMGRFLKQSQFTKLKQMLTYKAEREGLPAPVEVPAAYTSQTCARCGHKDAANRPKKDAAGKAIQDVFLCMACGHKANADSNASLIIALRGMHQKENGGKLYKKFDLFQQWLKELIGRDGSAAPG

SEQ ID NO: 10 - protein, 561 aa, synthetic construct
MFKTARFKVHNPSRHKSTMLWYAMTRYHETLKDVLEKTLAIPDLLEQISELDKKEKLRPNQYKLRMLLRKILPRGWELAPLRGYLELDASAMLMSHFNKAYKGANGSNPPTVSSLDPMTEKEYHEAYKEFTNPEASLSIKPQQQEKINAASERGETRVAKRLSKIYANWAISRAAGLVLRKLEGTLPHPIEFTNNEFGRGCLLAYCDGNYYLLVRLFAQGNHYCKKLVLKDGFIDCKTKELLAGKKYPGLILPLELGREFHEQEYLTYGSIQSAKLIVKRREKPTSAAKARIHNTGLAARLKSCPDTKQTASFNAEDYDFYVHAAFEFQPTQIETETFLGIDRGAAKIGAATLIDREGKPIETNLDLEGSAFASEMRRYEEQIKRIQKTGKQRSRKFSLRGKRADIILGEYANRIVAIAKEYRSQIVIEAIRGVTMGRFLKQSQFAKLKQMLTYKAEREGLPAPVEVPAARTSQTCARCGHWDRANRPKKDAAGKAIQDVFLCVACGHKANADSNASLIIALRGMHQKENGGKFKKFDLFQLWLKELIGRDGSIASGQGNQ

SEQ ID NO: 11 - protein, 554 aa, synthetic construct
MFKTARFKVHNPSRHKSTMLWYAMTHYHDALKAVLEKTLAVPDLLEQISELDKKEKLRPNQYKLRKLLRTILPRGWELAPLTRYLELDAAAMLMSHFNKSYKGANESNPPTVSSLDAMTDAEFRKAYSEFTDPEAKLTVKPQHQEKIDKAKERGETRVAKRLSKIYASWAVSRAAGQVLKKLEGALPHPIEFTNNEFGQGCLLAYCDGNYYLLVRLFAKKHRYKQNNVLKDGFISCKTKEPLGGKKYPGLILPLELGREFHEQEYLTYGSIQSAKLVVKRREKPASRAKARTSSRISTDKQKPIPFNAEDYDFYVHAAFEFNPPKVETMTFLGIDRGAAKLGAATLIDQHGKRLETNLDLDGSAFAAEMRRYEQQIKRIQQQGKQKSRKFSLRGKRAEIILGEYANRIVAIAKENRSQIVIEAIKGVTMGRFLKQSQFTKLKQMLTYKAEREGLPAPIEVPAAFTSQTCARCSHKDPANRPKKDAAGKSIQDVFLCTACGHHANADSNASLIIALRGMHQIENGGKFKKFDIFQQWLKELIGRDGSSAPGQLNP

SEQ ID NO: 12 - protein, 1166 aa, synthetic construct
MKDIKHSLISGQKAQKFRKHLAMSSASASVKKNKTSILTYKFRLDLNENMSEIQDRIPKFSDYIKLYNKIEGVEPGTLTHYLCTFVLAGFRLFSNAKSAFEFIKSQNNPCLEHLSSHKLLKSSAVAFDLTANLAISEPGYEPYLAIARILERYTDPDKKINSFVKDNFTTYNNNALSWLLGKGHKFFKESTAQEIALYYGIPDYKFDCAKAIKNAADKLEFNSSLFSNDMRLSQFRSCFGGHIDSWATNYIKRLLELEKIIANISYEIKIPKAFISSSNDFLTHCNLNRDDIEELISNIKSSSTITDVKDALSTLLGHKQGASSADIKAIRDYSELINRLCAYKEQIFNTIDQAAEDKNSLWHDIRRQTKDELQTWEKLEKLPKLNDLSGGVPQAENELNAKLMQLKLVTEAQNNHFAKIMQWVHSNIKDFSPFNHIVQTEQEKLDNRPKENTTACDLAVRMFLHKVGRIAREDNNNLCKELQQWFLDNKVFDNKTDFNKYFHNKLGSIYISPYSTQKNAGYKINKEVLNFGEKIVLLFTDKLQEINKRYEGNSIAEKSELNSLLKLNYFYYNFFISGINKAVPVSIVKPLLPDDMLEQSLSATHKIRLKSNEVDPSSLSSIFNIYKSLISGCYTVLNRETFFLRTKFSWIENFTLFYVPKADASWIMPKRYLKNTRWQQYIEEEVLVFENDKYKVDITQTFNNICSAPADYAELLVQLPHDWFYQLPYECAKEDNYVQALAICKDKGFPKQSRLNTHISGRLIGPSSFKSKLDSVLIYNGDVTISDMTLLVEQRVSQQLKPDESLELKKYDPEFTLAIPINDARSQSTNYSFKHIIAIDQGEIGPSYAVFNLSDAGNANAEPIATGSIRIPSIRRLIKSVSSFRKKKSTTQKFNQRFDSTMFNIRENVTGDICSVIVGLMQKYNAFPVLEREVSNLESGSKQLSLVYKAVNSMFLYSDVEMQNTNRKSWWKNADHWQTNILRLIRGENKTSKSVKLNGQNYKELKIYPGVSVSAYMTSRICSCCGRNIFELIKNDELEDKHKKYQVNAQGEINIRGEVIKLYQKSDSHKTLVPGLKSKKTYNAINQRAPMVTPYPEGIIDIEQLKKIIRFNLRRAPASRMSKDSTQSRYFCVFKNCKNHQVEKHADINAAINIGRRFLTDIIIHN

SEQ ID NO: 13 - protein, 1262 aa, synthetic construct
MLTTKFKLELPAGCPLREDAATFDECRKLYDVVEGCGNGTLTGFLFSVILSGFRIFPDGKMAEIFANRSVYDEDEFRSALVEAVGAPLPRFTVKALIKRLQMEVRARGNKDNRFVAEVMMKEYRQTLCGKTLPKGVDESYVDRLFEEMARELTSRYRSWNELKGDLLGACKAVDAALRGFGDFPSLATMVTRAAARRLPKDSTIVFDPQSPFIDVQTIGVDAMPYAAVATLLSYPESVGEKRRDFVQNHLTTPSAAGLSWLFNRGLELFSEESVEELCRLFHVPEDQRTRIVQIQNAARATPRQSFFLKKGGAPLGYHDFRSAFAGRINSWTANYLNRLEELQGLLHDLTDELRLPDLVRNGEDFLATTDCRREEVEILCRSFSRERDRAQTAVEHLIGADPLQVVSDVAAIEEYSRIVNRLCAIKEQIVNSLRQAEDDKASRWTTLWSEVKNEFQPWEKLIRLPKLNGMSGGVPPAQDELETILARYSDVVRGATEHFDVVMEWAAKTGAEGDILKKFAETEQQRADQRAPGKYDGRELALRLVLQRVARVVRDRSDVCAENVRQWFLKENIFAERKDFNKFFFNRLGSLYVSPFSNRRHVGYKLSDGLVERSGAVWRELLALVKEKRGAYEAFSEAGETFLRLENLLMVMRIGALTENVPAEVAALRLDEETALESVSEGLKLQLQQAEVPPSVLAKAFNVYVSLLSGCLIALRRERFFLRTKFSFVGNTALVYVPKEKSWPMPSRYEASPSWTPIFENDVLVRLSTGEVEVAETFRRAAALWGRTTDPVLKKALRELFHQLPHDWCCQVSVRSSGDMTPAKRKEDDRDVLIVEKKGKYDSTIISKKIAATALVRLVGPSTHKERLNRLLLDVGEVACDMTLLADQEILQKVEDGRVILSLGKLQFSLSVPISTPAEQSEDEVKSERKSTHFRRIVAIDQGERGFAFAVFRLEDAGKEGAQPIAQGFVNIPSIRRLIARVHSYRKGKQSVQKFSQRFDSTMFTLRENVAGDVCGAIAGLMCRYRAIPVLERQVSNLASGGKQLELVYKMVNARFLDDRIPMHSLERTSWWCGTSDWVIPDLWVEVPESYAVKAKKDEILKKDGKFYRTLRITPGVGVNAKWTSRICSQCGGNAMELIEKAREEKVKTVTLDANGEVTLFGRTVRLYKRPSEERSREARRRNERAPWTEPRANVKLSLDDFRRAVAENMRRQPKSLQSRDTSQSRYFCVFTDCRCHNKEQHADINAAVNIGRRFLESLLRE

SEQ ID NO: 14 - protein, 1302 aa, synthetic construct
MQTKKTHLHLISAKASRKYRRTIACLSDTAKKDLERRKQSGAADPAQELSCLKTIKFKLEVPEGSKLPSFDRISQIYNALETIEKGSLSYLLFALILSGFRIFPNSSAAKTFASSSCYKNDQFASQIKEIFGEMVKNFIPSELESILKKGRRKNNKDWTEENIKRVLNSEFGRKNSEGSSALFDSFLSKFSQELFRKFDSWNEVNKKYLEAAELLDSMLASYGPFDSVCKMIGDSDSRNSLPDKSTIAFTNNAEITVDIESSVMPYMAIAALLREYRQSKSKAAPVAYVQSHLTTTNGNGLSWFFKFGLDLIRKAPVSSKQSTSDGSKSLQELFSVPDDKLDGLKFIKEACEALPEASLLCGEKGELLGYQDFRTSFAGHIDSWVANYVNRLFELIELVNQLPESIKLPSILTQKNHNLVASLGLQEAEVSHSLELFEGLVKNVRQTLKKLAGIDISSSPNEQDIKEFYAFSDVLNRLGSIRNQIENAVQTAKKDKIDLESAIEWKEWKKLKKLPKLNGLGGGVPKQQELLDKALESVKQIRHYQRIDFERVIQWAVNEHCLETVPKFLVDAEKKKINKESSTDFAAKENAVRFLLEGIGAAARGKTDSVSKAAYNWFVVNNFLAKKDLNRYFINCQGCIYKPPYSKRRSLAFALRSDNKDTIEVVWEKFETFYKEISKEIEKFNIFSQEFQTFLHLENLRMKLLLRRIQKPIPAEIAFFSLPQEYYDSLPPNVAFLALNQEITPSEYITQFNLYSSFLNGNLILLRRSRSYLRAKFSWVGNSKLIYAAKEARLWKIPNAYWKSDEWKMILDSNVLVFDKAGNVLPAPTLKKVCEREGDLRLFYPLLRQLPHDWCYRNPFVKSVGREKNVIEVNKEGEPKVASALPGSLFRLIGPAPFKSLLDDCFFNPLDKDLRECMLIVDQEISQKVEAQKVEASLESCTYSIAVPIRYHLEEPKVSNQFENVLAIDQGEAGLAYAVFSLKSIGEAETKPIAVGTIRIPSIRRLIHSVSTYRKKKQRLQNFKQNYDSTAFIMRENVTGDVCAKIVGLMKEFNAFPVLEYDVKNLESGSRQLSAVYKAVNSHFLYFKEPGRDALRKQLWYGGDSWTIDGIEIVTRERKEDGKEGVEKIVPLKVFPGRSVSARFTSKTCSCCGRNVFDWLFTEKKAKTNKKFNVNSKGELTTADGVIQLFEADRSKGPKFYARRKERTPLTKPIAKGSYSLEEIERRVRTNLRRAPKSKQSRDTSQSQYFCVYKDCALHFSGMQADENAAINIGRRFLTALRKNRRSDFPSNVKISDRLLDN

SEQ ID NO: 15 - protein, 1455 aa, synthetic construct
MKTENRGLMYSAPFIKLVEGNLMTSNETTTQSKDFKNYRHPRFNPKKDGSLKKVIQIVKDGVPTYGDFNPHEQKKDKTPNRRGHLLSRGESRKLKRSIRALSDSQKKGEGAFEQTRTIVCKTKGDVRPEANFELMRETYNTLNALDFGSLKFSFLALPFLGMIASPKTAQCVINENREHFVPDGLFESIVKDLGVLGEGMTPETIALAVQAEPGKKCCPDKGRVEFKKVLFRAFPKSMGFSLEYVINQIKGLTAELPKLESEVAALKSEVAQIEKDLQELSEESKKYQRTEKSLLKKESDLKTKELKLAEYEQKLTNYKAERDSFFKVDDFIQEVLDNVVACSEVKDRADFLNLDKKVYVYECFDLALKKLNPEYSGRLTSLASFFKNHKPKGRTIAFVPDLEYKGMDFLKDNADILPYLNFSALMNRLVSLGLLKRGEFSPKVITTFNDLVLSPNNDALNSFLGVGFEKIRTSSLEELRDYFNVESDKDDTIKALQELLTLAVEDHVFGKKSYSEFRVEVGSQIKSFYSNHGARCIAFYNASIDPSPIEVSGDLWNLSNAYLFRKTYANPEFLKNQIESVNSSGEVLKSRLFRIMGYSEELPSREDIEFFKDFSRLVDRTFSDIERINTSLKEEIKTYDPKTDKKIIKSLEDLIIITPKWAKDIKKLAGVGGGKKSAKEEAQDLIDRFNLIRSRLNTQVDAVVVTAGNLETMEVHKQVSLDALKSNLDYDSTVDYDELYYRETFDSLIRLIRDTNCPPLIKKFRDDVVSYGLVRGSGIKPLTVYINSGKGRVFVHPKSNYKHSALNIELSILEKFNPVAYLEGLLEYLDSIPRLKPGRLFCLKETIRLEIIKFKIFNIPDTVPVSSINQDYFDLIEGRTFLSDITRDKDEILNSEFRTIINCYITTLRSIIPDVTQEGVSLRLTFRKKGTSTVMGVPKHEEVTTNEKGETESIFKFTLPASLEYSKGPLSTLISELKNPSSVFKVESKVTKESFPKIEVSVGKGHQVVKLSERSIKKLTSPELVASDGKKIPNPYLEGVSQLLNYIPQNLCIETGWKVSKSDKDTRGITFGKKKLGFKSAPGVLKLVSGATQLQTVQQSLIDEDINLGDVEYVFEQKYKKKLDFRGDQVFLRNVKIEHSKNKPEVEVFLNIPVTEKRLIKKSAISPFNHTADIGFDLGEYGLAYAVLDIRTGEIKATGFVPIKMFRKLINVVNSYRKHNQPRRDYSKFSDSKLQNMKEAATAEICTIIWSLMDLHNALPVFENNVSGLSRGKNAVRNIYANVVDYFVRNSSNAASQSRLKHSFYGDIKITRTDGKGKKIFYSPGRVVSGAYTSQECSCCGKNPVRMVRYSDVEEYSIDSEGAVILDGEFKYYLKATAKNNTSKHTRADFTAAFKPGGKIRKKDLISRIKLQMRRAPVDKRTKNSSQSRYVCLFDDCSLVEMSADTNAAINIVKR

SEQ ID NO: 16 - protein, 479 aa, synthetic construct
MIKTFKTAVFNVKFSKRKGNIIDTQMRLAENAFYDVIERLAHHVEPLIKLNKEQRKDMLTRLKKEASQLIKPHPLSNASKSGVVADAIAQISSTVELRLTGQDAKLPTRNNRDIDTYDIGMDMLVGSLDLESQDLAKQLIYSKPYDGMPRPLLWLRTRPSDGAMLLRDGLGRYFVYINSHSSKSKFSKAKVVINDLVNVRTGETENFSSSTGLLLPIQLSKWHQSEFLAKGKPKSYRLIKKADGYILAVTFEFKAEKIEPATYLGVDRGIDKIAAFAVTSKKEVLKKDFCDGNELRDYQKECETNARKKQTKGNAKYIRWRGYTDLIMHKIANEIVNTALKYRSQVVLEDLTNIANGHHHRRARFARKTNFNKVLSRQQYQKLQHLLNYKLSYVGLPTPLFVRAAGTSITCNRCGNYDSKNRDLNERSLFLCKSCNYQDNADVNAAVTISMKGEWLTTQFDKEHKKMKNRFSDWIPLPS

SEQ ID NO: 17 - protein, 506 aa, synthetic construct
MSDVVTSFLTVKYKLHNPSKRRRAMLLDAMRRAHLGYDKLLKRVREDVEAIVDITERQERTDAEKELTKKLQALAKPLPLGNGPKQAIIADALAQSKSYVELKKADPNTSYPTTPRLKVDQVDYDAAVDGIANSQSILEENEYRDLLAKLSRPGLPRPLNILKNRIGDGALLLQDDNGRLFVFINLLPKTAKRKRKVDLTGLIDTRTGEIMQKSTSSGDIFPLECGKWHDEKFLKQGTLQSSRLIYDGKDFYFAATFQFEAPLREPTNYIGVDRGIELLAAWSVIDDKGRKLDAGYHGGERLRSFQRRQEQDQKDTQRRGKIYTSRTRRAVADEEVHIVANKIVDMAAKHNAVVVLEDLKTITMGPHQKRPKGARKSGFRRMLTRAQYAKLKHCVDYRLKMEGFAPLRRNSPSYMEIHPAYTSLTCAKCAHQDKESRQSQAVFVCTKCGHKDNADENAAVNVAAKGIHFDQIVKGRKKGQKLKDHEQFSAWYADLKNGGGGHADGP

SEQ ID NO: 18 - protein, 481 aa, synthetic construct
MKATTITKSLRFWIDEPPERCRLLYGVSDELTDAYNAILEYWETDVRKVAVEATEAAIEAYHVWKDAPKETRGDKPKWWSADTATRAAFLRVGVAATSTIRTRLVDNMVQGEWLRDVKTYMSRRFKDHIAAGIRHRAKGVVISLDQSTMKPDVQLGDDGRWTIRCALWAQGTEESARNNDRWLLSPYDRKRQTWTIANLLEADQHGEVRGVKLVPPKPGAPAGKRRWSAMITVTLPVESSVLDEERPNVAGVDMGLTHFAVYSCPARNTFEFVSSRELQAAMEKARRRRRGVPRKRSTALGQKLGRRQDALCRLTARRLIDCCRRDRVGTLRVEDLTGIRDQGSDDADRNFALGARFPYYKLQTYLEQAAASAGVRLEKVQPAGTSQTCSRCAVRDPESRDGKRFVCRHCGYKGDADLNAANNIASGRFRRSIRPSTQPAGRIPSAAPDCTGEAVKASDVDAELQPARTPAACGGDPYAGE

SEQ ID NO: 19 - protein, 387 aa, synthetic construct
MLKAHVIRLNPTEEQASYFWRCAGIARFTWNWALAELNAAYDRGERPAIGSLKLAFNRLRKEEGFAPFVGEVQSYAYQQAFTDLQKALSRYHDFRKRGLLKPPAGWKGRKDHKPFGWPRFKARNRSTPAFYLANNGGLRLQGHQVTIQRCPGGPVNMAEQLRFAGRVMGGRVRYRAGHWYLTVQVDVPVEPVPAHTGPAVGLDVGIKTLAVTSDGEIYDNPKALGRHQRKLRLLQRSLARQTRGGSNYRKTQAKIARLHERIANIRKHTLHQISHEITRDYGLIGLEDLNVAGMLKNGKLARSISDVAFGELRRQIGYKSEWRGSRVVIVSRWFPSSKTCNECGHVMADMPLSVRWWQCPTCGAEHDRDGNAAVNIRNEAVKMAGAA

SEQ ID NO: 20 - protein, 387 aa, synthetic construct
MLKAHVIRLNPTEEQASYFWRCAGIARFTWNWALAELNAAYDRGERPAIGSLKLAFNRLRKEEGFAPFVGEVQSYAYQQAFTDLQKALSRYHDFRKRGLLKPPAGWKGRKDHKPFGWPRFKARNRSTPAFYLANNGGLRLQGHQVTIQRCPGGPVNMAEQLRFAGRVMGGRVRYRAGHWYLTVQVDVPVEPVPAHTGPAVGLDVGIKALAVTSDGEIYDNPKALGRHQRKLRLLQRSLARQTRGGSNYRKTQAKIARLHERIANIRKHTLHQISHEITRDYGLIGLEDLNVAGMLENGKLARSISDVAFGELRRQIGYKSEWRGSRVVIVSRWFPSSKTCNECGHVMADMPLSVRWWQCPTCGAEHDRDGNAAVNIRNEAVKMAGAA

SEQ ID NO: 21 - protein, 387 aa, synthetic construct
MLKAHVIRLNPTKEQETYFWRCAGVARFTWNWALAELNAAYEKGERPAVGSLKLEFNRLRNEEGFAPFVGEVQSYAYQQAFGDLQKALSSYHDFRKRGMLKPPTSWKGRKDHKPFGWPRFKVRNRSTPAFYLANNGGLRMNGHQVTIQRCPGGPVNMAEPLRFTGKVMGGRVRYRAGHWYLTVQVDVPVEPDPAHTGPAVGLDVGIKVLAFTSDGVIYDNPKALAHYQRKLRLLQRSLSRQTRGGSNYRKTQAKIARLHDRIANIRKHALHQVSHEITRDYGLIGLEDLNVSGMLKNGKLARSISDVALGELRRQIEYKADWRGSRVMIVSRWFPSSKTCNDCGYVMADMLLSVRWWQCPACGAEHDRDGNAAVNIRNEAVKMAGAA

SEQ ID NO: 22 - protein, 512 aa, synthetic construct
MYGDSRIEEGNMDALGKSSRDGTNQHPPCDAASTAVCETAVIVRATARMTIDFMAMEEPERIRARQLLYEARRNSAAAANAVLRAFWRADGDALDGYMVEHGHGPKKAVDWPMPKIQSYGLARLVAPMLPSGIGSAVARMAETKWRQTRFEALVRNTEKPAHYREANPIPIRQQDYSLTKEDGRWCLSMMLRSGEPRVKLPLDIRDSFQRSILENVSEWKGSRALARKKGESREAYKQRVASLEAAQRGWEAGELRIEQDSKRKARWYVRMAYKRIVDKQADGKRASLHRGIKNFLVCVTDDGQEWKYEGADIEAFLAQMQARRRQYQRNSLASSRSGHGRRVILRPIDKLQGKADRWRRTKNQTLARRLAEWLRDRGVSVLFIEDLSGIRSGEPEKLEGGERIYKRVQEWPFYDAGQRIHSCCEELGINVKTVSPAYDSQRCPVCGAIDPEHKDLRYWKLSCKSCGARRDLDVAAAYNVLARGLAVHGGKGEDYKDIGRAKRAAKGKKNAG

SEQ ID NO: 23 - protein, 512 aa, synthetic construct
MYGDSRIEEGNMDALGKSSRDGTNQHPPCDAASTAVCETAVIVRATARMTIDFMAMEEPERIRVRQLLYEARRNSAAAANAVLRAFWRADGDALDGYMVEHGHGPKKAVDWPMPKIQSYGLARLVAPMLPSGIGSAVARMAETKWRQTRFEALVRNTEKPAHYREANPIPIRQQDYSLTKEDGRWCLSMMLRSGEPRVKLPLDIRDSFQRSILENVSEWKGSRALARKKGESREAYKQRVASLEAAQRGWEAGELRIEQDSKRKARWYVRMAYKRIVDKQADGKRASLHRGIKNFLVCVTDDGQEWKYEGADIEAFLAQMQARRRQYQRNSLASSRSGHGRRVILRPIDKLQGKADRWRRTKNQTLARRLAEWLRDRGVSVLFIEDLSGIRSGEPEKLEGGERIYKRVQEWPFYDAGQRIHSCCEELGINVKTVSPAYDSQRCPVCGAIDPEHKDLRYWKLSCKSCGARRDLDVAAAYNVLARGLAVHGGKGEDYKDIGRAKRAAKGKKNAG

SEQ ID NO: 24 - protein, 858 aa, synthetic construct
MKHQYKPKKCKFIEHRAVKFDRETGNPKLDASGAEIPFTENRTAVCKINPKSVDPRLLETFDASKETINDILANMSEHWFDVYTVESGVKNDMKKFTIMDLYAGAVPGDILKGEFTLVHGRKRVLVKKTITGYVTRELMAPQEDDGFILCDREQFINSLNRKTDKIFGEETSIPAKWWCDTICGDLDTMLKGYAQCVLGMSDTDDGKWRTAVREVSESIYGNEFSRKHAERTIIKLGPQHLRHVNGLMPDTSVIQWPISCKICGENATITEPDFAKEPKLKRLYLASMKAFERIVKESFPKKNVFKPNIPMLPRDSVKKLDGYYNYSAELLYIPGPKKASRFRVEFRAKSDRTGNDYYPKDLFKYTSECIIPRFSMLKSTGAMTLNIPYTVPCQKPFMSQDAEINWDAGLGIDLGYARFAMVLSKPASKYPGMVNWNEALDWFSKKYGLDVLNAHCSKATRKEIEDMIAEERDGKATMGAIFLLGVRDGNPPDIQHDWRPSHDPMATLFTRMERRTDKDGSPFYSEQQLAIIGHTKTFRIQMRQIFANRIEYYHRQSEWDLNHSEEQVFARESEVAKALAARYDFLNESIRCITQRFISDILTSDGAFRPAFIAMEDLNLNELEKDSSFKSLYMTITGDWGIDPRQDYKVSVRKGRTVAEITYPEGKKPPRPAQFPKVFPATEHWNTPARISAKGQTIVIACTPTSKGTVAMARDSIECYTKKALHIALIKHDVERLCTHMGILFREVSAKFTSQTCDCCGNAKAVSHDPSENGFDPCASMRAMKEGKNFRFKRTFICGNPACPMCQVSVNADSNAASVICHMVRNGKSDYFKDKRAKFKAPKVQKETKKSSKSKKDK

SEQ ID NO: 25 - protein, 428 aa, synthetic construct
MAKGTKNTDILYRSEKFELFWNRRPVCAPTAEELALLTITSENLRTVWNEAWRARMDAYENFFKPIYEKIGTAKKLQDEALVKGLYGELRDAFKQHGVSLYDQINALTPRRKADPAFASIPRNWQEETLDCLDASFKSFFALRKNGDADAKQPFARETPGFFCKIPGRYGFSFDGENITISFAGLGQRIVCQVPDHQRERFGNSLRLKKFEIYRDERDLSKPGSFWISVAYEIPKPPEKPVTPDNTVYLALGASYLGMVCPKGEFYFRLPRPDFHWKPLVDQVQERLKNVTKGSRKWKKRISARWRMFDILGKQQKQGQYELIQEELLSQGVHFVITDLVVRSKTGALADASKPERGGAPTGANWSAQNTGWIANLVAKLAEKAKEHGGIVVKREPSQLSPQERKMHPGERKIIIARRMREAFLADQQ

SEQ ID NO: 26 - protein, 543 aa, synthetic construct
METQYYKVAAYPMKIRLYPTKEQAKTIDSWLLGLQKAYNMTLYALKEGVPELRQKSKDGSTEFPNWKYIGSKAWLDSLRERSSYVASVPGGCLSSSVGGALGADIKKAWESQGKLPVDAWFKATDAKGHSIIRWYSDSRPRKSCFFQIEANRFTRTNQSVYITLRKDFTIKARGWNDKIRFAADSTESFFERYRDGNDVFSFRISRDNCGDYYAVITLKDVYRPFNVEPERRGIGIDAGVNAMATDSDGVSYENPRIKKKNETLKAEFGRQMARRYGIKNEQFRKERKETRKYNTLHNEEIANQTVEPRCISPSKRYLKAQVKLSELERKVRRQRDLIQHTYTARIIAKANLVAIENLNVKGMMGNSNLADSLSDAAMSEFLRKLKYKAQWSGGEYHAIGTFTASTERCAQCGYVLQGTEKLTLADRIFTCPICGNTDDRDANAAKSILEIAEEEIKEGIPSADTVKKPKEKKKKTYPDKPIGKNYPDVFTHFSEELVQQHKNPFVIVNDRQEVLDDAQGYGYSDRQSAQKFWTHKMKTQQKQ

SEQ ID NO: 27 - protein, 543 aa, synthetic construct
METQYYKVAAYPMKIRLYPTKEQAKTIDSWLLGLQKAYNMTLYALKEGVPELRQKSKDGSTEFPNWKYIGSKAWLDSLRERSSYVASVPGGCLSSSVGGALGADIKKAWESQGKLPVDAWFKATDAKGHSIIRWYSDSRPRKSCFFQIEANRFTRTNQSVYITLRKDFTIKARGWNDKIRFAADSTESFFERYRDGNDVFSFRISRDNCGDYYAVITLKDVYRPFNVEPERRGIGIDAGVNAMATDSDGVSYENPRIKKKNETLKAEFGRQMARRYGIKNEQFRKERKEARKYNTLHNEEIANQTAEPRYISPSKRYLKAQVKLSELERKVRRQRDLVQHTYTARIIAKANLVAIENLNVKGMMGNSNLADSLSDAAMSEFLRKLKYKAQWSGGEYHAIGTFTASTERCAQCGYVLQGTEKLTLADRIFTCPICGNTDDRDANAAKTILEIAEEEIKEGIPSADTVKKPKEKKKKTYPDKPIGKNYPDVFTHFSEELVQQHKNPFVIVNDRQEVLDDAQGYGYSDRQSAQKFWTHKMKTQQKQ

SEQ ID NO: 28 - protein, 539 aa, synthetic construct
MEVQYNKIDVYPMKIRLYPTKEQAKTIDGWLMGLQKAYNMTLYALKEGVPELRQKSKSGDAEFPNWKYIGKREWLDELRKNSPCVANVPGGCLSSTVGGALGADMRKAWESQGKLPVDAWFRATDANGRHIVRWYSDAKPRKSCFFQIEAGKISREKQSVYITLRKDFRVKARGWNDKIRFSEESDEGFFEKYRDSKKVLSLRVSRDNCGDYFATITLKDVYRPTKVEAERKGVGIDVGVRAMATDSDGRVYENPRIKQKFEDKKTELGRQLSRRYGAKNKQFRQDCKEARSFNRLHESEINEKLVVPKTVLPSRRYNQAQLKLSKLERKMRRKRDMAQHVYSAEVVRKASLVAIENLNVKGMMADSNLADRLSDAAMSELLRKLKYKAEWSGASYHAIGTFTASTQRCAKCGYILRGENKLARDDSVFVCPVCRNVDGRDANAAKSILQVAQKEISEGVPSADLIKKLEVKKTKTYPDKPIGKAFPNVFTHFSEEYKKQKRNPFVIVDENKNVLNDAQGYGYRNRQSAQKFWAHKMNQ

SEQ ID NO: 29 - protein, 557 aa, synthetic construct
METQYYKVAAYPMKIRLYPTKEQAKTIDSWLLGLQKAYNMTLYALKEGVPEEPEEPEEPGEKKPELRRKSEDGSTEFPNWKYIGSGDWIERLERKNQSLKGIPHDAYYNAVGGMLSVDMKKAWESQGKLPVDKWFQARDEKGHLIVRWYNKGKTRSSAYLQVEARKIVQQGKSVFITLQKGFSVKARGWNEKIRFSEDLTQSFFDKYQGDKRKVGVRISRDNCGDYYAVISLKDVYRPVKVEAERRSVGVDAGTRVMATDSDGMTYENPRIKKRNEAEKAELDRQLARRFGARNEQFRAECKAARKYNKSHQEEIAEKVVELKRVLPSKRYLKAQLKLSKLERKAARQRDMVQHIHTAQIVAKANLVAIEHLNVEGMKKDSNSASSVEDAAMSGLLQKIKYKAQWSGGNYHSIGTFDPSTQRCAQCGYVLKDEEKLKRGDDTFVCPRCGNVDGRDENAAKSILIVAKERIERGLPSADTIKKPKEKKKKTYPDKPIGKNYPGVFTHFSEEFRQQYKNPFVIVNDKQEVLDDAQGYGYSDRQSAQKFWTHKMKTQQKQ

SEQ ID NO: 30 - protein, 623 aa, synthetic construct
MKTIKIKIKLTTDQVQLCDRYLEELTWLWNLTLSNQLHNHCVTWYDWAAKLSANLDKATEKLDKLKPEQQQLIKDYYRTKDKPKLSKKEQELVAKFDIFARWNSFSLDGIIPVPLRLGNSGYEGLSCQIATGGNYWKRDENINIPINTKKGIVHVKGYKLVKGDKPWQRIEIVPHKYRTFPGGKFEGRELTTLEKLDNVNGLNTLRAFQNLPDLQVSSHYIGGLLAFFKESWSAFLDPKRMNSRKPKFKKDSDKITTLSNNQCAPNRIDVNKNIVTVTGFSPIAIIDKNWVKRLNLSEVLPRTYMLTQNPSGYYINIVIAHPLHEEKTALVKKLPKVKKEFGEDSQEYEDIKSKIKFLEQQIKEASIVKGKDLSVGIDPGVQAVVSTDHGALFLPNLTRERVSIHIEELQSRLDNIELINDKKWKSLGNKTPRIKTKNETKLQEKISRLHERGANSSNAFNHKLSTRLSRTYEHIAWEDTQINNLLKQVEPKALPEGIGYAHNGASAKRGLNWIMRQRCLSDLKAKTKQKTENRGGNFHEPPANYSSQTCHCCKQKGERRSQHEFICKNSDCKLFDIPQQADTNAARNHKQNGGFELGEVKYHNVKLVYQKPKRFKKKRLTNQ

SEQ ID NO: 31 - protein, 833 aa, synthetic construct
MTKAERLRDVAKNRLVRLERRAQKIGEHLKRKPGDLQAMHHLLHQIEVEYHDVSRNLKDPDWVPKPKRKREKRNIESTDHIPPPTKGDPGVPKHYSIPKPVPLPVDRIPEDQLKMGWKSSGRSWCSPPFVEVALPPGKEHVVIDHLSKFKIDDDREVVRAWAEKEFGSIAVAKEALKVGATLSVDAGVWRGLIEQAGKSERFSDLTGEELLADASARRIAMAWHQFEWVKQTAKSITDSAPKGVSKDALASKTRAHLKSFHTAVNSFKQVTNKETGAVELVNKHTNPQFSYLSMEKPVVDADTVSEQVVDWLSLPVDERFTKDENDPKKRGRITVLQKELGAAKRSKHWRGREQTQPWAGKPHWKGTLIRKRDALLIYDCCKNGLALVLCTGGGVRVDVESLLNMDGTDLRSDRQLLTPNGTGKSAFVLPLVPKHDFHRWYAKHVENDNADAPLTKRCIHNTTQFVVIPEHKGHPPQLFIRPVLKFYDPGKEIPDTHAWGAKPQCRYLIGVDRGINSPYFAAVYDTERNAIVAIRQGRGRKDEWKNLRNELALAQREHNELRNKRGKAKQLAKAMANIRALRKKERGLNKVETVESIAELANWAEKELGACNYCFVIEELQQMNLRRNNRVKNIAAIKDALVNQMRKKGYKYKEKSGKVDGVREESPWHTSAVSPFGWWAKSEDVDKDKRFIGRRVGGHYCRDAEDGRYIRGMYKKPGGKYGRKVFTLSEDDLRTGIRRRSFGSELFWDPHRTEFRGKPFPNGVVLNADFVGAFNIAVRPVVKDGKGKGFTAKNMAEVHTEFNPTVAIECEIPLYEFTEVDGDPLGALREVVV

SEQ ID NO: 32 - protein, 857 aa, synthetic construct
MSPLERSLRKVGENRLERLRVREEKIRKHIEQHPRGKNDHQALHFLLHQIEVERNDLYRNLKDPEYVPKPAKQRRERRQINVAKPPTRPKKEKGPQPESTKYVIRPPVPGKNLPAFASKYEARDTRDDSYQDGRSWTSAPYVEVELPILGADKVIQKLMKFVQKDERSIVRDWATKTYSSIEAAREALLVGAQVSEDVSVWRGLLAETKNAQNFAALSDDQIEAAMSKEAKGADLRPRRAALLVAQRHWVDQTVKAIKESAPSGVDKDTLDRRLRAGLRGFHTAANSGKHTNPQFPYLTAEKPVVPMESVVQSVLAFLDDPDDQRYTKDKEDDKKRHRVTVLQKELGKARPRKRLELQTPKWAGRPTVKGTISKRRDAALVWDTSKEANGLCLALPIGGMPKIDVEQFIYQDGTSLLSDCQIASKTTKKGAACAVLPLKPKHDFLRWFTKHVENHNPDAPLERRCLHNTTQFVIVDPEGPRPRLFVRPVFKFYDPGKTVPNTHETWKKPDCRYLVGIDRGINYVLRAVVVDTEEKKVIADIGLPGRKHEWRMIRDEIAYHQQMRDLARNTGKHASVVAKHVRALALARKKDRALGKFATVEAVAELVKKCEQDYGSGNYCFVLEDLDMGAMNLKRNNRVKHMAVMEEALVNQMRKQGYAYDGRRGRVDGVRHEGAWYTSQVSPFGWWAKRDEVEEAWKRDKTRPIGRKVGNWYEMPEPGQDGDRPDTYRKGYWSKPKNAEGKPYGRNRFSVEPGDEKPDAERRFCWGSELFWDPNVKSFKGKEFPEGVVLDADFVGALNIALRPLVNDGQGKGFKAEDMAREHTILNPQFKIACQIPVYEFVEEDGDKWAALRRIML

SEQ ID NO: 33 - DNA, 1479 bp, synthetic construct
  source 1..1479: mol_type = other DNA
atggatgaacagaaggaggccgtaatccccaaggtcgcgcggttccggattctcaagccggtcccgcccaccacgtggcaggagcttggtgagctgctgcgggcggtgcgttaccgcgtatttcgcttggcaaacctggcggtgagcgagaaatacctgcagttccacctgtggcgaacgggccggacggaaagtctggacgtgcgcaccgtgaaccaactgaatcgcgaccttcgcaaggtgctggaggaggagaaggaggacgaggaggcggaggagaagaagaaccagcacgacgtcgacccggcgcgactttccaaaacaggcgcattgcccgacacggtcgtcgcggcattgagccagtacaggatccggcccctgacgacgggatccaagtggagccaggtaatacggggacaaacggccttgccgacgttcaggctgggcatgccgatccccatccgatgtgacaagccgagccatcggcgtctggagcggatgcaggatgggtcggtacagttggacctgatggtcacacgtaagccctatccccgtgtgatgttgggcacacgcaacgtggggggtgggcaggcggcggtgttggagcgtctgctcgataatccagtgcaggacccctcggggtatcgtcagcggtgttttgaggtcaagcaggatgtgcaaacgggcaagtggtggctgtatgtgacatactgctttcccgccgaggctaccgcacggagtcgcgacaccgtggtgggtgtggacgtgggcgtgtcggttcctctgtacgccgcgctaagccacggacatgcgcgactggggcatcagcacttcgggcccttgggcaaacaaatccgaaacctgcaaaatcaggtcgttgcgcggcgacggtcgatccagcgggccgggcggcggggggtcgtggacaagacggcccgcgccggtcatggggtcagacgcatgctgggcgggacggagaagctgcgcggtcggatcgaccgggcgtatacgacgttgaaccatcagctcagcgccgccgtggtccgcttcgcgcggaatcacggtgccggcgcgatccaggtggaggatttgtcgggcctgcaagacactctgcgcggaacgtttctcggcggacgctggcgctatgatcagttgcagcgattcatcgaatacaaggccaaggaagccgggatcgagtatcacaaggtgaatgcagcgttcacgtcgcggcggtgcagtgaatgcggggtcatccatgaggggtttacccggacgtttcgcgaccaacacgggacgcagggccggtcggcgcggtttgaatgtccggcgtgtggctacaaagcagacgcggattataacgcggcgcgaaatctatcggtggttgacatcgaggaacgcatacgggtacaatgcgccgaacaggggttgaaggccccaacgtcggccggtgaggtggacaccgagccggaggatctttga

SEQ ID NO: 34 - DNA, 1494 bp, synthetic construct
  source 1..1494: mol_type = other DNA
atgggcgaatcggtaaaagcaataaaattaaagatactggatatgtttttagaccccgaatgcacaaagcaggatgataactggcgcaaagatttgtctactatgtccagattctgcgctgaagcggggaatatgtgtctgcgcgacctgtataattacttttcaatgcccaaggaagaccgtatttcctcaaaagacttatataacgctatgtatcataaaactaaacttctccatcctgaattaccaggtaaggtagcgaaccaaatagtaaaccacgctaaagatgtttggaaacgcaacgctaaactcatttatcggaaccaaatctcaatgcctacatataagataacaacagcaccaatccggctgcaaaataacatttataaattaataaaaaataagaacaaatacataatagacgtacagttatactccaaggaatactccaaggatagtggtaaaggcactcataggtattttctggtagcagttagagactcatcaacccgtatgatattcgaccgtattatgagtaaggaccatattgacagtagtaaatcatacacgcaaggacaactccaaatcaagaaagaccaccaggggaaatggtattgcatcataccctatacattccctacacatgagacagtcctcgaccctgataaagtcatgggagtagaccttggcgttgcaaaagctgtttactgggcgtttaatagttcttataaaagaggctgtatcgacggtggggaaatagaacatttccgcaaaatgatacgagctcgcagggtgtccatccaaaatcaaatcaaacattcaggggacgcccgtaaaggacatgggcgcaaaagggcgttaaaacccatagaaacattgagcgagaaggaaaagaattttagggatacaataaaccaccgctatgcaaatcgaattgtagaagctgctattaagcaaggctgtgggacaatccaaatcgaaaaccttgaaggtatagctgacacaacaggcagtaaatttctcaagaactggccttattacgacctgcagacaaaaattgttaataaagccaaggaacatggcattaccgttgttgcaataaacccccaatatacatcccaaaggtgttcgatgtgcgggtatattgaaaaaaccaaccgttcatcacaggcagtatttgaatgtaaacaatgcggttacggcagtaggactatatgtattaactgcaggcacgtccaagtatccggggatgtttgtgaggaatgtggcggcatagtaaaaaaagaaaacgtaaacgcagactacaatgcggcaaaaaacatatccacaccgtacatcgaccagataataatggagaagtgtttagaactaggtattccttaccgcagtataacctgtaaagaatgtggtcacatacaggcttcaggaaatacctgcgaggtttgcggaagtactaatattttgaaaccaaagaaaattagaaaagcaaaataa

SEQ ID NO: 35 - DNA, 4101 bp, synthetic construct
  source 1..4101: mol_type = other DNA
atgaaccgtatttaccaaggtcggattaccggcattcttgattccaaggaggacgagcggggccaccctcccccacccgatccgaaacacaatccattctggcggcatcacgaggtgtaccaggcggcggtgaactactactatgtggcctttgcggccttggggcgcgccgcatcggacgggatgttgcgcgacttggtgcaacgggtcggcgaatcctgggagaccgatccggttcacttccgacccgagtacgggctcagagcttcgctgagacagtttctgcctctttccgacaccagcacgctcgaagaggccttcgaacgaattctcgaaaaagaccacgcttctcccgacacccttctcaaagcggccatggcggtggcgaaagatctggggggagagtccagcatccaaatgaaaggccgggaatatttgccccgtctctgtgtgcccggatacaaggggaagtttccccgcgaaaaaaacagtctggaaaaggaacgaatgaaggtgttgctgccagtgtggctccacgccgaggaccccgcctcgcaacccaccttgaagaaaatccgcttccaccacttcgccaaccccacgggaaccagactggaagtcgctgaatccaaagacctcctcggcgtcgcgatcgataccctcttgcgggaaaacaaaatcaccgcggaggaagcgggcacgctccatgacgaaatcgacgccctgcccgacaccttcaccctgcccgcctacgccgggggaagcgtcaacaaagacgcgctgaaactccggttccacgcttatttgattaccgaacatttttggaaaaacggccgaggcctcgaattgctccgctccacctatcctccacccaaaggaaaaccaaaaaaggtcggactttccgaggaggaggaagccctgctggtggacggagacgatccggtcaaggtggcccggggagatcgagggtacgtcttccgcgcgttcaccagccttcccgcctggggcggacggagcgcaaaggacatcgcctggaaggaattcgacatcgccgccttcaaagaggcgctgaccacctacaatcaattcaaagataaaaccgaggaacgcgcggcgcggttgcgggacgttcaggcccgactcgcccgaatgaacggcaacgaaccggaaattccgctggcgaaggaggacaaggacgatattccccgactggagaacgatccacgggtggcgcttctgggagagcttttgcatgactcgggcgtcgtcgcggaaggccaaaccgtcgaccggggaatttaccaccggtccttgcggcattaccgggaccttcgatccgagtggaaccgtgttctcagccgggcctctgacggagattcggactcggacatctcaaaacggctgatttccgtcgtcaacgaaatgcaggcaaagtacgcccacaccttcggggacgtgaacttgttccggggcttgtgttccgatcccaaatactggccggtgtggaaacatcccgatgccgaaacgaaaaaacgaattgaaaaggagggctgggcggaaaacgtcgtcgaggcctaccgagactttctcgaacttcaggcggaggaggagcgctattccgaagccatccatttccgcccggcacaccccgaggaatcttcgcggtacttccggttttccgacgtcgtcaccaagaatcaaaccttgcatgaacaacccggagagcggctcaagcttccgatcgtcgagaggaacgacgagggcgtgtatacgaaatcgagcattcgcgtccgctattcggccccccgcctttatcgtgacgccgtgtgtgggaatggaaacgaaaaggggcattggttgcagcccatggtcaaagcgctctcactgcccgagcctcccgaggcggacatcacgaacagcgcgatcgtgctcaacatcaaaaccgacggggaaaacagctcggacaagcgtgtgcgggcctacgtcgatttccccgtggaactggacacctcccctctcaaggaacacctgggccatacccggaaatgggccaagcaattcaacggccaatacgaccgcggcgtcccctatccctccgccggcggcggcctctactggcccgacatgcaggggcttccctccgacccttggtacgaaaacccggcgatacaagcctccggatttcaagtgctcggagtggacctcggccaacggaccgcggaatgccatgccctgatcgaaattcggtgcgacgggaagttccctcaaaagaaggatggcacgtcccgggatatcctcgccacggtgggccatgatggaacacggacgtgggaagccgtgctgttgcgggccggaaccggacggttgcccggcgagaacgcccaccaaatggaacagggacgccgcgtacgcgaaaaaggggggcgacggggccgcaaaacctccgacgaggattatcggatcgccgtggaaatcctggaaaaaatcggcctgcgcgaaatcgcggaatcccaccgtcccttgaatgattttaaatacgcccccgaactctgggatttcgtactgcggtacctgaaatggattcgaaatcgactgggccgcctgtttttcgccgccgccgatttgcgggacgacgagctccgtgacaaggtggcgacccggttggaggagtatcccattcccggaatcgaactcccttccccgttgaaggagcggcacgaggaagccgcggagctgtgccttgcccgatacacggaactgctcgagaccttccgctccgcgctactcctgttgacggatcagatccttcctctgcgtggccgccgatggacctgggcaccccacccccagttcccggagagcacgcaccacatcctcatggaaacgccggagccttccacaaaaggccgaaaaatccgagggcaacgcgggctctctttcaaccgaattgaacaaattcaggaactccgaaagcagttccaggccctgaatcgcatggagggatgggatattcaggccccccatcgaccgggacgggacgagatccgccaaagccttcccgagtgctgccaacctctgctggaaaaactggacaacctgcgcacccaacgcgtcaaccaaacggcccacctgattctacaggaggccttgggagtcgagctggcgcccggcgaaaaaaacgcgacgggggacgaacacggcgtctatcgagtaaaacacggaagaaggcctgtggacatcatcgtgttggaggcgctgggggatttcaaaacgtctcaaaaacgcgggcggggcacgaaccgccgcctcgcctcctgggcccaccgtgggatttcggccaaattgcgtgaactggcggaacccttcggcatccccgtggtggaaacgcctccccacaacacgtcgacgttccatgccttcacgggccgccccggatacagggctcgggaaatgtccggaatcgaaatggagcgaatccagacaaagctcgaagaaaagaaaacgccaaagaccttccgggagacattgatcaaagcaggaatcgacgccatgcgggaaagcgggaccccgcccgaaaaaatgaccctgttggttccgcaacaaatgggggagctgtttcttcctctgaccgggcccgacgaacccgcgcttcctcccatccaatcggacatcaacgccgcgatcaatatcgcgttgaaaatggtctccgctccggaggcggtccatttgcggcacacggtgcgatttgaaaccccggcggggaaaccggtcactcccggcaagggaagcaaactcgaaaaagccctggccaaacggaaaaacgtgaccttcgaaatcggtgacgaattccatgcgccgcaggcgcgtatcgaccgggcgaatctgctggggacaccggcgggcacggtccacacgcccatcggctccttcgactttatgtcccgtgacgccatgagaaacctcctttcccactacaaagacgacatgtttattcaggccaacaaacgggcactcgaaaagcggggaatttcgtttcccatgacaccctccaaccaccccttctcggcgggtgatttcatggacgagcattttcccatgtaa

SEQ ID NO: 36 - DNA, 4083 bp, synthetic construct
  source 1..4083: mol_type = other DNA
atggctactcgtatttatcagggacgtattgtgcaggctcgttttgaagatgagaacctgccgcaggataaggaatctgcattagccgcattggaaagaacgaatcgtctttttcaagatgctgtcaactaccacttggtcgcgttggcagggatggcggaagatgggaaagagactctgggaagtagattcaagaaacaggtgagggcaatatgggaagatttgcctagaagtaaggttggtgcttgtacgttgcagcgctccatagcacgtactttgagtctggcagacggagttacgtttgatgatgccgtggcacatatttatgaagggtgcgataggctggatgttttgccgtatgtcgagcattacgtgattgaacaaacacagaagggggagggagctattcaacaacaaggacgagagcttcttcctaaattgtgtaattcggattttgaaggtaattttgattatagtatcaaagaacgtaaggcgaattctgggaaacagaagctgctgagggagcttaatcgcgatgatgtttctgatgaggagttatacgctttggcgcaggaaatggacttatcgtgggttgtgaaaacaatacctaatgaaaatcatgatgagcccttgtattatgatcctgaagaacaggaggaaaaggtaataacttctattcataaattattgtcatctttagataatgggaacttgaaaatagcaactctccccaagtatgtgcaaattgacggttttcgtgagcaaatgcggcaggatcttcttaatcgaatgcctttaaaaggtcttcgcttggctaagagtagtcggggaatttccgtagatgtggaacaggcgggtatatacttcatgttttatcctggccgcatttctgctgcgcatctggcgtctaaacttggaaaagagaagcctaaaaaaacggatgaagtaagagagtatgattgcttcagcctggaaaatgacccgctcatattggctcgcggaaaacgaggctacgtttacaaaggtttttcggcattgcctaactgggaatcatcggataaccgcatgtattccaaagaatgggatattcttgcgtttaaggaagctttgaaaacgctgcatggttttgagcttaaaacgaaagaacgtgatgcagagcgggcgaagtatgttgcgcagttagaatatgttgagagaggtaaaaaaacgaaggattatgtggaacctgatgaggaaaagactgtggctgtattagggggtgattatcggtttgagcttctcaaaaaacttgtagcggaaatttcgcctgaccctgttactgaataccacatttcctcacgaacactgaatgattatgaggaagtgagggctctgtggttgaaaagggagctcacgggggactgcagcagccacgatttatccaagattgttcgtgaatatcaggctaaaagcaaaagatttggctcccaagtgctgtttgcagcgctgtgtcaggatgagtatcggcctatctggcatgattatcatgcattgaaagatgagaaacttcctaggtccaagaatatactgcgtgatttttccaactggcaatacttgtgcgggcaggttgagaaatacagccgtgatgtcagggtgactgctgcggatgtggtagcatctcctcgccagatgatttattccgatctttccaattttggaaatggaaaggggtgtgagtatattcccggttgtgagggtggtttgcgtatgcaggtggtcgttcggaatgctaaaggacattgggaaacggactcgattcgtgttactttctcagctccaagatttttgagggatgagatggggcaggacgctggcaaatggatggttcctaaaaagggagaaaatacgaccctgccgtggttgcaaccgatgatgaaggctttgggggatgatattgcacctgttcgcctggaacgtacgcctgccattggtttgcaggtttgggggcagggctcggaggcttgttattacctgaacttccccgtgtcactggatgtgtctgctcttcagaagtctcttggcaaggctgctcgttgggccggtcaattcctcggaggcagggatgagaaacttcatttgcactggcctgctacctacaaaggtaagaatcgcccttggtgggaacaggaaaaggagtttacagtgttgggcatagatttaggtcttcgtagctcggttgcctggtcactgttgcgtgtctcaacctgctctacttcttctaactccagaggtgatgagctaataggtcgcttgataggggatagctcaaacgctaaatggtatggctatgtgattaagcaaggtttatctcgccttccgggggagcaacctcgtcgagttggcgataaaaagattccagccgtaagtcttgcctcccctgaagatagaagaattgccaagttgattattgaagctgctggtgctcagtttgatgagaatgaatctgaagatgtgcttcgtctgggcaatagaacgctcaaaagctttaagagtttgacaagtcgactgaaaacatacttgtctttcttgtccgggcttaaggatcctggtagaaaacctgctgtgttaaagcgcatggcggattattttgcgtacgcagagataatacccggagtctgtgttcttctggaagcgcaccaggaagatgaggtttatgagagggttctggatgcagctgaagagctgcgcagtatgttgcctaagtatgcagaacgagtaacaagccttattttgcctcgcaaacacgggtcttggaaatgggagccggagcgacgcaagggctggcagggctccggcgtgatgcgcttaacagaagaggacggtataccgcatcgaccagttttccatcgcggtggtttgtctgttgcccgtttgactcagcttgaaacgctcaggcagctgttgcagtccatggggaaagtgttatcatttgttcctggtgaatctgttacatttggacgcaggttaaaagatgaaaaagtcattgatccatgtcctgagattttggaaaaaatcgagaatatgcgcgagcaacgcgtcaatttgattgctcacgacatcgttgctcaggcattgggcgttcgtttgaaatcctctcgtcctcacaagaattcaggtgggctggatgttattcatggcgaatatgagcgtattcacgggcgagagcctgtagattttgtcgtaatggagaatctcagccgttatctgacttctctggaccgggctccgcttgagaactcagggttgatgcgttgggctcaccggcagattgtggcaaaggtgacgcagctccttgaggaaatattcggcattccggttgtctttacccatgctcattacacttccaagtttgattcaatgacctcggaaccaggtttccgtcccgtgatgttgaagccggagtatctgaagtggttgcaacgaaatggaaaaggaaacgaatgtaaggcggcagctgtatatcaagccatatgggatgaagttgtgaatgctagcaagacgaaaaaggtgactttggtattgccgcatttgacaaaaggaggagagctggcaaatggaggtgagctgttcttatctcagaaaggggggaagtttaccttgcgcaatgctgatatgaatgcggctacgaatgtcgcttggcgaggattggctgccccggaatcgctgcacttgctgcaccgggtccgtatggagatgaagaaggctggttttgtgccggtgtgcgacaatgctcgcgagaaaagcttgaagacagggtggagtttgaccaagttgaaatccattcagcccgaaggaaataagatatccgcatttgctgtttcttccgagtggaaagatgagtactttgctgcctatggtaatgcagagacgagggcttatctggcttacgggaaaacgctatgggggataatgaagagaaaacagtggcagatgtgccatttgttcaatattcagcagttgaaaaatgcaggcatagacgcgcgcttggtggaacaattgctgcatactgatgaaaacgacacatcggatgatattcctacatga

SEQ ID NO: 37 - DNA, 1641 bp, synthetic construct
  source 1..1641: mol_type = other DNA
atgttcaaaaccgcgcgcttcaaggttcacaatccgtcacggcacaagagcacgatgctctggtacgcgatgactcgctatcacaacaccttgaaggatgtcctcgaaaagacactggccatcccaaacctgcttgaacaagtctccgaactcgacaaaaaagaaaaacttcggcccaacaaattcaaactcagcaaattgctttacacgatcgcaccacaaaattgggaacttgcccctctgcgtgactatctcatcggcgatgcctctgcgatgctgatgagccatctgaacaagacctacaagggcgcgaacgagtctaacccacccactgtctccagcttaaatgcaatgactgacgctgagtttcacaaggcctacagcgactttacggacccggaggcaaagctcgccatcaaaccgcagcatcaggaaaagatcgacaaggctgccgagcgaggcgagacaaacgtcgcaaatcgtctgagcaaaatctacgcgaattgggccgcctcccgagctgcgggtcaggtgctgcgtaaactagaaggtgaactgcctcaccccatcgaattcacacgtaacgagattgggcggggttgcttgcttgcctattgcgacggtaactactatctgctcgtccgtctattcgcgcaagatcatcgctattgtgaaaagcgcgttctcaaagaaggattcatcaattgcaaaacaaaggaacacattgaaggcaaaaaatatcctggaatgattctccctctggaactgggccgcgaattccatgagcgggaatatctcacgcatggttccatccaaagcgccaagctgattgtgaagcggcgggaaaagccaaactcacagccgaaagccctggactcaaaacccgccccttttaacgcggaagactacgatttttatgttcacgcggcctttgagtttcagccgacgcagatcgaaacggagacattccttggcatcgaccgcggcgctgccaagatcggcgctgccaccttgattgaccgacagggaaagccccttgaaaccgatctcgatcttagcggcgcagcctttgcggcggagatgcggcgctttgaacagcagatcaagcgaattcagaaacagggaaaacagaagtcacgcaagttttcactacgcggcaaacgagcagatatcattctgggagagtacgccaaccggattgttgctattgccaaagagaatcgctcacagattgtcatcgaggccatcaagggcgtgacaatgggcagatttttgaaacaaagccaattcaccaagctgaagcagatgctcacctacaaggcggagcgagagggactgccagctcctatcgaagttcctgcggcgttcacttcacagacctgcgccagatgcggccataaagatccggccaatcgtccaaagaaagatgcggctggaaaagcgattcaagatgtgtttttatgcacggcttgcggtcatcatgccaacgccgattccaatgcgagcctgattattgcgctgcgtgggctgcatcagattgaaaatggtggtaaattcaagaagttcgatctctttcaacaatggttgaaggaaattatcggccgggacggctcctttgccccagggcaggtgagcccgtag

SEQ ID NO: 38 - DNA, 1581 bp, synthetic construct
  source 1..1581: mol_type = other DNA
atgtttaagacggcgcggttcaagattcacaacccgtcacgccacaagcaggctgtgctccgctacgccctctcccactaccacctcacgctgaagaacgtgcttgaggccgccctcgccgatcctgagctgcaggcgcgggtgacggtggtgggcaagaatggtaagccgcgaaccgataaggctgctctgagccgatttctctacgctctggccccaaaaggctggccgctggcgcccttgcgcgactacctgataggcgacgcaagcgctatgctgctcagccactacgagaaggacctgaagggcaagaacgaatccaatcccccgacgctcggcggactcgaaggtttgacggaggagcgccgccgggaggcgttccatgacttcgtcatgaccgaagagtttcctcttcgtgcggaccgcgccgccgagattgaaaaggcgcggtctctcggccaagtgcaccttgccaagcgcctggggaacatttatgcgtcgcgggcgagcgcaaatgccatgcgggatctgctgcggagcctggacgcgccgattccccggccgatcgagttcaccagatgcgagtttggacgcggtttcctgctggcgcgaaagggtgacaagttctatctcctgctgcggctcttctcgaagggaaactcctactggcaacaggtcatgttggacgaggggttcgtcaactggaaaacgaaggagaccattggcggccggaaatatccgggcgtggtccttccgttggagttcggacgcgacttccacgaaagcgagtacctggagcatgggtcgccgcagagcgcgaagctgattttgaaacggagcgatgagggtctggaggagttctgtgctcacatcgctttcgaattcacccctgaacccgtcacccccgagacgtttctcggtatcgatcgcggagccgctatgatcggggcggcaaccgtgatcgatcaggctggtggtttggttacgcggcgcttggacctggagggaacggcgttcaaccgcgaattgaagaggtttgaatttcgtattgcaaaggcacaacggaaggcacagcgcaggcctcgactgttccgggtgcggcgccgttgggccgcaatcgtcatcggcgaatacgctaacagggttgttgcagaggccgtcaagcatcgctcgcagattgtactggagaagatagacgctcgctccatggcccggttcctgagccacagccagttccggaagctgcacgatgccattacttataaggccgagcgcatgggcttaccaaggcccatcgaagtcccggcggcatacacgtcgcagacttgtgcccggtgcggccaccgggatgcggcgaaccggccgaagaaagacagcgagggccgcccactacaggccgtgttccggtgtgtgcggtgcgggtgcgaagccaatgccgacgggaacgccagcgaggttatcgctctgaggggtttgcatcaggcgctcaatggtggtaaatttcagaagttcccagccttccaggaatggttggtcgggatcagacgccgggttggtgagccagctttaatcggccggtga

SEQ ID NO: 39 - DNA, 1785 bp, synthetic construct
  source 1..1785: mol_type = other DNA
atgttcaagaccgcccgcttcaaggttcacaacccgtcacggcataagagcacgatgctctggtacgccatgactcgctatcacgagacgctgaaggccgttctcgaagagacgctggcgattcccgatctgctggaacaagtctccgagttggacaagaaggaaaagctccggccaaacaagttccggctaagcaaactgctctacacaattgtgcccaaaagctgggaacttgctccgctgcgcgattatctcataggcgatacatccgcgatgcttatgagccacctcagcaaggcgtacaagggagagaacgagtctaatcctccgacggtctcaagcttggccgcaatgaccgacgaggagtttcgcaaggcatacagcgagtttacagacccggaggcgcagcttgccgtcaagccgcagcatcaggaaaagatcgacaaggcgagcgaacgaggcgagacccgcgtcgccaaacgcctgagcaaaatctacgccaattgggccgtctctcgcgccgcagggcaggtgctacgcaggctggaaggcgcgctgccccatcccattgagttcacccgcaacgagttcgggcggggctgcctacttgctttttgcgacggaaactactatctgctcgtccggctcttcgcccaaggccaccgttattgcgaaaagcgggtactgaaagacggcttcatcgactgcaagacaaaggagcggctcgacggcaagagatatcccggcctgattctgccgcttgagctgggccgcgagttccatgagcgggaatacctgacatacggggccatccaaagcgccaaactggtggtgaagcggcgggaaaagcctgcctcagggtcaaaacaggctgcggagaaacccgctccggagggaggcggggggttcaacccccgcataaagccgacagaaccaaagctggctttagccacggaggaacgctttcccccaattccacccgaaatcccaagtttttccgcggcctctaaagcgctggcccttcagcccatccccttcaacgccggagactacgacttttatctccatgccgccttcgagttcaatcctccaaaggttgagactgtaaccttccttggcatcgaccgaggcgcggccaagctgggagcggcaacgctgatcgaccggatgggcaaggccctcgaaaccaatctcgacctggacggcgacgcgttcaaggcggagatgagacaacatgaagagcaaatcattcggttgcaaaaactcggaaagcaacgctcccgcaagttctctctgcgcggcaagcgcgccgacatgatcctgggcgagtatgccaaccgcatcgtggccatcgccaaacaaaaccgctcccagatcgtcatcgaggccatcaagggcacgacgatgaatctcttcctgaagcaaagccagttcaccaagctgaagcagatgctcacctacaaagccgagcgggagggtcttcctgccccggtcgagattcctgcggcgcgaacctcccagacctgcgcgaagtgtggacattgggatcgcgcgaaccgacccaagaaggacgcggcgggaaaggcgattcaggacgtttttttgtgcaccgcttgcggccaccgcgccaatgcggattccaatgccagcctgattattgccctgcgggggctgcatcagatggaaaatggtggtaagttcaagaagttcgatctctttcaacagtggttgaaggagcttatcggccgggacggttcctctgccttcgggcaagggaaccagtag

SEQ ID NO: 40 - DNA, 1662 bp, synthetic construct
  source 1..1662: mol_type = other DNA
atgttcaagaccgcgcgcttcaaggttcacaacccgtcaagacacaagagcacgatgctctggtacgccatgacccgctatcacgagacgttgaaggccgtactcgaagagacgctggcgattcccgatctgctggaacaagtctccgagttggacaagaaggaaaaacgtaggcccaacaaattcaagttaagcaaactgctctacacaattgtgcccaaaagctgggaactggctccgctgcgcgactatctcataggcgacgcatccgcgatgctcatgagccacctcagcaaggcgtacaagggagagaacgagtctaatccgccgacggtctcaagcttggccgcaatgaccgacgaggagtttcgcaaggcatacagcgagttttcagacccggaggcgatactcgccgtcaagccgcagcatcaggaaaagatcgacaaggcgagcgaacgaggggagacccgcgtcgccaagcgtctgaccaaaatttacgccaactgggccgtctcacgggccgcagggcaggtgctgcgcaagctggagggcgcgctgccccatcccattgagttcacccgcaacgagttcgggcggggctgcctgcttgctttttgcgacggaaactactatctgcttgtccggcttttcgcccaaggccaccgttattgcaaaaagctggttctcaaagacggctttatcgattgcaagacgaaggcgccacttggcgggaagaaataccccggcctgattctgccgctggaattgggccgcgaattccatgagcgggagtatctgacttacggttccatccaaagcgccaagctggtggtgaagcggcgggaaaagcctgcatcaagggctaaggcccgcgccgctggcgatgcattgaagggcggacagcccgtccccttaaacgccgaagattacgacttttatctccatgcggccttcgagtttaatcctgcgaaggtcgagacggagaccttcctcggcatcgaccgtggcgcggccaagctgggcgcggcaacgctgatcgaccggatgggcaaggccctcgaaaccaatctcgacctggatggcgacgcgttcaagttggagatgagacaccatgaagctcaaatcattcggttgcaaaaactcggaaagcagcggtcgcgcaggttttcgctgcgcggcaaacgcggggagatcattctgggcgaatacgccaaccggatagtgtcgatcgccaaacagaaccgctcccagatcgtgatcgaggccattcgtggtgtgaccatgggccggttcctgaagcaaagccagttcgccaagctcaagcagatgctcacctataaggcagagcgggagggcctgccaaccccggtcgaggttcctgcggcctatacctcgcaaacttgcgcaaaatgcggtcacaaggaccctgccaaccgtcccaagaaagacgcggcgggaaaagcgattcaagacgtgttcaagtgcgtagcttgcggccacaccgccaatgcggactccaacgccagcgtgattattgcgctgcgggggttgcatcaggtagaaaatggcggtaagttcaagaagttcgatctctttcagcaatggttgaaggagcttatcggccgggacggttctgttgccctcgggcaggggaaccagtag

SEQ ID NO: 41 - DNA, 1635 bp, synthetic construct
  source 1..1635: mol_type = other DNA
atgttcaagactgcgcgtttcaaggtccacaatccgtcacggcacaagagcacaatgctctggtacgcgatgacccgctatcacgagaccttgaaggacgtgctcgaaaagacactggcgattcccgatctgctagagcaagtctccgaactggataaaaaagaaaaactgaggcctaacaaatatactcttgctaaacttgtccgcacgatagtccccaagaacacgggactcgcctcgactgtgcgcgactatctcatcggcgacgcatccgcaatgctgatgagccacctaaacaaagtctataagggcgcgaacgaatcaaatccgccgacggtctctagcctcgctgcaatgacagatgaggagtttcgtagggcatacagcaattttacggacccagaggccaagctcatcatcaaaccgcagcatcaagaaaagattgacaaggcaatggaacaaggcgagacacgcgtcgccgaacggttgagcaaaatctatgccaattgggccatctcacgcgctgccggacaagtgttgagaaagttagaaggcactctgccccatcctatagaattcacccacaccgaatttaagcgcggttgtttactcgcattctgcgacgggaaatattatgcgcttatacggcttttcgccgataagcatcgttacaaacagaagaattttctcaattccggctttatcgattgcaagacaaaagagtcgcttggggggaaggaatatccaggactgattctgccgcttgaactgggccgcgagtttcatgagcgtgagtatctgacgcacggatccatccaaagcgccaagttgttggtgaaacggcgtgtatattcaaattcacagaccaaagccccggactccaagcccaccaccttcaacgccgaagactatgatttttacatccacgctgccttcgagtttcaacctacaaaagtcgagacagaaacctttcttggcatcgaccggggcgctgcgaagatcggcgcggcaaccctgataaaccttcaaggcaagctccttgaaaccaatctcgacttggaaggttctgcatttgcttcagagatgcggcgctttgatgaacaaatcaaaaggattcagaaaacagggaaacagcgttcgcggaagttctcaatacgcggaaagcgtgcagacatcattctgggtgaatacgcaaatcgcattgtggccatcgccaaacaataccgttcccagattgtcatagaggccattcgtggcgtgacgatgggccggtttctgaagcaaagccagttcaccaaattgaagcagatgctcacctacaaggcagagcgcgagggtttgccagcccctgttgaagttcctgctgcttatacatcgcagacctgcgcacgatgcggacataaggacgctgctaatcgccccaagaaagatgcggcaggaaaagcgattcaagacgtattcctgtgtatggcttgcggtcacaaagccaatgcggattccaacgctagcctgattattgctttgcggggtatgcatcaaaaagaaaatggtggtaagttatataagaagttcgatctctttcaacagtggttgaaggagcttatcggccgggacggttccgctgccccagggtag

SEQ ID NO: 42 - DNA, 1686 bp, synthetic construct
  source 1..1686: mol_type = other DNA
atgttcaaaacggcgcgtttcaaggttcacaatccgtcacggcacaagagcactatgctctggtatgccatgacccgctatcacgagactttgaaggacgtactcgaaaagacactggcgattccagatctgctagaacaaatctcagaactggataagaaagaaaaactacgtcctaaccagtacaagctcaggatgctcctccgaaaaattcttcctagaggttgggagcttgcaccgctacgcggatatcttgaacttgatgcgtcagcgatgctgatgagtcacttcaataaagcatacaagggggcaaacggatccaatccgccgacagtttccagtcttgaccctatgaccgagaaagaatatcacgaggcttacaaagaatttacaaacccggaagctagtctttccattaagccacagcaacaggaaaagattaacgcggcaagtgaacgaggtgaaactcgcgtcgccaaacgcctgagcaaaatctacgccaattgggccatctcccgcgccgcaggtcttgttctcagaaagttagaaggcactctaccacatcccatcgaatttaccaacaacgagtttgggcggggctgcctactcgcctattgcgacggaaactactatctgctcgtccgacttttcgcacaaggcaaccattattgtaagaaacttgtactgaaagatggcttcattgattgcaagacgaaagagctacttgctggaaagaaatatccggggctgattctaccgctagaactgggacgcgaattccatgagcaggaatatcttacctacggttctatccagagcgccaagctgattgtgaaacggcgagagaagcctacctcagcggctaaagcccgcattcataacacaggattggcggcacgattgaagtcgtgccctgatacaaagcaaacagcctccttcaacgcggaagactacgatttttatgttcatgcggccttcgagtttcaaccgacacagatcgagacagaaaccttccttggcattgatcgcggcgcggcgaagataggcgcggcaactttgattgaccgtgaaggcaagccgattgaaactaatctcgaccttgaaggctcggcatttgcttctgaaatgcggcgctatgaagaacaaatcaagcggatacagaagacaggcaagcaacgatctcgcaagttttcactgcgcggaaagcgcgcggacatcattctgggtgaatacgcgaatcggattgtggctatcgccaaggaataccgctcccagattgtgattgaggccattcgaggcgtgacgatgggacggtttctgaagcaaagccagttcgccaagctgaaacaaatgctcacctacaaagcagagcgcgagggattacctgcgccggtcgaagttccggcggcgcgcacctcgcaaacctgcgcacgatgcggacattgggacagagccaatcgacccaagaaggacgcggcgggaaaagcgattcaggacgtgtttctgtgcgtggcctgcggtcacaaggccaatgcggattccaatgcgagcctgattattgccttgcgagggatgcatcaaaaagaaaatggtggtaagttcaagaagttcgatctctttcaactgtggttgaaggagcttatcggccgggacggttccattgcctcagggcaggggaaccagtag

SEQ ID NO: 43 - DNA, 1665 bp, synthetic construct
  source 1..1665: mol_type = other DNA
atgttcaaaaccgcgcgcttcaaagttcacaatccgtcccgacacaagagcacaatgctctggtacgccatgacccactatcacgatgcgttgaaggccgtgctcgaaaagacgctggctgttcccgatctgctggagcaaatctcagaacttgacaaaaaagaaaaacttcgccctaaccagtacaagctcaggaagctgcttcgtacgattcttccgaggggttgggaacttgcacctttgacacgctatctcgaattggatgcagctgcgatgctgatgagccatttcaataagtcgtacaagggagcgaacgagtccaatccgcccacagtctccagcctggatgcgatgacagacgcggagtttcgcaaggcatacagcgagttcacagacccggaggcaaagctcaccgtcaagccgcagcatcaggagaagatcgacaaagcaaaggaacgaggcgaaactcgcgtcgccaaacgtctgagcaaaatctacgccagctgggccgtctcccgcgccgcaggacaagttctgaaaaagttagaaggcgctctgccccaccccatcgaattcaccaacaatgagttcgggcaaggctgcttactcgcctattgcgatggaaactactatctgctcgtcaggctcttcgccaaaaaacacaggtacaagcagaataatgtactcaaggacggcttcatcagctgtaaaacgaaagagcctcttggcgggaagaaataccccggcctgattctgcctcttgaattgggtcgagaattccatgaacaggagtacctgacttacggctccatccaaagcgccaagctggtggtgaagcggcgggaaaagcctgcatcgagggctaaggcccgcacctcttctcgcatctcaacggacaagcagaagcccatccccttcaatgctgaagactacgatttttacgttcatgcggccttcgagtttaacccaccgaaggttgaaactatgactttcctcggtatcgaccgcggagctgcaaaactaggagcagcgacgctgatcgaccagcacggcaagcgtctggaaaccaatctcgatctggacggctcagcattcgctgcggagatgcggcgctacgaacagcagatcaagcgaattcagcaacagggaaaacagaagtcgcgcaagttttccctgcgcggcaaacgagcagaaatcattctgggagagtacgccaaccggattgttgctattgccaaagagaatcgctcacaaattgtcatcgaagccatcaagggcgtgacgatgggccgattcctcaagcaaagccagttcaccaaattgaagcagatgcttacctacaaggcggagcgagagggattgccagcccctatcgaagttcctgcggcgttcacttcacagacctgcgccagatgcagccataaagatccagccaatcgtcccaagaaagatgcggctggaaagtcgattcaggatgtgtttttgtgcacggcttgcggtcatcacgccaacgcagattccaatgccagtctgattattgcattgcgggggatgcatcagattgaaaatggtggtaaattcaagaagttcgatatctttcaacagtggttgaaggagcttatcggccgggacggttcctctgccccagggcagttgaacccgtag

SEQ ID NO: 44 - DNA, 3501 bp, synthetic construct
  source 1..3501: mol_type = other DNA
atgaaagatatcaaacattcacttataagtggacaaaaagctcaaaagttcagaaagcatttggcaatgtcatctgccagcgcatcagtcaagaaaaataaaacatcgattttgacatataaattcagacttgatttaaatgaaaatatgtcagaaatacaagacagaatacctaagttttcagactacataaaattatataataaaattgaaggtgtagagcctggtacattaacacattatttatgcacctttgtcctggctggatttagacttttttccaatgcaaaatcagcatttgaatttataaaatcacaaaataacccatgtttagagcacttatcctcacataaacttttaaaatccagcgctgttgcttttgatcttacagcaaatcttgctatctcagagcctggctatgagccatatttggcaattgccagaattcttgagagatacaccgatccggacaagaaaattaacagctttgtaaaagacaactttacaacatataacaacaatgctctatcctggcttttgggtaaaggtcataaattttttaaagaatctacagcgcaagaaatagctttgtattatggcataccagattataagtttgactgtgccaaggccattaaaaatgcagctgacaaattagagttcaacagttcactattttcaaatgacatgcgtctttctcaattcagatcatgctttggaggacatattgacagctgggcaactaactatataaaaaggcttttggagcttgaaaaaatcatagcaaacattagctatgaaatcaaaatacctaaagcttttatctcctcttccaatgactttttaacacattgtaatcttaaccgcgacgatattgaagaattaatttctaatataaaaagcagttcaaccattactgatgtcaaagatgctcttagcacattactgggacacaaacaaggcgcatcatctgccgatataaaggcaataagagattactcagagctaatcaacagactttgtgcttataaagagcagatatttaatacaatagatcaggccgctgaagacaaaaattcattgtggcatgacataagacgccaaacaaaagatgaattgcagacttgggagaaattagaaaaactgccaaaactaaatgatttaagtggcggagttccgcaggctgagaatgaattaaacgcaaaactaatgcagcttaaacttgtaacagaggcacagaacaatcattttgcaaagattatgcagtgggtgcatagcaatattaaagacttttctccctttaatcatatagtgcagactgagcaggaaaaattagataacagaccaaaggaaaatacaactgcctgcgatcttgccgtgagaatgttcctgcataaagtcggacgtatagcccgcgaagataacaacaatctatgtaaagagttgcagcaatggtttttagataataaagtttttgataataagactgattttaataaatactttcataataagcttggctctatatacatatccccttacagcactcagaaaaatgcaggatataaaataaataaagaagtgttaaactttggtgaaaaaattgtcttgcttttcactgacaaattacaagagattaataagaggtatgaaggtaattctattgctgaaaaatcagagttaaattcacttttaaaattaaactatttttactataacttctttatatctggcataaataaagcagttcccgtttctattgtcaaaccgctactgccagatgatatgctagagcaatctctatctgcaacacacaaaataagattaaaaagcaatgaagtggatccttcttccctaagctccatttttaatatttataaatcattaataagtggttgctatacagtattaaatcgtgaaactttcttcttaagaacaaagttttcctggattgaaaacttcaccctgttctatgttccaaaggcagatgcctcgtggataatgcctaagagatacctaaaaaatacacgctggcagcagtatatagaagaggaggttttagtttttgaaaatgataaatataaggtagatataactcagacatttaataatatatgctctgcgcctgctgactatgctgagcttcttgttcaattacctcatgactggttttatcaattgccatatgaatgtgccaaggaagacaattatgtacaggccttagcaatatgtaaagataaaggattccctaagcagagcagattaaatacgcacatatctggcagacttataggtccatcatcatttaagtcaaaactagactctgtacttatatataacggcgatgtcactatttctgatatgacgctgcttgttgagcagagagtcagtcagcagctaaagcccgatgagagtcttgagcttaaaaagtatgatcctgaatttacacttgcaatccctattaatgatgcaagatctcagagcaccaattacagctttaaacacataattgcaattgatcaaggagagataggaccttcatatgcagtgtttaatttaagcgatgccggcaatgccaatgcagagcctattgcaactggctcaataagaataccttcaatcaggcgccttataaaatcagtttcttcttttagaaagaaaaagagtacaacgcagaaatttaatcagcgctttgattctacaatgtttaacatcagagaaaatgtcacaggcgatatctgcagtgtcattgtaggtcttatgcaaaaatataatgcttttccagtgctggaacgagaagtttctaaccttgagagcggttcaaagcaattatcattagtatataaagctgtaaactcaatgttcctatactctgatgtagaaatgcaaaatacaaatcgtaaatcatggtggaaaaatgcagatcactggcaaactaatattttaaggcttataaggggtgagaataaaacctcaaaatctgtaaaactcaatggtcagaactataaagaattaaaaatctaccctggagtgagtgtcagtgcatatatgaccagcagaatctgctcatgctgtggcagaaatatttttgaattaataaaaaatgatgagcttgaggataaacataaaaaatatcaggttaacgcacagggcgagatcaatataagaggtgaagtaattaaactctatcaaaaatcagacagtcacaaaactttagtacctggacttaaaagtaaaaaaacctataacgccatcaatcaaagggcccctatggttaccccataccctgagggcattatagatattgagcagctaaaaaaaataattcgctttaatttaaggcgcgctccggcaagtcgcatgtctaaagatagtactcaaagcagatatttctgtgtgtttaagaactgtaaaaatcatcaggttgaaaaacatgcagatattaatgcagcgataaatatcggcagaagatttttaactgatattattattcataattaa

SEQ ID NO: 45 - DNA, 3789 bp, synthetic construct
  source 1..3789: mol_type = other DNA
atgctgacgaccaagttcaaactcgagcttcccgcgggatgtccgcttcgtgaggatgccgccacgtttgacgagtgccggaaactctatgacgtggtggaaggttgcggaaacggtacgctcaccggttttcttttttccgtcattctttccggctttcggatttttccggacggcaagatggccgagattttcgccaaccgttccgtctacgacgaggacgaattccggagtgctctggtcgaggcggtgggtgcgcctttgccccgatttaccgtcaaggcgctcatcaagcgacttcagatggaggtgcgggctcgcggcaacaaggacaaccgcttcgttgccgaagtcatgatgaaggaatatcggcagaccctctgcggtaagaccctccccaaaggagtcgatgaatcctacgtcgacaggctctttgaagaaatggcccgggagctgacttcgcgctaccgctcctggaacgaactcaagggcgatctgctcggcgcttgtaaagcggtggacgcggcgctccgaggcttcggagattttccttctctggcaacgatggtgacgcgggccgctgcacgtagattgccgaaggattcgacaatcgtcttcgatcctcaatcgcctttcattgacgtgcaaacgatcggggtcgatgccatgccctacgcggcagtggcgacacttctttcgtatcccgaaagtgtcggtgaaaagagacgggatttcgtccaaaatcaccttacgacgccctcggcggcgggcctgagttggctattcaatcggggactggaacttttttcggaggaaagcgtcgaagaactttgtcggctctttcatgtgccggaagatcaacggacccgcatagttcaaattcagaatgcggcccgagcgaccccgaggcaaagctttttcctgaagaagggcggcgctcccctcggataccacgattttcgaagcgcctttgccggccgcatcaacagttggacggcaaactacctcaaccgtcttgaagaactgcagggacttcttcacgatctgacggacgaacttcggttgcccgaccttgtccgaaacggcgaggactttctcgcaacaaccgactgccgccgggaagaggtggagattttgtgccgaagcttttctcgggagcgcgatcgtgcgcagacggctgtggagcatctcatcggcgccgatcccttgcaggtggtgagcgacgttgcggcgatcgaggaatacagtcgaatcgtcaaccgcctctgcgccatcaaggagcagatcgtcaattcgctccgacaggccgaggacgacaaggcttcccgttggactacgctctggtccgaggtcaaaaacgaatttcaaccgtgggagaaacttatccgtcttcctaaactcaacggaatgtcgggcggagtgcctcctgcgcaggacgaactcgagacgattcttgcccgctattctgacgtagtccgaggtgcaaccgaacattttgatgtcgtcatggagtgggcggccaaaacgggcgccgagggcgatatcctcaaaaagtttgccgagacggaacagcagcgtgccgatcaacgcgctccgggcaaatacgacggtcgggagctggcgttgcgtctcgtcctgcagcgcgtcgcccgtgtcgtgcgcgatcgttccgacgtctgcgccgagaatgttcggcagtggttcctgaaggaaaacatctttgcggagcggaaggacttcaacaagtttttcttcaatcgtcttggaagcctctatgtttcgcccttcagcaaccgccgtcacgtcggctacaaattgtccgacggactcgtggagcggtccggcgccgtttggcgcgaactgctcgccttggtgaaggagaagaggggagcctatgaagcgttctcggaagcgggcgagacgttcctgcgtcttgaaaacctcttgatggttatgagaatcggcgctctcacggagaatgttcctgcggaagtcgccgctcttcgtctcgacgaagagacagcgctcgagagcgtttccgaaggtctgaagcttcagctccaacaagccgaagtgccgccctcggtacttgccaaggccttcaacgtctatgtcagtctgctgagcggttgcttgatcgccctgcggcgcgaacgctttttccttcgcaccaagttctccttcgtcggcaacacggcgctcgtttacgttccgaaggaaaagagctggccgatgccctcgcgctacgaagcatcgccctcctggactccgattttcgaaaacgacgtgttggtgcggctctcgaccggggaagtggaggttgccgaaaccttccgtcgcgccgctgcgctctggggacgaacgacggatcccgtcctgaagaaagctttgcgggaactctttcatcagttgccgcacgactggtgttgtcaggtctctgttcggagttccggcgacatgacgccggcaaagcggaaagaggatgatcgggacgtgttgatcgtggagaagaaaggaaagtacgactctacgatcatctcgaaaaagattgcggccacggctcttgtgcgccttgtcggaccgagcacccataaagaacggctcaaccgtttgctgttggatgttggagaagtggcctgcgacatgacgttgcttgccgaccaggagatccttcagaaagtggaggacggtcgtgtaattttgtctttgggaaaactgcagttttcactttccgtgccgatttcaactccggccgaacaaagcgaggacgaagtgaagagcgagagaaaatcgacgcacttccgaagaatcgtcgccatcgaccaaggggaacgaggatttgccttcgccgtattccgattggaagacgcgggaaaggagggtgcgcagccgattgctcagggattcgtgaacattccgtccattcgccgtcttatcgcccgcgtgcactcctaccgcaagggcaagcagtccgtacaaaagttcagtcagcgctttgactccacgatgtttaccctgcgcgaaaacgtggcgggcgacgtgtgcggagccatcgccggtctcatgtgccgctaccgcgccattccggtgctcgaacggcaagtgagcaatctcgcgagcggcggcaagcagcttgaactcgtctacaagatggtcaacgcccgtttcctggacgaccgcattccgatgcacagtcttgagcgcacttcctggtggtgcggaacctccgattgggtcatccctgatttgtgggtcgaagttcccgaaagttatgccgtcaaggcaaagaaggatgagattctcaagaaggacgggaagttttaccgaaccctgcggattacgccgggggtgggcgtgaacgccaagtggacgagccgcatctgttcgcagtgcggcggcaatgcgatggaactgatcgagaaggctcgtgaagaaaaggtaaagaccgtaacgctcgacgccaacggagaagtcacgctctttggcaggacagtgcgtctttataagaggccttcggaagaacgaagcagggaagccaggcgccgcaacgagcgtgccccctggacggagccgcgtgcaaacgtcaagctttctctcgacgacttccgaagagctgtcgccgaaaacatgcgtcgccagcccaagagccttcagagccgagacacgtcgcagagtcgctacttctgtgttttcacagattgccgctgccataacaaggaacagcacgcggacatcaacgcagcagtcaacatcggacgccgttttctggagagcctgttgcgcgagtga

SEQ ID NO: 46 - DNA, 3909 bp, synthetic construct
  source 1..3909: mol_type = other DNA
atgcaaacaaagaaaacacaccttcatctaatttcagccaaggcatcaagaaaatatcgaagaacgattgcatgcttatctgatacagcaaaaaaagatctggaacgacgtaagcagtcgggagcggctgatcccgctcaggaactcagttgcttgaaaacaattaaatttaagttagaggttcctgaagggtcgaagctgccgtcttttgacagaatttcgcaaatttacaatgccctcgagacaatagagaagggctccctgtcatatcttctttttgcgttaattctctctggattcagaatttttccaaactcgtctgcagcaaagacgtttgcaagctcctcttgttataaaaacgatcaatttgcatcccagataaaagaaattttcggggaaatggtgaaaaattttattccttcggaacttgagagtattctcaaaaaaggccgcagaaaaaacaataaagattggacagaggagaatattaagagagtcctgaatagcgaatttggtaggaaaaattcggaagggtcttcggccttgtttgattcctttttaagcaaattttcacaagaattgttccggaaatttgattcatggaacgaggtcaataagaaatatctggaggctgcggaattgctggatagcatgctcgcctcatacggaccatttgattccgtttgcaagatgattggagactctgattctagaaactcgttgccagacaaaagtacgattgcttttacaaacaatgctgagatcacagtagacatcgaatcatcagtaatgccctatatggcaattgcggctttgttaagagaataccgtcaaagcaaatcaaaagcagcaccggttgcgtatgttcaaagccacttaacaacgacgaatggcaatggcttgtcatggttctttaaattcggcctcgatctcatcagaaaggctccggtatcttcaaaacaatcaacttcagatggctcgaagtctcttcaagagctattttctgtgccggacgataaattagatgggctaaagtttattaaggaggcttgtgaagcccttccagaagcttctttgctttgtggagaaaaaggagaactgctggggtatcaagacttcagaacttcttttgccggtcatatagacagttgggttgctaactacgtaaatcgcctttttgagctcatagaattagtaaatcaattaccggagtcaattaagcttccttcaatacttacgcagaagaatcataatcttgttgcatccctgggacttcaggaagcagaagttagccattcgttggagctgtttgaaggtcttgttaaaaatgttcgtcaaactttaaaaaaattggcaggcatagacatctcgagttctccgaatgaacaagatataaaagagttctatgcattttcagatgtgcttaatcggctgggttcaatccgaaatcaaattgaaaatgctgttcaaacagccaagaaggataaaattgaccttgagtcggcaatagagtggaaagagtggaaaaagttaaaaaaactcccgaaattaaatggtcttggcggaggagttccaaaacaacaggaattacttgataaagcgctggagagcgtaaaacagataagacattaccaacggatcgatttcgagagagtaatccagtgggcggtaaacgaacattgcctggaaacggttcctaaatttttggttgatgccgaaaaaaagaagattaataaagaatcttctacggattttgctgcaaaagaaaatgcggttcgcttcctacttgaggggatcggagcagctgctaggggaaaaactgactctgtaagcaaagctgcttacaattggtttgttgtaaataatttcctcgccaagaaagacctgaatcgctatttcattaattgtcaaggatgtatttataaaccaccttactcgaaacgtaggagtttggcgtttgctttaagaagcgacaataaagacacgattgaagttgtttgggaaaaatttgagactttctataaagaaatttcaaaagaaatagagaaattcaatatttttagccaggaattccaaacatttcttcacttagaaaatctgcgtatgaaacttcttttgagaagaatacaaaagccgataccagcggagatagcgtttttttctcttccacaggagtactatgactcccttcctcctaacgtagcatttcttgctttgaatcaggaaattacaccctcggagtatataacccaatttaatctatattcgagtttcctgaacggtaatcttattctgctgcgaagaagcaggtcttatctgcgagctaaatttagttgggtgggtaatagtaaacttatctatgcggcaaaagaagctagattatggaaaattcccaatgcatattggaaatcggacgaatggaagatgatcctagattctaatgtgctggtgtttgataaagccggcaatgttcttccggcaccgaccctgaaaaaggtgtgtgaacgtgaaggtgatctgaggcttttctacccacttttaagacaactcccccatgattggtgctacagaaatccctttgtgaagagcgtgggcagagaaaaaaacgtcattgaagttaacaaagaaggggaacctaaagttgcttcggctttaccgggatctttgttccgactgataggtcctgcaccttttaaatctctgctggatgattgtttctttaatcctttagataaggacctacgggaatgcatgcttattgtcgatcaggaaataagccagaaggtagaagctcagaaagtggaggcctcattggaatcatgtacttattcaatcgcagtcccgattagataccacttagaggaaccgaaggtatccaatcaatttgaaaatgttttggctattgatcagggagaagccggacttgcatatgcggtgttctctcttaagagtattggtgaagcggaaaccaagccaattgcggtcggtacaatcagaattccgtccattaggcgattgattcattctgtctcgacttacagaaaaaagaagcagcgactgcaaaactttaagcaaaattacgattcaacagccttcattatgcgtgagaacgtgacaggagatgtatgcgcaaagattgttggtttaatgaaggaattcaatgcgtttccagttctggaatatgacgttaaaaatttagagtccggaagcagacagctgtcagcagtctataaggctgtgaactcccacttcttgtactttaaggagcctggcagagatgcattaagaaagcagctttggtacggcggagattcttggactattgacggaattgagattgtgacaagggagaggaaagaggacggaaaagaaggggtggaaaagattgttccgctgaaagttttccccgggcgttccgtttctgctcgattcacgagcaagacctgttcctgctgcgggaggaatgtttttgattggctcttcactgaaaagaaagcgaagacaaataagaagtttaatgtaaacagtaaaggagaacttacgactgctgacggcgtcattcagctgtttgaagcagatcgatccaaaggtcctaagttttatgcgagaagaaaggagaggacgcccctaacaaagccaatagcaaagggctcttactctctggaagagattgaaaggcgtgtgcgcacaaatctaagaagagcccctaaatccaagcaatcgagagatacgagtcagagtcaatatttttgcgtttataaagattgtgcgcttcatttttccggcatgcaggcagacgaaaacgctgcaattaatattgggcgccggttccttactgcattgagaaaaaatcgcaggagtgattttccttcgaacgtaaagatatctgacagattgttagataattaa

SEQ ID NO: 47 - DNA, 4368 bp, synthetic construct
  source 1..4368: mol_type = other DNA
atgaaaacagagaatagggggctgatgtactcagccccctttattaaactagtggaagggaacttaatgacttcaaacgaaacaactacacaaagtaaagatttcaagaactacagacatcccagatttaatcctaaaaaagacggatctttaaaaaaagtaatacaaatcgttaaagatggtgtgcctacgtatggggacttcaaccctcatgaacaaaagaaggataaaacccctaataggagaggacatcttcttagtaggggagaaagtcgaaaattaaaacgaagtataagggctctcagcgattctcaaaaaaaaggagaaggggcctttgagcaaactcgaacaatagtatgtaaaactaagggagatgtccgaccagaagctaatttcgagttaatgcgggagacttataacactttgaacgcacttgatttcggatctctaaaattctctttcttagctcttccttttctaggaatgatagcgtcccctaaaaccgcccaatgtgtgataaatgaaaatcgtgagcattttgttcccgatggattattcgagagtatcgtaaaggacctgggagttctaggggagggtatgaccccggagaccatagcattggctgtccaggctgaacctgggaaaaaatgttgcccggataaaggaagggtagagtttaaaaaagtcctgtttcgggcattccctaaatcaatgggattttctttagagtacgtcatcaatcaaataaaaggcttaaccgctgaactaccaaaattagagtccgaagtagctgctctaaagtctgaggtcgctcagattgaaaaagacttacaagagttatcagaagagtctaaaaagtaccaaaggactgagaagagcttattgaaaaaagaatcggatttaaaaacaaaggaattaaagcttgctgaatatgagcaaaagttaacaaattataaagcagaacgggactctttctttaaagtcgatgacttcattcaagaagttttagataatgtggttgcctgtagtgaagtcaaagatagagccgattttctgaacttagacaaaaaagtctacgtttatgagtgttttgatcttgctctgaagaaacttaatccagaatactcgggtcgattaacttcactagcctcatttttcaaaaatcacaagcctaagggaagaacaatagctttcgttcctgatcttgagtacaaagggatggacttcctaaaagataatgctgatattcttccttacttaaatttctcagccctcatgaatcggttagttagcctcggtcttttaaaaagaggcgagtttagtcctaaggtgattactacctttaatgatctcgtcctatccccaaacaatgacgctcttaatagttttttaggagtggggtttgaaaaaataagaacttccagtctggaagagcttagggattactttaacgtagaatccgacaaagacgacactataaaagctcttcaagaactactcaccctggctgtagaagaccatgtctttggtaaaaaaagctattctgaatttagagtagaagtcggttctcaaattaaatccttttacagcaatcacggagctcgatgtattgcgttttataacgcttcaatcgatccttctccaatcgaagtctctggggatttatggaatctttcgaatgcctatttatttcgaaagacttatgccaaccctgaatttcttaaaaatcaaatcgagagcgtgaattcttctggagaagttcttaaaagtagattattccgtataatgggttattccgaagaacttccttctagagaagatattgagttctttaaggatttcagccgtttagtagaccgaactttttcagatatagaacgcatcaacacctctttaaaagaggaaataaagacttatgatccaaagacagataaaaaaataatcaagagtctagaggacctcataatcataaccccgaaatgggcgaaagatattaaaaagctagctggagtaggcggaggtaagaagtctgcaaaagaagaggctcaggatctgattgatcgtttcaacttaatacgctcaagactcaatactcaagtggacgctgtagtagttacagcaggcaatcttgagactatggaagtacataaacaagtttctctagacgctcttaaatcaaacctggattacgattcaaccgtggattacgacgagctctactacagagaaaccttcgactccttaataagattgattcgagatactaattgccctccattgattaaaaaatttagggacgatgtggtatcttacggcctagttcgaggtagtggcataaagccgctgaccgtgtacatcaactctgggaaaggaagagtgtttgtccaccctaaaagtaattacaaacatagtgctttgaacatcgaactgagtatccttgaaaagttcaatcctgttgcttaccttgaaggccttttagagtacttagattcaatccccaggttaaaaccaggacggctgttttgtttgaaagaaactattaggttggagatcattaaatttaagatcttcaatattccagacaccgttcccgtatcttcgattaatcaagattattttgatttgatcgagggtagaacttttttaagcgatatcacgagagataaagatgagatattgaactccgagttcaggacgatcataaactgctacataacaactttgagatctatcatacctgacgttacccaagaaggggtgtccttgaggcttacttttaggaaaaaaggaaccagtaccgtgatgggagttcctaaacacgaagaagtcactactaatgagaaaggtgaaacggagtctatcttcaagttcactttacctgcaagtttagagtattctaagggtcctttaagcactctgatctcagaattaaagaatccctcatcagtgtttaaagtcgaaagtaaagtaaccaaagagagtttccctaaaatcgaggtctcggtgggtaagggccatcaagttgtaaaactatcggagcgaagtattaaaaaactaactagcccggagttagtcgcaagcgacggtaaaaagatccccaatccatacttagaaggagtttcacaacttctaaactacataccgcaaaatctttgcattgaaaccggatggaaggtttcaaaaagcgataaggacacaagagggattactttcggcaaaaagaaactgggtttcaagtctgctccaggtgttcttaaactggtgtccggagccacgcaactgcagacggtgcaacaatctttaatagatgaggacattaacctaggcgatgtggagtatgtttttgagcagaagtataaaaagaagcttgattttagaggagatcaggtttttttaagaaatgtgaagattgaacattctaagaataaacccgaggtcgaagtttttctcaacatcccggttactgagaagaggcttattaagaaatccgcgatctctccttttaaccacaccgccgatattggatttgatctgggggagtatggtctggcttatgcggttttggatattagaacaggtgaaatcaaagctactggattcgttccaatcaagatgtttcgaaaactgataaatgtcgttaactcgtatcgaaagcacaaccagccccggagagactacagtaaattcagtgattctaaacttcagaatatgaaagaagcagctaccgctgagatttgtacgattatttggagtttgatggatcttcataacgctcttcccgtatttgagaataatgtttcaggattgtccagaggtaagaacgcagttcggaatatttacgctaacgtagtagattacttcgtcagaaattcgagcaacgcggcatcccagtctagactcaagcattctttctatggagacattaagattactcggacagatggaaagggaaagaagatattttattccccaggaagagttgtttctggagcctacacaagccaagaatgtagttgttgtggtaagaatcctgttcgaatggttagatactctgatgttgaggagtactccatcgattccgagggggcggtcattctagacggagagtttaagtattatttgaaggcaactgctaaaaataacacttcaaagcatacccgtgcagacttcaccgcagcgtttaaaccaggaggcaagatccgcaaaaaagacctcatctctaggattaaattgcaaatgcgaagggctcccgtagataaaagaacgaagaactcctcccagtcgaggtatgtctgcttgtttgatgattgttcattggtcgaaatgagtgctgacactaatgcagcaataaatattgtaaaaagatga

SEQ ID NO: 48 - DNA, 1440 bp, synthetic construct
  source 1..1440: mol_type = other DNA
atgatcaaaacatttaagaccgcggttttcaatgttaagtttagtaagagaaaaggaaatataatagatacacagatgcgtttagccgaaaacgcattctatgatgttatagaacggctggcacatcatgttgagccccttattaagctaaataaagaacaaagaaaggacatgttaacacggcttaaaaaagaggctagtcaacttataaaaccacatcctttatccaacgcctctaaatcaggtgtcgtcgcagatgcaatcgcacagattagttcaactgttgagcttcgtttaacggggcaagatgcaaaattacccacacgaaacaatagagacatcgacacttatgatattggaatggatatgttagttggttcgttagatcttgaatctcaagatttggctaagcaattaatatattcaaagccttatgatggtatgccaagacctttactatggcttaggactcggccatcggatggggctatgttgttgagagatggtttaggcagatattttgtttatattaacagtcatagttctaagtcgaagttttcgaaagcaaaagttgttattaatgatttggtgaatgttcgaacaggggaaaccgaaaacttctctagttccactggtttattgttgcccatacaactctctaagtggcatcaatctgaatttttggctaaaggtaaacctaaatcttatcgattaataaaaaaagcagacgggtacatattggcagtcacttttgaatttaaagctgaaaaaattgagcctgctacctatttgggggtagatcgcggtatagacaaaattgcggcttttgctgttacttcaaaaaaagaagttttaaagaaagatttttgtgatggcaatgaactaagagactatcaaaaagaatgtgagacaaacgctagaaaaaaacaaacaaaaggcaatgccaagtatatccgatggcgtggttacactgatttaataatgcataaaattgcaaatgagatcgtcaatacagcgctaaagtataggtcacaagtagttttggaagatttgacaaatattgcgaacggacaccatcacaggcgagctaggtttgcacgaaaaactaatttcaataaagttttaagtcgtcagcaatatcaaaaattacagcatttattaaattataagctgtcctacgttgggctaccaacaccgctatttgttagagctgcaggtacatcaattacttgcaataggtgtgggaactatgattctaaaaatcgcgacctaaatgagcgatcgttgtttttgtgtaaaagctgtaattatcaagataacgctgatgttaatgcagcagttacaatttcaatgaaaggtgaatggttgactactcagttcgataaagaacataaaaaaatgaaaaataggttctcagactggatccctttaccatcatag

SEQ ID NO: 49 - DNA, 1521 bp, synthetic construct
  source 1..1521: mol_type = other DNA
atgtcagatgtcgtcacatcgtttctgacggtgaagtacaaacttcacaatccatccaaacgccgcagagcgatgttgctggatgcgatgcgtcgcgcccatttgggatacgacaagctgctgaagcgggttcgtgaagatgttgaggcgattgtcgacatcactgaacgccaggaacgcacggacgccgagaaggagctaaccaagaaactccaggcgcttgccaagcccctcccccttgggaatggcccaaaacaggcgatcatcgcagatgcattggcccaatctaaaagttatgtcgagttgaagaaggccgacccaaatacctcatacccaaccacacctaggttgaaagtcgaccaagtagactatgacgcagcggtggatgggatagcaaactcgcagtccatccttgaagaaaatgaatatcgagacctactggcaaaactgtcgcgtcctggcctgcctcgtccactgaacatcctgaagaaccggatcggggatggcgcgctgcttttgcaagacgacaatggtcgcctgttcgttttcatcaacctgctgccgaagacggctaaacgcaaacgaaaggtcgacctgacgggtctgatcgatacccgtacaggcgagattatgcagaagtcgacatcaagcggcgatattttccccttggaatgtgggaagtggcatgacgagaagttcttaaaacaaggaacgctccagtccagtcggctgatatatgacggtaaagacttctacttcgccgcgacgttccagtttgaggcacctctacgggagccgacgaattacattggtgtagatcgtggcattgagctgctggcagcttggtcagtgatcgacgacaagggccgcaagctagatgcgggataccatggcggcgaacgcctcaggagttttcaacgcagacaagagcaagatcagaaagacacgcagcgtcggggaaagatttacaccagccgaacccgccgtgcagtcgccgatgaggaagtccacatcgtcgcaaataagatcgtggacatggcggcaaaacataacgctgtcgtcgtcctggaagacctgaagaccattacgatggggccacatcagaaacggcccaagggagccaggaagagtggcttccgacgaatgctaacccgcgcccaatatgccaagctgaagcactgcgtcgactaccggctaaagatggaagggttcgcgcctctacgtcgtaacagccccagctatatggaaattcatccggcctacaccagtctgacctgcgccaagtgtgcgcaccaagacaaggaaagcagacaatcacaggcagtcttcgtctgcaccaagtgtggacacaaagacaacgccgatgaaaacgctgcggtgaatgtcgcggcaaaaggcatccacttcgatcagatcgtgaaagggaggaagaagggacagaaactcaaagaccatgaacagttttcggcgtggtacgccgatctgaaaaacgggggtggaggccatgcagatggtccttag

SEQ ID NO: 50 - DNA, 1446 bp, synthetic construct
  source 1..1446: mol_type = other DNA
ttgaaggcaacgaccatcacaaagagcctccgcttctggatcgacgaaccgcctgagcggtgccgtctcctctatggcgtcagcgatgaactcaccgatgcatacaacgctatcctcgagtactgggagaccgatgtccgcaaggtagccgttgaggccaccgaggcggcaatcgaggcataccacgtatggaaggacgctcccaaggagacgcgcggagacaagcccaagtggtggtcagcggacacggcaaccagagcggcgttcctgcgagtcggagtcgccgcaacatcgaccatccgcactcggctagtagacaacatggtgcagggcgagtggctgcgggacgtgaagacctacatgagccgccggttcaaggaccacatcgccgccggcatccggcaccgcgcgaagggcgttgtcatctctctcgaccagagcacaatgaagccggacgtgcagctcggtgacgatgggcgctggacgatccggtgcgcgctatgggcgcagggcaccgaggagtcggcacgcaacaacgaccgatggctcctctcgccatatgaccggaagcggcagacgtggacgatcgcgaacctgctcgaagccgaccagcacggcgaggtccggggcgtcaagctagtcccgccgaagccgggcgcaccggcaggcaagcgccgatggtcggccatgatcaccgtgacgctgcccgtggagtccagcgtgctagacgaggagcgcccgaacgtggcgggcgtggacatgggcctcacgcacttcgcggtgtatagctgcccggccaggaacacattcgagttcgtctcatcgagagagttgcaggcagcgatggagaaggcgcgccgcaggcggcggggtgtccctcgcaagcggagtacggcactcggccagaagttgggcaggcgtcaggatgcgctctgccgactgaccgctaggcggctcattgattgctgccggcgtgatcgggtcgggaccctgcgcgtggaagacctgacaggcatccgcgaccagggcagcgatgatgccgaccggaacttcgcgctcggtgcgcggttcccgtactacaagctacagacgtatctagagcaagccgctgcgtcggccggggtgcggctggagaaggttcagcctgccggcacgagtcagacgtgttcgcggtgcgcggtgcgcgatcccgagtctcgggacggcaagcggttcgtttgccggcactgcggttacaaaggcgatgccgacctgaacgctgcgaacaacatagcgtcaggtcgcttccggcggtcgatccgaccgtctacacagccggcgggacgtatcccgtcggccgcgcccgactgcacgggcgaggcagtgaaggcgtcagacgttgacgctgagctgcaacctgcgcgcacgccggcagcttgtggaggtgatccatatgccggcgagtga

SEQ ID NO: 51 - DNA, 1164 bp, synthetic construct
  source 1..1164: mol_type = other DNA
atgttaaaagcgcatgtgatccgtctcaacccaaccgaagaacaggccagctacttctggcgctgcgcggggattgctcgcttcacctggaattgggcgctggctgagttgaacgccgcctatgaccggggtgaacggcctgcgattggaagtctaaagctagcgttcaaccggctgcgcaaggaggagggttttgcgccgttcgtgggagaggttcagagctatgcctaccagcaggcgttcaccgacctgcaaaaagccctgagccgttaccacgacttccgtaagcgcggtttgttgaagcctccggccggttggaaggggcgcaaggatcacaagccctttgggtggccccgcttcaaggcccgcaaccgcagcacgcccgccttctatctggcgaacaacggcgggttgcggttgcagggccaccaggtcacgatccagcgctgtccgggcggcccggtcaacatggccgagcagttgcgctttgctggcagggtcatgggcggccgcgtgcgctatcgggccgggcactggtatctgaccgtccaggtcgatgtaccggtggaaccggtgccagcgcacaccgggccggctgtcgggctggacgtaggcatcaagacgctggcagtcaccagcgacggcgagatctacgacaaccccaaggctctggggcgccaccagcgcaagctgcgcctgctgcaacggtctctggccagacagacgcgcggcgggtccaactaccgcaagacacaggccaagatcgcccggctccacgagcggatcgccaacatccgcaagcacaccttgcaccagattagccacgagatcacccgtgactatgggctgattgggctggaagacttgaacgtcgccgggatgctcaagaacggcaagttggcaaggtcgatttcggacgtcgctttcggcgagttgcgccggcagatcgggtataaaagcgagtggcgcgggtcgcgggtagtaattgtttcccgttggttcccatccagcaagacctgcaatgagtgcggccacgtcatggccgacatgccgctatcggtgcgctggtggcagtgcccgacctgcggcgcagaacatgatcgggacggcaacgcagcggtcaacatccgcaacgaggccgtgaagatggcaggcgctgcctag

SEQ ID NO: 52 - DNA, 1164 bp, synthetic construct
  source 1..1164: mol_type = other DNA
atgttaaaagcgcatgtgatccgtctcaacccaaccgaagaacaggccagctacttctggcgctgcgcggggattgctcgcttcacctggaattgggcgctggctgagttgaacgccgcctatgaccggggtgaacggcctgcgattggaagtctaaagctagcgttcaaccggctgcgcaaggaggagggttttgcgccgttcgtgggagaggttcagagctatgcctaccagcaggcgttcaccgacctgcaaaaagccctgagccgttaccacgacttccgtaagcgcggtttgttgaagcctccggccggttggaaggggcgcaaggatcacaagccctttgggtggccccgcttcaaggcccgcaaccgcagcacgcccgccttctatctggcgaacaacggcgggttgcggttgcagggccaccaggtcacgatccagcgctgtccgggcggcccggtcaacatggccgagcagttgcgctttgctggcagggtcatgggcggccgcgtgcgctatcgggccgggcactggtatctgaccgtccaggtcgatgtaccggtggaaccggtgccagcgcacaccgggccggctgtcgggctggacgtaggcatcaaggcgctggcagtcaccagcgacggcgagatctacgacaaccccaaggctctggggcgccaccagcgcaagctgcgcctgctgcaacggtctctggccagacagacgcgcggcgggtccaactaccgcaagacacaggccaagatcgcccggctccacgagcggatcgccaacatccgcaagcacaccttgcaccagattagccacgagatcacccgtgactatgggctgattgggctggaagacttgaacgtcgccgggatgctcgagaacggtaagttggcaaggtcgatttcggacgtcgctttcggcgagttgcgccggcagatcgggtataaaagcgagtggcgcgggtcgcgggtagtaattgtttcccgttggttcccatccagcaagacctgcaatgagtgcggccacgtcatggccgacatgccgctatcggtgcgctggtggcagtgcccgacctgcggcgcagaacatgatcgggacggcaacgcagcggtcaacatccgcaacgaggccgtgaagatggcaggcgctgcctag

SEQ ID NO: 53 - DNA, 1164 bp, synthetic construct
  source 1..1164: mol_type = other DNA
atgctaaaagctcacgtgattcgcctcaacccaaccaaagaacaggaaacgtatttctggcgctgcgcgggggttgcccgcttcacttggaattgggcgctggccgagttaaacgcagcttacgagaaaggcgagcggcctgccgttggaagtcttaagctggagttcaaccggctgcgtaatgaagaaggctttgcgccgtttgttggagaggttcaaagttatgcctaccagcaggcgttcggcgacctgcaaaaagccctgagcagctaccacgacttccgcaagcgcggcatgttgaaacctcctaccagttggaagggacgcaaagaccacaagccatttggctggccccgcttcaaggtccgcaatcgcagcacgcctgcattctatctggcaaacaatggcggattgcgaatgaacggccatcaggtgacaatccagcgctgccccggtggcccggtcaacatggcggaaccattgcgctttaccggcaaggtcatgggcggccgcgtgcgctatcgggccggtcactggtatctgactgtccaagtcgatgtaccagtggaaccggaccctgcacacaccgggccggctgttgggctggacgtaggcatcaaggttctggcatttaccagcgacggcgtgatctacgacaaccctaaggccctggcgcactaccagcgcaagttacgtttgttgcaacggtctctgtctagacagacgcgcggcgggtccaactatcgcaagacgcaggccaagattgcccggctccatgatcggatcgccaacattcgcaagcacgccttgcaccaagtcagtcacgagatcacgcgcgactatggactgatcgggctggaagacctgaacgtttccgggatgctcaagaatggcaaactggcccggtcgatctcagacgtggctcttggtgagttgcgccgacagatcgagtacaaggcagactggcgcgggtcgcgggtaatgatcgttagccgctggttcccgtccagtaagacatgcaacgactgcggctacgtgatggctgacatgctgctctcggtgcgctggtggcagtgcccggcctgcggtgcagaacatgatcgggacggcaacgcggcggtcaacatccgcaacgaagccgtaaagatggcaggcgctgcctag

SEQ ID NO: 54 - DNA, 1539 bp, synthetic construct
  source 1..1539: mol_type = other DNA
atgtacggcgattctcgaatcgaggaaggaaacatggacgcgctcggtaaatcgtcccgagatgggaccaatcagcatccgccttgcgacgctgcctcaaccgccgtttgcgaaacggccgtcatcgttcgtgcgactgctcgcatgacgattgacttcatggccatggaggagccggagcgcatccgcgcccggcagcttctttacgaggcacgcaggaactcggctgctgccgcgaatgcggtgcttcgagcgttctggcgtgctgacggcgacgcgctcgatggctacatggtcgagcatggccacgggccgaagaaggcggtcgactggcccatgccaaagatccagagctacgggctcgcccgtctcgtcgctccgatgcttccgagcggcattgggtcagcggtcgctcgcatggccgagaccaagtggcgccagactcggttcgaggccctggtccgaaacaccgaaaagcccgcgcactaccgcgaggccaatccgatcccgattcggcagcaggactactcgttgacgaaagaagacggtcgctggtgcctctccatgatgctccggtcgggcgagccgcgcgtgaagctcccgcttgacattcgcgactcgttccagcgttccatcttggagaacgtctccgaatggaagggcagccgcgcgctcgcgcgcaagaagggcgagagccgcgaggcgtacaagcagcgcgtcgcttcgctggaggcggctcagcgtggctgggaggctggcgagcttcggatcgagcaagactcgaagcgcaaggcgcgctggtacgtccgcatggcctacaagcgaatcgtcgacaagcaggcagacggcaagcgggcttccttacatcgaggaatcaaaaacttcctcgtctgcgtgaccgacgatggccaagagtggaagtacgagggcgcggacatcgaggcgttcctcgcgcagatgcaggcgcgtcgccgtcagtaccagcgcaacagcctcgcgtcgtcgcgaagcggccacggcaggcgcgtcatcttgcgacccatcgacaaactgcaaggcaaggctgaccgctggcgcaggaccaagaaccagaccttggcgcgcaggcttgccgagtggctgcgagaccgcggcgtctcggtgcttttcatcgaagacctctctgggattcgctccggcgagcccgagaagcttgagggcggcgagcgtatctacaagcgcgttcaggagtggcccttttacgatgcgggtcagcgcattcactcctgctgcgaagagctagggatcaacgtcaagacggtgagcccagcctacgactcgcagcgttgccctgtctgcggcgccattgacccagagcacaaggacctccgctactggaagctctcgtgcaagtcttgcggcgctcgtcgcgacctcgacgtcgccgccgcctacaacgtcctcgcgcgcgggcttgcggtccacgggggcaagggagaggattacaaggacattggccgcgccaagcgcgctgccaagggcaagaagaatgctggttga

SEQ ID NO: 55 - DNA, 1539 bp, synthetic construct
  source 1..1539: mol_type = other DNA
atgtacggcgattctcgaatcgaggaaggaaacatggacgcgctcggtaaatcgtcccgagatgggaccaatcagcatccgccttgcgacgctgcctcaaccgccgtttgcgaaacggccgtcatcgttcgtgcgactgctcgcatgacgattgacttcatggccatggaggagccggagcgcatccgcgtccggcagcttctttacgaggcacgcaggaactcggctgctgccgcgaatgcggtgcttcgagcgttctggcgtgctgacggcgacgcgctcgatggctacatggtcgagcatggccacgggccgaagaaggcggtcgactggcccatgccaaagatccagagctacgggctcgcccgtctcgtcgctccgatgcttccgagcggcattgggtcagcggtcgctcgcatggccgagaccaagtggcgccagactcggttcgaggccctggtccgaaacaccgaaaagcccgcgcactaccgcgaggccaatccgatcccgattcggcagcaggactactcgttgacgaaagaagacggtcgctggtgcctctccatgatgctccggtcgggcgagccgcgcgtgaagctcccgcttgacattcgcgactcgttccagcgttccatcttggagaacgtctccgaatggaagggcagccgcgcgctcgcgcgcaagaagggcgagagccgcgaggcgtacaagcagcgcgtcgcttcgctggaggcggctcagcgtggctgggaggctggcgagcttcggatcgagcaagactcgaagcgcaaggcgcgctggtacgtccgcatggcctacaagcgaatcgtcgacaagcaggcagacggcaagcgggcttccttacatcgaggaatcaaaaacttcctcgtctgcgtgaccgacgatggccaagagtggaagtacgagggcgcggacatcgaggcgttcctcgcgcagatgcaggcgcgtcgccgtcagtaccagcgcaacagcctcgcgtcgtcgcgaagcggccacggcaggcgcgtcatcttgcgacccatcgacaaactgcaaggcaaggctgaccgctggcgcaggaccaagaaccagaccttggcgcgcaggcttgccgagtggctgcgagaccgcggcgtctcggtgcttttcatcgaagacctctctgggattcgctccggcgagcccgagaagcttgagggcggcgagcgtatctacaagcgcgttcaggagtggcccttttacgatgcgggtcagcgcattcactcctgctgcgaagagctagggatcaacgtcaagacggtgagcccagcctacgactcgcagcgttgccctgtctgcggcgccattgacccagagcacaaggacctccgctactggaagctctcgtgcaagtcttgcggcgctcgtcgcgacctcgacgtcgccgccgcctacaacgtcctcgcgcgcgggcttgcggtccacgggggcaagggagaggattacaaggacattggccgcgccaagcgcgctgccaagggcaagaagaatgctggttga

SEQ ID NO: 56 - DNA, 2577 bp, synthetic construct
  source 1..2577: mol_type = other DNA
atgaaacaccagtacaaacccaagaaatgcaagttcatcgaacaccgtgcagtaaagttcgaccgggaaaccggcaatccgaaactggatgcaagcggggccgaaattccgttcaccgaaaaccgtaccgcggtgtgcaagattaacccgaagtccgtcgatccgagactcctggaaaccttcgatgcctccaaggaaacaatcaacgacatcctcgccaacatgtccgaacactggttcgatgtctacacggtcgaatccggtgtcaagaacgacatgaagaagttcaccatcatggacctctatgccggcgcagtccctggggacatcctgaaaggcgaattcaccctcgtccacggaaggaagcgcgtactggtgaagaagacgattaccgggtatgtcacccgtgaactcatggcgccccaggaggacgacggcttcatcctgtgcgaccgagaacagttcatcaactccctcaaccggaagacggacaagattttcggcgaggaaacctccattccggcaaagtggtggtgcgataccatctgcggcgaccttgacacgatgttgaagggttatgcccagtgcgtactcggcatgagcgataccgacgatggcaagtggaggaccgctgtccgcgaagtgtccgaaagcatctacggcaacgaattctcccggaaacatgccgagcgaaccatcatcaagctcggccctcagcacctcaggcacgtaaacggcctgatgccggacacttccgtcatccaatggcccatctcgtgcaagatctgcggtgagaatgccacaatcaccgaacccgacttcgccaaggaacctaaactcaaacgactgtacctggcctccatgaaggcattcgagcgcatcgtgaaggaatcgttcccaaagaagaacgtgttcaagccgaacatcccgatgctgcccagggattccgtcaagaaactggacggctactacaactattccgccgaactcctctatatccccggtcccaagaaggcaagtcgcttccgtgtcgaattccgggcaaagtccgaccgtaccgggaacgactactacccgaaggacctgttcaagtacacctccgagtgcataatcccgcgcttctccatgctgaaatccaccggggccatgacactcaacataccgtacaccgtcccgtgccagaagcccttcatgtcccaggatgccgaaatcaactgggacgccggcctcggcatcgacctcggatatgcaaggttcgccatggtgctctcgaaacccgcttccaagtatcccggaatggtcaactggaacgaagcccttgactggttctccaagaagtatggcctcgatgtcctcaatgcccactgctccaaggcgacccggaaggaaatcgaagacatgattgccgaggaacgggatggaaaggccaccatgggcgccatcttcctcctcggggtacgcgacgggaaccctccagacatccagcacgactggcgcccgtcccatgaccccatggccaccctgttcaccaggatggaacgcaggaccgacaaggacggctccccgttctactccgaacagcagctcgccatcatcggccacaccaagaccttccgcatccagatgcgccagatcttcgccaaccgcatcgaatactaccaccgccagtccgaatgggacctcaaccattccgaggaacaggtgttcgccagggaatccgaggtcgccaaggcccttgccgcaaggtacgacttccttaacgaatccatccgctgcattacccagaggttcatttccgacatcctgacatctgatggggcgttcaggccggcgttcatcgccatggaggacctgaacctcaacgagctggagaaggacagctccttcaagtccctatacatgacaatcacgggagactggggcatcgatccccgccaggattacaaggtctccgtccggaagggacgcaccgtcgcagaaatcacatatcccgagggcaagaagccccccaggcccgcgcagttccccaaggtgttcccggctaccgagcactggaacacccccgctaggatctccgccaagggacagaccatcgtcatcgcgtgcacccctaccagcaagggaacggtggccatggcacgcgacagcatcgagtgctacaccaagaaggcgctccatatcgccctcatcaagcacgatgtcgagcgcctgtgcacccacatgggcatcctgttccgcgaggtatccgccaagttcacatcccagacatgcgactgctgcggaaacgccaaggcggtatcccatgacccgtctgaaaatggtttcgacccctgtgcctcgatgcgggccatgaaggaagggaagaacttccgcttcaagcgtaccttcatctgcggcaacccggcgtgcccgatgtgccaggtctccgtcaatgccgacagcaacgcggcatccgtcatctgccacatggtcaggaacgggaaatccgactatttcaaggacaagcgtgccaagttcaaggcaccgaaggtccaaaaggagacaaagaaatcatctaagtccaagaaggacaagtag

SEQ ID NO: 57 - DNA, 1287 bp, synthetic construct
  source 1..1287: mol_type = other DNA
atggccaaaggtaccaagaatactgatattctctatcggtccgaaaagtttgaattgttttggaatcgtaggccggtttgtgcgccaaccgcagaagagctggccttgctcacaatcaccagtgaaaacttgcggacagtatggaacgaagcctggcgtgcccgcatggatgcttacgaaaatttcttcaagcctatttacgaaaaaatcgggacagcaaaaaagcttcaagacgaggctttggtaaaagggctttatggagaacttcgcgatgccttcaagcagcatggtgtgagtttgtacgaccagattaacgcactaactccgcgccgtaaggctgaccctgcctttgccagtatcccgcgtaactggcaggaagaaacgctggattgcctggatgcttcgttcaaatcgttttttgcccttcgcaaaaacggcgatgcggatgccaagcaaccttttgcccgtgaaacgcccggatttttctgtaaaatcccggggcgttacggattttcgtttgatggtgaaaacatcaccatcagttttgcgggcttgggtcagcgaattgtttgtcaggtacccgaccatcagcgtgaaaggtttggaaattccctgcgtttgaaaaagtttgagatttaccgcgatgagcgcgacctttcaaaaccagggagcttctggatttccgttgcctacgaaatcccaaagccgcccgaaaagcctgttactcctgataacacggtgtacttggctcttggtgcatcttaccttggcatggtttgcccgaagggtgagttttacttccgcttgcctcgtccggattttcactggaagccattagttgaccaagtgcaggaacggctgaaaaacgtcactaagggttcccggaaatggaaaaagaggatatcagcccggtggcggatgtttgacattctgggcaaacagcaaaaacaaggccagtacgaactgatccaggaagaactgctttcacagggtgtccattttgtcatcaccgacttggtggtgcgcagtaaaactggagctttggccgatgcttccaaaccggagcgaggcggagcgccaaccggtgcaaattggtcagcccaaaatacgggctggattgccaatttggtagccaagctggccgaaaaagccaaggagcacggcggtattgttgttaagcgcgagccatcgcagctttctccccaagaacggaaaatgcaccccggcgaacgcaaaatcatcattgcccgaaggatgcgagaagcgtttcttgccgatcaacagtaa

SEQ ID NO: 58 - DNA, 1632 bp, synthetic construct
  source 1..1632: mol_type = other DNA
atggaaacacaatattataaggttgcagcatacccgatgaaaatccggctgtaccccacaaaggaacaggcgaagaccatagacagctggcttctgggcttgcagaaggcatataatatgacgctgtacgcgctgaaagagggcgtgccggaacttcgacaaaagtcaaaggacggttcaacagaatttccaaattggaagtacatcggcagtaaagcatggctggattctttgcgggaaagaagcagttatgttgcaagtgtccccggtggctgtctttcttctagcgttggcggcgcgttgggggcggatattaaaaaagcttgggaaagtcagggaaagctcccggttgacgcatggttcaaagcgacggatgcaaaaggacattccattattcgctggtattcggatagcagaccccgaaaaagttgttttttccaaattgaggcaaacaggttcacaagaacaaatcagagtgtgtatataacactgcggaaggatttcacaatcaaagcaagaggctggaacgacaagatacgttttgcagcggattcaacggaaagcttttttgaaagataccgggacggcaatgatgttttcagtttcagaattagtagagataactgtggcgattattatgcagtgattacactgaaggatgtttaccgaccgtttaacgtggaaccggaacggaggggcattggaattgatgctggcgtaaatgccatggcaacagattctgatggcgtttcttatgagaaccctcgtatcaaaaagaaaaatgagacgttaaaggcggaatttgggaggcaaatggcccgacggtatggcatcaaaaatgaacaattccgaaaagagcgaaaagagactcgaaagtataatacattgcacaacgaggagattgcgaatcaaacggttgagccaagatgtatttctccatcaaaacgctacttgaaagcacaggtaaagctatctgagctggaacgtaaggtaagacgacagcgggaccttattcagcatacctacactgcgcggattatagcgaaagcaaatctagtagcgattgaaaatctgaatgtgaagggtatgatgggcaattccaatttggcagatagcctgtcggatgcggcaatgtcagagttcttgaggaagctgaaatataaggcgcagtggtcaggcggagaatatcatgcaatcgggacatttaccgcttccacagagcgctgtgcccaatgcggctatgtcttgcagggaacggaaaagctcacgcttgccgaccgtatttttacctgcccgatttgcggaaacacggatgaccgggatgccaatgcagcaaaatccattttggaaatcgcggaagaagaaatcaaagagggcatcccatctgcggataccgtcaaaaagccaaaggaaaagaaaaagaaaacctacccggataagccaatcgggaaaaattatcctgatgtatttacgcatttctcagaagaacttgttcaacagcataaaaatccgtttgtaattgtaaatgataggcaagaagtattggatgatgcccagggatatgggtacagtgacagacagagtgcccagaaattctggacacataaaatgaaaacacaacaaaaacagtaa

SEQ ID NO: 59 - DNA, 1632 bp, synthetic construct
  source 1..1632: mol_type = other DNA
atggaaacacaatattataaggttgcagcatacccgatgaaaatccggctgtaccccacaaaggaacaggcgaagaccatagacagttggcttctgggcttgcagaaggcatataatatgacgctgtacgcactgaaagagggcgtgccggaacttcgacaaaagtcaaaggacggttcaacagaatttccaaattggaagtacatcggcagtaaagcgtggctggattctttgcgggaaagaagcagttatgttgcaagtgtccccggtggctgtctttcttctagcgttggcggcgcgttgggggcggatattaaaaaagcttgggaaagtcagggaaagctcccggttgacgcatggttcaaagcgacggatgcaaaaggacattccattattcgctggtattcggatagcagaccccgaaaaagttgttttttccaaattgaggcaaacaggttcacaagaacaaatcagagtgtgtatataacactgcggaaggatttcacaatcaaagcaagaggctggaacgacaagatacgttttgcagcggattcaacggaaagcttttttgaaagataccgggacggcaatgatgttttcagtttcagaattagtagagataactgtggcgattattatgcagtgattacactgaaggatgtttaccgaccgtttaacgtggaaccggaacggaggggcattggaattgatgctggcgtaaatgccatggcaacagattccgatggcgtttcttatgagaaccctcgtatcaaaaagaaaaatgagacgttaaaggcggaatttgggaggcaaatggcccgacggtatggcatcaaaaatgaacaattccgaaaagaacgaaaagaggctcgaaagtataatacattgcataatgaggagattgcgaatcaaacggctgagccaagatatatttctccatcaaaacgctacttgaaagcacaggtaaagctatctgagctggaacgtaaggtaagacgacagcgggaccttgttcagcatacctacactgcgcggattatagcgaaagcaaatctagtggcgattgaaaatctgaatgtgaagggtatgatgggcaattccaatttggcagatagcctgtcagatgcggcaatgtcagagttcttgaggaagctgaaatataaggcgcagtggtcaggcggagaatatcatgcaatcgggacatttaccgcttccacagagcgctgtgcccaatgcggctatgtcttgcagggaacggaaaagctcacgcttgccgaccgtatttttacctgcccgatttgcgggaacacggatgaccgggatgccaatgcagcaaaaaccattttggaaatcgcggaagaagaaatcaaagagggcatcccatctgcggataccgtcaaaaagccaaaggaaaagaaaaagaaaacctacccggataagccaatcgggaaaaattatcctgatgtatttacgcatttctcagaagaacttgttcaacagcataaaaatccgtttgtaattgtaaatgataggcaagaagtattggatgatgcccagggatatgggtacagtgacagacagagtgcccagaaattctggacacataaaatgaaaacacaacaaaaacagtaa

SEQ ID NO: 60 - DNA, 1620 bp, synthetic construct
  source 1..1620: mol_type = other DNA
gtggaagttcagtacaataaaattgatgtttacccgatgaaaataaggctttaccctacaaaagaacaggcaaaaacaatcgatgggtggctaatgggattgcaaaaggcgtataacatgacgctgtatgccctgaaagagggggtgccggagcttcgccaaaaatctaagagtggagatgcagaattccccaactggaagtatattggaaaacgggagtggctggatgagctgagaaagaatagcccgtgtgtggctaatgtgcctggggggtgcttgtcatctacggttggcggcgcactgggggcagatatgcggaaggcatgggagagccagggaaaactgcctgtggatgcttggtttcgtgcaactgacgcaaatgggcgacatattgtacgctggtattcggacgctaaaccaagaaaaagttgctttttccagattgaggcggggaagataagcagagaaaagcaaagtgtgtatattacactacgtaaggattttcgagtaaaagcgcgggggtggaatgacaaaatccgtttttcagaggagtcagatgaaggcttttttgagaaataccgggacagtaagaaagtgcttagtttgagagtgagtagggataattgcggagattattttgccactatcacattaaaggatgtatatcgaccgacaaaggttgaagctgaaagaaaaggagtcggaattgatgttggggttcgcgcaatggcaacggattcggatgggagggtttacgagaacccccgaataaagcaaaaatttgaggataaaaaaacggagttggggcgacagctgtcccgacgctatggggcgaagaacaagcaattccggcaggattgcaaagaggcgcgaagctttaataggttgcacgagtcagaaatcaatgaaaagcttgtagttccaaagacggttttgccatcgaggcgttataatcaagcacaattgaagctgtctaaattggaacgtaaaatgcggcgaaaacgagatatggcgcagcatgtctattcagcagaagttgtcaggaaggcaagtttggttgccattgaaaacttgaatgtgaagggaatgatggccgattccaatctcgcagacagattgtccgatgcggctatgtctgagcttttgcgcaaactcaaatacaaagcggagtggtcgggagccagttaccatgcgattggtacatttactgcatcaacacagcggtgtgcgaaatgtggatatatcctgcggggagaaaataaattggcgcgtgatgattccgtttttgtatgcccggtctgcagaaatgtggatggccgggacgctaacgcagcaaaatcgattttgcaagttgcacagaaggaaatttccgagggggttccttcggcggatttgataaagaaattggaggtgaagaaaacaaaaacctacccggataagcctattggtaaggcttttccaaatgtttttacccattttagcgaggagtataagaagcaaaagagaaatccatttgtaattgttgatgagaataaaaatgtcctgaatgatgcccaaggatatgggtatcgtaacagacagagtgcgcagaaattctgggcacataaaatgaaccaataa

SEQ ID NO: 61 - DNA, 1674 bp, synthetic construct
  source 1..1674: mol_type = other DNA
atggaaacacaatattataaggttgcagcatacccgatgaaaatccggctgtaccccacaaaggaacaggcgaagaccatagacagctggcttctgggcttgcagaaggcatataatatgacgctgtacgcactgaaagagggtgtgccggaagaaccggaagaaccggaagaaccgggcgagaagaagccagaactgcgccgaaaatcagaggatggttcaacggagttccccaactggaagtatatcggaagtggggactggattgagcggctggaaaggaaaaaccagtctctcaaaggaatcccgcatgatgcgtactataatgccgtcggcggaatgctttcggttgacatgaagaaggcgtgggaaagtcaggggaaattacctgttgataaatggtttcaggccagggatgagaagggccacttgattgtacgatggtataacaaggggaagacgagaagttccgcctaccttcaggttgaagccagaaaaattgttcaacagggcaagagcgtttttataaccctgcaaaaagggttcagcgtcaaggcgaggggctggaatgaaaaaattcggttttcagaggatttgacgcaaagcttttttgacaaatatcagggggacaaaagaaaagtaggtgtgagaatcagcagggataattgcggtgactattacgctgtaatttcactgaaagatgtttatcgccctgttaaagtagaagcagagagaagaagcgtcggtgttgatgcgggaacaagagtaatggcgacagattccgacggcatgacttatgagaatccgcggataaagaaaagaaatgaagctgaaaaggcggaactggaccgacagctggcacggcgatttggagccagaaacgaacagttccgtgcggaatgtaaagcagctcgcaaatacaacaagtcgcatcaagaagaaattgcagaaaaagtggttgagctcaagagagttttaccatcgaagcgctacttaaaggcacagttaaaattgtccaaattggagcggaaggcggcacgacagcgggatatggttcagcatatccatacagcgcaaattgtggcaaaagccaatctcgttgccatcgagcacttaaatgtcgaaggtatgaaaaaggattcaaattctgcaagcagtgtagaagatgcagcaatgtcgggacttctgcaaaaaataaagtacaaagcgcagtggtctggtggcaactatcattcaatagggacatttgacccctctacacagcggtgtgcgcaatgtgggtatgttttgaaggatgaagaaaaactaaagcgaggggacgatacgtttgtttgtccgcgctgcggcaatgtggatggccgagacgaaaacgcagcaaaatctatactgatagttgcgaaagaacgtattgaaagaggacttccatctgcggacaccatcaagaagccaaaggaaaagaaaaagaaaacctacccggataagccaatcgggaaaaactatcctggtgtattcacgcatttttcagaggaatttagacaacaatataaaaatccgtttgtaattgtaaatgataagcaggaagtattggatgatgcccaggggtatgggtacagtgatagacagagtgcccagaaattctggacacataaaatgaaaacacaacaaaaacagtaa

SEQ ID NO: 62 - DNA, 1872 bp, synthetic construct
  source 1..1872: mol_type = other DNA
atgaaaacaatcaaaatcaaaatcaaactcaccactgatcaggtgcaactgtgcgatcgctatttggaagaattgacatggctgtggaatttaacactttctaatcaactacataatcactgtgtgacgtggtacgattgggcggctaaactcagtgctaatttggataaagctactgagaaattagataaacttaaaccagaacaacagcagttaataaaggattattacaggactaaggataagcctaagctatctaaaaaagaacaggaattagtggctaaatttgacatatttgctagatggaattcatttagtctagacgggattattcccgttcctttacggctggggaatagcggttatgaaggtttatcctgtcagatagctacgggtggtaactactggaagagagatgaaaatatcaatatcccaattaataccaagaaaggtattgttcacgttaaaggatacaaattagttaaaggtgataaaccttggcaacgaatagaaatagtaccccataagtaccgtacattccccggtggaaagtttgaaggtagggaactgactactttagagaaacttgataacgtgaatggactgaacactttaagggcattccaaaatttaccagacctacaagtttcctcccactatataggcggactactggcattttttaaagaatcatggtcggcatttttagaccctaaaaggatgaacagtagaaagcctaaattcaaaaaagacagtgacaagattacaactttatctaacaatcagtgtgcgcctaacagaattgacgttaataaaaacatagttactgtcactgggtttagtcctatcgccattattgataaaaattgggtaaaaagattgaacttgtctgaagttctaccacgcacatatatgttaacccaaaatccatcagggtactacattaatattgtcattgctcatccattacatgaagagaaaaccgcgctggtaaagaaattacccaaagttaaaaaagagtttggagaagatagtcaagagtatgaagatatcaagtcaaaaatcaaatttcttgaacaacaaatcaaggaagcatcaatagttaaaggcaaagatttaagtgtgggcattgaccccggagtacaggctgttgtgtccacagatcatggtgctttattccttcctaaccttaccagagagcgggtttcaattcatattgaggaattacaatcacgcttagacaatattgaattaattaacgataaaaagtggaaaagcttgggcaacaagacccctagaatcaagacaaagaatgaaactaagttacaggaaaaaataagccgtctacacgaacgtggggctaattcatccaatgcttttaatcataagctatccacaagattatctcgtacttatgaacatattgcctgggaagacacgcagattaataatctactaaaacaagtagagccaaaagcattaccagaaggtataggatatgctcacaatggtgcatctgctaaacgcggtttgaattggattatgagacaaagatgtttgagtgatttgaaagctaaaaccaaacagaaaacagaaaatagaggtggcaacttccatgaaccacctgctaactatagttctcaaacttgtcattgctgtaaacaaaagggagaacggcgatcgcaacatgaatttatctgcaaaaactctgactgtaaattatttgatattccccagcaagctgatactaacgctgctagaaatcataagcagaatggtggttttgaactgggagaggtcaagtatcataatgttaagctagtttatcaaaagcctaaaagatttaagaaaaaacgcttgacaaaccaatag

SEQ ID NO: 63 - DNA, 2502 bp, synthetic construct
  source 1..2502: mol_type = other DNA
atgaccaaggctgagcgccttcgcgacgtagcgaagaaccggctcgtccggctggagcgacgtgcgcaaaagattggcgaacatctaaagcggaaaccgggcgacctgcaagcgatgcaccatctcctgcatcaaatcgaagtcgagtaccacgacgtctcgcgcaatctaaaggatcctgactgggttcctaagccaaagcgcaagcgagagaagcgcaatatcgagagtaccgaccatatcccgccccccacgaagggcgaccctggcgtgccaaagcactattcaataccgaagcccgtgcctctgccggttgaccgaatcccggaagaccagctcaagatgggatggaagagcagcggacgctcttggtgttctcccccgttcgtagaggttgccctaccgcccgggaaagagcacgtcgttattgaccatctgtcaaagttcaagattgacgacgatagggaagtcgtgcgtgcctgggccgagaaagagtttgggtcgatcgcggttgctaaagaagcccttaaggttggcgctacgctttcggttgatgctggcgtttggcgcggcttgattgagcaggcgggtaagtcagagcgcttcagtgacctcaccggcgaagaactgcttgcagacgcctctgcgcggaggatcgcgatggcctggcaccaattcgaatgggtgaagcaaacggcaaaatcaatcaccgactctgcgccgaaaggagtctcgaaggatgctttggcgtcgaaaacaagggcacacctcaaatcgtttcacacagcggtgaactcgttcaagcaggttacgaacaaggagacgggcgctgtcgaacttgtgaacaagcacaccaacccgcagttttcgtacctgagcatggaaaagccggtagtcgatgcggacaccgtaagcgagcaagtcgtcgactggctctcactcccggtcgacgaacgcttcaccaaagacgaaaatgatccgaagaagcgggggcgaatcaccgtcttgcaaaaggaactgggcgctgcaaagcggtcaaagcactggcgaggccgcgaacagacgcagccttgggcaggcaagcctcattggaagggcacgctcatcaggaagcgcgacgcattgctgatttatgactgttgcaagaatggcctcgccctcgtgctttgcacaggcgggggagtgcgtgtcgacgtggaatcactgctgaatatggatggcactgacctccggtcggatcgtcaactccttaccccaaacggtaccggaaagtcggcgttcgtgttgcccttggtccctaagcatgattttcaccgttggtatgcgaagcacgtcgaaaatgacaatgcagacgcaccgctgaccaagcgatgtatccacaacaccactcagttcgtggtcattcccgagcacaaagggcatccgccgcaactatttattcgaccggtcctcaaattctacgatcccggaaaggagattcccgacacccacgcttggggagcgaaacctcagtgtcggtatctcattggcgtggaccgcggcatcaactcaccctattttgctgctgtctatgacacggaacggaacgccatcgtagccatacgacagggtcgcggacgaaaggacgaatggaagaatcttcgaaacgagctcgctctggcacagcgcgaacacaacgaacttcgcaataagcgaggcaaagcgaagcagctggcaaaggcgatggccaacattcgtgcattgcgaaagaaggagcgagggctgaataaagtagagaccgtggagtcgattgccgaactcgcgaactgggccgaaaaagaattgggcgcatgcaactactgttttgtgattgaggagcttcagcagatgaacctcaggcggaataatcgggttaagaatattgcggcaatcaaggacgcccttgtcaatcaaatgcgaaagaagggctacaaatacaaggagaaaagtgggaaggtagacggcgttcgcgaagaatcaccgtggcatacgagcgctgtttcgccctttggttggtgggcgaagtcagaagatgtcgataaagacaagcggttcatcgggcgtcgcgtcggcggacattactgccgcgacgctgaggatgggcgctacatccgaggaatgtataagaaaccaggcggcaagtacggacgaaaagtcttcactctctctgaggacgatttgagaaccgggattcgacgccgctcattcggctcggagcttttctgggacccgcatcggaccgagtttcgtggcaagccgtttcccaacggtgttgtcctgaacgctgactttgtcggggcgttcaacattgccgtgcgccccgtggtgaaggacggcaagggtaagggctttactgcgaagaacatggcggaggtacacaccgagttcaatccaacggtggctatcgagtgtgagatacccctatacgagttcaccgaagtggacggcgatccccttggcgcccttcgcgaagtggttgtataa

SEQ ID NO: 64 - DNA, 2574 bp, synthetic construct
  source 1..2574: mol_type = other DNA
ttgagcccgctcgaacggtccttgcggaaggtcggtgagaatcgccttgagcggctgcgggtgcgagaggagaagattaggaagcacatagaacagcacccccgcggtaagaacgatcatcaggctctccacttcttattgcaccaaatcgaggtcgagcgtaacgacctgtaccgaaacctcaaagaccccgagtacgtgcccaaaccagcgaaacagcggcgcgaaagacggcagatcaacgtcgccaaacccccgactcgaccaaagaaggaaaaggggcctcaaccagagtcgacgaagtacgtgatccgtccaccagtccctgggaaaaaccttcctgcctttgctagcaagtacgaggcgcgagacacgcgggacgattcctaccaggacggtcgctcatggacctccgcaccatatgttgaagtcgaacttcccatccttggtgcagacaaagtcatccagaaactgatgaagttcgtgcagaaggacgagcggtcgatcgtgcgcgactgggcgacaaagacgtatagctcgatcgaagccgcaagagaagcactccttgtcggggcacaagtctcggaagacgtttcggtctggcgcggactcctcgcagaaacgaagaacgcacagaacttcgccgccctctccgacgatcagatcgaagcagcgatgtcgaaggaggcgaagggcgcggacttgcgtccgaggcgcgccgcactgctggtcgcacagcgccactgggtggatcagaccgtcaaagcaatcaaggagtccgcaccgtccggcgtcgacaaggacactctcgatcgccgtctgcgcgcaggtctgagggggtttcatactgcggccaactcaggcaagcacacgaacccgcagttcccatacctcaccgcagagaagccggtagtcccgatggagtctgttgttcagagcgtattggcctttctcgacgatccagacgatcaaaggtacacgaaggacaaagaagacgacaagaagcgccaccgcgtcactgtcttgcagaaggagctcggaaaggcgaggccacgaaaacggttagaactccaaacgccgaaatgggccggcaggcccacggtaaaaggaaccatcagcaaacggcgcgacgcagcgctcgtctgggacacaagcaaagaagcgaacgggctttgtctcgcgctcccaatcgggggcatgccgaagatagacgtcgagcagttcatctaccaggatgggacgtcgctcctgtccgattgccagatcgcatcgaaaacgaccaagaagggcgcggcttgcgcagtcttgccgctcaagcccaagcatgacttcctgcgctggttcaccaagcacgtcgagaaccacaatcccgacgctccactggaacgcaggtgcctccacaacacgacccagttcgtcatagtcgacccagaagggccgcgcccacgtctcttcgtccggcccgtcttcaagttctacgaccccggcaagacggtgccgaacacgcatgaaacttggaaaaagcccgactgccgctacctggttggaatcgaccgaggcatcaattacgttctgcgagccgtcgtcgtcgatactgaagagaagaaggttatcgccgatatcggcttgccgggcaggaagcacgaatggaggatgatccgtgacgagatcgcctaccaccaacagatgcgtgatcttgcccgcaacactggcaaacacgcgagcgtcgtggccaagcacgtccgcgccctcgcgctcgcgcgcaagaaggaccgcgcgctcggcaagttcgcaacagtcgaagccgtcgcagaacttgtcaagaagtgtgaacaggactatggtagcggcaactactgtttcgtgctcgaagacctcgacatgggggcgatgaatctcaagcgaaacaacagagtcaaacacatggcggtcatggaggaggccctcgtcaatcaaatgcgcaagcagggctatgcctatgacgggcgtcgcggtcgggtggacggcgtgaggcacgagggcgcttggtacacgagccaggtctcgccctttggctggtgggccaagcgcgacgaagtcgaggaggcgtggaagagggacaagactcgccccatcgggcgcaaggtcggcaactggtacgagatgcccgagccaggccaagacggagaccggcccgacacgtatcggaagggctactggtcgaaaccgaagaacgcggagggcaagccgtatgggcgcaaccgcttcagcgtcgagcctggcgacgagaagccggacgctgagcggcgcttctgctggggcagcgagctgttctgggatccgaacgtgaagtccttcaagggcaaggagtttcccgagggcgtcgtgctggacgccgacttcgtaggagccctcaacatcgctctccgcccgttggtcaacgacggccagggtaaaggcttcaaggccgaggacatggcgagggagcacacgatactaaacccgcagttcaagatcgcctgccagataccagtttacgagttcgtcgaagaggacggcgacaagtgggcagctctgcgccggatcatgctatag

SEQ ID NO: 65 - RNA, 36 nt, synthetic construct
  source 1..36: mol_type = other RNA
ggtgcaatcgcccggatttcgcgacctgcttacagg

SEQ ID NO: 66 - RNA, 21 nt, synthetic construct
  source 1..21: mol_type = other RNA
atttcgcgacctgcttacagg

SEQ ID NO: 67 - RNA, 32 nt, synthetic construct
  source 1..32: mol_type = other RNA
gtcgcatcttgcgtaagcgcgtggattgaaac

SEQ ID NO: 68 - RNA, 25 nt, synthetic construct
  source 1..25: mol_type = other RNA
cttgcgtaagcgcgtggattgaaac

SEQ ID NO: 69 - RNA, 36 nt, synthetic construct
  source 1..36: mol_type = other RNA
gccgcagagctcggaaagtcagagaaggtatggcgg

SEQ ID NO: 70 - RNA, 11 nt, synthetic construct
  source 1..11: mol_type = other RNA
aggtatggcgg

SEQ ID NO: 71 - RNA, 36 nt, synthetic construct
  source 1..36: mol_type = other RNA
gcgccactcgctgatgcttgtgttactgaatggcgg

SEQ ID NO: 72 - RNA, 14 nt, synthetic construct
  source 1..14: mol_type = other RNA
ttactgaatggcgg

SEQ ID NO: 73 - RNA, 36 nt, synthetic construct
  source 1..36: mol_type = other RNA
gctgtagaagggcgtccattcacggctgacggaaac

SEQ ID NO: 74 - RNA, 36 nt, synthetic construct; identical to SEQ ID NO: 73
  source 1..36: mol_type = other RNA

SEQ ID NO: 75 - RNA, 37 nt, synthetic construct
  source 1..37: mol_type = other RNA
gttgcaacggcttccggagtgctggtgggaatgaaac

SEQ ID NO: 76 - RNA, 37 nt, synthetic construct; identical to SEQ ID NO: 75
  source 1..37: mol_type = other RNA

SEQ ID NO: 77 - RNA, 36 nt, synthetic construct
  source 1..36: mol_type = other RNA
gctgtagaggggcgtccattcacggctgacggaaac

SEQ ID NO: 78 - RNA, 36 nt, synthetic construct; identical to SEQ ID NO: 77
  source 1..36: mol_type = other RNA

SEQ ID NO: 79 - RNA, 34 nt, synthetic construct
  source 1..34: mol_type = other RNA
tgtataagggcgtccattcacggctgacggaaac

SEQ ID NO: 80 - RNA, 34 nt, synthetic construct; identical to SEQ ID NO: 79
  source 1..34: mol_type = other RNA

SEQ ID NO: 81 - RNA, 36 nt, synthetic construct; identical to SEQ ID NO: 77
  source 1..36: mol_type = other RNA

SEQ ID NO: 82 - RNA, 36 nt, synthetic construct; identical to SEQ ID NO: 77
  source 1..36: mol_type = other RNA

SEQ ID NO: 83 - RNA, 36 nt, synthetic construct; identical to SEQ ID NO: 77
  source 1..36: mol_type = other RNA

SEQ ID NO: 84 - RNA, 11 nt, synthetic construct
  source 1..11: mol_type = other RNA
ctgacggaaac

SEQ ID NO: 85 - RNA, 34 nt, synthetic construct
  source 1..34: mol_type = other RNA
tgtagaagggcgtccattcacggctgacggaaac

SEQ ID NO: 86 - RNA, 34 nt, synthetic construct; identical to SEQ ID NO: 85
  source 1..34: mol_type = other RNA

SEQ ID NO: 87 - RNA, 25 nt, synthetic construct
  source 1..25: mol_type = other RNA
cctcatcaatcctatcaataatgag

SEQ ID NO: 88 - RNA, 25 nt, synthetic construct
  source 1..25: mol_type = other RNA
cctcatcaatcctatcaataatgag

SEQ ID NO: 89 - RNA, 25 nt, synthetic construct
  source 1..25: mol_type = other RNA
ccttcaaaaccctgtcacatctgga

SEQ ID NO: 90 - RNA, 25 nt, synthetic construct
  source 1..25: mol_type = other RNA
ccttcaaaaccctgtcacatctgga

SEQ ID NO: 91 - RNA, 25 nt, synthetic construct
  source 1..25: mol_type = other RNA
ccttataaacccttccaataatggg

SEQ ID NO: 92 - RNA, 25 nt, synthetic construct
  source 1..25: mol_type = other RNA
ccttataaacccttccaataatggg

SEQ ID NO: 93 - RNA, 25 nt, synthetic construct
  source 1..25: mol_type = other RNA
cttagaatcttatgacgaactgagg

SEQ ID NO: 94 - RNA, 19 nt, synthetic construct
  source 1..19: mol_type = other RNA
atcttatgacgaactgagg

SEQ ID NO: 95 - RNA, 28 nt, synthetic construct
  source 1..28: mol_type = other RNA
gttcacggttacgtaggtgatatggaag

SEQ ID NO: 96 - RNA, 28 nt, synthetic construct
  source 1..28: mol_type = other RNA
gttcacggttacgtaggtgatatggaag

SEQ ID NO: 97 - RNA, 37 nt, synthetic construct
  source 1..37: mol_type = other RNA
ggtttggtcaccggcgatttgtggggtgactgtgaca

SEQ ID NO: 98 - RNA, 18 nt, synthetic construct
  source 1..18: mol_type = other RNA
tgtggggtgactgtgaca

SEQ ID NO: 99 - RNA, 26 nt, synthetic construct
  source 1..26: mol_type = other RNA
ctctccacgcgcgcgcgggatgcggg

SEQ ID NO: 100 - RNA, 26 nt, synthetic construct
  source 1..26: mol_type = other RNA
ctctccacgcgcgcgcgggatgcggg

SEQ ID NO: 101 - RNA, 29 nt, synthetic construct
  source 1..29: mol_type = other RNA
gttcaccccacaggcgcgtggagtgatgg

SEQ ID NO: 102 - RNA, 29 nt, synthetic construct
  source 1..29: mol_type = other RNA
gttcaccccacaggcgcgtggagtgatgg

SEQ ID NO: 103 - RNA, 29 nt, synthetic construct
  source 1..29: mol_type = other RNA
gttcaccccacaggcgcgtggagtgatgg

SEQ ID NO: 104 - RNA, 29 nt, synthetic construct
  source 1..29: mol_type = other RNA
gttcaccccacaggcgcgtggagtgatgg

SEQ ID NO: 105 - RNA, 29 nt, synthetic construct
  source 1..29: mol_type = other RNA
gttcaccccacgggtgcgtggagtgatgg

SEQ ID NO: 106 - RNA, 29 nt, synthetic construct
  source 1..29: mol_type = other RNA
gttcaccccacgggtgcgtggagtgatgg

SEQ ID NO: 107 - RNA, 36 nt, synthetic construct
  source 1..36: mol_type = other RNA
gtcgcaacggatgaatggatgctgattgatggaagg

SEQ ID NO: 108 - RNA, 26 nt, synthetic construct
  source 1..26: mol_type = other RNA
atgaatggatgctgattgatggaagg

SEQ ID NO: 109 - RNA, 36 nt, synthetic construct; identical to SEQ ID NO: 107
  source 1..36: mol_type = other RNA

SEQ ID NO: 110 - RNA, 26 nt, synthetic construct
  source 1..26: mol_type = other RNA
atgaatggatgctgattgatggaagg

SEQ ID NO: 111 - RNA, 39 nt, synthetic construct
  source 1..39: mol_type = other RNA
tatggtagaggtgccaccggtttacatggcgccgatacc

SEQ ID NO: 112 - RNA, 39 nt, synthetic construct; identical to SEQ ID NO: 111
  source 1..39: mol_type = other RNA

SEQ ID NO: 113 - RNA, 40 nt, synthetic construct
  source 1..40: mol_type = other RNA
tggaacggcctcctcaaagccaaccgagggggtaggctac

SEQ ID NO: 114 - RNA, 40 nt, synthetic construct; identical to SEQ ID NO: 113
  source 1..40: mol_type = other RNA

SEQ ID NO: 115 - RNA, 31 nt, synthetic construct
  source 1..31: mol_type = other RNA
taacaatctgcggatggatgtgaactgcaag

SEQ ID NO: 116 - RNA, 26 nt, synthetic construct
  source 1..26: mol_type = other RNA
atctgcggatggatgtgaactgcaag

SEQ ID NO: 117 - RNA, 32 nt, synthetic construct
  source 1..32: mol_type = other RNA
gtaacaatctgcggatggatgtgaactgcaag

SEQ ID NO: 118 - RNA, 24 nt, synthetic construct
  source 1..24: mol_type = other RNA
ctgcggatggatgtgaactgcaag

SEQ ID NO: 119 - RNA, 32 nt, synthetic construct
  source 1..32: mol_type = other RNA
gtcacaacctatgtgtggttatgaactgcaag

SEQ ID NO: 120 - RNA, 23 nt, synthetic construct
  source 1..23: mol_type = other RNA
tatgtgtggttatgaactgcaag

SEQ ID NO: 121 - RNA, 33 nt, synthetic construct
  source 1..33: mol_type = other RNA
ggtaacaatctgcggatggatgtgaactgcaag

SEQ ID NO: 122 - RNA, 24 nt, synthetic construct
  source 1..24: mol_type = other RNA
ctgcggatggatgtgaactgcaag

SEQ ID NO: 123 - RNA, 37 nt, synthetic construct
  source 1..37: mol_type = other RNA
cttgcaactgggcttggggactgaggatagttgaaac

SEQ ID NO: 124 - RNA, 37 nt, synthetic construct; identical to SEQ ID NO: 123
  source 1..37: mol_type = other RNA

SEQ ID NO: 125 - RNA, 38 nt, synthetic construct
  source 1..38: mol_type = other RNA
gcctcagggggatataagacactctaaaggaatgaaag

SEQ ID NO: 126 - RNA, 13 nt, synthetic construct
  source 1..13: mol_type = other RNA
aaaggaatgaaag

SEQ ID NO: 127 - RNA, 37 nt, synthetic construct
  source 1..37: mol_type = other RNA
gtcgcaggggatcaagaacgctcttagggaatgaaag

SEQ ID NO: 128 - RNA, 20 nt, synthetic construct
  source 1..20: mol_type = other RNA
acgctcttagggaatgaaag

SEQ ID NO: 129 - RNA, 154 nt, synthetic construct
  source 1..154: mol_type = other RNA
tttcgttcgaggccgggagcaacggaccgctagcccaagggcaaccgcggccgcccggtcgaaggccgacctgtacggcctgaaggttgagaaggcacgaataagcggaaaaactcgtttcccttcgtgttcgctcaccgagcagacgtcgctt

SEQ ID NO: 130 - RNA, 143 nt, synthetic construct
  source 1..143: mol_type = other RNA
gagaggggtattctgttgatgggcgaatcggtaaaagcaataaaattaaagatactggatatgtttttagaccccgaatgcacaaagcaggatgataactggcgcaaagatttgtctactatgtccagattctgcgctgaagc

SEQ ID NO: 131 - RNA, 143 nt, synthetic construct; identical to SEQ ID NO: 130
  source 1..143: mol_type = other RNA

SEQ ID NO: 132 - RNA, 168 nt, synthetic construct
  source 1..168: mol_type = other RNA
gagaggggtattctgttgatgggcgaatcggtaaaagcaataaaattaaagatactggatatgtttttagaccccgaatgcacaaagcaggatgataactggcgcaaagatttgtctactatgtccagattctgcgctgaagcggggaatatgtgtctgcgcgacctg

SEQ ID NO: 133 - RNA, 168 nt, synthetic construct; identical to SEQ ID NO: 132
  source 1..168: mol_type = other RNA

SEQ ID NO: 134 - RNA, 149 nt, synthetic construct
  source 1..149: mol_type = other RNA
tttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagt

SEQ ID NO: 135 - RNA, 149 nt, synthetic construct; identical to SEQ ID NO: 134
  source 1..149: mol_type = other RNA

SEQ ID NO: 136 - RNA, 121 nt, synthetic construct
  source 1..121: mol_type = other RNA
tttactctgtttcgcgcgccagggcagttaggtgccctaaaagagcgaagtggccgaaaggaaaggctaacgcttctctaacgctacggcgaccttggcgaaatgccatcaataccacgcg

SEQ ID NO: 137 - RNA, 121 nt, synthetic construct; identical to SEQ ID NO: 136
  source 1..121: mol_type = other RNA

SEQ ID NO: 138 - RNA, 60 nt, synthetic construct
  source 1..60: mol_type = other RNA
gtcgctatcagcggcaatgcaatttcctccgcctttggaaatttacgttctatgactttg

SEQ ID NO: 139 - RNA, 60 nt, synthetic construct; identical to SEQ ID NO: 138
  source 1..60: mol_type = other RNA

SEQ ID NO: 140 - RNA, 90 nt, synthetic construct
  source 1..90: mol_type = other RNA
gtcgctatcagcggcaatgcaatttcctccgcctttggaaatttacgttctatgactttgcgagagctgcggtgccccgttgcggccggg

SEQ ID NO: 141 - RNA, 90 nt, synthetic construct; identical to SEQ ID NO: 140
  source 1..90: mol_type = other RNA

SEQ ID NO: 142 - RNA, 123 nt, synthetic construct
  source 1..123: mol_type = other RNA
atacgtcgcgacattccagcatgtaacccgtcgggaggattgccccatgaaccgtatttaccaaggtcggattaccggcattcttgattccaaggaggacgagcggggccaccctcccccacc

SEQ ID NO: 143 - RNA, 123 nt, synthetic construct; identical to SEQ ID NO: 142
  source 1..123: mol_type = other RNA

SEQ ID NO: 144 - RNA, 153 nt, synthetic construct
  source 1..153: mol_type = other RNA
atacgtcgcgacattccagcatgtaacccgtcgggaggattgccccatgaaccgtatttaccaaggtcggattaccggcattcttgattccaaggaggacgagcggggccaccctcccccacccgatccgaaacacaatccattctggcggca

SEQ ID NO: 145 - RNA, 153 nt, synthetic construct; identical to SEQ ID NO: 144
  source 1..153: mol_type = other RNA

SEQ ID NO: 146 - RNA, 117 nt, synthetic construct
  source 1..117: mol_type = other RNA
cttgacatggcttaagaatgcgccatgtatgaaggagaagtaacctgcttccttcctcgctgtcaccgccttcgtcagcgatttcatcggcggaacataataattttacttttttct

SEQ ID NO: 147 - RNA, 117 nt, synthetic construct; identical to SEQ ID NO: 146
  source 1..117: mol_type = other RNA

SEQ ID NO: 148 - RNA, 91 nt, synthetic construct
  source 1..91: mol_type = other RNA
ggtatatccgttttgttctgctctctatcgcattcggggggcaatgcgtcaaagattagtgacgtgtttgcgaaatagacgtctattgata

SEQ ID NO: 149 - RNA, 91 nt, synthetic construct; identical to SEQ ID NO: 148
  source 1..91: mol_type = other RNA

SEQ ID NO: 150 - RNA, 63 nt, synthetic construct
  source 1..63: mol_type = other RNA
aaagtgtacgtctttttgtacaacctcttcggccttctggctacgggtgccctggatcacgcc

SEQ ID NO: 151 - RNA, 63 nt, synthetic construct; identical to SEQ ID NO: 150
  source 1..63: mol_type = other RNA

SEQ ID NO: 152 - RNA, 93 nt, synthetic construct
  source 1..93: mol_type = other RNA
aaagtgtacgtctttttgtacaacctcttcggccttctggctacgggtgccctggatcacgcctttgggacctgggaaaccacaacctttccc

SEQ ID NO: 153 - RNA, 93 nt, synthetic construct; identical to SEQ ID NO: 152
  source 1..93: mol_type = other RNA

SEQ ID NO: 154 - RNA, 44 nt, synthetic construct
  source 1..44: mol_type = other RNA
aaatctcggtcgatggaaatgttgccctctttggtgatgtgata

SEQ ID NO: 155 - RNA, 44 nt, synthetic construct; identical to SEQ ID NO: 154
  source 1..44: mol_type = other RNA

SEQ ID NO: 156 - RNA, 53 nt, synthetic construct
  source 1..53: mol_type = other RNA
cgattccacctaactgtctgacaggaaactgcttcgccgcattgatgcttcga

SEQ ID NO: 157 - RNA, 53 nt, synthetic construct; identical to SEQ ID NO: 156
  source 1..53: mol_type = other RNA

SEQ ID NO: 158 - RNA, 57 nt, synthetic construct
  source 1..57: mol_type = other RNA
aaattatcggccgggacggctcctttgccccagggcaggtgagcccgtagtttcgcg

SEQ ID NO: 159 - RNA, 57 nt, synthetic construct; identical to SEQ ID NO: 158
  source 1..57: mol_type = other RNA

SEQ ID NO: 160 - RNA, 87 nt, synthetic construct
  source 1..87: mol_type = other RNA
aaattatcggccgggacggctcctttgccccagggcaggtgagcccgtagtttcgcggccgggtactggacggataggcgaatgacg

SEQ ID NO: 161 - RNA, 87 nt, synthetic construct; identical to SEQ ID NO: 160
  source 1..87: mol_type = other RNA

SEQ ID NO: 162 - RNA, 102 nt, synthetic construct
  source 1..102: mol_type = other RNA
gttcgatctctttcaacaatggttgaaggaaattatcggccgggacggctcctttgccccagggcaggtgagcccgtagtttcgcggccgggtactggacgg

SEQ ID NO: 163 - RNA, 102 nt, synthetic construct; identical to SEQ ID NO: 162
  source 1..102: mol_type = other RNA

SEQ ID NO: 164 - RNA, 59 nt, synthetic construct
  source 1..59: mol_type = other RNA
gttcccagccttccaggaatggttggtcgggatcagacgccgggttggtgagccagctt

SEQ ID NO: 165 - RNA, 59 nt, synthetic construct; identical to SEQ ID NO: 164
  source 1..59: mol_type = other RNA

SEQ ID NO: 166 - RNA, 89 nt, synthetic construct
  source 1..89: mol_type = other RNA
gttcccagccttccaggaatggttggtcgggatcagacgccgggttggtgagccagctttaatcggccggtgagccaggtagtttcatc

SEQ ID NO: 167 - RNA, 89 nt, synthetic construct; identical to SEQ ID NO: 166
  source 1..89: mol_type = other RNA

SEQ ID NO: 168 - RNA, 100 nt, synthetic construct
  source 1..100: mol_type = other RNA
agcttaggctgaagggaggcgctgcattctgagtcgggtccggagctgttgcaacggcttccggagtgctggtgggaatgaaacatgatctcttttctcc

SEQ ID NO: 169 - RNA, 100 nt, synthetic construct; identical to SEQ ID NO: 168
  source 1..100: mol_type = other RNA

SEQ ID NO: 170 - RNA, 135 nt, synthetic construct
  source 1..135: mol_type = other RNA
gttcgatctctttcaacagtggttgaaggagcttatcggccgggacggttcctctgccttcgggcaagggaaccagtagtttcgcggccgggtactgggcggataggcgaatgacggcagcctcacggctggcgc

SEQ ID NO: 171 - RNA, 135 nt, synthetic construct; identical to SEQ ID NO: 170
  source 1..135: mol_type = other RNA

SEQ ID NO: 172 - RNA, 165 nt, synthetic construct
  source 1..165: mol_type = other RNA
gttcgatctctttcaacagtggttgaaggagcttatcggccgggacggttcctctgccttcgggcaagggaaccagtagtttcgcggccgggtactgggcggataggcgaatgacggcagcctcacggctggcgcgtaaccgaccatcccctggtccgaacagcc

SEQ ID NO: 173 - RNA, 165 nt, synthetic construct; identical to SEQ ID NO: 172
  source 1..165: mol_type = other RNA

SEQ ID NO: 174 - RNA, 100 nt, synthetic construct
  source 1..100: mol_type = other RNA
gttcgatctctttcaacagtggttgaaggagcttatcggccgggacggttcctctgccttcgggcaagggaaccagtagtttcgcggccgggtactgggc

SEQ ID NO: 175 - RNA, 100 nt, synthetic construct; identical to SEQ ID NO: 174
  source 1..100: mol_type = other RNA

SEQ ID NO: 176 - RNA, 100 nt, synthetic construct
  source 1..100: mol_type = other RNA
gttcgatctctttcagcaatggttgaaggagcttatcggccgggacggttctgttgccctcgggcaggggaaccagtagtttcgcggccgggtactgggc

SEQ ID NO: 177 - RNA, 100 nt, synthetic construct; identical to SEQ ID NO: 176
  source 1..100: mol_type = other RNA

SEQ ID NO: 178 - RNA, 138 nt, synthetic construct
  source 1..138: mol_type = other RNA
gaagttcgatctctttcaacagtggttgaaggagcttatcggccgggacggttccgctgccccagggtagtggaaccagtagtttcgcggccgggtactgggcggataggcgaatgacggcatcctcacggttgccgc

SEQ ID NO: 179 - RNA, 138 nt, synthetic construct; identical to SEQ ID NO: 178
  source 1..138: mol_type = other RNA

SEQ ID NO: 180 - RNA, 168 nt, synthetic construct
  source 1..168: mol_type = other RNA
gaagttcgatctctttcaacagtggttgaaggagcttatcggccgggacggttccgctgccccagggtagtggaaccagtagtttcgcggccgggtactgggcggataggcgaatgacggcatcctcacggttgccgcgtaaccgaccatcccctggtccgaacagcc

SEQ ID NO: 181 - RNA, 168 nt, synthetic construct; identical to SEQ ID NO: 180
  source 1..168: mol_type = other RNA

SEQ ID NO: 182 - RNA, 103 nt, synthetic construct
  source 1..103: mol_type = other RNA
gaagttcgatctctttcaacagtggttgaaggagcttatcggccgggacggttccgctgccccagggtagtggaaccagtagtttcgcggccgggtactgggc

SEQ ID NO: 183 - RNA, 103 nt, synthetic construct; identical to SEQ ID NO: 182
  source 1..103: mol_type = other RNA

SEQ ID NO: 184 - RNA, 135 nt, synthetic construct
  source 1..135: mol_type = other RNA
gttcgatctctttcaactgtggttgaaggagcttatcggccgggacggttccattgcctcagggcaggggaaccagtagtttcgcggccgggtactgggcggataggcgaatgatggcatcctcacggttgccgc

SEQ ID NO: 185 - RNA, 135 nt, synthetic construct; identical to SEQ ID NO: 184
  source 1..135: mol_type = other RNA

SEQ ID NO: 186 - RNA, 165 nt, synthetic construct
  source 1..165: mol_type = other RNA
gttcgatctctttcaactgtggttgaaggagcttatcggccgggacggttccattgcctcagggcaggggaaccagtagtttcgcggccgggtactgggcggataggcgaatgatggcatcctcacggttgccgcgtaaccgaccatcccctggtccgaacagcc

SEQ ID NO: 187 - RNA, 165 nt, synthetic construct; identical to SEQ ID NO: 186
  source 1..165: mol_type = other RNA

SEQ ID NO: 188 - RNA, 152 nt, synthetic construct
  source 1..152: mol_type = other RNA
atatctcaagccatgttcaaaacggcgcgtttcaaggttcacaatccgtcacggcacaagagcactatgctctggtatgccatgacccgctatcacgagactttgaaggacgtactcgaaaagacactggcgattccagatctgctagaaca

SEQ ID NO: 189 - RNA, 152 nt, synthetic construct; identical to SEQ ID NO: 188
  source 1..152: mol_type = other RNA

SEQ ID NO: 190 - RNA, 162 nt, synthetic construct
  source 1..162: mol_type = other RNA
atatctcaagccatgttcaaaacggcgcgtttcaaggttcacaatccgtcacggcacaagagcactatgctctggtatgccatgacccgctatcacgagactttgaaggacgtactcgaaaagacactggcgattccagatctgctagaacaaatctcagaa

SEQ ID NO: 191 - RNA, 162 nt, synthetic construct; identical to SEQ ID NO: 190
  source 1..162: mol_type = other RNA

SEQ ID NO: 192 - RNA, 99 nt, synthetic construct
  source 1..99: mol_type = other RNA
gttcgatctctttcaactgtggttgaaggagcttatcggccgggacggttccattgcctcagggcaggggaaccagtagtttcgcggccgggtactggg

SEQ ID NO: 193 - RNA, 100 nt, synthetic construct
  source 1..100: mol_type = other RNA
gttcgatctctttcaactgtggttgaaggagcttatcggccgggacggttccattgcctcagggcaggggaaccagtagtttcgcggccgggtactgggc

SEQ ID NO: 194 - RNA, 73 nt, synthetic construct
  source 1..73: mol_type = other RNA
caaattcggtgcaagggcgaagcccaaccggtagactcatatcttccggtaacgcacctcggctgtttagacc

SEQ ID NO: 195 - RNA, 73 nt, synthetic construct; identical to SEQ ID NO: 194
  source 1..73: mol_type = other RNA

SEQ ID NO: 196 - RNA, 103 nt, synthetic construct
  source 1..103: mol_type = other RNA
caaattcggtgcaagggcgaagcccaaccggtagactcatatcttccggtaacgcacctcggctgtttagaccagaggatggtcggttacgcggcaaccgtga

SEQ ID NO: 197 - RNA, 103 nt, synthetic construct; identical to SEQ ID NO: 196
  source 1..103: mol_type = other RNA

SEQ ID NO: 198 - RNA, 100 nt, synthetic construct
  source 1..100: mol_type = other RNA
gttcgatatctttcaacagtggttgaaggagcttatcggccgggacggttcctctgccccagggcagttgaacccgtagtttcgcggccgggtactgggc

SEQ ID NO: 199 - RNA, 100 nt, synthetic construct; identical to SEQ ID NO: 198
  source 1..100: mol_type = other RNA

SEQ ID NO: 200 - RNA, 71 nt, synthetic construct
  source 1..71: mol_type = other RNA
attgatgaggctgtctgacttgtgcaggctcattattgataggattgatgaggatgacttaaaacgcgaaa

SEQ ID NO: 201 - RNA, 71 nt, synthetic construct; identical to SEQ ID NO: 200
  source 1..71: mol_type = other RNA

SEQ ID NO: 202 - RNA, 101 nt, synthetic construct
  source 1..101: mol_type = other RNA
attgatgaggctgtctgacttgtgcaggctcattattgataggattgatgaggatgacttaaaacgcgaaactcattattgataggattgatgaggatttt

SEQ ID NO: 203 - RNA, 101 nt, synthetic construct; identical to SEQ ID NO: 202
  source 1..101: mol_type = other RNA

SEQ ID NO: 204 - RNA, 89 nt, synthetic construct
  source 1..89: mol_type = other RNA
acaatccctcttgagcattggatgcagattcttctgtctccgtacctcgctcataagggcgctgtcgggatagcagtgcctatcaaaac

SEQ ID NO: 205 - RNA, 89 nt, synthetic construct; identical to SEQ ID NO: 204
  source 1..89: mol_type = other RNA

SEQ ID NO: 206 - RNA, 119 nt, synthetic construct
  source 1..119: mol_type = other RNA
acaatccctcttgagcattggatgcagattcttctgtctccgtacctcgctcataagggcgctgtcgggatagcagtgcctatcaaaactatcccactataatgcttgtttctttacct

SEQ ID NO: 207 - RNA, 119 nt, synthetic construct; identical to SEQ ID NO: 206
  source 1..119: mol_type = other RNA

SEQ ID NO: 208 - RNA, 38 nt, synthetic construct
  source 1..38: mol_type = other RNA
tattgataggattgatgaggtcgagcatcgaaaagatt

SEQ ID NO: 209 - RNA, 38 nt, synthetic construct; identical to SEQ ID NO: 208
  source 1..38: mol_type = other RNA

SEQ ID NO: 210 - RNA, 68 nt, synthetic construct
  source 1..68: mol_type = other RNA
tattgataggattgatgaggtcgagcatcgaaaagattctcattattgataggattgatgaggcttaa

SEQ ID NO: 211 - RNA, 68 nt, synthetic construct; identical to SEQ ID NO: 210
  source 1..68: mol_type = other RNA

SEQ ID NO: 212 - RNA, 99 nt, synthetic construct
  source 1..99: mol_type = other RNA
ctcaaactccttccggctttcggaccgtctgcttgcagacctccttaccttggccggagggacatcgccgggggtcgatgcctgtcacttacccccatt

SEQ ID NO: 213 - RNA, 99 nt, synthetic construct; identical to SEQ ID NO: 212
  source 1..99: mol_type = other RNA

SEQ ID NO: 214 - RNA, 129 nt, synthetic construct
  source 1..129: mol_type = other RNA
ctcaaactccttccggctttcggaccgtctgcttgcagacctccttaccttggccggagggacatcgccgggggtcgatgcctgtcacttacccccatttcacccacttcttggcttggtttctccggg

SEQ ID NO: 215 - RNA, 129 nt, synthetic construct; identical to SEQ ID NO: 214
  source 1..129: mol_type = other RNA

SEQ ID NO: 216 - RNA, 106 nt, synthetic construct
  source 1..106: mol_type = other RNA
gcactgacacgcccagaaatgacagggttctaatcggctgatcccgccttgaattgaccagaggccgtaaggattcaaaaggacttcaatgcggcctcatgtctct

SEQ ID NO: 217 - RNA, 106 nt, synthetic construct; identical to SEQ ID NO: 216
  source 1..106: mol_type = other RNA

SEQ ID NO: 218 - RNA, 136 nt, synthetic construct
  source 1..136: mol_type = other RNA
gcactgacacgcccagaaatgacagggttctaatcggctgatcccgccttgaattgaccagaggccgtaaggattcaaaaggacttcaatgcggcctcatgtctctctccggttcgaccggctacgggaaggttct

SEQ ID NO: 219 - RNA, 136 nt, synthetic construct; identical to SEQ ID NO: 218
  source 1..136: mol_type = other RNA

SEQ ID NO: 220 - RNA, 108 nt, synthetic construct
  source 1..108: mol_type = other RNA
tcagaaactcccgacttcctcaggatgaagttgattctttatccataccttggtgccgggacgccgattgaggaatgggcggcgccttccaaattctcattcctcact

SEQ ID NO: 221 - RNA, 108 nt, synthetic construct; identical to SEQ ID NO: 220
  source 1..108: mol_type = other RNA

SEQ ID NO: 222 - RNA, 138 nt, synthetic construct
  source 1..138: mol_type = other RNA
tcagaaactcccgacttcctcaggatgaagttgattctttatccataccttggtgccgggacgccgattgaggaatgggcggcgccttccaaattctcattcctcactttcctcctgcgaaacttcatcgaagcaaat

SEQ ID NO: 223 - RNA, 138 nt, synthetic construct; identical to SEQ ID NO: 222
  source 1..138: mol_type = other RNA

SEQ ID NO: 224 - RNA, 108 nt, synthetic construct
  source 1..108: mol_type = other RNA
cagaaactcccgacttcctcaggatgaagttgattctttatccataccttggtgccgggacgccgattgaggaatgggcggcgccttccaaattctcattcctcactt

SEQ ID NO: 225 - RNA, 108 nt, synthetic construct; identical to SEQ ID NO: 224
  source 1..108: mol_type = other RNA

SEQ ID NO: 226 - RNA, 138 nt, synthetic construct
  source 1..138: mol_type = other RNA
cagaaactcccgacttcctcaggatgaagttgattctttatccataccttggtgccgggacgccgattgaggaatgggcggcgccttccaaattctcattcctcactttcctcctgcgaaacttcatcgaagcaaatt

SEQ ID NO: 227 - RNA, 138 nt, synthetic construct; identical to SEQ ID NO: 226
  source 1..138: mol_type = other RNA

SEQ ID NO: 228 - RNA, 134 nt, synthetic construct
  source 1..134: mol_type = other RNA
actagttaaagaggagaatagattatggaactaacagaagaacagcaccaagatttcatttggacttggccctcggtccactccagaggtaaagccagtctctggttaccttattttcaaggtttagagaagtt

SEQ ID NO: 229 - RNA, 134 nt, synthetic construct; identical to SEQ ID NO: 228
  source 1..134: mol_type = other RNA

SEQ ID NO: 230 - RNA, 164 nt, synthetic construct
  source 1..164: mol_type = other RNA
actagttaaagaggagaatagattatggaactaacagaagaacagcaccaagatttcatttggacttggccctcggtccactccagaggtaaagccagtctctggttaccttattttcaaggtttagagaagttgggtcgcggtaagccttaccagtataagtt

SEQ ID NO: 231 - RNA, 164 nt, synthetic construct; identical to SEQ ID NO: 230
  source 1..164: mol_type = other RNA

SEQ ID NO: 232 - RNA, 64 nt, synthetic construct
  source 1..64: mol_type = other RNA
agttcgaaggtacttgttgattgatcgggtcaaaccaataatcagcctaggaacaattttgaat

SEQ ID NO: 233 - RNA, 64 nt, synthetic construct; identical to SEQ ID NO: 232
  source 1..64: mol_type = other RNA

SEQ ID NO: 234 - RNA, 54 nt, synthetic construct
  source 1..54: mol_type = other RNA
atggggtttggtatatcttccgctccattatcttggtgtaaagtgtatgagcct

SEQ ID NO: 235 - RNA, 54 nt, synthetic construct; identical to SEQ ID NO: 234
  source 1..54: mol_type = other RNA

SEQ ID NO: 236 - RNA, 98 nt, synthetic construct
  source 1..98: mol_type = other RNA
atggggtttggtatatcttccgctccattatcttggtgtaaagtgtatgagcctatcttggccttgctctactctaggttagaaagtgctctcaacct

SEQ ID NO: 237 - RNA, 98 nt, synthetic construct; identical to SEQ ID NO: 236
  source 1..98: mol_type = other RNA

SEQ ID NO: 238 - RNA, 110 nt, synthetic construct
  source 1..110: mol_type = other RNA
agaagggacagaaactcaaagaccatgaacagttttcggcgtggtacgccgatctgaaaaacgggggtggaggccatgcagatggtccttagttacagcctccgtggact

SEQ ID NO: 239 - RNA, 110 nt, synthetic construct; identical to SEQ ID NO: 238
  source 1..110: mol_type = other RNA

SEQ ID NO: 240 - RNA, 140 nt, synthetic construct
  source 1..140: mol_type = other RNA
agaagggacagaaactcaaagaccatgaacagttttcggcgtggtacgccgatctgaaaaacgggggtggaggccatgcagatggtccttagttacagcctccgtggactgcgttgcgggtgtatgaccggaagtttctt

SEQ ID NO: 241 - RNA, 140 nt, synthetic construct; identical to SEQ ID NO: 240
  source 1..140: mol_type = other RNA

SEQ ID NO: 242 - RNA, 68 nt, synthetic construct
  source 1..68: mol_type = other RNA
agaagggacagaaactcaaagaccatgaacagttttcggcgtggtacgccgatctgaaaaacgggggt

SEQ ID NO: 243 - RNA, 68 nt, synthetic construct; identical to SEQ ID NO: 242
  source 1..68: mol_type = other RNA

SEQ ID NO: 244 - RNA, 73 nt, synthetic construct
  source 1..73: mol_type = other RNA
ctacacagccggcgggacgtatcccgtcggccgcgcccgactgcacgggcgaggcagtgaaggcgtcagacgt

SEQ ID NO: 245 - RNA, 73 nt, synthetic construct; identical to SEQ ID NO: 244
  source 1..73: mol_type = other RNA

SEQ ID NO: 246 - RNA, 45 nt, synthetic construct
  source 1..45: mol_type = other RNA
ctacacagccggcgggacgtatcccgtcggccgcgcccgactgca

SEQ ID NO: 247 - RNA, 45 nt, synthetic construct; identical to SEQ ID NO: 246
  source 1..45: mol_type = other RNA

SEQ ID NO: 248 - RNA, 100 nt, synthetic construct
  source 1..100: mol_type = other RNA
atggcaggcgctgcctagtgcgcccgtcgcggtagtaatcacgtcgagacgcaaaacgcctggggacggtgtaggtagcaaaccggatgaaccagggatt

SEQ ID NO: 249 - RNA, 97 nt, synthetic construct
  source 1..97: mol_type = other RNA
atggcaggcgctgcctagtgcgcccgtcgcggtagtaatcacgtcgagacgcaaaacgcctggggacggtgtaggtagcaaaccggatgaaccagga

SEQ ID NO: 250 - RNA, 97 nt, synthetic construct; identical to SEQ ID NO: 249
  source 1..97: mol_type = other RNA

SEQ ID NO: 251 - RNA, 33 nt, synthetic construct
  source 1..33: mol_type = other RNA
gcagtgcccgacctgcggcgcagaacatgatcg

SEQ ID NO: 252 - RNA, 33 nt, synthetic construct; identical to SEQ ID NO: 251
  source 1..33: mol_type = other RNA

SEQ ID NO: 253 - RNA, 63 nt, synthetic construct
  source 1..63: mol_type = other RNA
gcagtgcccgacctgcggcgcagaacatgatcgggacggcaacgcagcggtcaacatccgcaa

SEQ ID NO: 254 - RNA, 63 nt, synthetic construct; identical to SEQ ID NO: 253
  source 1..63: mol_type = other RNA

SEQ ID NO: 255 - RNA, 117 nt, synthetic construct
  source 1..117: mol_type = other RNA
atggcgaaaccacgaaccaaacaagagtacggcccgtttagcgtgcgtctgcctgttgagatgcgccagcagatcgaaacgttggcagaacaagagatgcggtcgcttcactcgatg

SEQ ID NO: 256 - RNA, 117 nt, synthetic construct; identical to SEQ ID NO: 255
  source 1..117: mol_type = other RNA

SEQ ID NO: 257 - RNA, 110 nt, synthetic construct
  source 1..110: mol_type = other RNA
gcagcgtcgcggggttttaatgggccgacttcccggcctgaagccgaggtagcagccatgtacggcgattctcgaatcgaggaaggaaacatggacgcgctcggtaaatc

SEQ ID NO: 258 - RNA, 110 nt, synthetic construct; identical to SEQ ID NO: 257
  source 1..110: mol_type = other RNA

SEQ ID NO: 259 - RNA, 77 nt, synthetic construct
  source 1..77: mol_type = other RNA
tcgaggaaggaaacatggacgcgctcggtaaatcgtcccgagatgggaccaatcagcatccgccttgcgacgctgcc

SEQ ID NO: 260 - RNA, 77 nt, synthetic construct; identical to SEQ ID NO: 259
  source 1..77: mol_type = other RNA

SEQ ID NO: 261 - RNA, 115 nt, synthetic construct
  source 1..115: mol_type = other RNA
aatgggccgacttcccggcctgaagccgaggtagcagccatgtacggcgattctcgaatcgaggaaggaaacatggacgcgctcggtaaatcgtcccgagatgggaccaatcagc

SEQ ID NO: 262 - RNA, 115 nt, synthetic construct; identical to SEQ ID NO: 261
  source 1..115: mol_type = other RNA

SEQ ID NO: 263 - RNA, 130 nt, synthetic construct
  source 1..130: mol_type = other RNA
gcagcgtcgcggggttttaatgggccgacttcccggcctgaagccgaggtagcagccatgtacggcgattctcgaatcgaggaaggaaacatggacgcgctcggtaaatcgtcccgagatgggaccaatc

SEQ ID NO: 264 - RNA, 130 nt, synthetic construct; identical to SEQ ID NO: 263
  source 1..130: mol_type = other RNA

SEQ ID NO: 265 - RNA, 83 nt, synthetic construct
  source 1..83: mol_type = other RNA
gaaaccataggtagaggcgccaccaccttacatggtgccgataccgctccgttggtgcagtgtggactgtaatggtagaggct

SEQ ID NO: 266 - RNA, 83 nt, synthetic construct; identical to SEQ ID NO: 265
  source 1..83: mol_type = other RNA

SEQ ID NO: 267 - RNA, 83 nt, synthetic construct
  source 1..83: mol_type = other RNA
ttaataatctgattacggctgattgccgccggtagaggtgccaccgccttacatgacactgataccttatatccagccgtatt

SEQ ID NO: 268 - RNA, 83 nt, synthetic construct; identical to SEQ ID NO: 267
  source 1..83: mol_type = other RNA

SEQ ID NO: 269 - RNA, 135 nt, synthetic construct
  source 1..135: mol_type = other RNA
aagaatactgatattctctatcggtccgaaaagtttgaattgttttggaatcgtaggccggtttgtgcgccaaccgcagaagagctggccttgctcacaatcaccagtgaaaacttgcggacagtatggaacgaa

SEQ ID NO: 270 - RNA, 135 nt, synthetic construct; identical to SEQ ID NO: 269
  source 1..135: mol_type = other RNA

SEQ ID NO: 271 - RNA, 133 nt, synthetic construct
  source 1..133: mol_type = other RNA
tatgctcattgaaaacaaggcatgagaaaaatacccccggttttgggccggaatggaatgtttttctcactgccataccgtttttatgaggcaaccctttagggcactgcattgggcagtgtttatgcgctgt

SEQ ID NO: 272 - RNA, 133 nt, synthetic construct; identical to SEQ ID NO: 271
  source 1..133: mol_type = other RNA

SEQ ID NO: 273 - RNA, 117 nt, synthetic construct
  source 1..117: mol_type = other RNA
cttatcgtgaggtgcagccacgatgtgcgaatctaaagtggacagaaatgggaacagacgttcgcacgaatgttggcggatttcttcggaaatcgagccaatcatcatatccatatt

SEQ ID NO: 274 - RNA, 126 nt, synthetic construct
  source 1..126: mol_type = other RNA
cttatcgtgaggtgcagccacgatgtgcgaatctaaagtgaacagaaatgggaacagacgttcgcacgaatgttggcggatttcttcggaaatcgagccaataatcatatccatattaccgggttg

SEQ ID NO: 275 - RNA, 108 nt, synthetic construct
  source 1..108: mol_type = other RNA
gtgaggtgcagccacgaggtgcgaataggaagtacgcagcaatgtgctgaatcgttcgcacgaaaattggcatttttgaaaaccaaagccaataatcatatccataaa

SEQ ID NO: 276 - RNA, 162 nt, synthetic construct
  source 1..162: mol_type = other RNA
cttatcgtgaggtgcagccacgatgtgcgaatctaaagtgaacagaaatgggaacagacgttcgcacgaatgttggcggatttcttcggaaatcgagccaataatcatatccatattaccgggttgctctatagatagtgttgggtacacttgctgaacaca

SEQ ID NO: 277 - RNA, 162 nt, synthetic construct; identical to SEQ ID NO: 276
  source 1..162: mol_type = other RNA

SEQ ID NO: 278 - RNA, 171 nt, synthetic construct
  source 1..171: mol_type = other RNA
cttatcgtgaggtgcagccacgatgtgcgaatctaaagtgaacagaaatgggaacagacgttcgcacgaatgttggcggatttcttcggaaatcgagccaataatcatatccatattaccgggttgctctatagatagtgttgggtacacttgctgaacacaagatattgc

SEQ ID NO: 279 - RNA, 171 nt, synthetic construct; identical to SEQ ID NO: 278
  source 1..171: mol_type = other RNA

SEQ ID NO: 280 - RNA, 130 nt, synthetic construct
  source 1..130: mol_type = other RNA
cttatcgtgaggtgcagccacgatgtgcgaatctaaagtgaacagaaatgggaacagacgttcgcacgaatgttggcggatttcttcggaaatcgagccaataatcatatccatattaccgggttgctct

SEQ ID NO: 281 - RNA, 130 nt, synthetic construct; identical to SEQ ID NO: 280
  source 1..130: mol_type = other RNA

SEQ ID NO: 282 - RNA, 253 nt, synthetic construct
  source 1..253: mol_type = other RNA
taccgcagtttgggcgggttgctaataatattagcttatgacttctaaggcttttgcctaaaaagtaagggggaaggcgacaacccccaaagaccagccggaactatggctggcaactatctcatctttttggcatatcaaagctagggcaaaaaccccagacattcgccaaaagcccagaaccatgacattgcaagagtttcgcccagtttcttttaaagacccaagctgatttaagcggctgaaatgagat

SEQ ID NO: 283 - RNA, 253 nt, synthetic construct; identical to SEQ ID NO: 282
  source 1..253: mol_type = other RNA

SEQ ID NO: 284 - RNA, 283 nt, synthetic construct
  source 1..283: mol_type = other RNA
taccgcagtttgggcgggttgctaataatattagcttatgacttctaaggcttttgcctaaaaagtaagggggaaggcgacaacccccaaagaccagccggaactatggctggcaactatctcatctttttggcatatcaaagctagggcaaaaaccccagacattcgccaaaagcccagaaccatgacattgcaagagtttcgcccagtttcttttaaagacccaagctgatttaagcggctgaaatgagattttttaacaggtccgccaaaatcgcttctg

SEQ ID NO: 285 - RNA, 283 nt, synthetic construct; identical to SEQ ID NO: 284
  source 1..283: mol_type = other RNA

SEQ ID NO: 286 - RNA, 100 nt, synthetic construct
  source 1..100: mol_type = other RNA
ataagatggactatatttataaaccaggaaacatagttattacaattgcattgtaggtttcaacctgatacccactaaagcgtgttagtgggtatttttt

SEQ ID NO: 287 - RNA, 100 nt, synthetic construct; identical to SEQ ID NO: 286
  source 1..100: mol_type = other RNA

SEQ ID NO: 288 - RNA, 120 nt, synthetic construct
  source 1..120: mol_type = other RNA
cttgacagtaaacaaaaaataagatggactatatttataaaccaggaaacatagttattacaattgcattgtaggtttcaacctgatacccactaaagcgtgttagtgggtattttttat

SEQ ID NO: 289 - RNA, 120 nt, synthetic construct; identical to SEQ ID NO: 288
  source 1..120: mol_type = other RNA

SEQ ID NO: 290 - RNA, 161 nt, synthetic construct
  source 1..161: mol_type = other RNA
agccgcacggaacctgagccgatggcgtagcccttggacctatatggaacgcggcataagccctgcgagttcgcaagagcccaaggcggcatgacaagcctctttcaggcgacagagtcttttggagtgtcgaggctccctgcattccttgggagcctccc

SEQ ID NO: 291 - RNA, 161 nt, synthetic construct; identical to SEQ ID NO: 290
  source 1..161: mol_type = other RNA

SEQ ID NO: 292 - RNA, 93 nt, synthetic construct
  source 1..93: mol_type = other RNA
agccgcacggaacctgagccgatggcgtagcccttggacctatatggaacgcggcataagccctgcgagttcgcaagagcccaaggcggcatg

SEQ ID NO: 293 - RNA, 93 nt, synthetic construct; identical to SEQ ID NO: 292
  source 1..93: mol_type = other RNA

SEQ ID NO: 294 - RNA, 143 nt, synthetic construct
  source 1..143: mol_type = other RNA
gttccgtctcgactatgccgtaccactagaccgagcctacacggcacgcggtcatagcgttaaccaaggcgtggtgacaagcctctttcaggcgtcggacacttaagagcgttaggcgggcggtccctaagccgcccgccccc

SEQ ID NO: 295 - RNA, 143 nt, synthetic construct; identical to SEQ ID NO: 294
  source 1..143: mol_type = other RNA

SEQ ID NO: 296 - RNA, 173 nt, synthetic construct
  source 1..173: mol_type = other RNA
gttccgtctcgactatgccgtaccactagaccgagcctacacggcacgcggtcatagcgttaaccaaggcgtggtgacaagcctctttcaggcgtcggacacttaagagcgttaggcgggcggtccctaagccgcccgcccccttatttgcacgttttccccgaaccccgtaa

SEQ ID NO: 297 - RNA, 173 nt, synthetic construct; identical to SEQ ID NO: 296
  source 1..173: mol_type = other RNA

SEQ ID NO: 298 - RNA, 114 nt, synthetic construct
  source 1..114: mol_type = other RNA
gttccgtctcgactatgccgtaccactagaccgagcctacacggcacgcggtcatagcgttaaccaaggcgtggtgacaagcctctttcaggcgtcggacacttaagagcgtta

SEQ ID NO: 299 - RNA, 114 nt, synthetic construct; identical to SEQ ID NO: 298
  source 1..114: mol_type = other RNA

SEQ ID NO: 300 - RNA, 172 nt, synthetic construct
  source 1..172: mol_type = other RNA
tttcgttcgaggccgggagcaacggaccgctagcccaagggcaaccgcggccgcccggtcgaaggccgacctgtacggcctgaaggttgagaaggcacgaataagcggaaaaactcgtttcccttcgtgttcgctcaccgagcagacgtcgcaaaggcgacctgcttacagg

SEQ ID NO: 301 - RNA, 168 nt, synthetic construct
  source 1..168: mol_type = other RNA
gagaggggtattctgttgatgggcgaatcggtaaaagcaataaaattaaagatactggatatgtttttagaccccgaatgcacaaagcaggatgataactggcgcaaagatttgtctactatgtccagattctgcgctgaagccttgcgtaagcgcgtggattgaaac

SEQ ID NO: 302 - RNA, 168 nt, synthetic construct
  source 1..168: mol_type = other RNA
gagaggggtattctgttgatgggcgaatcggtaaaagcaataaaattaaagatactggatatgtttttagaccccgaatgcacaaagcaggatgataactggcgcaaagatttgtctactatgtccagattctgcgctgaagccttgcgaaagcgcgtggattgaaac

SEQ ID NO: 303 - RNA, 193 nt, synthetic construct
  source 1..193: mol_type = other RNA
gagaggggtattctgttgatgggcgaatcggtaaaagcaataaaattaaagatactggatatgtttttagaccccgaatgcacaaagcaggatgataactggcgcaaagatttgtctactatgtccagattctgcgctgaagcggggaatatgtgtctgcgcgacctgcttgcgtaagcgcgtggattgaaac

SEQ ID NO: 304 - RNA, 192 nt, synthetic construct
  source 1..192: mol_type = other RNA
gagaggggtattctgttgatgggcgaatcggtaaaagcaataaaattaaagatactggatatgtttttagaccccgaatgcacaaagcaggatgataactggcgcaaagatttgtctactatgtccagattctgcgctgaagcggggaatatgtgtctgcgcgaaaagttgcgtaagcgcgtggattgaaac

SEQ ID NO: 305 - RNA, 174 nt, synthetic construct
  source 1..174: mol_type = other RNA
tttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtcttgcgtaagcgcgtggattgaaac

SEQ ID NO: 306 - RNA, 174 nt, synthetic construct
  source 1..174: mol_type = other RNA
tttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtcttgcgaaagcgcgtggattgaaac

SEQ ID NO: 307 - RNA, 146 nt, synthetic construct
  source 1..146: mol_type = other RNA
tttactctgtttcgcgcgccagggcagttaggtgccctaaaagagcgaagtggccgaaaggaaaggctaacgcttctctaacgctacggcgaccttggcgaaatgccatcaataccacgcgcttgcgtaagcgcgtggattgaaac

SEQ ID NO: 308 - RNA, 146 nt, synthetic construct
  source 1..146: mol_type = other RNA
tttactctgtttcgcgcgccagggcagttaggtgccctaaaagagcgaagtggccgaaaggaaaggctaacgcttctctaacgctacggcgaccttggcgaaatgccatcaataccacgcgcttaaagaagcgcgtggattgaaac

SEQ ID NO: 309 - RNA, 71 nt, synthetic construct
  source 1..71: mol_type = other RNA
gtcgctatcagcggcaatgcaatttcctccgcctttggaaatttacgttctatgactttgaggtatggcgg

SEQ ID NO: 310 - RNA, 68 nt, synthetic construct
  source 1..68: mol_type = other RNA
gtcgctatcagcggcaatgcaatttcctccgcctttggaaaaagttctatgactttgaggtatggcgg

SEQ ID NO: 311 - RNA, 101 nt, synthetic construct
  source 1..101: mol_type = other RNA
gtcgctatcagcggcaatgcaatttcctccgcctttggaaatttacgttctatgactttgcgagagctgcggtgccccgttgcggccgggaggtatggcgg

SEQ ID NO: 312 - RNA, 101 nt, synthetic construct
  source 1..101: mol_type = other RNA
gtcgctatcagcggcaatgcaatttcctccgcctttggaaatttacgttctatgactttgcgagagctgcggaaagccgttgcggccgggaggtatggcgg

SEQ ID NO: 313 - RNA, 134 nt, synthetic construct
  source 1..134: mol_type = other RNA
atacgtcgcgacattccagcatgtaacccgtcgggaggattgccccatgaaccgtatttaccaaggtcggattaccggcattcttgattccaaggaggacgagcggggccaccctcccccaccaggtatggcgg

SEQ ID NO: 314 - RNA, 134 nt, synthetic construct
  source 1..134: mol_type = other RNA
atacgtcgcgacattccagcatgtaacccgtcgggaggattgccccatgaaccgtatttaccaaggtcggattaccggcattcttgattccaaggaggacgagcggggccaccctcccccaaagggtatggcgg

SEQ ID NO: 315 - RNA, 164 nt, synthetic construct
  source 1..164: mol_type = other RNA
atacgtcgcgacattccagcatgtaacccgtcgggaggattgccccatgaaccgtatttaccaaggtcggattaccggcattcttgattccaaggaggacgagcggggccaccctcccccacccgatccgaaacacaatccattctggcggcaaggtatggcgg

SEQ ID NO: 316 - RNA, 160 nt, synthetic construct
  source 1..160: mol_type = other RNA
atacgtcgcgacattccagcatgtaacccgtcgggaggattgccccatgaaccgtatttaccaaggtcggattaccggcattcttgattccaaggaggacgagcggggaaagccccacccgatccgaaacacaatccattctggcggcaaggtatggcgg

SEQ ID NO: 317 - RNA, 128 nt, synthetic construct
  source 1..128: mol_type = other RNA
cttgacatggcttaagaatgcgccatgtatgaaggagaagtaacctgcttccttcctcgctgtcaccgccttcgtcagcgatttcatcggcggaacataataattttacttttttctaggtatggcgg

SEQ ID NO: 318 - RNA, 126 nt, synthetic construct
  source 1..126: mol_type = other RNA
cttgacatggcttaagaatgcgccatgtatgaaggagaagtaacctgcttccttcctcgctgtcaccgccttcgtcagcgatttcatcggcggaacataataattttacttaaagaggtatggcgg

SEQ ID NO: 319 - RNA, 105 nt, synthetic construct
  source 1..105: mol_type = other RNA
ggtatatccgttttgttctgctctctatcgcattcggggggcaatgcgtcaaagattagtgacgtgtttgcgaaatagacgtctattgatattactgaatggcgg

SEQ ID NO: 320 - RNA, 105 nt, synthetic construct
  source 1..105: mol_type = other RNA
ggtatatccgttttgttctgctctctatcgcattcggggggcaatgcgtcaaagattagtgacgtgtttgcgaaatagaaagctattgatattactgaatggcgg

SEQ ID NO: 321 - RNA, 99 nt, synthetic construct
  source 1..99: mol_type = other RNA
aaagtgtacgtctttttgtacaacctcttcggccttctggctacgggtgccctggatcacgccgctgtagaagggcgtccattcacggctgacggaaac

SEQ ID NO: 322 - RNA, 98 nt, synthetic construct
  source 1..98: mol_type = other RNA
aaagtgtacgtctttttgtacaacctcttcggccttctggctacgggtgccctggatcacgccgctaaagagggcgtccattcacggctgacggaaac

SEQ ID NO: 323 - RNA, 129 nt, synthetic construct
  source 1..129: mol_type = other RNA
aaagtgtacgtctttttgtacaacctcttcggccttctggctacgggtgccctggatcacgcctttgggacctgggaaaccacaacctttcccgctgtagaagggcgtccattcacggctgacggaaac

SEQ ID NO: 324 - RNA, 125 nt, synthetic construct
  source 1..125: mol_type = other RNA
aaagtgtacgtctttttgtacaacctcttcggccttctggctacgggtgccctggatcacgcctttgggacctgggaaaaaagtttcccgctgtagaagggcgtccattcacggctgacggaaac

SEQ ID NO: 325 - RNA, 80 nt, synthetic construct
  source 1..80: mol_type = other RNA
aaatctcggtcgatggaaatgttgccctctttggtgatgtgatagctgtagaagggcgtccattcacggctgacggaaac

SEQ ID NO: 326 - RNA, 75 nt, synthetic construct
  source 1..75: mol_type = other RNA
aaatctcggtcgatggaaatgttgccctctttggtaaaggctgtagaagggcgtccattcacggctgacggaaac

SEQ ID NO: 327 - RNA, 89 nt, synthetic construct
  source 1..89: mol_type = other RNA
cgattccacctaactgtctgacaggaaactgcttcgccgcattgatgcttcgagctgtagaagggcgtccattcacggctgacggaaac

SEQ ID NO: 328 - RNA, 89 nt, synthetic construct
  source 1..89: mol_type = other RNA
cgattccacctaactgtctgacaggaaactgcttcgccgcattgatgcttcgagctgtagaaggaaagccattcacggctgacggaaac

SEQ ID NO: 329 - RNA, 93 nt, synthetic construct
  source 1..93: mol_type = other RNA
aaattatcggccgggacggctcctttgccccagggcaggtgagcccgtagtttcgcggctgtagaagggcgtccattcacggctgacggaaac

SEQ ID NO: 330 - RNA, 90 nt, synthetic construct
  source 1..90: mol_type = other RNA
aaattatcggccgggacggctcctttgccccagggcaggtgagcccgtagtttcgcggctaaagggcgtccattcacggctgacggaaac

SEQ ID NO: 331 - RNA, 123 nt, synthetic construct
  source 1..123: mol_type = other RNA
aaattatcggccgggacggctcctttgccccagggcaggtgagcccgtagtttcgcggccgggtactggacggataggcgaatgacggctgtagaagggcgtccattcacggctgacggaaac

SEQ ID NO: 332 - RNA, 121 nt, synthetic construct
  source 1..121: mol_type = other RNA
aaattatcggccgggacggctcctttgccccagggcaggtgagcccgtagtttcgcggccgggtactggacggataggcgaatgacggctaaaggggcgtccattcacggctgacggaaac

SEQ ID NO: 333 - RNA, 138 nt, synthetic construct
  source 1..138: mol_type = other RNA
gttcgatctctttcaacaatggttgaaggaaattatcggccgggacggctcctttgccccagggcaggtgagcccgtagtttcgcggccgggtactggacgggctgtagaagggcgtccattcacggctgacggaaac

SEQ ID NO: 334 - RNA, 134 nt, synthetic construct
  source 1..134: mol_type = other RNA
gttcgatctctttcaacaatggttgaaggaaattatcggccgggacggctcctttgccccagggcaggtgagcccgtagtttcgcggccggaaagcgggctgtagaagggcgtccattcacggctgacggaaac

SEQ ID NO: 335 - RNA, 96 nt, synthetic construct
  source 1..96: mol_type = other RNA
gttcccagccttccaggaatggttggtcgggatcagacgccgggttggtgagccagcttgttgcaacggcttccggagtgctggtgggaatgaaac

SEQ ID NO: 336 - RNA, 95 nt, synthetic construct
  source 1..95: mol_type = other RNA
gttcccagccttccaggaatggttggtcgggatcagacgccgggttggtgagccagcaaaggcaacggcttccggagtgctggtgggaatgaaac

SEQ ID NO: 337 - RNA, 126 nt, synthetic construct
  source 1..126: mol_type = other RNA
gttcccagccttccaggaatggttggtcgggatcagacgccgggttggtgagccagctttaatcggccggtgagccaggtagtttcatcgttgcaacggcttccggagtgctggtgggaatgaaac

SEQ ID NO: 338 - RNA, 124 nt, synthetic construct
  source 1..124: mol_type = other RNA
gttcccagccttccaggaatggttggtcgggatcagacgccgggttggtgagccagctaaagggccggtgagccaggtagtttcatcgttgcaacggcttccggagtgctggtgggaatgaaac

SEQ ID NO: 339 - RNA, 137 nt, synthetic construct
  source 1..137: mol_type = other RNA
agcttaggctgaagggaggcgctgcattctgagtcgggtccggagctgttgcaacggcttccggagtgctggtgggaatgaaacatgatctcttttctccgttgcaacggcttccggagtgctggtgggaatgaaac

SEQ ID NO: 340 - RNA, 132 nt, synthetic construct
  source 1..132: mol_type = other RNA
agcttaggctgaagggaggcgctgcattctgagtcgggtccggagctgttgcaacggcttccggagtgctggtaaagacatgatctcttttctccgttgcaacggcttccggagtgctggtgggaatgaaac

SEQ ID NO: 341 - RNA, 171 nt, synthetic construct
  source 1..171: mol_type = other RNA
gttcgatctctttcaacagtggttgaaggagcttatcggccgggacggttcctctgccttcgggcaagggaaccagtagtttcgcggccgggtactgggcggataggcgaatgacggcagcctcacggctggcgcgctgtagaggggcgtccattcacggctgacggaaac

SEQ ID NO: 342 - RNA, 171 nt, synthetic construct
  source 1..171: mol_type = other RNA
gttcgatctctttcaacagtggttgaaggagcttatcggccgggacggttcctctgccttcgggcaagggaaccagtagtttcgcggccgggtactgggcggataggcgaatgacggcagccaaagggctggcgcgctgtagaggggcgtccattcacggctgacggaaac

SEQ ID NO: 343 - RNA, 201 nt, synthetic construct
  source 1..201: mol_type = other RNA
gttcgatctctttcaacagtggttgaaggagcttatcggccgggacggttcctctgccttcgggcaagggaaccagtagtttcgcggccgggtactgggcggataggcgaatgacggcagcctcacggctggcgcgtaaccgaccatcccctggtccgaacagccgctgtagaggggcgtccattcacggctgacggaaac

SEQ ID NO: 344 - RNA, 200 nt, synthetic construct
  source 1..200: mol_type = other RNA
gttcgatctctttcaacagtggttgaaggagcttatcggccgggacggttcctctgccttcgggcaagggaaccagtagtttcgcggccgggtactgggcggataggcgaatgacggcagcctcacggctggcgcgtaaccgaccatcccctggtccgaacagccgctgtagaggaaagccattcacggctgacggaaac

SEQ ID NO: 345 - RNA, 136 nt, synthetic construct
  source 1..136: mol_type = other RNA
gttcgatctctttcaacagtggttgaaggagcttatcggccgggacggttcctctgccttcgggcaagggaaccagtagtttcgcggccgggtactgggcgctgtagaggggcgtccattcacggctgacggaaac

SEQ ID NO: 346 - RNA, 133 nt, synthetic construct
  source 1..133: mol_type = other RNA
gttcgatctctttcaacagtggttgaaggagcttatcggccgggacggttcctctgccttcgggcaagggaaccagtagtttcgcggccgggtactgggcgctaaagggcgtccattcacggctgacggaaac

SEQ ID NO: 347 - RNA, 134 nt, synthetic construct
  source 1..134: mol_type = other RNA
gttcgatctctttcagcaatggttgaaggagcttatcggccgggacggttctgttgccctcgggcaggggaaccagtagtttcgcggccgggtactgggctgtataagggcgtccattcacggctgacggaaac

SEQ ID NO: 348 - RNA, 132 nt, synthetic construct
  source 1..132: mol_type = other RNA
gttcgatctctttcagcaatggttgaaggagcttatcggccgggacggttctgttgccctcgggcaggggaaccagtagtttcgcggccgggtactgggctgtaaaggcgtccattcacggctgacggaaac

SEQ ID NO: 349 - RNA, 174 nt, synthetic construct
  source 1..174: mol_type = other RNA
gaagttcgatctctttcaacagtggttgaaggagcttatcggccgggacggttccgctgccccagggtagtggaaccagtagtttcgcggccgggtactgggcggataggcgaatgacggcatcctcacggttgccgcgctgtagaggggcgtccattcacggctgacggaaac

SEQ ID NO: 350 - RNA, 173 nt, synthetic construct
  source 1..173: mol_type = other RNA
gaagttcgatctctttcaacagtggttgaaggagcttatcggccgggacggttccgctgccccagggtagtggaaccagtagtttcgcggccgggtactgggcggataggcgaaagcggcatcctcacggttgccgcgctgtagaggggcgtccattcacggctgacggaaac

SEQ ID NO: 351 - RNA, 204 nt, synthetic construct
  source 1..204: mol_type = other RNA
gaagttcgatctctttcaacagtggttgaaggagcttatcggccgggacggttccgctgccccagggtagtggaaccagtagtttcgcggccgggtactgggcggataggcgaatgacggcatcctcacggttgccgcgtaaccgaccatcccctggtccgaacagccgctgtagaggggcgtccattcacggctgacggaaac

SEQ ID NO: 352 - RNA, 203 nt, synthetic construct
  source 1..203: mol_type = other RNA
gaagttcgatctctttcaacagtggttgaaggagcttatcggccgggacggttccgctgccccagggtagtggaaccagtagtttcgcggccgggtactgggcggataggcgaatgacggcatcctcacggttgccgcgtaaccgaccatcccctggtccgaacagccgctgtagaggaaagccattcacggctgacggaaac

SEQ ID NO: 353 - RNA, 139 nt, synthetic construct
  source 1..139: mol_type = other RNA
gaagttcgatctctttcaacagtggttgaaggagcttatcggccgggacggttccgctgccccagggtagtggaaccagtagtttcgcggccgggtactgggcgctgtagaggggcgtccattcacggctgacggaaac

SEQ ID NO: 354 - RNA, 136 nt, synthetic construct
  source 1..136: mol_type = other RNA
gaagttcgatctctttcaacagtggttgaaggagcttatcggccgggacggttccgctgccccagggtagtggaaccagtagtttcgcggccgggtactgggcgctaaagggcgtccattcacggctgacggaaac

SEQ ID NO: 355 - RNA, 146 nt, synthetic construct
  source 1..146: mol_type = other RNA
gttcgatctctttcaactgtggttgaaggagcttatcggccgggacggttccattgcctcagggcaggggaaccagtagtttcgcggccgggtactgggcggataggcgaatgatggcatcctcacggttgccgcctgacggaaac

SEQ ID NO: 356 - RNA, 147 nt, synthetic construct
  source 1..147: mol_type = other RNA
gttcgatctctttcaactgtggttgaaggagcttatcggccgggacggttccattgcctcagggcaggggaaccagtagtttcgcggccgggtactgggcggataggcgaatgatggaaagcctcacggttgccgcctgacggaaac

SEQ ID NO: 357 - RNA, 176 nt, synthetic construct
  source 1..176: mol_type = other RNA
gttcgatctctttcaactgtggttgaaggagcttatcggccgggacggttccattgcctcagggcaggggaaccagtagtttcgcggccgggtactgggcggataggcgaatgatggcatcctcacggttgccgcgtaaccgaccatcccctggtccgaacagccctgacggaaac

SEQ ID NO: 358 - RNA, 176 nt, synthetic construct
  source 1..176: mol_type = other RNA
gttcgatctctttcaactgtggttgaaggagcttatcggccgggacggttccattgcctcagggcaggggaaccagtagtttcgcggccgggtactgggcggataggcgaatgatggcatcctcacggttgccgcgtaaccgaccatcccctggtccgaacagcccaaagggaaac

SEQ ID NO: 359 - RNA, 163 nt, synthetic construct
  source 1..163: mol_type = other RNA
atatctcaagccatgttcaaaacggcgcgtttcaaggttcacaatccgtcacggcacaagagcactatgctctggtatgccatgacccgctatcacgagactttgaaggacgtactcgaaaagacactggcgattccagatctgctagaacactgacggaaac

SEQ ID NO: 360 - RNA, 163 nt, synthetic construct
  source 1..163: mol_type = other RNA
atatctcaagccatgttcaaaacggcgcgtttcaaggttcacaatccgtcacggcacaagagcactatgctctggtatgccatgacccgctatcacgagactttgaaggacgtactcgaaaagacactggcgattccagatctgctagaaagctgacggaaac

SEQ ID NO: 361 - RNA, 173 nt, synthetic construct
  source 1..173: mol_type = other RNA
atatctcaagccatgttcaaaacggcgcgtttcaaggttcacaatccgtcacggcacaagagcactatgctctggtatgccatgacccgctatcacgagactttgaaggacgtactcgaaaagacactggcgattccagatctgctagaacaaatctcagaactgacggaaac

SEQ ID NO: 362 - RNA, 172 nt, synthetic construct
  source 1..172: mol_type = other RNA
atatctcaagccatgttcaaaacggcgcgtttcaaggttcacaatccgtcacggcacaagagcactatgctctggtatgccatgacccgctatcacgagactttgaaggacgtactcgaaaagacactggcgattccagatctgctagaaaagtctcagaactgacggaaac

SEQ ID NO: 363 - RNA, 111 nt, synthetic construct
  source 1..111: mol_type = other RNA
gttcgatctctttcaactgtggttgaaggagcttatcggccgggacggttccattgcctcagggcaggggaaccagtagtttcgcggccgggtactgggcctgacggaaac

SEQ ID NO: 364 - RNA, 110 nt, synthetic construct
  source 1..110: mol_type = other RNA
gttcgatctctttcaactgtggttgaaggagcttatcggccgggacggttccattgcctcagggcaggggaaccagtagtttcgcggccgggtaaaggcctgacggaaac

SEQ ID NO: 365 - RNA, 107 nt, synthetic construct
  source 1..107: mol_type = other RNA
caaattcggtgcaagggcgaagcccaaccggtagactcatatcttccggtaacgcacctcggctgtttagacctgtagaagggcgtccattcacggctgacggaaac

SEQ ID NO: 366 - RNA, 104 nt, synthetic construct
  source 1..104: mol_type = other RNA
caaattcggtgcaagggcgaagcccaaccggtagactcatatcttccggtaacgcacctcggctgtttagacctaaagggcgtccattcacggctgacggaaac

SEQ ID NO: 367 - RNA, 137 nt, synthetic construct
  source 1..137: mol_type = other RNA
caaattcggtgcaagggcgaagcccaaccggtagactcatatcttccggtaacgcacctcggctgtttagaccagaggatggtcggttacgcggcaaccgtgatgtagaagggcgtccattcacggctgacggaaac

SEQ ID NO: 368 - RNA, 137 nt, synthetic construct
  source 1..137: mol_type = other RNA
caaattcggtgcaagggcgaagcccaaccggtagactcatatcttccggtaacgcacctcggctgtttagaccagaggatggtcggttacgcggcaaccgtgatgtagaaggaaagccattcacggctgacggaaac

SEQ ID NO: 369 - RNA, 134 nt, synthetic construct
  source 1..134: mol_type = other RNA
gttcgatatctttcaacagtggttgaaggagcttatcggccgggacggttcctctgccccagggcagttgaacccgtagtttcgcggccgggtactgggctgtagaagggcgtccattcacggctgacggaaac

SEQ ID NO: 370 - RNA, 132 nt, synthetic construct
  source 1..132: mol_type = other RNA
gttcgatatctttcaacagtggttgaaggagcttatcggccgggacggttcctctgccccagggcagttgaacccgtagtttcgcggccgggtactgggctgtaaaggcgtccattcacggctgacggaaac

SEQ ID NO: 371 - RNA, 96 nt, synthetic construct
  source 1..96: mol_type = other RNA
attgatgaggctgtctgacttgtgcaggctcattattgataggattgatgaggatgacttaaaacgcgaaacctcatcaatcctatcaataatgag

SEQ ID NO: 372 - RNA, 82 nt, synthetic construct
  source 1..82: mol_type = other RNA
attgatgaggctgtctgacttgtgcaggctcattattgataggattgatgaggaaagcctcatcaatcctatcaataatgag

SEQ ID NO: 373 - RNA, 126 nt, synthetic construct
  source 1..126: mol_type = other RNA
attgatgaggctgtctgacttgtgcaggctcattattgataggattgatgaggatgacttaaaacgcgaaactcattattgataggattgatgaggattttcctcatcaatcctatcaataatgag

SEQ ID NO: 374 - RNA, 127 nt, synthetic construct
  source 1..127: mol_type = other RNA
attgatgaggctgtctgacttgtgcaggctcattattgataggattgatgaggatgacttaaaacgcgaaactcattattgataggattgatgaggaaaagtcctcatcaatcctatcaataatgag

SEQ ID NO: 375 - RNA, 114 nt, synthetic construct
  source 1..114: mol_type = other RNA
acaatccctcttgagcattggatgcagattcttctgtctccgtacctcgctcataagggcgctgtcgggatagcagtgcctatcaaaaccctcatcaatcctatcaataatgag

SEQ ID NO: 376 - RNA, 104 nt, synthetic construct
  source 1..104: mol_type = other RNA
acaatccctcttgagcattggatgcagattcttctgtctccgtacctcgctcataagggcgctgtcgggatagcagtgcctatcaaaaccctcataaagatgag

SEQ ID NO: 377 - RNA, 144 nt, synthetic construct
  source 1..144: mol_type = other RNA
acaatccctcttgagcattggatgcagattcttctgtctccgtacctcgctcataagggcgctgtcgggatagcagtgcctatcaaaactatcccactataatgcttgtttctttacctcctcatcaatcctatcaataatgag

SEQ ID NO: 378 - RNA, 134 nt, synthetic construct
  source 1..134: mol_type = other RNA
acaatccctcttgagcattggatgcagattcttctgtctccgtacctcgctcataagggcgctgtcgggatagcagtgcctatcaaaactatcccactataatgcttgtttctttacctcctcataaagatgag

SEQ ID NO: 379 - RNA, 63 nt, synthetic construct
  source 1..63: mol_type = other RNA
tattgataggattgatgaggtcgagcatcgaaaagattcctcatcaatcctatcaataatgag

SEQ ID NO: 380 - RNA, 64 nt, synthetic construct
  source 1..64: mol_type = other RNA
tattgataggattgatgaggtcgaaaagtcgaaaagattcctcatcaatcctatcaataatgag

SEQ ID NO: 381 - RNA, 93 nt, synthetic construct
  source 1..93: mol_type = other RNA
tattgataggattgatgaggtcgagcatcgaaaagattctcattattgataggattgatgaggcttaacctcatcaatcctatcaataatgag

SEQ ID NO: 382 - RNA, 92 nt, synthetic construct
  source 1..92: mol_type = other RNA
tattgataggattgatgaggtcgagcatcgaaaagattctcattattgataggattgatgaggaaagcctcatcaatcctatcaataatgag

SEQ ID NO: 383 - RNA, 124 nt, synthetic construct
  source 1..124: mol_type = other RNA
ctcaaactccttccggctttcggaccgtctgcttgcagacctccttaccttggccggagggacatcgccgggggtcgatgcctgtcacttacccccattccttcaaaaccctgtcacatctgga

SEQ ID NO: 384 - RNA, 108 nt, synthetic construct
  source 1..108: mol_type = other RNA
ctcaaactccttccggctttcggaccgtctgcttgcagacctccttaccttggccggagggacatcgccgggggtcgatgcctgtcacttacccccattccaaaggga

SEQ ID NO: 385 - RNA, 154 nt, synthetic construct
  source 1..154: mol_type = other RNA
ctcaaactccttccggctttcggaccgtctgcttgcagacctccttaccttggccggagggacatcgccgggggtcgatgcctgtcacttacccccatttcacccacttcttggcttggtttctccgggccttcaaaaccctgtcacatctgga

SEQ ID NO: 386 - RNA, 152 nt, synthetic construct
  source 1..152: mol_type = other RNA
ctcaaactccttccggctttcggaccgtctgcttgcagacctccttaccttggccggagggacatcgccgggggtcgatgcctgtcacttacccccatttcacccacttcttggcttggtttctccgggccttcaaaaccctgaaagctgga

SEQ ID NO: 387 - RNA, 131 nt, synthetic construct
  source 1..131: mol_type = other RNA
gcactgacacgcccagaaatgacagggttctaatcggctgatcccgccttgaattgaccagaggccgtaaggattcaaaaggacttcaatgcggcctcatgtctctccttcaaaaccctgtcacatctgga

SEQ ID NO: 388 - RNA, 130 nt, synthetic construct
  source 1..130: mol_type = other RNA
gcactgacacgcccagaaatgacagggttctaatcggctgatcccgccttgaattgaccagaggccgtaaggattcaaaggacttcaatgcggcctcatgtctctccttcaaaaccctgtcacatctgga

SEQ ID NO: 389 - RNA, 161 nt, synthetic construct
  source 1..161: mol_type = other RNA
gcactgacacgcccagaaatgacagggttctaatcggctgatcccgccttgaattgaccagaggccgtaaggattcaaaaggacttcaatgcggcctcatgtctctctccggttcgaccggctacgggaaggttctccttcaaaaccctgtcacatctgga

SEQ ID NO: 390 - RNA, 161 nt, synthetic construct
  source 1..161: mol_type = other RNA
gcactgacacgcccagaaatgacagggttctaatcggctgatcccgccttgaattgaccagaggccgtaaggattcaaaaggacttcaatgcggcctcatgtctctctccggttcgaccggctacgggaaggaaagccttcaaaaccctgtcacatctgga

SEQ ID NO: 391 - RNA, 133 nt, synthetic construct
  source 1..133: mol_type = other RNA
tcagaaactcccgacttcctcaggatgaagttgattctttatccataccttggtgccgggacgccgattgaggaatgggcggcgccttccaaattctcattcctcactccttataaacccttccaataatggg

SEQ ID NO: 392 - RNA, 127 nt, synthetic construct
  source 1..127: mol_type = other RNA
tcagaaactcccgacttcctcaggatgaagttgattctttatccataccttggtgccgggacgccgattgaggaatgggcggcgccttccaaattctcattcctcactccttataaacccaaagggg

SEQ ID NO: 393 - RNA, 163 nt, synthetic construct
  source 1..163: mol_type = other RNA
tcagaaactcccgacttcctcaggatgaagttgattctttatccataccttggtgccgggacgccgattgaggaatgggcggcgccttccaaattctcattcctcactttcctcctgcgaaacttcatcgaagcaaatccttataaacccttccaataatggg

SEQ ID NO: 394 - RNA, 157 nt, synthetic construct
  source 1..157: mol_type = other RNA
tcagaaactcccgacttcctcaggatgaagttgattctttatccataccttggtgccgggacgccgattgaggaatgggcggcgccttccaaattctcattcctcactttcctcctgcgaaacttcatcgaagcaaatccttataaacccaaagggg

SEQ ID NO: 395 - RNA, 133 nt, synthetic construct
  source 1..133: mol_type = other RNA
cagaaactcccgacttcctcaggatgaagttgattctttatccataccttggtgccgggacgccgattgaggaatgggcggcgccttccaaattctcattcctcacttccttataaacccttccaataatggg

SEQ ID NO: 396 - RNA, 127 nt, synthetic construct
  source 1..127: mol_type = other RNA
cagaaactcccgacttcctcaggatgaagttgattctttatccataccttggtgccgggacgccgattgaggaatgggcggcgccttccaaattctcattcctcacttccttataaacccaaagggg

SEQ ID NO: 397 - RNA, 163 nt, synthetic construct
  source 1..163: mol_type = other RNA
cagaaactcccgacttcctcaggatgaagttgattctttatccataccttggtgccgggacgccgattgaggaatgggcggcgccttccaaattctcattcctcactttcctcctgcgaaacttcatcgaagcaaattccttataaacccttccaataatggg

SEQ ID NO: 398 - RNA, 157 nt, synthetic construct
  source 1..157: mol_type = other RNA
cagaaactcccgacttcctcaggatgaagttgattctttatccataccttggtgccgggacgccgattgaggaatgggcggcgccttccaaattctcattcctcactttcctcctgcgaaacttcatcgaagcaaattccttataaacccaaagggg

SEQ ID NO: 399 - RNA, 153 nt, synthetic construct
  source 1..153: mol_type = other RNA
actagttaaagaggagaatagattatggaactaacagaagaacagcaccaagatttcatttggacttggccctcggtccactccagaggtaaagccagtctctggttaccttattttcaaggtttagagaagttatcttatgacgaactgagg

SEQ ID NO: 400 - RNA, 151 nt, synthetic construct
  source 1..151: mol_type = other RNA
actagttaaagaggagaatagattatggaactaacagaagaacagcaccaagatttcatttggacttggccctcggtccactccagaggtaaagccagtctctggttaccttaaagaaggtttagagaagttatcttatgacgaactgagg

SEQ ID NO: 401 - RNA, 183 nt, synthetic construct
  source 1..183: mol_type = other RNA
actagttaaagaggagaatagattatggaactaacagaagaacagcaccaagatttcatttggacttggccctcggtccactccagaggtaaagccagtctctggttaccttattttcaaggtttagagaagttgggtcgcggtaagccttaccagtataagttatcttatgacgaactgagg

SEQ ID NO: 402 - RNA, 184 nt, synthetic construct
  source 1..184: mol_type = other RNA
actagttaaagaggagaatagattatggaactaacagaagaacagcaccaagatttcatttggacttggccctcggtccactccagaggtaaagccagtctctggttaccttattttcaaggtttagagaagttgggtcgcggtaaaaagttaccagtataagttatcttatgacgaactgagg

SEQ ID NO: 403 - RNA, 83 nt, synthetic construct
  source 1..83: mol_type = other RNA
agttcgaaggtacttgttgattgatcgggtcaaaccaataatcagcctaggaacaattttgaatatcttatgacgaactgagg

SEQ ID NO: 404 - RNA, 79 nt, synthetic construct
  source 1..79: mol_type = other RNA
agttcgaaggtacttgttgattgatcgggtcaaaccaataatcagcctaggaacaattttgaatatcttaaaagtgagg

SEQ ID NO: 405 - RNA, 82 nt, synthetic construct
  source 1..82: mol_type = other RNA
atggggtttggtatatcttccgctccattatcttggtgtaaagtgtatgagcctgttcacggttacgtaggtgatatggaag

SEQ ID NO: 406 - RNA, 82 nt, synthetic construct
  source 1..82: mol_type = other RNA
atggggtttggtatatcttccgctccattatcttggtgtaaagtgtatgagcctgttcacggttacaaaggtgatatggaag

SEQ ID NO: 407 - RNA, 126 nt, synthetic construct
  source 1..126: mol_type = other RNA
atggggtttggtatatcttccgctccattatcttggtgtaaagtgtatgagcctatcttggccttgctctactctaggttagaaagtgctctcaacctgttcacggttacgtaggtgatatggaag

SEQ ID NO: 408 - RNA, 126 nt, synthetic construct
  source 1..126: mol_type = other RNA
atggggtttggtatatcttccgctccattatcttggtgtaaagtgtatgagcctatcttggccttgctctactctaggttagaaagtgctctcaacctgttcacggttacaaaggtgatatggaag

SEQ ID NO: 409 - RNA, 128 nt, synthetic construct
  source 1..128: mol_type = other RNA
agaagggacagaaactcaaagaccatgaacagttttcggcgtggtacgccgatctgaaaaacgggggtggaggccatgcagatggtccttagttacagcctccgtggacttgtggggtgactgtgaca

SEQ ID NO: 410 - RNA, 128 nt, synthetic construct
  source 1..128: mol_type = other RNA
agaagggacagaaactcaaagaccatgaacagttttcggcgtggtacgccgatctgaaaaacgggggtggaggccataaagatggtccttagttacagcctccgtggacttgtggggtgactgtgaca

SEQ ID NO: 411 - RNA, 158 nt, synthetic construct
  source 1..158: mol_type = other RNA
agaagggacagaaactcaaagaccatgaacagttttcggcgtggtacgccgatctgaaaaacgggggtggaggccatgcagatggtccttagttacagcctccgtggactgcgttgcgggtgtatgaccggaagtttctttgtggggtgactgtgaca

SEQ ID NO: 412 - RNA, 154 nt, synthetic construct
  source 1..154: mol_type = other RNA
agaagggacagaaactcaaagaccatgaacagttttcggcgtggtacgccgatctgaaaaacgggggtggaggccatgcagatggtccttagttacagcctccgtggactgcgttgcggaaagccggaagtttctttgtggggtgactgtgaca

SEQ ID NO: 413 - RNA, 86 nt, synthetic construct
  source 1..86: mol_type = other RNA
agaagggacagaaactcaaagaccatgaacagttttcggcgtggtacgccgatctgaaaaacgggggttgtggggtgactgtgaca

SEQ ID NO: 414 - RNA, 87 nt, synthetic construct
  source 1..87: mol_type = other RNA
agaagggacagaaactcaaagaccatgaacagttttcggcgtaaagacgccgatctgaaaaacgggggttgtggggtgactgtgaca

SEQ ID NO: 415 - RNA, 99 nt, synthetic construct
  source 1..99: mol_type = other RNA
ctacacagccggcgggacgtatcccgtcggccgcgcccgactgcacgggcgaggcagtgaaggcgtcagacgtctctccacgcgcgcgcgggatgcggg

SEQ ID NO: 416 - RNA, 98 nt, synthetic construct
  source 1..98: mol_type = other RNA
ctacacagccggcgggacgtatcccgtcggccgcgcccgactgcacgggcgaggcagtgaaaagtcagacgtctctccacgcgcgcgcgggatgcggg

SEQ ID NO: 417 - RNA, 71 nt, synthetic construct
  source 1..71: mol_type = other RNA
ctacacagccggcgggacgtatcccgtcggccgcgcccgactgcactctccacgcgcgcgcgggatgcggg

SEQ ID NO: 418 - RNA, 68 nt, synthetic construct
  source 1..68: mol_type = other RNA
ctacacagccggcgggacgtatcccgtcggccgcgcccgactgcacaaaggcgcgcgcgggatgcggg

SEQ ID NO: 419 - RNA, 115 nt, synthetic construct
  source 1..115: mol_type = other RNA
atggcaggcgctgcctagtgcgcccgtcgcggtagtaatcacgtcgagacgcaaaacgcctggggacggtgtaggtagcaaaccggatgaaccagggattcgcgtggagtgatgg

SEQ ID NO: 420 - RNA, 126 nt, synthetic construct
  source 1..126: mol_type = other RNA
atggcaggcgctgcctagtgcgcccgtcgcggtagtaatcacgtcgagacgcaaaacgcctggggacggtgtaggtagcaaaccggatgaaccaggagttcaccccacaggcgcgtggagtgatgg

SEQ ID NO: 421 - RNA, 125 nt, synthetic construct
  source 1..125: mol_type = other RNA
atggcaggcgctgcctagtgcgcccgtcgcggtagtaatcacgtcgagacgcaaaacgcctggggacggtgtaggtagcaaaccggatgaacaaaggttcaccccacaggcgcgtggagtgatgg

SEQ ID NO: 422 - RNA, 62 nt, synthetic construct
  source 1..62: mol_type = other RNA
gcagtgcccgacctgcggcgcagaacatgatcggttcaccccacaggcgcgtggagtgatgg

SEQ ID NO: 423 - RNA, 60 nt, synthetic construct
  source 1..60: mol_type = other RNA
gcagtgcccgacctgcggcgcagaacatgatcggttcaccccacaaaggtggagtgatgg

SEQ ID NO: 424 - RNA, 92 nt, synthetic construct
  source 1..92: mol_type = other RNA
gcagtgcccgacctgcggcgcagaacatgatcgggacggcaacgcagcggtcaacatccgcaagttcaccccacaggcgcgtggagtgatgg

SEQ ID NO: 425 - RNA, 90 nt, synthetic construct
  source 1..90: mol_type = other RNA
gcagtgcccgacctgcggcgcagaacatgatcgggacggcaacgcagcggtcaacatccgcaagttcaccccacaaaggtggagtgatgg

SEQ ID NO: 426 - RNA, 146 nt, synthetic construct
  source 1..146: mol_type = other RNA
atggcgaaaccacgaaccaaacaagagtacggcccgtttagcgtgcgtctgcctgttgagatgcgccagcagatcgaaacgttggcagaacaagagatgcggtcgcttcactcgatggttcaccccacgggtgcgtggagtgatgg

SEQ ID NO: 427 - RNA, 146 nt, synthetic construct
  source 1..146: mol_type = other RNA
atggcgaaaccacgaaccaaacaagagtacggcccgtttagcgtgcgtctgcctgttgagatgcgccagcagatcgaaacgttggcagaacaagagatgcggtcgcttcactcgatggttcaccccacgaaagcgtggagtgatgg

SEQ ID NO: 428 - RNA, 136 nt, synthetic construct
  source 1..136: mol_type = other RNA
gcagcgtcgcggggttttaatgggccgacttcccggcctgaagccgaggtagcagccatgtacggcgattctcgaatcgaggaaggaaacatggacgcgctcggtaaatcatgaatggatgctgattgatggaagg

SEQ ID NO: 429 - RNA, 137 nt, synthetic construct
  source 1..137: mol_type = other RNA
gcagcgtcgcggggttttaatgggccgacttcccggcctgaagccgaggtagcagccatgtacggcgattctcgaaagcgaggaaggaaacatggacgcgctcggtaaatcatgaatggatgctgattgatggaagg

SEQ ID NO: 430 - RNA, 103 nt, synthetic construct
  source 1..103: mol_type = other RNA
tcgaggaaggaaacatggacgcgctcggtaaatcgtcccgagatgggaccaatcagcatccgccttgcgacgctgccatgaatggatgctgattgatggaagg

SEQ ID NO: 431 - RNA, 103 nt, synthetic construct
  source 1..103: mol_type = other RNA
tcgaggaaggaaacatggacgcgctcggtaaatcgtcccgagatgggaccaatcagcatccgccttgcgaaagtgccatgaatggatgctgattgatggaagg

SEQ ID NO: 432 - RNA, 141 nt, synthetic construct
  source 1..141: mol_type = other RNA
aatgggccgacttcccggcctgaagccgaggtagcagccatgtacggcgattctcgaatcgaggaaggaaacatggacgcgctcggtaaatcgtcccgagatgggaccaatcagcatgaatggatgctgattgatggaagg

SEQ ID NO: 433 - RNA, 139 nt, synthetic construct
  source 1..139: mol_type = other RNA
aatgggccgacttcccggcctgaagccgaggtagcagccatgtacggcgattctcgaatcgaggaaggaaacatggacgcgctcggtaaatcgtcccgagatgggaccaatcagcataaagatgctgattgatggaagg

SEQ ID NO: 434 - RNA, 156 nt, synthetic construct
  source 1..156: mol_type = other RNA
gcagcgtcgcggggttttaatgggccgacttcccggcctgaagccgaggtagcagccatgtacggcgattctcgaatcgaggaaggaaacatggacgcgctcggtaaatcgtcccgagatgggaccaatcatgaatggatgctgattgatggaagg

SEQ ID NO: 435 - RNA, 155 nt, synthetic construct
  source 1..155: mol_type = other RNA
gcagcgtcgcggggttttaatgggccgacttcccggcctgaagccgaggtagcagccatgtacggcgattctcgaatcgaggaaggaaacatggacgcgctcggtaaatcgtcccaaaggggaccaatcatgaatggatgctgattgatggaagg

SEQ ID NO: 436 - RNA, 122 nt, synthetic construct
  source 1..122: mol_type = other RNA
gaaaccataggtagaggcgccaccaccttacatggtgccgataccgctccgttggtgcagtgtggactgtaatggtagaggcttatggtagaggtgccaccggtttacatggcgccgatacc

SEQ ID NO: 437 - RNA, 120 nt, synthetic construct
  source 1..120: mol_type = other RNA
gaaaccataggtagaggcgccaccaccttacatggtgccgataccgctccgttggtgcagtgtggactgtaatggtagaggctaaagagaggtgccaccggtttacatggcgccgatacc

SEQ ID NO: 438 - RNA, 122 nt, synthetic construct
  source 1..122: mol_type = other RNA
ttaataatctgattacggctgattgccgccggtagaggtgccaccgccttacatgacactgataccttatatccagccgtatttatggtagaggtgccaccggtttacatggcgccgatacc

SEQ ID NO: 439 - RNA, 121 nt, synthetic construct
  source 1..121: mol_type = other RNA
ttaataatctgattacggctgattgccgccggtagaggtgccaccgccttacatgacactgataccttatatccagccgtatttatggtagaggtgccaccgaaagcatggcgccgatacc

SEQ ID NO: 440 - RNA, 175 nt, synthetic construct
  source 1..175: mol_type = other RNA
aagaatactgatattctctatcggtccgaaaagtttgaattgttttggaatcgtaggccggtttgtgcgccaaccgcagaagagctggccttgctcacaatcaccagtgaaaacttgcggacagtatggaacgaatggaacggcctcctcaaagccaaccgagggggtaggctac

SEQ ID NO: 441 - RNA, 169 nt, synthetic construct
  source 1..169: mol_type = other RNA
aagaatactgatattctctatcggtccgaaaagtttgaattgttttggaatcgtaggccggtttgtgcgccaaccgcagaagagctggccttgctcacaatcaccagtgaaaacttgcggacagtatggaacgaatggaacggcctcctcaaaggagggggtaggctac

SEQ ID NO: 442 - RNA, 173 nt, synthetic construct
  source 1..173: mol_type = other RNA
tatgctcattgaaaacaaggcatgagaaaaatacccccggttttgggccggaatggaatgtttttctcactgccataccgtttttatgaggcaaccctttagggcactgcattgggcagtgtttatgcgctgttggaacggcctcctcaaagccaaccgagggggtaggctac

SEQ ID NO: 443 - RNA, 167 nt, synthetic construct
  source 1..167: mol_type = other RNA
tatgctcattgaaaacaaggcatgagaaaaatacccccggttttgggccggaatggaatgtttttctcactgccataccgtttttatgaggcaaccctttagggcactgcattgggcagtgtttatgcgctgttggaacggcctcctcaaaggagggggtaggctac

SEQ ID NO: 444 - RNA, 143 nt, synthetic construct
  source 1..143: mol_type = other RNA
cttatcgtgaggtgcagccacgatgtgcgaatctaaagtggacagaaatgggaacagacgttcgcacgaatgttggcggatttcttcggaaatcgagccaatcatcatatccatattatctgcggatggatgtgaactgcaag

SEQ ID NO: 445 - RNA, 150 nt, synthetic construct
  source 1..150: mol_type = other RNA
cttatcgtgaggtgcagccacgatgtgcgaatctaaagtgaacagaaatgggaacagacgttcgcacgaatgttggcggatttcttcggaaatcgagccaataatcatatccatattaccgggttgctgcggatggatgtgaactgcaag

SEQ ID NO: 446 - RNA, 131 nt, synthetic construct
  source 1..131: mol_type = other RNA
gtgaggtgcagccacgaggtgcgaataggaagtacgcagcaatgtgctgaatcgttcgcacgaaaattggcatttttgaaaaccaaagccaataatcatatccataaatatgtgtggttatgaactgcaag

SEQ ID NO: 447 - RNA, 186 nt, synthetic construct
  source 1..186: mol_type = other RNA
cttatcgtgaggtgcagccacgatgtgcgaatctaaagtgaacagaaatgggaacagacgttcgcacgaatgttggcggatttcttcggaaatcgagccaataatcatatccatattaccgggttgctctatagatagtgttgggtacacttgctgaacacactgcggatggatgtgaactgcaag

SEQ ID NO: 448 - RNA, 185 nt, synthetic construct
  source 1..185: mol_type = other RNA
cttatcgtgaggtgcagccacgatgtgcgaatctaaagtgaacagaaatgggaacagacgttcgcacgaatgttggcggatttcttcggaaatcgagccaataatcatatccatattaccgggttgctctatagatagtgtaaagacacttgctgaacacactgcggatggatgtgaactgcaag

SEQ ID NO: 449 - RNA, 195 nt, synthetic construct
  source 1..195: mol_type = other RNA
cttatcgtgaggtgcagccacgatgtgcgaatctaaagtgaacagaaatgggaacagacgttcgcacgaatgttggcggatttcttcggaaatcgagccaataatcatatccatattaccgggttgctctatagatagtgttgggtacacttgctgaacacaagatattgcctgcggatggatgtgaactgcaag

SEQ ID NO: 450 - RNA, 195 nt, synthetic construct
  source 1..195: mol_type = other RNA
cttatcgtgaggtgcagccacgatgtgcgaatctaaagtgaacagaaatgggaacagacgttcgcacgaatgttggcggatttcttcggaaatcgagccaataatcatatccatattaccgggttgctctatagatagtgttgggtaaaagtgctgaacacaagatattgcctgcggatggatgtgaactgcaag

SEQ ID NO: 451 - RNA, 154 nt, synthetic construct
  source 1..154: mol_type = other RNA
cttatcgtgaggtgcagccacgatgtgcgaatctaaagtgaacagaaatgggaacagacgttcgcacgaatgttggcggatttcttcggaaatcgagccaataatcatatccatattaccgggttgctctctgcggatggatgtgaactgcaag

SEQ ID NO: 452 - RNA, 150 nt, synthetic construct
  source 1..150: mol_type = other RNA
cttatcgtgaggtgcagccacgatgtgcgaatctaaagtgaacagaaatgggaacagacgttcgcacgaatgttggcggatttcttcggaaatcgagccaataatcatatccatattaccgggaaagtgcggatggatgtgaactgcaag

SEQ ID NO: 453 - RNA, 290 nt, synthetic construct
  source 1..290: mol_type = other RNA
taccgcagtttgggcgggttgctaataatattagcttatgacttctaaggcttttgcctaaaaagtaagggggaaggcgacaacccccaaagaccagccggaactatggctggcaactatctcatctttttggcatatcaaagctagggcaaaaaccccagacattcgccaaaagcccagaaccatgacattgcaagagtttcgcccagtttcttttaaagacccaagctgatttaagcggctgaaatgagatcttgcaactgggcttggggactgaggatagttgaaac

SEQ ID NO: 454 - RNA, 291 nt, synthetic construct
  source 1..291: mol_type = other RNA
taccgcagtttgggcgggttgctaataatattagcttatgacttctaaggcttttgcctaaaaagtaagggggaaggcgacaacccccaaagaccagccggaactatggctggcaactatctcatctttttggcatatcaaagctagggcaaaaaccccagacattcgccaaaagcccagaaccatgacattgcaagagtttcgcccagtttcttaaagaagacccaagctgatttaagcggctgaaatgagatcttgcaactgggcttggggactgaggatagttgaaac

SEQ ID NO: 455 - RNA, 320 nt, synthetic construct
  source 1..320: mol_type = other RNA
taccgcagtttgggcgggttgctaataatattagcttatgacttctaaggcttttgcctaaaaagtaagggggaaggcgacaacccccaaagaccagccggaactatggctggcaactatctcatctttttggcatatcaaagctagggcaaaaaccccagacattcgccaaaagcccagaaccatgacattgcaagagtttcgcccagtttcttttaaagacccaagctgatttaagcggctgaaatgagattttttaacaggtccgccaaaatcgcttctgcttgcaactgggcttggggactgaggatagttgaaac

SEQ ID NO: 456 - RNA, 320 nt, synthetic construct
  source 1..320: mol_type = other RNA
taccgcagtttgggcgggttgctaataatattagcttatgacttctaaggcttttgcctaaaaagtaagggggaaggcgacaacccccaaagaccagccggaactatggctggcaactatctcatctttttggcatatcaaagctagggcaaaaaccccagacattcgccaaaagcccagaaccatgacattgcaagagtttcgcccagtttcttttaaagacccaagctgatttaagcggctgaaatgagaaagtttaacaggtccgccaaaatcgcttctgcttgcaactgggcttggggactgaggatagttgaaac

SEQ ID NO: 457 - RNA, 137 nt, synthetic construct
  source 1..137: mol_type = other RNA
ataagatggactatatttataaaccaggaaacatagttattacaattgcattgtaggtttcaacctgatacccactaaagcgtgttagtgggtattttttcttgcaactgggcttggggactgaggatagttgaaac

SEQ ID NO: 458 - RNA, 135 nt, synthetic construct
  source 1..135: mol_type = other RNA
ataagatggactatatttataaaccaggaaacatagttattacaattgcattgtaggtttcaacctgatacccactaaaaagttagtgggtattttttcttgcaactgggcttggggactgaggatagttgaaac

SEQ ID NO: 459 - RNA, 157 nt, synthetic construct
  source 1..157: mol_type = other RNA
cttgacagtaaacaaaaaataagatggactatatttataaaccaggaaacatagttattacaattgcattgtaggtttcaacctgatacccactaaagcgtgttagtgggtattttttatcttgcaactgggcttggggactgaggatagttgaaac

SEQ ID NO: 460 - RNA, 158 nt, synthetic construct
  source 1..158: mol_type = other RNA
cttgacagtaaacaaaaaataagatggactatatttataaaccaggaaacatagttattacaattgcattgtaggtttcaacctgatacccactaaagcgtgttagtgggtattttttatcttgcaactgggcttggaaagctgaggatagttgaaac

SEQ ID NO: 461 - RNA, 174 nt, synthetic construct
  source 1..174: mol_type = other RNA
agccgcacggaacctgagccgatggcgtagcccttggacctatatggaacgcggcataagccctgcgagttcgcaagagcccaaggcggcatgacaagcctctttcaggcgacagagtcttttggagtgtcgaggctccctgcattccttgggagcctcccaaaggaatgaaag

SEQ ID NO: 462 - RNA, 168 nt, synthetic construct
  source 1..168: mol_type = other RNA
agccgcacggaacctgagccgatggcgtagcccttggacctatatggaacgcggcataagccctgcgagttcgcaagagcccaaggcggcatgacaagcctctttcaggcgacagagtcttttggagtgtcgaggctcccaaaggggagcctcccaaaggaatgaaag

SEQ ID NO: 463 - RNA, 106 nt, synthetic construct
  source 1..106: mol_type = other RNA
agccgcacggaacctgagccgatggcgtagcccttggacctatatggaacgcggcataagccctgcgagttcgcaagagcccaaggcggcatgaaaggaatgaaag

SEQ ID NO: 464 - RNA, 106 nt, synthetic construct
  source 1..106: mol_type = other RNA
agccgcacggaacctgagccgatggcgtagcccttggacctatatggaacgcggcataagccctgcgagttcaaaggagcccaaggcggcatgaaaggaatgaaag

SEQ ID NO: 465 - RNA, 163 nt, synthetic construct
  source 1..163: mol_type = other RNA
gttccgtctcgactatgccgtaccactagaccgagcctacacggcacgcggtcatagcgttaaccaaggcgtggtgacaagcctctttcaggcgtcggacacttaagagcgttaggcgggcggtccctaagccgcccgcccccacgctcttagggaatgaaag

SEQ ID NO: 466 - RNA, 161 nt, synthetic construct
  source 1..161: mol_type = other RNA
gttccgtctcgactatgccgtaccactagaccgagcctacacggcacgcggtcatagcgttaaccaaggcgtggtgacaagcctctttcaggcgtcggacacttaagagcgttaggcgggcggtaaaggccgcccgcccccacgctcttagggaatgaaag

SEQ ID NO: 467 - RNA, 193 nt, synthetic construct
  source 1..193: mol_type = other RNA
gttccgtctcgactatgccgtaccactagaccgagcctacacggcacgcggtcatagcgttaaccaaggcgtggtgacaagcctctttcaggcgtcggacacttaagagcgttaggcgggcggtccctaagccgcccgcccccttatttgcacgttttccccgaaccccgtaaacgctcttagggaatgaaag

SEQ ID NO: 468 - RNA, 192 nt, synthetic construct
  source 1..192: mol_type = other RNA
gttccgtctcgactatgccgtaccactagaccgagcctacacggcacgcggtcatagcgttaaccaaggcgtggtgacaagcctctttcaggcgtcggacacttaagagcgttaggcgggcggtccctaagccgcccgcccccttatttgcacgtttaaaggaaccccgtaaacgctcttagggaatgaaag

SEQ ID NO: 469 - RNA, 268 nt, synthetic construct
  source 1..268: mol_type = other RNA
gttccgtctcgactatgccgtaccactagaccgagcctacacggcacgcggtcatagcgttaaccaaggcgtggtgacaagcctctttcaggcgtcggacacttaagagcgttaacgctcttagggaatgaaaggttccgtctcgactatgccgtaccactagaccgagcctacacggcacgcggtcatagcgttaaccaaggcgtggtgacaagcctctttcaggcgtcggacacttaagagcgaaagcgctcttagggaatgaaag

SEQ ID NO: 470 - DNA, 20 bp, synthetic construct
  source 1..20: mol_type = other DNA
cagctgacttggatgctgga

SEQ ID NO: 471 - DNA, 20 bp, synthetic construct
  source 1..20: mol_type = other DNA
cttctctctgtccctccctt

SEQ ID NO: 472 - DNA, 20 bp, synthetic construct
  source 1..20: mol_type = other DNA
cacccagttgtcatggcgat

SEQ ID NO: 473 - DNA, 20 bp, synthetic construct
  source 1..20: mol_type = other DNA
ccaggtgggcaaacacgatt

SEQ ID NO: 474 - DNA, 20 bp, synthetic construct
  source 1..20: mol_type = other DNA
ttgagggaaaaatccaactg

SEQ ID NO: 475 - DNA, 20 bp, synthetic construct
  source 1..20: mol_type = other DNA
tgttgccctcataacttatc

SEQ ID NO: 476 - DNA, 20 bp, synthetic construct
  source 1..20: mol_type = other DNA
tcctgctgacatgacaaata

SEQ ID NO: 477 - DNA, 20 bp, synthetic construct
  source 1..20: mol_type = other DNA
tgtcgcagttgcaaatgaag

SEQ ID NO: 478 - DNA, 20 bp, synthetic construct
  source 1..20: mol_type = other DNA
tagaagctgatctgaaggga

SEQ ID NO: 479 - RNA, 163 nt, synthetic construct
  source 1..163: mol_type = other RNA
cttatcgtgaggtgcagccacgatgtgcgaatctaaagtggacagaaatgggaacagacgttcgcacgaatgttggcggatttcttcggaaatcgagccaatcatcatatccatattatctgcggatggatgtgaactgcaagcagctgacttggatgctgga

SEQ ID NO: 480 - RNA, 170 nt, synthetic construct
  source 1..170: mol_type = other RNA
cttatcgtgaggtgcagccacgatgtgcgaatctaaagtgaacagaaatgggaacagacgttcgcacgaatgttggcggatttcttcggaaatcgagccaataatcatatccatattaccgggttgctgcggatggatgtgaactgcaagcagctgacttggatgctgga

SEQ ID NO: 481 - RNA, 163 nt, synthetic construct
  source 1..163: mol_type = other RNA
cttatcgtgaggtgcagccacgatgtgcgaatctaaagtggacagaaatgggaacagacgttcgcacgaatgttggcggatttcttcggaaatcgagccaatcatcatatccatattatctgcggatggatgtgaactgcaagcttctctctgtccctccctt

SEQ ID NO: 482 - RNA, 170 nt, synthetic construct
  source 1..170: mol_type = other RNA
cttatcgtgaggtgcagccacgatgtgcgaatctaaagtgaacagaaatgggaacagacgttcgcacgaatgttggcggatttcttcggaaatcgagccaataatcatatccatattaccgggttgctgcggatggatgtgaactgcaagcttctctctgtccctccctt

SEQ ID NO: 483 - RNA, 163 nt, synthetic construct
  source 1..163: mol_type = other RNA
cttatcgtgaggtgcagccacgatgtgcgaatctaaagtggacagaaatgggaacagacgttcgcacgaatgttggcggatttcttcggaaatcgagccaatcatcatatccatattatctgcggatggatgtgaactgcaagcacccagttgtcatggcgat

SEQ ID NO: 484 - RNA, 170 nt, synthetic construct
  source 1..170: mol_type = other RNA
cttatcgtgaggtgcagccacgatgtgcgaatctaaagtgaacagaaatgggaacagacgttcgcacgaatgttggcggatttcttcggaaatcgagccaataatcatatccatattaccgggttgctgcggatggatgtgaactgcaagcacccagttgtcatggcgat

SEQ ID NO: 485 - RNA, 163 nt, synthetic construct
  source 1..163: mol_type = other RNA
cttatcgtgaggtgcagccacgatgtgcgaatctaaagtggacagaaatgggaacagacgttcgcacgaatgttggcggatttcttcggaaatcgagccaatcatcatatccatattatctgcggatggatgtgaactgcaagccaggtgggcaaacacgatt

SEQ ID NO: 486 - RNA, 170 nt, synthetic construct
  source 1..170: mol_type = other RNA
cttatcgtgaggtgcagccacgatgtgcgaatctaaagtgaacagaaatgggaacagacgttcgcacgaatgttggcggatttcttcggaaatcgagccaataatcatatccatattaccgggttgctgcggatggatgtgaactgcaagccaggtgggcaaacacgatt

SEQ ID NO: 487 - RNA, 163 nt, synthetic construct
  source 1..163: mol_type = other RNA
cttatcgtgaggtgcagccacgatgtgcgaatctaaagtggacagaaatgggaacagacgttcgcacgaatgttggcggatttcttcggaaatcgagccaatcatcatatccatattatctgcggatggatgtgaactgcaagttgagggaaaaatccaactg

SEQ ID NO: 488 - RNA, 170 nt, synthetic construct
  source 1..170: mol_type = other RNA
cttatcgtgaggtgcagccacgatgtgcgaatctaaagtgaacagaaatgggaacagacgttcgcacgaatgttggcggatttcttcggaaatcgagccaataatcatatccatattaccgggttgctgcggatggatgtgaactgcaagttgagggaaaaatccaactg

SEQ ID NO: 489 - RNA, 163 nt, synthetic construct
  source 1..163: mol_type = other RNA
cttatcgtgaggtgcagccacgatgtgcgaatctaaagtggacagaaatgggaacagacgttcgcacgaatgttggcggatttcttcggaaatcgagccaatcatcatatccatattatctgcggatggatgtgaactgcaagtgttgccctcataacttatc

SEQ ID NO: 490 - RNA, 170 nt, synthetic construct
  source 1..170: mol_type = other RNA
cttatcgtgaggtgcagccacgatgtgcgaatctaaagtgaacagaaatgggaacagacgttcgcacgaatgttggcggatttcttcggaaatcgagccaataatcatatccatattaccgggttgctgcggatggatgtgaactgcaagtgttgccctcataacttatc

SEQ ID NO: 491 - RNA, 163 nt, synthetic construct
  source 1..163: mol_type = other RNA
cttatcgtgaggtgcagccacgatgtgcgaatctaaagtggacagaaatgggaacagacgttcgcacgaatgttggcggatttcttcggaaatcgagccaatcatcatatccatattatctgcggatggatgtgaactgcaagtcctgctgacatgacaaata

SEQ ID NO: 492 - RNA, 170 nt, synthetic construct
  source 1..170: mol_type = other RNA
cttatcgtgaggtgcagccacgatgtgcgaatctaaagtgaacagaaatgggaacagacgttcgcacgaatgttggcggatttcttcggaaatcgagccaataatcatatccatattaccgggttgctgcggatggatgtgaactgcaagtcctgctgacatgacaaata

SEQ ID NO: 493 - RNA, 163 nt, synthetic construct
  source 1..163: mol_type = other RNA
cttatcgtgaggtgcagccacgatgtgcgaatctaaagtggacagaaatgggaacagacgttcgcacgaatgttggcggatttcttcggaaatcgagccaatcatcatatccatattatctgcggatggatgtgaactgcaagtgtcgcagttgcaaatgaag

SEQ ID NO: 494 - RNA, 170 nt, synthetic construct
  source 1..170: mol_type = other RNA
cttatcgtgaggtgcagccacgatgtgcgaatctaaagtgaacagaaatgggaacagacgttcgcacgaatgttggcggatttcttcggaaatcgagccaataatcatatccatattaccgggttgctgcggatggatgtgaactgcaagtgtcgcagttgcaaatgaag

SEQ ID NO: 495 - RNA, 163 nt, synthetic construct
  source 1..163: mol_type = other RNA
cttatcgtgaggtgcagccacgatgtgcgaatctaaagtggacagaaatgggaacagacgttcgcacgaatgttggcggatttcttcggaaatcgagccaatcatcatatccatattatctgcggatggatgtgaactgcaagtagaagctgatctgaaggga

SEQ ID NO: 496 - RNA, 170 nt, synthetic construct
  source 1..170: mol_type = other RNA
cttatcgtgaggtgcagccacgatgtgcgaatctaaagtgaacagaaatgggaacagacgttcgcacgaatgttggcggatttcttcggaaatcgagccaataatcatatccatattaccgggttgctgcggatggatgtgaactgcaagtagaagctgatctgaaggga